(19) United States
(12) Patent Application Publication
(10) Pub. No.: US 2008/0045521 A1
(43) Pub. Date: Feb. 21, 2008

(54) PHENYLALANINE DERIVATIVES

(75) Inventors: Jean-Claude Arnould, Reims (FR); Benedicte Delouvrie, Reims (FR); Richard Ducray, Reims (FR); Christine Marie Paul Lambert Van Der Brempt, Reims (FR)

Correspondence Address: ASTRAZENECA R&D BOSTON, 35 GATEHOUSE DRIVE, WALTHAM, MA, 02451-1215, US

(73) Assignee: AstraZeneca AB, Sodertalje (SE)

(21) Appl. No.: 11/746,892

(22) Filed: May 10, 2007

(30) Foreign Application Priority Data
Jun. 9, 2006 (EP) 06300576.3
Dec. 12, 2006 (EP) 06301245.4
Apr. 23, 2007 (EP) 07300973.0

(51) Int. Cl. A61K 31/44; A61K 31/4375; A61K 31/4436; A61K 31/444; A61K 31/5383; A61P 35/00; C07D 213/72; C07D 401/06; C07D 409/06; C07D 487/04; C07D 498/02

(52) U.S. Cl. 514/230.5; 514/300; 514/332; 514/336; 514/352; 544/105; 546/122; 546/264; 546/280.4; 546/312

(57) ABSTRACT

The present invention relates to compounds that inhibit of a5b1 function, processes for their preparation, pharmaceutical compositions containing them as the active ingredient, to their use as medicaments and to their use in the manufacture of medicaments for use in the treatment in warm-blooded animals such as humans of diseases that have a significant angiogenesis or vascular component such as for treatment of solid tumours. The present invention also relates to a5b1 antagonists that also exhibit appropriate selectivity profile(s) against other integrins.

### RELATED APPLICATIONS

This application claims the benefit under 35 USC §119(a)-(d) of European Patent Application No. 06300576.3, filed on Jun. 9, 2006; European Patent Application No. 06301245.4, filed on Dec. 12, 2006; and European Patent Application No. 07300973.0, filed on Apr. 23, 2007.

### FIELD OF INVENTION

The present invention relates to chemical compounds useful as pharmaceuticals in particular in the treatment of diseases in which α5β1 function is a factor, to process for their preparation, and to compositions containing these as well as their use in therapy.

### BACKGROUND OF THE INVENTION

Many normal physiological and disease processes require cells to contact other cells and/or extracellular matrix. Cell-matrix and cell-cell adhesion is mediated through several families of proteins including integrins, selectins, cadherins, and immunoglobulins, and facilitates a variety of normal cellular functions such as proliferation, migration, differentiation or survival. Cell adhesion is also key to a range of pathologies, and so pharmacological disruption of cell adhesion interactions can provide a mechanism for therapeutic intervention. In particular members of the integrin superfamily of adhesion molecules are believed to play a particularly important role in acute and chronic disease states such as cancer, inflammatory diseases, stroke and neurodegenerative disorders^{(1,2)}. Thus, integrins represent a very complex biological area.

The integrin superfamily of cell surface receptors is formed from a number of structurally and functionally related surface glycoproteins, with each receptor existing as a heterodimer of non-covalently linked α and β subunits. To date, at least 18 different α and 8β subunits have been identified in mammals, which are known to form more than 24 different receptors. Each integrin interacts specifically with defined extracellular ligands, including extracellular matrix proteins such as, fibronectin, fibrinogen, vitronectin, collagen and cell surface molecules such as VCAM, ICAM and PECAM, via linear adhesion motifs.

The integrin α5β1 (hereinafter α5β1) is composed of an α5 (hereinafter α5) and β1 (hereinafter b1) subunits, the a5 subunit forming a specific dimer with the b1 subunit, and is widely expressed in most tissues⁽³⁾. Integrin a5b1 almost exclusively mediates cell adhesion through an interaction with fibronectin, binding via the short arginine-glycine-aspartate (RGD) adhesion motif. Endothelial cells can however bind to fibrin via α5β1. There is compelling evidence that the a5b 1 interaction with fibronectin plays an important role in physiopathological angiogenesis and vascular integrity^{(4,5)}. Although endothelial cells express a variety of integrins, a5b 1 is important for survival of endothelial cells on provisional matrix in vitro, suppressing apoptosis and promoting proliferation. Furthermore, immunohistochemical analysis, and imaging have both shown that a5b 1 expression is upregulated in tumour vasculature^{(4,6)}. Consistent with a key functional role for the receptor-ligand pairing, the a5b1 ligand fibronectin is also upregulated in tumour tissue and during wound-healing⁽⁴⁾. Transgenic studies further support an important role for a5b1 in the vasculature. Both a5 and b1 knock-out mice are embryonic lethal and display defects in development of early vascular systems, suggesting a pivotal functional role in early vasculogenesis^{(7,8)}. Moreover, studies using agents such as blocking RGD is peptides or neutralising antibodies have shown that disruption of a5b1 interaction with its cognate ligands has anti-angiogenic effects in vivo⁽⁴⁾. As well as inhibiting angiogenesis, a5b1 inhibitors may reduce the proliferation of certain tumour cells that express the receptor.

In addition to a5b1, other integrin family members such as avb3 and aiibb3 can also interact with RGD-containing ligands⁽¹⁾. Other integrins can bind to ligands via non-RGD binding domains. An example of particular importance and relevance is a4b1 which binds via a leucine-aspartate-valine (LDV) motif to ligands that include the connecting segment-1 region of fibronectin, VCAM-1, MAdCAM or to the SVVYGLR motif found within osteopontin.

Since there are a variety of integrins that share the same ligand or binding-domain with a5b1, it will be important to develop therapeutic agents that are selective towards a5b1 activity. However, as other endothelial integrins such as avb3, avb5 and a4b1 are also involved in possible pathological events, agents which target such integrins in addition to a5b1, may have additional therapeutic activity.

### REFERENCES

1. Shimaoka, M et al. (2003) Nat. Rev. Drug. Disc. 2, 703-716
2. Mousa, S A (2002) Curr. Opin. Chem. Biol. 6:534-541
3. Parsons-Wingerter, P et al. (2005) Am. J. Path. 167(1), 193-211
4. Kim, S et al. (2000) Am. J. Path. 156(4), 1345-1362
5. Ramakrishnan, V et al. (2006) J. Exp. Ther. One. 5, 273-286
6. Magnussen, A et al. (2005) Cancer Res. 65(7), 2712-2721
7. Goh, K L et al. (1997) Development 124, 4309-4319
8. Yang, J T et al. (1993) Development 119, 1093-1105

Taken together, the expression and functional data suggest that selective inhibition of a5b1 function provides an attractive therapeutic strategy to combat diseases that have a significant angiogenesis or vascular component such as for treatment of solid tumours or other pathological angiogenic conditions such as age-related macular degeneration.

A number of small-molecule a5b1 antagonists are known, for example WO97/33887 describe spirocyclic compounds, and WO2005/090329 describes substituted pyrrolidines and other cyclic and heterocyclic compounds. There are a number of a5b1 antagonists in development, for example JSM6427 and SJ749. There remains however the need to develop alternative a5b1 antagonists.

There is thus a clear need to develop compounds that are a5b1 antagonists with appropriate pharmacokinetic and pharmacodynamic drug properties, and also that exhibit appropriate selectivity profile(s) against other integrins.

### SUMMARY OF THE INVENTION

These and other needs are met by the present invention which is directed to a compound of formula I: 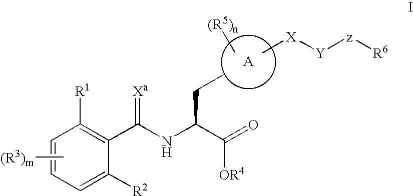
wherein:

X^{a} is selected from oxygen or sulphur;

R¹ is selected from bromo, chloro, (1-3C)alkyl, cyclopropyl, cyclobutyl, cyclopentyl, cyclopropylmethyl, cyclobutylmethyl, cyclopentylmethyl and halo-(1-3C)alkyl;

R² and each R³, which may be the same or different, is selected from hydrogen, halo, trifluoromethyl, cyano, isocyano, nitro, hydroxy, mercapto, amino, formyl, carboxy, carbamoyl, sulfamoyl, (1-6C)alkyl, (2-8C)alkenyl, (2-8C)alkynyl, (1-6C)alkoxy, (2-6C)alkenyloxy, (2-6C)alkynyloxy, (1-6C)alkylthio, (1-6C)alkylsulfinyl, (1-6C)alkylsulfonyl, (1-6C)alkylamino, di-[(1-6C)alkyl]amino, (1-6C)alkoxycarbonyl, N-(1-6C)alkylcarbamoyl, N,N-di-[(1-6C)alkyl]carbamoyl, (2-6C)alkanoyl, (2-6C)alkanoyloxy, (2-6C)alkanoylamino, N-(1-6C)alkyl-(2-6C)alkanoylamino, (3-6C)alkenoylamino, N-(1-6C)alkyl-(3-6C)alkenoylamino, (3-6C)alkynoylamino, N-(1-6C)alkyl-(3-6C)alkynoylamino, N-(1-6C)alkylsulfamoyl, N,N-di-[(1-6C)alkyl]sulfamoyl, (1-6C)alkanesulfonylamino and N-(1-6C)alkyl-(1-6C)alkanesulfonylamino,

or from a group of the formula:
-Q¹-X¹
wherein X¹ is a direct bond or is selected from O, S, SO, SO₂, N(R⁷), C(O), CH(OR⁷), C(O)N(R⁷), N(R⁷)C(O), SO₂N(R⁷), N(R⁷)SO₂, OC(R⁷)₂, SC(R⁷)₂ and N(R⁷)C(R⁷)₂, wherein R⁷ is hydrogen or (1-6C)alkyl, and Q¹ is aryl, aryl-(1-6C)alkyl, (3-7C)cycloalkyl, (3-7C)cycloalkyl-(1-6C)alkyl, (3-7C)cycloalkenyl, (3-7C)cycloalkenyl-(1-6C)alkyl, heteroaryl, heteroaryl-(1-6C)alkyl, heterocyclyl or heterocyclyl-(1-6C)alkyl,

and wherein R² and any R³ independently of each other optionally bears on carbon one or more R⁸ groups,

and wherein if any heteroaryl or heterocyclyl group within R² and any R³ contains an —NH— moiety, the nitrogen of said moiety optionally bears a group selected from R⁹,

and wherein any heterocyclyl group within R² and any R³ optionally bears 1 or 2 oxo or thioxo substituents;

or R² and an R³ substituent optionally form a (1-3C)alkylenedioxy group;

or two R³ substituents optionally form a (1-3C)alkylenedioxy group;

m is 0, 1, 2 or 3;

R⁴ is selected from hydrogen, (1-6C)alkyl, heterocyclyl, heterocyclyl-(1-6C)alkyl, aryl, aryl-(1-6C)alkyl, heteroaryl and heteroaryl-(1-6C)alkyl, which optionally bears on carbon one or more R²¹ substituents, which may be the same or different,

and wherein if any heteroaryl or heterocyclyl group within R⁴ contains an —NH-moiety, the nitrogen of said moiety optionally bears a group selected from R²²,

and wherein and wherein any heterocyclyl group within R⁴ optionally bears 1 or 2 oxo or thioxo substituents; 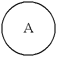
is selected from phenyl, pyridinyl and thiophenyl;

n is 0, 1, 2, 3 or 4, provided that when ring A is pyridinyl, n is 0, 1, 2 or 3 and that when ring A is thiophenyl, n is 0, 1 or 2;

each R⁵, which may be the same or different, is selected from halo, trifluoromethyl, cyano, isocyano, nitro, hydroxy, mercapto, amino, formyl, carboxy, carbamoyl, sulfamoyl, (1-6C)alkyl, (2-8C)alkenyl, (2-8C)alkynyl, (1-6C)alkoxy, (2-6C)alkenyloxy, (2-6C)alkynyloxy, (1-6C)alkylthio, (1-6C)alkylsulfinyl, (1-6C)alkylsulfonyl, (1-6C)alkylamino, di-[(1-6C)alkyl]amino, (1-6C)alkoxycarbonyl, N-(1-6C)alkylcarbamoyl, N,N-di-[(1-6C)alkyl]carbamoyl, (2-6C)alkanoyl, (2-6C)alkanoyloxy, (2-6C)alkanoylamino, N-(1-6C)alkyl-(2-6C)alkanoylamino, (3-6C)alkenoylamino, N-(1-6C)alkyl-(3-6C)alkenoylamino, (3-6C)alkynoylamino, N-(1-6C)alkyl-(3-6C)alkynoylamino, N-(1-6C)alkylsulfamoyl, N,N-di-[(1-6C)alkyl]sulfamoyl, (1-6C)alkanesulfonylamino and N-(1-6C)alkyl-(1-6C)alkanesulfonylamino,

or from a group of the formula:
Q⁵-X⁷—
wherein X⁷ is a direct bond or is selected from O, S, SO, SO₂, N(R²³), C(O), CH(OR²³), C(O)N(R²³), N(R²³)C(O), SO₂N(R²³), N(R²³)SO₂, OC(R²³)₂, SC(R²³)₂ and N(R²³)C(R²³)₂, wherein R²³ is hydrogen or (1-6C)alkyl, and Q⁵ is aryl, aryl-(1-6C)alkyl, (3-7C)cycloalkyl, (3-7C)cycloalkyl-(1-6C)alkyl, (3-7C)cycloalkenyl, (3-7C)cycloalkenyl-(1-6C)alkyl, heteroaryl, heteroaryl-(1-6C)alkyl, heterocyclyl or heterocyclyl-(1-6C)alkyl,

and wherein R⁵ optionally bears on carbon one or more R²⁴ groups,

and wherein if any heteroaryl or heterocyclyl group within R⁵ contains an —NH-moiety, the nitrogen of said moiety optionally bears a group selected from R²⁵,

and wherein any heterocyclyl group within R⁵ optionally bears 1 or 2 oxo or thioxo substituents;

or two R⁵ substituents optionally form a (1-3C)alkylenedioxy group;

X is selected from a direct bond, N(R²⁶), O, S, SO, SO₂, C(O), CH(OR²⁶), C(O)N(R²⁶), N(R²⁶)C(O), SO₂N(R²⁶), N(R²⁶)SO₂, (1-6C)alkylene, CH═CH and C≡C, wherein R²⁶ is hydrogen, (1-6C)alkyl or (3-7C)cycloalkyl;

Y is selected from (1-6C)alkylene, (3-7C)cycloalkylene, (3-7C)cycloalkenylene and heterocyclyl,

Z is selected from a direct bond, N(R²⁶), O, S, SO, SO₂, C(O), CH(OR²⁶), SO₂N(R²⁶), N(R²⁶)SO₂, (1-6C)alkylene, CH═CH and C≡C, wherein R²⁶ is hydrogen, (1-6C)alkyl or (3-7C)cycloalkyl;

and wherein adjacent carbon atoms in any (2-6C)alkylene chain within an X, Y or Z substituent are optionally separated by the insertion into the chain of a group selected from O, S, SO, SO₂, N(R²⁷), C(O), CH(OR²⁷), C(O)N(R²⁷), N(R²⁷)C(O), SO₂N(R²⁷), N(R²⁷)SO₂, CH═CH and C≡C wherein R²⁷ is hydrogen, (1-6C)alkyl or (3-7C)cycloalkyl;

and wherein any X, Y or Z optionally bears on carbon one or more R²⁸ substituents,

and wherein if any heterocyclyl group within Y contains an —NH— moiety, the nitrogen of said moiety optionally bears a group selected from R²⁹;

provided that when X is a direct bond or O and Y is a heterocyclyl containing a nitrogen heteroatom, then the group R⁶-Z- is attached to a carbon atom in the heterocyclyl containing the nitrogen heteroatom;

R⁶ is heteroaryl, which heteroaryl contains at least one nitrogen atom, and wherein R⁶ optionally bears on carbon one or more R³¹ substituents,

and wherein if R⁶ contains an —NH— moiety, the nitrogen of said moiety optionally bears a group selected from R³⁵;

R⁸, R²¹, R²⁴ and R²⁸ is selected from halo, trifluoromethyl, cyano, nitro, hydroxy, amino, carboxy, carbamoyl, sulfamoyl, (1-6C)alkyl, (2-8C)alkenyl, (2-8C)alkynyl, (1-6C)alkoxy, (2-6C)alkenyloxy, (2-6C)alkynyloxy, (1-6C)alkylthio, (1-6C)alkylsulfinyl, (1-6C)alkylsulfonyl, (1-6C)alkylamino, di-[(1-6C)alkyl]amino, (1-6C)alkoxycarbonyl, N-(1-6C)alkylcarbamoyl, N,N-di-[(1-6C)alkyl]carbamoyl, (2-6C)alkanoyl, (2-6C)alkanoyloxy, (2-6C)alkanoylamino, N-(1-6C)alkyl-(2-6C)alkanoylamino, N-(1-6C)alkylsulfamoyl, N,N-di-[(1-6C)alkyl]sulfamoyl, (1-6C)alkanesulfonylamino and N-(1-6C)alkyl-(1-6C)alkanesulfonylamino,

or from a group of the formula:
—X²—R¹⁰
wherein X² is a direct bond or is selected from O, C(O) and N(R¹¹), wherein R¹¹ is hydrogen or (1-6C)alkyl, and R¹⁰ is halo-(1-6C)alkyl, hydroxy-(1-6C)alkyl, (1-6C)alkoxy-(1-6C)alkyl, cyano-(1-6C)alkyl, amino-(1-6C)alkyl, (1-6C)alkylamino-(1-6C)alkyl, di-[(1-6C)alkyl]amino-(1-6C)alkyl, (2-6C)alkanoylamino-(1-6C)alkyl and (1-6C)alkoxycarbonylamino-(1-6C)alkyl,

or from a group of the formula:
—X³-Q²
wherein X³ is a direct bond or is selected from O, S, SO, SO₂, N(R¹²), C(O), CH(OR¹²), C(O)N(R¹²), N(R¹²)C(O), SO₂N(R¹²), N(R¹²)SO₂, OC(R¹²)₂, SC(R¹²)₂ and N(R¹²)C(R¹²)₂, wherein R¹² is hydrogen or (1-6C)alkyl, and Q² is aryl, aryl-(1-6C)alkyl, (3-7C)cycloalkyl, (3-7C)cycloalkyl-(1-6C)alkyl, (3-7C)cycloalkenyl, (3-7C)cycloalkenyl-(1-6C)alkyl, heteroaryl, heteroaryl-(1-6C)alkyl, heterocyclyl or heterocyclyl-(1-6C)alkyl,

and wherein R⁸, R²¹, R²⁴ and R²⁸ independently of each other optionally bears on carbon one or more R¹³,

and wherein if any heteroaryl or heterocyclyl group within R⁸, R²¹, R²⁴ and R²⁸ contains an —NH— moiety, the nitrogen of said moiety optionally bears a group selected from R¹⁴,

and wherein any heterocyclyl group within a substituent on R⁸, R²¹, R²⁴ and R²⁸ independently of each other optionally bears 1 or 2 oxo or thioxo substituents;

R⁹, R²², R²⁵ and R²⁹ are each independently selected from cyano, hydroxy, carboxy, carbamoyl, sulfamoyl, (1-6C)alkyl, (2-8C)alkenyl, (2-8C)alkynyl, (1-6C)alkylsulfonyl, (1-6C)alkoxycarbonyl, N-(1-6C)alkylcarbamoyl, N,N-di-[(1-6C)alkyl]carbamoyl, (2-6C)alkanoyl, N-(1-6C)alkylsulfamoyl and N,N-di-[(1-6C)alkyl]sulfamoyl,

or from a group of the formula:
—X⁴—R¹⁵
wherein X⁴ is a direct bond or is selected from C(O), SO₂, C(O)N(R¹⁶) and SO₂N(R¹⁶), wherein R¹⁶ is hydrogen or (1-6C)alkyl, and R¹⁵ is halo-(1-6C)alkyl, hydroxy-(1-6C)alkyl, (1-6C)alkoxy-(1-6C)alkyl, cyano-(1-6C)alkyl, amino-(1-6C)alkyl, (1-6C)alkylamino-(1-6C)alkyl, di-[(1-6C)alkyl]amino-(1-6C)alkyl, (2-6C)alkanoylamino-(1-6C)alkyl and (1-6C)alkoxycarbonylamino-(1-6C)alkyl,

or from a group of the formula:
—X⁵-Q³
wherein X⁵ is a direct bond or is selected from C(O), SO₂, C(O)N(R¹⁷) and SO₂N(R¹⁷), wherein R¹⁷ is hydrogen or (1-6C)alkyl, and Q³ is aryl, aryl-(1-6C)alkyl, (3-7C)cycloalkyl, (3-7C)cycloalkyl-(1-6C)alkyl, (3-7C)cycloalkenyl, (3-7C)cycloalkenyl-(1-6C)alkyl, heteroaryl, heteroaryl-(1-6C)alkyl, heterocyclyl or heterocyclyl-(1-6C)alkyl,

and wherein R⁹, R²², R²⁵ and R²⁹ independently of each other optionally bears on carbon one or more R¹⁸,

and wherein if any heteroaryl or heterocyclyl group within R⁹, R²², R²⁵ and R²⁹ contains an —NH— moiety, the nitrogen of said moiety optionally bears a group selected from R¹⁹,

and wherein any heterocyclyl group within a substituent on R⁹, R²², R²⁵ and R²⁹ optionally bears 1 or 2 oxo or thioxo substituents;

R¹³ and R¹⁸ are each independently selected from halo, cyano, hydroxy, carboxy, amino, (3-6C)cycloalkyl, (2-6C)alkenyl, (2-6C)alkynyl, (1-6C)alkoxy, (1-6C)alkylamino and di-[(1-6C)alkyl]amino;

R¹⁴ and R¹⁹ are each independently selected from carbamoyl, sulfamoyl, (1-6C)alkyl, (2-8C)alkenyl, (2-8C)alkynyl, (1-6C)alkylsulfonyl, N-(1-6C)alkylcarbamoyl, N,N-di-[(1-6C)alkyl]carbamoyl, (2-6C)alkanoyl, N-(1-6C)alkylsulfamoyl and N,N-di-[(1-6C)alkyl]sulfamoyl,

or from a group of the formula:
—X⁶-Q⁴

wherein X⁶ is a direct bond or is selected from C(O), SO₂, C(O)N(R²⁰) and SO₂N(R²⁰), wherein R²⁰ is hydrogen or (1-6C)alkyl, and Q⁴ is (3-7C)cycloalkyl or (3-7C)cycloalkyl-(1-6C)alkyl;

R³¹ is selected from halo, cyano, hydroxy, nitro, amino, carbamoyl, sulfamoyl, (1-6C)alkyl, (2-6C)alkenyl, (2-6C)alkynyl, (1-6C)alkoxy, (2-6C)alkenyloxy, (2-6C)alkynyloxy, (1-6C)alkylthio, (1-6C)alkylsulfinyl, (1-6C)alkylsulfonyl, (1-6C)alkylamino, (2-8C)alkenylamino, (2-8C)alkynylamino, di-[(1-6C)alkyl]amino, (2-6C)alkanoyl, N-(1-6C)alkylcarbamoyl, N,N-di-[(1-6C)alkyl]carbamoyl, (1-6C)alkoxycarbonyl, (2-6C)alkanoyloxy, (2-6C)alkanoylamino, N-(1-6C)alkyl-(2-6C)alkanoylamino, N-(1-6C)alkylsulfamoyl, N,N-di-[(1-6C)alkyl]sulfamoyl, (1-6C)alkanesulfonylamino and N-(1-6C)alkyl-(1-6C)alkanesulfonylamino

or from a group of the formula:
—X⁸—R³²
wherein X⁸ is a direct bond or is selected from O, C(O) and N(R³³), wherein R³³ is hydrogen or (1-6C)alkyl, and R³² is halo-(1-6C)alkyl, hydroxy-(1-6C)alkyl, (1-6C)alkoxy-(1-6C)alkyl, amino-(1-6C)alkyl, (1-6C)alkylamino-(1-6C)alkyl, di-[(1-6C)alkyl]amino-(1-6C)alkyl, (2-6C)alkanoylamino-(1-6C)alkyl and (1-6C)alkoxycarbonylamino-(1-6C)alkyl,

or from a group of the formula:
—X⁹-Q⁶
wherein X⁹ is a direct bond or is selected from O, S, SO, SO₂, C(O), N(R³⁴), C(O)N(R³⁴), N(R³⁴)C(O), SO₂N(R³⁴), N(R³⁴)SO₂ wherein R³⁴ is hydrogen or (1-6C)alkyl, and Q⁶ is aryl, aryl-(1-6C)alkyl, (3-7C)cycloalkyl, (3-7C)cycloalkyl-(1-6C)alkyl, heteroaryl, heteroaryl-(1-6C)alkyl, heterocyclyl or heterocyclyl-(1-6C)alkyl,

and wherein R³¹ optionally bears on carbon one or more R³⁶,

and wherein if any heteroaryl or heterocyclyl group within R³¹ contains an —NH-moiety, the nitrogen of said moiety optionally bears a group selected from R³⁷,

and wherein any heterocyclyl group within a substituent on R³¹ optionally bears 1 or 2 oxo or thioxo substituents;

R³⁵ and R³⁷ are each independently selected from (1-6C)alkyl, (2-8C)alkenyl, (2-8C)alkynyl, (1-6C)alkylsulfonyl and (2-6C)alkanoyl,

or from a group of the formula:
—X¹⁰-Q⁷
wherein X¹⁰ is a direct bond or is selected from C(O) and SO₂, wherein Q⁷ is (3-7C)cycloalkyl or (3-7C)cycloalkyl-(1-6C)alkyl;

R³⁶ is selected from halo, cyano, hydroxy, amino, (1-6C)alkyl, (2-6C)alkenyl, (2-6C)alkynyl, (3-6)cycloalkyl, (1-6C)alkoxy, (1-6C)alkylamino and di-[(1-6C)alkyl]amino,

provided that the compound of formula I is not 4-{[(2R)-2-amino-3-(3-pyridyl)-1-oxopropyl]amino}-N-[(2-ethyl-6-methylphenyl)thioxomethyl]-L-phenylalanine methyl ester;

or a pharmaceutically acceptable salt or prodrug thereof.

In a particular embodiment in formula (1), R¹ is selected from chloro, (1-3C)alkyl and halo-(1-3C)alkyl;

R⁴ is selected from hydrogen, (1-6C)alkyl, aryl, aryl-(1-6C)alkyl, heteroaryl and heteroaryl-(1-6C)alkyl, which optionally bears on carbon one or more R²¹ substituents, which may be the same or different,

and wherein if any heteroaryl group within R⁴ contains an —NH— moiety, the nitrogen of said moiety optionally bears a group selected from R²²,

X^{a} in formula I above is oxygen;

R⁶ is heteroaryl, which heteroaryl contains at least one nitrogen atom, and wherein R⁶ optionally bears on carbon one or more R³¹ substituents, and wherein if R⁶ contains an —NH— moiety, the nitrogen of said moiety optionally bears a group selected from R³⁵;

R³¹ is selected from halo, cyano, hydroxy, amino, (1-6C)alkyl, (2-6C)alkenyl, (2-6C)alkynyl, (1-6C)alkoxy, (1-6C)alkylamino and di-[(1-6C)alkyl]amino,

or from a group of the formula:
—X⁸—R³²
wherein X⁸ is a direct bond or is selected from O and N(R³³), wherein R³³ is hydrogen or (1-6C)alkyl, and R³² is halo-(1-6C)alkyl, hydroxy-(1-6C)alkyl, (1-6C)alkoxy-(1-6C)alkyl, amino-(1-6C)alkyl, (1-6C)alkylamino-(1-6C)alkyl and di-[(1-6C)alkyl]amino-(1-6C)alkyl,

or from a group of the formula:
—X⁹-Q⁶
wherein X⁹ is a direct bond or is selected from O and N(R³⁴), wherein R³⁴ is hydrogen or (1-6C)alkyl, and Q⁶ (3-7C)cycloalkyl or (3-7C)cycloalkyl-(1-6C)alkyl;

R³⁵ is selected from (1-6C)alkyl, (2-8C)alkenyl, (2-8C)alkynyl, (1-6C)alkylsulfonyl and (2-6C)alkanoyl,

or from a group of the formula:
—X¹⁰-Q⁷

wherein X¹⁰ is a direct bond or is selected from C(O) and SO₂, wherein Q⁷ is (3-7C)cycloalkyl or (3-7C)cycloalkyl-(1-6C)alkyl;

and

R², R³, R⁵, R²¹, R²², A, X, Y, Z, m and n are as hereinbefore defined.

In a particular embodiment in formula I,

R¹ is selected from chloro, (1-3C)alkyl and halo-(1-3C)alkyl;

R⁴ is selected from hydrogen, (1-6C)alkyl, aryl, aryl-(1-6C)alkyl, heteroaryl and heteroaryl-(1-6C)alkyl, which optionally bears on carbon one or more R²¹ substituents, which may be the same or different,

and wherein if any heteroaryl group within R⁴ contains an —NH— moiety, the nitrogen of said moiety optionally bears a group selected from R²²;

R⁶ is heteroaryl, which heteroaryl contains at least one nitrogen atom, and wherein R⁶ optionally bears on carbon one or more R³¹ substituents,

and wherein if R⁶ contains an —NH— moiety, the nitrogen of said moiety optionally bears a group selected from R³⁵;

R³¹ is selected from halo, cyano, hydroxy, amino, (1-6C)alkyl, (2-6C)alkenyl, (2-6C)alkynyl, (1-6C)alkoxy, (1-6C)alkylamino and di-[(1-6C)alkyl]amino,

or from a group of the formula:
—X⁸—R³²
wherein X⁸ is a direct bond or is selected from O and N(R³³), wherein R³³ is hydrogen or (1-6C)alkyl, and R³² is halo-(1-6C)alkyl, hydroxy-(1-6C)alkyl, (1-6C)alkoxy-(1-6C)alkyl, amino-(1-6C)alkyl, (1-6C)alkylamino-(1-6C)alkyl and di-[(1-6C)alkyl]amino-(1-6C)alkyl,

or from a group of the formula:
—X⁹-Q⁶
wherein X⁹ is a direct bond or is selected from O and N(R³⁴), wherein R³⁴ is hydrogen or (1-6C)alkyl, and Q⁶ (3-7C)cycloalkyl or (3-7C)cycloalkyl-(1-6C)alkyl;

R³⁵ is selected from (1-6C)alkyl, (2-8C)alkenyl, (2-8C)alkynyl, (1-6C)alkylsulfonyl and (2-6C)alkanoyl,

or from a group of the formula:
—X¹⁰-Q⁷

wherein X¹⁰ is a direct bond or is selected from C(O) and SO₂, wherein Q⁷ is (3-7C)cycloalkyl or (3-7C)cycloalkyl-(1-6C)alkyl; and

R², R³, R⁵, R²¹, R²², A, X, X^{a}, Y, Z, m and n are as hereinbefore defined.

Thus examples of compound of formula I include compounds of formula (I′)

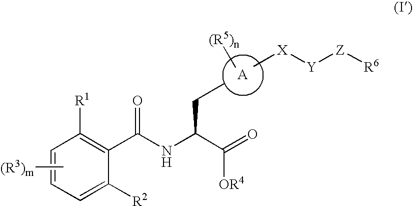

where R¹, R², R³, R⁴, R⁵, R⁶, A, X, Y, Z, m and n are as defined above.

In an embodiment of the invention there is provided a compound of the formula I which is of the formula IA:

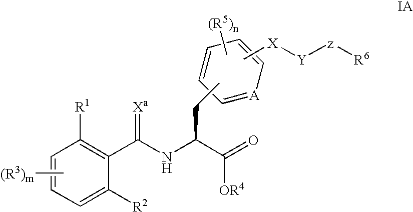

wherein:

A is N or CH, and n, m, R¹R², R³R⁴, R⁵R⁶, X^{a}, X, Y and Z are as hereinbefore defined;

or a pharmaceutically acceptable salt or prodrug thereof. In compounds of formula (IA′), X^{a} is oxygen.

In another embodiment of the invention there is provided a compound of the formula I which is of the formula IB:

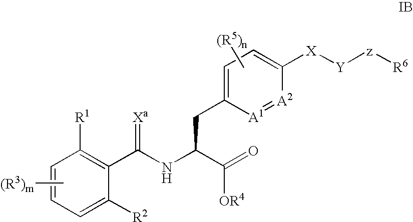

wherein:

A¹ and A² are selected from N and CH, provided that A¹ and A² are not both N;

and n, m, R¹, R², R³, R⁴, R⁵, R⁶, X^{a}, X, Y and Z are as hereinbefore defined;

or a pharmaceutically acceptable salt or prodrug thereof.

Compounds of formula (IB′) are compounds of formula (IB) wherein X^{a} is oxygen.

In another embodiment of the invention there is provided a compound of the formula I which is of the formula IC:

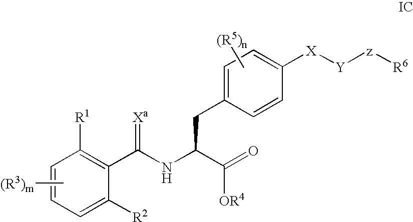

wherein:

n, m, R¹, R², R³, R⁴, R⁵, R⁶, X^{a}, X, Y and Z are as hereinbefore defined;

or a pharmaceutically acceptable salt or prodrug thereof.

Compounds of formula (IC′) are compounds of formula (IC) above where X^{a} is oxygen.

In another embodiment of the invention there is provided a compound of the formula I which is of the formula ID:

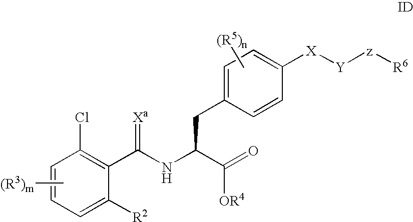

wherein:

R² is selected from hydrogen, fluoro, chloro, methyl and ethyl; and

n, m, R³, R⁴, R⁵, R⁶, X^{a}, X, Y and Z are as hereinbefore defined;

or a pharmaceutically acceptable salt or prodrug thereof.

Compounds of formula (ID′) are compounds of formula (ID) above where X^{a} is oxygen.

In another embodiment of the invention there is provided a compound of the formula I which is of the formula IE:

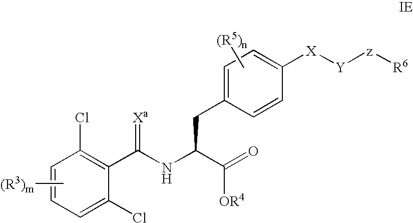

wherein:

X is selected from a direct bond, NR²⁶, O, S, SO, SO₂, C(O), CH(OR²⁶), C(O)N(R²⁶), N(R²⁶)C(O), SO₂N(R²⁶), N(R²⁶)SO₂, CH═CH and C≡C, wherein R²⁶ is hydrogen, (1-6C)alkyl or (3-7C)cycloalkyl;

Y is (1-6C)alkylene,

and wherein adjacent carbon atoms in any (2-6C)alkylene chain within a Y substituent are optionally separated by the insertion into the chain of a group selected from O, S, SO, SO₂, N(R²⁷), C(O), CH(OR²⁷), C(O)N(R²⁷), N(R²⁷)C(O), SO₂N(R²⁷), N(R²⁷)SO₂, CH═CH and C≡C wherein R²⁷ is hydrogen, (1-6C)alkyl or (3-7C)cycloalkyl;

Z is selected from a direct bond, NR²⁶, O, S, SO, SO₂, C(O), CH(OR²⁶), SO₂N(R²⁶), N(R²⁶)SO₂, CH═CH and C≡C, wherein R²⁶ is hydrogen, (1-6C)alkyl or (3-7C)cycloalkyl;

and wherein any X, Y or Z optionally bears on carbon one or more R²⁸ substituents;

and wherein n, m, X^{a}, R³, R⁴, R⁵, R⁶, and R²⁸ are as hereinbefore defined;

or a pharmaceutically acceptable salt or prodrug thereof.

Compounds of formula (IE′) are compounds of formula (IE) where X^{a} is oxygen.

In another embodiment of the invention there is provided a compound of the formula I which is of the formula IF:

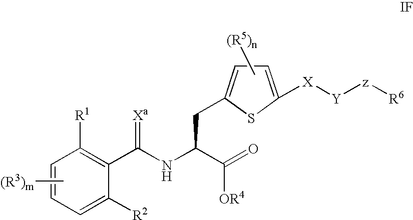

wherein n, m, R¹, R², R³, R⁴, R⁵, R⁶, X^{a}, X, Y and Z are as hereinbefore defined;

or a pharmaceutically acceptable salt or prodrug thereof.

Compounds of formula (IF′) are compounds of formula (IF) where X^{a} is oxygen.

Particular compounds of the formula I, (IA), (IB), (IC), (ID), (IE) and (IF) are those wherein:

R⁶ is heteroaryl, which heteroaryl contains at least one —N═ ring atom,

wherein R⁶ is linked to the group Z by a carbon atom in R⁶,

and wherein R⁶ optionally bears on carbon one or more R³¹ substituents,

and wherein if R⁶ contains an —NH— moiety, the nitrogen of said moiety optionally bears a group selected from R³⁵,

and wherein:

(i) R⁶ is a bicyclic or polycyclic heteroaryl which contains at least one unsubstituted —NH— ring member in addition to the —N═ ring atom, wherein the —NH— and ═N— group in R⁶ are attached to the same bridgehead ring atom at a junction of two fused rings in R⁶; or

(ii) R⁶ is substituted in an ortho position to the —N═ atom in R⁶ by an —NHR^{31a} group; or

(iii) Z is NH and R⁶ is attached to Z by a ring carbon atom in an ortho position to the —N═ atom in R⁶;

and wherein the group Z-R⁶ has a pKa of greater than or equal to about 6;

R^{31a} is selected from hydrogen, (1-6C)alkyl, (2-8C)alkenyl, (2-8C)alkynyl,

halo-(1-6C)alkyl, hydroxy-(2-6C)alkyl, (1-6C)alkoxy-(2-6C)alkyl, amino-(2-6C)alkyl, (1-6C)alkylamino-(2-6C)alkyl, di-[(1-6C)alkyl]amino-(2-6C)alkyl, (3-7C)cycloalkyl and (3-7C)cycloalkyl-(1-6C)alkyl,

and wherein any (3-7C)cycloalkyl in R^{31a} optionally bears 1 or more (1-6C)alkyl substituents; and

R³¹ and R³⁵ are as hereinbefore defined;

or a pharmaceutically acceptable salt or prodrug thereof.

Other particular compounds of the formula I, (IA), (IB), (IC), (ID), (IE) and (IF) are those wherein:

X^{a} is selected from oxygen or sulphur;

R¹ is selected from bromo, chloro, (1-3C)alkyl, cyclopropyl, cyclobutyl, cyclopentyl, cyclopropylmethyl, cyclobutylmethyl, cyclopentylmethyl and halo-(1-3C)alkyl;

R² and each R³, which may be the same or different, is selected from hydrogen, halo, trifluoromethyl, cyano, isocyano, nitro, hydroxy, mercapto, amino, formyl, carboxy, carbamoyl, sulfamoyl, (1-6C)alkyl, (2-8C)alkenyl, (2-8C)alkynyl, (1-6C)alkoxy, (2-6C)alkenyloxy, (2-6C)alkynyloxy, (1-6C)alkylthio, (1-6C)alkylsulfinyl, (1-6C)alkylsulfonyl, (1-6C)alkylamino, di-[(1-6C)alkyl]amino, (1-6C)alkoxycarbonyl, N-(1-6C)alkylcarbamoyl, N,N-di-[(1-6C)alkyl]carbamoyl, (2-6C)alkanoyl, (2-6C)alkanoyloxy, (2-6C)alkanoylamino, N-(1-6C)alkyl-(2-6C)alkanoylamino, (3-6C)alkenoylamino, N-(1-6C)alkyl-(3-6C)alkenoylamino, (3-6C)alkynoylamino, N-(1-6C)alkyl-(3-6C)alkynoylamino, N-(1-6C)alkylsulfamoyl, N,N-di-[(1-6C)alkyl]sulfamoyl, (1-6C)alkanesulfonylamino and N-(1-6C)alkyl-(1-6C)alkanesulfonylamino,

or from a group of the formula:
Q¹-X¹—
wherein X¹ is a direct bond or is selected from O, S, SO, SO₂, N(R⁷), C(O), CH(OR⁷), C(O)N(R⁷), N(R⁷)C(O), SO₂N(R⁷), N(R⁷)SO₂, OC(R⁷)₂, SC(R⁷)₂ and N(R⁷)C(R⁷)₂, wherein R⁷ is hydrogen or (1-6C)alkyl, and Q¹ is aryl, aryl-(1-6C)alkyl, (3-7C)cycloalkyl, (3-7C)cycloalkyl-(1-6C)alkyl, (3-7C)cycloalkenyl, (3-7C)cycloalkenyl-(1-6C)alkyl, heteroaryl, heteroaryl-(1-6C)alkyl, heterocyclyl or heterocyclyl-(1-6C)alkyl,

and wherein R² and any R³ independently of each other optionally bears on carbon one or more R⁸ groups,

and wherein if any heteroaryl or heterocyclyl group within R² and any R³ contains an —NH— moiety, the nitrogen of said moiety optionally bears a group selected from R⁹,

and wherein any heterocyclyl group within R² and any R³ optionally bears 1 or 2 oxo or thioxo substituents;

or R² and an R³ substituent optionally form a (1-3C)alkylenedioxy group;

or two R³ substituents optionally form a (1-3C)alkylenedioxy group;

m is 0, 1, 2 or 3;

R⁴ is selected from hydrogen, (1-6C)alkyl, heterocyclyl, heterocyclyl-(1-6C)alkyl, aryl, aryl-(1-6C)alkyl, heteroaryl and heteroaryl-(1-6C)alkyl, which optionally bears on carbon one or more R²¹ substituents, which may be the same or different,

and wherein if any heteroaryl group within R⁴ contains an —NH— moiety, the nitrogen of said moiety optionally bears a group selected from R²²,

and wherein and wherein any heterocyclyl group within R⁴ optionally bears 1 or 2 oxo or thioxo substituents; 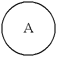
is selected from phenyl, pyridinyl and thiophenyl;

n is 0, 1, 2, 3 or 4, provided that when ring A is thiophenyl, n is 0, 1 or 2;

each R⁵, which may be the same or different, is selected from halo, trifluoromethyl, cyano, nitro, mercapto, amino, formyl, carboxy, carbamoyl, sulfamoyl, (1-6C)alkyl, (2-8C)alkenyl, (2-8C)alkynyl, (1-6C)alkoxy, (2-6C)alkenyloxy, (2-6C)alkynyloxy, (1-6C)alkylthio, (1-6C)alkylsulfinyl, (1-6C)alkylsulfonyl, (1-6C)alkylamino, di-[(1-6C)alkyl]amino, (1-6C)alkoxycarbonyl, N-(1-6C)alkylcarbamoyl, N,N-di-[(1-6C)alkyl]carbamoyl, (2-6C)alkanoyl, (2-6C)alkanoylamino, N-(1-6C)alkyl-(2-6C)alkanoylamino, (3-6C)alkenoylamino, N-(1-6C)alkyl-(3-6C)alkenoylamino, (3-6C)alkynoylamino, N-(1-6C)alkyl-(3-6C)alkynoylamino, N-(1-6C)alkylsulfamoyl, N,N-di-[(1-6C)alkyl]sulfamoyl, (1-6C)alkanesulfonylamino and N-(1-6C)alkyl-(1-6C)alkanesulfonylamino,

and wherein R⁵ optionally bears on carbon one or more R²⁴ groups selected from halo, cyano, hydroxy, carboxy, amino, (3-6C)cycloalkyl, (2-6C)alkenyl, (2-6C)alkynyl, (1-6C)alkoxy, (1-6C)alkylamino and di-[(1-6C)alkyl]amino,

or two R⁵ substituents optionally form a (1-3C)alkylenedioxy group;

X and Z, which may be the same or different, are selected from a direct bond, N(R²⁶), O, S, SO, SO₂, CH═CH and C≡C, wherein R²⁶ is hydrogen, (1-6C)alkyl or (3-7C)cycloalkyl;

Y is (1-4C)alkylene;

and wherein adjacent carbon atoms in any (2-4C)alkylene chain within a Y substituent are optionally separated by the insertion into the chain of a group selected from O, S, SO, SO₂, N(R²⁷), CH═CH and C≡C wherein R²⁷ is hydrogen, (1-6C)alkyl or (3-7C)cycloalkyl;

and wherein any X, Y or Z optionally bears on carbon one or more substituents selected from and R²⁸, wherein R²⁸ is selected from halo, cyano, hydroxy, amino, (1-4C)alkyl, (3-6C)cycloalkyl, (2-6C)alkenyl, (2-6C)alkynyl, (1-6C)alkoxy, (1-6C)alkylamino and di-[(1-6C)alkyl]amino;

R⁶ is heteroaryl, which heteroaryl contains at least one —N═ ring atom,

wherein R⁶ is linked to the group Z by a carbon atom in R⁶,

and wherein R⁶ optionally bears on carbon one or more R³¹ substituents,

and wherein if R⁶ contains an —NH— moiety, the nitrogen of said moiety optionally bears a group selected from R³⁵,

and wherein:

(i) R⁶ is a bicyclic or polycyclic heteroaryl which contains at least one unsubstituted —NH— ring member in addition to the —N═ ring atom, wherein the —NH— and ═N— group in R⁶ are attached to the same bridgehead ring atom at a junction of two fused rings in R⁶; or

(ii) R⁶ is substituted in an ortho position to the —N═ atom in R⁶ by an —NHR^{31a} group; or

(iii) Z is NH and R⁶ is attached to Z by a ring carbon atom in an ortho position to the —N═ atom in R⁶;

and wherein the group Z-R⁶ has a pKa of greater than or equal to about 6;

R⁸ and R²¹ is selected from halo, trifluoromethyl, cyano, nitro, hydroxy, amino, carboxy, carbamoyl, sulfamoyl, (1-6C)alkyl, (2-8C)alkenyl, (2-8C)alkynyl, (1-6C)alkoxy, (2-6C)alkenyloxy, (2-6C)alkynyloxy, (1-6C)alkylthio, (1-6C)alkylsulfinyl, (1-6C)alkylsulfonyl, (1-6C)alkylamino, di-[(1-6C)alkyl]amino, (1-6C)alkoxycarbonyl, N-(1-6C)alkylcarbamoyl, N,N-di-[(1-6C)alkyl]carbamoyl, (2-6C)alkanoyl, (2-6C)alkanoyloxy, (2-6C)alkanoylamino, N-(1-6C)alkyl-(2-6C)alkanoylamino, N-(1-6C)alkylsulfamoyl, N,N-di-[(1-6C)alkyl]sulfamoyl, (1-6C)alkanesulfonylamino and N-(1-6C)alkyl-(1-6C)alkanesulfonylamino,

or from a group of the formula:
—X²—R¹⁰
wherein X² is a direct bond or is selected from O, C(O) and N(R¹¹), wherein R¹¹ is hydrogen or (1-6C)alkyl, and R¹⁰ is halo-(1-6C)alkyl, hydroxy-(1-6C)alkyl, (1-6C)alkoxy-(1-6C)alkyl, cyano-(1-6C)alkyl, amino-(1-6C)alkyl, (1-6C)alkylamino-(1-6C)alkyl, di-[(1-6C)alkyl]amino-(1-6C)alkyl, (2-6C)alkanoylamino-(1-6C)alkyl and (1-6C)alkoxycarbonylamino-(1-6C)alkyl,

or from a group of the formula:
—X³-Q²

wherein X³ is a direct bond or is selected from O, S, SO, SO₂, N(R¹²), C(O), CH(OR¹²), C(O)N(R¹²), N(R¹²)C(O), SO₂N(R¹²), N(R¹²)SO₂, OC(R¹²)₂, SC(R¹²)₂ and N(R¹²)C(R¹²)₂, wherein R¹² is hydrogen or (1-6C)alkyl, and Q² is aryl, aryl-(1-6C)alkyl, (3-7C)cycloalkyl, (3-7C)cycloalkyl-(1-6C)alkyl, (3-7C)cycloalkenyl, (3-7C)cycloalkenyl-(1-6C)alkyl, heteroaryl, heteroaryl-(1-6C)alkyl, heterocyclyl or heterocyclyl-(1-6C)alkyl,

and wherein R⁸ and R²¹ independently of each other optionally bears on carbon one or more R¹³

and wherein if any heteroaryl or heterocyclyl group within R⁸ and R²¹ contains an —NH— moiety, the nitrogen of said moiety optionally bears a group selected from R¹⁴,

and wherein any heterocyclyl group within a substituent on R⁸ and R²¹ independently of each other optionally bears 1 or 2 oxo or thioxo substituents;

R⁹ and R²² are each independently selected from cyano, hydroxy, carboxy, carbamoyl, sulfamoyl, (1-6C)alkyl, (2-8C)alkenyl, (2-8C)alkynyl, (1-6C)alkylsulfonyl, (1-6C)alkoxycarbonyl, N-(1-6C)alkylcarbamoyl, N,N-di-[(1-6C)alkyl]carbamoyl, (2-6C)alkanoyl, N-(1-6C)alkylsulfamoyl and N,N-di-[(1-6C)alkyl]sulfamoyl,

or from a group of the formula:
—X⁴—R¹⁵
wherein X⁴ is a direct bond or is selected from C(O), SO₂, C(O)N(R¹⁶) and SO₂N(R¹⁶), wherein R¹⁶ is hydrogen or (1-6C)alkyl, and R¹⁵ is halo-(1-6C)alkyl, hydroxy-(1-6C)alkyl, (1-6C)alkoxy-(1-6C)alkyl, cyano-(1-6C)alkyl, amino-(1-6C)alkyl, (1-6C)alkylamino-(1-6C)alkyl, di-[(1-6C)alkyl]amino-(1-6C)alkyl, (2-6C)alkanoylamino-(1-6C)alkyl and (1-6C)alkoxycarbonylamino-(1-6C)alkyl,

or from a group of the formula:
—X⁵-Q³
wherein X⁵ is a direct bond or is selected from C(O), SO₂, C(O)N(R¹⁷) and SO₂N(R¹⁷), wherein R¹⁷ is hydrogen or (1-6C)alkyl, and Q³ is aryl, aryl-(1-6C)alkyl, (3-7C)cycloalkyl, (3-7C)cycloalkyl-(1-6C)alkyl, (3-7C)cycloalkenyl, (3-7C)cycloalkenyl-(1-6C)alkyl, heteroaryl, heteroaryl-(1-6C)alkyl, heterocyclyl or heterocyclyl-(1-6C)alkyl,

and wherein R⁹ and R²² independently of each other optionally bears on carbon one or more R¹⁸,

and wherein if any heteroaryl or heterocyclyl group within R⁹ and R²² contains an —NH— moiety, the nitrogen of said moiety optionally bears a group selected from R¹⁹,

and wherein any heterocyclyl group within a substituent on R⁹ and R²² optionally bears 1 or 2 oxo or thioxo substituents;

R¹³ and R¹⁸ are each independently selected from halo, cyano, hydroxy, carboxy, amino, (3-6C)cycloalkyl, (2-6C)alkenyl, (2-6C)alkynyl, (1-6C)alkoxy, (1-6C)alkylamino and di-[(1-6C)alkyl]amino;

R¹⁴ and R¹⁹ are each independently selected from carbamoyl, sulfamoyl, (1-6C)alkyl, (2-8C)alkenyl, (2-8C)alkynyl, (1-6C)alkylsulfonyl, N-(1-6C)alkylcarbamoyl, N,N-di-[(1-6C)alkyl]carbamoyl, (2-6C)alkanoyl, N-(1-6C)alkylsulfamoyl and N,N-di-[(1-6C)alkyl]sulfamoyl,

or from a group of the formula:
—X⁶-Q⁴

wherein X⁶ is a direct bond or is selected from C(O), SO₂, C(O)N(R²⁰) and SO₂N(R²⁰), wherein R²⁰ is hydrogen or (1-6C)alkyl, and Q⁴ is (3-7C)cycloalkyl or (3-7C)cycloalkyl-(1-6C)alkyl;

R³¹ is selected from halo, cyano, hydroxy, nitro, amino, carbamoyl, sulfamoyl, (1-6C)alkyl, (2-6C)alkenyl, (2-6C)alkynyl, (1-6C)alkoxy, (2-6C)alkenyloxy, (2-6C)alkynyloxy, (1-6C)alkylthio, (1-6C)alkylsulfinyl, (1-6C)alkylsulfonyl, (1-6C)alkylamino, di-[(1-6C)alkyl]amino, (2-6C)alkanoyl, N-(1-6C)alkylcarbamoyl, N,N-di-[(1-6C)alkyl]carbamoyl, (1-6C)alkoxycarbonyl, (2-6C)alkanoyloxy, (2-6C)alkanoylamino, N-(1-6C)alkyl-(2-6C)alkanoylamino, N-(1-6C)alkylsulfamoyl, N,N-di-[(1-6C)alkyl]sulfamoyl, (1-6C)alkanesulfonylamino and N-(1-6C)alkyl-(1-6C)alkanesulfonylamino

or from a group of the formula:
—X⁸—R³²
wherein X⁸ is a direct bond or is selected from O, C(O) and N(R³³), wherein R³³ is hydrogen or (1-6C)alkyl, and R³² is halo-(1-6C)alkyl, hydroxy-(1-6C)alkyl, (1-6C)alkoxy-(1-6C)alkyl, amino-(1-6C)alkyl, (1-6C)alkylamino-(1-6C)alkyl, di-[(1-6C)alkyl]amino-(1-6C)alkyl, (2-6C)alkanoylamino-(1-6C)alkyl and (1-6C)alkoxycarbonylamino-(1-6C)alkyl,

or from a group of the formula:
—X⁹-Q⁶
wherein X⁹ is a direct bond or is selected from O, S, SO, SO₂, C(O), N(R³⁴), C(O)N(R³⁴), N(R³⁴)C(O), SO₂N(R³⁴), N(R³⁴)SO₂ wherein R³⁴ is hydrogen or (1-6C)alkyl, and Q⁶ is aryl, aryl-(1-6C)alkyl, (3-7C)cycloalkyl, (3-7C)cycloalkyl-(1-6C)alkyl, heteroaryl, heteroaryl-(1-6C)alkyl, heterocyclyl or heterocyclyl-(1-6C)alkyl,

and wherein R³¹ optionally bears on carbon one or more R³⁶,

and wherein if any heteroaryl or heterocyclyl group within R³¹ contains an —NH— moiety, the nitrogen of said moiety optionally bears a group selected from R³⁷,

and wherein any heterocyclyl group within a substituent on R³¹ optionally bears 1 or 2 oxo or thioxo substituents;

R^{31a} is selected from hydrogen, (1-6C)alkyl, (2-8C)alkenyl, (2-8C)alkynyl,

halo-(1-6C)alkyl, hydroxy-(2-6C)alkyl, (1-6C)alkoxy-(2-6C)alkyl, amino-(2-6C)alkyl, (1-6C)alkylamino-(2-6C)alkyl, di-[(1-6C)alkyl]amino-(2-6C)alkyl, (3-7C)cycloalkyl and (3-7C)cycloalkyl-(1-6C)alkyl,

and wherein any (3-7C)cycloalkyl in R^{31a} optionally bears 1 or more (1-6C)alkyl substituents;

R³⁵ and R³⁷ are each independently selected from (1-6C)alkyl, (2-8C)alkenyl, (2-8C)alkynyl, (1-6C)alkylsulfonyl and (2-6C)alkanoyl,

or from a group of the formula:
—X¹⁰-Q⁷
wherein X¹⁰ is a direct bond or is selected from C(O) and SO₂, wherein Q⁷ is (3-7C)cycloalkyl or (3-7C)cycloalkyl-(1-6C)alkyl;

R³⁶ is selected from halo, cyano, hydroxy, amino, (1-6C)alkyl, (2-6C)alkenyl, (2-6C)alkynyl, (3-6)cycloalkyl, (1-6C)alkoxy, (1-6C)alkylamino and di-[(1-6C)alkyl]amino,

or a pharmaceutically acceptable salt or prodrug thereof.

Other particular compounds of the formula I, (IA), (IB), (IC), (ID), (IE) and (IF) are those wherein R⁶ is linked to Z by a carbon atom in an aromatic ring in R⁶ and wherein R⁶ is selected from any one of (a) to (f):

(a) imidazole which is substituted in an ortho position to the —N═ of the imidazole ring by —NHR^{31a};

(b) imidazole fused to: (bi) a monocyclic 6-membered aromatic, (bii) a monocyclic 5- or 6-membered heteroaromatic, (biii) a 3 to 7-membered heterocyclic or (biv) a (3-6C)cycloalkane ring,

and wherein R⁶ is substituted in the ortho position to the —N═ of the imidazole ring by —NHR^{31a};

(c) imidazole fused to a 5-, 6 or 7-membered heterocyclic, or to a monocyclic 5- or 6-membered heteroaromatic which heterocyclic or heteroaromatic contains at least one unsubstituted —NH— ring member and optionally contains 1 or 2 additional hetero atoms selected from O, S and N, and wherein the unsubstituted —NH— of the heterocyclic or heteroaromatic ring and the ═N— of the imidazole in R⁶ are attached to the same bridgehead ring atom at a junction of the two fused rings;

(d) pyridine which is substituted in an ortho position to the —N═ of the pyridine ring by —NHR^{31a};

(e) pyridine fused to (bi) a monocyclic 6-membered aromatic, (bii) a monocyclic 5- or 6-membered heteroaromatic, (biii) a 3 to 7-membered heterocyclic or (biv) a (3-6C)cycloalkane ring,

and wherein R⁶ is substituted in an ortho position to the —N═ of the pyridine ring by —NHR^{31a}; and

(f) pyridine fused to a 5-, 6 or 7-membered heterocyclic, or to a monocyclic 5- or 6-membered heteroaromatic which heterocyclic or heteroaromatic contains at least one unsubstituted —NH— ring member and optionally contains 1 or 2 additional hetero atoms selected from O, S and N, and wherein the unsubstituted —NH— of the heterocyclic ring and the ═N— of the pyridine ring in R⁶ are attached to the same bridgehead ring atom at a junction of the two fused rings;

or the group Z is NH, R⁶ is attached to Z by a carbon atom in an aromatic ring in R⁶ that is ortho to a —N═ ring atom in R⁶ and wherein R⁶ is selected from any one of (i) to (iiii):

(i) imidazole;

(ii) imidazole fused to a 6-membered monocyclic aromatic, a 3- to 7-membered monocyclic heteroaromatic or heterocyclic ring or to a (3-6C)cycloalkane ring; and

(iii) pyridine fused 5- or 6-membered monocyclic aromatic, a 3- to 7-membered monocyclic heteroaromatic or heterocyclic ring or to a (3-6C)cycloalkane ring;

and wherein R⁶ optionally bears on carbon one or more R³¹ substituents,

and wherein if R⁶ contains an —NH— ring member, the nitrogen of said —NH— group optionally bears a group selected from R³⁵ (provided said —NH— group is not specified to be an unsubstituted —NH— above);

wherein R³¹, R^{31a} and R³⁵ are as defined in claim 1;

or a pharmaceutically acceptable salt or prodrug thereof.

Other particular compounds of the formula I, (IA), (IB), (IC), (ID), (IE) and (IF) are those wherein R⁶ is:

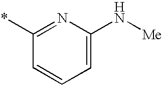

wherein * indicates the point of attachment of R⁶ to the group Z in formula I.

Other particular compounds of the formula I, (IA), (IB), (IC), (ID), (IE) and (IF) are those wherein R⁶ is:

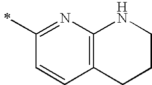

wherein * indicates the point of attachment of R⁶ to the group Z in formula I.

Other particular compounds of the formula I, according to the invention, such as (IA), (IB), (IC), (ID), (IE) and (IF) are those wherein the group R⁶-Z- in Formula I is not:

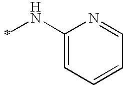

wherein * indicates the point of attachment of R⁶ to the group Z in formula I.

Other particular compounds of the formula I according to the invention, such as (IA), (IB), (IC), (ID), (IE) and (IF) are those wherein the group R⁶-Z-X—Y— in Formula I is not:

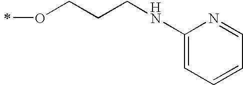

wherein * indicates the point of attachment to Ring A in formula I.

In a further embodiment of the invention the compound of formula I is not:
N-(2,6-dimethylbenzoyl)-O-[3-(pyridin-2-ylamino)propyl]-L-tyrosine;
N-(mesitylcarbonyl)-O-[3-(pyridin-2-ylamino)propyl]-L-tyrosine;
N-(mesitylsulfonyl)-O-[3-(pyridin-2-ylamino)propyl]-L-tyrosine; or
N-(4-isopropoxy-2,6-dimethylbenzoyl)-O-[3-(pyridin-2-ylamino)propyl]-L-tyrosine.

Also provided is a compound of formula I, or a pharmaceutically acceptable salt or prodrug thereof in association with a pharmaceutically acceptable carrier, diluent, or excipient.

Also provided is a compound of formula I, or a pharmaceutically acceptable salt or prodrug thereof, which is an a5b1 integrin antagonist useful for controlling pathologically angiogenic diseases, thrombosis, cardiac infarction, coronary heart diseases, arteriosclerosis, tumours, osteoporosis, inflammations or infections.

Also provided is a method of treating a disease or condition mediated by a5b1 which comprises administering to a patient in need of such treatment a compound of formula I, or a pharmaceutically acceptable salt or prodrug thereof.

Also provided is a process for the preparation of a compound of formula I as defined herein.

### DETAILED DESCRIPTION OF THE INVENTION

Unless otherwise stated, the following terms used in the specification and claims have the following meanings.

### DEFINITIONS

“Halo” means fluoro, chloro, bromo or iodo.

“(1-6C)alkyl” means a linear saturated monovalent hydrocarbon radical of one to six carbon atoms or a branched saturated monovalent hydrocarbon radical of three to six carbon atoms, for example methyl, ethyl, propyl, 2-propyl, tert-butyl, sec-butyl, n-pentyl, n-hexyl and the like.

An “alkylene.” “alkenylene,” or “alkynylene” group is an alkyl, alkenyl, or alkynyl group that is positioned between and serves to connect two other chemical groups. Thus, “(1-6C)alkylene” means a linear saturated divalent hydrocarbon radical of one to six carbon atoms or a branched saturated divalent hydrocarbon radical of three to six carbon atoms, for example, methylene, ethylene, propylene, 2-methylpropylene, pentylene, and the like.

“(2-6C)Alkenylene” means a linear divalent hydrocarbon radical of two to six carbon atoms or a branched divalent hydrocarbon radical of two to six carbon atoms, containing at least one double bond, for example, as in ethenylene, 2,4-pentadienylene, and the like.

“(2-6C)Alkynylene” means a linear divalent hydrocarbon radical of two to six carbon atoms or a branched divalent hydrocarbon radical of three to six carbon atoms, containing at least one triple bond, for example, as in ethynylene, propynylene, and butynylene and the like.

“(3-7C)Cycloalkyl” means a hydrocarbon ring containing from 3 to 7 carbon atoms, for example, cyclopropyl, cyclobutyl, cyclopentyl, cyclohexyl or bicyclo[2.2.1]heptyl

“(3-7C)Cycloalkyl” means a hydrocarbon ring containing at least one double bond, for example, cyclobutenyl, cyclopentenyl, cyclohexenyl or cycloheptenyl, such as 3-cyclohexen-1-yl.

“(3-7C)Cycloalkyl-(1-6C)alkylene” means a (3-7C)cycloalkyl group covalently attached to a (1-6C)alkylene group, both of which are defined herein.

The term “non-aromatic” refers to unsaturated ring systems without aromatic character, for example partially saturated and fully saturated carbocyclic and heterocyclic ring systems. The terms “unsaturated” and “partially saturated” refer to non-aromatic rings wherein the ring structure(s) contains atoms sharing more than one valence bond i.e. the ring contains at least one multiple bond e.g. a C═C, C≡C or N═C bond. The term “fully saturated” refers to rings where there are no multiple bonds between ring atoms. Saturated carbocyclic groups include, for example the cycloalkyl groups as defined herein. Partially saturated carbocyclic groups include cycloalkenyl groups as defined herein, for example cyclopentenyl, cycloheptenyl and cyclooctenyl. Partially saturated heterocyclyl rings include for example, dihydrothiophene, dihydrofuran, pyrroline, dihydropyran or tetrahydropyridine.

The term “heterocyclyl” means a non aromatic saturated or partially saturated monocyclic, fused, bridged, or spiro bicyclic heterocyclic ring system(s). Monocyclic heterocyclic rings contain from about 3 to 12 ring atoms, with from 1 to 5 heteroatoms selected from N, O, and S, and suitably from 3 to 7 member atoms, in the ring. Bicyclic heterocycles contain from 7 to 17 member atoms, suitably 7 to 12 member atoms, in the ring. Bicyclic heterocycles contain from about 7 to about 17 ring atoms, suitably from 7 to 12 ring atoms. Bicyclic heterocyclic(s) rings may be fused, spiro, or bridged ring systems. Examples of heterocyclic groups include cyclic ethers (oxiranes) such as ethyleneoxide, tetrahydrofuran, dioxane, and substituted cyclic ethers. Heterocycles containing nitrogen include, for example, azetidine, pyrrolidine, piperidine, piperazine, tetrahydrotriazine, tetrahydropyrazole, and the like. Typical sulfur containing heterocycles include tetrahydrothiophene, dihydro-1,3-dithiol-2-yl, and hexahydrothiepin-4-yl. Other heterocycles include dihydro-oxathiol-4-yl, tetrahydro-oxazolyl, tetrahydro-oxadiazolyl, tetrahydrodioxazolyl, tetrahydro-oxathiazolyl, hexahydrotriazinyl, tetrahydro-oxazinyl, morpholinyl, thiomorpholinyl, tetrahydropyrimidinyl, dioxolinyl, octahydrobenzofuranyl, octahydrobenzimidazolyl, and octahydrobenzothiazolyl. For heterocycles containing sulfur, the oxidized sulfur heterocycles containing SO or SO₂ groups are also included. Examples include the sulfoxide and sulfone forms of tetrahydrothiophene. A suitable value for a heterocyclyl group which bears 1 or 2 oxo or thioxo substituents is, for example, 2-oxopyrrolidinyl, 2-thioxopyrrolidinyl, 2-oxoimidazolidinyl, 2-thioxoimidazolidinyl, 2-oxopiperidinyl, 2,5-dioxopyrrolidinyl, 2,5-dioxoimidazolidinyl or 2,6-dioxopiperidinyl.

By “bridged ring systems” is meant ring systems in which two rings share more than two atoms, see for example Advanced Organic Chemistry, by Jerry March, 4^{th} Edition, Wiley Interscience, pages 131-133, 1992. Examples of bridged heterocyclyl ring systems include, aza-bicyclo[2.2.1]heptane, 2-oxa-5-azabicyclo[2.2.1]heptane, aza-bicyclo[2.2.2]octane, and aza-bicyclo[3.2.1]octane.

“Heterocyclyl-(1-6C)alkyl” means a heterocyclyl group covalently attached to a (1-6C)alkylene group, both of which are defined herein.

The term “aryl” means a cyclic or polycyclic aromatic ring having from 5 to 12 carbon atoms. The term aryl includes both monovalent species and divalent species. Examples of aryl groups include, but are not limited to, phenyl, biphenyl, naphthyl and the like.

“Aryl-(1-6C)alkyl” means an aryl group covalently attached to a (1-6C)alkyl group, both of which are defined herein. Examples of aryl-(1-6C)alkyl groups include benzyl, phenylethyl, and the like

The term “heteroaryl” means an aromatic mono-, bi-, or polycyclic ring incorporating one or more (for example 1-4) heteroatoms selected from N, O, and S. The term heteroaryl includes both monovalent species and divalent species. Examples of heteroaryl groups are monocyclic and bicyclic groups containing from five to twelve ring members, and more usually from five to ten ring members. The heteroaryl group can be, for example, a five membered or six membered monocyclic ring or a bicyclic structure formed from fused five and six membered rings or two fused six membered rings. Each ring may contain up to about four heteroatoms typically selected from nitrogen, sulphur and oxygen. Typically the heteroaryl ring will contain up to 3 heteroatoms, more usually up to 2, for example a single heteroatom. In one embodiment, the heteroaryl ring contains at least one ring nitrogen atom. The nitrogen atoms in the heteroaryl rings can be basic, as in the case of an imidazole or pyridine, or essentially non-basic as in the case of an indole or pyrrole nitrogen. In general the number of basic nitrogen atoms present in the heteroaryl group, including any amino group substituents of the ring, will be less than five.

Examples of heteroaryl include furyl, pyrrolyl, thienyl, oxazolyl, isoxazolyl, imidazolyl, pyrazolyl, thiazolyl, isothiazolyl, oxadiazolyl, thiadiazolyl, triazolyl, tetrazolyl, pyridyl, pyridazinyl, pyrimidinyl, pyrazinyl, 1,3,5-triazenyl, benzofuranyl, indolyl, isoindolyl, benzothienyl, benzoxazolyl, benzimidazolyl, benzothiazolyl, benzothiazolyl, indazolyl, purinyl, benzofurazanyl, quinolyl, isoquinolyl, quinazolinyl, quinoxalinyl, cinnolinyl, pteridinyl, naphthyridinyl, carbazolyl, phenazinyl, benzisoquinolinyl, pyridopyrazinyl, thieno[2,3-b]furanyl, 2H-furo[3,2-b]-pyranyl, 5H-pyrido[2,3-d]-o-oxazinyl, 1H-pyrazolo[4,3-d]-oxazolyl, 4H-imidazo[4,5-d]thiazolyl, pyrazino[2,3-d]pyridazinyl, imidazo[2,1-b]thiazolyl, imidazo[1,2-b][1,2,4]triazinyl. “Heteroaryl” also covers partially aromatic bicyclic and polycyclic ring systems wherein at least one ring is an aromatic ring and one or more of the other ring(s) is a non-aromatic, saturated or partially saturated ring, provided that at least one ring contains one or more (for example 1, 2 or 3) heteroatoms selected from O, S and N. For example a partially aromatic bicyclic ring may comprise 1 aromatic ring containing 1 or more heteroatoms selected from O, S and N and the other ring is a non-aromatic, saturated or partially unsaturated ring optionally containing one or more heteroatoms selected from O, S and N. Examples of partially aromatic heteroaryl rings include 1,2,3,4-tetrahydro-1,8-naphthyridinyl, 1,2,3,4-tetrahydropyrido[2,3-b]pyrazinyl and 3,4-dihydro-2H-pyrido[3,2-b][1,4]oxazinyl. References herein to a “6,5” or “6,6” aryl or heteroaryl ring systems refer to 5 membered ring fused to another 6 membered ring such as a benzothienyl ring (a 6,5 ring); or one 6 membered ring fused to another 6 membered ring such as a napthyl, quinolyl or quinazolinyl ring (a 6,6 ring). Unless stated otherwise, a 6,5 heteroaryl or aryl group may be attached via the 5 or the 6 membered ring.

Examples of five membered heteroaryl groups include but are not limited to pyrrole, furan, thiophene, imidazole, furazan, oxazole, oxadiazole, oxatriazole, isoxazole, thiazole, isothiazole, pyrazole, triazole and tetrazole groups.

Examples of six membered heteroaryl groups include but are not limited to pyridine, pyrazine, pyridazine, pyrimidine and triazine.

A bicyclic heteroaryl group may be, for example, a group selected from:

a) a benzene ring fused to a 5- or 6-membered ring containing 1, 2 or 3 ring heteroatoms;
b) a pyridine ring fused to a 5- or 6-membered ring containing 1, 2 or 3 ring heteroatoms;
c) a pyrimidine ring fused to a 5- or 6-membered ring containing 1 or 2 ring heteroatoms;
d) a pyrrole ring fused to a 5- or 6-membered ring containing 1, 2 or 3 ring heteroatoms;
e) a pyrazole ring fused to a 5- or 6-membered ring containing 1 or 2 ring heteroatoms;
f) a pyrazine ring fused to a 5- or 6-membered ring containing 1 or 2 ring heteroatoms;
g) an imidazole ring fused to a 5- or 6-membered ring containing 1 or 2 ring heteroatoms;
h) an oxazole ring fused to a 5- or 6-membered ring containing 1 or 2 ring heteroatoms;
i) an isoxazole ring fused to a 5- or 6-membered ring containing 1 or 2 ring heteroatoms;
j) a thiazole ring fused to a 5- or 6-membered ring containing 1 or 2 ring heteroatoms;
k) an isothiazole ring fused to a 5- or 6-membered ring containing 1 or 2 ring heteroatoms;
l) a thiophene ring fused to a 5- or 6-membered ring containing 1, 2 or 3 ring heteroatoms;
m) a furan ring fused to a 5- or 6-membered ring containing 1, 2 or 3 ring heteroatoms;
n) a cyclohexyl ring fused to a 5- or 6-membered ring containing 1, 2 or 3 ring heteroatoms; and
o) a cyclopentyl ring fused to a 5- or 6-membered heteroaryl ring containing 1, 2 or 3 ring heteroatoms.

Particular examples of bicyclic heteroaromatic groups containing a six membered ring fused to a five membered ring include but are not limited to benzfuran, benzthiophene, benzimidazole, benzoxazole, benzisoxazole, benzthiazole, benzisothiazole, isobenzofuran, indole, isoindole, indolizine, indoline, isoindoline, purine (e.g., adenine, guanine), indazole, benzodioxole and pyrazolopyridine groups.

Particular examples of bicyclic heteroaromatic groups containing two fused six membered rings include but are not limited to quinoline, isoquinoline, chroman, thiochroman, chromene, isochromene, isochroman, benzodioxan, quinolizine, benzoxazine, benzodiazine, pyridopyridine, quinoxaline, quinazoline, cinnoline, phthalazine, naphthyridine and pteridine groups.

“Heteroaryl-(1-6C)alkyl” means an heteroaryl group covalently attached to a (1-6C)alkyl group, both of which are defined herein. Examples of heteroaralkyl groups include pyridin-3-ylmethyl, 3-(benzofuran-2-yl)propyl, and the like.

“Haloalkyl” means alkyl substituted with one or more same or different halo atoms, e.g., —CH₂Cl, —CF₃, —CH₂CF₃, —CH₂CCl₃, and the like.

Examples for the substituents within the compound of formula I include:—
for halo fluoro, chloro, bromo and iodo;
for (1-6C)alkyl: methyl, ethyl, propyl, isopropyl and tert-butyl;
for (2-8C)alkenyl: vinyl, isopropenyl, allyl and but-2-enyl;
for (2-8C)alkynyl: ethynyl, 2-propynyl and but-2-ynyl;
for (1-6C)alkoxy: methoxy, ethoxy, propoxy, isopropoxy and butoxy;
for (2-6C)alkenyloxy: vinyloxy and allyloxy;
for (2-6C)alkynyloxy: ethynyloxy and 2-propynyloxy;
for (1-6C)alkylthio: methylthio, ethylthio and propylthio;
for (1-6C)alkylsulfinyl: methylsulfinyl and ethylsulfinyl;
for (1-6C)alkylsulfonyl: methylsulfonyl and ethylsulfonyl;
for (1-6C)alkylamino: methylamino, ethylamino, propylamino, isopropylamino and butylamino;
for di-[(1-6C)alkyl]amino: dimethylamino, diethylamino, N-ethyl-N-methylamino and diisopropylamino;
for (1-6C)alkoxycarbonyl: methoxycarbonyl, ethoxycarbonyl, propoxycarbonyl and tert-butoxycarbonyl;
for N-(1-6C)alkylcarbamoyl: N-methylcarbamoyl, N-ethylcarbamoyl and N-propylcarbamoyl;
for N,N-di-[(1-6C)alkyl]carbamoyl: N,N-dimethylcarbamoyl, N-ethyl-N-methylcarbamoyl and N,N-diethylcarbamoyl;
for (2-6C)alkanoyl: acetyl and propionyl;
for (2-6C)alkanoyloxy: acetoxy and propionyloxy;
for (2-6C)alkanoylamino: acetamido and propionamido;
for N-(1-6C)alkyl-(2-6C)alkanoylamino: N-methylacetamido and N-methylpropionamido;
for N-(1-6C)alkyl sulfamoyl: N-methylsulfamoyl and N-ethylsulfamoyl;
for N,N-di-[(1-6C)alkyl]sulfamoyl: N,N-dimethylsulfamoyl;
for (1-6C)alkanesulfonylamino: methanesulfonylamino and ethanesulfonylamino;
for N-(1-6C)alkyl-(1-6C)alkanesulfonylamino: N-methylmethanesulfonylamino and N-methylethanesulfonylamino;
for (3-6C)alkenoylamino: acrylamido, methacrylamido and crotonamido;
for N-(1-6C)alkyl-(3-6C)alkenoylamino: N-methylacrylamido and N-methylcrotonamido;
for (3-6C)alkynoylamino: propiolamido;
for N-(1-6C)alkyl-(3-6C)alkynoylamino: N-methylpropiolamido;
for amino-(1-6C)alkyl: aminomethyl, 2-aminoethyl, 1-aminoethyl and 3-aminopropyl;
for (1-6C)alkylamino-(1-6C)alkyl: methylaminomethyl, ethylaminomethyl, 1-methylaminoethyl, 2-methylaminoethyl, 2-ethylaminoethyl and 3-methylaminopropyl;
for di-[(1-6C)alkyl]amino-(1-6C)alkyl: dimethylaminomethyl, diethylaminomethyl, 1-dimethylaminoethyl, 2-dimethylaminoethyl and 3-dimethylaminopropyl;
for hydroxy-(1-6C)alkyl: hydroxymethyl, 2-hydroxyethyl, 1-hydroxyethyl and
   3-hydroxypropyl;
for (1-6C)alkoxy-(1-6C)alkyl: methoxymethyl, ethoxymethyl, 1-methoxyethyl, 2-methoxyethyl, 2-ethoxyethyl and 3-methoxypropyl;
for cyano-(1-6C)alkyl: cyanomethyl, 2-cyanoethyl, 1-cyanoethyl and 3-cyanopropyl;
for (1-6C)alkylthio-(1-6C)alkyl: methylthiomethyl, ethylthiomethyl, 2-methylthioethyl, 1-methylthioethyl and 3-methylthiopropyl;
for (1-6C)alkylsulfinyl-(1-6C)alkyl: methylsulfinylmethyl, ethylsulfinylmethyl, 2-methylsulfinylethyl, 1-methylsulfinylethyl and 3-methyl sulfinylpropyl;
for (1-6C)alkylsulfonyl-(1-6C)alkyl: methylsulfonylmethyl, ethylsulfonylmethyl, 2-methylsulfonylethyl, 1-methylsulfonylethyl and 3-methyl sulfonylpropyl;
for (2-6C)alkanoylamino-(1-6C)alkyl: acetamidomethyl, propionamidomethyl and 2-acetamidoethyl; and
for (1-6C)alkoxycarbonylamino-(1-6C)alkyl: methoxycarbonylaminomethyl, ethoxycarbonylaminomethyl, tert-butoxycarbonylaminomethyl and 2-methoxycarbonylaminoethyl.

The term “(1-3C)alkylenedioxy” includes for example, methylenedioxy or ethylenedioxy and the oxygen atoms thereof occupy adjacent ring positions. For example when R² and an adjacent R³ form a methylenedioxy group the amide/thioamide in formula I is of the formula:

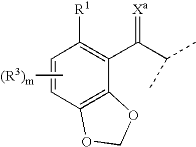

When, as defined hereinbefore, an R² group is a group of the formula Q¹-X¹— and, for example, X¹ is a OC(R⁷)₂ linking group, it is the carbon atom, not the oxygen atom, of the OC(R⁷)₂ linking group which is attached to the phenyl ring in formula I and the oxygen atom is attached to the Q¹ group. Similarly, when, for example R⁹ is a group of the formula —X⁴—R¹⁵ and, for example, X⁴ is a C(O)N(R¹⁶) linking group, it is the N(R¹⁶) group, not the carbonyl group of the C(O)N(R¹⁶) linking group which is attached to the R¹⁵ group. A similar convention applies to the attachment of the groups of the formulae “Q-X-” and “—X-Q” defined herein.

As defined hereinbefore, adjacent carbon atoms in any (2-6C)alkylene chain within for example an X, Y or Z group may be optionally separated by the insertion into the chain of a group such as O, C(O)N(R²⁶), N(R²⁶) or C≡C. For example, insertion of a C≡C group into the ethylene chain gives a but-2-ynylene group and, for example, insertion of a C(O)NH group into an ethylene chain gives rise to —CH₂C(O)NHCH₂—, similarly the insertion of an oxygen atom into a propylene chain gives for example —CH₂OCH₂CH₂—.

Where herein it is stated that the chain length of the group —X—Y-Z- is, for example 3 atoms, this means that the number of linked atoms between ring A and R⁶ is 3. For example, where —X—Y-Z- is: 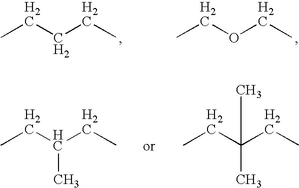
the chain length of —X—Y-Z- is 3 atoms. When Y is heterocyclyl, the chain length is the shortest linked chain between ring A and R⁶. Accordingly, when —X—Y-Z- is a group of the formula: 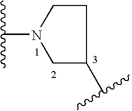
the chain length between ring A and R⁶ is 3 atoms and not 4 atoms.

Similarly, references herein to the chain length between ring A and a —N═ atom in R⁶, refer to the shortest chain length of the group —X—Y-Z- together with any ring atoms (including bridgehead atoms) in R⁶ up to and including said —N═ ring atom. By way of example, the chain length between ring A and the —N═ ring atom in R⁶ in each —X—Y-Z-R⁶ group shown below is 5: 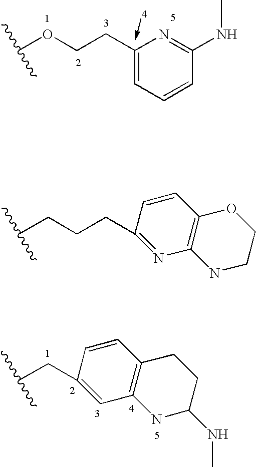
wherein [symbol] indicates the point of attachment of —X—Y-Z-R⁶ to ring A.

Reference herein to R⁶ containing a —N═ ring atom in a ring refer to, for example, the ring nitrogen of a pyridine. As will be clear, reference to a —N═ atom indicates that the nitrogen has 3 bonds to it which are part of the ring structure (as in a pyridine nitrogen) and a —N═ atom does not refer to a —NH— or substituted —NH— ring member (as in for example the NH of indole). Unless stated otherwise, R⁶ may be a mono-bicyclic or polycyclic heteroaryl ring system. It is to be understood that in addition to nitrogen R⁶ may also contain 1 or more additional heteroatoms (for example 1 to 4) selected from O, S and N and that these additional heteroatoms may be in the same ring or different rings to the —N=atom.

It is to be understood that where it is stated that R⁶ contains a ring —N═ group and R⁶ contains at least one “unsubstituted —NH— group” in the heteroaryl ring(s), said —NH— is in addition to the —N═ group and is not substituted by an R³⁵ group. In addition to said —N═ and —NH— group R⁶ may contain one or more additional heteroatoms selected from O, S and N.

It is also to be understood that when R⁶ is a bicyclic or polycyclic heteroaryl which contains at least one unsubstituted —NH— ring member in addition to a —N═ ring atom, wherein the —NH— and ═N— group in R⁶ are attached to the same bridgehead ring atom at a junction of two fused rings in R⁶; then the —N═ and —NH— ring members are located in the ortho position to the same bridgehead atom (an atom at the junction of two fused rings) between two fused rings in R⁶, with the —N═ ring member in one of the fused rings and the —NH— ring member in the other fused ring. For example, R⁶ is a 9 to 11-membered fused bicyclic heteroaryl of the formula:

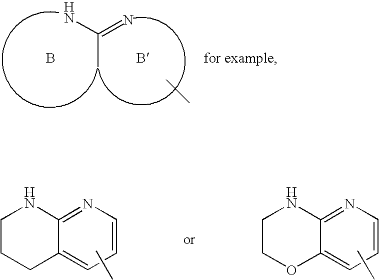

wherein B and B′ are, for example, both 5 or 6-membered monocyclic heteroaryl groups containing nitrogen and optionally one or more (for example 1 or 2) additional heteroatoms selected from O, S and N, or one of B and B is a 5 or 6-membered monocyclic heteroaryl group and the other is a 4 to 7 membered heterocyclic group, wherein B and B′ contain nitrogen and optionally one or more (for example 1 or 2) additional heteroatoms selected from O, S and N.

It is also to be understood that when R⁶ is substituted in an ortho position to the —N═ atom in R⁶ by at least one —NHR^{31a} group, the NHR^{31a} group is located on an adjacent ring atom to the —N═ group, for example R⁶ is: 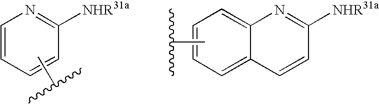
wherein [symbol] indicates the point of attachment of R⁶ to Z.

Similarly, when is stated that Z is NH and R⁶ is attached to Z by a ring carbon atom in an ortho position to the —N═ atom in R⁶, the carbon atom linked to Z is adjacent to the —N═ group in R⁶. For example, R⁶-Z- is a group of the formula:

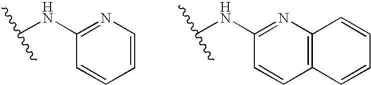

wherein [symbol] indicates the point of attachment of R⁶-Z- to —Y— in formula I. In these embodiments when Z is stated to be NH, it is not substituted by R²⁶.

Unless stated otherwise R⁶ may optionally contain 1 or more additional heteroatoms selected from O, S and N, including one or more —NH— groups (which may be substituted by R³⁵) and R⁶ is optionally substituted on carbon by R³¹.

Where it is stated herein that the group Z-R⁶ has a pKa which is greater than or equal to about 6, the group Z-R⁶, together with any R³¹, R^{31a} or R³⁵ substituents has a pKa greater than or equal to about 6, for example a pKa in the range of from about 6 to about 12, for example from 6 to 9. The pKa of the group Z-R⁶ may be determined using routine methods. For example pKa may be measured using multiwavelength spectrophotometry to determine acid dissociation constants as described in:
Multiwavelength spectrophotometric determination of acid dissociation constants of ionizable drugs; Allen, R. I.; Box, K. J.; Corner, J. E. A.; Peake, C.; Tam, K. Y. Sirius Analytical Instruments Ltd, East Sussex, UK. Journal of Pharmaceutical and Biomedical Analysis (1998), 17(4,5); and
Multiwavelength spectrophotometric determination of acid dissociation constants Part IV. Water-insoluble pyridine derivatives. Tam, K. Y.; Hadley, M.; Patterson, W. Sirius Analytical Instruments Ltd., East Sussex, UK. Talanta (1999), 49(3), 539-546.

In particular the pKa may be determined using multiwavelength spectrophotometry in a Sirius GlpKa instrument equipped with the D-PAS accessory as follows. A stock solution of the compound in DMSO is prepared (1.5 mg/ml). 50 μl of this solution are added to 250 μl of phosphate buffer (2 mg/ml) and diluted in 20 ml of ionic strength adjusted water (KCl 0.15 M). The pH is then automatically adjusted to pH 2.5 with 0.5 M hydrochloric acid and the titration performed by adding 0.5 M potassium hydroxide. For each titration point the UV spectrum is recorded. The pKa values are calculated from the UV modifications with the Sirius pKaUV software.

There are also well known references that list the pKa values of various groups, for example:
Handbook of Biochemistry and Molecular Biology, Physical and Chemical Data, Vol. 1. 3rd Ed. Fasman, G. D.; Editor. (1976), 552 pp. Publisher: (CRC Press, Cleveland, Ohio).
Comprehensive Heterocyclic Chemistry II: An Extended and Updated Review of the Literature, 1982-1995, 2nd Edition. Katritzky, Alan R.; Rees, Charles W.; Scriven, Eric F. V; (1996), 9500 pp. Publisher: (Pergamon, Oxford, UK); and
pKa Prediction for Organic Acids and Bases. Perrin, D. D.; Dempsey, Boyd; Serjeant, E. P. UK. (1981), 150 pp. Publisher: (Chapman and Hall, London).

Various software packages are also available for the estimation of pKa, for example ACD Labs.

When it is stated herein that R⁶ is linked to Z by a carbon atom in an aromatic ring in R⁶, this refers to a carbon atom in an aromatic portion of the heteroaryl ring system represented by R⁶. For example when R⁶ is a bicyclic heteroaryl ring and one ring is aromatic and the other ring is non-aromatic or partially aromatic, then R⁶ is linked to Z by a carbon atom in the aromatic ring of R⁶. For example R⁶ may be:

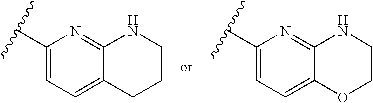

It is to be understood that when n is 0, the group 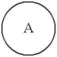
does not carry an R⁵ substituent.

As will be understood references to the group 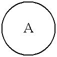
being phenyl or pyridinyl are intended to refer to divalent phenylene and pyridin-di-yl moieties such as:

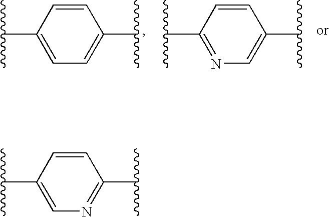

Similarly references to the group 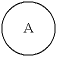
being thiophenyl is intended to refer to divalent thienyl moieties such as:

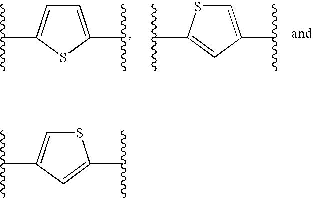

The various functional groups and substituents making up the compounds of the formula I are typically chosen such that the molecular weight of the compound of the formula I does not exceed 1000. More usually, the molecular weight of the compound will be less than 750, for example less than 700, or less than 650, or less than 600, or less than 550. More preferably, the molecular weight is less than 525 and, for example, is 500 or less.

Compounds that have the same molecular formula but differ in the nature or sequence of bonding of their atoms or the arrangement of their atoms in space are termed “isomers”. Isomers that differ in the arrangement of their atoms in space are termed “stereoisomers”. Stereoisomers that are not mirror images of one another are termed “diastereomers” and those that are non-superimposable mirror images of each other are termed “enantiomers”. When a compound has an asymmetric center, for example, it is bonded to four different groups, a pair of enantiomers is possible. An enantiomer can be characterized by the absolute configuration of its asymmetric center and is described by the R- and S-sequencing rules of Cahn and Prelog, or by the manner in which the molecule rotates the plane of polarized light and designated as dextrorotatory or levorotatory (i.e., as (+) or (−)-isomers respectively). A chiral compound can exist as either individual enantiomer or as a mixture thereof. A mixture containing equal proportions of the enantiomers is called a “racemic mixture”.

The compounds of this invention may possess one or more asymmetric centers; such compounds can therefore be produced as individual (R)- or (S)-stereoisomers or as mixtures thereof. Unless indicated otherwise, the description or naming of a particular compound in the specification and claims is intended to include both individual enantiomers and mixtures, racemic or otherwise, thereof. The methods for the determination of stereochemistry and the separation of stereoisomers are well-known in the art (see discussion in Chapter 4 of “Advanced Organic Chemistry”, 4th edition J. March, John Wiley and Sons, New York, 2001), for example by synthesis from optically active starting materials or by resolution of a racemic form. Some of the compounds of the invention may have geometric isomeric centres (E- and Z-isomers). It is to be understood that the present invention encompasses all optical, diastereoisomers and geometric isomers and mixtures thereof that possess a5b1 antagonistic activity. The present invention also encompasses all tautomeric forms of the compounds of formula I that possess a5b1 antagonistic activity.

It is also to be understood that certain compounds of the formula I may exist in solvated as well as unsolvated forms such as, for example, hydrated forms. It is to be understood that the invention encompasses all such solvated forms that possess a5b 1 antagonistic activity.

It is also to be understood that certain compounds of the formula I may exhibit polymorphism, and that the invention encompasses all such forms that possess a5b1 antagonistic activity.

Compounds of the formula I may exist in a number of different tautomeric forms and references to compounds of the formula I include all such forms. For the avoidance of doubt, where a compound can exist in one of several tautomeric forms and only one is specifically described or shown, all others are nevertheless embraced by formula I. For example, when R⁶ is benzimidazolyl both tautomers of the benzimidazolyl group are possible for R⁶:

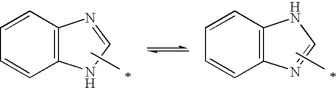

Other examples of tautomeric forms include keto-, enol-, and enolate-forms, as in, for example, the following tautomeric pairs: keto/enol (illustrated below), imine/enamine, amide/imino alcohol, amidine/amidine, nitroso/oxime, thioketone/enethiol, and nitro/aci-nitro.

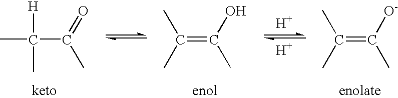

Compounds of the formula I containing an amine function may also form N-oxides. A reference herein to a compound of the formula I that contains an amine function also includes the N-oxide. Where a compound contains several amine functions, one or more than one nitrogen atom may be oxidised to form an N-oxide. Particular examples of N-oxides are the N-oxides of a tertiary amine or a nitrogen atom of a nitrogen-containing heterocycle. N-Oxides can be formed by treatment of the corresponding amine with an oxidizing agent such as hydrogen peroxide or a per-acid (e.g. a peroxycarboxylic acid), see for example Advanced Organic Chemistry, by Jerry March, 4^{th} Edition, Wiley Interscience, pages. More particularly, N-oxides can be made by the procedure of L. W. Deady (Syn. Comm. 1977, 7, 509-514) in which the amine compound is reacted with m-chloroperoxybenzoic acid (MCPBA), for example, in an inert solvent such as dichloromethane.

A “pharmaceutically acceptable excipient” means an excipient that is useful in preparing a pharmaceutical composition that is generally safe, non-toxic and neither biologically nor otherwise undesirable, and includes an excipient that is acceptable for veterinary use as well as human pharmaceutical use. A “pharmaceutically acceptable excipient” as used in the specification and claims includes both one and more than one such excipient.

A “pharmaceutically acceptable counter ion” means an ion having a charge opposite to that of the substance with which it is associated and that is pharmaceutically acceptable. Representative examples include, but are not limited to, chloride, bromide, iodide, methanesulfonate, p-tolylsulfonate, trifluoroacetate, acetate, and the like.

A “pharmaceutically acceptable salt” of a compound means a salt that is pharmaceutically acceptable and that possesses the desired pharmacological activity of the parent compound. Such pharmaceutically-acceptable salts of a compound of the formula I is, for example, an acid-addition salt of a compound of the formula I, for example an acid-addition salt with an inorganic or organic acid such as hydrochloric, hydrobromic, sulfuric, trifluoroacetic, citric or maleic acid; or, for example, a salt of a compound of the formula I which is sufficiently acidic, for example an alkali or alkaline earth metal salt such as a calcium or magnesium salt, or an ammonium salt, or a salt with an organic base such as methylamine, dimethylamine, trimethylamine, piperidine, morpholine or tris-(2-hydroxyethyl)amine.

In particular, compounds of the invention may form internal salts or zwitterions, and these form a particular aspect of the invention. Thus, for example, whilst the compounds are drawn and referred to in say the hydroxyl form, they may exist also in internal salt (zwitterionic) form, such as a zwitterion with a basic group in R⁶ as depicted below:

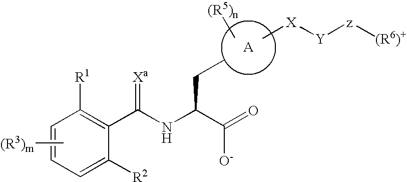

The representation of formula I and the examples of the present invention covers both hydroxyl and zwitterionic forms and mixtures thereof in all proportions.

“Leaving group” has the meaning conventionally associated with it in synthetic organic chemistry i.e., an atom or group capable of being displaced by a nucleophile and includes halogen (such as chloro, bromo, iodo), alkanesulfonyloxy (such as mesyloxy or trifluorosulfonyloxy) or arenesulfonyloxy (such as tosyloxy), and the like. Leaving Groups are well known in the art and are catalogued in “Protective Groups in Organic Synthesis 3^{rd} Ed.”, edited by Theodora Green and Peter Wuts (John Wiley, 1999).

The compounds of formula I may be administered in the form of a pro-drug which is broken down in the human or animal body to give a compound of the formula I. A “Pro-drug” is any compound which releases an active parent drug according to formula I in vivo when such prodrug is administered to a mammalian subject. Prodrugs of a compound of formula I are prepared by modifying functional groups present in the compound of formula I in such a way that the modifications may be cleaved in vivo to release the parent compound. Examples of prodrugs include, but are not limited to esters (e.g., acetate, formate, and benzoate derivatives), carbamates (e.g., N,N-dimethylaminocarbonyl) of hydroxy or carboxy functional groups in compounds of formula I, and the like.

Various forms of pro-drugs are known in the art. For examples of such pro-drug derivatives, see:
1. Design of Prodrugs, edited by H. Bundgaard, (Elsevier, 1985) and Methods in Enzymology, Vol. 42, p. 309-396, edited by K. Widder, et al. (Academic Press, 1985);
2. A Textbook of Drug Design and Development, edited by Krogsgaard-Larsen and H. Bundgaard, Chapter 5 “Design and Application of Prodrugs”, by H. Bundgaard p. 113-191 (1991);
3. H. Bundgaard, Advanced Drug Delivery Reviews, 8, 1-38 (1992);
4. H. Bundgaard, et al., Journal of Pharmaceutical Sciences, 77, 285 (1988);
5. N. Kakeya, et al., Chem Pharm Bull, 32, 692 (1984);
6. K. Beaumont et. al., Current Drug Metabolism, 4, 461 (2003).

An in-vivo hydrolysable ester of a compound of the Formula (I containing a carboxy or a hydroxy group is, for example, a pharmaceutically-acceptable ester which is hydrolysed in the human or animal body to produce the parent acid or alcohol. Suitable pharmaceutically-acceptable esters for carboxy include (1-6C)alkoxymethyl esters for example methoxymethyl, (1-6C)alkanoyloxymethyl esters for example pivaloyloxymethyl, phthalidyl esters, (3-8C)cycloalkoxycarbonyloxyC₁₋₆alkyl esters for example 1-cyclohexylcarbonyloxyethyl; 1,3-dioxolen-2-onylmethyl esters, for example 5-methyl-1,3-dioxolen-2-onylmethyl; and (1-6C)alkoxycarbonyloxyethyl esters.

An in-vivo hydrolysable ester of a compound of the formula I containing a hydroxy group includes inorganic esters such as phosphate esters (including phosphoramidic cyclic esters) and α-acyloxyalkyl ethers and related compounds which as a result of the in-vivo hydrolysis of the ester breakdown to give the parent hydroxy group/s. Examples of α-acyloxyalkyl ethers include acetoxymethoxy and 2,2-dimethylpropionyloxy-methoxy. A selection of in-vivo hydrolysable ester forming groups for hydroxy include alkanoyl, benzoyl, phenylacetyl and substituted benzoyl and phenylacetyl, alkoxycarbonyl (to give alkyl carbonate esters), dialkylcarbamoyl and N-(dialkylaminoethyl)-N-alkylcarbamoyl (to give carbamates), dialkylaminoacetyl and carboxyacetyl.

When R⁴ is other than H, the compounds of formula I may behave as pro-drugs with the R⁴ group being hydrolysed in-vivo to give the free carboxy group. As will be understood, such compounds and other prodrugs of formula I may exhibit low activity in the in-vitro assays described herein compared to the free carboxy compound. However, such compounds are expected to show activity under conditions that result in the hydrolysis of the R⁴ group to give the free carboxy group.

“Treating” or “treatment” of a disease includes:

1. preventing the disease, i.e. causing the clinical symptoms of the disease not to develop in a mammal that may be exposed to or predisposed to the disease but does not yet experience or display symptoms of the disease;
2. inhibiting the disease, i.e., arresting or reducing the development of the disease or its clinical symptoms; or
3. relieving the disease, i.e., causing regression of the disease or its clinical symptoms.
A “therapeutically effective amount” means the amount of a compound that, when administered to a mammal for treating a disease, is sufficient to effect such treatment for the disease. The “therapeutically effective amount” will vary depending on the compound, the disease and its severity and the age, weight, etc., of the mammal to be treated.

The phrase “compound of the invention” means those compounds which are disclosed herein, both generically and specifically.

Particular novel compounds of the invention include, for example, compounds of the formula I, or pharmaceutically acceptable salts and pro-drugs thereof, wherein, unless otherwise stated, each of m, n, R¹, R², R³, R⁴, R⁵, R¹, X, Y and Z has any of the meanings defined hereinbefore or in paragraphs (1) to (124) hereinafter:—

(1) R¹ is selected from chloro, (1-3C)alkyl and trifluoromethyl.

(2) R¹ is selected from chloro and (1-3C)alkyl.

(3) R¹ is selected from chloro, bromo, methyl, ethyl, cyclopropyl, cyclobutyl and trifluoromethyl.

(4) R¹ is selected from bromo, chloro, methyl, ethyl and cyclopropyl.

(5) R¹ is selected from chloro, methyl and ethyl.

(6) R¹ is selected from methyl, ethyl or cyclopropyl.

(7) R¹ is methyl or ethyl.

(8) R¹ is methyl or trifluoromethyl.

(9) R¹ is selected from bromo or chloro.

(10) R¹ is chloro or methyl.

(11) R¹ is chloro.

(12) R¹ is methyl.

(13) R² is selected from hydrogen, halo, trifluoromethyl, (1-6C)alkyl, and (1-6C)alkoxy.

(14) R² is selected from hydrogen, halo and (1-4C)alkyl, or R² and an adjacent R³ group optionally form a (1-3C)alkylenedioxy group (for example methylenedioxy or ethylenedioxy).

(15) R² is selected from hydrogen, fluoro, chloro, bromo, methyl, ethyl and trifluoromethyl or R² and an adjacent R³ group optionally form a methylenedioxy group.

(16) R² is selected from hydrogen, fluoro, chloro, bromo and methyl.

(17) R² is selected from hydrogen and chloro.

(18) R² is selected from chloro, fluoro and methyl.

(19) R² is hydrogen.

(20) R² is fluoro or chloro.

(21) R² is chloro.

(22) R¹ is selected from chloro, bromo, methyl, ethyl, trifluoromethyl and cyclopropyl, and R² is selected from hydrogen, fluoro, chloro, bromo, methyl, ethyl, trifluoromethyl cyclopropyl,

or R² and an adjacent R³ group optionally form a methylenedioxy group.

(23) R¹ is chloro or methyl and R² is selected from hydrogen, fluoro, chloro and methyl, for example R¹ and R² are both methyl, or R¹ is chloro and R² is fluoro;

(24) R¹ and R² are both chloro.

(25) m is 0, 1 or 2 and each R³, which may be the same or different, is selected from halo, trifluoromethyl, cyano, hydroxy, mercapto, amino, carbamoyl, sulfamoyl, (1-6C)alkyl, (1-6C)alkoxy, (1-6C)alkylthio, (1-6C)alkylsulfinyl, (1-6C)alkylsulfonyl, (1-6C)alkylamino, di-[(1-6C)alkyl]amino, N-(1-6C)alkylcarbamoyl, N,N-di-[(1-6C)alkyl]carbamoyl, (2-6C)alkanoylamino, N-(1-6C)alkyl-(2-6C)alkanoylamino, N-(1-6C)alkylsulfamoyl, N,N-di-[(1-6C)alkyl]sulfamoyl, (1-6C)alkanesulfonylamino and N-(1-6C)alkyl-(1-6C)alkanesulfonylamino,

or from a group of the formula:
Q¹-X¹—
wherein X¹ is a direct bond or is selected from O, S, SO, SO₂ or N(R⁷), wherein R⁷ is hydrogen or (1-6C)alkyl, and Q¹ is (3-7C)cycloalkyl, (3-7C)cycloalkyl-(1-6C)alkyl, phenyl, phenyl-(1-6C)alkyl, heteroaryl, heteroaryl-(1-6C)alkyl, heterocyclyl, heterocyclyl-(1-6C)alkyl,

wherein any heteroaryl in R³ is a 5 or 6 membered monocyclic heteroaryl containing 1, 2 or 3 heteroatoms selected from O, S and N,

and wherein any heterocyclyl in R³ is selected from a 4 to 7 membered monocyclic heterocyclyl ring containing 1, 2 or 3 heteroatoms selected from O, S and N,

and wherein any R³ optionally bears on carbon one or more substituents selected from halo, cyano, nitro, hydroxy, amino, carbamoyl, sulfamoyl, (1-6C)alkyl, (2-6C)alkenyl, (2-6C)alkynyl, (1-6C)alkoxy, (1-6C)alkylsulfonyl, (1-6C)alkylamino, di-[(1-6C)alkyl]amino, N-(1-6C)alkylcarbamoyl, N,N-di-[(1-6C)alkyl]carbamoyl, (2-6C)alkanoylamino, N-(1-6C)alkyl-(2-6C)alkanoylamino, N-(1-6C)alkylsulfamoyl, N,N-di-[(1-6C)alkyl]sulfamoyl, (1-6C)alkanesulfonylamino and N-(1-6C)alkyl-(1-6C)alkanesulfonylamino,

and wherein if any heteroaryl or heterocyclyl group within any R³ contains an —NH— moiety, the nitrogen of said moiety optionally bears a group selected from R⁹, wherein R⁹ is selected from carbamoyl, sulfamoyl, (1-6C)alkyl, (2-8C)alkenyl, (2-8C)alkynyl, (1-6C)alkylsulfonyl, N-(1-6C)alkylcarbamoyl, N,N-di-[(1-6C)alkyl]carbamoyl, (2-6C)alkanoyl, N-(1-6C)alkylsulfamoyl and N,N-di-[(1-6C)alkyl]sulfamoyl,

or from a group of the formula:
Q¹-X¹—

wherein X¹ is a direct bond or is selected from C(O), SO₂, C(O)N(R⁷) and SO₂N(R⁷), wherein R⁷ is hydrogen or (1-6C)alkyl, and Q¹ is (3-7C)cycloalkyl or (3-7C)cycloalkyl-(1-6C)alkyl,

and wherein any heterocyclyl group within R² and any R³ optionally bears 1 or 2 oxo or thioxo substituents;

or two R³ substituents optionally form a (1-3C)alkylenedioxy group,

or R² and an adjacent R³ group optionally form a (1-3C)alkylenedioxy group.

(26) m is 0, 1 or 2 and each R³, which may be the same or different, is selected from halo, trifluoromethyl, cyano, nitro, hydroxy, mercapto, amino, carboxy, carbamoyl, sulfamoyl, (1-6C)alkyl, (2-8C)alkenyl, (2-8C)alkynyl, (1-6C)alkoxy, (2-6C)alkenyloxy, (2-6C)alkynyloxy, (1-6C)alkylthio, (1-6C)alkylsulfinyl, (1-6C)alkylsulfonyl, (1-6C)alkylamino, di-[(1-6C)alkyl]amino, (1-6C)alkoxycarbonyl, N-(1-6C)alkylcarbamoyl, N,N-di-[(1-6C)alkyl]carbamoyl, (2-6C)alkanoyl, (2-6C)alkanoyloxy, (2-6C)alkanoylamino, N-(1-6C)alkyl-(2-6C)alkanoylamino, (3-6C)alkenoylamino, N-(1-6C)alkyl-(3-6C)alkenoylamino, (3-6C)alkynoylamino, N-(1-6C)alkyl-(3-6C)alkynoylamino, N-(1-6C)alkylsulfamoyl, N,N-di-[(1-6C)alkyl]sulfamoyl, (1-6C)alkanesulfonylamino and N-(1-6C)alkyl-(1-6C)alkanesulfonylamino,

or from a group of the formula:
Q¹-X¹—
wherein X¹ is a direct bond or is selected from O, S, SO, SO₂, N(R⁷), C(O), CH(OR⁷), C(O)N(R⁷), N(R⁷)C(O), SO₂N(R⁷), N(R⁷)SO₂, OC(R⁷)₂, SC(R⁷)₂ and N(R⁷)C(R⁷)₂, wherein R⁷ is hydrogen or (1-6C)alkyl, and Q¹ is (3-7C)cycloalkyl or (3-7C)cycloalkyl-(1-6C)alkyl,

and wherein any R³ optionally bears on carbon one or more substituents selected from halo, trifluoromethyl, cyano, nitro, hydroxy, amino, carboxy, carbamoyl, sulfamoyl, (1-6C)alkyl, (2-8C)alkenyl, (2-8C)alkynyl, (1-6C)alkoxy, (1-6C)alkylsulfonyl, (1-6C)alkylamino, di-[(1-6C)alkyl]amino, (1-6C)alkoxycarbonyl, N-(1-6C)alkylcarbamoyl, N,N-di-[(1-6C)alkyl]carbamoyl, (2-6C)alkanoyl, (2-6C)alkanoyloxy, (2-6C)alkanoylamino, N-(1-6C)alkyl-(2-6C)alkanoylamino, N-(1-6C)alkylsulfamoyl, N,N-di-[(1-6C)alkyl]sulfamoyl, (1-6C)alkanesulfonylamino and N-(1-6C)alkyl-(1-6C)alkanesulfonylamino,

or two R³ substituents optionally form a (1-3C)alkylenedioxy group.

(27) m is 0, 1 or 2 and each R³, which may be the same or different, is selected from halo, trifluoromethyl, hydroxy, amino, carbamoyl, sulfamoyl, (1-6C)alkyl, (1-6C)alkoxy, (1-6C)alkylthio, (1-6C)alkylsulfinyl, (1-6C)alkylsulfonyl, (1-6C)alkylamino, di-[(1-6C)alkyl]amino, N-(1-6C)alkylcarbamoyl, N,N-di-[(1-6C)alkyl]carbamoyl, (2-6C)alkanoylamino, N-(1-6C)alkyl-(2-6C)alkanoylamino, N-(1-6C)alkylsulfamoyl, N,N-di-[(1-6C)alkyl]sulfamoyl, (1-6C)alkanesulfonylamino, N-(1-6C)alkyl-(1-6C)alkanesulfonylamino, a heterocyclyl selected from azetidinyl, pyrrolidinyl, piperidinyl, morpholinyl and piperazinyl, a heteroaryl selected from pyrrolyl, furanyl, 1,3-oxazolyl, 1,3-thiazolyl, isoxazolyl, isothiazolyl, pyrazolyl, imidazolyl, pyridyl, pyrimidinyl and pyrazinyl or phenyl,

and wherein any phenyl, heteroaryl or heterocyclyl in R³ optionally bears on carbon one or more substituents selected from halo, cyano, nitro, hydroxy, amino, (1-6C)alkyl, (2-6C)alkenyl, (2-6C)alkynyl, (1-6C)alkoxy, (1-6C)alkylsulfonyl, (1-6C)alkylamino and di-[(1-6C)alkyl]amino,

and wherein if any heteroaryl or heterocyclyl group in an R³ contains an —NH-moiety, the nitrogen of said moiety optionally bears a group selected from (1-6C)alkyl and (2-6C)alkanoyl,

and wherein any heterocyclyl group within any R³ optionally bears 1 oxo substituent,

or two R³ substituents optionally form a (1-3C)alkylenedioxy group,

or R² and an adjacent R³ group optionally form a (1-3C)alkylenedioxy group.

(28) m is 0, 1 or 2 and each R³, which may be the same or different, is selected from halo, trifluoromethyl, hydroxy, amino, (1-6C)alkyl, (2-8C)alkenyl, (2-8C)alkynyl, (1-6C)alkoxy, (1-6C)alkylsulfonyl, (1-6C)alkylamino, di-[(1-6C)alkyl]amino, (1-6C)alkoxycarbonyl, (2-6C)alkanoyl, (2-6C)alkanoyloxy, amino-(1-6C)alkyl, (1-6C)alkylamino-(1-6C)alkyl and di-[(1-6C)alkyl]amino-(1-6C)alkyl,

or two R³ substituents optionally form a (1-3C)alkylenedioxy group.

(29) m is 0, 1 or 2 and each R³, which may be the same or different, is selected from fluoro, chloro, bromo trifluoromethyl, hydroxy, carbamoyl, sulfamoyl, (1-4C)alkyl, (1-4C)alkoxy, (1-4C)alkylsulfonyl, N-(1-4C)alkylcarbamoyl, N,N-di-[(1-4C)alkyl]carbamoyl, N-(1-4C)alkylsulfamoyl, N,N-di-[(1-4C)alkyl]sulfamoyl, (2-4C)alkanoylamino, a heterocyclyl selected from pyrrolidinyl, piperidinyl and morpholinyl or R³ is pyrazolyl,

and wherein any heterocyclyl or pyrazolyl in R³ optionally bears on carbon one or more substituents selected from halo, hydroxy, amino, (1-4C)alkyl, (1-4C)alkoxy, (1-4C)alkylsulfonyl, (1-4C)alkylamino and di-[(1-4C)alkyl]amino,

and wherein if any heteroaryl or pyrazolyl in R³ contains an —NH— moiety, the nitrogen of said moiety optionally bears a group selected from (1-4C)alkyl and (2-4C)alkanoyl,

and wherein any heterocyclyl group within any R³ optionally bears 1 oxo substituent;

or two R³ substituents form a (1-3C)alkylenedioxy group,

or R² and an adjacent R³ group optionally form a (1-3C)alkylenedioxy group.

(30) m is 0, 1 or 2 and each R³, which may be the same or different, is selected from halo and (1-4C)alkyl, or two R³ substituents optionally form a (1-3C)alkylenedioxy group.

(31) m is 0, 1 or 2 and each R³, which may be the same or different, is selected from fluoro, chloro, bromo and (1-3C)alkyl.

(32) m is 0 or 1 and R³ is selected from fluoro, chloro and methyl.

(33) m is 0.

(34) R¹ is selected from chloro, bromo, methyl, ethyl, cyclopropyl and trifluoromethyl;

R² is selected from hydrogen, chloro, bromo, methyl, ethyl, cyclopropyl and trifluoromethyl,

or R² and an adjacent R³ group optionally form a (1-3C)alkylenedioxy group, and m and R³ are as defined in any one of (25) to (33) above.
(35) The group: 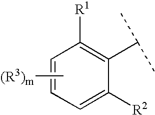
in formula I is selected from 2-acetylamino-6-chlorophenyl, 4-acetylamino-2-methylphenyl, 2-bromo-6-chlorophenyl, 2-bromo-4,5-difluorophenyl, 2-bromo-4-fluorophenyl, 2-bromo-5-fluorophenyl, 2-chlorophenyl, 2-chloro-3,6-difluorophenyl, 2-chloro-3,4-dimethoxyphenyl, 2-chloro-4,5-dimethoxyphenyl, 2-chloro-6-fluorophenyl, 2-chloro-4-fluorophenyl, 2-chloro-3-fluorophenyl, 2-chloro-5-fluorophenyl, 2-chloro-4-fluoro-5-sulfamoylphenyl, 3-chloro-2-fluoro-6-trifluoromethylphenyl, 6-chloro-2-fluoro-3-methylphenyl, 2-chloro-6-fluoro-3-methylphenyl, 6-chloro-2-fluoro-3-methylphenyl, 2-chloro-4-methoxyphenyl, 2-chloro-5-methoxyphenyl, 2-chloro-4-methylsulfonylphenyl, 2-chloro-5-methylphenyl, 2-chloro-3-methylphenyl, 2-chloro-6-methylphenyl, 4-chloro-2-methylphenyl, 5-chloro-2-methylphenyl, 3-chloro-2-methylphenyl, 2-chloro-4-morpholin-4-ylphenyl, 2-chloro-4-pyrrolidin-1-ylphenyl, 2-chloro-4-(1H-pyrazol-1-yl)phenyl, 2-chloro-3,6-difluorophenyl, 2-chloro-4,5-difluorophenyl, 2-chloro-5-trifluoromethylphenyl, 5-chloro-2-trifluoromethylphenyl, 2-chloro-3-trifluoromethylphenyl, 2-cyclopropyl-4-fluorophenyl, 3,6-dichloro-2-methoxyphenyl, 2,4-dichlorophenyl, 2,3-dichlorophenyl, 2,6-dichlorophenyl, 4,5-difluoro-2-methylphenyl, 2,3-dimethylphenyl, 2,4-dimethylphenyl, 2,6-dimethylphenyl, 2,5-dimethylphenyl, 2,4-dichloro-5-fluorophenyl, 2,6-dichloro-3-methylphenyl, 2-ethylphenyl, 2-ethyl-4-fluorophenyl, 2-ethyl-5-fluorophenyl, 3-fluoro-2-methylphenyl, 5-fluoro-2-methylphenyl, 4-fluoro-2-methylphenyl, 2-fluoro-6-methylphenyl, 2-fluoro-6-trifluoromethylphenyl, 3-fluoro-2-trifluoromethylphenyl, 5-fluoro-2-trifluoromethylphenyl, 4-fluoro-2-trifluoromethylphenyl, 3-hydroxy-2-methylphenyl, 2-methylphenyl, 2-methyl-3-trifluoromethylphenyl, 2-methyl-5-trifluoromethylphenyl, 4-methoxy-2-methylphenyl, 3-methoxy-2-methylphenyl, 4-methoxy-2-trifluoromethylphenyl, 5-chloro-1,3-benzodioxol-4-yl 2-trifluoromethylphenyl, 2,4,6-trimethylphenyl and 2,4,5-trimethylphenyl.
(35a) The group: 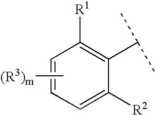
in formula I is selected from 2-chloro-6-methylphenyl, 2,4-dichlorophenyl, 2-chloro-3,6-difluorophenyl, 2-chloro-6-fluorophenyl, 2-chloro-6-fluoro-3-methylphenyl, 6-chloro-2-fluoro-3-methylphenyl, 2-chloro-5-methylphenyl, 3-chloro-2-methylphenyl, 2-methylphenyl and 2,4,6-trimethylphenyl.
(35b) The group: 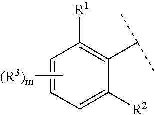
in formula I is selected from 2-bromo-6-chlorophenyl, 2-chlorophenyl, 2-chloro-4-fluorophenyl, 2,6-dichloro-3-methylphenyl, 2,6-dimethylphenyl and 5-chloro-1,3-benzodioxol-4-yl.
(36) The group: 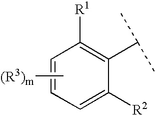
in formula I is selected from 2-chloro-6-methylphenyl, 2,6-dichlorophenyl, 2,4-dichlorophenyl, 2-chlorophenyl, 2,6-dimethylphenyl, 2-chloro-6-fluorophenyl, 2-chloro-4-fluorophenyl, 5-chloro-1,3-benzodioxol-4-yl, 2-chloro-3,6-difluorophenyl, 2-fluoro-6-methylphenyl, 2-chloro-4-methoxyphenyl, 2-ethyl-4-fluorophenyl, 2,4,6-trimethylphenyl, and 6-chloro-2-fluoro-3-methylphenyl.
(36a) The group: 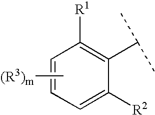
in formula I is selected from 2,6-dichlorophenyl, 2-chlorophenyl, 2-chloro-4-fluorophenyl, 2-chloro-6-fluorophenyl, 2-chloro-6-methylphenyl, 2-fluoro-6-methylphenyl, 2-chloro-4-methoxyphenyl, 2-ethyl-4-fluorophenyl and 2,6-dimethylphenyl.
(37) R⁴ is selected from hydrogen and (1-6C)alkyl, wherein R⁴ is optionally bears on carbon one or more R²¹ substituents selected from halo, hydroxy and (1-4C)alkoxy;
(38) R⁴ is selected from hydrogen and (1-4C)alkyl, wherein R⁴ is optionally bears on carbon a hydroxy substituent, for example R⁴ is selected from hydrogen, methyl, ethyl, propyl, isopropyl, butyl, isobutyl, tert-butyl 2-hydroxyethyl and 3-hydroxybutyl;
(39) R⁴ is selected from monocyclic heterocyclyl or heterocyclyl(1-6C)alkyl, and wherein any heterocyclyl in R⁴ optionally bears 1 or more substituents selected from (1-4C)alkyl and oxo.
(40) R⁴ is selected from hydrogen, methyl, ethyl, hydroxyethyl, iso-propyl, 2-(diethylaminoethyl) or a group of the formula: 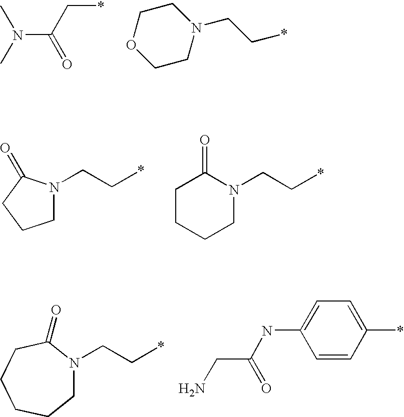
where * indicates the point of attachment to the oxygen atom.
(41) R⁴ is hydrogen;
(42) n is 0, 1, 2, 3 or 4, provided that when ring A is pyridinyl, n is 0, 1, 2 or 3 and that when ring A is thiophenyl, n is 0, 1 or 2;

each R⁵, which may be the same or different, is selected from halo, trifluoromethyl, cyano, isocyano, nitro, mercapto, amino, formyl, carboxy, carbamoyl, sulfamoyl, (1-6C)alkyl, (2-8C)alkenyl, (2-8C)alkynyl, (1-6C)alkoxy, (2-6C)alkenyloxy, (2-6C)alkynyloxy, (1-6C)alkylthio, (1-6C)alkylsulfinyl, (1-6C)alkylsulfonyl, (1-6C)alkylamino, di-[(1-6C)alkyl]amino, (1-6C)alkoxycarbonyl, N-(1-6C)alkylcarbamoyl, N,N-di-[(1-6C)alkyl]carbamoyl, (2-6C)alkanoyl, (2-6C)alkanoylamino, N-(1-6C)alkyl-(2-6C)alkanoylamino, (3-6C)alkenoylamino, N-(1-6C)alkyl-(3-6C)alkenoylamino, (3-6C)alkynoylamino, N-(1-6C)alkyl-(3-6C)alkynoylamino, N-(1-6C)alkylsulfamoyl, N,N-di-[(1-6C)alkyl]sulfamoyl, (1-6C)alkanesulfonylamino and N-(1-6C)alkyl-(1-6C)alkanesulfonylamino,

or from a group of the formula:
Q⁵-X⁷—
wherein X⁷ is a direct bond or is selected from O, S, SO, SO₂, N(R²³), C(O), CH(OR²³), C(O)N(R²³), N(R²³)C(O), SO₂N(R²³), N(R²³)SO₂, OC(R²³)₂, SC(R²³)₂ and N(R²³)C(R²³)₂, wherein R²³ is hydrogen or (1-6C)alkyl, and Q⁵ is (3-7C)cycloalkyl, (3-7C)cycloalkyl-(1-6C)alkyl, (3-7C)cycloalkenyl, (3-7C)cycloalkenyl-(1-6C)alkyl, heterocyclyl or heterocyclyl-(1-6C)alkyl,

and wherein R⁵ optionally bears on carbon one or more R²⁴ groups as hereinbefore defined,

and wherein if any heterocyclyl group within R⁵ contains an —NH— moiety, the nitrogen of said moiety optionally bears a group selected from R²⁵ groups as hereinbefore defined,

and wherein any heterocyclyl group within R⁵ optionally bears 1 or 2 oxo or thioxo substituents;

or two R⁵ substituents optionally form a (1-3C)alkylenedioxy group.

(43) n is 0, 1, 2, 3 or 4, provided that when ring A is pyridinyl, n is 0, 1, 2 or 3 and that when ring A is thiophenyl, n is 0, 1 or 2;

each R⁵, which may be the same or different, is selected from hydroxy and (2-6C)alkanoyloxy,

or from a group of the formula:
Q⁵-X⁷—
wherein X⁷ is a direct bond or is selected from O, S, SO, SO₂, N(R²³), C(O), CH(OR²³), C(O)N(R²³), N(R²³)C(O), SO₂N(R²³), N(R²³)SO₂, OC(R²³)₂, SC(R²³)₂ and N(R²³)C(R²³)₂, wherein R²³ is hydrogen or (1-6C)alkyl, and Q⁵ is aryl, aryl-(1-6C)alkyl, heteroaryl or heteroaryl-(1-6C)alkyl,

and wherein R⁵ optionally bears on carbon one or more R²⁴ groups as hereinbefore defined,

and wherein if any heteroaryl group within R⁵ contains an —NH— moiety, the nitrogen of said moiety optionally bears a group selected from R²⁵ groups as hereinbefore defined.

(44) n is 0, 1, 2 or 3 (provided that when ring A is pyridinyl, n is 0, 1, 2 or 3 and that when ring A is thiophenyl, n is 0, 1 or 2) and each R⁵, which may be the same or different, is selected from halo, trifluoromethyl, cyano, nitro, hydroxy, amino, carboxy, carbamoyl, sulfamoyl, (1-6C)alkyl, (2-8C)alkenyl, (2-8C)alkynyl, (1-6C)alkoxy, (2-6C)alkenyloxy, (2-6C)alkynyloxy, (1-6C)alkylthio, (1-6C)alkylsulfinyl, (1-6C)alkylsulfonyl, (1-6C)alkylamino, di-[(1-6C)alkyl]amino, (1-6C)alkoxycarbonyl, N-(1-6C)alkylcarbamoyl, N,N-di-[(1-6C)alkyl]carbamoyl, (2-6C)alkanoyl, (2-6C)alkanoyloxy, (2-6C)alkanoylamino, N-(1-6C)alkyl-(2-6C)alkanoylamino, N-(1-6C)alkylsulfamoyl, N,N-di-[(1-6C)alkyl]sulfamoyl, (1-6C)alkanesulfonylamino and N-(1-6C)alkyl-(1-6C)alkanesulfonylamino, or from a group of the formula:
Q⁵-X⁷—
wherein X¹ is a direct bond or is selected from O, S, SO, SO₂, N(R²³), C(O), CH(OR²³), C(O)N(R²³), N(R²³)C(O), SO₂N(R²³), N(R²³)SO₂, OC(R²³)₂, SC(R²³)₂ and N(R²³)C(R²³)₂, wherein R²³ is hydrogen or (1-6C)alkyl, and Q⁵ is phenyl-(1-6C)alkyl, (3-6C)cycloalkyl, (3-6C)cycloalkyl-(1-6C)alkyl, heteroaryl-(1-6C)alkyl, heterocyclyl or heterocyclyl-(1-6C)alkyl,

and wherein R⁵ optionally bears on carbon one or more R²⁴ substituents selected from halo, cyano, hydroxy, carboxy, amino, (3-6C)cycloalkyl, (2-6C)alkenyl, (2-6C)alkynyl, (1-6C)alkoxy, (1-6C)alkylamino and di-[(1-6C)alkyl]amino,

and wherein if any heteroaryl or heterocyclyl group within R⁵ contains an —NH-moiety, the nitrogen of said moiety optionally bears an R²⁵ selected from carbamoyl, sulfamoyl, (1-6C)alkyl, (2-8C)alkenyl, (2-8C)alkynyl, (1-6C)alkylsulfonyl, N-(1-6C)alkylcarbamoyl, N,N-di-[(1-6C)alkyl]carbamoyl, (2-6C)alkanoyl, N-(1-6C)alkylsulfamoyl and N,N-di-[(1-6C)alkyl]sulfamoyl, or from a group of the formula:
—X⁶-Q⁴

wherein X⁶ is a direct bond or is selected from C(O), SO₂, C(O)N(R²⁰) and SO₂N(R²), wherein R²⁰ is hydrogen or (1-6C)alkyl, and Q⁴ is (3-7C)cycloalkyl or (3-7C)cycloalkyl-(1-6C)alkyl;

and wherein any heterocyclyl group within R⁵ optionally bears 1 oxo substituent;

or two R⁵ substituents optionally form a (1-3C)alkylenedioxy group.

(45) n is 0, 1 or 2 and each R⁵, which may be the same or different, is selected from halo, trifluoromethyl, cyano, hydroxy, amino, carboxy, (1-6C)alkyl, (2-8C)alkenyl, (2-8C)alkynyl, (1-6C)alkoxy, (1-6C)alkylthio, (1-6C)alkylsulfinyl, (1-6C)alkylsulfonyl, (1-6C)alkylamino, di-[(1-6C)alkyl]amino, (1-6C)alkoxycarbonyl, (2-6C)alkanoyl and (2-6C)alkanoyloxy, or from a group of the formula:
Q⁵-X⁷—
wherein X⁷ is a direct bond or is selected from O, S, SO, SO₂, N(R²³) and C(O), wherein R²³ is hydrogen or (1-6C)alkyl, and Q⁵ is (3-6C)cycloalkyl, (3-6C)cycloalkyl-(1-6C)alkyl, heterocyclyl or heterocyclyl-(1-6C)alkyl, which heterocyclyl is a saturated monocyclic 4 to 7 membered heterocyclyl group containing 1 or 2 heteroatoms selected from O, S and N,

and wherein R⁵ optionally bears on carbon one or more R²⁴ substituents selected from halo, cyano, hydroxy, carboxy, amino, (3-6C)cycloalkyl, (2-6C)alkenyl, (2-6C)alkynyl, (1-6C)alkoxy, (1-6C)alkylamino and di-[(1-6C)alkyl]amino,

and wherein if any heterocyclyl group within R⁵ contains an —NH— moiety, the nitrogen of said moiety optionally bears an R²⁵ selected from carbamoyl, sulfamoyl, (1-6C)alkyl, (2-8C)alkenyl, (2-8C)alkynyl, (1-6C)alkylsulfonyl, N-(1-6C)alkylcarbamoyl, N,N-di-[(1-6C)alkyl]carbamoyl, (2-6C)alkanoyl, N-(1-6C)alkylsulfamoyl and N,N-di-[(1-6C)alkyl]sulfamoyl, or from a group of the formula:
—X⁶-Q⁴

wherein X⁶ is a direct bond or is selected from C(O), SO₂, C(O)N(R²⁰) and SO₂N(R²⁰), wherein R²⁰ is hydrogen or (1-6C)alkyl, and Q⁴ is (3-7C)cycloalkyl or (3-7C)cycloalkyl-(1-6C)alkyl;

and wherein any heterocyclyl group within R⁵ optionally bears 1 oxo substituent.

(46) n is 0, 1 or 2 and each R⁵, which may be the same or different, is selected from halo, trifluoromethyl, cyano, hydroxy, amino, (1-4C)alkyl, (2-4C)alkenyl, (2-4C)alkynyl, (1-4C)alkoxy (1-4C)alkylamino and di-[(1-4C)alkyl]amino, or from a group of the formula:
Q⁵-X⁷—
wherein X⁷ is a direct bond or is selected from O and N(R²³) wherein R²³ is hydrogen or (1-4C)alkyl, and Q⁵ is (3-6C)cycloalkyl, (3-6C)cycloalkyl-(1-4C)alkyl, heterocyclyl or heterocyclyl-(1-4C)alkyl, which heterocyclyl is a saturated monocyclic heterocyclyl group containing 1 or 2 heteroatoms selected from O, S and N,

and wherein R⁵ optionally bears on carbon one or more R²⁴ substituents selected from halo, cyano, hydroxy, amino, (1-4C)alkoxy, (1-4C)alkylamino and di-[(1-4C)alkyl]amino,

and wherein if any heteroaryl or heterocyclyl group within R⁵ contains an —NH-moiety, the nitrogen of said moiety optionally bears an R²⁵ selected from (1-4C)alkyl, (1-4C)alkylsulfonyl and (2-4C)alkanoyl or from a group of the formula:
—X⁶-Q⁴

wherein X⁶ is a direct bond or is selected from C(O) and SO₂, and Q⁴ is (3-7C)cycloalkyl or (3-7C)cycloalkyl-(1-6C)alkyl;

and wherein any heterocyclyl group within R⁵ optionally bears 1 oxo substituent.

(47) n is 0, 1, 2, 3 or 4, provided that when ring A is pyridinyl, n is 0, 1, 2 or 3 and that when ring A is thiophenyl, n is 0, 1 or 2;

each R⁵, which may be the same or different, is selected from halo, trifluoromethyl, cyano, nitro, mercapto, amino, formyl, carboxy, carbamoyl, sulfamoyl, (1-6C)alkyl, (2-8C)alkenyl, (2-8C)alkynyl, (1-6C)alkoxy, (2-6C)alkenyloxy, (2-6C)alkynyloxy, (1-6C)alkylthio, (1-6C)alkylsulfinyl, (1-6C)alkylsulfonyl, (1-6C)alkylamino, di-[(1-6C)alkyl]amino, (1-6C)alkoxycarbonyl, N-(1-6C)alkylcarbamoyl, N,N-di-[(1-6C)alkyl]carbamoyl, (2-6C)alkanoyl, (2-6C)alkanoylamino, N-(1-6C)alkyl-(2-6C)alkanoylamino, (3-6C)alkenoylamino, N-(1-6C)alkyl-(3-6C)alkenoylamino, (3-6C)alkynoylamino, N-(1-6C)alkyl-(3-6C)alkynoylamino, N-(1-6C)alkylsulfamoyl, N,N-di-[(1-6C)alkyl]sulfamoyl, (1-6C)alkanesulfonylamino and N-(1-6C)alkyl-(1-6C)alkanesulfonylamino,

and wherein R⁵ optionally bears on carbon one or more R²⁴ groups selected from halo, cyano, hydroxy, carboxy, amino, (3-6C)cycloalkyl, (2-6C)alkenyl, (2-6C)alkynyl, (1-6C)alkoxy, (1-6C)alkylamino and di-[(1-6C)alkyl]amino,

or two R⁵ substituents optionally form a (1-3C)alkylenedioxy group.

(48) n is 0, 1 or 2 and each R⁵, which may be the same or different, is selected from halo, hydroxy, amino, (1-4C)alkyl, (1-4C)alkoxy (1-4C)alkylamino and di-[(1-4C)alkyl]amino,

and wherein R⁵ optionally bears on carbon one or more R²⁴ substituents selected from halo, hydroxy, amino, (1-4C)alkoxy, (1-4C)alkylamino and di-[(1-4C)alkyl]amino;

(49) n is 0, 1 or 2 and each R⁵, which may be the same or different, is selected from halo, amino, (1-4C)alkyl, (1-4C)alkoxy (1-4C)alkylamino and di-[(1-4C)alkyl]amino,

and wherein R⁵ optionally bears on carbon one or more R²⁴ substituents selected from halo, hydroxy, amino, (1-4C)alkoxy, (1-4C)alkylamino and di-[(1-4C)alkyl]amino.

(50) n is 0;

(51) n is 1 or 2 and R⁵ is as hereinbefore defined, for example, as defined in any of (42) to (49) above.

(52) n is 0 or 1 and R⁵ is as hereinbefore defined, for example, as defined in any of (42) to (49) above.

(53) The group: 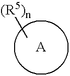
in formula I is selected from phenyl (particularly 1,4-phenylene) and pyridyl, each of which optionally bears n R⁵ substituents wherein n and R⁵ are as hereinbefore defined, for example as defined in any one of (42) to (52) above.
(54) The group: 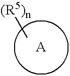
in formula I is phenyl (particularly 1,4-phenylene), which optionally bears n R⁵ substituents wherein n and R⁵ are as hereinbefore defined, for example as defined in any one of (42) to (52) above.
(55) The group: 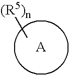
in formula I is pyridyl, which optionally bears n R⁵ substituents wherein n and R⁵ are as hereinbefore defined, for example as defined in any one of (42) to (52) above.
(56) The group: 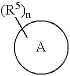
in formula I is thiophenyl, which optionally bears n R⁵ substituents wherein n and R⁵ are as hereinbefore defined, for example as defined in any one of (42) to (52) above.
(57) The group: 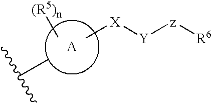
in formula I is: 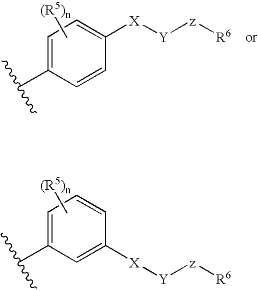
wherein n, R⁵, R⁶, X, Y and Z are as hereinbefore defined.
(58) The group: 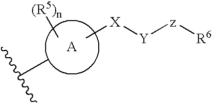
in formula I is: 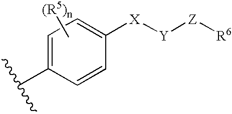
wherein n, R⁵, R⁶, X, Y and Z are as hereinbefore defined. For example the group —X—Y-Z- is (CH₂)₃ or *O(CH₂)₂, wherein * indicates the point of attachment to the phenylene ring.
(59) The group: 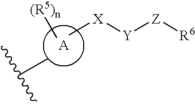
in formula I is: 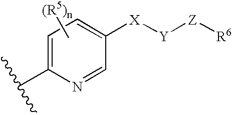
wherein n, R⁵, R⁶, X, Y and Z are as hereinbefore defined. For example the group —X—Y-Z- is (CH₂)₃ or *O(CH₂)₂, wherein * indicates the point of attachment to the pyridinylene ring.
(60) The group: 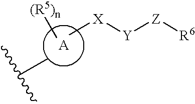
in formula I is: 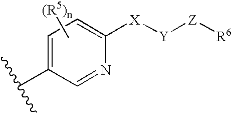
wherein n, R⁵, R⁶, X, Y and Z are as hereinbefore defined. For example the group —X—Y-Z- is (CH₂)₃ or *O(CH₂)₂, wherein * indicates the point of attachment to the pyridinylene ring.
(61) The group: 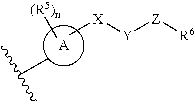
in formula I is: 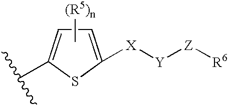
wherein n is 0, 1 or 2 and R⁵, R⁶, Y and Z are as hereinbefore defined; and
X is selected from a direct bond, C(O), CH(OR²⁶), (1-6C)alkylene, CH═CH and C≡C, wherein R²⁶ is as hereinbefore defined (for example X is selected from a direct bond, (1-3C)alkylene, CH═CH and C≡C, more particularly X is a direct bond). For example the group —X—Y-Z- is (CH₂)₃.
(62) X and Z, which may be the same or different, are selected from a direct bond, N(R²⁶), O, (1-6)alkylene, CH═CH and C≡C, wherein R²⁶ is hydrogen, (1-6C)alkyl or (3-7C)cycloalkyl, and wherein any X and Z optionally bears on carbon one or more R²⁸ substituents wherein R²⁸ is as hereinbefore defined;
(63) X is selected from C(O), C(O)N(R²⁶) and N(R²⁶)C(O), wherein R²⁶ is hydrogen, (1-6C)alkyl or (3-7C)cycloalkyl, and wherein any X optionally bears on carbon one or more R²⁸ substituents wherein R²⁸ is as hereinbefore defined.
(64) X and Z are independently selected from C(O), SO₂N(R²⁶), N(R²⁶)SO₂ and CH(OR²⁶), wherein R²⁶ is hydrogen, (1-6C)alkyl or (3-7C)cycloalkyl, and wherein X and Z optionally bears on carbon one or more R²⁸ substituents wherein R²⁸ is as hereinbefore defined.
(65) X is selected from a direct bond, NR⁶, 0 and (1-6C)alkylene, wherein R²⁶ is hydrogen, (1-6C)alkyl or (3-7C)cycloalkyl, and wherein any X optionally bears on carbon one or more R²⁸ substituents wherein R²⁸ is as hereinbefore defined.
(66) X is selected from a direct bond and O.
(67) X is O.
(68) X is a direct bond.
(69) Y is selected from (1-6C)alkylene, (3-7C)cycloalkylene and heterocyclyl,

and wherein adjacent carbon atoms in any (2-6C)alkylene chain within a Y substituent are optionally separated by the insertion into the chain of a group selected from O, N(R²⁷), N(R²⁷)C(O), C(O)N(R²⁷), CH═CH and C≡C wherein R²⁷ is hydrogen, (1-6C)alkyl or (3-7C)cycloalkyl,

and wherein Y optionally bears on carbon one or more R²⁸ substituents, wherein R²⁸ is as hereinbefore defined,

and wherein if any heterocyclyl group within Y contains an —NH— moiety, the nitrogen of said moiety optionally bears a group selected from R²⁹, as hereinbefore defined,

provided that when X is a direct bond or O and Y is a heterocyclyl containing a nitrogen heteroatom, then the group R⁶-Z- is attached to a carbon atom in the heterocyclyl containing the nitrogen heteroatom;

(70) Y is selected from (1-4C)alkylene, (3-6C)cycloalkylene and a monocyclic heterocyclyl which contains 1 or 2 heteroatoms selected from O, S and N, and wherein adjacent carbon atoms in any (2-4C)alkylene chain within a Y substituent are optionally separated by the insertion into the chain of a group selected from 00 N(R²⁷), N(R²⁷)C(O), C(O)N(R²⁷) and C≡C, wherein R²⁷ is hydrogen, (1-4C)alkyl or (3-6C)cycloalkyl,

and wherein Y optionally bears on carbon one or more R²⁸ substituents, wherein R²⁸ is as hereinbefore defined,

and wherein if any heterocyclyl group within Y contains an —NH— moiety, the nitrogen of said moiety optionally bears a group selected from R²⁹, as hereinbefore defined

provided that when X is a direct bond or O and Y is a heterocyclyl containing a nitrogen heteroatom, then the group R⁶-Z- is attached to a carbon atom in the heterocyclyl containing the nitrogen heteroatom;

(71) Y is selected from (1-4C)alkylene, (3-6C)cycloalkylene and a heterocyclyl group selected from azetidinylene, pyrrolidin-di-yl, piperidin-di-yl and piperazin-di-yl, and wherein adjacent carbon atoms in any (2-4C)alkylene chain within a Y substituent are optionally separated by the insertion into the chain of a group selected from O, N(R²⁷) and C≡C wherein R²⁷ is hydrogen or (1-4C)alkyl,

and wherein Y optionally bears on carbon one or more R²⁸ substituents, wherein R²⁸ is as hereinbefore defined,

and wherein if any heterocyclyl group within Y contains an —NH— moiety, the nitrogen of said moiety optionally bears a group selected from R²⁹, as hereinbefore defined,

provided that when X is a direct bond or O and Y is heterocyclyl, then the group R⁶-Z- is attached to a carbon atom in the heterocyclyl containing the nitrogen heteroatom.

(72) Y is a heterocyclyl group selected from azetidin-di-yl, pyrrolidin-di-yl and piperidin-di-yl,

and wherein Y optionally bears on carbon one or more R²⁸ substituents, wherein R²⁸ is as hereinbefore defined.

provided that when X is a direct bond or O, then the group R⁶-Z- is attached to a carbon atom in Y.

(73) X and Z are independently a direct bond or (1-4C)alkyl and Y is a heterocyclyl group selected from azetidin-di-yl, pyrrolidin-di-yl and piperidin-di-yl, which heterocyclyl is attached to the ring A by a ring nitrogen atom in Y,

and wherein Y optionally bears on carbon one or more R²⁸ substituents, wherein R²⁸ is as hereinbefore defined,

and wherein the group R⁶-Z- is attached to a carbon atom in Y.

(74) Y is selected from (1-4C)alkylene, cyclopropylene, cyclobutylene, cyclopentylene, cyclohexylene and a heterocyclyl group selected from azetidin-di-yl, pyrrolidin-di-yl, piperidin-di-yl and piperazin-di-yl, and wherein adjacent carbon atoms in any (2-4C)alkylene chain within a Y substituent are optionally separated by the insertion into the chain of a group selected from O and N(R²⁷), wherein R²⁷ is hydrogen or (1-4C)alkyl,

and wherein Y optionally bears on carbon one or more R²⁸ substituents selected from halo, amino, hydroxy, (1-4C)alkyl, (1-4C)alkoxy, (1-4C)alkylamino and di-[(1-4C)alkyl]amino, and wherein R²⁸ optionally bears on carbon one or more substituents selected from halo, hydroxy, (1-4C)alkoxy and (3-6C)cycloalkyl,

and wherein if any heterocyclyl group within Y contains an —NH— moiety, the nitrogen of said moiety optionally bears an R²⁹ group selected from (1-4C)alkyl, (2-4C)alkanoyl and (1-4C)alkylsulfonyl,

provided that when X is a direct bond or O and Y is a heterocyclyl containing a nitrogen heteroatom, then the group R⁶-Z- is attached to a carbon atom in the heterocyclyl containing the nitrogen heteroatom.

(75) Y is selected from (1-4C)alkylene, (suitably Y is (2-4C)alkylene) and wherein adjacent carbon atoms in any (2-4C)alkylene chain within a Y substituent are optionally separated by the insertion into the chain of a group selected from O, N(R²⁷) and C≡C wherein R²⁷ is hydrogen or (1-4C)alkyl,

and wherein Y optionally bears on carbon one or more R²⁸ substituents selected from halo, amino, (1-4C)alkyl, (1-4C)alkylamino and di-[(1-4C)alkyl]amino, and wherein R²⁸ optionally bears one or more substituents selected from halo, hydroxy, (1-4C)alkoxy, amino, (1-4C)alkylamino, di-[(1-4C)alkyl]amino and (1-6C)cycloalkyl.

(76) Y is selected from (1-4C)alkylene (suitably Y is (2-4C)alkylene), and wherein Y optionally bears on carbon one or more R²⁸ substituents selected from (1-3C)alkyl.

(77) X is a direct bond and Y is a saturated heterocyclyl group which contains at least 1 nitrogen heteroatom and optionally 1 or 2 further heteroatoms selected from O, S and N, which heterocyclyl group is linked to Ring A in formula I by a ring nitrogen in Y, and wherein Y and optionally bears on carbon one or more R²⁸ substituents, wherein R²⁸ is as hereinbefore defined,

and wherein if any heterocyclyl group within Y contains an —NH— moiety, the nitrogen of said moiety optionally bears a group selected from R²⁹, wherein R²⁹ is as hereinbefore defined.

(78) X is a direct bond and Y is a heterocyclyl group selected from azetidin-di-yl, pyrrolidin-di-yl, piperidin-di-yl and piperazin-di-yl, which heterocyclyl group is linked to Ring A in formula I by a ring nitrogen,

and wherein Y optionally bears on carbon one or more R²⁸ substituents selected from halo, amino, (1-4C)alkyl, (1-4C)alkylamino and di-[(1-4C)alkyl]amino, and wherein R²⁸ optionally bears one or more substituents selected from halo, hydroxy, (1-4C)alkoxy and (1-6C)cycloalkyl,

and wherein if any heterocyclyl group within Y contains an —NH— moiety, the nitrogen of said moiety optionally bears an R²⁹ group selected from (1-4C)alkyl, (2-4C)alkanoyl and (1-4C)alkylsulfonyl.

(79) X is a direct bond and Y is a heterocyclyl group selected from azetidin-di-yl, pyrrolidin-di-yl, piperidin-di-yl and piperazin-di-yl, which heterocyclyl group is linked to Ring A in formula I by a ring nitrogen. Suitably in this embodiment Z is selected from a direct bond NR²⁶ (for example NH) and (1-3C)alkylene.

(80) Z is selected from a direct bond, NR²⁶ and (1-4C)alkylene, wherein R²⁶ is hydrogen, (1-4C)alkyl or (3-6C)cycloalkylene, and wherein Z optionally bears on carbon one or more R²⁸ substituents wherein R²⁸ is as hereinbefore defined.

(81) Z is selected from a direct bond and N(R²⁶), wherein R²⁶ is hydrogen, (1-4C)alkyl or (3-6C)cycloalkylene, and wherein Z optionally bears on carbon one or more R²⁸ substituents selected from halo, amino, hydroxy, (1-4C)alkyl, (1-4C)alkoxy, (1-4C)alkylamino and di-[(1-4C)alkyl]amino, and wherein R²⁸ optionally bears on carbon one or more substituents selected from halo, hydroxy, (1-4C)alkoxy and (3-6C)cycloalkyl.

(82) Z is selected from a direct bond and N(R²⁶), wherein R²⁶ is hydrogen or (1-3C)alkyl (for example Z is NH or a direct bond).

(83) X is selected from a direct bond, N(R²⁶), O, C(O)N(R²⁶), N(R²⁶)C(O) and C≡C, wherein R²⁶ is hydrogen, (1-4C)alkyl, (3-6C)cycloalkyl or (3-6C)cycloalkyl-(1-3C)alkyl;

Y is selected from (1-4C)alkylene, and wherein Y optionally bears on carbon one or more R²⁸ substituents selected from (1-3C)alkyl; and

Z is selected from a direct bond and NR²⁶ wherein R²⁶ is hydrogen, (1-4C)alkyl, (3-6C)cycloalkyl or (3-6C)cycloalkyl-(1-3C)alkyl.

(84) X is selected from a direct bond and O;

Y is selected from (1-4C)alkylene (suitably Y is (2-4C)alkylene), and wherein Y optionally bears on carbon one or more R²⁸ substituents selected from (1-3C)alkyl; and

Z is selected from a direct bond and NR²⁶ wherein R²⁶ is hydrogen or (1-3C)alkyl.

(85) Y is (1-6C)alkylene, for example Y is (2-6C)alkylene.

(86) X is oxygen and Y is (1-6C)alkylene, for example X is oxygen and Y is (2-6C)alkylene, suitably X is oxygen and Y is (2-4C)alkylene.

(87) X is oxygen, Y is (1-6C)alkylene and Z is a direct bond.

(88) X, Y and Z have any of the values defined herein, and the group —X—Y-Z- has a chain length of from 3 to 6 atoms, for example 3, 4 or 5 atoms. Particularly when Z is NR²⁶ (for example NH), then the group —X—Y-Z- has a chain length of 5 atoms and when Z is not NR²⁶, then the group —X—Y-Z has a chain length of 3 atoms.

(89) The group —X—Y-Z is selected from —(CH₂)₃—, —(CH₂)₄—, *—O—(CH₂)₂—, *—O—(CH₂)₃—*—(CH₂)₃—N(R²⁶)—, —*—(CH₂)₄—N(R²⁶)—, *—O—(CH₂)₂—N(R²⁶)— and *—O—(CH₂)₃—N(R²⁶)— (particularly the group —X—Y-Z is selected from —(CH₂)₃—, *—O—(CH₂)₂—, *—(CH₂)₄—N(R²⁶)— and *—O—(CH₂)₃—N(R²⁶)—);

wherein * represents the point of attachment to ring A in formula I, and R²⁶ is H or (1-3C)alkyl (suitably R²⁶ is H);

and wherein the group X-Z-Y optionally bears on carbon one or more substituent selected from hydroxy, (1-3C)alkyl, (1-3C)alkoxy, hydroxy-(1-3C)alkyl, (1-3C)alkoxy-(1-3C)alkyl, hydroxy-(2-3C)alkoxy and (1-3C)alkoxy(2-3C)alkoxy (for example X—Y-Z optionally bears on carbon 1 or 2 (1-3C)alkyl substituents).
(90) The group —X—Y-Z is selected from —(CH₂)—, —(CH₂)₂—, *—O—(CH₂)—, *—O—(CH₂)₄—*—(CH₂)₂—N(R²⁶)—, —CH₂—O—CH₂—, *—CH₂—N(R²⁷)—(CH₂)—N(R²⁶)—, *—C≡C—CH₂—N(R²⁶)—, *—C≡C—(CH₂)₂—N(R²⁶)—*—C═C—CH₂—, *—C═C—CH₂—N(R²⁶)— 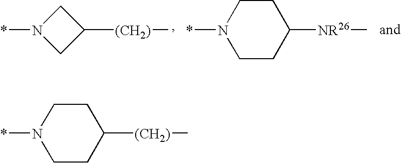
wherein * represents the point of attachment to ring A in formula I, and R²⁶ and R²⁷ is H or (1-3C)alkyl (suitably R²⁶ and R²⁷ are both H);

and wherein the group X-Z-Y optionally bears on carbon one or more substituent selected from hydroxy, (1-3C)alkyl, (1-3C)alkoxy, hydroxy-(1-3C)alkyl, (1-3C)alkoxy-(1-3C)alkyl, hydroxy-(2-3C)alkoxy and (1-3C)alkoxy(2-3C)alkoxy (for example X—Y-Z optionally bears on carbon 1 or 2 (1-3C)alkyl substituents).
(91) The group —X—Y-Z is selected from 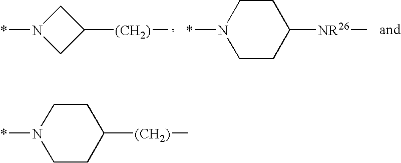
wherein * represents the point of attachment to ring A in formula I, and R²⁶ is H or (1-3C)alkyl (suitably R²⁶ is H);

and wherein the group X-Z-Y optionally bears on carbon one or more substituent selected from (1-3C)alkyl, hydroxy-(1-3C)alkyl, (1-3C)alkoxy-(1-3C)alkyl, hydroxy-(2-3C)alkoxy and (1-3C)alkoxy(2-3C)alkoxy (for example X—Y-Z optionally bears on carbon 1 or 2 (1-3C)alkyl substituents).

(92) The group —X—Y-Z is selected from —(CH₂)—C(O)—N(R²⁶)—*, —(CH₂)₂—C(O)—N(R²⁶)—*—(CH₂)₃—C(O)—N(R²⁶)—*, —(CH₂)—N(R²⁶)C(O)—*, —(CH₂)₂—N(R²⁶)C(O)—* and —(CH₂)₃—N(R²⁶)C(O)—,

wherein R²⁶ is H or (1-3C)alkyl (suitably R²⁶ is H);

and wherein the group X-Z-Y optionally bears on carbon one or more substituent selected from hydroxy, (1-3C)alkyl, (1-3C)alkoxy, hydroxy-(1-3C)alkyl, (1-3C)alkoxy-(1-3C)alkyl, hydroxy-(2-3C)alkoxy and (1-3C)alkoxy(2-3C)alkoxy (for example X—Y-Z optionally bears on carbon 1 or 2 (1-3C)alkyl substituents).

(93) The group —X—Y-Z is selected from —(CH₂)₃—, *—O—(CH₂)₂— and *—O—(CH₂)₃—NH—;

wherein * represents the point of attachment to ring A in formula I;

and wherein the group X-Z-Y optionally bears on carbon one or more substituent selected from (1-3C)alkyl.

(94) The group —X—Y-Z is —(CH₂)₃—, and wherein the group X-Z-Y optionally bears on carbon one or more substituent selected from (1-3C)alkyl.

(95) The group —X—Y-Z is *—O—(CH₂)₂; wherein * represents the point of attachment to ring A in formula I, and wherein the group X-Z-Y optionally bears on carbon one or more substituent selected from (1-3C)alkyl.

(96) The group —X—Y-Z is *—O—(CH₂)₃NH—; wherein * represents the point of attachment to ring A in formula I, and wherein the group X-Z-Y optionally bears on carbon one or more substituent selected from (1-3C)alkyl.

(97) R⁶ is a 5 or 6 membered monocyclic heteroaryl or a 9-, -10 or 11- (suitably 9- or 10-) membered bicyclic heteroaryl, which heteroaryl contains at least one —N═ ring atom and optionally 1 or more additional heteroatoms selected from O, S and N,

wherein R⁶ is linked to the group Z by a carbon atom in R⁶,

and wherein R⁶ optionally bears on carbon one or more R³¹ substituents,

and wherein if R⁶ contains an —NH— moiety, the nitrogen of said moiety optionally bears a group selected from R³⁵

and wherein:

(i) R⁶ is a 9-, -10 or 11- (suitably 9- or 10-) membered bicyclic fused heteroaryl which contains at least one unsubstituted —NH— ring member in addition to the —N═ ring atom in R⁶, wherein the —NH— and ═N— group in R⁶ are attached to the same bridgehead ring atom at a junction of the two fused rings in R⁶; or

(ii) R⁶ is substituted in an ortho position to the —N═ atom in R⁶ by an NHR^{31a} group; or

(iii) Z is NH and R⁶ is attached to Z by a ring carbon atom in an ortho position to the —N═ atom in R⁶;

and wherein the group Z-R⁶ has a pKa of greater than or equal to 6, such as from 6 to 12, for example from 6 to 9; and
wherein R³¹, R^{31a} and R³⁵ are as hereinbefore defined. Suitably in this embodiment the group R⁶-Z- is not 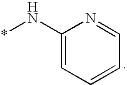
(98) R⁶ is linked to Z by a ring carbon atom in R⁶ and R⁶ is selected from any one of (a) to (f):

(a) imidazole which is substituted in an ortho position to the —N═ of the imidazole ring by —NHR^{31a};

(b) imidazole fused to: (bi) a monocyclic 6-membered aromatic, (bii) a monocyclic 5- or 6-membered heteroaromatic, (biii) a 3 to 7-membered heterocyclic or (biv) a (3-6C)cycloalkane ring,

and wherein R⁶ is substituted in the ortho position to the —N═ of the imidazole ring by —NHR^{31a};

(c) imidazole fused to a 5-, 6 or 7-membered heterocyclic, or to a monocyclic 5- or 6-membered heteroaromatic which heterocyclic or heteroaromatic contains at least one unsubstituted —NH— ring member and optionally contains 1 or 2 additional hetero atoms selected from O, S and N, and wherein the unsubstituted —NH— of the heterocyclic or heteroaromatic ring and the ═N— of the imidazole in R⁶ are attached to the same bridgehead ring atom at a junction of the two fused rings;

(d) pyridine which is substituted in an ortho position to the —N═ of the pyridine ring by —NHR^{31a};

(e) pyridine fused to (bi) a monocyclic 6-membered aromatic, (bii) a monocyclic 5- or 6-membered heteroaromatic, (biii) a 3 to 7-membered heterocyclic or (biv) a (3-6C)cycloalkane ring,

and wherein R⁶ is substituted in an ortho position to the —N═ of the pyridine ring by —NHR^{31a}; and

(f) pyridine fused to a 5-, 6 or 7-membered heterocyclic, or to a monocyclic 5- or 6-membered heteroaromatic which heterocyclic or heteroaromatic contains at least one unsubstituted —NH— ring member and optionally contains 1 or 2 additional hetero atoms selected from O, S and N, and wherein the unsubstituted —NH— of the heterocyclic ring and the ═N— of the pyridine ring in R⁶ are attached to the same bridgehead ring atom at a junction of the two fused rings;

or the group Z is NH, R⁶ is attached to Z by a carbon atom that is ortho to a —N=ring atom in R⁶ and R⁶ is selected from any one of (i) to (iv):

(i) imidazole;

(ii) imidazole fused to a 6-membered monocyclic aromatic, a 3- to 7-membered monocyclic heteroaromatic or heterocyclic ring or to a (3-6C)cycloalkane ring;

(iii) pyridine; and

(iv) pyridine fused 5- or 6-membered monocyclic aromatic, a 3- to 7-membered monocyclic heteroaromatic or heterocyclic ring or to a (3-6C)cycloalkane ring;

and wherein R⁶ optionally bears on carbon one or more R³¹ substituents,

and wherein if R⁶ contains an —NH— ring member, the nitrogen of said —NH— group optionally bears a group selected from R³⁵ (provided said —NH— group is not specified to be an unsubstituted —NH— above);

wherein R³¹, R^{31a} and R³⁵ are as hereinbefore defined. Suitably in this embodiment when X is O and Z is N(R²⁶), then R⁶ is not benzimidazolyl, particularly R⁶ is not benzimidazol-2-yl. Suitably R⁶ is linked to Z by a carbon atom in an aromatic ring in R⁶. Suitably in this embodiment the group R⁶-Z- is not 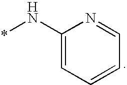
(99) R⁶ is linked to Z by a ring carbon atom in R⁶ and is selected from selected from any one of (a) to (f):

(a) imidazole which is substituted in an ortho position to the —N═ of the imidazole by —NHR^{31a};

(b) imidazole fused to a benzene, pyridine, pyrimidine, pyrazine, pyridazine or cyclohexane ring, and wherein R⁶ is substituted in an ortho position to the —N═ of the imidazole by —NHR^{31a};

(c) imidazole fused to a heterocyclic ring selected from morpholine, thiomorpholine, piperidine and piperazine, which heterocyclic ring carries at least one unsubstituted —NH— ring member, and wherein the unsubstituted —NH— of the heterocyclic ring and the ═N— of the imidazole ring in R⁶ are attached to the same bridgehead ring atom at a junction of the two fused rings;

(d) pyridine which is substituted in an ortho position to the —N═ of the pyridine by —NHR^{31a};

(e) pyridine fused to a benzene, 1,3-oxazole, isoxazole, furan, thiophene, 1,3-thiazole, isothiazole, pyrrole, pyridine, pyrimidine, pyrazine, cyclopentane or cyclohexane ring, and wherein R⁶ is substituted in an ortho position to the —N═ of the pyridine by —NHR^{31a}; and

(f) pyridine fused to a heterocyclic ring selected from morpholine, thiomorpholine, piperidine and piperazine, which heterocyclic ring carries at least one unsubstituted —NH— ring member, and wherein the unsubstituted —NH— of the heterocyclic ring and the ═N— of the imidazole ring in R⁶ are attached to the same bridgehead ring atom at a junction of the two fused rings;

or the group Z is NH, R⁶ is attached to Z by a carbon atom that is ortho to a —N=ring atom in R⁶ and R⁶ is selected from one of (i) to (iv):

(i) imidazole;

(ii) imidazole fused to a benzene, pyridine, pyrimidine, pyrazine, pyridazine or cyclohexane ring;

(iii) pyridine; and

(iv) pyridine fused to a benzene, 1,3-oxazole, isoxazole, furan, thiophene, 1,3-thiazole, isothiazole, pyrrole, pyridine, pyrimidine, pyrazine, cyclopentane or cyclohexane ring;

and wherein R⁶ optionally bears on carbon one or more R³¹ substituents,

and wherein if R⁶ contains an —NH— ring member, the nitrogen of said —NH— group optionally bears a group selected from R³⁵ (provided said —NH— group is not specified to be an unsubstituted —NH— above);

wherein R³¹, R^{31a} and R³⁵ are as hereinbefore defined. Suitably in this embodiment when X is O and Z is N(R²⁶), then R⁶ is not benzimidazolyl, particularly R⁶ is not benzimidazol-2-yl. Suitably R⁶ is linked to Z by a carbon atom in an aromatic ring in R⁶. Suitably in this embodiment the group R⁶-Z- is not 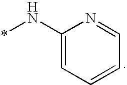
(100) R⁶ is as defined in any one of (97) to (99) wherein R⁶ is linked to the group —X—Y-Z- by a ring carbon atom, which ring carbon atom is ortho to a —N═ atom in R⁶.
(100a) R⁶ is as defined in any one of (97) to (100) wherein R⁶ is linked to the group —X—Y-Z- by a ring carbon atom which carbon atom is located in an aromatic ring in R⁶.
(101) R⁶ is imidazol-2-yl, which is substituted at the 5-position on the imidazolyl ring by —NHR^{31a} or R⁶ is pyridin-2-yl, which is substituted at the 6-position on the pyridyl ring by —NHR^{31a};

and wherein R⁶ optionally bears on carbon one or more R³¹ substituents,

and wherein the —NH— of the imidazol-2-yl ring optionally bears a group selected from R³⁵;

wherein R³¹, R^{31a} and R³⁵ are as hereinbefore defined.

(102) Z is NH and R⁶ is benzimidazol-2-yl and wherein R⁶ optionally bears on carbon one or more R³¹ substituents,

and wherein the —NH— of the benzimidazol-2-yl ring optionally bears a group selected from R³⁵;

wherein R³¹, R^{31a} and R³⁵ are as hereinbefore defined;

(103) R⁶ is as defined in any one of (97) to (102) wherein and wherein R⁶ optionally bears on carbon one or more R³¹ substituents selected from amino, (1-4C)alkyl, (1-4C)alkoxy, (1-4C)alkylsulfonyl, (1-4C)alkylamino and di-[(1-4C)alkyl]amino,

or from a group of the formula:
—X⁸—R³²
wherein X⁸ is a direct bond, O or N(R³³), wherein R³³ is hydrogen or (1-4C)alkyl, and R³² is hydroxy-(1-4C)alkyl, (1-4C)alkoxy-(1-4C)alkyl, amino-(1-4C)alkyl, (1-4C)alkylamino-(1-4C)alkyl and di-[(1-4C)alkyl]amino-(1-4C)alkyl,

or from a group of the formula:
—X⁹-Q⁶

wherein X⁹ is a direct bond, O or N(R³⁴), wherein R³⁴ is hydrogen or (1-4C)alkyl, and Q⁶ is (3-6C)cycloalkyl or (3-6C)cycloalkyl-(1-4C)alkyl,

and wherein if R⁶ contains an —NH— ring member, the nitrogen of said —NH— group optionally bears an R³⁵ group selected from (1-4C)alkyl, hydroxy-(2-4C)alkyl, (1-4C)alkoxyalkyl, amino-(2-4C)alkyl, (1-4C)alkylamino-(2-4C)alkyl, di-[(1-4C)alkyl]amino-(2-4C)alkyl, (3-6C)cycloalkyl and (3-6C)cycloalkyl-(1-4C)alkyl,

and wherein R^{31a} is selected from hydrogen, (1-6C)alkyl, (2-8C)alkenyl, (2-8C)alkynyl, halo-(1-4C)alkyl, hydroxy-(2-4C)alkyl, (1-4C)alkoxy-(2-4C)alkyl, amino-(2-4C)alkyl, (1-4C)alkylamino-(2-4C)alkyl, di-[(1-4C)alkyl]amino-(2-4C)alkyl, (3-6C)cycloalkyl and (3-6C)cycloalkyl-(1-4C)alkyl, and wherein any (3-6C)cycloalkyl in R^{31a} optionally bears 1 or more (for example 1 or 2) (1-4C)alkyl substituents.

(104) R⁶ is as defined in any one of (97) to (102) wherein and wherein R⁶ optionally bears on carbon one or more R³¹ substituents selected from amino, (1-4C)alkyl, (1-4C)alkoxy, (1-4C)alkylamino and di-[(1-4C)alkyl]amino,

or from a group of the formula:
—X⁹-Q⁶

wherein X⁹ is a direct bond, O or N(R³⁴), wherein R³⁴ is hydrogen or (1-4C)alkyl, and Q⁶ is (3-6C)cycloalkyl or (3-6C)cycloalkyl-(1-4C)alkyl,

and wherein if R⁶ contains an —NH— ring member, the nitrogen of said —NH— group optionally bears an R³⁵ group selected from (1-4C)alkyl, (3-6C)cycloalkyl and (3-6C)cycloalkyl-(1-4C)alkyl,

and wherein R^{31a} is selected from hydrogen, (1-4C)alkyl, amino-(2-4C)alkyl, (1-4C)alkylamino-(2-4C)alkyl, di-[(1-4C)alkyl]amino-(2-4C)alkyl, (3-6C)cycloalkyl and (3-6C)cycloalkyl-(1-4C)alkyl, and wherein any (3-6C)cycloalkyl in R^{31a} optionally bears 1 or more (for example 1 or 2) (1-4C)alkyl substituents.

(105) R⁶ is heteroaryl, which heteroaryl is a monocyclic or bicyclic heteroaryl that contains at least one nitrogen atom, and wherein R⁶ optionally bears on carbon one or more R³¹ substituents selected from amino, (1-4C)alkyl, (1-4C)alkoxy, (1-4C)alkylamino and di-[(1-4C)alkyl]amino,

or from a group of the formula:
—X⁸—R³²
wherein X⁸ is a direct bond or is N(R³³), wherein R³³ is hydrogen or (1-4C)alkyl, and R³² is hydroxy-(1-4C)alkyl, (1-4C)alkoxy-(1-4C)alkyl, amino-(1-6C)alkyl, (1-6C)alkylamino-(1-6C)alkyl and di-[(1-6C)alkyl]amino-(1-6C)alkyl,

or from a group of the formula:
—X⁹-Q⁶

wherein X⁹ is a direct bond or is N(R³⁴), wherein R³⁴ is hydrogen or (1-4C)alkyl, and Q⁶ is (3-6C)cycloalkyl or (3-6C)cycloalkyl-(1-4C)alkyl,
and wherein if R⁶ contains an —NH— moiety, the nitrogen of said moiety optionally bears a group selected from (1-4C)alkyl. Suitably in this embodiment the group R⁶-Z- is not 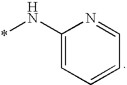
(106) R⁶ is a 5 or 6 membered monocyclic heteroaryl, or a 5,6 or 6,6 bicyclic heteroaryl, which heteroaryl contains at least one nitrogen atom and optionally 1 or more (for example 1, 2, 3 or 4) additional heteroatoms selected from O, S and N, and wherein R⁶ optionally bears on carbon one or more R³¹ substituents selected from amino, (1-4C)alkyl, (1-4C)alkylamino and di-[(1-4C)alkyl]amino,

or from a group of the formula:
—X⁸—R³²
wherein X⁸ is a direct bond or is N(R³³), wherein R³³ is hydrogen or (1-4C)alkyl, and R³² is hydroxy-(1-4C)alkyl, (1-4C)alkoxy-(1-4C)alkyl, amino-(1-6C)alkyl, (1-6C)alkylamino-(1-6C)alkyl and di-[(1-6C)alkyl]amino-(1-6C)alkyl,

or from a group of the formula:
—X⁹-Q⁶

wherein X⁹ is a direct bond or is N(R³⁴), wherein R³⁴ is hydrogen or (1-4C)alkyl, and Q⁶ is (3-6C)cycloalkyl or (3-6C)cycloalkyl-(1-4C)alkyl,
and wherein if R⁶ contains an —NH— moiety, the nitrogen of said moiety optionally bears a group selected from (1-4C)alkyl. Suitably in this embodiment the group R⁶-Z- is not 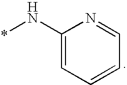
(107) R⁶ is a heteroaryl as described in any of (97) to (102), which heteroaryl is attached to the group Z in a position ortho to a nitrogen atom in R⁶.
(108) R⁶ is selected from: 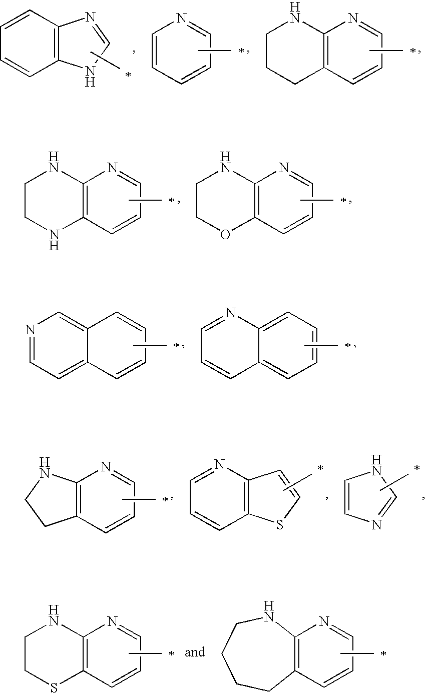
wherein * indicates the point of attachment of R⁶ to the group Z in formula I,
and wherein the R⁶ groups are optionally substituted as defined in any one of (97) to (106).
Suitably in this embodiment the group R⁶-Z- is not 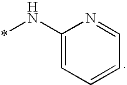
(110) R⁶ is selected from: 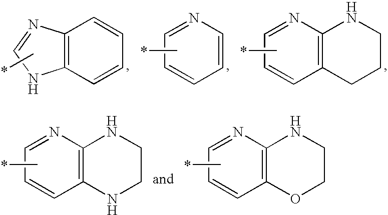
wherein * indicates the point of attachment of R⁶ to the group Z in formula I,
and wherein the R⁶ groups are optionally substituted as defined in any one of (97) to (106).
Suitably in this embodiment the group R⁶-Z- is not 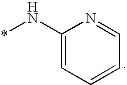
(111) R⁶ is selected from:

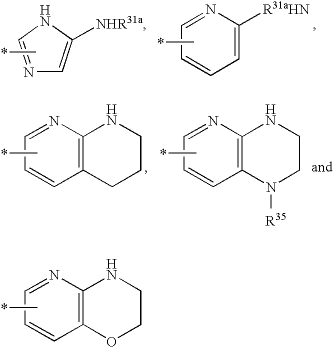

wherein * indicates the point of attachment of R⁶ to the group Z in formula I, and R⁶ is attached to the group —X—Y-Z- by a carbon atom in an aromatic ring of R⁶;

R^{31a} is as hereinbefore defined, for example as in any of (103) to (104) above;

R³⁵ is as hereinbefore defined, for example R³⁵ is (1-4C)alkyl, (3-6C)cycloalkyl and (3-6C)cycloalkyl-(1-4C)alkyl;

and wherein the R⁶ groups are optionally substituted on a ring carbon by one of more R³¹ as hereinbefore defined, for example as described in any one of (103) to (104) above.

(111a) R⁶ is as defined in any one of (105) to (111) wherein R⁶ is linked to the group —X—Y-Z- by a ring carbon atom, which carbon atom is located in an aromatic ring in R⁶.

(112) Z is NH and the group R⁶-Z- is selected from: 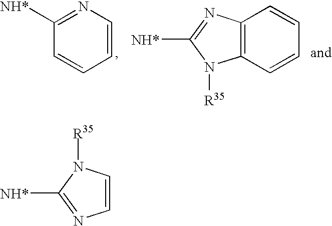
wherein * indicates the point of attachment of R⁶-Z- to Y in formula I,

R³⁵ is as hereinbefore defined, for example R³⁵ is hydrogen, (1-4C)alkyl, (3-6C)cycloalkyl and (3-6C)cycloalkyl-(1-4C)alkyl;

and wherein the R⁶ groups are optionally substituted on a ring carbon by one of more R³¹ as hereinbefore defined, for example as described in any one of (103) to (104) above.

Suitably in this embodiment the group R⁶-Z- is not 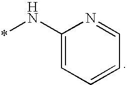
(113) R⁶ is selected from:

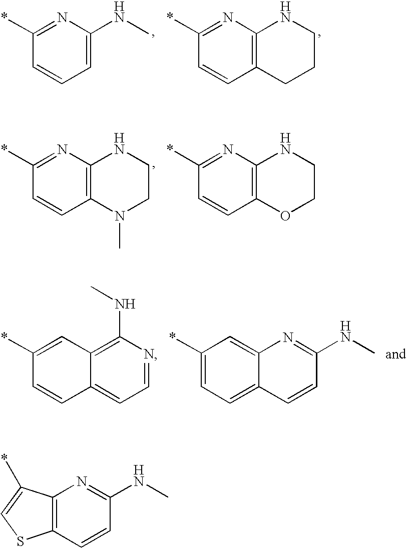

or Z is N(R²⁶), wherein R²⁶ is as hereinbefore defined (for example R²⁶ is hydrogen) and the group R⁶-Z- is selected from:

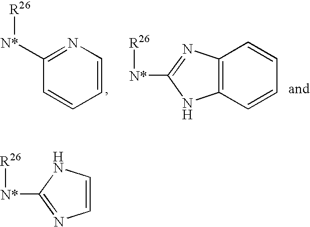

wherein * indicates the point of attachment of R⁶ to the group Z or of R⁶-Z- to Y in formula I,

and wherein the R⁶ groups are optionally substituted as defined in any one of (97) to (102).

Suitably in this embodiment the group R⁶-Z- is not 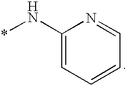
(114) R⁶ is selected from:

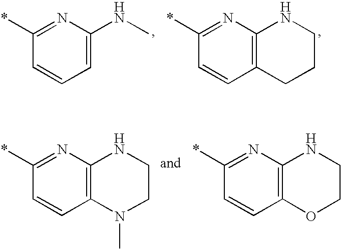

or Z is N(R²⁶), wherein R²⁶ is as hereinbefore defined (for example R²⁶ is hydrogen) and R⁶-Z- is selected from:

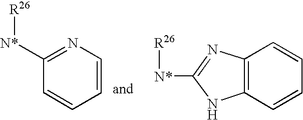

wherein * indicates the point of attachment of R⁶ to the group Z or R⁶-Z- to Y in formula I,

and wherein the R⁶ groups are optionally substituted as defined in any one of (97) to (106).

Suitably in this embodiment the group R⁶-Z- is not 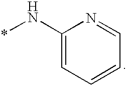
(115) R⁶ is selected from:

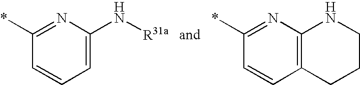

wherein R^{31a} is as hereinbefore defined, for example as defined in any one of (103) or (104) above. Particularly R^{31a} is selected from hydrogen (1-3C)alkyl, (3-6C)cycloalkyl, or (3-6C)cycloalkyl-(1-3C)alkyl, more particularly R^{31a} is (1-3C)alkyl, cyclopropyl or cyclopropylmethyl, for example R^{31a} is methyl;

* indicates the point of attachment of R⁶ to the group Z in formula I;

and wherein the R⁶ groups are optionally substituted on carbon by R³¹ as defined in any one of (97) to (102). In a particular embodiment R⁶ is not substituted by R³¹. In another particular embodiment R⁶ is 5,6,7,8-tetrahydro-1,8-naphthyridin-2-yl which optionally bears at the 6-, 7- or 8-positions one or more R³¹ substituents as hereinbefore define, for example as defined in any one of (97) to (102), particularly R³¹ is (1-3C)alkyl, (3-6C)cycloalkyl and (3-6C)cycloalkyl-(1-3C)alkyl, (for example R³¹ is methyl or methoxy).

(116) R⁶ is selected from:

(i) pyridin-2-yl, which is substituted at the 6-position by —NHR^{31a};

(ii) 3,4-dihydro-2H-pyrido[3,2-b][1,4]oxazin-6-yl;

(iii) quinolin-7-yl, which is substituted at the 6-position by —NHR^{31a};

(iv) 1,2,3,4-tetrahydropyrido[2,3-b]pyrazin-6-yl; and

(v) 5,6,7,8-tetrahydro-1,8-naphthyridin-2-yl, and 5,6,7,8-tetrahydro-1,8-naphthyridin-2-yl;

or Z is NH and the group R⁶-Z- is selected from 1H-benzimidazol-2-ylamino and pyridin-2-ylamino;

wherein R^{31a} is as hereinbefore defined, for example as defined in any one of (103) or (104) above, more particularly R^{31a} is (1-3C)alkyl, cyclopropyl or cyclopropylmethyl, for example R^{31a} is methyl;
and wherein the R⁶ groups are optionally substituted on carbon by R³¹ wherein R³¹ is selected from (1-3C)alkyl, (3-6C)cycloalkyl, (3-6C)cycloalkyl-(1-3C)alkyl and (1-3Calkoxy) such as methyl or methoxy. Suitably in this embodiment the group R⁶-Z- is not 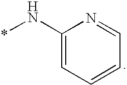
(117) R⁶ is selected from:
6-aminopyridin-2-yl, 6-(cyclopentylamino)pyridin-2-yl, 6-(cyclopropylamino)pyridin-2-yl, 6-[(cyclopropylmethyl)amino]pyridin-2-yl, 3,4-dihydro-2H-pyrido[3,2-b][1,4]oxazin-6-yl, 6-{[2-(dimethylamino)ethyl]amino}pyridin-2-yl, 2-methoxyethyl)amino]pyridin-2-yl, 5-methoxy-6-(methylamino)pyridin-2-yl, 6-(dimethylamino)pyridin-2-yl, 6-(methylamino)pyridin-2-yl, 2-(methylamino)quinolin-7-yl, 1-methyl-1,2,3,4-tetrahydropyrido[2,3-b]pyrazin-6-yl, 4-methyl-5,6,7,8-tetrahydro-1,8-naphthyridin-2-yl, 5-methyl-5,6,7,8-tetrahydro-1,8-naphthyridin-2-yl, 7-methyl-5,6,7,8-tetrahydro-1,8-naphthyridin-2-yl and 5,6,7,8-tetrahydro-1,8-naphthyridin-2-yl;

or Z is NH and the group R⁶-Z- is selected from:
1H-benzimidazol-2-ylamino, 4-ethylpyridin-2-ylamino, 5-methylpyridin-2-ylamino, 4-methylpyridin-2-ylamino, 6-methylpyridin-2-ylamino, 3-methylpyridin-2-ylamino, 4,6-dimethylpyridin-2-ylamino, 4-methoxypyridin-2-yl)amino, 5-methoxypyridin-2-ylamino, and pyridin-2-ylamino. Suitably in this embodiment the group R⁶-Z- is not 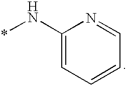
(118) R⁶ is selected from:

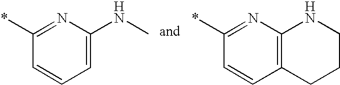

wherein * indicates the point of attachment of R⁶ to the group Z in formula I;

and wherein the R⁶ groups are optionally substituted on carbon by R³¹ as defined hereinbefore, for example R³¹ is selected from (1-3C)alkyl, (3-6C)cycloalkyl, (3-6C)cycloalkyl-(1-3C)alkyl and (1-3Calkoxy) such as methyl or methoxy.

Suitably, however, these R⁶ groups are not substituted by R³¹.

(119) R⁶ is as defined in (114) to (118) and the group X—Y-Z- is as defined in any one of (89) to (96).

(120) R⁶ is 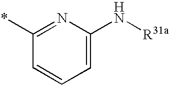
wherein R^{31a} is selected from hydrogen (1-3C)alkyl, (3-6C)cycloalkyl, or (3-6C)cycloalkyl-(1-3C)alkyl, more particularly R^{31a} is (1-3C)alkyl, cyclopropyl or cyclopropylmethyl, for example R^{31a} is methyl;
* indicates the point of attachment of R⁶ to the group Z in formula I; and
the group —X—Y-Z- is —(CH₂)₃— or **—O—(CH₂)₂—; wherein ** represents the point of attachment to ring A in formula I.
(120) R⁶ is 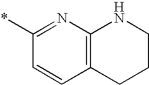
wherein * indicates the point of attachment of R⁶ to the group Z in formula I; and the group —X—Y-Z- is —(CH₂)₃— or **—O—(CH₂)₂—; wherein ** represents the point of attachment to ring A in formula I.
(121) R⁶ is 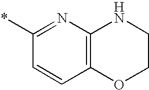
wherein * indicates the point of attachment of R⁶ to the group Z in formula I; and the group —X—Y-Z- is —(CH₂)₃— or **—O—(CH₂)₂—; wherein ** represents the point of attachment to ring A in formula I.
(122) X, Y, Z and R⁶ have any of the values defined herein and the chain length between ring A and a ring —N═ in R⁶ is 4 or 5, particularly 5 atoms long;

or when Z is NH and R⁶ is attached to Z by a carbon atom that is in an ortho position to a —N═ ring atom in R⁶, then the chain length of the group —X—Y-Z- is 4 or 5 (particularly 5) atoms long.

(123) X, Y, Z and R⁶ have any of the values defined herein, and the chain length between ring A and a ring —N═ in R⁶ is 4 or 5 (particularly 5) atoms long.

(124) X^{a} is oxygen.

In an embodiment of the invention there is provided a compound of the formula I wherein:

R¹ is selected from chloro methyl and ethyl (for example R¹ is chloro or methyl);

R² is selected from hydrogen, fluoro, chloro, bromo and methyl;

m is 0 or 1 and R³ is selected from halo, (1-3C)alkyl and (1-3C)alkoxy,

or R² and an adjacent R³ substituent optionally form a methylenedioxy group;
Ring A is selected from: 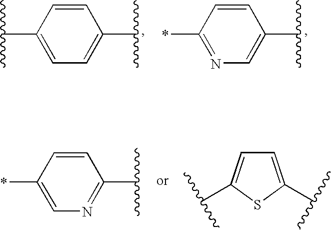
wherein * shows the point of attachment to X in formula I and wherein A is optionally substituted by n R⁵ groups;

X^{a} is oxygen or sulfur (particularly X^{a} is oxygen);

R⁴ is hydrogen;

n is 0, 1 or 2 and each R⁵, which may be the same or different, is selected from halo, amino, (1-4C)alkyl, (1-4C)alkoxy (1-4C)alkylamino and di-[(1-4C)alkyl]amino; the group —X—Y-Z- is selected from —(CH₂)₃— and *—O—(CH₂)₂—,

wherein * represents the point of attachment to ring A in formula I,

and wherein the group X-Z-Y optionally bears on carbon one or more substituent selected from (1-3C)alkyl; and

R⁶ is selected from

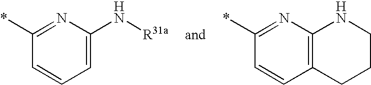

wherein R^{31a} is as defined in claim 1;

* indicates the point of attachment of R⁶ to the group Z in formula I;

and wherein R⁶ is optionally substituted on carbon by R³¹ is as defined hereinbefore, for example R³¹ is (1-3C)alkyl such as methyl;

or a pharmaceutically acceptable salt or prodrug thereof.

In this embodiment when ring A is: 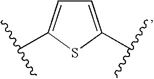
then —X—Y-Z- is suitably —(CH₂)₃—.

In another embodiment of the invention there is provided a compound of the formula I as hereinbefore defined wherein:

X^{a} is O;

R⁴ is hydrogen;

Ring A is selected from:

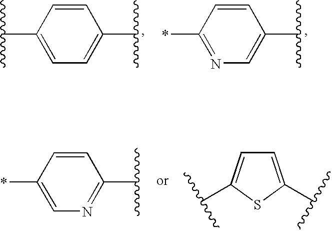

—X—Y-Z- is —(CH₂)₃— or *—O(CH₂)₂—, wherein * indicates the point of attachment of —X—Y-Z- to Ring A;

R⁶ is selected from: 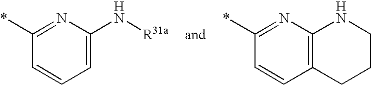
wherein R^{31a} has any of the values defined herein, particularly R^{31a} is methyl; and the group: 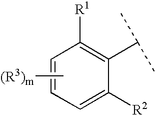
in formula I is as defined in any one of paragraphs (35), (35a), (35b), (36) or (36a) above;
or a pharmaceutically acceptable salt or prodrug thereof.

In this embodiment when ring A is: 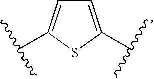
then —X—Y-Z- is suitably —(CH₂)₃—.

In another embodiment of the invention there is provided a compound of the formula I which is of the formula IA:

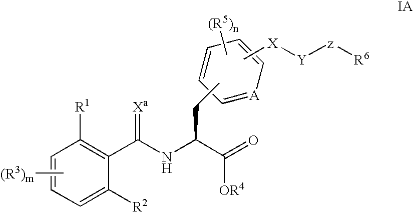

wherein:

A is Nor CH, and n, m, R¹, R², R³, R⁴, R⁵, R⁶, X^{a}, X, Y and Z have any of the values hereinbefore defined;

or a pharmaceutically acceptable salt or prodrug thereof,

provided that the compound of formula I is not 4-{[(2R)-2-amino-3-(3-pyridyl)-1-oxopropyl]amino}-N-[(2-ethyl-6-methylphenyl)thioxomethyl]-L-phenylalanine methyl ester.

In another embodiment of the invention there is provided a compound of the formula I which is of the formula IA as hereinbefore defined wherein:

X^{a} is oxygen;

A is N or CH;

R¹ is selected from chloro, methyl and ethyl, for example R¹ is chloro or methyl;

R² is selected from hydrogen, halo and (1-4C)alkyl, for example R² is selected from hydrogen, fluoro, chloro and methyl;

m is 0 or 1 and each R³, which may be the same or different, is selected from halo and (1-4C)alkyl, or two R³ substituents optionally form a (1-3C)alkylenedioxy group, for example each R³, which may be the same or different, is selected from fluoro, chloro, bromo and (1-3C)alkyl;

R⁴ is selected from hydrogen and (1-4C)alkyl, wherein R⁴ is optionally bears on carbon a hydroxy or (1-4C)alkoxy substituent, for example R⁴ is hydrogen;

n is 0, 1 or 2 and each R⁵, which may be the same or different, is selected from halo, trifluoromethyl, cyano, hydroxy, amino, (1-4C)alkyl, (2-4C)alkenyl, (2-4C)alkynyl, (1-4C)alkoxy (1-4C)alkylamino and di-[(1-4C)alkyl]amino,

or from a group of the formula:
Q⁵-X⁷—
wherein X⁷ is a direct bond or is selected from O and N(R²³) wherein R²³ is hydrogen or (1-4C)alkyl, and Q⁵ is (3-6C)cycloalkyl, (3-6C)cycloalkyl-(1-4C)alkyl, heterocyclyl or heterocyclyl-(1-4C)alkyl, which heterocyclyl is a saturated monocyclic heterocyclyl group containing 1 or 2 heteroatoms selected from O, S and N,

and wherein R⁵ optionally bears on carbon one or more R²⁴ substituents selected from halo, cyano, hydroxy, amino, (1-4C)alkoxy, (1-4C)alkylamino and di-[(1-4C)alkyl]amino,

and wherein if any heteroaryl or heterocyclyl group within R⁵ contains an —NH-moiety, the nitrogen of said moiety optionally bears an R²⁵ selected from (1-4C)alkyl, (1-4C)alkylsulfonyl and (2-4C)alkanoyl,

or from a group of the formula:
—X⁶-Q⁴

wherein X⁶ is a direct bond or is selected from C(O) and SO₂, and Q⁴ is (3-7C)cycloalkyl or (3-7C)cycloalkyl-(1-6C)alkyl;

and wherein any heterocyclyl group within R⁵ optionally bears 1 oxo substituent;

for example n is 0, 1 or 2 and each R⁵, which may be the same or different, is selected from halo, hydroxy, amino, (1-4C)alkyl, (1-4C)alkoxy (1-4C)alkylamino and di-[(1-4C)alkyl]amino,

and wherein R⁵ optionally bears on carbon one or more R²⁴ substituents selected from halo, hydroxy, amino, (1-4C)alkoxy, (1-4C)alkylamino and di-[(1-4C)alkyl]amino;

X is selected from a direct bond, N(R²⁶), O, C(O)N(R²⁶), N(R²⁶)C(O), (1-6)alkylene, CH═CH and C≡C, wherein R²⁶ is hydrogen, (1-6C)alkyl or (3-7C)cycloalkyl,

Z is selected from a direct bond, N(R²⁶), O, (1-6)alkylene, CH═CH and C≡C,

and wherein any X and Z optionally bears on carbon one or more R²⁸ substituents wherein R²⁸ is as hereinbefore defined;

Y is selected from (1-4C)alkylene, (3-6C)cycloalkylene and a monocyclic heterocyclyl which contains 1 or 2 heteroatoms selected from O, S and N, and wherein adjacent carbon atoms in any (2-4C)alkylene chain within a Y substituent are optionally separated by the insertion into the chain of a group selected from 00 N(R²⁷), N(R²⁷)C(O), C(O)N(R²⁷), CH═CH and C≡C, wherein R²⁷ is hydrogen, (1-4C)alkyl or (3-6C)cycloalkyl,

and wherein Y optionally bears on carbon one or more R²⁸ substituents as hereinbefore defined,

and wherein if any heterocyclyl group within Y contains an —NH— moiety, the nitrogen of said moiety optionally bears a group selected from R²⁹, as hereinbefore defined

provided that when X is a direct bond or O and Y is a heterocyclyl containing a nitrogen heteroatom, then the group R⁶-Z- is attached to a carbon atom in the heterocyclyl containing the nitrogen heteroatom;

R⁶ is a 5 or 6 membered monocyclic heteroaryl, or a 5,6 or 6,6 bicyclic heteroaryl, which heteroaryl contains at least one nitrogen atom and optionally 1 or more (for example 1, 2, 3 or 4) additional heteroatoms selected from O, S and N, and wherein R⁶ optionally bears on carbon one or more R³¹ substituents selected from amino, (1-4C)alkyl, (1-4C)alkylamino and di-[(1-4C)alkyl]amino,

or from a group of the formula:
—X⁹-Q⁶

wherein X⁹ is a direct bond or is N(R³⁴), wherein R³⁴ is hydrogen or (1-4C)alkyl, and Q⁶ is (3-6C)cycloalkyl or (3-6C)cycloalkyl-(1-4C)alkyl,

and wherein if R⁶ contains an —NH— moiety, the nitrogen of said moiety optionally bears a group selected from (1-4C)alkyl, (for example R⁶ is one of the groups defined in any of (97) to (118) above);

or a pharmaceutically acceptable salt or prodrug thereof.

In another embodiment of the invention there is provided a compound of the formula I which is of the formula IA as hereinbefore defined wherein:

A is N or CH, and n, m, R¹, R², R³, R⁴, R⁵ and R⁶ have any of the values hereinbefore defined;

X^{a} is oxygen;

X is selected from a direct bond, N(R²⁶), O, C(O)N(R²⁶), N(R²⁶)C(O) and C≡C, wherein R²⁶ is hydrogen, (1-6C)alkyl or (3-7C)cycloalkyl;

Y is selected from (1-4C)alkylene, and wherein Y optionally bears on carbon one or more R²⁸ substituents selected from (1-3C)alkyl; and

Z is selected from a direct bond and NR²⁶ wherein R²⁶ is hydrogen, (1-4C)alkyl or (3-6C)cycloalkyl;

or a pharmaceutically acceptable salt or prodrug thereof.

In the compounds of formula IA a particular value for R⁴ is hydrogen.

In another embodiment of the invention there is provided a compound of the formula I which is of the formula IA as hereinbefore defined wherein:

X^{a} is O or S (particularly O);

R¹ is selected from chloro, bromo, methyl, ethyl, cyclopropyl, cyclobutyl and trifluoromethyl (particularly R¹ is chloro or methyl);

R² is selected from hydrogen, fluoro, chloro, bromo, methyl, ethyl, cyclopropyl, cyclobutyl and trifluoromethyl (particularly R² is selected from hydrogen, fluoro, chloro and methyl, more particularly R² is methyl or fluoro);

m and R³ are as defined in any one of paragraphs (25) to (33) above;

R⁴ is hydrogen;

n is 0, 1 or 2 (suitably n is 0) and each R⁵, which may be the same or different, is as defined in any of (47) to (49), for example each R⁵, is selected from halo, amino, (1-4C)alkyl, (1-4C)alkoxy, (1-4C)alkylamino and di-[(1-4C)alkyl]amino,

and wherein R⁵ optionally bears on carbon one or more R²⁴ substituents selected from halo, hydroxy, amino, (1-4C)alkoxy, (1-4C)alkylamino and di-[(1-4C)alkyl]amino;

the group —X—Y-Z- is as defined in any one of paragraphs (93) to (96) above (for example —X—Y-Z- is —(CH₂)₃— or *—O(CH₂)₂—, wherein * indicates the point of attachment of —X—Y-Z- to the ring containing A);

R⁶ is as defined in any one of paragraphs (97) to (118) (particularly R⁶ is as defined in any one of paragraphs (114) to (118), more particularly wherein R⁶ is attached to the group —X—Y-Z- by a ring carbon atom in R⁶, which carbon atom is located in an aromatic ring in R⁶);

or a pharmaceutically acceptable salt or prodrug thereof.

In a further embodiment in the compounds of formula (IA) defined above, the chain length between the ring containing A and a ring —N═ in R⁶ is 4 or 5, particularly 5 atoms long;

or when Z is NH and R⁶ is attached to Z by a carbon atom that is in an ortho position to a —N═ ring atom in R⁶, then the chain length of the group —X—Y-Z- is 4 or 5 (particularly 5) atoms long.

In another embodiment of the invention there is provided a compound of the formula I which is of the formula IB:

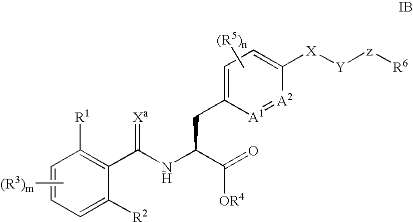

wherein:

A¹ and A² are selected from N and CH, provided that A¹ and A² are not both N;

and n, m, R¹, R², R³, R⁴, R⁵, R⁶, X^{a}, X, Y and Z have any of the values hereinbefore defined;

or a pharmaceutically acceptable salt or prodrug thereof.

In another embodiment of the invention there is provided a compound of the formula I which is of the formula IB as hereinbefore defined wherein X^{a} is oxygen, one of A¹ and A² is N and the other of A¹ and A² is CH.

In another embodiment of the invention there is provided a compound of the formula I which is of the formula IB as hereinbefore defined wherein X^{a} is oxygen, A¹ is N and A² is CH.

In another embodiment of the invention there is provided a compound of the formula I which is of the formula IB as hereinbefore defined wherein X^{a} is oxygen, A¹ is CH and A² is N.

In another embodiment of the invention there is provided a compound of the formula I which is of the formula IB as hereinbefore defined wherein:

X^{a} is oxygen;

A¹ and A² are selected from N and CH, provided that A¹ and A² are not both N;

R¹ is selected from chloro, methyl and ethyl, for example R¹ is chloro or methyl;

R² is selected from hydrogen, halo and (1-4C)alkyl, for example R² is selected from hydrogen, fluoro, chloro and methyl;

m is 0 or 1 and each R³, which may be the same or different, is selected from halo and (1-4C)alkyl, or two R³ substituents optionally form a (1-3C)alkylenedioxy group, for example each R³, which may be the same or different, is selected from fluoro, chloro, bromo and (1-3C)alkyl;

R⁴ is selected from hydrogen and (1-4C)alkyl, wherein R⁴ is optionally bears on carbon a hydroxy or (1-4C)alkoxy substituent, for example R⁴ is hydrogen;

n is 0, 1 or 2 and each R⁵, which may be the same or different, is selected from halo, hydroxy, amino, (1-4C)alkyl, (1-4C)alkoxy (1-4C)alkylamino and di-[(1-4C)alkyl]amino,

and wherein R⁵ optionally bears on carbon one or more R²⁴ substituents selected from halo, hydroxy, amino, (1-4C)alkoxy, (1-4C)alkylamino and di-[(1-4C)alkyl]amino;

X is selected from a direct bond, N(R²⁶), O, C(O)N(R²⁶), N(R²⁶)C(O), (1-6)alkylene, CH═CH and C≡C, wherein R²⁶ is hydrogen, (1-6C)alkyl or (3-7C)cycloalkyl,

Z is selected from a direct bond, N(R²⁶), 0, (1-6)alkylene, CH═CH and C≡C,

and wherein any X and Z optionally bears on carbon one or more R²⁸ substituents wherein R²⁸ is as hereinbefore defined;

Y is selected from (1-4C)alkylene, (3-6C)cycloalkylene and a monocyclic heterocyclyl which contains 1 or 2 heteroatoms selected from O, S and N, and wherein adjacent carbon atoms in any (2-4C)alkylene chain within a Y substituent are optionally separated by the insertion into the chain of a group selected from O, N(R²⁷), N(R²⁷)C(O), C(O)N(R²⁷), CH═CH and C≡C, wherein R²⁷ is hydrogen, (1-4C)alkyl or (3-6C)cycloalkyl,

and wherein Y optionally bears on carbon one or more R²⁸ substituents as hereinbefore defined,

and wherein if any heterocyclyl group within Y contains an —NH— moiety, the nitrogen of said moiety optionally bears a group selected from R²⁹, as hereinbefore defined

provided that when X is a direct bond or O and Y is a heterocyclyl containing a nitrogen heteroatom, then the group R⁶-Z- is attached to a carbon atom in the heterocyclyl containing the nitrogen heteroatom;

R⁶ is a 5 or 6 membered monocyclic heteroaryl, or a 5,6 or 6,6 bicyclic heteroaryl, which heteroaryl contains at least one nitrogen atom and optionally 1 or more (for example 1, 2 or 3) additional heteroatoms selected from O, S and N, and wherein R⁶ optionally bears on carbon one or more R³¹ substituents selected from amino, (1-4C)alkyl, (1-4C)alkylamino and di-[(1-4C)alkyl]amino,

or from a group of the formula:
—X⁹-Q⁶
wherein X⁹ is a direct bond or is N(R³⁴), wherein R³⁴ is hydrogen or (1-4C)alkyl, and Q⁶ is (3-6C)cycloalkyl or (3-6C)cycloalkyl-(1-4C)alkyl,

and wherein if R⁶ contains an —NH— moiety, the nitrogen of said moiety optionally bears a group selected from (1-4C)alkyl, (for example R⁶ is one of the groups defined in any of (97) to (118) above);

or a pharmaceutically acceptable salt or prodrug thereof.

In the compounds of formula IB a particular value for R⁴ is hydrogen.

A particular value for X^{a} in compounds of formula (IB) is oxygen.

In another embodiment of the invention there is provided a compound of the formula I which is of the formula IB as hereinbefore defined wherein:

one of A¹ and A² is N and the other of A¹ and A² is CH;

X^{a} is O or S (particularly O);

R¹ is selected from chloro, bromo, methyl, ethyl, cyclopropyl, cyclobutyl and trifluoromethyl (particularly R¹ is chloro or methyl);

R² is selected from hydrogen, fluoro, chloro, bromo, methyl, ethyl, cyclopropyl, cyclobutyl and trifluoromethyl (particularly R² is selected from hydrogen, fluoro, chloro and methyl, more particularly R² is methyl or fluoro);

m and R³ are as defined in any one of paragraphs (25) to (33) above;

R⁴ is hydrogen;

n is 0, 1 or 2 (suitably n is 0) and each R⁵, which may be the same or different, is as defined in any of (47) to (49), for example each R⁵, is selected from halo, amino, (1-4C)alkyl, (1-4C)alkoxy, (1-4C)alkylamino and di-[(1-4C)alkyl]amino,

and wherein R⁵ optionally bears on carbon one or more R²⁴ substituents selected from halo, hydroxy, amino, (1-4C)alkoxy, (1-4C)alkylamino and di-[(1-4C)alkyl]amino;

the group —X—Y-Z- is as defined in any one of paragraphs (93) to (96) above (for example —X—Y-Z- is —(CH₂)₃— or *—O(CH₂)₂—, wherein * indicates the point of attachment of —X—Y-Z- to the ring containing A¹ and A²);

R⁶ is as defined in any one of paragraphs (97) to (118) (particularly R⁶ is as defined in any one of paragraphs (114) to (118) more particularly wherein R⁶ is attached to the group —X—Y-Z- by a ring carbon atom in R⁶, which carbon atom is located in an aromatic ring in R⁶);

or a pharmaceutically acceptable salt or prodrug thereof.

In a further embodiment in the compounds of formula (IB) defined above, the chain length between the ring containing A¹ and A² and a ring —N═ in R⁶ is 4 or 5, particularly 5 atoms long;

or when Z is NH and R⁶ is attached to Z by a carbon atom that is in an ortho position to a —N═ ring atom in R⁶, then the chain length of the group —X—Y-Z- is 4 or 5 (particularly 5) atoms long.

In another embodiment of the invention there is provided a compound of the formula IB as hereinbefore defined wherein:

X^{a} is O;

one of A¹ and A² is N and the other of A¹ and A² is CH;

R⁴ is hydrogen;

—X—Y-Z- is —(CH₂)₃— or *—O(CH₂)₂—, wherein * indicates the point of attachment of —X—Y-Z- to the ring containing A¹ and A²,

R⁶ is selected from:

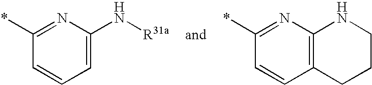

wherein R^{31a} has any of the values defined herein, particularly R^{31a} is methyl; and the group: 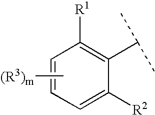
in formula (IB) is as defined in any one of paragraphs (35), (35a), (35b), (36) or (36a) above;
or a pharmaceutically acceptable salt or prodrug thereof.

In another embodiment of the invention there is provided a compound of the formula I which is of the formula IC:

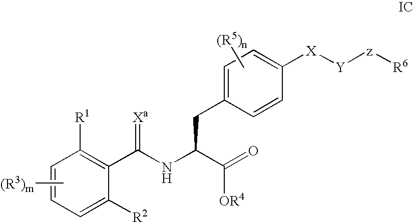

wherein:

n, m, R¹, R², R³, R⁴, R⁵, R⁶, X^{a}, X, Y and Z have any of the values hereinbefore defined;

or a pharmaceutically acceptable salt or prodrug thereof.

In another embodiment of the invention there is provided a compound of the formula I which is of the formula IC as hereinbefore defined wherein:

X^{a} is oxygen;

X, Y and Z have any of the values hereinbefore defined;

R¹ is selected from chloro, methyl and ethyl, for example R¹ is chloro or methyl;

R² is selected from hydrogen, halo and (1-4C)alkyl, for example R² is selected from hydrogen, fluoro, chloro and methyl;

m is 0 or 1 and each R³, which may be the same or different, is selected from halo and (1-4C)alkyl, or two R³ substituents optionally form a (1-3C)alkylenedioxy group, for example each R³, which may be the same or different, is selected from fluoro, chloro, bromo and (1-3C)alkyl;

R⁴ is selected from hydrogen and (1-4C)alkyl, wherein R⁴ is optionally bears on carbon a hydroxy or (1-4C)alkoxy substituent, for example R⁴ is hydrogen;

n is 0, 1 or 2 and each R⁵, which may be the same or different, is selected from halo, hydroxy, amino, (1-4C)alkyl, (1-4C)alkoxy (1-4C)alkylamino and di-[(1-4C)alkyl]amino,

and wherein R⁵ optionally bears on carbon one or more R²⁴ substituents selected from halo, hydroxy, amino, (1-4C)alkoxy, (1-4C)alkylamino and di-[(1-4C)alkyl]amino;

R⁶ is a 5 or 6 membered monocyclic heteroaryl, or a 5,6 or 6,6 bicyclic heteroaryl, which heteroaryl contains at least one nitrogen atom and optionally 1 or more (for example 1, 2 or 3) additional heteroatoms selected from O, S and N, and wherein R⁶ optionally bears on carbon one or more R³¹ substituents selected from amino, (1-4C)alkyl, (1-4C)alkylamino and di-[(1-4C)alkyl]amino,

or from a group of the formula:
—X⁹-Q⁶

wherein X⁹ is a direct bond or is N(R³⁴), wherein R³⁴ is hydrogen or (1-4C)alkyl, and Q⁶ is (3-6C)cycloalkyl or (3-6C)cycloalkyl-(1-4C)alkyl,

and wherein if R⁶ contains an —NH— moiety, the nitrogen of said moiety optionally bears a group selected from (1-4C)alkyl, (for example R⁶ is one of the groups defined in any of (97) to (118) above);

or a pharmaceutically acceptable salt or prodrug thereof.

In the compounds of formula IC a particular value for R⁴ is hydrogen.

Also, in the compounds of formula IC a particular value for X^{a} is oxygen.

In another embodiment of the invention there is provided a compound of the formula I which is of the formula IC as hereinbefore defined wherein:

X^{a} is O or S (particularly O);

R¹ is selected from chloro, bromo, methyl, ethyl, cyclopropyl, cyclobutyl and trifluoromethyl (particularly R¹ is chloro or methyl);

R² is selected from hydrogen, fluoro, chloro, bromo, methyl, ethyl, cyclopropyl, cyclobutyl and trifluoromethyl (particularly R² is selected from hydrogen, fluoro, chloro and methyl, more particularly R² is methyl or fluoro);

m and R³ are as defined in any one of paragraphs (25) to (33) above;

R⁴ is hydrogen;

n is 0, 1 or 2 (suitably n is 0) and each R⁵, which may be the same or different, is as defined in any of (47) to (49), for example each R⁵, is selected from halo, amino, (1-4C)alkyl, (1-4C)alkoxy, (1-4C)alkylamino and di-[(1-4C)alkyl]amino,

and wherein R⁵ optionally bears on carbon one or more R²⁴ substituents selected from halo, hydroxy, amino, (1-4C)alkoxy, (1-4C)alkylamino and di-[(1-4C)alkyl]amino;

the group —X—Y-Z- is as defined in any one of paragraphs (93) to (96) above (for example —X—Y-Z- is —(CH₂)₃— or *—O(CH₂)₂—, wherein * indicates the point of attachment of —X—Y-Z- to the 1,4-phenylene ring in formula (IC));

R⁶ is as defined in any one of paragraphs (97) to (118) (particularly R⁶ is as defined in any one of paragraphs (114) to (118) more particularly wherein R⁶ is attached to the group —X—Y-Z- by a ring carbon atom in R⁶, which carbon atom is located in an aromatic ring in R⁶);

or a pharmaceutically acceptable salt or prodrug thereof.

In a further embodiment in the compounds of formula (IC) defined above, the chain length between the 1,4-phenylene ring and a ring —N═ in R⁶ is 4 or 5, particularly 5 atoms long;

or when Z is NH and R⁶ is attached to Z by a carbon atom that is in an ortho position to a —N═ ring atom in R⁶, then the chain length of the group —X—Y-Z- is 4 or 5 (particularly 5) atoms long.

In another embodiment of the invention there is provided a compound of the formula IC as hereinbefore defined wherein:

X^{a} is O;

R⁴ is hydrogen;

—X—Y-Z- is —(CH₂)₃— or *—O(CH₂)₂—, wherein * indicates the point of attachment of —X—Y-Z- to the phenylene ring;

R⁶ is selected from:

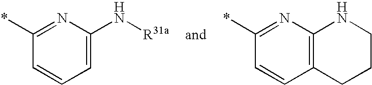

wherein R^{31a} has any of the values defined herein, particularly R^{31a} is methyl; and the group: 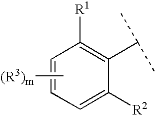
in formula (IB) is as defined in any one of paragraphs (35), (35a), (35b), (36) or (36a) above;
or a pharmaceutically acceptable salt or prodrug thereof.

In another embodiment of the invention there is provided a compound of the formula I which is of the formula ID:

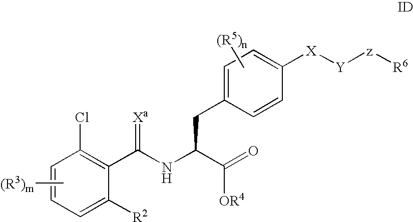

wherein:

R² is selected from hydrogen, fluoro, chloro, methyl and ethyl (for example R² is selected from hydrogen, chloro and methyl); and

n, m, R³, R⁴, R⁵, R⁶, X^{a}, X, Y and Z have any of the values hereinbefore defined;

or a pharmaceutically acceptable salt or prodrug thereof.

In another embodiment of the invention there is provided a compound of the formula I which is of the formula ID as hereinbefore defined wherein:

X^{a} is oxygen;

X, Y and Z are as hereinbefore defined;

R² is selected from hydrogen, chloro and methyl (for example R² is chloro);

m is 0 or 1 and R³ is selected from fluoro, chloro, bromo and (1-3C)alkyl (for example R² is chloro or methyl;

R⁴ is selected from hydrogen and (1-4C)alkyl, wherein R⁴ is optionally bears on carbon a hydroxy or (1-4C)alkoxy substituent, for example R⁴ is hydrogen;

n is 0, 1 or 2 and each R⁵ which may be the same or different, is selected from halo, hydroxy, amino, (1-4C)alkyl, (1-4C)alkoxy (1-4C)alkylamino and di-[(1-4C)alkyl]amino,

R⁶ is a 5 or 6 membered monocyclic heteroaryl, or a 5,6 or 6,6 bicyclic heteroaryl, which heteroaryl contains at least one nitrogen atom and optionally 1 or more (for example 1, 2, 3 or 4) additional heteroatoms selected from O, S and N, and wherein R⁶ optionally bears on carbon one or more R³¹ substituents selected from amino, (1-4C)alkyl, (1-4C)alkylamino and di-[(1-4C)alkyl]amino,

or from a group of the formula:
—X⁹-Q⁶

wherein X⁹ is a direct bond or is N(R³⁴), wherein R³⁴ is hydrogen or (1-4C)alkyl, and Q⁶ is (3-6C)cycloalkyl or (3-6C)cycloalkyl-(1-4C)alkyl,

and wherein if R⁶ contains an —NH— moiety, the nitrogen of said moiety optionally bears a group selected from (1-4C)alkyl, (for example R⁶ is one of the groups defined in any of (97) to (118) above);

or a pharmaceutically acceptable salt or prodrug thereof.

In the compounds of formula ID a particular value for R⁴ is hydrogen.

Also, in the compounds of formula ID a particular value for X^{a} is oxygen.

In another embodiment of the invention there is provided a compound of the formula I which is of the formula ID as hereinbefore defined wherein:

X^{a} is O or S (particularly O);

R² is selected from hydrogen, fluoro, chloro, bromo, methyl, ethyl, cyclopropyl, cyclobutyl and trifluoromethyl (particularly R² is selected from hydrogen, fluoro, chloro and methyl, more particularly R² is methyl or fluoro);

m and R³ are as defined in any one of paragraphs (25) to (33) above;

R⁴ is hydrogen;

n is 0, 1 or 2 (suitably n is 0) and each R⁵, which may be the same or different, is as defined in any of (47) to (49), for example each R⁵, is selected from halo, amino, (1-4C)alkyl, (1-4C)alkoxy, (1-4C)alkylamino and di-[(1-4C)alkyl]amino,

and wherein R⁵ optionally bears on carbon one or more R²⁴ substituents selected from halo, hydroxy, amino, (1-4C)alkoxy, (1-4C)alkylamino and di-[(1-4C)alkyl]amino;

the group —X—Y-Z- is as defined in any one of paragraphs (93) to (96) above (for example —X—Y-Z- is —(CH₂)₃— or *—O(CH₂)₂—, wherein * indicates the point of attachment of —X—Y-Z- to the 1,4-phenylene ring in formula (ID));

R⁶ is as defined in any one of paragraphs (97) to (118) (particularly R⁶ is as defined in any one of paragraphs (114) to (118) more particularly wherein R⁶ is attached to the group —X—Y-Z- by a ring carbon atom in R⁶, which carbon atom is located in an aromatic ring in R⁶);

or a pharmaceutically acceptable salt or prodrug thereof.

In a further embodiment in the compounds of formula (ID) defined above, the chain length between the 1,4-phenylene ring and a ring —N═ in R⁶ is 4 or 5, particularly 5 atoms long;

or when Z is NH and R⁶ is attached to Z by a carbon atom that is in an ortho position to a —N═ ring atom in R⁶, then the chain length of the group —X—Y-Z- is 4 or 5 (particularly 5) atoms long.

In another embodiment of the invention there is provided a compound of the formula I which is of the formula ID as hereinbefore defined wherein:

X^{a} is O;

R⁴ is hydrogen;

—X—Y-Z- is —(CH₂)₃— or *—O(CH₂)₂—, wherein * indicates the point of attachment of —X—Y-Z- to the phenylene ring;

R⁶ is selected from:

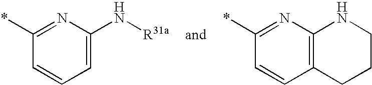

wherein R^{31a} has any of the values defined herein, particularly R^{31a} is methyl; and

R² has any of the values defined above in relation to the compound of formula ID;

or a pharmaceutically acceptable salt or prodrug thereof.

In another embodiment of the invention there is provided a compound of the formula I which is of the formula IE: 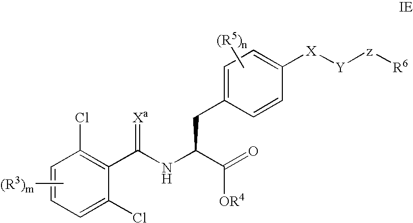
wherein:

X is selected from a direct bond, NR²⁶, O, S, SO, SO₂, C(O), CH(OR²⁶), C(O)N(R²⁶), N(R²⁶)C(O), SO₂N(R²⁶), N(R²⁶)SO₂, CH═CH and C≡C wherein R²⁶ is hydrogen, (1-6C)alkyl or (3-7C)cycloalkyl;

Y is (1-6C)alkylene,

and wherein adjacent carbon atoms in any (2-6C)alkylene chain within a Y substituent are optionally separated by the insertion into the chain of a group selected from O, S, SO, SO₂, N(R²⁷), C(O), CH(OR²⁷), C(O)N(R²⁷), N(R²⁷)C(O), SO₂N(R²⁷), N(R²⁷)SO₂, CH═CH and C≡C wherein R²⁷ is hydrogen, (1-6C)alkyl or (3-7C)cycloalkyl;

Z is selected from a direct bond, NR⁶, O, S, SO, SO₂, C(O), CH(OR²⁶), SO₂N(R²⁶), N(R²⁶)SO₂, CH═CH and C≡C, wherein R²⁶ is hydrogen, (1-6C)alkyl or (3-7C)cycloalkyl;

and wherein any X, Y or Z optionally bears on carbon one or more R²⁸ substituents;

and wherein n, m, R³, R⁴, R⁵, R⁶, R²⁸ and X^{a} have any of the values hereinbefore defined;

or a pharmaceutically acceptable salt or prodrug thereof.

In another embodiment of the invention there is provided a compound of the formula I which is of the formula IE as hereinbefore defined wherein:

n, m, R³, R⁴, R⁵ and R⁶ have any of the values hereinbefore defined;

X^{a} is oxygen;

X is selected from a direct bond, N(R²⁶), O, C(O)N(R²⁶), N(R²⁶)C(O) and C≡C, wherein R²⁶ is hydrogen, (1-6C)alkyl or (3-7C)cycloalkyl;

Y is selected from (1-4C)alkylene, and wherein Y optionally bears on carbon one or more R²⁸ substituents selected from (1-3C)alkyl; and

Z is selected from a direct bond and NR²⁶ wherein R²⁶ is hydrogen, (1-4C)alkyl or (3-6C)cycloalkyl;

or a pharmaceutically acceptable salt or prodrug thereof.

In another embodiment of the invention there is provided a compound of the formula I which is of the formula IE as hereinbefore defined wherein:

n, m, R³, R⁴ and R⁵ have any of the values hereinbefore defined;

X^{a} is oxygen;

X is selected from a direct bond and O;

Y is selected from (1-4C)alkylene, and wherein Y optionally bears on carbon one or more R²⁸ substituents selected from (1-3C)alkyl; and

Z is selected from a direct bond and NR²⁶ wherein R²⁶ is hydrogen, (1-4C)alkyl or (3-6C)cycloalkyl;

R⁶ is a 5 or 6 membered monocyclic heteroaryl, or a 5,6 or 6,6 bicyclic heteroaryl, which heteroaryl contains at least one nitrogen atom and optionally 1 or more (for example 1, 2, 3 or 4) additional heteroatoms selected from O, S and N, and wherein R⁶ optionally bears on carbon one or more R³¹ substituents selected from amino, (1-4C)alkyl, (1-4C)alkylamino and di-[(1-4C)alkyl]amino,

or from a group of the formula:
—X⁹-Q⁶

wherein X⁹ is a direct bond or is N(R³⁴), wherein R³⁴ is hydrogen or (1-4C)alkyl, and Q⁶ is (3-6C)cycloalkyl or (3-6C)cycloalkyl-(1-4C)alkyl,

and wherein if R⁶ contains an —NH— moiety, the nitrogen of said moiety optionally bears a group selected from (1-4C)alkyl, (for example R⁶ is one of the groups defined in any of (97) to (118) above);

or a pharmaceutically acceptable salt or prodrug thereof.

In another embodiment of the invention there is provided a compound of the formula I which is of the formula IE as hereinbefore defined wherein:

n, m, R³, R⁴ and R⁵ have any of the values hereinbefore defined;

X^{a} is oxygen;

X is selected from a direct bond and O;

Y is CH₂CH₂, and wherein Y optionally bears on carbon one or more R²⁸ substituents selected from (1-3C)alkyl; and

Z is selected from a direct bond and NR²⁶ wherein R²⁶ is hydrogen, (1-4C)alkyl or (3-6C)cycloalkyl;

R⁶ is a 5 or 6 membered monocyclic heteroaryl, or a 5,6 or 6,6 bicyclic heteroaryl, which heteroaryl contains at least one nitrogen atom and optionally 1 or more (for example 1, 2, 3 or 4) additional heteroatoms selected from O, S and N, and wherein R⁶ optionally bears on carbon one or more R³¹ substituents selected from amino, (1-4C)alkyl, (1-4C)alkylamino and di-[(1-4C)alkyl]amino,

or from a group of the formula:
—X⁹-Q⁶

wherein X⁹ is a direct bond or is N(R³⁴), wherein R³⁴ is hydrogen or (1-4C)alkyl, and Q⁶ is (3-6C)cycloalkyl or (3-6C)cycloalkyl-(1-4C)alkyl,

and wherein if R⁶ contains an —NH— moiety, the nitrogen of said moiety optionally bears a group selected from (1-4C)alkyl, (for example R⁶ is one of the groups defined in any of (97) to (118) above);

or a pharmaceutically acceptable salt or prodrug thereof.

In this embodiment a particular value for X is O, Y is CH₂CH₂ and Z is a direct bond.

In the compounds of formula IE a particular value for R⁴ is hydrogen.

In the compounds of formula IE a particular value for n is 0, 1 or 2 and each R⁵ which may be the same or different, is selected from halo, hydroxy, amino, (1-4C)alkyl, (1-4C)alkoxy (1-4C)alkylamino and di-[(1-4C)alkyl]amino.

Also in the compounds of formula IE, a particular value for X^{a} is oxygen.

In another embodiment of the invention there is provided a compound of the formula I which is of the formula IE as hereinbefore defined wherein:

X^{a} is O or S (particularly O);

m and R³ are as defined in any one of paragraphs (25) to (33) above;

R⁴ is hydrogen;

n is 0, 1 or 2 (suitably n is 0) and each R⁵, which may be the same or different, is as defined in any of (47) to (49), for example each R⁵, is selected from halo, amino, (1-4C)alkyl, (1-4C)alkoxy, (1-4C)alkylamino and di-[(1-4C)alkyl]amino,

and wherein R⁵ optionally bears on carbon one or more R²⁴ substituents selected from halo, hydroxy, amino, (1-4C)alkoxy, (1-4C)alkylamino and di-[(1-4C)alkyl]amino;

the group —X—Y-Z- is as defined in any one of paragraphs (93) to (96) above (for example —X—Y-Z- is —(CH₂)₃— or *—O(CH₂)₂—, wherein * indicates the point of attachment of —X—Y-Z- to the 1,4-phenylene ring in formula (IE));

R⁶ is as defined in any one of paragraphs (97) to (118) (particularly R⁶ is as defined in any one of paragraphs (114) to (118) more particularly wherein R⁶ is attached to the group —X—Y-Z- by a ring carbon atom in R⁶, which carbon atom is located in an aromatic ring in R⁶);

or a pharmaceutically acceptable salt or prodrug thereof.

In a further embodiment in the compounds of formula (IE) defined above, the chain length between the 1,4-phenylene ring and a ring —N═ in R⁶ is 4 or 5, particularly 5 atoms long;

or when Z is NH and R⁶ is attached to Z by a carbon atom that is in an ortho position to a —N═ ring atom in R⁶, then the chain length of the group —X—Y-Z- is 4 or 5 (particularly 5) atoms long.

In another embodiment of the invention there is provided a compound of the formula I which is of the formula IE as hereinbefore defined wherein:

X^{a} is oxygen;

R⁴ is hydrogen;

—X—Y-Z- is —(CH₂)₃— or *—O(CH₂)₂—, wherein * indicates the point of attachment of —X—Y-Z- to the phenylene ring;

R⁶ is selected from:

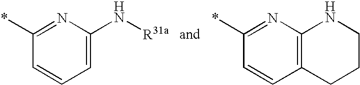

wherein R^{31a} has any of the values defined herein, particularly R^{31a} is methyl;

or a pharmaceutically acceptable salt or prodrug thereof.

In another embodiment of the invention there is provided a compound of the formula I which is of the formula IF:

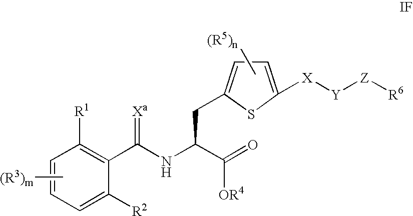

wherein n, m, R¹. R². R³. R⁴. R⁵. R⁶. X, X, Y and Z are as hereinbefore defined;

or a pharmaceutically acceptable salt or prodrug thereof (particularly X is a direct bond, —C═C— or —C≡C—, more particularly X is a direct bond).

In another embodiment of the invention there is provided a compound of the formula I which is of the formula IF as hereinbefore defined wherein:

X^{a} is O or S (particularly O);

R¹ is selected from chloro, bromo, methyl, ethyl, cyclopropyl, cyclobutyl and trifluoromethyl (particularly R¹ is chloro or methyl);

R² is selected from hydrogen, fluoro, chloro, bromo, methyl, ethyl, cyclopropyl, cyclobutyl and trifluoromethyl (particularly R² is selected from hydrogen, fluoro, chloro and methyl, more particularly R² is methyl or fluoro);

m and R³ are as defined in any one of paragraphs (25) to (33) above;

R⁴ is hydrogen;

n is 0, 1 or 2 (suitably n is 0) and each R⁵, which may be the same or different, is as defined in any of (47) to (49), for example each R⁵, is selected from halo, amino, (1-4C)alkyl, (1-4C)alkoxy, (1-4C)alkylamino and di-[(1-4C)alkyl]amino,

and wherein R⁵ optionally bears on carbon one or more R²⁴ substituents selected from halo, hydroxy, amino, (1-4C)alkoxy, (1-4C)alkylamino and di-[(1-4C)alkyl]amino;

the group —X—Y-Z- is as defined in any one of paragraphs (93) to (96) above (for example —X—Y-Z- is —(CH₂)₃—;

R⁶ is as defined in any one of paragraphs (97) to (118) (particularly R⁶ is as defined in any one of paragraphs (114) to (118) more particularly wherein R⁶ is attached to the group —X—Y-Z- by a ring carbon atom in R⁶, which carbon atom is located in an aromatic ring in R⁶);

or a pharmaceutically acceptable salt or prodrug thereof.

In a further embodiment in the compounds of formula (IF) defined above, the chain length between the thiophen-2,5-diyl ring and a ring —N═ in R⁶ is 4 or 5, particularly 5 atoms long;

or when Z is NH and R⁶ is attached to Z by a carbon atom that is in an ortho position to a —N═ ring atom in R⁶, then the chain length of the group —X—Y-Z- is 4 or 5 (particularly 5) atoms long.

In another embodiment of the invention there is provided a compound of the formula IE as hereinbefore defined wherein:

X^{a} is O;

R⁴ is hydrogen;

—X—Y-Z- is —(CH₂)₃—;

R⁶ is selected from:

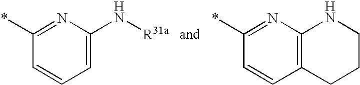

wherein R^{31a} has any of the values defined herein, particularly R^{31a} is methyl; and the group: 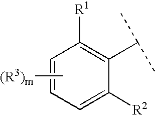
in formula (IB) is as defined in any one of paragraphs (35), (35a), (35b), (36) or (36a) above;
or a pharmaceutically acceptable salt or prodrug thereof.

In another embodiment of the invention there is provided a compound of the formula I which is of the formula IG:

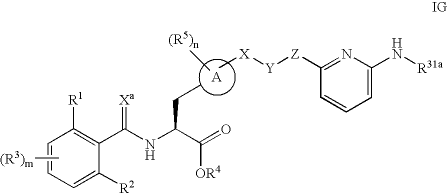

wherein n, m, ring A, R¹, R², R³, R⁴, R⁵, R^{31a}, X^{a}, X, Y and Z are as hereinbefore defined;

or a pharmaceutically acceptable salt or prodrug thereof.

Particular compounds of the formula IG are those wherein ring A is 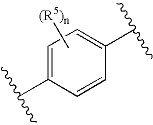
wherein n and R⁵ are as hereinbefore defined.

Other particular compounds of the formula IG are those wherein ring A is 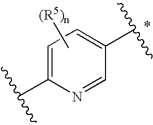
wherein * indicates the point of attachment to the group —X—Y-Z- and n and R⁵, are as hereinbefore defined.

Other particular compounds of the formula IG are those wherein ring A is 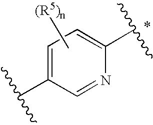
wherein * indicates the point of attachment to the group —X—Y-Z- and n and R⁵, are as hereinbefore defined.

Other particular compounds of the formula IG are those wherein ring A is:

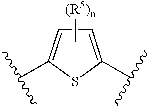

wherein n is 0, 1 or 2 and R⁵ is as hereinbefore defined (and suitably —X is a direct bond).

In another embodiment of the invention there is provided a compound of the formula I which is of the formula IG as hereinbefore defined wherein:

X^{a} is O or S (particularly O);

R¹ is selected from chloro, bromo, methyl, ethyl, cyclopropyl, cyclobutyl and trifluoromethyl (particularly R¹ is chloro or methyl);

R² is selected from hydrogen, fluoro, chloro, bromo, methyl, ethyl, cyclopropyl, cyclobutyl and trifluoromethyl (particularly R² is selected from hydrogen, fluoro, chloro and methyl, more particularly R² is methyl or fluoro);

m and R³ are as defined in any one of paragraphs (25) to (33) above;

R⁴ is hydrogen;

n is 0, 1 or 2 (suitably n is 0) and each R⁵, which may be the same or different, is as defined in any of (47) to (49), for example each R⁵, is selected from halo, amino, (1-4C)alkyl, (1-4C)alkoxy, (1-4C)alkylamino and di-[(1-4C)alkyl]amino,

and wherein R⁵ optionally bears on carbon one or more R²⁴ substituents selected from halo, hydroxy, amino, (1-4C)alkoxy, (1-4C)alkylamino and di-[(1-4C)alkyl]amino;

the group —X—Y-Z- is as defined in any one of paragraphs (93) to (96) above;

R^{31a} is as hereinbefore defined, particularly R^{31a} is selected from hydrogen (1-3C)alkyl, (3-6C)cycloalkyl, or (3-6C)cycloalkyl-(1-3C)alkyl, more particularly R^{31a} is (1-3C)alkyl, cyclopropyl or cyclopropylmethyl, for example R^{31a} is methyl; and

ring A has any of the values as hereinbefore defined in relation to the compound of formula IG (for example ring A is 1,4-phenylene or ring A is thiophen-2,5-di-yl);

or a pharmaceutically acceptable salt or prodrug thereof.

In a particular embodiment in the compounds of formula IG, the group —X—Y-Z- is 3 or 4 (particularly 3) atoms long (for example —X—Y-Z- is —(CH₂)₃— or *—O(CH₂)₂—, wherein * indicates the point of attachment of —X—Y-Z- to the ring A).

In another embodiment of the invention there is provided a compound of the formula I which is of the formula IG as hereinbefore defined wherein:

X^{a} is oxygen;

R⁴ is hydrogen;

—X—Y-Z- is —(CH₂)₃— or *—O(CH₂)₂—, wherein * indicates the point of attachment of —X—Y-Z- to Ring A (for example —X—Y-Z- is —(CH₂)₃—);
Ring A is 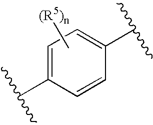
wherein n and R⁵ are as hereinbefore defined (particularly n is 0);
the group: 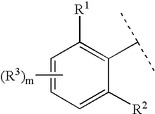
in formula (IG) is as defined in any one of paragraphs (35), (35a), (35b), (36) or (36a) above; and
R^{31a} is as hereinbefore defined in relation to the compound of formula (IG);
or a pharmaceutically acceptable salt or prodrug thereof.

In another embodiment of the invention there is provided a compound of the formula I which is of the formula IG as hereinbefore defined wherein:

X^{a} is oxygen;

R⁴ is hydrogen;

—X—Y-Z- is —(CH₂)₃— or *—O(CH₂)₂—, wherein * indicates the point of attachment of —X—Y-Z- to Ring A;
Ring A is 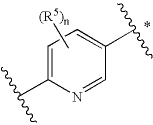
wherein n and R⁵ are as hereinbefore defined (particularly n is 0);
the group: 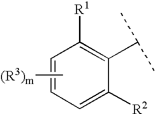
in formula (IG) is as defined in any one of paragraphs (35), (35a), (35b), (36) or (36a) above; and
R^{31a} is as hereinbefore defined in relation to the compound of formula (IG);
or a pharmaceutically acceptable salt or prodrug thereof.

In another embodiment of the invention there is provided a compound of the formula I which is of the formula IG as hereinbefore defined wherein:

X^{a} is oxygen;

R⁴ is hydrogen;

—X—Y-Z- is —(CH₂)₃— or *—O(CH₂)₂—, wherein * indicates the point of attachment of —X—Y-Z- to Ring A;
Ring A is 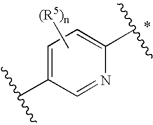
wherein n and R⁵ are as hereinbefore defined (particularly n is 0);
the group: 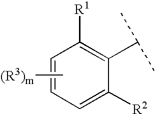
in formula (IG) is as defined in any one of paragraphs (35), (35a), (35b), (36) or (36a) above; and
R^{31a} is as hereinbefore defined in relation to the compound of formula (IG);
or a pharmaceutically acceptable salt or prodrug thereof.

In another embodiment of the invention there is provided a compound of the formula I which is of the formula IG as hereinbefore defined wherein:

X^{a} is oxygen;

R⁴ is hydrogen;

—X—Y-Z- is —(CH₂)₃—;
Ring A is 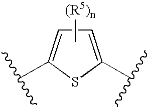
wherein n and R⁵ are as hereinbefore defined (particularly n is 0);
the group: 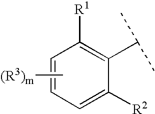
in formula (IG) is as defined in any one of paragraphs (35), (35a), (35b), (36) or (36a) above; and
R^{31a} is as hereinbefore defined in relation to the compound of formula (IG);
or a pharmaceutically acceptable salt or prodrug thereof.

In another embodiment of the invention there is provided a compound of the formula I which is of the formula IH:

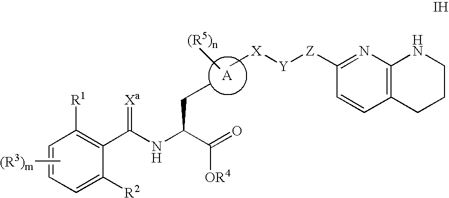

wherein n, m, ring A, R¹, R², R³, R⁴, R⁵, X^{a}, X, Y and Z are as hereinbefore defined;

or a pharmaceutically acceptable salt or prodrug thereof.

Particular compounds of the formula IH are those wherein ring A is 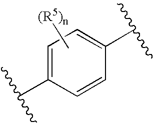
wherein n and R⁵ are as hereinbefore defined.

Other particular compounds of the formula IH are those wherein ring A is 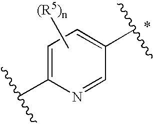
wherein * indicates the point of attachment to the group —X—Y-Z- and n and R⁵, are as hereinbefore defined.

Other particular compounds of the formula IH are those wherein ring A is 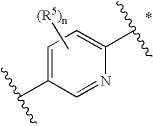
wherein * indicates the point of attachment to the group —X—Y-Z- and n and R⁵, are as hereinbefore defined.

Other particular compounds of the formula IH are those wherein ring A is:

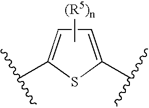

wherein n is 0, 1 or 2 and R⁵ is as hereinbefore defined.

In another embodiment of the invention there is provided a compound of the formula I which is of the formula IH as hereinbefore defined wherein:

X^{a} is O or S (particularly O);

R¹ is selected from chloro, bromo, methyl, ethyl, cyclopropyl, cyclobutyl and trifluoromethyl (particularly R¹ is chloro or methyl);

R² is selected from hydrogen, fluoro, chloro, bromo, methyl, ethyl, cyclopropyl, cyclobutyl and trifluoromethyl (particularly R² is selected from hydrogen, fluoro, chloro and methyl, more particularly R² is methyl or fluoro);

m and R³ are as defined in any one of paragraphs (25) to (33) above;

R⁴ is hydrogen;

n is 0, 1 or 2 (suitably n is 0) and each R⁵, which may be the same or different, is as defined in any of (47) to (49), for example each R⁵, is selected from halo, amino, (1-4C)alkyl, (1-4C)alkoxy, (1-4C)alkylamino and di-[(1-4C)alkyl]amino,

and wherein R⁵ optionally bears on carbon one or more R²⁴ substituents selected from halo, hydroxy, amino, (1-4C)alkoxy, (1-4C)alkylamino and di-[(1-4C)alkyl]amino;

the group —X—Y-Z- is as defined in any one of paragraphs (93) to (96) above;

R^{31a} is as hereinbefore defined, particularly R^{31a} is selected from hydrogen (1-3C)alkyl, (3-6C)cycloalkyl, or (3-6C)cycloalkyl-(1-3C)alkyl, more particularly R^{31a} is (1-3C)alkyl, cyclopropyl or cyclopropylmethyl, for example R^{31a} is methyl; and

ring A has any of the values as hereinbefore defined in relation to the compound of formula IH (for example ring A is 1,4-phenylene or ring A is thiophen-2,5-di-yl);

or a pharmaceutically acceptable salt or prodrug thereof.

In a particular embodiment in the compounds of formula IH, the group —X—Y-Z- is 3 or 4 (particularly 3) atoms long (for example —X—Y-Z- is —(CH₂)₃— or *—O(CH₂)₂—, wherein * indicates the point of attachment of —X—Y-Z- to the ring A).

In another embodiment of the invention there is provided a compound of the formula I which is of the formula IH as hereinbefore defined wherein:

X^{a} is oxygen;

R⁴ is hydrogen;

—X—Y-Z- is —(CH₂)₃— or *—O(CH₂)₂—, wherein * indicates the point of attachment of —X—Y-Z- to Ring A (for example —X—Y-Z- is —(CH₂)₃—);
Ring A is 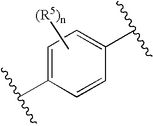
wherein n and R⁵ are as hereinbefore defined (particularly n is 0);
the group: 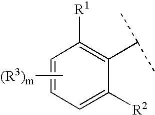
in formula (IG) is as defined in any one of paragraphs (35), (35a), (35b), (36) or (36a) above;
or a pharmaceutically acceptable salt or prodrug thereof.

In another embodiment of the invention there is provided a compound of the formula I which is of the formula IH as hereinbefore defined wherein:

X^{a} is oxygen;

R⁴ is hydrogen;

—X—Y-Z- is —(CH₂)₃— or *—O(CH₂)₂—, wherein * indicates the point of attachment of —X—Y-Z- to Ring A;
Ring A is 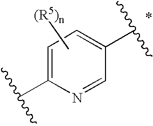
wherein n and R⁵ are as hereinbefore defined (particularly n is 0);
the group: 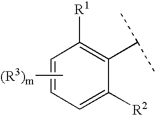
in formula (IH) is as defined in any one of paragraphs (35), (35a), (35b), (36) or (36a) above;
or a pharmaceutically acceptable salt or prodrug thereof.

In another embodiment of the invention there is provided a compound of the formula I which is of the formula IH as hereinbefore defined wherein:

X^{a} is oxygen;

R⁴ is hydrogen;

—X—Y-Z- is —(CH₂)₃— or *—O(CH₂)₂—, wherein * indicates the point of attachment of —X—Y-Z- to Ring A;
Ring A is 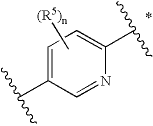
wherein n and R⁵ are as hereinbefore defined (particularly n is 0);
the group: 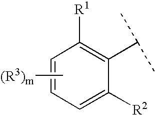
in formula (IG) is as defined in any one of paragraphs (35), (35a), (35b), (36) or (36a) above;
or a pharmaceutically acceptable salt or prodrug thereof.

In another embodiment of the invention there is provided a compound of the formula I which is of the formula IH as hereinbefore defined wherein:

X^{a} is oxygen;

R⁴ is hydrogen;

—X—Y-Z- is —(CH₂)₃—;
Ring A is 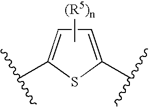
wherein n and R⁵ are as hereinbefore defined (particularly n is 0);
the group: 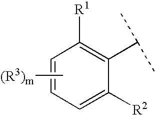
in formula (IH) is as defined in any one of paragraphs (35), (35a), (35b), (36) or (36a) above;
or a pharmaceutically acceptable salt or prodrug thereof.

Accordingly a particular compound of the formula (IH) is a compound of the formula I which is of the formula IH′:

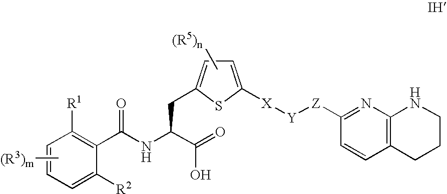

wherein n, m, ring A, R¹, R², R³, R⁵ are as hereinbefore defined; and

—X—Y-Z- is —(CH₂)₃— and wherein —X—Y-Z- optionally bears on carbon 1 or 2 (1-3C)alkyl substituents (for example 1 or 2 methyl or ethyl groups);

or a pharmaceutically acceptable salt or prodrug thereof.

Particular compounds of the formula (IH′) are those wherein the group: 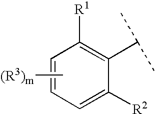
in formula (IH′) is as defined in any one of paragraphs (35), (35a), (35b), (36) or (36a) above.

Other particular compounds of the formula (IH′) are those wherein n=0.

In another embodiment of the invention there is provided a compound of the formula I which is of the formula IJ:

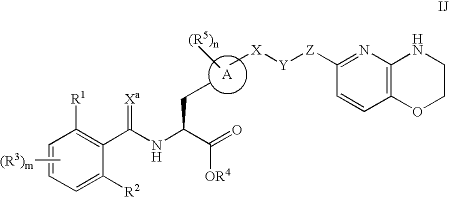

wherein n, m, ring A, R¹, R², R³, R⁴, R⁵, X^{a}, X, Y and Z are as hereinbefore defined;

or a pharmaceutically acceptable salt or prodrug thereof.

Particular compounds of the formula IJ are those wherein ring A is 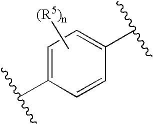
wherein n and R⁵ are as hereinbefore defined.

Other particular compounds of the formula IJ are those wherein ring A is 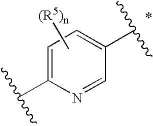
wherein * indicates the point of attachment to the group —X—Y-Z- and n and R⁵, are as hereinbefore defined.

Other particular compounds of the formula IJ are those wherein ring A is 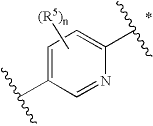
wherein * indicates the point of attachment to the group —X—Y-Z- and n and R⁵, are as hereinbefore defined.

Other particular compounds of the formula IJ are those wherein ring A is:

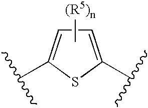

wherein n is 0, 1 or 2 and R⁵ is as hereinbefore defined.

In another embodiment of the invention there is provided a compound of the formula I which is of the formula IJ as hereinbefore defined wherein:

X^{a} is O or S (particularly O);

R¹ is selected from chloro, bromo, methyl, ethyl, cyclopropyl, cyclobutyl and trifluoromethyl (particularly R¹ is chloro or methyl);

R² is selected from hydrogen, fluoro, chloro, bromo, methyl, ethyl, cyclopropyl, cyclobutyl and trifluoromethyl (particularly R² is selected from hydrogen, fluoro, chloro and methyl, more particularly R² is methyl or fluoro);

m and R³ are as defined in any one of paragraphs (25) to (33) above;

R⁴ is hydrogen;

n is 0, 1 or 2 (suitably n is 0) and each R⁵, which may be the same or different, is as defined in any of (47) to (49), for example each R⁵, is selected from halo, amino, (1-4C)alkyl, (1-4C)alkoxy, (1-4C)alkylamino and di-[(1-4C)alkyl]amino,

and wherein R⁵ optionally bears on carbon one or more R²⁴ substituents selected from halo, hydroxy, amino, (1-4C)alkoxy, (1-4C)alkylamino and di-[(1-4C)alkyl]amino;

the group —X—Y-Z- is as defined in any one of paragraphs (93) to (96) above;

R^{31a} is as hereinbefore defined, particularly R^{31a} is selected from hydrogen (1-3C)alkyl, (3-6C)cycloalkyl, or (3-6C)cycloalkyl-(1-3C)alkyl, more particularly R^{31a} is (1-3C)alkyl, cyclopropyl or cyclopropylmethyl, for example R^{31a} is methyl; and

ring A has any of the values as hereinbefore defined in relation to the compound of formula IJ (for example ring A is 1,4-phenylene or ring A is thiophen-2,5-di-yl);

or a pharmaceutically acceptable salt or prodrug thereof.

In a particular embodiment in the compounds of formula IJ, the group —X—Y-Z- is 3 or 4 (particularly 3) atoms long (for example —X—Y-Z- is —(CH₂)₃— or *—O(CH₂)₂—, wherein * indicates the point of attachment of —X—Y-Z- to the ring A).

In another embodiment of the invention there is provided a compound of the formula I which is of the formula IJ as hereinbefore defined wherein:

X^{a} is oxygen;

R⁴ is hydrogen;

—X—Y-Z- is —(CH₂)₃— or *—O(CH₂)₂—, wherein * indicates the point of attachment of —X—Y-Z- to Ring A (for example —X—Y-Z- is —(CH₂)₃—);
Ring A is 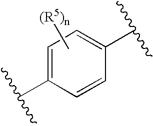
wherein n and R⁵ are as hereinbefore defined (particularly n is 0);
the group: 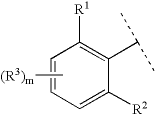
in formula (IJ) is as defined in any one of paragraphs (35), (35a), (35b), (36) or (36a) above;
or a pharmaceutically acceptable salt or prodrug thereof.

In another embodiment of the invention there is provided a compound of the formula I which is of the formula IJ as hereinbefore defined wherein:

X^{a} is oxygen;

R⁴ is hydrogen;

—X—Y-Z- is —(CH₂)₃— or *—O(CH₂)₂—, wherein * indicates the point of attachment of —X—Y-Z- to Ring A;
Ring A is 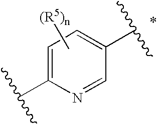
wherein n and R⁵ are as hereinbefore defined (particularly n is 0);
the group: 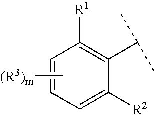
in formula (IJ) is as defined in any one of paragraphs (35), (35a), (35b), (36) or (36a) above;
or a pharmaceutically acceptable salt or prodrug thereof.

In another embodiment of the invention there is provided a compound of the formula I which is of the formula IJ as hereinbefore defined wherein:

X^{a} is oxygen;

R⁴ is hydrogen;

—X—Y-Z- is —(CH₂)₃— or *—O(CH₂)₂—, wherein * indicates the point of attachment of —X—Y-Z- to Ring A;
Ring A is 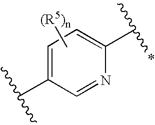
wherein n and R⁵ are as hereinbefore defined (particularly n is 0);
the group: 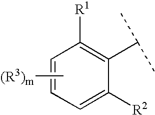
in formula (IJ) is as defined in any one of paragraphs (35), (35a), (35b), (36) or (36a) above;
or a pharmaceutically acceptable salt or prodrug thereof.

In another embodiment of the invention there is provided a compound of the formula I which is of the formula IJ as hereinbefore defined wherein:

X^{a} is oxygen;

R⁴ is hydrogen;

—X—Y-Z- is —(CH₂)₃—;
Ring A is 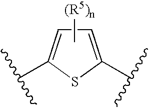
wherein n and R⁵ are as hereinbefore defined (particularly n is 0);
the group: 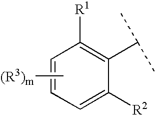
in formula (IJ) is as defined in any one of paragraphs (35), (35a), (35b), (36) or (36a) above;
or a pharmaceutically acceptable salt or prodrug thereof.

In another embodiment of the invention there is provided a compound of the formula I selected from:
N-(2,6-dichlorobenzoyl)-O-[2-(pyridin-2-ylamino)ethyl]-L-tyrosine;
N-(2,6-dichlorobenzoyl)-O-[3-(pyridin-2-ylamino)propyl]-L-tyrosine;
N-(2,6-dichlorobenzoyl)-O-[4-(pyridin-2-ylamino)butyl]-L-tyrosine;
N-(2,6-dichlorobenzoyl)-O-(5,6,7,8-tetrahydro-1,8-naphthyridin-2-ylmethyl)-L-tyrosine;
N-(2,6-dichlorobenzoyl)-O-[2-(5,6,7,8-tetrahydro-1,8-naphthyridin-2-yl)ethyl]-L-tyrosine;
N-(2,6-dichlorobenzoyl)-O-{[6-(methylamino)pyridin-2-yl]methyl}-L-tyrosine;
N-(2,6-dichlorobenzoyl)-O-{2-[6-(methylamino)pyridin-2-yl]ethyl}-L-tyrosine;
N-(2,6-dichlorobenzoyl)-O-{2-[6-(methylamino)pyridin-2-yl]butyl}-L-tyrosine;
N-(2,6-dichlorobenzoyl)-O-{3-[6-(methylamino)pyridin-2-yl]propyl}-L-tyrosine;
N-(2,6-dichlorobenzoyl)-4-[3-(pyridin-2-ylamino)prop-1-yn-1-yl]-L-phenylalanine;
N-(2,6-dichlorobenzoyl)-4-[3-(pyridin-2-ylamino)propyl]-L-phenylalanine;
N-(2,6-dichlorobenzoyl)-4-[5-(pyridin-2-ylamino)pent-1-yn-1-yl]-L-phenylalanine;
O-[2-(1H-benzimidazol-2-ylamino)ethyl]-N-(2,6-dichlorobenzoyl)-L-tyrosine;
N-(2-chlorobenzoyl)-O-[3-(pyridin-2-ylamino)propyl]-L-tyrosine;
N-(2,4-dichlorobenzoyl)-O-[3-(pyridin-2-ylamino)propyl]-L-tyrosine;
N-(2-chloro-5-methylbenzoyl)-O-[3-(pyridin-2-ylamino)propyl]-L-tyrosine;
N-(2-chloro-6-fluorobenzoyl)-O-[3-(pyridin-2-ylamino)propyl]-L-tyrosine;
N-(2-chloro-6-fluoro-3-methylbenzoyl)-O-[3-(pyridin-2-ylamino)propyl]-L-tyrosine;
N-(6-chloro-2-fluoro-3-methylbenzoyl)-O-[3-(pyridin-2-ylamino)propyl]-L-tyrosine;
N-(3-chloro-2-methylbenzoyl)-O-[3-(pyridin-2-ylamino)propyl]-L-tyrosine;
N-(2-chloro-3,6-difluorobenzoyl)-O-[3-(pyridin-2-ylamino)propyl]-L-tyrosine;
N-(2-methylbenzoyl)-O-[3-(pyridin-2-ylamino)propyl]-L-tyrosine;
N-(mesitylcarbonyl)-O-[3-(pyridin-2-ylamino)propyl]-L-tyrosine;
N-(2,6-dichloro-3-methylbenzoyl)-O-[3-(pyridin-2-ylamino)propyl]-L-tyrosine;
N-(2,6-dichlorobenzoyl)-4-[4-(pyridin-2-ylamino)but-1-yn-1-yl]-L-phenylalanine;
N-(2,6-dichlorobenzoyl)-3-{5-[2-(5,6,7,8-tetrahydro-1,8-naphthyridin-2-yl)ethoxy]pyridin-2-yl}-L-alanine; and
N-(2,6-dimethylbenzoyl)-O-[3-(pyridin-2-ylamino)propyl]-L-tyrosine;
N-[(5-chloro-1,3-benzodioxol-4-yl)carbonyl]-O-[3-(pyridin-2-ylamino)propyl]-L-tyrosine;
N-(2-chloro-4-fluorobenzoyl)-O-[3-(pyridin-2-ylamino)propyl]-L-tyrosine;
N-(2-bromo-6-chlorobenzoyl)-O-[3-(pyridin-2-ylamino)propyl]-L-tyrosine;
N-(2,6-dichlorobenzoyl)-3-[2-(pyridin-2-ylamino)ethoxy]-L-phenylalanine;
N-(2,6-dichlorobenzoyl)-3-[3-(pyridin-2-ylamino)propoxy]-L-phenylalanine;
N-(2,6-dichlorobenzoyl)-3-[4-(pyridin-2-ylamino)butoxy]-L-phenylalanine;
N-(2,6-dichlorobenzoyl)-O-[2-(1-methyl-1,2,3,4-tetrahydropyrido[2,3-b]pyrazin-6-yl)ethyl]-L-tyrosine;
N-(2,6-dichlorobenzoyl)-4-{3-[6-(methylamino)pyridin-2-yl]propyl}-L-phenylalanine;
N-(2,6-dichlorobenzoyl)-4-{3-[6-(methylamino)pyridin-2-yl]propyl}-L-phenylalanine;
N-(2-chlorobenzoyl)-4-{3-[6-(methylamino)pyridin-2-yl]propyl}-L-phenylalanine;
N-(2-chloro-6-fluorobenzoyl)-4-{3-[6-(methylamino)pyridin-2-yl]propyl}-L-phenylalanine;
N-(2-chloro-4-fluorobenzoyl)-4-{3-[6-(methylamino)pyridin-2-yl]propyl}-L-phenylalanine;
N-(2-chloro-6-fluoro-3-methylbenzoyl)-4-{3-[6-(methylamino)pyridin-2-yl]propyl}-L-phenylalanine;
N-(2,6-dichlorobenzoyl)-3-fluoro-O-{2-[6-(methylamino)pyridin-2-yl]ethyl}-L-tyrosine;
N-(2,6-dichlorobenzoyl)-3-methyl-O-{2-[6-(methylamino)pyridin-2-yl]ethyl}-L-tyrosine;
N-(2,6-dichlorobenzoyl)-3,5-dimethyl-O-{2-[6-(methylamino)pyridin-2-yl]ethyl}-L-tyrosine;
N-(2,6-dichlorobenzoyl)-O-{3-[(5-methylpyridin-2-yl)amino]propyl}-L-tyrosine;
N-(2,6-dichlorobenzoyl)-O-{3-[(4-methylpyridin-2-yl)amino]propyl}-L-tyrosine;
N-(2,6-dichlorobenzoyl)-O-{3-[(6-methylpyridin-2-yl)amino]propyl}-L-tyrosine;
N-(2,6-dichlorobenzoyl)-O-{3-[(3-methylpyridin-2-yl)amino]propyl}-L-tyrosine;
N-(2,6-dichlorobenzoyl)-O-{3-[(4,6-dimethylpyridin-2-yl)amino]propyl}-L-tyrosine;
N-(2,6-dichlorobenzoyl)-O-{3-[(4-ethylpyridin-2-yl)amino]propyl}-L-tyrosine;
N-(2,6-dichlorobenzoyl)-4-[4-(pyridin-2-ylamino)butyl]-L-phenylalanine;
N-(2,6-dichlorobenzoyl)-O-[2-(5,6,7,8-tetrahydro-1,8-naphthyridin-2-yl)propyl]-L-tyrosine;
N-(2,6-dichlorobenzoyl)-O-[2-methyl-2-(5,6,7,8-tetrahydro-1,8-naphthyridin-2-yl)propyl]-L-tyrosine;
N-(2-chloro-6-fluorobenzoyl)-O-[2-(3,4-dihydro-2H-pyrido[3,2-b][1,4]oxazin-6-yl)ethyl]-L-tyrosine;
N-(2-chlorobenzoyl)-O-[2-(3,4-dihydro-2H-pyrido[3,2-b][1,4]oxazin-6-yl)ethyl]-L-tyrosine;
N-(2-chloro-4-fluorobenzoyl)-O-[2-(3,4-dihydro-2H-pyrido[3,2-b][1,4]oxazin-6-yl)ethyl]-L-tyrosine;
N-(2-chloro-6-fluoro-3-methylbenzoyl)-O-[2-(3,4-dihydro-2H-pyrido[3,2-b][1,4]oxazin-6-yl)ethyl]-L-tyrosine;
N-(2-chloro-6-fluorobenzoyl)-O-[2-(3,4-dihydro-2H-pyrido[3,2-b][1,4]oxazin-6-yl)ethyl]-L-tyrosine; and
N-(2,6-dichlorobenzoyl)-3-(5-{3-[6-(methylamino)pyridin-2-yl]propyl}-2-thienyl)-L-alanine;
   or a pharmaceutically acceptable salt or prodrug thereof.

In another embodiment of the invention there is provided a compound of the formula I selected from:
N-[(2,6-dichlorophenyl)carbonyl]-4-({[2-(pyridin-2-ylamino)ethyl]amino}methyl)-L-phenylalanine;
N-[(2,6-dichlorophenyl)carbonyl]-O-{3-[(5-methoxypyridin-2-yl)amino]propyl}-L-tyrosine;
N-[(2,6-dichlorophenyl)carbonyl]-O-{3-[(4-methoxypyridin-2-yl)amino]propyl}-L-tyrosine;
N-[(2,6-dichlorophenyl)carbonyl]-O-{2-[5-methoxy-6-(methylamino)pyridin-2-yl]ethyl}-L-tyrosine;
N-[(2,6-dichlorophenyl)carbonyl]-O-[2-(4-methyl-5,6,7,8-tetrahydro-1,8-naphthyridin-2-yl)ethyl]-L-tyrosine;
N-[(2,6-dichlorophenyl)carbonyl]-O-[2-(7-methyl-5,6,7,8-tetrahydro-1,8-naphthyridin-2-yl)ethyl]-L-tyrosine;
N-[(2-chlorophenyl)carbonyl]-4-[3-(3,4-dihydro-2H-pyrido[3,2-b][1,4]oxazin-6-yl)propyl]-L-phenylalanine;
N-[(2-chlorophenyl)carbonyl]-4-[3-(3,4-dihydro-2H-pyrido[3,2-b][1,4]oxazin-6-yl)propyl]-L-phenylalanine;
N-[(2-chloro-6-fluorophenyl)carbonyl]-4-[3-(3,4-dihydro-2H-pyrido[3,2-b][1,4]oxazin-6-yl)propyl]-L-phenylalanine;
N-[(2-chloro-4-fluorophenyl)carbonyl]-4-[3-(3,4-dihydro-2H-pyrido[3,2-b][1,4]oxazin-6-yl)propyl]-L-phenylalanine;
N-[(2,6-dichlorophenyl)carbonyl]-4-[3-(3,4-dihydro-2H-pyrido[3,2-b][1,4]oxazin-6-yl)propyl]-L-phenylalanine;
N-[(2,6-dichlorophenyl)carbonyl]-4-({[6-(methylamino)pyridin-2-yl]methoxy}methyl)-L-phenylalanine;
N-[(2,6-dichlorophenyl)carbonyl]-4-{[2-(methylamino)quinolin-7-yl]methyl}-L-phenylalanine;
N-[(2,4-dichlorophenyl)carbonyl]-O-{2-[6-(methylamino)pyridin-2-yl]ethyl}-L-tyrosine;
N-[(2-chloro-3,6-difluorophenyl)carbonyl]-O-{2-[6-(methylamino)pyridin-2-yl]ethyl}-L-tyrosine;
N-[(2-chloro-5-methylphenyl)carbonyl]-O-{2-[6-(methylamino)pyridin-2-yl]ethyl}-L-tyrosine;
O-{2-[6-(methylamino)pyridin-2-yl]ethyl}-N-[(2,4,6-trimethylphenyl)carbonyl]-L-tyrosine;
N-[(2-chloro-6-methylphenyl)carbonyl]-O-{2-[6-(methylamino)pyridin-2-yl]ethyl}-L-tyrosine;
N-[(2-chloro-4-fluorophenyl)carbonyl]-O-{2-[6-(methylamino)pyridin-2-yl]ethyl}-L-tyrosine;
N-[(2-chloro-6-fluorophenyl)carbonyl]-O-{2-[6-(methylamino)pyridin-2-yl]ethyl}-L-tyrosine;
N-[(2,6-dimethylphenyl)carbonyl]-O-{2-[6-(methylamino)pyridin-2-yl]ethyl}-L-tyrosine;
N-[(6-chloro-2-fluoro-3-methylphenyl)carbonyl]-O-{2-[6-(methylamino)pyridin-2-yl]ethyl}-L-tyrosine;
O-{2-[6-(methylamino)pyridin-2-yl]ethyl}-N-[(2-methylphenyl)carbonyl]-L-tyrosine;
N-[(2-chlorophenyl)carbonyl]-O-{2-[6-(methylamino)pyridin-2-yl]ethyl}-L-tyrosine;
N-[(5-chloro-1,3-benzodioxol-4-yl)carbonyl]-O-{2-[6-(methylamino)pyridin-2-yl]ethyl}-L-tyrosine;
N-[(2-fluoro-6-methylphenyl)carbonyl]-O-{2-[6-(methylamino)pyridin-2-yl]ethyl}-L-tyrosine;
N-[(2,3-dichlorophenyl)carbonyl]-O-{2-[6-(methylamino)pyridin-2-yl]ethyl}-L-tyrosine;
N-{[5-fluoro-2-(trifluoromethyl)phenyl]carbonyl}-O-{2-[6-(methylamino)pyridin-2-yl]ethyl}-L-tyrosine;
O-{2-[6-(methylamino)pyridin-2-yl]ethyl}-N-{[2-(trifluoromethyl)phenyl]carbonyl}-L-tyrosine;
N-[(2,5-dimethylphenyl)carbonyl]-O-{2-[6-(methylamino)pyridin-2-yl]ethyl}-L-tyrosine;
N-[(2-ethylphenyl)carbonyl]-O-{2-[6-(methylamino)pyridin-2-yl]ethyl}-L-tyrosine;
N-[(3-chloro-2-methylphenyl)carbonyl]-O-{2-[6-(methylamino)pyridin-2-yl]ethyl}-L-tyrosine;
N-{[4-(acetylamino)-2-methylphenyl]carbonyl}-O-{2-[6-(methylamino)pyridin-2-yl]ethyl}-L-tyrosine;
N-{[2-fluoro-6-(trifluoromethyl)phenyl]carbonyl}-O-{2-[6-(methylamino)pyridin-2-yl]ethyl}-L-tyrosine;
N-[(2-chloro-6-fluoro-3-methylphenyl)carbonyl]-O-{2-[6-(methylamino)pyridin-2-yl]ethyl}-L-tyrosine;
N-[(4-chloro-2-methylphenyl)carbonyl]-O-{2-[6-(methylamino)pyridin-2-yl]ethyl}-L-tyrosine;
N-{[2-(acetylamino)-6-chlorophenyl]carbonyl}-O-{2-[6-(methylamino)pyridin-2-yl]ethyl}-L-tyrosine;
N-[(2,3-dimethylphenyl)carbonyl]-O-{2-[6-(methylamino)pyridin-2-yl]ethyl}-L-tyrosine;
N-[(2,4-dimethylphenyl)carbonyl]-O-{2-[6-(methylamino)pyridin-2-yl]ethyl}-L-tyrosine;
N-[(4-methoxy-2-methylphenyl)carbonyl]-O-{2-[6-(methylamino)pyridin-2-yl]ethyl}-L-tyrosine;
N-{[4-fluoro-2-(trifluoromethyl)phenyl]carbonyl}-O-{2-[6-(methylamino)pyridin-2-yl]ethyl}-L-tyrosine;
N-[(5-fluoro-2-methylphenyl)carbonyl]-O-{2-[6-(methylamino)pyridin-2-yl]ethyl}-L-tyrosine;
N-[(3,6-dichloro-2-methoxyphenyl)carbonyl]-O-{2-[6-(methylamino)pyridin-2-yl]ethyl}-L-tyrosine;
N-{[2-chloro-5-(trifluoromethyl)phenyl]carbonyl}-O-{2-[6-(methylamino)pyridin-2-yl]ethyl}-L-tyrosine;
N-[(3-fluoro-2-methylphenyl)carbonyl]-O-{2-[6-(methylamino)pyridin-2-yl]ethyl}-L-tyrosine;
N-[(2,4-dichloro-5-fluorophenyl)carbonyl]-O-{2-[6-(methylamino)pyridin-2-yl]ethyl}-L-tyrosine;
O-{2-[6-(methylamino)pyridin-2-yl]ethyl}-N-[(2,4,5-trimethylphenyl)carbonyl]-L-tyrosine;
N-[(2-chloro-4,5-difluorophenyl)carbonyl]-O-{2-[6-(methylamino)pyridin-2-yl]ethyl}-L-tyrosine;
N-[(2-chloro-3,4-dimethoxyphenyl)carbonyl]-O-{2-[6-(methylamino)pyridin-2-yl]ethyl}-L-tyrosine;
N-{[2-chloro-4-(methylsulfonyl)phenyl]carbonyl}-O-{2-[6-(methylamino)pyridin-2-yl]ethyl}-L-tyrosine;
N-[(3-hydroxy-2-methylphenyl)carbonyl]-O-{2-[6-(methylamino)pyridin-2-yl]ethyl}-L-tyrosine;
N-{[2-chloro-3-(trifluoromethyl)phenyl]carbonyl}-O-{2-[6-(methylamino)pyridin-2-yl]ethyl}-L-tyrosine;
N-{[3-chloro-2-fluoro-6-(trifluoromethyl)phenyl]carbonyl}-O-{2-[6-(methylamino)pyridin-2-yl]ethyl}-L-tyrosine;
N-{[5-chloro-2-(trifluoromethyl)phenyl]carbonyl}-O-{2-[6-(methylamino)pyridin-2-yl]ethyl}-L-tyrosine;
N-[(3-methoxy-2-methylphenyl)carbonyl]-O-{2-[6-(methylamino)pyridin-2-yl]ethyl}-L-tyrosine;
N-[(4-fluoro-2-methylphenyl)carbonyl]-O-{2-[6-(methylamino)pyridin-2-yl]ethyl}-L-tyrosine;
N-[(2-chloro-4-fluoro-5-sulfamoylphenyl)carbonyl]-O-{2-[6-(methylamino)pyridin-2-yl]ethyl}-L-tyrosine;
N-[(2-chloro-3-methylphenyl)carbonyl]-O-{2-[6-(methylamino)pyridin-2-yl]ethyl}-L-tyrosine;
N-[(2-chloro-4-methoxyphenyl)carbonyl]-O-{2-[6-(methylamino)pyridin-2-yl]ethyl}-L-tyrosine;
N-[(2-chloro-4-pyrrolidin-1-ylphenyl)carbonyl]-O-{2-[6-(methylamino)pyridin-2-yl]ethyl}-L-tyrosine;
N-[(2-chloro-4-morpholin-4-ylphenyl)carbonyl]-O-{2-[6-(methylamino)pyridin-2-yl]ethyl}-L-tyrosine;
N-{[2-chloro-4-(1H-pyrazol-1-yl)phenyl]carbonyl}-O-{2-[6-(methylamino)pyridin-2-yl]ethyl}-L-tyrosine;
N-[(2-chloro-5-methoxyphenyl)carbonyl]-O-{2-[6-(methylamino)pyridin-2-yl]ethyl}-L-tyrosine;
N-[(2-chloro-4,5-dimethoxyphenyl)carbonyl]-O-{2-[6-(methylamino)pyridin-2-yl]ethyl}-L-tyrosine;
O-{2-[6-(methylamino)pyridin-2-yl]ethyl}-N-{[2-methyl-3-(trifluoromethyl)phenyl]carbonyl}-L-tyrosine;
N-[(5-chloro-2-methylphenyl)carbonyl]-O-{2-[6-(methylamino)pyridin-2-yl]ethyl}-L-tyrosine;
O-{2-[6-(methylamino)pyridin-2-yl]ethyl}-N-{[2-methyl-5-(trifluoromethyl)phenyl]carbonyl}-L-tyrosine;
N-{[3-fluoro-2-(trifluoromethyl)phenyl]carbonyl}-O-{2-[6-(methylamino)pyridin-2-yl]ethyl}-L-tyrosine;
N-{[4-methoxy-2-(trifluoromethyl)phenyl]carbonyl}-O-{2-[6-(methylamino)pyridin-2-yl]ethyl}-L-tyrosine;
N-[(2-chloro-3-fluorophenyl)carbonyl]-O-{2-[6-(methylamino)pyridin-2-yl]ethyl}-L-tyrosine;
N-[(2-chloro-5-fluorophenyl)carbonyl]-O-{2-[6-(methylamino)pyridin-2-yl]ethyl}-L-tyrosine;
N-[(2-ethyl-4-fluorophenyl)carbonyl]-O-{2-[6-(methylamino)pyridin-2-yl]ethyl}-L-tyrosine;
N-[(4,5-difluoro-2-methylphenyl)carbonyl]-O-{2-[6-(methylamino)pyridin-2-yl]ethyl}-L-tyrosine;
N-[(2-ethyl-5-fluorophenyl)carbonyl]-O-{2-[6-(methylamino)pyridin-2-yl]ethyl}-L-tyrosine;
N-[(2-cyclopropyl-4-fluorophenyl)carbonyl]-O-{2-[6-(methylamino)pyridin-2-yl]ethyl}-L-tyrosine;
N-[(2,4-dichlorophenyl)carbonyl]-O-[2-(5,6,7,8-tetrahydro-1,8-naphthyridin-2-yl)ethyl]-L-tyrosine;
N-[(2-chloro-3,6-difluorophenyl)carbonyl]-O-[2-(5,6,7,8-tetrahydro-1,8-naphthyridin-2-yl)ethyl]-L-tyrosine;
N-[(2-chloro-5-methylphenyl)carbonyl]-O-[2-(5,6,7,8-tetrahydro-1,8-naphthyridin-2-yl)ethyl]-L-tyrosine;
O-[2-(5,6,7,8-tetrahydro-1,8-naphthyridin-2-yl)ethyl]-N-[(2,4,6-trimethylphenyl)carbonyl]-L-tyrosine;
N-[(2-chloro-6-methylphenyl)carbonyl]-O-[2-(5,6,7,8-tetrahydro-1,8-naphthyridin-2-yl)ethyl]-L-tyrosine;
N-[(2-chloro-4-fluorophenyl)carbonyl]-O-[2-(5,6,7,8-tetrahydro-1,8-naphthyridin-2-yl)ethyl]-L-tyrosine;
N-[(2-chloro-6-fluorophenyl)carbonyl]-O-[2-(5,6,7,8-tetrahydro-1,8-naphthyridin-2-yl)ethyl]-L-tyrosine;
N-[(2,6-dimethylphenyl)carbonyl]-O-[2-(5,6,7,8-tetrahydro-1,8-naphthyridin-2-yl)ethyl]-L-tyrosine;
N-[(6-chloro-2-fluoro-3-methylphenyl)carbonyl]-O-[2-(5,6,7,8-tetrahydro-1,8-naphthyridin-2-yl)ethyl]-L-tyrosine;
N-[(2-chlorophenyl)carbonyl]-O-[2-(5,6,7,8-tetrahydro-1,8-naphthyridin-2-yl)ethyl]-L-tyrosine;
N-[(5-chloro-1,3-benzodioxol-4-yl)carbonyl]-O-[2-(5,6,7,8-tetrahydro-1,8-naphthyridin-2-yl)ethyl]-L-tyrosine;
N-[(2-methylphenyl)carbonyl]-O-[2-(5,6,7,8-tetrahydro-1,8-naphthyridin-2-yl)ethyl]-L-tyrosine;
N-[(2-fluoro-6-methylphenyl)carbonyl]-O-[2-(5,6,7,8-tetrahydro-1,8-naphthyridin-2-yl)ethyl]-L-tyrosine;
N-[(2-chlorophenyl)carbonyl]-3-(5-{3-[6-(methylamino)pyridin-2-yl]propyl}pyridin-2-yl)-L-alanine;
N-[(2,6-dichlorophenyl)carbonyl]-3-(5-{3-[6-(methylamino)pyridin-2-yl]propyl}pyridin-2-yl)-L-alanine;
N-[(2-chlorophenyl)carbonyl]-4-{4-[6-(methylamino)pyridin-2-yl]butyl}-L-phenylalanine;
N-[(2,6-dichlorophenyl)carbonyl]-4-{4-[6-(methylamino)pyridin-2-yl]butyl}-L-phenylalanine;
N-[(2,6-dichlorophenyl)carbonyl]-O-[2-(5-methyl-5,6,7,8-tetrahydro-1,8-naphthyridin-2-yl)ethyl]-L-tyrosine;
N-[(2-chlorophenyl)carbonyl]-4-[3-(5,6,7,8-tetrahydro-1,8-naphthyridin-2-yl)propyl]-L-phenylalanine;
N-[(2,6-dichlorophenyl)carbonyl]-O-{2-[6-(dimethylamino)pyridin-2-yl]ethyl}-L-tyrosine;
O-{2-[6-(cyclopentylamino)pyridin-2-yl]ethyl}-N-[(2,6-dichlorophenyl)carbonyl]-L-tyrosine;
O-{2-[6-(cyclopropylamino)pyridin-2-yl]ethyl}-N-[(2,6-dichlorophenyl)carbonyl]-L-tyrosine;
O-[2-(6-aminopyridin-2-yl)ethyl]-N-[(2,6-dichlorophenyl)carbonyl]-L-tyrosine;
O-(2-{6-[(cyclopropylmethyl)amino]pyridin-2-yl}ethyl)-N-[(2,6-dichlorophenyl)carbonyl]-L-tyrosine;
N-[(2,6-dichlorophenyl)carbonyl]-O-(2-{6-[(2-methoxyethyl)amino]pyridin-2-yl}ethyl)-L-tyrosine; and
N-[(2,6-dichlorophenyl)carbonyl]-O-[2-(6-{[2-(dimethylamino)ethyl]amino}pyridin-2-yl)ethyl]-L-tyrosine;
N-[(2-bromo-4-fluorophenyl)carbonyl]-O-{2-[6-(methylamino)pyridin-2-yl]ethyl}-L-tyrosine;
N-[(2-bromo-4,5-difluorophenyl)carbonyl]-O-{2-[6-(methylamino)pyridin-2-yl]ethyl}-L-tyrosine;
N-[(2-bromo-5-fluorophenyl)carbonyl]-O-{2-[6-(methylamino)pyridin-2-yl]ethyl}-L-tyrosine;
N-[(2,6-dichlorophenyl)carbonyl]-3-(6-{2-[6-(methylamino)pyridin-2-yl]ethoxy}pyridin-3-yl)-L-alanine;
N-[(2-chloro-4-fluorophenyl)carbonyl]-3-(6-{2-[6-(methylamino)pyridin-2-yl]ethoxy}pyridin-3-yl)-L-alanine;
N-[(2-chloro-4-methoxyphenyl)carbonyl]-3-(6-{2-[6-(methylamino)pyridin-2-yl]ethoxy}pyridin-3-yl)-L-alanine;
N-[(2-ethyl-4-fluorophenyl)carbonyl]-3-(6-{2-[6-(methylamino)pyridin-2-yl]ethoxy}pyridin-3-yl)-L-alanine;
N-[(2,6-dichlorophenyl)carbonyl]-3-(6-{3-[6-(methylamino)pyridin-2-yl]propyl}pyridin-3-yl)-L-alanine;
N-[(2,6-dichlorophenyl)carbonyl]-3-(4-{3-[6-(methylamino)pyridin-2-yl]propyl}thiophen-2-yl)-L-alanine;
N-[(2,6-dichlorophenyl)carbonyl]-3-(4-{2-[6-(methylamino)pyridin-2-yl]ethoxy}thiophen-2-yl)-L-alanine;
N-[(2,6-dimethylphenyl)carbonyl]-3-(5-{3-[6-(methylamino)pyridin-2-yl]propyl}thiophen-2-yl)-L-alanine;
N-[(2-chloro-6-fluorophenyl)carbonyl]-3-(5-{3-[6-(methylamino)pyridin-2-yl]propyl}thiophen-2-yl)-L-alanine;
O-[2-(3,4-dihydro-2H-pyrido[3,2-b][1,4]oxazin-6-yl)ethyl]-N-[(2,6-dimethylphenyl)carbonyl]-L-tyrosine;
N-[(2-chloro-6-methylphenyl)carbonyl]-O-[2-(3,4-dihydro-2H-pyrido[3,2-b][1,4]oxazin-6-yl)ethyl]-L-tyrosine;
O-[2-(3,4-dihydro-2H-pyrido[3,2-b][1,4]oxazin-6-yl)ethyl]-N-[(2-ethyl-4-fluorophenyl)carbonyl]-L-tyrosine;
O-[2-(3,4-dihydro-2H-pyrido[3,2-b][1,4]oxazin-6-yl)ethyl]-N-[(2,4,6-trimethylphenyl)carbonyl]-L-tyrosine;
N-[(2-chloro-4-methoxyphenyl)carbonyl]-O-[2-(3,4-dihydro-2H-pyrido[3,2-b][1,4]oxazin-6-yl)ethyl]-L-tyrosine;
N-[(2-chloro-5-fluorophenyl)carbonyl]-O-[2-(3,4-dihydro-2H-pyrido[3,2-b][1,4]oxazin-6-yl)ethyl]-L-tyrosine;
O-[2-(3,4-dihydro-2H-pyrido[3,2-b][1,4]oxazin-6-yl)ethyl]-N-[(2-ethyl-5-fluorophenyl)carbonyl]-L-tyrosine;
N-[(2-chloro-6-fluorophenyl)carbonyl]-3-(5-{3-[6-(methylamino)pyridin-2-yl]propyl}pyridin-2-yl)-L-alanine;
N-[(2-chloro-4-fluorophenyl)carbonyl]-3-(5-{3-[6-(methylamino)pyridin-2-yl]propyl}pyridin-2-yl)-L-alanine;
N-[(2-ethyl-4-fluorophenyl)carbonyl]-3-(5-{3-[6-(methylamino)pyridin-2-yl]propyl}pyridin-2-yl)-L-alanine;
N-[(2,6-dichlorophenyl)carbonyl]-3-{6-[2-(5,6,7,8-tetrahydro-1,8-naphthyridin-2-yl)ethoxy]pyridin-3-yl}-L-alanine;
N-[(2-chlorophenyl)carbonyl]-3-{6-[2-(5,6,7,8-tetrahydro-1,8-naphthyridin-2-yl)ethoxy]pyridin-3-yl}-L-alanine;
N-[(2-chloro-4-fluorophenyl)carbonyl]-3-{6-[2-(5,6,7,8-tetrahydro-1,8-naphthyridin-2-yl)ethoxy]pyridin-3-yl}-L-alanine;
N-[(2-chloro-6-fluorophenyl)carbonyl]-3-{6-[2-(5,6,7,8-tetrahydro-1,8-naphthyridin-2-yl)ethoxy]pyridin-3-yl}-L-alanine;
N-[(2-chlorophenyl)carbonyl]-3-{5-[2-(5,6,7,8-tetrahydro-1,8-naphthyridin-2-yl)ethoxy]pyridin-2-yl}-L-alanine;
N-[(2-chloro-4-fluorophenyl)carbonyl]-3-{5-[2-(5,6,7,8-tetrahydro-1,8-naphthyridin-2-yl)ethoxy]pyridin-2-yl}-L-alanine;
N-[(2-chloro-6-fluorophenyl)carbonyl]-3-{5-[2-(5,6,7,8-tetrahydro-1,8-naphthyridin-2-yl)ethoxy]pyridin-2-yl}-L-alanine;
N-[(2-chloro-4-methoxyphenyl)carbonyl]-3-{5-[2-(5,6,7,8-tetrahydro-1,8-naphthyridin-2-yl)ethoxy]pyridin-2-yl}-L-alanine;
N-[(2-fluoro-4-methylphenyl)carbonyl]-3-{5-[2-(5,6,7,8-tetrahydro-1,8-naphthyridin-2-yl)ethoxy]pyridin-2-yl}-L-alanine;
N-[(2-ethyl-4-fluorophenyl)carbonyl]-3-{5-[2-(5,6,7,8-tetrahydro-1,8-naphthyridin-2-yl)ethoxy]pyridin-2-yl}-L-alanine;
N-[(2-chlorophenyl)carbonyl]-3-(5-{2-[6-(methylamino)pyridin-2-yl]ethoxy}pyridin-2-yl)-L-alanine;
N-[(2-chloro-4-fluorophenyl)carbonyl]-3-(5-{2-[6-(methylamino)pyridin-2-yl]ethoxy}pyridin-2-yl)-L-alanine;
N-[(2-chloro-6-fluorophenyl)carbonyl]-3-(5-{2-[6-(methylamino)pyridin-2-yl]ethoxy}pyridin-2-yl)-L-alanine;
N-[(2-chloro-4-methoxyphenyl)carbonyl]-3-(5-{2-[6-(methylamino)pyridin-2-yl]ethoxy}pyridin-2-yl)-L-alanine;
N-[(2-ethyl-4-fluorophenyl)carbonyl]-3-(5-{2-[6-(methylamino)pyridin-2-yl]ethoxy}pyridin-2-yl)-L-alanine;
N-[(2,6-dichlorophenyl)carbonyl]-3-(5-{2-[6-(methylamino)pyridin-2-yl]ethoxy}pyridin-2-yl)-L-alanine;
N-[(2,6-dichlorophenyl)carbonyl]-3-{5-[3-(5,6,7,8-tetrahydro-1,8-naphthyridin-2-yl)propyl]pyridin-2-yl}-L-alanine;
N-[(2-chlorophenyl)carbonyl]-3-{5-[3-(5,6,7,8-tetrahydro-1,8-naphthyridin-2-yl)propyl]thiophen-2-yl}-L-alanine;
N-[(2-chloro-4-fluorophenyl)carbonyl]-3-{5-[3-(5,6,7,8-tetrahydro-1,8-naphthyridin-2-yl)propyl]thiophen-2-yl}-L-alanine;
N-[(2-chloro-6-fluorophenyl)carbonyl]-3-{5-[3-(5,6,7,8-tetrahydro-1,8-naphthyridin-2-yl)propyl]thiophen-2-yl}-L-alanine;
N-[(2,6-dichlorophenyl)carbonyl]-3-{5-[3-(5,6,7,8-tetrahydro-1,8-naphthyridin-2-yl)propyl]thiophen-2-yl}-L-alanine;
N-[(2-chloro-6-fluorophenyl)carbonyl]-4-[3-(5,6,7,8-tetrahydro-1,8-naphthyridin-2-yl)propyl]-L-phenylalanine;
N-[2,6-dichlorophenyl)carbonyl]-4-[3-(5,6,7,8-tetrahydro-1,8-naphthyridin-2-yl)propyl]-L-phenylalanine;
N-[(2-chloro-4-fluorophenyl)carbonyl]-4-[3-(5,6,7,8-tetrahydro-1,8-naphthyridin-2-yl)propyl]-L-phenylalanine;
N-[(2-chlorophenyl)carbonyl]-3-{5-[3-(5,6,7,8-tetrahydro-1,8-naphthyridin-2-yl)propyl]pyridin-2-yl}-L-alanine;
N-[(2-fluoro-6-methylophenyl)carbonyl]-3-{5-[3-(5,6,7,8-tetrahydro-1,8-naphthyridin-2-yl)propyl]pyridin-2-yl}-L-alanine; and
N-[(2-chloro-4-fluorophenyl)carbonyl]-3-{5-[3-(5,6,7,8-tetrahydro-1,8-naphthyridin-2-yl)propyl]pyridin-2-yl}-L-alanine;
or a pharmaceutically acceptable salt or prodrug thereof.
In another embodiment of the invention there is provided a compound of the formula I selected from:
N-(2,6-dichlorobenzoyl)-4-[4-(pyridin-2-ylamino)piperidin-1-yl]-L-phenylalanine;
N-(2,6-dichlorobenzoyl)-4-(4-{[6-(methylamino)pyridin-2-yl]methyl}piperidin-1-yl)-L-phenylalanine;
N-(2,6-dichlorobenzoyl)-4-[(5,6,7,8-tetrahydro-1,8-naphthyridin-2-ylacetyl)amino]-L-phenylalanine;
N-(2,6-dichlorobenzoyl)-4-{[(5,6,7,8-tetrahydro-1,8-naphthyridin-3-ylmethyl)amino]carbonyl}-L-phenylalanine; and
N-(2,6-dichlorobenzoyl)-4-(3-{[6-(methylamino)pyridin-2-yl]methyl}azetidin-1-yl)-L-phenylalanine;

or a pharmaceutically acceptable salt or prodrug thereof.

In another embodiment of the invention there is provided a compound of the formula I selected from:
N-[(2-chloro-6-fluorophenyl)carbonyl]-3-{5-[3-(5,6,7,8-tetrahydro-1,8-naphthyridin-2-yl)propyl]pyridin-2-yl}-L-alanine;
N-[(2,6-dichlorophenyl)carbonyl]-3-{6-[3-(5,6,7,8-tetrahydro-1,8-naphthyridin-2-yl)propyl]pyridin-3-yl}-L-alanine;
N-[(2-chlorophenyl)carbonyl]-3-{6-[3-(5,6,7,8-tetrahydro-1,8-naphthyridin-2-yl)propyl]pyridin-3-yl}-L-alanine;
N-[(2-chloro-4-fluorophenyl)carbonyl]-3-{6-[3-(5,6,7,8-tetrahydro-1,8-naphthyridin-2-yl)propyl]pyridin-3-yl}-L-alanine; and
N-[(2-chloro-6-fluorophenyl)carbonyl]-3-{6-[3-(5,6,7,8-tetrahydro-1,8-naphthyridin-2-yl)propyl]pyridin-3-yl}-L-alanine;

or a pharmaceutically acceptable salt or prodrug thereof.

In another embodiment of the invention there is provided a compound of the formula I selected from:
N-[(2-chloro-6-fluorophenyl)carbonyl]-3-{5-[3-(5,6,7,8-tetrahydro-1,8-naphthyridin-2-yl)propyl]pyridin-2-yl}-L-alanine;
N-[(2,6-dichlorophenyl)carbonyl]-3-{6-[3-(5,6,7,8-tetrahydro-1,8-naphthyridin-2-yl)propyl]pyridin-3-yl}-L-alanine;
N-[(2-chlorophenyl)carbonyl]-3-{6-[3-(5,6,7,8-tetrahydro-1,8-naphthyridin-2-yl)propyl]pyridin-3-yl}-L-alanine;
N-[(2-chloro-4-fluorophenyl)carbonyl]-3-{6-[3-(5,6,7,8-tetrahydro-1,8-naphthyridin-2-yl)propyl]pyridin-3-yl}-L-alanine; and
N-[(2-chloro-6-fluorophenyl)carbonyl]-3-{6-[3-(5,6,7,8-tetrahydro-1,8-naphthyridin-2-yl)propyl]pyridin-3-yl}-L-alanine;

or a pharmaceutically acceptable salt or prodrug thereof.

In another embodiment of the invention there is provided a compound of the formula I selected from:
ethyl N-[(2,6-dichlorophenyl)carbonyl]-O-[3-(pyridin-2-ylamino)propyl]-L-tyrosinate;
1-methylethyl N-[(2,6-dichlorophenyl)carbonyl]-O-[3-(pyridin-2-ylamino)propyl]-L-tyrosinate;
2-morpholin-4-ylethyl N-[(2,6-dichlorophenyl)carbonyl]-O-[3-(pyridin-2-ylamino)propyl]-L-tyrosinate;
2-(2-oxopyrrolidin-1-yl)ethyl N-[(2,6-dichlorophenyl)carbonyl]-O-[3-(pyridin-2-ylamino)propyl]-L-tyrosinate;
2-(2-oxopiperidin-1-yl)ethyl N-[(2,6-dichlorophenyl)carbonyl]-O-[3-(pyridin-2-ylamino)propyl]-L-tyrosinate;
2-(2-oxoazepan-1-yl)ethyl N-[(2,6-dichlorophenyl)carbonyl]-O-[3-(pyridin-2-ylamino)propyl]-L-tyrosinate;
4-(glycylamino)phenyl N-[(2,6-dichlorophenyl)carbonyl]-O-[3-(pyridin-2-ylamino)propyl]-L-tyrosinate;
2-hydroxyethyl N-[(2,6-dichlorophenyl)carbonyl]-O-[3-(pyridin-2-ylamino)propyl]-L-tyrosinate;
2-(dimethylamino)-2-oxoethyl N-[(2,6-dichlorophenyl)carbonyl]-O-[3-(pyridin-2-ylamino)propyl]-L-tyrosinate; and
2-(diethylamino)ethyl N-[(2,6-dichlorophenyl)carbonyl]-O-[3-(pyridin-2-ylamino)propyl]-L-tyrosinate;

or a pharmaceutically acceptable salt or prodrug thereof.

Synthesis

The compounds of the present invention can be prepared in a number of ways using methods analogous to well known methods of organic synthesis. More specifically, the novel compounds of this invention may be prepared using the reactions and techniques described herein. In the description of the synthetic methods described below, it is to be understood that all proposed reaction conditions, including choice of solvent, reaction atmosphere, reaction temperature, duration of the experiment and workup procedures, are chosen to be the conditions standard for that reaction. It is understood by one skilled in the art of organic synthesis that the functionality present on various portions of the molecule must be compatible with the reagents and reactions proposed. Such restrictions to the substituents, which are not compatible with the reaction conditions, will be apparent to one skilled in the art and alternate methods must then be used.

It will be appreciated that during certain of the following processes certain substituents may require protection to prevent their undesired reaction. The skilled chemist will appreciate when such protection is required, and how such protecting groups may be put in place, and later removed.

For examples of protecting groups see one of the many general texts on the subject, for example, ‘Protective Groups in Organic Synthesis’ by Theodora Green (publisher: John Wiley & Sons). Protecting groups may be removed by any convenient method as described in the literature or known to the skilled chemist as appropriate for the removal of the protecting group in question, such methods being chosen so as to effect removal of the protecting group with minimum disturbance of groups elsewhere in the molecule.

Thus, if reactants include, for example, groups such as amino, carboxy or hydroxy it may be desirable to protect the group in some of the reactions mentioned herein.

A suitable protecting group for an amino or alkylamino group is, for example, an acyl group, for example an alkanoyl group such as acetyl, an alkoxycarbonyl group, for example a methoxycarbonyl, ethoxycarbonyl or t-butoxycarbonyl group, an arylmethoxycarbonyl group, for example benzyloxycarbonyl, or an aroyl group, for example benzoyl. The deprotection conditions for the above protecting groups necessarily vary with the choice of protecting group. Thus, for example, an acyl group such as an alkanoyl or alkoxycarbonyl group or an aroyl group may be removed for example, by hydrolysis with a suitable base such as an alkali metal hydroxide, for example lithium or sodium hydroxide. Alternatively an acyl group such as a t-butoxycarbonyl group may be removed, for example, by treatment with a suitable acid as hydrochloric, sulfuric or phosphoric acid or trifluoroacetic acid and an arylmethoxycarbonyl group such as a benzyloxycarbonyl group may be removed, for example, by hydrogenation over a catalyst such as palladium-on-carbon, or by treatment with a Lewis acid for example boron tris(trifluoroacetate). A suitable alternative protecting group for a primary amino group is, for example, a phthaloyl group which may be removed by treatment with an alkylamine, for example dimethylaminopropylamine, or with hydrazine.

A suitable protecting group for a hydroxy group is, for example, an acyl group, for example an alkanoyl group such as acetyl, an aroyl group, for example benzoyl, or an arylmethyl group, for example benzyl. The deprotection conditions for the above protecting groups will necessarily vary with the choice of protecting group. Thus, for example, an acyl group such as an alkanoyl or an aroyl group may be removed, for example, by hydrolysis with a suitable base such as an alkali metal hydroxide, for example lithium, sodium hydroxide or ammonia. Alternatively an arylmethyl group such as a benzyl group may be removed, for example, by hydrogenation over a catalyst such as palladium-on-carbon.

A suitable protecting group for a carboxy group is, for example, an esterifying group, for example a methyl or an ethyl group which may be removed, for example, by hydrolysis with a base such as sodium hydroxide, or for example a t-butyl group which may be removed, for example, by treatment with an acid, for example an organic acid such as trifluoroacetic acid, or for example a benzyl group which may be removed, for example, by hydrogenation over a catalyst such as palladium-on-carbon.

Resins may also be used as a protecting group.

The protecting groups may be removed at any convenient stage in the synthesis using conventional techniques well known in the chemical art.

Compounds of the formula I, or pharmaceutically-acceptable salts or prodrugs thereof, may be prepared by any process known to be applicable to the preparation of chemically-related compounds. Such processes, when used to prepare a compound of the formula I, or a pharmaceutically-acceptable salt or prodrug thereof, are provided as a further feature of the invention and are illustrated by the following representative examples. Necessary starting materials may be obtained by standard procedures of organic chemistry (see, for example, Advanced Organic Chemistry (Wiley-Interscience), Jerry March). The preparation of such starting materials is described within the accompanying non-limiting Examples. Alternatively, necessary starting materials are obtainable by analogous procedures to those illustrated which are within the ordinary skill of an organic chemist.

The present invention also provides that compounds of the formula I, or pharmaceutically acceptable salts or prodrugs thereof, can be prepared by a process (a) to (m) as follows (wherein the variables are as defined above unless otherwise stated):

Process (a) for the preparation of those compounds of formula I wherein Z is N(R²⁶), O or S, by reacting a compound of the formula II:

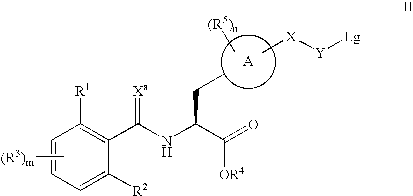

wherein R¹, R², R³, R⁴, R⁵, X^{a}, A, Z, Y, n and m are as hereinbefore defined, except any functional group is protected if necessary, and

Lg is a displaceable group,

with a compound of the formula III:
R⁶-ZH III

wherein R⁶ and Z are as hereinbefore defined, except any functional group is protected if necessary; or

Process (b) for the preparation of those compounds of the formula I wherein X is O, the coupling of a compound of the formula IV:

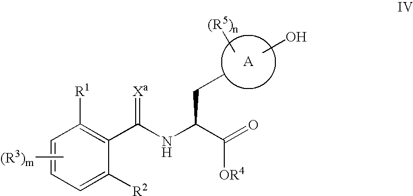

wherein R¹, R², R³, R⁴, R⁵, X^{a}, A, n and m are as hereinbefore defined, except any functional group is protected if necessary,

with a compound of the formula V:
R⁶-Z-Y—OH V

wherein R⁶, Y and Z are as hereinbefore defined, except any functional group is protected if necessary; or

Process (c) for the preparation of those compounds of formula I wherein X is O, N(R²⁶) or S by reacting a compound of the formula VI:

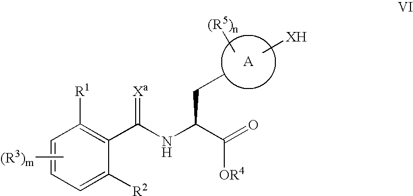

wherein R¹, R², R³, R⁴, R⁵, X^{a}, A, n and m are as hereinbefore defined, except any functional group is protected if necessary,

with a compound of the formula VII:
R⁶-Z-Y-Lg¹ VII

wherein R⁶, Y and Z are as hereinbefore defined, except any functional group is protected if necessary, and

Lg¹ is a displaceable group; or

Process (d) for the preparation of those compounds of the formula I wherein Z is —C═C—, —C≡C— or the group —Y-Z is alkylene the reaction of a coupling of a compound of the formula VIII:

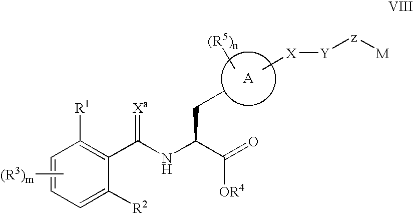

wherein R¹, R², R³, R⁴, R⁵, A, X^{a}, X, Y n and m are as hereinbefore defined, except any functional group is protected if necessary,

and M is a suitable displaceable group,

with a compound of the formula R⁶Lg²,

wherein R⁶ is as hereinbefore defined, except any functional group is protected if necessary, and

Lg² is a displaceable group; or

Process (e) for the preparation of those compounds of the formula I wherein X is N(R²⁶)C(O), the coupling of a compound of the formula IX:

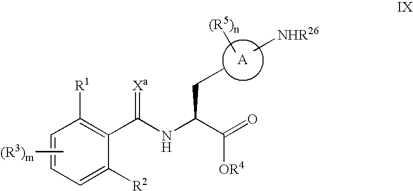

wherein R¹, R², R³, R⁴, R⁵, R²⁶, X^{a}, A, n and m are as hereinbefore defined, except any functional group is protected if necessary,

with a compound of the formula X, or a reactive derivative thereof:
R⁶-Z-Y—COOH X

wherein R⁶, Y and Z are as hereinbefore defined, except any functional group is protected if necessary; or

Process (f) for compounds of formula I where X^{a} is oxygen, the coupling of a compound of the formula XI:

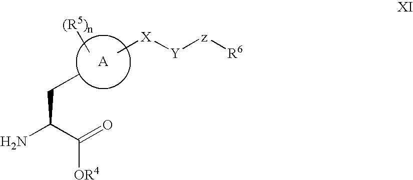

wherein R⁴, R⁵, R⁶, A, X, Y, Z and n are as hereinbefore defined, except any functional group is protected if necessary,

with a compound of the formula XII, or a reactive derivative thereof:

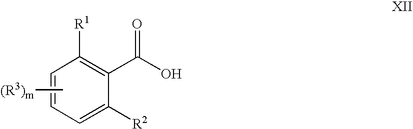

wherein R¹, R², R³ and m are as hereinbefore defined, except any functional group is protected if necessary; or

Process (g) for the preparation of those compounds of the formula I wherein X is C(O)N(R²⁶), the coupling of a compound of the formula XIII, or a reactive derivative thereof:

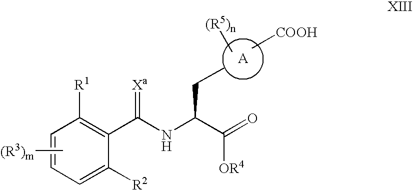

wherein R¹, R², R³, R⁴, R⁵, X^{a}, A, n and m are as hereinbefore defined, except any functional group is protected if necessary,

with a compound of the formula XIV:
R⁶-Z-Y—NH(R²⁶) XIV

wherein R⁶, Y, Z and R²⁶ are as hereinbefore defined, except any functional group is protected if necessary; or

Process (h) for the preparation of those compounds of the formula I wherein X is N(R²⁶), O or S, or where X is a direct bond and Y is a heterocyclic group which is linked to the group A in formula I via a ring nitrogen atom, the coupling of a compound of the formula XV:

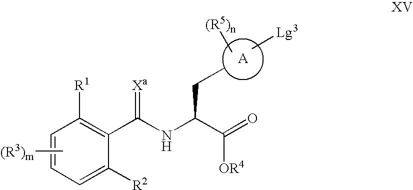

wherein R¹, R², R³, R⁴, R⁵, X^{a}, A, n and m are as hereinbefore defined, except any functional group is protected if necessary, and

Lg³ is a suitable displaceable group,

with a compound of the formula XVI:
R⁶-Z-Y—X—H XVI

wherein R⁶, X, Y and Z are as hereinbefore defined, except any functional group is protected if necessary; or

Process (i) for the preparation of those compounds of the formula I wherein X is —C═C—, —C≡C— or the group —X—Y is alkylene the coupling of a compound of the formula XV:

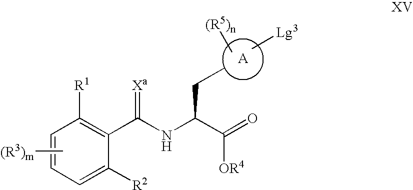

wherein R¹, R², R³, R⁴, R⁵, X^{a}, A, Lg³, n and m are as hereinbefore defined, except any functional group is protected if necessary,

with a compound of the formula XVII:
R⁶-Z-Y—X-M XVII

wherein R⁶, Y and Z are as hereinbefore defined, except any functional group is protected if necessary,

and M is a suitable displaceable group; or

Process (j) for the preparation of those compounds of the formula I wherein Z is N(R²⁶), the coupling of a compound of the formula XVIII:

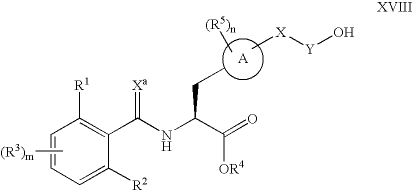

wherein R¹, R², R³, R⁴, R⁵, A, X^{a}, X, Y, n and m are as hereinbefore defined, except any functional group is protected if necessary,

with a compound of the formula XIX:
R⁶—N(R²⁶)H XIX

wherein R⁶ and R²⁶ are as hereinbefore defined, except any functional group is protected if necessary; or

Process (k) for the preparation of those compounds of formula I wherein Z is N(R²⁶), O or S, by reacting a compound of the formula XX:

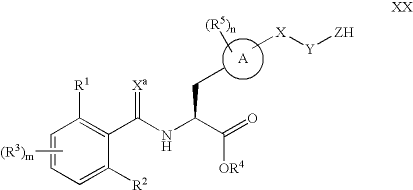

wherein R¹, R², R³, R⁴, R⁵, A, X^{a}, X, Y, Z, n and m are as hereinbefore defined, except any functional group is protected if necessary,

with a compound of the formula XXI:
R⁶-Lg XXI

wherein R⁶ is as hereinbefore defined, except any functional group is protected if necessary, and

Lg is a displaceable group; or

Process (l) for the preparation of those compounds of the formula I wherein the group —X—Y-Z- contains an alkylene chain of at least 3 carbon atoms in length, the hydrogenation of the product of Process (d) or (i) described herein; or

Process (m) for the preparation of those compounds of the formula I where X^{a} is a sulfur, by reacting a compound of formula XXII:

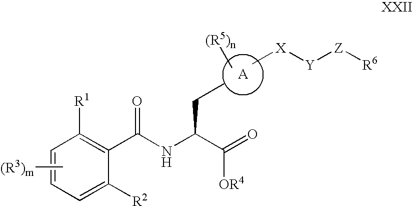

wherein R¹, R², R³, R⁴, R⁵, R⁶, A, X, Y, Z, n and m are as hereinbefore defined, except any functional group is protected if necessary,

with a thiation reagent such as S₈ or Lawesson reagent (2,4-bis-(4-methoxyphenyl)-1,3-dithia-2,4-diphosphetane 2,4-disulfide); or

Process (n) for the preparation of those compounds of the formula I wherein —X—Y-Z-contains a (1-6C)alkoxy or substituted (1-6C)alkoxy group or a (1-6C)alkylamino or substituted (1-6C)alkylamino group, the alkylation, conveniently in the presence of a suitable base as defined hereinbefore, of the corresponding alcohol or amine in which X, Y or Z contains a hydroxy group or a primary or secondary amino group as appropriate; or a reductive amination in which X, Y or Z contains a primary or secondary amino group as appropriate;

Process (o) R⁶ is 5,6,7,8-tetrahydro-1,8-naphthyridin-2-yl; the reduction of a compound of the formula XXIV: 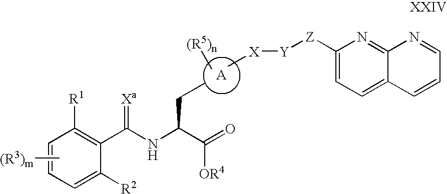
wherein A, R¹, R², R³, R⁴, R⁵, X, Y, Z, n and m are as hereinbefore defined, except any functional group is protected if necessary;

and thereafter, if necessary (in any order):

(i) converting a compound of the formula I into another compound of the formula I;

(ii) removing any protecting groups; and

(iii) forming a pharmaceutically acceptable salt of the compound of formula I.

Suitably in any one of processes (a) to (1), X^{a} is oxygen and once complete process (m) is conducted. However, if required thiation can be carried out on any of the intermediates used in the process to ensure that XV is sulphur in the final product.

Specific conditions for the above reactions are as follows.

Reaction Conditions for Process (a)

A convenient displaceable group Lg is, for example, a halo, alkanesulfonyloxy or arylsulfonyloxy group, for example a chloro, bromo, methanesulfonyloxy, trifluoromethanesulfonyloxy, 4-nitrobenzenesulfonyloxy or toluene-4-sulfonyloxy group.

The reaction is advantageously carried out in the presence of base. A suitable base is, for example, an organic amine base such as, for example, pyridine, 2,6-lutidine, collidine, 4-dimethylaminopyridine, triethylamine, N-methylmorpholine or diazabicyclo[5.4.0]undec-7-ene, or for example, an alkali metal or alkaline earth metal carbonate or hydroxide, for example sodium carbonate, potassium carbonate, cesium carbonate, calcium carbonate, sodium hydroxide or potassium hydroxide. Alternatively such a base is, for example, an alkali metal hydride, for example sodium hydride, an alkali metal or alkaline earth metal amide, for example sodium amide or sodium bis(trimethylsilyl)amide, or a sufficiently basic alkali metal halide, for example cesium fluoride or sodium iodide. The reaction is suitably effected in the presence of an inert solvent or diluent, for example a dipolar aprotic solvent such as N N-dimethylformamide, N,N-dimethylacetamide, N-methylpyrrolidin-2-one or dimethylsulfoxide. The reaction is conveniently effected at a temperature in the range, for example, 10 to 150° C. (or the boiling point of the solvent), suitably in the range 20 to 90° C.

Compounds of the formula II may be prepared using methods well known to those skilled in organic chemistry. Representative methods are illustrated in the Examples described herein.

Compounds of the formula III are commercially available, or they are known in the literature, or they can be prepared by standard processes known in the art.

Reaction Conditions for Process (b)

The coupling reaction is suitably carried out using the Mitsunobu reaction. Suitable Mitsunobu conditions include, for example, reaction in the presence of a suitable tertiary phosphine and a di-alkylazodicarboxylate in an organic solvent such as an ether, for example THF or a halogenated solvent such as methylene chloride. The reaction is suitably carried out in the temperature range −15° C. to 60° C., for example at or near ambient temperature. A suitable tertiary phosphine includes for example tri-n-butylphosphine or particularly tri-phenylphosphine. A suitable di-alkylazodicarboxylate includes for example diethyl azodicarboxylate (DEAD) or di-tert-butyl azodicarboxylate (DTAD) or azodicarbonyldipiperidine (DPAD or ADDP). Details of Mitsunobu reactions are contained in Tet. Letts., 31, 699, (1990); The Mitsunobu Reaction, D. L. Hughes, Organic Reactions, 1992, Vol. 42, 335-656 and Progress in the Mitsunobu Reaction, D. L. Hughes, Organic Preparations and Procedures International, 1996, Vol. 28, 127-164.

Compounds of the formula IV may be prepared using methods well known to those skilled in organic chemistry. Representative methods are illustrated in the Examples described herein.

Compounds of the formula V are commercially available, or they are known in the literature, or they can be prepared by standard processes known in the art.

Reaction Conditions for Process (c)

Suitable leaving groups represented by Lg¹ include those described above for Lg in Process (a), for example halo, such as bromo. The reaction is suitably carried out in the presence of a base, for example a base as hereinbefore described in relation to Process (a) such as an alkali metal or alkaline earth metal carbonate for example potassium carbonate.

The reaction is suitably effected in the presence of an inert solvent or diluent, for example a dipolar aprotic solvent such as N N-dimethylformamide, N,N-dimethylacetamide, N-methylpyrrolidin-2-one or dimethylsulfoxide. The reaction is conveniently effected at a temperature in the range, for example, 10 to 150° C. (or the boiling point of the solvent), suitably in the range 20 to 90° C.

Compounds of the formula VI may be prepared using methods well known to those skilled in organic chemistry. Representative methods are illustrated in the Examples described herein.

Compounds of the formula VII are commercially available, or they are known in the literature, or they can be prepared by standard processes known in the art.

Reaction Conditions for Process (d)

Suitable coupling reactions are well known to those of ordinary skill in the art of organic chemistry. For example coupling under Heck, Suzuki, Stille, Negishi or when Z is —C≡C—, Sonogashira coupling conditions.

A Heck reaction is suitable for those compounds where Z is —C═C═ and M in formula VIII is M. For a Heck reaction, a suitable displaceable group Lg² is, for example, as hereinbefore defined for Lg, particularly a halo such as, for example, bromo or iodo; and M is H.

Suitable conditions for the Heck reaction are well known such as those described in Syn Lett, 12, 1877 (2005). For example, reaction in the presence of a tertiary base, and a palladium-based catalyst in an inert solvent. The reaction is suitably carried out in the temperature range of 25° C. to 150° C. under thermal or microwave conditions. A suitable tertiary base includes for example triethylamine, N,N-diisopropylethylamine. A suitable palladium catalyst includes palladium(II) acetate in the presence of a phosphine ligand such as tri-phenylphosphine, tri-o-tolylphosphine (Hermman's catalyst), tri-n-butylphosphine. Suitable solvents include N,N-dimethylformamide, tetrahydrofuran, 1,4-dioxane, N,N-dimethylacetamide and 1,2-dimethyoxyethane.

When a Stille coupling is used, Lg² is suitably a halo such as chloro, bromo or iodo, or pseudo halide such as a triflate; and M is a suitable stannane, for example a trialkylstannane such as tributylstannyl, (Bu)₃Sn—. The Stille coupling is carried out in the presence of a suitable palladium catalyst. Suitably the reaction is carried out in a polar solvent such as DMF.

When a Suzuki reaction is used Lg² is suitably a halo such as chloro, bromo or iodo, or pseudo halide such as a triflate; and M is boronic acid or a suitable derivative thereof. For example, M may be a boronic acid ester, potassium trifluoroborate or an organoborane. The coupling reaction is performed in the presence of a palladium catalyst and a suitable base. Suitable bases are as hereinbefore defined.

When a Negishi coupling is used Lg² is suitably a halo such as chloro, bromo or iodo, triflate or acetoxy; and M is an organo zinc group such as a zinc halide, for example ZnI. The reaction is performed in the presence of a suitable palladium or nickel catalyst. The reaction is conveniently performed in the presence of an inert organic solvent such as NMP, THF or DMA.

When Z is —C≡C—, Sonogashira coupling conditions may also be used, wherein Lg is suitably a halo such as chloro, bromo or iodo or triflate; and M is hydrogen. The reaction is performed in the presence of a suitable palladium catalyst, such as a Pd(0) catalyst or bis triphenylphosphine palladium(II)chloride, and a suitable copper (I) catalyst, such as a copper(I)halide, for example copper iodide. The reaction is suitably performed in the presence of a suitable base, for example a tertiary base such as triethylamine. The reaction is suitably carried out in the temperature range of 25° C. to 150° C. under thermal or microwave conditions. Suitable solvents include N,N-dimethylformamide, N,N-dimethylacetamide and toluene.

Suitable conditions for the Suzuki, Stille, Negishi and Sonogashira are well known and are described in, for example, “Metal-catalysed Cross-Coupling reactions Edited by Armin de Meijere and François Diederich; Wiley-VCH Verlag 2^{nd} Edition 2004.

As will be realised, generally the alternative coupling reactions are also expected to be suitable wherein M is on R⁶ and Lg² is attached to Z.

Compounds of the formula VIII, or corresponding compounds wherein Lg² is attached to Z may be prepared using methods well known to those skilled in organic chemistry. Representative methods are illustrated in the Examples described herein.

Compounds of the formula R⁶Lg² and R⁶-M are commercially available, or they are known in the literature, or they can be prepared by standard processes known in the art.

Reaction Conditions for Process (e)

The coupling reaction may be carried out using standard methods for the coupling of acids and amines. The coupling reaction is conveniently carried out in the presence of a suitable coupling reagent. Standard peptide coupling reagents known in the art can be employed as suitable coupling reagents for example O-(Benzotriazol-1-yl)-N,N,N′,N′-tetramethyluronium tetrafluoroborate (TBTU) or O-(7-Azabenzotriazol-1-yl)-N,N,N′,N′-tetramethyluronium hexafluoro-phosphate (HATU), or for example carbonyldiimidazole, dicyclohexylcarbodiimide and N-ethyl-N′-(3-dimethylaminopropyl)carbodiimide, optionally in the presence of a catalyst such as dimethylaminopyridine, 4-pyrrolidinopyridine or 2-hydroxy-pyridine-N-oxide, optionally in the presence of a base for example triethylamine, N-methylmorpholine, pyridine, or 2,6-di-alkyl-pyridines such as 2,6-lutidine or 2,6-di-tert-butylpyridine. The reaction is conveniently performed in the present of a suitable inert solvent. Suitable solvents include N,N-dimethylacetamide, dichloromethane, benzene, tetrahydrofuran and N,N-dimethylformamide. The coupling reaction is conveniently performed at a temperature in the range of −40 to 40° C.

A “reactive derivative” of the acid of the formula X is a carboxylic acid derivative that will react with the amine of the formula IX to give the corresponding amide. A suitable reactive derivative of a carboxylic acid of the formula X is, for example, an acyl halide, for example an acyl chloride formed by the reaction of the acid and an inorganic acid chloride, for example thionyl chloride; a mixed anhydride, for example an anhydride formed by the reaction of the acid and a chloroformate such as isobutyl chloroformate; an active ester, for example an ester formed by the reaction of the acid and a phenol such as pentafluorophenol in the presence of a suitable coupling agent (such as an carbodiimide), an ester such as pentafluorophenyl trifluoroacetate or an alcohol such as methanol, ethanol, isopropanol, butanol or N-hydroxybenzotriazole; or an acyl azide, for example an azide formed by the reaction of the acid and azide such as diphenylphosphoryl azide; an acyl cyanide, for example a cyanide formed by the reaction of an acid and a cyanide such as diethylphosphoryl cyanide. The reaction of such reactive derivatives of carboxylic acid with amines is well known in the art, for example they may be reacted in the presence of a base, such as those described above, and in a suitable solvent, such as those described above. The reaction may conveniently be performed at a temperature as described above.

Compounds of the formula IX may be prepared using methods well known to those skilled in organic chemistry. Representative methods are illustrated in the Examples described herein.

Compounds of the formula X are commercially available, or they are known in the literature, or they can be prepared by standard processes known in the art.

Reaction Conditions for Process (f)

The coupling is suitably carried out under analogous conditions to those described above in relation to Process (e) for the coupling of acids and amines. Examples of reactive derivatives of the acid of formula XII are as described in relation to Process (e).

Compounds of the formula XI may be prepared using methods well known to those skilled in organic chemistry. Representative methods are illustrated in the Examples described herein.

Compounds of the formula XII are commercially available, or they are known in the literature, or they can be prepared by standard processes known in the art.

Reaction Conditions for Process (g)

The coupling may be carried out under analogous conditions to those described above in relation to Process (e) for the coupling of acids and amines. Suitable reactive derivatives of the compound of the formula XIII are carboxylic acid derivatives such as those described in relation to reactive derivatives of the compound of formula XII described hereinbefore.

Compounds of the formula XIII may be prepared using methods well known to those skilled in organic chemistry.

Compounds of the formula XIV are commercially available, or they are known in the literature, or they can be prepared by standard processes known in the art.

Reaction Conditions for Process (h)

Lg³ is a suitable displaceable group as hereinbefore defined in relation to Lg such as trifluoromethanesulfonyloxy or toluene-4-sulfonyloxy group or particularly halo such as bromo or iodo.

The coupling reaction may be carried out under known conditions for the coupling of aromatic groups, for example using an Ullmann type reaction. Suitable conditions for the Ullmann type reaction include, for example, reaction in the presence of a base, a copper (I)-based catalyst in an inert solvent. The reaction is suitably carried out in the temperature range of 25° C. to 150° C. under thermal or microwave conditions. A suitable base includes for example cesium carbonate. A suitable catalyst includes copper iodide in the presence of a ligand such as L-proline or 1,10-phenanthroline. Suitable solvents include NAN-dimethylformamide, N,N-dimethylacetamide and dimethylsulfoxide.

When X is N(R²⁶), the coupling may also be performed using the Buchwald reaction. Suitable conditions for the Buchwald reaction include, for example, reaction in the presence of a suitable base, a palladium-based catalyst in an inert solvent. The reaction is suitably carried out in the temperature range of 25° C. to 150° C. under thermal or microwave conditions. A suitable base includes for example an alkoxide base such as NaOt-Bu or a carbonate such as cesium carbonate. A suitable palladium catalyst includes bis(dibenzylideneacetone)palladium(0) in the presence of a phosphine ligand such as xantphos. Suitable solvents include N,N-dimethylformamide, N,N-dimethylacetamide, dimethylsulfoxide and toluene.

Alternatively, when X is O Lg³ may be boronic acid or a suitable derivative thereof. For example, Lg³ may is boronic acid. Suitably the coupling is performed in the presence of a copper(II)-based catalyst, optionally in the presence of oxygen

Compounds of the formula XV may be prepared using methods well known to those skilled in organic chemistry.

Compounds of the formula XVI are commercially available, or they are known in the literature, or they can be prepared by standard processes known in the art.

Reaction Conditions for Process (i)

The coupling reaction may be carried out under Heck, Suzuki, Stille, Negishi or when Z is —C≡C—, Sonogashira coupling conditions as described in relation to Process (d) above. In an alternative reaction the coupling are expected to be suitable for coupling where M is on ring A in formula XV and Lg³ is on X in the compound of formula XVII.

Compounds of the formula XVII are commercially available, or they are known in the literature, or they can be prepared by standard processes known in the art.

Reaction Conditions for Process (j)

The coupling reaction may be carried out using the Mitsunobu reaction as described in relation to Process (b).

Compounds of the formula XVIII may be prepared using methods well known to those skilled in organic chemistry. Representative methods are illustrated in the Examples described herein.

Compounds of the formula XIX are commercially available, or they are known in the literature, or they can be prepared by standard processes known in the art.

Reaction Conditions for Process (k)

Suitable displaceable groups represented by Lg include those described in relation to Process (a), such as halo, for example chloro. The reaction may be performed under analogous conditions to those described for Process (a), conveniently in the presence of a suitable base such as a carbonate, for example sodium carbonate.

Compounds of the formula XX may be prepared using methods well known to those skilled in organic chemistry. Representative methods are illustrated in the Examples described herein.

Compounds of the formula XXI are commercially available, or they are known in the literature, or they can be prepared by standard processes known in the art.

Reaction Conditions for Process (l)

Hydrogenation conditions are well known in the art, and may include hydrogenation in the presence of a suitable catalyst such as a platinum on carbon or palladium on charcoal.

Reaction Conditions for Process (m)

Suitable conditions for the transformation of an amide into a thioamide include, for example, reaction in the presence of Lawesson reagent or S₈ in an inert solvent. The reaction is suitably carried out in the temperature range of 25° C. to 150° C. under thermal or microwave conditions. Suitable solvents include for instance toluene. Compounds of the formula XXII may be prepared using any of the processes (a) to (1) described above.

Reaction conditions for Process (n)

A suitable alkylating agent is, for example, any agent known in the art for the alkylation of hydroxy to alkoxy or substituted alkoxy, or for the alkylation of amino to alkylamino or substituted alkylamino, for example an alkyl or substituted alkyl halide, for example a (1-6C)alkyl chloride, bromide or iodide or a substituted (1-6C)alkyl chloride, bromide or iodide, conveniently in the presence of a suitable base as defined hereinbefore, in a suitable inert solvent or diluent as defined hereinbefore and at a temperature in the range, for example, 10 to 140° C., conveniently at or near ambient temperature. Conveniently for the production of those compounds of the formula I wherein —X—Y-Z-contains a (1-6C)alkylamino or substituted (1-6C)alkylamino group, a reductive amination reaction may be employed. For example, for the production of those compounds of the Formula I wherein X, Y or Z contains a N-alkyl group, the corresponding compound containing a N—H group may be reacted with formaldehyde (to give an N-methyl group), an appropriate aldehyde (to give an N-alkyl group) or an appropriate ketone (to give a N-substituted alkyl group) in the presence of a suitable reducing agent. A suitable reducing agent is, for example, a hydride reducing agent, for example an alkali metal aluminium hydride such as lithium aluminium hydride or, preferably, an alkali metal borohydride such as sodium borohydride, sodium cyanoborohydride, sodium triethylborohydride, sodium trimethoxyborohydride and sodium triacetoxyborohydride. The reaction is conveniently performed in a suitable inert solvent or diluent, for example tetrahydrofuran and diethyl ether for the more powerful reducing agents such as lithium aluminium hydride, and, for example, methylene chloride or a protic solvent such as methanol and ethanol for the less powerful reducing agents such as sodium triacetoxyborohydride and sodium cyanoborohydride. The reaction is performed at a temperature in the range, for example, 10 to 80° C., conveniently at or near ambient temperature. Suitable reductive amination conditions are well known, for example as described in Abdel-Magid, Ahmed F.; Mehrman, Steven J. A Review on the Use of Sodium Triacetoxyborohydride in the Reductive Amination of Ketones and Aldehydes. Organic Process Research & Development (2006), 10(5), 971-1031; or Baxter, Ellen W.; Reitz, Allen B. Reductive aminations of carbonyl compounds with borohydride and borane reducing agents. Organic Reactions (New York) (2002), 59 1-714. CODEN: ORREAW ISSN:0078-6179. CAN 138:169565 AN 2002:450507 CAPLUS.

Reaction Conditions for Process (o)

The reduction may be performed using a suitable reducing agent, for example by hydrogenation in the presence of a suitable catalyst such as a platinum on carbon or palladium on charcoal catalyst as illustrated in the examples herein.

The compound of formula XXIV may conveniently be prepared by reacting the methyl ketone of formula XXIVa with the compound of formula XXIVb 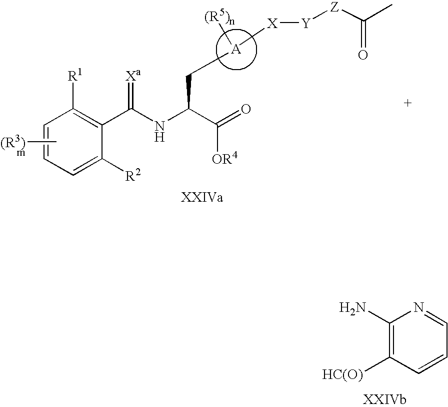
wherein A, R¹, R², R³, R⁴, R⁵, X, Y, Z, n and m are as hereinbefore defined, except any functional group is protected if necessary. The reaction is performed in the presence of a suitable base such as L-proline, or other an alkali metal hydroxide. Conveniently the reaction is performed in the presence of a suitable organic solvent such as an alcohol for example methanol, ethanol or isopropyl alcohol or an ether such as THF. Suitable reaction conditions and the preparation of the compound of formulae XXIVa are illustrated in the examples.

Compounds of the formula I may also be obtained by modifying a substituent in or introducing a substituent into another compound of formula I or a pharmaceutically acceptable salt or prodrug thereof. Suitable chemical transformations are well known to those in the art of organic chemistry. For example, when R⁴ is (1-6C)alkyl in a compound of formula I, the alkyl group may be replaced by hydrogen by hydrolysis of the compound of formula I to give another compound of formula I in which R⁴ is hydrogen. Suitably the hydrolysis is carried out in the presence of a suitable base such as lithium hydroxide. Further representative transformations include the reduction of a —C≡C— or —C═C— group in X, Y or Z to a —CH₂CH₂—. Suitable reducing conditions include for example hydrogenation in the presence of a suitable catalyst such as a platinum on carbon or palladium on charcoal. During these transformations, functional groups may be protected as required, and the protecting groups removed subsequently.

It will be appreciated that certain of the various ring substituents in the compounds of the present invention may be introduced by standard aromatic substitution reactions or generated by conventional functional group modifications either prior to or immediately following the processes mentioned above, and as such are included in the process aspect of the invention. Such reactions and modifications include, for example, introduction of a substituent by means of an aromatic substitution reaction, reduction of substituents, alkylation of substituents and oxidation of substituents. The reagents and reaction conditions for such procedures are well known in the chemical art. Particular examples of aromatic substitution reactions include the introduction of a nitro group using concentrated nitric acid, the introduction of an acyl group using, for example, an acyl halide and Lewis acid (such as aluminium trichloride) under Friedel Crafts conditions; the introduction of an alkyl group using an alkyl halide and Lewis acid (such as aluminium trichloride) under Friedel Crafts conditions; and the introduction of a halogeno group. Particular examples of modifications include the reduction of a nitro group to an amino group by for example, catalytic hydrogenation with a nickel catalyst or treatment with iron in the presence of hydrochloric acid with heating; oxidation of alkylthio to alkylsulfinyl or alkylsulfonyl; and the alkylation of an amino group by reductive amination. For example when R⁴ is other than hydrogen, esters of the compound of formula I may be prepared by reacting the compound of formula I wherein R⁴ is H with an alcohol R⁴OH using standard methods such as activation of the carboxylic acid with a carbodiimide followed by reaction with the alcohol or coupling under Mitsunobu conditions.

When a pharmaceutically acceptable salt of a compound of the formula I is required, for example an acid or base addition salt, it may be obtained by, for example, reaction of the compound of formula I with a suitable acid or base using a conventional procedure. Methods for the preparation of pharmaceutically acceptable salts are well known in the art. For example, following reaction of a compound of the formula I with an acid or base the required salt may be precipitated from solution by supersaturating the solution containing the compound of the formula I. Super saturation may be achieved using well-known techniques, for example by cooling the solution, by removing solvent by evaporation or by the addition of a suitable anti-solvent to precipitate the salt.

To facilitate isolation of a compound of the formula I during its preparation, the compound may be prepared in the form of a salt that is not a pharmaceutically acceptable salt. The resulting salt can then be modified by conventional techniques to give a pharmaceutically acceptable salt of the compound. Such salt modification techniques are well known and include, for example ion exchange techniques or re-precipitation of the compound from solution in the presence of a pharmaceutically acceptable counter ion as described above, for example by re-precipitation in the presence of a suitable pharmaceutically acceptable acid to give the required pharmaceutically acceptable acid addition salt of a compound of the formula I.

Stereoisomers of compounds of formula I may be separated using conventional techniques, e.g. chromatography or fractional crystallisation. The enantiomers may be isolated by separation of a racemate for example by fractional crystallisation, resolution or HPLC. The diastereoisomers may be isolated by separation by virtue of the different physical properties of the diastereoisomers, for example, by fractional crystallisation, HPLC or flash chromatography. Alternatively particular stereoisomers may be made by chiral synthesis from chiral starting materials under conditions which will not cause racemisation or epimerisation, or by derivatisation, with a chiral reagent. When a specific stereoisomer is isolated it is suitably isolated substantially free from other stereoisomers, for example containing less than 20%, particularly less than 10% and more particularly less than 5% by weight of other stereoisomers.

In the synthesis section above and hereafter, the expression “inert solvent” refers to a solvent which does not react with the starting materials, reagents, intermediates or products in a manner which adversely affects the yield of the desired product.

Persons skilled in the art will appreciate that, in order to obtain compounds of the invention in an alternative and in some occasions, more convenient manner, the individual process steps mentioned hereinbefore may be performed in different order, and/or the individual reactions may be performed at different stage in the overall route (i.e. chemical transformations may be performed upon different intermediates to those associated hereinbefore with a particular reaction).

Certain of the intermediates used in the above described processes for the preparation of compounds of the formula I form a further aspect of the invention, for example a compound selected from a compound of the formula II, VIII, X¹, XVIII, XX, XXII, XXIV and XXIVa defined hereinbefore, or a salt thereof, particularly a pharmaceutically acceptable salt thereof.

Biological Activity

The following assays can be used to measure the effects of the compounds of the present invention as a5b1 integrin inhibitors.

(a) In Vitro Binding Assay

The assay determined the ability of compounds to inhibit binding of α5β1 integrin to a cognate ligand, a fragment of human fibronectin. The assay used Origen technology (IGEN International) to measure the compound activity. Briefly, α5β1 integrin was coated onto epoxy-paramagnetic beads (Dynal Biotech UK, Bromborough, Wirral, CH62 3QL, UK, Catalogue No 140.11) and biotinylated-fibronectin ligand was coupled to strepatividin labeled Tag-NHS-Ester (BioVeris Corporation, Witney, Oxfordshire, OX28 4GE, UK, Catalogue No 110034). The ruthenium-labeled tag emits an electrochemiluminescence signal upon stimulation which is detected by the Origen reader. Thus, interaction of integrin and ligand causes association of bead and tag, and the resulting electrochemiluminescence signal reflects the level of integrin interaction with fibronectin.

20 μg of recombinant human α5β1 was coated onto 4×10(7) epoxy-paramagnetic beads according to manufacturers instructions at 4° C. for 24 hours. Integrin coating and corresponding activity was subsequently measured for each batch but typically, 25 ng of coated protein was used per assay well.

A DNA fragment encoding the domains 9-10 (amino-acids 1325-1509) of human fibronectin (Swiss-Prot Accession No. P02751) was isolated from cDNA libraries using standard molecular biology and PCR cloning techniques. The cDNA fragment was sub-cloned into a pT73.3 expression vector containing a GST-epitope tag (developed at AstraZeneca; Bagnall et al., Protein Expression and Purification, 2003, 27: 1-11). Following expression in E. coli, the expressed protein, termed Fn9-10, was purified using the GST-tag using standard purification techniques. The recombinant Fn9-10 was subsequently biotinylated using a EZ-link Sulfo-NHS-LC-Biotinylation kit (Perbio Science UK Ltd., Cramlington, Northumberland, NE23 1WA, UK, Catalogue No. 21335) and made to give a final concentration of approximately 1 mg/ml. Tag-NHS-Ester was labeled with streptavidin by incubation at room temperature following manufacturers instructions and buffer-exchanged into PBS to give a concentration of 0.5 mg/ml. Immediately prior to the assay, biotinylated-Fn9-10 and Streptavidin-labeled Tag were diluted in Assay Buffer to give a final concentrations of 0.6 ug/ml and 1.5 ug/ml respectively. The Fn9-10 and Tag solutions were then mixed together in equal volumes and incubated on ice for at least 30 minutes prior to the assay.

Test compounds were prepared as 10 mM stock solutions in DMSO (Sigma-Aldrich Company Ltd, Gillingham, Dorset SP8 4XT Catalogue No. 154938) and serially diluted with 4% DMSO to give a range of test concentrations at X⁴ required final concentration. Aliquots (20 μl) of each compound dilution were placed into each well of a 384-well round bottomed polypropylene plate (Matrix Technologies, Wilmslow, Cheshire, SK9 3LP, Catalogue No. 4340 384). Each plate also contained control wells: maximum signal was created using wells containing 20 μl of 4% DMSO, and minimum signal corresponding to no binding was created using wells containing 20 μl of 80 mM EDTA (Sigma Catalogue No. E7889).

For the assay, 25 ng (20 ul) of a5b 1-bead suspension and 40 μl of the Fn9-10/Tag pre-incubated solution were added to each well containing 20 μl of compound or control solution. Assay plates were then incubated at room temperature for a minimum of 6 hours before being analysed on an Origen plate reader. The minimum value was subtracted from all values, and the signal was plotted against compound concentration to generate IC₅₀ data.

(b) In Vitro Cell Adhesion Assay

The assay determined the ability of compounds to inhibit the α5β1 integrin mediated adhesion of K562 cells to the ligand, a fragment of human fibronectin. The human K562 erythroleukaemia cell line (LGC Promochem, Teddington, Middlesex, UK, Catalogue No. CCL-243) was routinely maintained in RPMI 1640 medium (Sigma-Aldrich Company Ltd, Gillingham, Dorset SP8 4XT, Catalogue No. R0883) containing 10% heat-inactivated foetal calf serum (PAA lab GmbH, Pasching, Austria Catalogue No. PAA-A15-043) and 1% glutamax-1 (Invitrogen Ltd. Paisley, UK Catalogue No. 35050-038) at 37° C. with 5% CO₂ at densities between 1×10⁵ and 1×10⁶ cells/ml.

A DNA fragment encoding the domains 9-10 (amino-acids 1325-1509) of human fibronectin (Swiss-Prot Accession No. P02751) was isolated from cDNA libraries using standard molecular biology and PCR cloning techniques. The cDNA fragment was sub-cloned into a pT7#3.3 expression vector containing a GST-epitope tag (developed at AstraZeneca; Bagnall et al., Protein Expression and Purification, 2003, 27: 1-11), and the fragment termed Fn9-10. Following expression in E. coli, the expressed protein was purified using the GST-tag using standard purification techniques.

For adhesion assay, a 96-well flat bottomed plate (Greiner Bio one ltd., Gloucester GL10 3 SX Catalogue No. 655101) was coated overnight at 4° C. with 100 μl of 20 μg/ml Fn9-10 ligand in Dulbecco's PBS (Gibco#14190-94). The plate was then washed twice with 200 μl of PBS and blocked with 100 μl of 3% BSA (SigmaA7888) in PBS for 1 hour at 37° C. The plates were then washed again 3 times with 200 μl of PBS and left empty.

Test compounds were prepared as 10 mM stock solutions in DMSO (Sigma-Aldrich Company Ltd, Gillingham, Dorset SP8 4XT Catalogue No. 154938) and serially diluted with HBSS (Hanks Buffered Salt solution (Gibco Catalogue No. 14170-088)/2% DMSO to give a range of test concentrations at twice required final concentration. Aliquots (50 μl) of each compound dilution were placed into each well of the Fn9-10 coated plates. Each plate also contained control wells: maximum adhesion signal was created using wells containing 50 μl HBSS/2% DMSO, and minimum signal corresponding to no adhesion was created using wells containing 50 μl HBSS/2% DMSO /20 mM EDTA (Sigma Catalogue No. E7889).

The K562 cells were cultured to 1×10⁶ cells/ml, and each culture suspension pooled. Cells were centrifuged at 1200 rpm for 2 mins, and the pellets washed with HBSS followed by HBSS/50 mM BEPES (Sigma Catalogue No. H0887). Cell pellets were pooled and re-suspended in HBSS/0.4 mM manganese chloride/50 mM BEPES (MnCl; Sigma Catalogue No. M1787) to give a final concentration of 4×10⁶ cells/ml.

The assay was initiated by the addition of 50 μl of cell suspension into each coated well (200,000 cells/well), thus resulting in final desired compound concentration and a final MnCl concentration of 0.2 mM. The plates were incubated for 45 minutes at 37° C. 5% CO₂. After this time, the solution was flicked off as waste, and the remaining cell layer carefully washed twice with 200 μl of PBS, and then fixed with 200 μl of 100% ethanol for 30 minutes.

After fixation, the ethanol was flicked off to waste and 100 μl of 0.1% Crystal violet stain was added to each well, and incubated at ambient temperature for 15 minutes. Excess stain was removed by rinsing 3 times under cold slow running water. The plates were blotted over tissue then solubilised by adding 50 μl of 1% Triton X100 (Sigma Catalogue No. T9284) and shaking at 500 rpm for 30 mins on plate shaker. Finally, 100 μl of deionised water was added to each well and the absorbance was determined at 590 nM on a spectrophotometer. The minimum value was subtracted from all values, and the absorbance signal was plotted against compound concentration to generate IC₅₀ data.

Although the pharmacological properties of the compounds of the formula I vary with structural change as expected, in general activity possessed by compounds of the formula I, may be demonstrated at the following concentrations or doses in one or more of the above tests (a) and (b):
Test (a):—IC₅₀ in the range, for example, 0.1 nM to 10 μM (preferred compounds have an IC₅₀ of less than 1 μM);
Test (b):—IC₅₀ in the range, for example, 1 nM to 20 μM (preferred compounds have an IC₅₀ of less than 5 μM);

By way of example, activity for the following compounds was observed:

| | | Binding | Adhesion |
|---|---|---|---|
| | | Assay (a) | Assay (b) |
| Example | Structure | (μM) | (μM) |
| 3 | 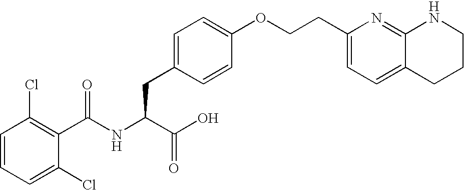 | 0.0004 (n = 9) | 0.002 (n = 8) |
| 29 | 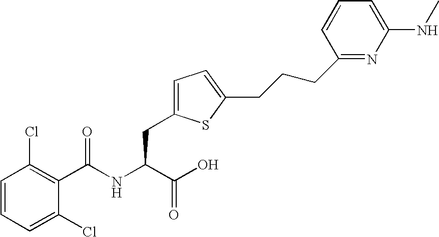 | 0.002 (n = 3) | 0.031 (n = 3) |

In the table n indicates the number of tests carried out on each compound and the IC₅₀ values shown represent the geometric mean of the measured IC₅₀ values for each compound.

We have found that the compounds N-(2,6-dichlorobenzoyl)-3-[2-(pyridin-2-ylamino)ethoxy]-L-phenylalanine and N-(2,6-dichlorobenzoyl)-3-[4-(pyridin-2-ylamino)butoxy]-L-phenylalanine had low activity in the cell adhesion assay (b) and as such are not preferred compounds according to the invention.

Pharmaceutical Compositions

According to a further aspect of the invention there is provided a pharmaceutical composition which comprises a compound of the formula I, or a pharmaceutically acceptable salt or prodrug thereof, as defined hereinbefore in association with a pharmaceutically acceptable diluent or carrier.

The compositions of the invention may be in a form suitable for oral use (for example as tablets, lozenges, hard or soft capsules, aqueous or oily suspensions, emulsions, dispersible powders or granules, syrups or elixirs), for topical use (for example as creams, ointments, gels, or aqueous or oily solutions or suspensions), for administration by inhalation (for example as a finely divided powder or a liquid aerosol), for administration by insufflation (for example as a finely divided powder) or for parenteral administration (for example as a sterile aqueous or oily solution for intravenous, subcutaneous, intramuscular or intramuscular dosing or as a suppository for rectal dosing).

The compositions of the invention may be obtained by conventional procedures using conventional pharmaceutical excipients, well known in the art. Thus, compositions intended for oral use may contain, for example, one or more colouring, sweetening, flavouring and/or preservative agents.

An effective amount of a compound of the present invention for use in therapy of infection is an amount sufficient to symptomatically relieve in a warm-blooded animal, particularly a human the symptoms of infection, to slow the progression of infection, or to reduce in patients with symptoms of infection the risk of getting worse.

The amount of active ingredient that is combined with one or more excipients to produce a single dosage form will necessarily vary depending upon the host treated and the particular route of administration. For example, a formulation intended for oral administration to humans will generally contain, for example, from 0.5 mg to 0.5 g of active agent (more suitably from 0.5 to 100 mg, for example from 1 to 30 mg) compounded with an appropriate and convenient amount of excipients which may vary from about 5 to about 98 percent by weight of the total composition.

The size of the dose for therapeutic or prophylactic purposes of a compound of the formula I will naturally vary according to the nature and severity of the conditions, the age and sex of the animal or patient and the route of administration, according to well known principles of medicine.

In using a compound of the formula I for therapeutic or prophylactic purposes it will generally be administered so that a daily dose in the range, for example, 0.1 mg/kg to 75 mg/kg body weight is received, given if required in divided doses. In general lower doses will be administered when a parenteral route is employed. Thus, for example, for intravenous administration, a dose in the range, for example, 0.1 mg/kg to 30 mg/kg body weight will generally be used. Similarly, for administration by inhalation, a dose in the range, for example, 0.05 mg/kg to 25 mg/kg body weight will be used. Oral administration is however preferred, particularly in tablet form. Typically, unit dosage forms will contain about 0.5 mg to 0.5 g of a compound of this invention.

The compounds of the present invention are expected to possess, amongst others, anti-angiogenic properties such as anti-cancer properties that are believed to arise from the inhibition of a5b 1 function, particularly the compounds according to the invention are thought to be a5b1 antagonists. Furthermore, the compounds according to the present invention may possess substantially better potency against the a5b1 integrin, than against other integrins such as ανβ3, αiibβ3 or α4β1. Such compounds possess sufficient potency against the a5b1 integrin that they may be used in an amount sufficient to inhibit a5b1 function whilst demonstrating little, or significantly lower, activity against other integrins, such as those mentioned above. Such compounds are likely to be useful as selective a5b 1 antagonists and are likely to be useful for the effective treatment of, for example a5b 1 driven tumours.

Accordingly, the compounds of the present invention are expected to be useful in the treatment of diseases or medical conditions mediated alone or in part by a5b 1 integrin, i.e. the compounds may be used to produce an a5b1 antagonistic effect in a warm-blooded animal in need of such treatment. Thus the compounds of the present invention provide a method for the treatment of malignant cells characterised by inhibition of a5b 1 function. Particularly the compounds of the invention may be used to produce anti-angiogenic and/or an anti-proliferative and/or anti-invasive effect mediated alone or in part by the inhibition of a5b 1 function. Particularly, the compounds of the present invention are expected to be useful in the prevention or treatment of those tumours that are sensitive to inhibition of a5b 1 function that are involved in for example, angiogenesis, proliferation and the signal transduction steps which drive proliferation, invasion and particularly angiogenesis of these tumour cells. Accordingly the compounds of the present invention may be useful in the treatment of hyperproliferative disorders, including psoriasis, benign prostatic hyperplasia (BPH), atherosclerosis and restenosis and/or cancer by providing an anti-proliferative effect, particularly in the treatment of a5b 1 sensitive cancers. Such benign or malignant tumours may affect any tissue and include non-solid tumours such as leukaemia, multiple myeloma or lymphoma, and also solid tumours, for example bile duct, bone, bladder, brain/CNS, breast, colorectal, endometrial, gastric, head and neck, hepatic, lung, neuronal, oesophageal, ovarian, pancreatic, prostate, renal, skin, testicular, thyroid, uterine and vulval cancers. The compounds of the invention are expected to be useful in the treatment of pathogenic angiogenesis (pathological angiogenesis), for example in the treatment of cancers as hereinbefore described and other diseases in which inappropriate, or pathogenic angiogenesis occurs. By inappropriate, pathogenic or pathological angiogenesis is meant undesirable angiogenesis that results in an undesirable medical condition or disease such as age-related macular degeneration (AMD) or cancers involving a solid tumour. The compounds of the invention may also be useful in the treatment or prophylaxis of other conditions in which a5b1 is implicated, for example thrombosis, cardiac infarction, coronary heart diseases, arteriosclerosis, tumours, osteoporosis, inflammations such as psoriasis, gingivitis, osteoarthritis, rheumatoid arthritis, irritable bowel syndrome, ulcerative colitis or Crohn's disease, or infections.

In another aspect of the present invention there is provided a compound of formula I, or a pharmaceutically acceptable salt or prodrug thereof, as defined hereinbefore for use as a medicament.

In another embodiment the present invention provides the use of a compound of formula I or a pharmaceutically acceptable salt or prodrug thereof in the preparation of a medicament.

In another embodiment the present invention provides a compound of formula I or a pharmaceutically acceptable salt or prodrug thereof for use in the treatment or prophylaxis of a cancer, for example a cancer involving a solid tumour.

In another embodiment the present invention provides a compound of formula I or a pharmaceutically acceptable salt or prodrug thereof for use in the treatment or prophylaxis of neoplastic disease such as carcinoma of the breast, ovary, lung (including small cell lung cancer, non-small cell lung cancer and bronchioalveolar cancer), colon, rectum, prostate, bile duct, bone, bladder, head and neck, kidney, liver, gastrointestinal tissue, oesophagus, pancreas, skin, testes, thyroid, uterus, cervix, vulva or other tissues, as well as leukemias and lymphomas including CLL and CML, tumors of the central and peripheral nervous system, and other tumor types such as melanoma, multiple myeloma, fibrosarcoma and osteosarcoma, and malignant brain tumors.

In still another embodiment the present invention provides a compound of formula I or a pharmaceutically acceptable salt or prodrug thereof for use in the treatment or prophylaxis of pathologically angiogenic diseases, thrombosis, cardiac infarction, coronary heart diseases, arteriosclerosis, tumours, osteoporosis, inflammations or infections.

In another embodiment the present invention provides a compound of formula I or a pharmaceutically acceptable salt or prodrug thereof for use in the inhibition of a5b 1 function.

In another embodiment the present invention provides a compound of formula I or a pharmaceutically acceptable salt or prodrug thereof for use as an antiangiogenic agent in the treatment of a solid tumour.

In another embodiment the present invention provides the use of a compound of formula I or a pharmaceutically acceptable salt or prodrug thereof in the preparation of a medicament for the treatment or prophylaxis of a cancer, for example a cancer involving a solid tumour.

In another embodiment the present invention provides the use of a compound of formula I or a pharmaceutically acceptable salt or prodrug thereof in the preparation of a medicament for the treatment or prophylaxis of neoplastic disease such as carcinoma of the breast, ovary, lung (including small cell lung cancer, non-small cell lung cancer and bronchioalveolar cancer), colon, rectum, prostate, bile duct, bone, bladder, head and neck, kidney, liver, gastrointestinal tissue, oesophagus, pancreas, skin, testes, thyroid, uterus, cervix, vulva or other tissues, as well as leukemias and lymphomas including CLL and CML, tumors of the central and peripheral nervous system, and other tumor types such as melanoma, multiple myeloma, fibrosarcoma and osteosarcoma, and malignant brain tumors.

In still another embodiment the present invention provides the use of a compound of formula I or a pharmaceutically acceptable salt or prodrug thereof in the preparation of a medicament for the treatment or prophylaxis of pathologically angiogenic diseases, thrombosis, cardiac infarction, coronary heart diseases, arteriosclerosis, tumours, osteoporosis, inflammations or infections.

In another embodiment the present invention provides the use of a compound of formula I or a pharmaceutically acceptable salt or prodrug thereof in the preparation of a medicament for use in the inhibition of a5b 1 function.

In another embodiment the present invention provides the use of a compound of formula I or a pharmaceutically acceptable salt or prodrug thereof in the manufacture of a medicament for use as an antiangiogenic agent in the treatment of a solid tumour.

In a further aspect of the invention there is provided a pharmaceutical composition which comprises a compound of the formula I, or a pharmaceutically acceptable salt or prodrug thereof, as defined herein before in association with a pharmaceutically-acceptable diluent or carrier for use in the production of an a5b1 antagonistic effect in a warm-blooded animal such as man.

In a further aspect of the invention there is provided a pharmaceutical composition which comprises a compound of the formula I, or a pharmaceutically acceptable salt or prodrug thereof, as defined herein before in association with a pharmaceutically acceptable diluent or carrier for use in the production of an anti-cancer effect in a warm-blooded animal such as man.

In a further aspect of the invention there is provided a pharmaceutical composition which comprises a compound of the formula I, or a pharmaceutically acceptable salt or prodrug thereof, as defined herein before in association with a pharmaceutically acceptable diluent or carrier for use as an antiangiogenic agent in the treatment of a solid tumour.

In a further aspect of the invention there is provided a pharmaceutical composition which comprises a compound of the formula I, or a pharmaceutically acceptable salt or prodrug thereof, as defined herein before in association with a pharmaceutically-acceptable diluent or carrier for use in the treatment or prophylaxis of pathologically angiogenic diseases, thrombosis, cardiac infarction, coronary heart diseases, arteriosclerosis, tumours, osteoporosis, inflammations or infections.

In another embodiment the present invention provides a method of inhibiting pathological angiogenesis in a human or animal comprising administering to said human or animal a therapeutically effective amount of a compound of formula I or a pharmaceutically acceptable salt or prodrug thereof.

In a further embodiment the present invention provides a method of inhibiting a5b 1 function comprising administering to an animal or human in need of said inhibiting a therapeutically effective amount of a compound of formula I, or a pharmaceutically acceptable salt or prodrug thereof.

In a further embodiment the present invention provides a method of prophylaxis or treatment of a disease mediated in part or alone by a5b1 comprising administering to an animal or human in need of said prophylaxis or treatment a therapeutically effective amount of a compound of formula I, or a pharmaceutically acceptable salt or prodrug thereof.

In another embodiment the present invention provides a method of treatment of a human or animal suffering from cancer comprising administering to said human or animal a therapeutically effective amount of a compound of formula I, or a pharmaceutically acceptable salt or prodrug thereof.

In further embodiment the present invention provides a method of prophylaxis or treatment of cancer comprising administering to a human or animal in need of such prophylaxis or treatment a therapeutically effective amount of a compound of formula I, or a pharmaceutically acceptable salt or prodrug thereof.

In another embodiment the present invention provides a method of prophylaxis or treatment of a human or animal suffering from a neoplastic disease such as carcinoma of the breast, ovary, lung (including small cell lung cancer, non-small cell lung cancer and bronchioalveolar cancer), colon, rectum, prostate, bile duct, bone, bladder, head and neck, kidney, liver, gastrointestinal tissue, oesophagus, pancreas, skin, testes, thyroid, uterus, cervix, vulva or other tissues, as well as leukemias and lymphomas including CLL and CML, tumors of the central and peripheral nervous system, and other tumor types such as melanoma, multiple myeloma, fibrosarcoma and osteosarcoma, and malignant brain tumors, comprising administering to said human or animal a therapeutically effective amount of a compound of formula I, or a pharmaceutically acceptable salt or prodrug thereof.

In another embodiment the present invention provides a method of prophylaxis or treatment of a pathologically angiogenic disease, thrombosis, cardiac infarction, coronary heart diseases, arteriosclerosis, tumours, osteoporosis, inflammation or infection in a human or animal in need of such prophylaxis or treatment comprising administering to said human or animal a therapeutically effective amount of a compound of formula I or a pharmaceutically acceptable salt thereof.

Combination Therapies

The anti-cancer treatment defined hereinbefore may be applied as a sole therapy or may involve, in addition to the compound of the invention, conventional surgery or radiotherapy or chemotherapy. Such chemotherapy may include one or more of the following categories of anti-tumour agents:—

(i) antiproliferative/antineoplastic drugs and combinations thereof, as used in medical oncology, such as alkylating agents (for example cis-platin, oxaliplatin, carboplatin, cyclophosphamide, nitrogen mustard, melphalan, chlorambucil, busulfan, temozolamide and nitrosoureas); antimetabolites (for example gemcitabine and antifolates such as fluoropyrimidines like 5-fluorouracil and tegafur, raltitrexed, methotrexate, cytosine arabinoside, and hydroxyurea); antitumour antibiotics (for example anthracyclines like adriamycin, bleomycin, doxorubicin, daunomycin, epirubicin, idarubicin, mitomycin-C, dactinomycin and mithramycin); antimitotic agents (for example vinca alkaloids like vincristine, vinblastine, vindesine and vinorelbine and taxoids like taxol and taxotere and polokinase inhibitors); and topoisomerase inhibitors (for example epipodophyllotoxins like etoposide and teniposide, amsacrine, topotecan and camptothecin);

(ii) cytostatic agents such as antioestrogens (for example tamoxifen, fulvestrant, toremifene, raloxifene, droloxifene and iodoxyfene), antiandrogens (for example bicalutamide, flutamide, nilutamide and cyproterone acetate), LHRH antagonists or LHRH agonists (for example goserelin, leuprorelin and buserelin), progestogens (for example megestrol acetate), aromatase inhibitors (for example as anastrozole, letrozole, vorazole and exemestane) and inhibitors of 5α-reductase such as finasteride;

(iii) anti-invasion agents (for example c-Src kinase family inhibitors like 4-(6-chloro-2,3-methylenedioxyanilino)-7-[2-(4-methylpiperazin-1-yl)ethoxy]-5-tetrahydropyran-4-yloxyquinazoline (AZD0530; International Patent Application WO 01/94341) and N-(2-chloro-6-methylphenyl)-2-{6-[4-(2-hydroxyethyl)piperazin-1-yl]-2-methylpyrimidin-4-ylamino}thiazole-5-carboxamide (dasatinib, BMS-354825; J. Med. Chem., 2004, 47, 6658-6661), and metalloproteinase inhibitors like marimastat, inhibitors of urokinase plasminogen activator receptor function or antibodies to Heparanase);

(iv) inhibitors of growth factor function: for example such inhibitors include growth factor antibodies and growth factor receptor antibodies (for example the anti-erbB2 antibody trastuzumab [Herceptin™], the anti-EGFR antibody panitumumab, the anti-erbB1 antibody cetuximab [Erbitux, C225] and any growth factor or growth factor receptor antibodies disclosed by Stern et al. Critical reviews in oncology/haematology, 2005, Vol. 54, pp 11-29); such inhibitors also include tyrosine kinase inhibitors, for example inhibitors of the epidermal growth factor family (for example EGFR family tyrosine kinase inhibitors such as

N-(3-chloro-4-fluorophenyl)-7-methoxy-6-(3-morpholinopropoxy)quinazolin-4-amine (gefitinib, ZD 1839), N-(3-ethynylphenyl)-6,7-bis(2-methoxyethoxy)quinazolin-4-amine (erlotinib, OSI-774) and 6-acrylamido-N-(3-chloro-4-fluorophenyl)-7-(3-morpholinopropoxy)-quinazolin-4-amine (CI 1033), erbB2 tyrosine kinase inhibitors such as lapatinib, inhibitors of the hepatocyte growth factor family, inhibitors of the platelet-derived growth factor family such as imatinib, inhibitors of serine/threonine kinases (for example Ras/Raf signalling inhibitors such as farnesyl transferase inhibitors, for example sorafenib (BAY 43-9006)), inhibitors of cell signalling through MEK and/or AKT kinases, inhibitors of the hepatocyte growth factor family, c-kit inhibitors, abl kinase inhibitors, IGF receptor (insulin-like growth factor) kinase inhibitors; aurora kinase inhibitors (for example AZD1152, PH739358, VX-680, MLN8054, R763, MP235, MP529, VX-528 AND AX39459) and cyclin dependent kinase inhibitors such as CDK2 and/or CDK4 inhibitors;

(v) antiangiogenic agents such as those which inhibit the effects of vascular endothelial growth factor, [for example the anti-vascular endothelial cell growth factor antibody bevacizumab (Avastin™) and VEGF receptor tyrosine kinase inhibitors such as 4-(4-bromo-2-fluoroanilino)-6-methoxy-7-(1-methylpiperidin-4-ylmethoxy)quinazoline (ZD6474; Example 2 within WO 01/32651), 4-(4-fluoro-2-methylindol-5-yloxy)-6-methoxy-7-(3-pyrrolidin-1-ylpropoxy)quinazoline (AZD2171; Example 240 within WO 00/47212), vatalanib (PTK787; WO 98/35985) and SU11248 (sunitinib; WO 01/60814), compounds such as those disclosed in International Patent Applications WO97/22596, WO 97/30035, WO 97/32856 and WO 98/13354 and compounds that work by other mechanisms (for example linomide, inhibitors of integrin Uvp3 function and angiostatin)];

(vi) vascular damaging agents such as Combretastatin A4 and compounds disclosed in International Patent Applications WO 99/02166, WO 00/40529, WO 00/41669, WO 01/92224, WO 02/04434 and WO 02/08213;

(vii) antisense therapies, for example those which are directed to the targets listed above, such as ISIS 2503, an anti-ras antisense;

(viii) gene therapy approaches, including for example approaches to replace aberrant genes such as aberrant p53 or aberrant BRCA1 or BRCA2, GDEPT (gene-directed enzyme pro-drug therapy) approaches such as those using cytosine deaminase, thymidine kinase or a bacterial nitroreductase enzyme and approaches to increase patient tolerance to chemotherapy or radiotherapy such as multi-drug resistance gene therapy; and

(ix) immunotherapy approaches, including for example ex-vivo and in-vivo approaches to increase the immunogenicity of patient tumour cells, such as transfection with cytokines such as interleukin 2, interleukin 4 or granulocyte-macrophage colony stimulating factor, approaches to decrease T-cell anergy, approaches using transfected immune cells such as cytokine-transfected dendritic cells, approaches using cytokine-transfected tumour cell lines and approaches using anti-idiotypic antibodies.

Such conjoint treatment may be achieved by way of the simultaneous, sequential or separate dosing of the individual components of the treatment. Such combination products employ the compounds of this invention within the dosage range described hereinbefore and the other pharmaceutically-active agent within its approved dosage range.

Accordingly a further aspect of the invention provides a method of treatment of a cancer comprising administering to a human or animal in need of such treatment a therapeutically effective amount of:

(a) a compound of formula I, or a pharmaceutically acceptable salt or prodrug thereof, as defined hereinbefore; and

(b) an additional chemotherapeutic agent.

Suitable additional chemotherapeutic agents are as hereinbefore defined in relation to combination therapies, for example one or more agents selected from selected from:

(i) an antiangiogenic agent;

(ii) a cytostatic agent;

(iii) an antiproliferative agent

(iv) an antineoplastic agent;

(v) an anti-invasion agent;

(vi) an inhibitor of growth factor function; and

(vii) a vascular damaging agent.

### EXAMPLES

The invention will now be illustrated in the following Examples in which, generally:

(i) operations were carried out at ambient temperature, i.e. in the range 17 to 25° C. and under an atmosphere of an inert gas such as nitrogen or argon unless otherwise stated;

(ii) in general, the course of reactions was followed by thin layer chromatography (TLC) and/or analytical high pressure liquid chromatography (HPLC); the reaction times that are given are not necessarily the minimum attainable;

(iii) when necessary, organic solutions were dried over anhydrous magnesium sulfate, work-up procedures were carried out using traditional layer separating techniques, evaporations were carried out either by rotary evaporation in vacuo or in a Genevac HT-4/EZ-2.

(iv) yields, where present, are not necessarily the maximum attainable, and when necessary, reactions were repeated if a larger amount of the reaction product was required;

(v) in general, the structures of the end-products of the formula I were confirmed by nuclear magnetic resonance (NMR) and/or mass spectral techniques; electrospray mass spectral data were obtained using a Waters ZMD or Waters ZQ LC/mass spectrometer acquiring both positive and negative ion data, generally, only ions relating to the parent structure are reported; proton NMR chemical shift values were measured on the delta scale using a Bruker Advance operating at 500 MHz. The following abbreviations have been used: s, singlet; d, doublet; t, triplet; q, quartet; m, multiplet; br, broad;

(vi) unless stated otherwise compounds containing an asymmetric carbon and/or sulfur atom were not resolved;

(vii) intermediates were not necessarily fully purified but their structures and purity were assessed by TLC, analytical HPLC, infra-red (IR) and/or NMI analysis;

(viii) unless otherwise stated, column chromatography (by the flash procedure) and medium pressure liquid chromatography (MPLC) were performed on Merck Kieselgel silica (Art. 9385);

(ix) the following analytical HPLC methods were used; in general, reversed-phase silica was used with a flow rate of about 1 ml per minute and detection was by Electrospray Mass Spectrometry and by UV absorbance at a wavelength of 254 nm;

(x) where certain compounds were obtained as an acid-addition salt, for example a mono-hydrochloride salt or a di-hydrochloride salt, the stoichiometry of the salt was based on the number and nature of the basic groups in the compound, the exact stoichiometry of the salt was generally not determined, for example by means of elemental analysis data;

(xi) the following abbreviations have been used:—
AcOH: acetic acid
ADDP: 1,1′-(azadicarbonyl)dipiperidine
BOC₂O: di-ter-butyl dicarbonate
BBN 9-borabicyclo[3.3.1]nonane
BOC tert-butoxycarbonyl
mCPBA: 3-chloroperbenzoic acid
DCC: N,N′-dicyclohexylcarbodiimide
DMAP: 4-dimethylaminopyridine
DMF: N,N-dimethylformamide
DMSO: dimethylsulfoxide
DMA: N-dimethylacetamide
DCM: dichloromethane
DIPEA: N,N′-diisopropylethylamine
DTAD: di-tert-butyl azodicarboxylate
dppf: 1,1′-bis(diphenylphosphino)ferrocene
dppp: 1,3-bis(diphenylphosphino)propane
EDCI: 1-(3-dimethylaminopropyl)-3-ethylcarbodiimide hydrochloride
EtOAc ethyl acetate
HATU: O-(7-Azabenzotriazol-1-yl)-N,N,N′,N′-Tetramethyluronium Hexafluoro-Phosphate
HOPO: 2-hydroxypyridine 1-oxide
NMP: N-methypyrrolidone
PTSA: paratoluenesulfonic acid
TBTU: O-benzotriazol-1-yl-N,N,N′,N′-tetramethyluronium tetrafluoroborate
TFA: trifluoroacetic acid
THF: tetrahydrofuran
THP: tetrahydropyran-2-yl

Preparative HPLC was performed on C18 reverse phase-silica, for example on a Waters ‘Xterra’ preparative reverse-phase column (5 microns silica, 19 mm diameter, 100 mm length) using decreasingly polar mixtures as eluent, for example decreasingly polar mixtures of water containing 1% acetic acid (acidic conditions) or (NH₄)₂CO₃ (4 g/l) (basic conditions) and acetonitrile;

### Example 1

The compound of Example 1.1 described below and the compounds shown in Table 1 were prepared using the following reaction scheme:

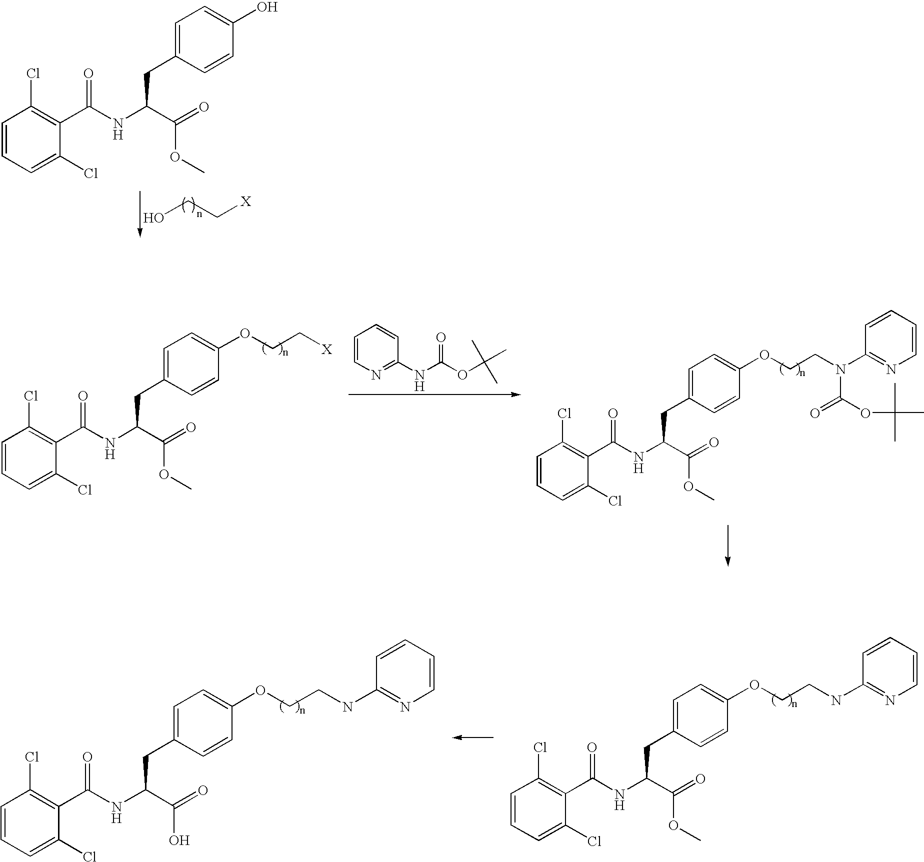

### Example 1.1

### N-(2,6-dichlorobenzoyl)-O-[2-(pyridin-2-ylamino)ethyl]-L-tyrosine

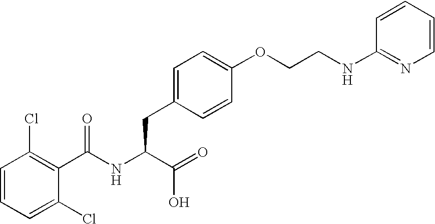

LiOH (40 mg) was added to a solution of methyl N-(2,6-dichlorobenzoyl)-O-[2-(pyridin-2-ylamino)ethyl]-L-tyrosinate (170 mg, 0.35 mmol) in a mixture DMA/H₂O (0.7/0.4 ml). After 4 hours at room temperature, the crude reaction mixture was purified by C18 reverse phase chromatography (basic conditions) to afford the title compound as a solid (68 mg, 41%); Mass Spectrum [M+H]⁺=474; ¹NMR (500 MHz): (DMSO-d6) 2.88 (1H, dd) 3.05 (1H, dd), 3.56-3.66 (2H, m), 4.01-4.11 (2H, m), 4.59 (1H, m), 6.48 (1H, dd), 6.53 (1H, d), 6.71 (1H, bs), 6.87 (2H, d), 7.20 (2H, d), 7.31-7.50 (4H, m), 7.98 (1H, d), 8.99 (1H, bs).

### Examples 1.2 and 1.3

### Process (a)

The procedure described above for Example 1.1 was repeated using the appropriate methyl ester precursor. Thus were obtained the compounds shown in Table 1:

TABLE 1

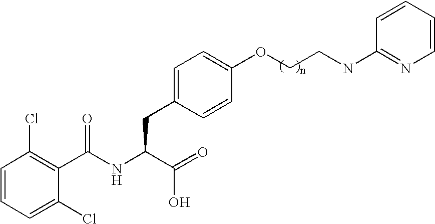

| | | | | Mass | ¹H NMR Data |
|---|---|---|---|---|---|
| Example | n | Name | Yield | Ion | (500 MHz) |
| 1.2 | 2 | N-(2,6- | 74% | 488 | (DMSO-d6) 1.91-1.99 (2H, m), |
| | | dichlorobenzoyl)- | | [M + H]⁻ | 2.91 (1H, dd) 3.05 (1H, dd), |
| | | O-[3-(pyridin-2- | | | 3.34-3.40 (2H, m) [partially |
| | | ylamino)propyl]- | | | hidden by H₂O], 4.00 (2H, t), |
| | | L-tyrosine | | | 4.40-4.48 (1H, m), 6.44 (1H, dd), |
| | | | | | 6.45 (1H, s), 6.54 (1H, t), 6.80 |
| | | | | | (2H, d), 7.17 (2H, d), 7.34 (1H, |
| | | | | | ddd), 7.38 (1H, dd), 7.42 (1H, s), |
| | | | | | 7.43 (1H, dd), 7.95 (1H, dd), |
| | | | | | 8.58 (1H, bs) |
| 1.3 | 3 | N-(2,6- | 44% | 502 | (DMSO-d6) 1.60-1.69 (2H, m), |
| | | dichlorobenzoyl)- | | [M + H]⁻ | 1.71-1.80 (2H, m), 2.91 (1H, dd) |
| | | O-[4-(pyridin-2- | | | 3.05 (1H, dd), 3.23-3.29 (2H, m), |
| | | ylamino)butyl]- | | | 3.94 (2H, t), 4.41 (1H, bs), 6.40- |
| | | L-tyrosine | | | 6.45 (2H, m), 6.48 (1H, t), 6.78 |
| | | | | | (2H, d), 7.16 (2H, d), 7.33 (1H, |
| | | | | | ddd), 7.38 (1H, dd), 7.42 (1H, s), |
| | | | | | 7.43 (1H, dd), 7.94 (1H, dd), 8.49 |
| | | | | | (1H, bs) |

The starting materials used in Examples 1.1 to 1.3 were prepared as follows:

Methyl L-tyrosinate hydrochloride (10 g) was suspended in DCM (200 ml) at 4° C. and triethylamine (13.2 ml) was added as a single portion. 2,6-dichlorobenzoyl chloride (6.18 ml) was added dropwise (T<9° C.) and the reaction stirred overnight at room temperature. The solution was washed with water and brine. Upon standing, a solid precipitate formed from the aqueous layer. The precipitate was filtered, washed with water, and dried to give methyl N-(2,6-dichlorobenzoyl)-L-tyrosinate as a white solid (4 g). The organic layer was dried and concentrated to give an off-white solid (12 g). The two solids were combined and triturated with a mixture of ethyl acetate/ether/iso-hexane, filtered, and dried to give methyl N-(2,6-dichlorobenzoyl)-L-tyrosinate as a white solid (12.5 g, 79%); ¹H NMR Spectrum: (DMSO-d6) 2.85 (1H, dd), 3.0 (1H, dd), 3.65 (3H, s), 4.65 (1H, m), 6.65 (2H, d), 7.05 (2H, d), 7.45 (3H, m), 9.10 (1H, d), 9.15 (1H, s); Mass Spectrum [M+H]⁺=368.

Triphenylphosphine (7.92 g, 29.9 mmol) and 2-bromoethanol (1.45 ml, 20.4 mmol) were added to a solution of methyl N-(2,6-dichlorobenzoyl)-L-tyrosinate (5 g, 13.6 mmol) in DCM (5 ml) at 0° C. DTAD was then added (6.26 g, 27.2 mmol). The reaction mixture was allowed to stir at room temperature overnight, and was then concentrated and purified by silica gel flash chromatography (0 to 40% ethyl acetate in petroleum ether) to give methyl O-(2-bromoethyl)-N-(2,6-dichlorobenzoyl)-L-tyrosinate as a white solid (4.3 g, 65%).

The procedure described above for the preparation of methyl O-(2-bromoethyl)-N-(2,6-dichlorobenzoyl)-L-tyrosinate was repeated using the appropriate bromo or chloro commercially available alcohol. Thus were obtained the other compounds described in Table 1.1:

TABLE 1.1

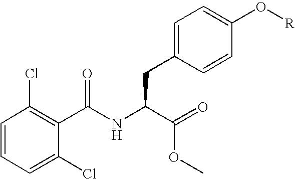

| | | | | Mass | ¹H NMR Data |
|---|---|---|---|---|---|
| Compound | R | Name | yield | Ion | (500 MHz) |
| 1.1a | 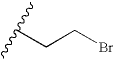 | methyl O-(2- bromoethyl)- N-(2,6- dichlorobenzoyl)- L-tyrosinate | 65% | 475 [M + H]⁺ | (DMSO-d6) 2.90 (1H, dd), 3.06 (1H, dd), 3.65 (3H, s), 3.78 (2H, t), 4.29 (2H, t), 4.67 (1H, ddd), 6.86 (2H, d), 7.19 (d, 2H), 7.40 (1H, dd), 7.44 (1H, s), 7.45 (dd, 1H), 9.18 (1H, d) |
| 1.2a | 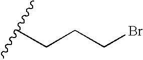 | methyl O-(3- bromopropyl)- N-(2,6- dichlorobenzoyl)- L-tyrosinate | 89% | 489 [M + H]⁺ | (DMSO-d6) 2.18-2.27 (2H, m), 2.89 (1H, dd), 3.05 (1H, dd), 3.64 (3H, s), 3.66 (2H, t), 4.05 (2H, t), 4.67 (1H, ddd), 6.87 (2H, d), 7.20 (d, 2H), 7.40 (1H, dd), 7.45 (1H, s), 7.46 (dd, 1H), 9.19 (1H, |
| # d) | | | | | |
| 1.3a | 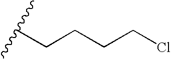 | methyl O-(4- chlorobutyl)- N-(2,6- dichlorobenzoyl)- L-tyrosinate | 81% | — | (DMSO-d6) 1.77-1.91 (4H, m), 2.88 (1H, dd), 3.04 (1H, dd), 3.64 (3H, s), 3.70 (2H, t), 3.96 (2H, t), 4.66 (1H, ddd), 6.84 (2H, d), 7.18 (2H, d), 7.40 (1H, dd), 7.45 (1H, s), 7.45 (dd, 1H), 9.18 (1H, d) |

Sodium hydride (60% in oil, 68 mg, 1.68 mmol) was added to a solution of tert-butyl pyridin-2-ylcarbamate (326 mg, 1.68 mmol) in DMA (3.5 ml) at 0° C. The temperature was raised to room temperature for 15 minutes, then methyl O-(2-bromoethyl)-N-(2,6-dichlorobenzoyl)-L-tyrosinate was added (400 mg, 0.84 mmol) and the reaction mixture stirred at 50° C. for 2 hours. A few drops of water were added to the reaction mixture, then solvents were evaporated. The residue was dissolved in DCM and was partially purified by silica gel flash chromatography (0 to 100% Ethyl acetate in petroleum ether) to give methyl O-{2-[(tert-butoxycarbonyl)(pyridin-2-yl)amino]ethyl}-N-(2,6-dichlorobenzoyl)-L-tyrosinate in mixture with impurities (440 mg). This was used in the next step without further purification.

The procedure described above for the preparation of methyl O-{2-[(tert-butoxycarbonyl)(pyridin-2-yl)amino]ethyl}-N-(2,6-dichlorobenzoyl)-L-tyrosinate was repeated using the appropriate bromo or chloro precursor. Thus were obtained the compounds described in Table 1.2:

TABLE 1.2

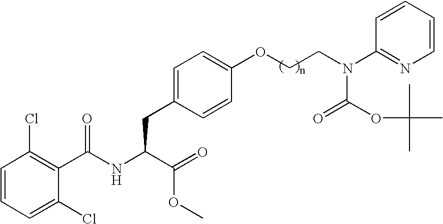

| | | | Mass | ¹H NMR Data |
|---|---|---|---|---|
| Compound | n | Name | Ion | (500 MHz) |
| 1.1b | 1 | Methyl O-{2-[(tert- | 588 | (DMSO-d6) 1.46 (9H, s), 2.87 |
| | | butoxycarbonyl) | [M + H]⁺ | (1H, dd), 3.03 (1H, dd), 3.64 |
| | | (pyridin-2- | | (3H, s), 4.12-4.17 (2H, m), 4.17- |
| | | yl)amino]ethyl}-N- | | 4.22 (2H, m), 4.65 (1H, ddd), |
| | | (2,6-dichlorobenzoyl)- | | 6.78 (2H, d), 7.12-7.18 (3H, m), |
| | | L-tyrosinate | | 7.40 (1H, dd), 7.44 (1H, s), 7.45 |
| | | | | (1H, dd), 7.56 (1H, d), 7.77 (1H, |
| | | | | ddd), 8.38 (1H, dd), 9.17 (1H, d) |
| 1.2b | 2 | methyl O-{3-[(tert | 600 | (DMSO-d6) 1.43 (9H, s), 1.94- |
| | | butoxycarbonyl) | [M − H]⁻ | 2.02 (2H, m), 2.88 (1H, dd), |
| | | (pyridin-2- | | 3.03 (1H, dd), 3.64 (3H, t), 3.94 |
| | | yl)amino]propyl}-N- | | (2H, t), 4.01 (2H, t), 4.65 (1H, |
| | | (2,6-dichlorobenzoyl)- | | ddd), 6.78 (2H, d), 7.13 (1H, |
| | | L-tyrosinate | | ddd), 7.16 (2H, d), 7.41 (1H, |
| | | | | dd), 7.44 (1H, s), 7.45 (1H, dd), |
| | | | | 7.57 (1H, d), 7.75 (1H, ddd), |
| | | | | 8.37 (1H, dd), 9.18 (1H, d) |
| 1.3b | 3 | methyl-O-{4-[(tert- | 616 | (DMSO-d6) 1.44 (9H, s), 1.65- |
| | | butoxycarbonyl) | [M + H]⁺ | 1.71 (4H, m), 2.88 (1H, dd), |
| | | pyridin-2-yl) | | 3.04 (1H, dd), 3.64 (3H, s), 3.86- |
| | | amino]butyl}- | | 3.94 (4H, m), 4.65 (1H, ddd), |
| | | N-(2,6- | | 6.79 (2H, d), 7.13 (1H, ddd), |
| | | dichlorobenzoyl)- | | 7.16 (2H, d), 7.40 (1H, dd), 7.44 |
| | | L-tyrosinate | | (1H, s), 7.45 (1H, dd), 7.53 (1H, |
| | | | | d), 7.75 (1H, ddd), 8.37 (1H, |
| | | | | dd), 9.17 (1H, d) |

TFA (2 ml) was added to a solution of methyl O-{2-[(tert-butoxycarbonyl)(pyridin-2-yl)amino]ethyl}-N-(2,6-dichlorobenzoyl)-L-tyrosinate (400 mg) in DCM (2 ml). The reaction mixture was stirred for 2 hours at room temperature, then solvent was removed by evaporation. The residue was dissolved in water and NaOH 2N was added dropwise until a white suspension appeared. Extraction with ethyl acetate and evaporation of the organic layer was followed by purification by C18 reverse phase chromatography (basic conditions) to afford methyl N-(2,6-dichlorobenzoyl)-O-[2-(pyridin-2-ylamino)ethyl]-L-tyrosinate (290 mg, 71% over 2 steps).

The procedure described above for the preparation of methyl N-(2,6-dichlorobenzoyl)-O-[2-(pyridin-2-ylamino)ethyl]-L-tyrosinate was repeated using the appropriate N-BOC precursor. Thus were obtained the compounds described in table 1.3 below:

TABLE 1.3

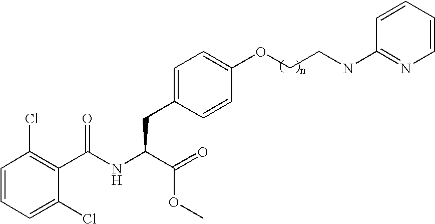

| | | | Yield | | |
|---|---|---|---|---|---|
| | | | over 2 | Mass | ¹H NMR Data |
| Compound | n | Name | steps | Ion | (500 MHz) |
| 1.1c | 1 | methyl N-(2,6- | 71% | 488 | (DMSO-d6) 2.88 (1H, dd), |
| | | dichlorobenzoyl)- | | [M + H]⁺ | 3.04 (1H, dd), 3.57-3.63 (2H, |
| | | O-[2-(pyridin-2- | | | m), 3.64 (3H, s), 4.05 (2H, t), |
| | | ylamino)ethyl]- | | | 4.66 (1H, ddd), 6.48 (1H, |
| | | L-tyrosinate | | | dd), 6.52 (1H, d), 6.70 (1H, |
| | | | | | t), 6.87 (2H, d), 7.18 (2H, d), |
| | | | | | 7.36 (1H, ddd), 7.40 (1H, |
| | | | | | dd), 7.44 (1H, s), 7.45 (1H, |
| | | | | | dd), 7.98 (1H, dd), 9.18 (1H, |
| | | | | | d) |
| 1.2c | 2 | methyl N-(2,6- | 29% | 502 | — |
| | | dichlorobenzoyl)- | | [M + H]⁺ | |
| | | O-[3-(pyridin-2- | | | |
| | | ylamino)propyl]- | | | |
| | | L-tyrosinate | | | |
| 1.3c | 3 | methyl N-(2,6- | 16% | 516 | |
| | | dichlorobenzoyl)- | | [M + H]⁺ | |
| | | O-[4-(pyridin-2- | | | |
| | | ylamino)butyl]- | | | |
| | | L-tyrosinate | | | |

### Example 2

### N-(2,6-dichlorobenzoyl)-O-(5,6,7,8-tetrahydro-1,8-naphthyridin-2-ylmethyl)-L-tyrosine

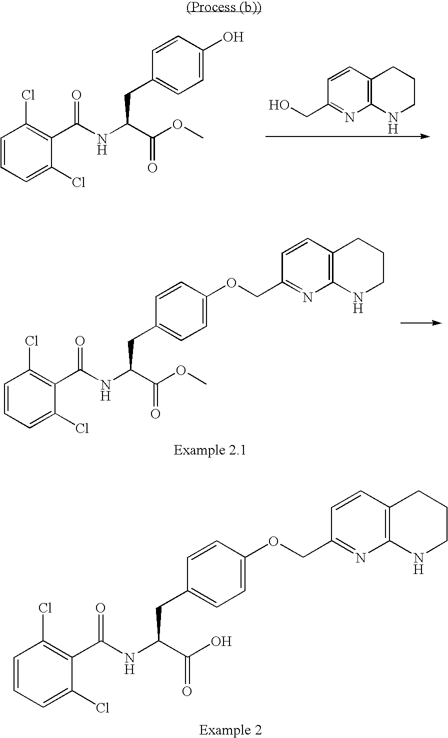

LiOH, H₂O (12 mg, 0.28 mmol) was added to a solution of methyl N-(2,6-dichlorobenzoyl)-O-(5,6,7,8-tetrahydro-1,8-naphthyridin-2-ylmethyl)-L-tyrosinate (71 mg, 0.14 mmol) in MeCN (2 ml). After 2 hours at room temperature the crude reaction mixture was concentrated, dissolved in DMF and purified by C18 reverse phase chromatography (basic conditions) to afford the title compound as a solid (40 mg, 58%); Mass Spectrum [M+H]⁺=500; ¹H NMR Spectrum (DMSO-d6): 1.73-1.82 (2H, m), 2.65 (2H, t), 2.87 (1H, dd), 3.05 (1H, dd), 2.23-2.31 (2H, m), 4.55 (1H, ddd), 4.83 (2H, s), 6.48 (1H, d), 6.50 (1H, bs), 6.85 (2H, d), 7.13 (1H, d), 7.18 (2H, d), 7.39 (1H, dd), 7.43 (1H, s), 7.44 (1H, dd), 8.87 (1H, d).

The methyl N-(2,6-dichlorobenzoyl)-O-(5,6,7,8-tetrahydro-1,8-naphthyridin-2-ylmethyl)-L-tyrosinate used as the starting material was prepared as follows:

Methyl N-(2,6-dichlorobenzoyl)-L-tyrosinate (200 mg, 0.54 mmol) was added to a solution of triphenylphosphine (212 mg, 0.81 mmol) and DTAD (187 mg, 0.81 mmol) in DCM (2 ml). Then 5,6,7,8-tetrahydro-1,8-naphthyridin-2-ylmethanol (107 mg, 0.65 mmol) in DCM (2 ml) was added dropwise under argon. The reaction mixture was allowed to stir at room temperature for 2 hours, then was directly purified by silica gel flash chromatography (10 to 50% Ethyl acetate in DCM) to afford methyl N-(2,6-dichlorobenzoyl)-O-(5,6,7,8-tetrahydro-1,8-naphthyridin-2-ylmethyl)-L-tyrosinate as a white solid (71 mg, 25%); Mass Spectrum [M+H]⁺=514; ¹H NMR Spectrum (DMSO-d6) 1.72-1.81 (2H, m), 2.61-2.67 (2H, m), 2.87 (1H, dd), 3.04 (1H, dd), 3.22-3.29 (2H, m), 3.64 (3H, s), 4.65 (1H, ddd), 4.83 (2H, s), 6.47 (1H, d), 6.49 (1H, bs), 6.86 (2H, d), 7.12 (1H, d), 7.17 (2H, d), 7.40 (1H, dd), 7.43-7.47 (2H, m), 9.17 (1H, d).

### Example 3

### N-(2,6-dichlorobenzoyl)-O-[2-(5,6,7,8-tetrahydro-1,8-naphthyridin-2-yl)ethyl]-L-tyrosine

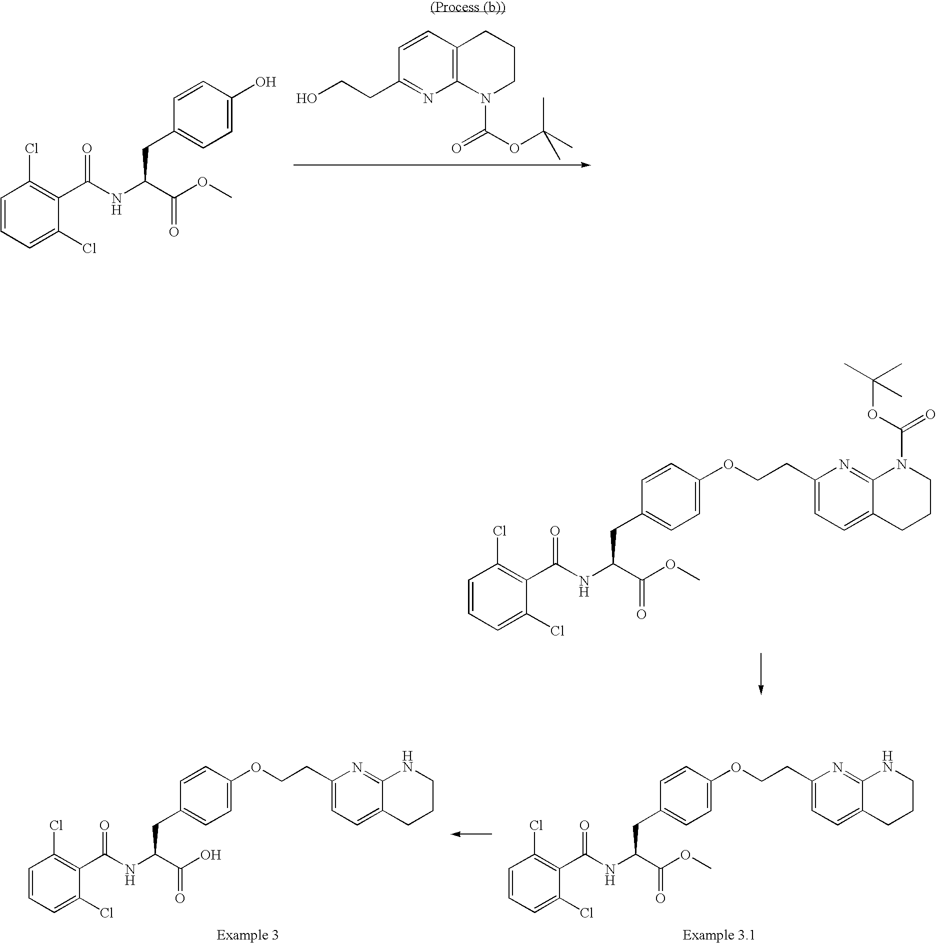

LiOH, H₂O (8 mg, 0.18 mmol) was added to a solution of methyl N-(2,6-dichlorobenzoyl)-O-[2-(5,6,7,8-tetrahydro-1,8-naphthyridin-2-yl)ethyl]-L-tyrosinate (49 mg, 0.09 mmol) in DMA (1 ml). After 1.5 hours at room temperature, the crude reaction mixture was purified by C18 reverse phase chromatography (basic conditions) to afford the title compound as a white solid (36 mg, 75%); Mass Spectrum [M+H]⁺=514; ¹H NMR Spectrum: (DMSO-d6) 1.71-1.81 (2H, m), 2.62 (2H, t), 2.83-2.92 (3H, m), 3.06 (1H, dd), 3.21-3.28 (2H, m), 4.19 (2H, t), 4.53-4.60 (1H, m), 6.36 (1H, d), 6.40 (1H, bs), 6.81 (2H, d), 7.06 (1H, d), 7.18 (2H, d), 7.39 (1H, dd), 7.42-7.46 (2H, m), 8.86 (1H, d).

The methyl N-(2,6-dichlorobenzoyl)-O-[2-(5,6,7,8-tetrahydro-1,8-naphthyridin-2-yl)ethyl]-L-tyrosinate used as the starting material was prepared as follows:

Methyl N-(2,6-dichlorobenzoyl)-L-tyrosinate (180 mg, 0.49 mmol) and tert-butyl 7-(2-hydroxyethyl)-3,4-dihydro-1,8-naphthyridine-1(2H)-carboxylate (150 mg, 0.54 mmol) in DCM (2 ml) were added dropwise to a solution of triphenylphosphine (191 mg, 0.73 mmol) and DTAD (168 mg, 0.73 mmol) in DCM (2 ml) at 0° C. The reaction mixture was allowed to stir at room temperature for 4 hours and then purified by silica gel flash chromatography (20 to 50% ethyl acetate in petroleum ether) to afford tert-butyl 7-[2-(4-{(2S)-2-[(2,6-dichlorobenzoyl)amino]-3-methoxy-3-oxopropyl}phenoxy)ethyl]-3,4-dihydro-1,8-naphthyridine-1(2H)-carboxylate as a white foam (80 mg, 26%); Mass Spectrum [M+H]⁺=628; ¹H NMR Spectrum: (DMSO-d6) 1.43 (9H, s), 1.78-1.86 (2H, m), 2.70 (2H, t), 2.88 (1H, dd), 3.04 (1H, dd), 3.06 (2H, t), 3.63 (2H, t), 3.64 (3H, s), 3.49 (2H, t), 4.66 (1H, ddd), 6.83 (2H, d), 6.99 (1H, d), 7.17 (2H, d), 7.40 (1H, dd), 7.43-7.47 (3H, m), 9.17 (1H, d).

TFA (1 ml) was added to a solution of tert-butyl 7-[2-(4-{(2S)-2-[(2,6-dichlorobenzoyl)amino]-3-methoxy-3-oxopropyl}phenoxy)ethyl]-3,4-dihydro-1,8-naphthyridine-1(2H)-carboxylate (80 mg) in DCM (1 ml). The reaction mixture was stirred 2 hours at room temperature, then solvent was removed. The residue was dissolved in DCM and NH₃/MeOH 7N was added. Evaporation and dissolution in DCM was followed by a purification by silica gel flash chromatography (DCM/MeOH 95/5) to afford Example 3.1: methyl N-(2,6-dichlorobenzoyl)-O-[2-(5,6,7,8-tetrahydro-1,8-naphthyridin-2-yl)ethyl]-L-tyrosinate as a white solid (49 mg, 73%); Mass Spectrum [M+H]⁺=528; ¹H NMR Spectrum: (DMSO-d6) 1.71-1.80 (2H, m), 2.61 (2H, t), 2.86 (2H, t), 2.88 (1H, dd), 3.04 (1H, dd), 3.20-3.27 (2H, m), 3.64 (3H, s), 4.19 (2H, t), 4.66 (1H, ddd), 6.33 (1H, bs), 6.35 (1H, d), 6.82 (2H, d), 7.05 (1H, d), 7.17 (2H, d), 7.40 (1H, dd), 7.43-7.48 (2H, m), 9.18 (1H, d).

### Example 4

### N-(2,6-dichlorobenzoyl)-O-{[6-(methylamino)pyridin-2-yl]methyl}-L-tyrosine

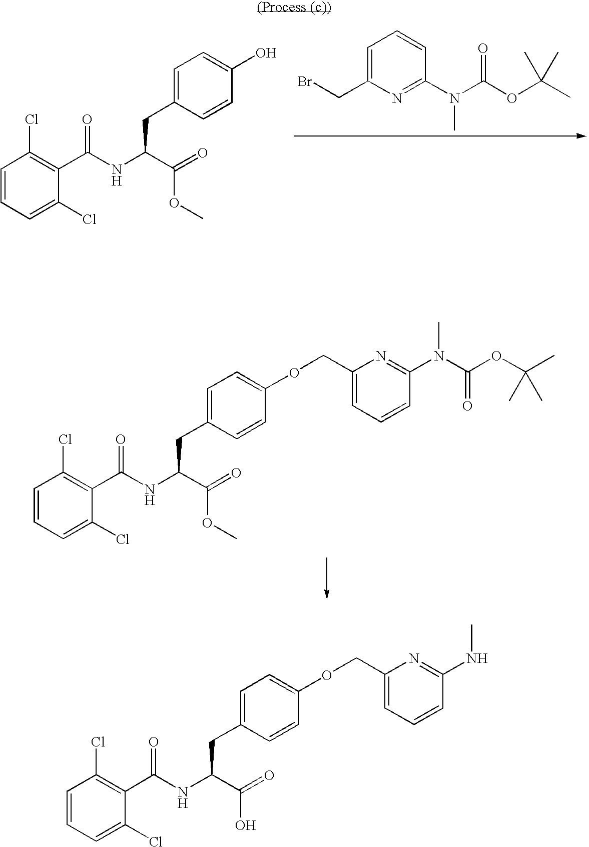

A solution of HCl 4M in dioxane (0.1 ml) was added to a solution of methyl O-({6-[(tert-butoxycarbonyl)(methyl)amino]pyridin-2-yl}methyl)-N-(2,6-dichlorobenzoyl)-L-tyrosinate (80 mg, 0.14 mmol) in MeOH (1 ml). The reaction mixture was heated at 45° C. for 4 hours to reach completion. Solvents were evaporated, the residue was dissolved in MeOH (1 ml) and an aqueous solution of LiOH 2N (0.27 ml) was added. After 0.5 hour the crude reaction was directly purified by C18 reverse phase chromatography (basic conditions) to afford the title compound as a white solid (35 mg, 55%); ¹H NMR Spectrum (DMSO-d6) 2.76 (3H, d), 2.87 (1H, dd), 3.06 (1H, dd), 4.57 (1H, dd), 4.91 (2H, s), 6.35 (1H, d), 6.50 (1H, q), 6.55 (1H, d), 6.88 (2H, d), 7.19 (2H, d), 7.33-7.47 (4H, m), 8.93 (1H, bs); Mass Spectrum [M+H]⁺=474.

The methyl O-({6-[(tert-butoxycarbonyl)(methyl)amino]pyridin-2-yl}methyl)-N-(2,6-dichlorobenzoyl)-L-tyrosinate used as the starting material was prepared as follows:

N-Bromo succinimide (178 mg, 1.0 mmol) and catalytic benzoyl peroxide (1 mg, 0.005 mmol) were added to a solution of tert-butyl methyl(6-methylpyridin-2-yl)carbamate (222 mg, 1.0 mmol) in CCl₄ at room temperature. The reaction was heated at 50° C. overnight. After evaporation of solvent, the crude mixture was purified by silica gel flash chromatography (0 to 5% diethyl ether in petroleum ether) to afford 2 different brominated compounds, the second one in order of elution being the expected tert-butyl [6-(bromomethyl)pyridin-2-yl]methylcarbamate, obtained as a colorless oil (50 mg, 17%); ¹H NMR Spectrum (CDCl3) 1.52 (9H, s), 3.40 (3H, s), 4.48 (2H, s), 7.11 (1H, d), 7.61 (2H, m).

K₂CO₃ (25 mg, 0.18 mmol), then tert-butyl [6-(bromomethyl)pyridin-2-yl]methylcarbamate (48 mg, 0.16 mmol) were added to a solution of methyl N-(2,6-dichlorobenzoyl)-L-tyrosinate (60 mg, 0.16 mmol) in DMF (1 ml). The reaction mixture was allowed to stir at room temperature for 6 hours, then solvent was removed and the crude mixture was purified by silica gel flash chromatography (0 to 40% Diethyl ether in petroleum ether) to afford methyl O-({6-[(tert-butoxycarbonyl) (methyl)amino]pyridin-2-yl}methyl)-N-(2,6-dichlorobenzoyl)-L-tyrosinate as a white foam (80 mg, 83%); ¹H NMR Spectrum (DMSO-d6) 1.47 (9H, s), 2.88 (1H, dd), 3.06 (1H, dd), 3.29 (3H, s), 3.64 (3H, s), 4.67 (1H, ddd), 5.09 (2H, s), 6.94 (2H, d), 7.20 (2H, d), 7.21 (1H, d), 7.40 (1H, dd), 7.44 (1H, s), 7.45 (1H, dd), 7.55 (1H, d), 7.78 (1H, dd), 9.17 (1H, d); Mass Spectrum [M+H]⁺=588.

### Example 5

The compounds of Example 5 described below and in Table 2 were prepared using the following reaction scheme:

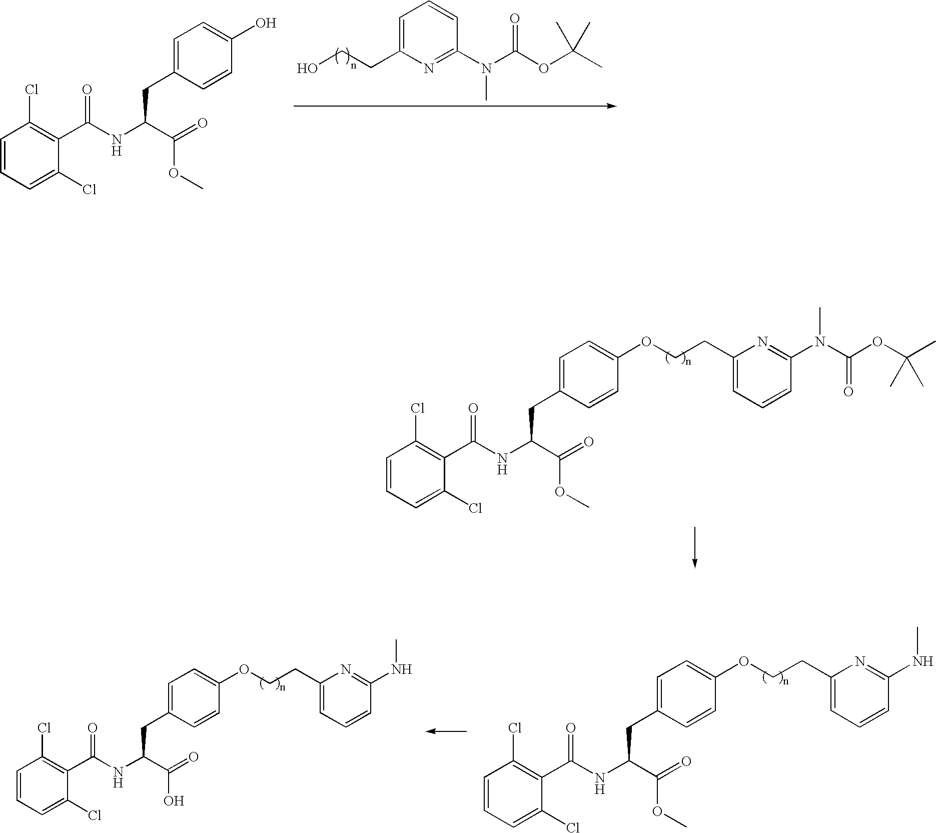

### Example 5.1

### N-(2,6-dichlorobenzoyl)-O-{2-[6-(methylamino)pyridin-2-yl]ethyl}-L-tyrosine

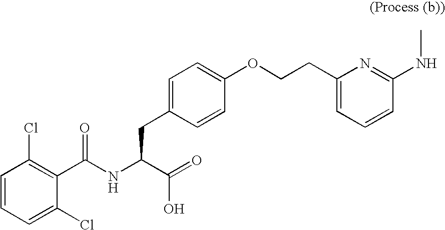

LiOH (126 mg, 3 mmol) and H₂O (0.8 ml) were added to a solution of methyl N-(2,6-dichlorobenzoyl)-O-{2-[6-(methylamino)pyridin-2-yl]ethyl}-L-tyrosinate (500 mg, 1.0 mmol) in DMF (4 ml). After 1.5 hours at room temperature the crude reaction mixture was purified by C18 reverse phase chromatography (basic conditions) to afford the title compound as a white solid (382 mg, 78%); ¹H NMR Data (500 MHz): (DMSO-d6) 2.74 (2H, d), 2.86 (1H, dd), 2.93 (2H, t), 3.05 (1H, dd), 4.25 (2H, t), 4.58 (1H, dd), 6.27 (1H, d), 6.35 (1H, q), 6.43 (1H, d), 6.83 (2H, d), 7.18 (2H, d), 7.30 (1H, dd), 7.38 (1H, dd), 7.42 (1H, s), 7.44 (1H, dd), 8.94 (1H, bs); Mass Spectrum [M+H]⁺=488.

The procedure described above for the preparation of Example 5.1 was repeated using the appropriate methyl ester precursor. Thus were obtained the compounds described below in Table 2:

TABLE 2

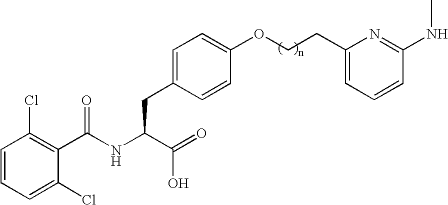

| | | | | Mass | ¹H NMR Data |
|---|---|---|---|---|---|
| Example | n | Name | Yield | Ion | (500 MHz) |
| 5.2 | 3 | N-(2,6- | 58 | 516 | (CDCl3) 1.67-1.82 (4H, |
| | | dichlorobenzoyl)-O- | | [M + H]⁻ | m), 2.56 (2H, t), 2.74 (3H, |
| | | {2-[6- | | | d), 2.88 (1H, dd), 3.06 (1H, |
| | | (methylamino)pyridin- | | | dd), 3.94 (2H, t), 4.55 (1H, |
| | | 2-yl]butyl}-L-tyrosine | | | ddd), 6.23 (1H, d), 7.27- |
| | | | | | 7.33 (1H, m), 6.35 (1H, d), |
| | | | | | 6.80 (2H, d), 7.18 (2H, d), |
| | | | | | 7.28 (1H, dd), 7.38 (1H, |
| | | | | | dd), 7.41-7.45 (2H, m), |
| | | | | | 8.80 (1H, d) |

The starting materials used in Examples 5.1 and 5.2 were prepared as follows:

### Preparation of methyl N-(2,6-dichlorobenzoyl)-O-{2-[6-(methylamino)pyridin-2-yl]ethyl}-L-tyrosinate (Example 5.3)

tert-Butyl [6-(2-hydroxyethyl)pyridin-2-yl]methylcarbamate (515 mg, 2.04 mmol) and triphenylphosphine (780 mg, 3.0 mmol) were added to a solution of methyl N-(2,6-dichlorobenzoyl)-L-tyrosinate (500 mg, 1.36 mmol) in DCM (15 ml). DTAD (656 mg, 2.85 mmol) was added portionwise over 5 minutes, after which the mixture was solubilized. After 60 hours, solvent was evaporated and the residue was purified by silica gel flash chromatography (10 to 40% ethyl acetate in petroleum ether) to afford methyl O-(2-{6-[(tert-butoxycarbonyl)(methyl)amino]pyridin-2-yl}ethyl)-N-(2,6-dichlorobenzoyl)-L-tyrosinate as a white foam (705 mg, 86%); ¹H NMR Spectrum (DMSO-d6) 1.45 (9H, s), 2.87 (1H, dd), 3.04 (1H, dd), 3.12 (2H, t), 3.28 (3H, s), 3.64 (3H, s), 4.31 (2H, t), 4.66 (1H, ddd), 6.84 (2H, d), 7.08 (1H, d), 7.17 (2H, d), 7.40 (1H, dd), 7.43-7.47 (3H, m), 7.68 (1H, dd), 9.17 (1H, d); Mass Spectrum [M−H]⁻=600.

Methyl O-(2-{6-[(tert-butoxycarbonyl)(methyl)amino]pyridin-2-yl}ethyl)-N-(2,6-dichlorobenzoyl)-L-tyrosinate (700 mg, 1.11 mmol) was stirred in TFA (10 ml) for 2 hours at room temperature, then solvent was removed. The residue was dissolved in DCM and NH₃/MeOH 7N was added. Evaporation and dissolution in DCM was followed by a purification by silica gel flash chromatography (1 to 4% NH₃/MeOH 7N in DCM) to afford as Example 5.3 methyl N-(2,6-dichlorobenzoyl)-O-{2-[6-(methylamino) pyridin-2-yl]ethyl}-L-tyrosinate as a white foam (550 mg, 98%); ¹H NMR (500 MHz): (CDCl3) 2.89 (3H, d), 3.05 (2H, t), 3.15-3.25 (2H, m), 3.75 (3H, s), 4.27 (2H, t), 4.54 (1H, bs), 5.14 (1H, ddd), 6.24 (1H, d), 6.26 (1H, d), 6.53 (1H, d), 6.82 (2H, d), 7.08 (2H, d), 7.25 (1H, dd), 7.28-7.33 (2H, m), 7.38 (1H, dd); Mass Spectrum [M+H]⁺=501.

### Preparation of tert-butyl [6-(4-hydroxybutyl)pyridin-2-yl]methylcarbamate

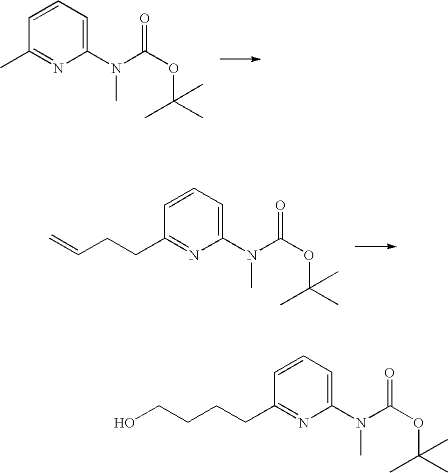

nBuLi 1.6M in hexane (30.9 ml, 49.5 mmol) was added dropwise to a solution of diisopropylamine (7.9 ml, 56.2 mmol) in THF (70 ml) at 0° C. After 0.5 hour at 0° C., this solution was added dropwise over 10 minutes to a solution of tert-butyl methyl(6-methylpyridin-2-yl)carbamate (J. Med. Chem. 2005, 48 (4), 926) (10 g, 45.0 mmol) in THF (100 ml) at −78° C. After 0.5 hour, allylbromide (4.9 ml, 56.2 mmol) was added. The reaction mixture was allowed to stir for another hour at −78° C. and was quenched with an aqueous saturated solution of ammonium chloride. The mixture was extracted with EtOAc, the organic layers combined, dried (MgSO₄) and evaporated. The residue was purified by silica gel flash chromatography (0 to 15% Ethyl acetate in petroleum ether) to afford tert-butyl (6-but-3-en-1-ylpyridin-2-yl)methylcarbamate as a colourless oil (9.3 g, 79%); ¹H NMR Spectrum (DMSO-d6) 1.51 (9H, s), 2.45-2.53 (2H, m), 2.82 (2H, t), 3.39 (3H, s), 4.97 (1H, ddd), 5.05 (1H, ddd), 5.83-5.93 (1H, m), 6.84 (1H, d), 7.42 (1H, d), 7.52 (1H, dd).

A 0.5M solution of 9-BBN in THF (15.2 ml) was added to a solution of tert-butyl (6-but-3-en-1-ylpyridin-2-yl)methylcarbamate (1 g. 3.8 mmol) in THF (10 ml) at room temperature. After 18 hours, the reaction mixture was cooled down to 0° C., a solution of sodium perborate tetrahydrate (1 g, 6.5 mmol) in H₂O (4 ml) was added and the mixture was stirred for 2 hours. After filtration of the salts, the mixture was concentrated, then dissolved in DCM, extracted with an aqueous saturated solution of sodium bicarbonate, dried (MgSO₄) and concentrated. The residue was purified by silica gel flash chromatography (10 to 40% ethyl acetate in petroleum ether) to afford tert-butyl [6-(4-hydroxybutyl)pyridin-2-yl]methylcarbamate as a colourless oil (250 mg, 23%); ¹H NMR Spectrum (CDCl3) 1.51 (9H, s), 1.60-1.68 (2H, m), 1.78-1.86 (2H, m), 2.79 (2H, t), 3.39 (3H, s), 3.69 (2H, t), 6.86 (1H, d), 7.43 (1H, d), 7.54 (1H, dd).

### Preparation of methyl N-(2,6-dichlorobenzoyl)-O-{2-[6-(methylamino)pyridin-2-yl]butyl}-L-tyrosinate (Example 5.4)

tert-Butyl [6-(2-hydroxybutyl)pyridin-2-yl]methylcarbamate (210 mg, 0.75 mmol) and triphenylphosphine (330 mg, 1.25 mmol) were added to a solution of methyl N-(2,6-dichlorobenzoyl)-L-tyrosinate (230 mg, 0.62 mmol) in THF-benzene (2+2 ml) at 0° C. DTAD (288 mg, 1.25 mmol) was added portionwise over 5 minutes, after which the mixture was solubilized. After 1 hour at 0° C., solvent was evaporated and the residue was purified by silica gel flash chromatography (10 to 40% ethyl acetate in petroleum ether) to afford methyl O-(2-{6-[(tert-butoxycarbonyl)(methyl)amino]pyridin-2-yl}butyl)-N-(2,6-dichlorobenzoyl)-L-tyrosinate as a white foam (230 mg, 59%); ¹H NMR Spectrum (CDCl3) 1.51 (9H, s), 1.78-1.94 (4H, m), 2.80 (2H, t), 3.15-3.26 (2H, m), 3.39 (3H, s), 3.75 (3H, s), 3.95 (2H, t), 5.15 (1H, ddd), 6.26 (1H, d), 6.80 (2H, d), 6.86 (1H, d), 7.09 (2H, d), 7.25 (1H, dd), 7.29-7.34 (2H, m), 7.43 (1H, d), 7.53 (1H, dd); Mass Spectrum [M+H]⁺=630.

The procedure described above for the preparation of methyl N-(2,6-dichlorobenzoyl)-O-{2-[6-(methylamino)pyridin-2-yl]ethyl}-L-tyrosinate from methyl O-(2-{6-[(tert-butoxycarbonyl)(methyl)amino]pyridin-2-yl}ethyl)-N-(2,6-dichlorobenzoyl)-L-tyrosinate was repeated by treating methyl O-(2-{6-[(tert-butoxycarbonyl)(methyl)amino]pyridin-2-yl}butyl)-N-(2,6-dichlorobenzoyl)-L-tyrosinate with TFA to give, as Example 5.4, methyl N-(2,6-dichlorobenzoyl)-O-{2-[6-(methylamino)pyridin-2-yl]butyl}-L-tyrosinate (90%); ¹H NMR Spectrum: (CDCl3) 1.78-1.89 (4H, m), 2.66 (2H, t), 2.89 (3H, d), 3.16-3.25 (2H, m), 3.75 (3H, s), 3.93 (2H, t), 4.68 (1H, bs), 5.15 (1H, ddd), 6.22 (1H, d), 6.27 (1H, d), 6.46 (1H, d), 6.79 (2H, d), 7.09 (2H, d), 7.25 (1H, dd), 7.28-7.34 (2H, m), 7.38 (1H, dd); [M+H]⁺=530.

### Example 6

### N-(2,6-dichlorobenzoyl)-O-{3-[6-(methylamino)pyridin-2-yl]propyl}-L-tyrosine

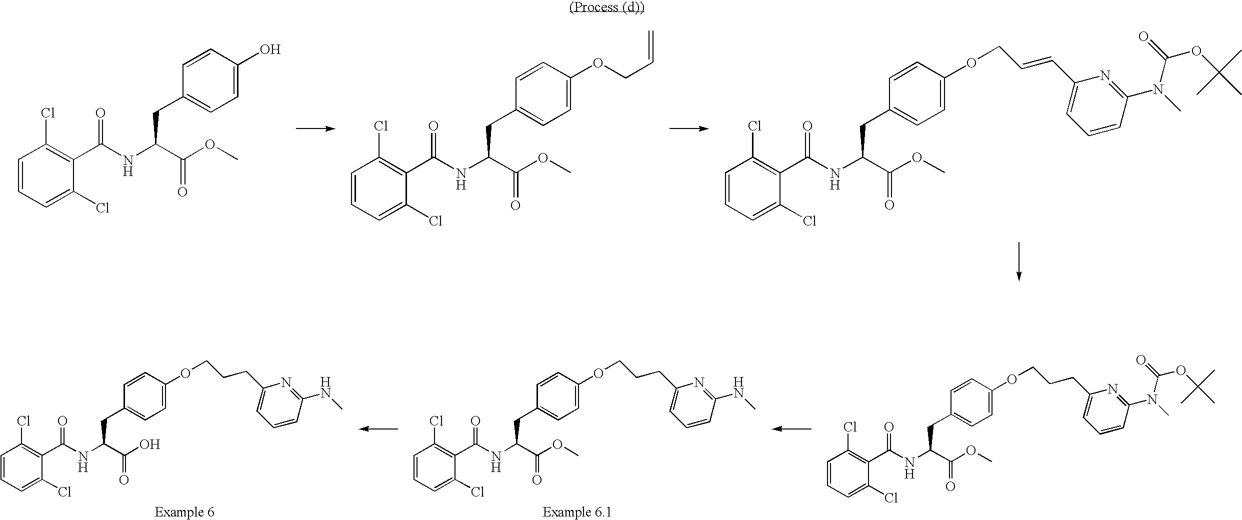

LiOH (50 mg) and H₂O (0.5 ml) were added to a solution of methyl N-(2,6-dichlorobenzoyl)-O-{3-[6-(methylamino)pyridin-2-yl]propyl}-L-tyrosinate (110 mg, 0.21 mmol) in DMA (0.5 ml). After 3 hours at room temperature, the crude reaction mixture was purified by C18 reverse phase chromatography (basic conditions) to afford the title compound as a white solid (40 mg, 37%); ¹H NMR Spectrum: (CDCl3) 1.79-1.97 (2H, m), 2.58 (2H, t), 2.93 (3H, s), 3.26 (1H, dd), 3.40 (1H dd), 3.55-3.67 (2H, m), 4.83 (1H, ddd), 6.40 (1H, d), 6.44 (1H, d), 6.53 (2H, d), 6.87 (1H, d), 7.18 (1H, dd), 7.24-7.29 (4H, m partially hidden by CHCl3), 7.61 (1H, dd), 10.53 (1H, bs); Mass Spectrum [M+H]⁺=502.

The methyl N-(2,6-dichlorobenzoyl)-O-{3-[6-(methylamino)pyridin-2-yl]propyl}-L-tyrosinate used as the starting material was prepared as follows:

Potassium carbonate (564 mg, 4.08 mmol) and allyl bromide (282 μl 3.26 mmol) were added to a solution of methyl N-(2,6-dichlorobenzoyl)-L-tyrosinate (1000 mg, 2.72 mmol) in DMF (25 ml) at room temperature. The reaction mixture was stirred at room temperature for 60 hours, then was filtered and concentrated. The residue was purified by silica gel flash chromatography (0 to 50% Ethyl acetate in petroleum ether) to afford methyl O-allyl-N-(2,6-dichlorobenzoyl)-L-tyrosinate as a white foam (1030 mg, 93%); ¹H NMR Spectrum: (CDCl3) 3.16-3.27 (2H, m), 3.75 (3H, s), 4.48-4.52 (2H, m), 5.15 (1H, ddd), 5.27 (1H, ddd), 5.39 (1H, ddd), 6.03 (1H, ddd), 6.27 (1H, d), 6.83 (2H, d), 7.11 (2H, d), 7.26 (1H, dd partially hidden by CHCl₃), 7.30 (1H, s), 7.31 (1H, dd); Mass Spectrum [M+H]⁺=408.

Potassium carbonate (93 mg, 0.67 mmol), tributylphosphine (30 μl, 0.12 mmol), palladium acetate (14 mg, 0.061 mmol) and tert-butyl [6-bromopyridin-2-yl]methylcarbamate (192 mg, 0.67 mmol) were successively added to a solution of methyl O-allyl-N-(2,6-dichlorobenzoyl)-L-tyrosinate (250 mg, 0.61 mmol) in DMF (6 ml) at room temperature under a nitrogen atmosphere. The reaction mixture was heated at 100° C. for 3 hours. Brine was added to the cooled reaction mixture, and the mixture was extracted with diethyl ether. After evaporation, the residue was purified by silica gel flash chromatography (0 to 30% ethyl acetate in petroleum ether) to afford methyl O-((2E)-3-{6-[(tert-butoxycarbonyl)(methyl)amino]pyridin-2-yl}prop-2-en-1-yl)-N-(2,6-dichlorobenzoyl)-L-tyrosinate as a pale yellow foam (160 mg, 43%); ¹H NMR Spectrum: (CDCl3) 1.55 (9H, s), 3.20-3.24 (2H, m), 3.43 (3H, s), 3.75 (3H, s), 4.71 (2H, dd), 5.16 (1H, ddd), 6.37 (1H, d), 6.72 (1H, ddd), 6.88 (2H, d), 6.92-6.96 (1H, m), 7.13 (2H, d), 7.25-7.33 (4H, m), 7.55 (2H, s); Mass Spectrum [M+H]⁺=614.

A solution of methyl O-((2E)-3-{6-[(tert-butoxycarbonyl)(methyl)amino]pyridin-2-yl}prop-2-en-1-yl)-N-(2,6-dichlorobenzoyl)-L-tyrosinate (160 mg, 0.26 mmol) in methanol (5 ml) was hydrogenated under 1.5 bars hydrogen overnight in the presence of Pt/C (45 mg). The reaction mixture was heated at 100° C. for 3 hours, the catalyst was then filtered off and the resulting mixture evaporated to afford methyl O-(3-{6-[(tert-butoxycarbonyl)(methyl)amino]pyridin-2-yl}propyl)-N-(2,6-dichlorobenzoyl)-L-tyrosinate as a gum (160 mg, 99%); Mass Spectrum [M+H]⁺=616

TFA (1 ml) was added to a solution of methyl O-(3-{6-[(tert-butoxycarbonyl)(methyl)amino]pyridin-2-yl}propyl)-N-(2,6-dichlorobenzoyl)-L-tyrosinate (130 mg, 0.21 mmol) in DCM (1 ml). The reaction mixture was stirred for 2 hours at room temperature, solvent was then removed by evaporation. The residue was dissolved in DCM and 7N NH₃/MeOH was added. White salts were removed by filtration and the solution was evaporated to afford as Example 6.1, methyl N-(2,6-dichlorobenzoyl)-O-{3-[6-(methylamino)pyridin-2-yl]propyl}-L-tyrosinate as a white foam (110 mg, 100%); ¹H NMR Spectrum (DMSO-d6) 2.00-2.10 (2H, m), 2.61-2.68 (2H, m), 2.74 (3H, d), 2.88 (1H, dd), 3.04 (1H, dd), 3.64 (3H, s), 3.96 (2H, t), 4.66 (1H, ddd), 6.23 (1H, d), 6.28-6.33 (1H, m), 6.34 (1H, d), 6.83 (2H, d), 7.17 (2H, d), 7.27 (1H, dd), 7.40 (1H, dd), 7.44 (1H, s), 7.45 (1H, dd), 9.17 (1H, d); Mass Spectrum [M+H]⁺=516.

### Example 7

### N-(2,6-dichlorobenzoyl)-4-[3-(pyridin-2-ylamino)prop-1-yn-1-yl]-L-phenylalanine

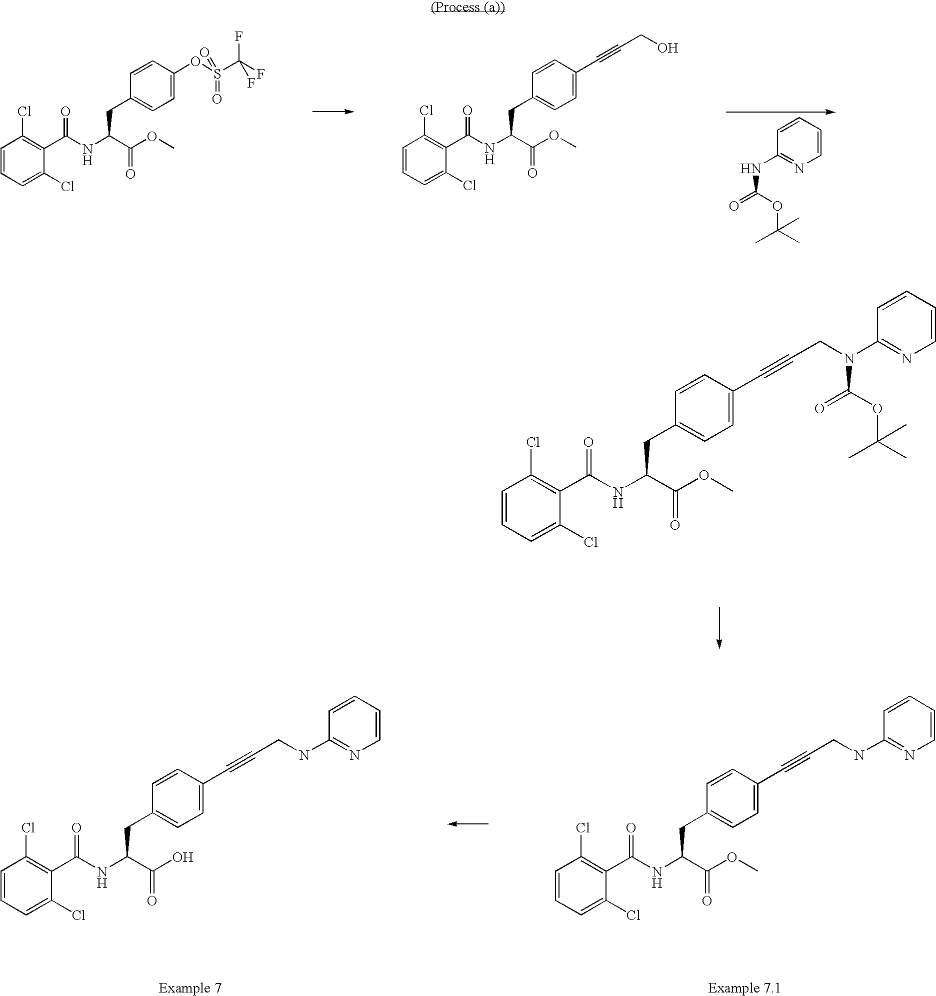

LiOH (8 mg) in solution in H₂O (0.1 ml) was added to a solution of methyl N-(2,6-dichlorobenzoyl)-4-[3-(pyridin-2-ylamino)prop-1-yn-1-yl]-L-phenylalaninate (90 mg, 0.19 mmol) in methanol (0.3 ml) and DMA (0.3 ml). After stirring at room temperature for 2 hours, the reaction mixture was purified by C18 reverse phase chromatography (basic conditions) to give the title compound as a white solid (45 mg, 52%); ¹H NMR Spectrum: (DMSO-d6) 2.92 (1H, dd), 3.14 (1H, dd), 4.29 (2H, d), 4.63 (1H, ddd), 6.53-6.58 (2H, m), 6.91 (1H, t), 7.25-7.32 (4H, m), 7.36-7.45 (4H, m), 8.03 (1H, dd), 8.94 (1H, d); Mass Spectrum [M−H]⁻=466.

The methyl N-(2,6-dichlorobenzoyl)-4-[3-(pyridin-2-ylamino)prop-1-yn-1-yl]-L-phenylalaninate used as starting material was prepared as follows:

Propargylic alcohol (0.23 ml, 4 mmol) was added to a solution of methyl N-(2,6-dichlorobenzoyl)-O-[(trifluoromethyl)sulfonyl]-L-tyrosinate (1 g. 2 mmol), CuI (15 mg, 0.08 mmol), triethylamine (1.12 ml, 8 mmol) and Pd(Cl₂)(PPh₃)₂ (84 mg, 0.12 mmol) in DMF (10 ml) under an argon atmosphere. The mixture was heated at 80° C. for 2 hours. After evaporation, the residue was extracted with ethyl acetate and purified by silica gel flash chromatography eluting with 0 to 30% ethyl acetate in DCM to give methyl N-(2,6-dichlorobenzoyl)-4-(3-hydroxyprop-1-yn-1-yl)-L-phenylalaninate as a foam (343 mg, 42%); ¹H NMR Spectrum: (DMSO-d6) 2.95 (1H, dd), 3.16 (1H, dd), 3.66 (3H, s), 4.29 (2H, d), 4.76 (1H, ddd), 5.31 (1H, t), 7.29 (2H, d), 7.34 (2H, d), 7.40 (1H, dd), 7.43-7.47 (2H, m), 9.20 (1H, d); Mass Spectrum [M−H]⁻=404.

Triethylamine (82 μl, 0.59 mmol) and mesyl chloride (36 μl, 0.47 mmol) were added to a solution of methyl N-(2,6-dichlorobenzoyl)-4-(3-hydroxyprop-1-yn-1-yl)-L-phenylalaninate (160 mg, 0.39 mmol) in DCM (1 ml), under an argon atmosphere and at 0° C. After 2 hours, the mixture was extracted with methylene chloride, evaporated and dried on MgSO₄. The residue was dissolved in DMA and added to a solution of tert-butyl pyridin-2-ylcarbamate anion prepared by treating, under argon atmosphere at 0° C., tert-butyl pyridin-2-ylcarbamate (153 mg, 0.79 mmol) in solution in DMA (2 ml) with NaH 60% in oil (30 mg, 0.74 mmol). The reaction mixture was stirred at room temperature for 3 hours, extracted with ethyl acetate and purified by silica gel flash chromatography eluting with 0 to 50% ethyl acetate in petroleum ether to give methyl 4-{3-[(tert-butoxycarbonyl)(pyridin-2-yl)amino]prop-1-yn-1-yl}-N-(2,6-dichlorobenzoyl)-L-phenylalaninate as an oil (112 mg, 49%); ¹H NMR Spectrum: (DMSO-d6) 1.50 (9H, s), 2.93 (1H, dd), 3.14 (1H, dd), 3.65 (3H, s), 3.74 (1H, ddd), 4.88 (2H, s), 7.18 (1H, dd), 7.22-7.30 (4H, m), 7.39 (1H, dd), 7.42-7.46 (2H, m), 7.67 (1H, d), 7.80 (1H, ddd), 8.43 (1H, ddd), 9.18 (1H, d); Mass Spectrum [M+H]⁺=582.

Methyl 4-{3-[(tert-butoxycarbonyl)(pyridin-2-yl)amino]prop-1-yn-1-yl}-N-(2,6-dichlorobenzoyl)-L-phenylalaninate (112 mg, 0.19 mmol) in solution in DCM (1 ml) was treated with TFA (1 ml). The solution was stirred overnight. The solvent was evaporated and ethyl acetate was added. After neutralization to pH 7 with NaHCO₃, extraction and evaporation, to give, as Example 7.1, methyl N-(2,6-dichlorobenzoyl)-4-[3-(pyridin-2-ylamino)prop-1-yn-1-yl]-L-phenylalaninate as an oil (93 mg, 97%) which was used in the next step without further purification; ¹H NMR Spectrum: (DMSO-d6) 2.94 (1H, dd), 3.14 (1H, dd), 3.65 (3H, s), 4.29 (2H, d), 4.74 (1H, ddd), 6.52-6.58 (2H, m), 6.91 (1H, t), 7.26 (2H, d), 7.30 (2H, d), 7.38-7.43 (2H, m), 7.43-7.47 (2H, m), 8.03 (1H, dd), 9.19 (1H, d); Mass Spectrum [M+H]⁺=482.

### Example 8

### N-(2,6-dichlorobenzoyl)-4-[3-(Pyridin-2-ylamino)propyl]-L-phenylalanine

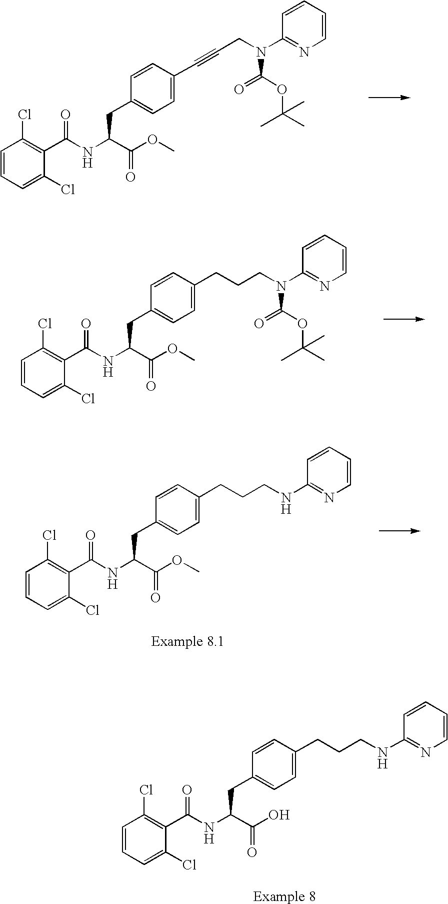

LiOH (40 mg) in solution in H₂O (0.1 ml) was added to a solution of methyl N-(2,6-dichlorobenzoyl)-4-[3-(pyridin-2-ylamino)propyl]-L-phenylalaninate (47 mg, 0.10 mmol) in methanol (0.5 ml) and DMA (0.6 ml) was added. After stirring at room temperature for 5 hours, the reaction mixture was purified by C18 reverse phase chromatography (basic conditions) to give the title product as a white solid (27 mg, 56%); ¹H NMR Spectrum: (DMSO-d6) 1.72-1.83 (2H, m), 2.58 (2H, t), 2.95 (1H, dd), 3.10 (1H, dd), 3.17-3.24 (2H, m), 4.40 (1H, bs), 6.40-6.45 (2H, m), 6.48 (1H, m), 7.06 (2H, d), 7.17 (2H, d), 7.33 (1H, ddd), 7.36 (1H, dd), 7.39-7.43 (2H, m), 7.94 (1H, dd), 8.33 (1H, bs); Mass Spectrum [M+H]⁺=472.

The methyl N-(2,6-dichlorobenzoyl)-4-[3-(pyridin-2-ylamino)propyl]-L-phenylalaninate used as the starting material was prepared as follows:

10% Pt/C (25 mg) was added to a solution of methyl 4-{3-[(tert-butoxycarbonyl)(pyridin-2-yl)amino]prop-1-yn-1-yl}-N-(2,6-dichlorobenzoyl)-L-phenylalaninate (80 mg, 0.14 mmol) in methanol (3 ml). The reaction mixture was stirred under hydrogen at 1.5 atmosphere for 5 hours. The solution was filtered through a pad of Celite and the filtrate was concentrated to afford methyl 4-{3-[(tert-butoxycarbonyl)(pyridin-2-yl)amino]propyl}-N-(2,6-dichlorobenzoyl)-L-phenylalaninate as an oil (79 mg, 98%); ¹H NMR Spectrum: (DMSO-d6) 1.43 (9H, s), 1.76-1.86 (2H, m), 2.53 (2H, t), [partially hidden by DMSO] 2.89 (1H, dd), 3.08 (1H, dd), 3.64 (3H, s), 3.83 (2H, t), 4.69 (1H, ddd), 7.05 (2H, d), 7.14 (1H, dd), 7.16 (2H, d), 7.39 (1H, dd), 7.41-7.45 (2H, m), 7.53 (1H, d), 7.75 (1H, ddd), 8.38 (1H, dd), 9.17 (1H, d); Mass Spectrum [M−H]⁻=584.

Methyl 4-{3-[(tert-butoxycarbonyl)(pyridin-2-yl)amino]propyl}-N-(2,6-dichlorobenzoyl)-L-phenylalaninate (112 mg, 0.19 mmol) in solution in DCM (0.3 ml) was treated with TFA (1 ml). The solution was stirred at room temperature for 5 hours. The solvent was evaporated and ethyl acetate was added. After neutralization to pH 7 with NaHCO₃, extraction and evaporation, methyl N-(2,6-dichlorobenzoyl)-4-[3-(pyridin-2-ylamino)propyl]-L-phenylalaninate (Example 8.1) was obtained as a solid (50 mg, 79%) and used in the next step without further purification; ¹H NMR Spectrum: (DMSO-d6) 1.73-1.84 (2H, m), 2.60 (2H, t), 2.91 (1H, dd), 3.09 (1H, dd), 3.16-3.24 (2H, m), 3.65 (3H, s), 4.71 (1H, ddd), 6.40-6.46 (2H, m), 7.12 (2H, d), 7.19 (2H, d), 7.33 (1H, ddd), 7.39 (1H, dd), 7.41-7.46 (2H, m), 7.94 (1H, dd), 9.18 (1H, d): Mass Spectrum [M+H]⁺=486.

### Example 9

### N-(2,6-dichlorobenzoyl)-4-[5-(Pyridin-2-ylamino)pent-1-yn-1-yl]-L-phenylalanine

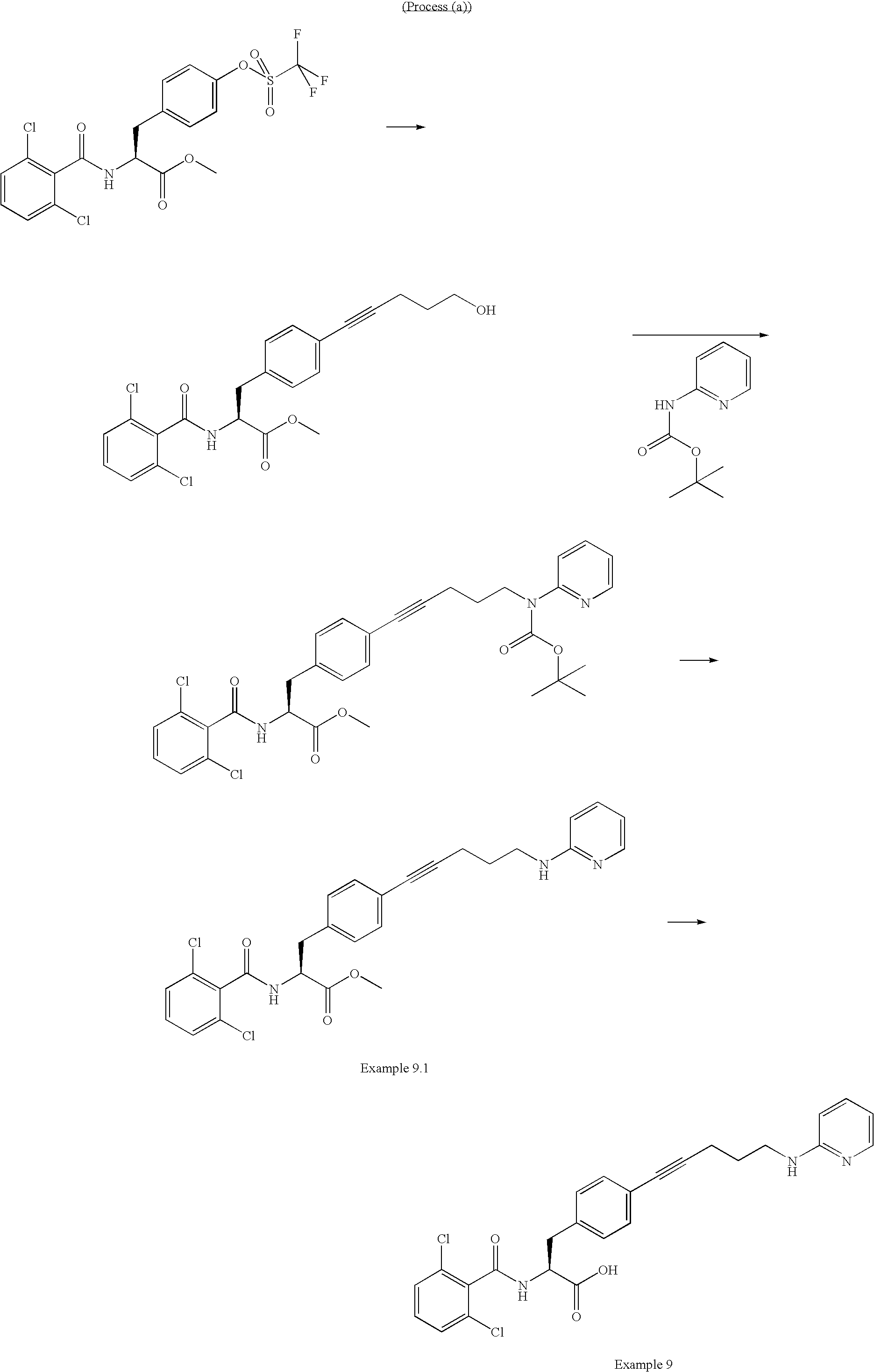

LiOH (60 mg) in solution in H₂O (0.1 ml) was added to a solution of methyl N-(2,6-dichlorobenzoyl)-4-[5-(pyridin-2-ylamino)pent-1-yn-1-yl]-L-phenylalaninate (65 mg, 0.13 mmol) in methanol (0.5 ml) and DMA (0.8 ml). After stirring at room temperature for 5 hours, the reaction mixture was purified by C18 reverse phase chromatography (basic conditions) to give the title product as a white solid (40 mg, 63%); ¹H NMR Spectrum: (DMSO-d6) 1.73-1.83 (2H, m), 2.48 (2H, t), [partially hidden by DMSOd5] 2.98 (1H, dd), 3.13 (1H, dd), 3.29-3.37 (2H, m), 4.39-4.48 (1H, m), 6.41-6.48 (2H, m), 6.55 (1H, m), 7.20-7.28 (4H, m), 7.34 (1H, ddd), 7.37 (1H, dd), 7.40-7.45 (2H, m), 7.95 (1H, ddd), 8.40 (1H, bs); Mass Spectrum [M+H]⁺=496.

The methyl N-(2,6-dichlorobenzoyl)-4-[5-(pyridin-2-ylamino)pent-1-yn-1-yl]-L-phenylalaninate used as the starting material was prepared as follows:

Pent-4-yn-1-ol (744 μl, 8 mmol) was added to a solution of methyl N-(2,6-dichlorobenzoyl)-O-[(trifluoromethyl)sulfonyl]-L-tyrosinate (2 g, 4 mmol), CuI (30 mg, 0.16 mmol), triethylamine (2.23 ml, 16 mmol) and Pd(Cl₂(PPh₃)₂ (168 mg, 0.24 mmol) in DMF (20 ml), under an argon atmosphere. The mixture was heated at 80° C. overnight. After evaporation, the residue was extracted with ethyl acetate and purified by silica gel flash chromatography eluting with 0 to 30% ethyl acetate in DCM to give methyl N-(2,6-dichlorobenzoyl)-4-(5-hydroxypent-1-yn-1-yl)-L-phenylalaninate as a foam (1.12 g, 67%); ¹H NMR Spectrum: (DMSO-d6) 1.62-1.72 (2H, m), 2.44 (2H, t), 2.94 (1H, dd), 3.14 (1H, dd), 3.46-3.55 (2H, m), 3.65 (3H, s), 4.53 (1H, t), 4.74 (1H, ddd), 7.25 (2H, d), 7.29 (2H, d), 7.40 (1H, dd), 7.43-7.48 (2H, m), 9.20 (1H, d); Mass Spectrum [M+H]⁺=434.

Triethylamine (258 μl, 1.85 mmol) and mesyl chloride (115 μl, 1.48 mmol) were added to a solution of methyl N-(2,6-dichlorobenzoyl)-4-(5-hydroxypent-1-yn-1-yl)-L-phenylalaninate (500 mg, 1.23 mmol) in methylene chloride (5 ml) under argon atmosphere at 0° C. After 2 hours, the mixture was extracted with DCM, evaporated and dried on MgSO₄. The residue was re-dissolved in DMA and added to a solution of tert-butyl pyridin-2-ylcarbamate anion prepared by treating under argon atmosphere at 0° C. tert-butyl pyridin-2-ylcarbamate (477 mg, 2.46 mmol) in solution in DMA (7 ml) with NaH 60% (98 mg, 2.46 mmol). The reaction mixture was stirred at 40° C. for 11 hours, extracted with ethyl acetate and purified by silica gel flash chromatography eluting with 0 to 50% ethyl acetate in petroleum ether to give methyl 4-{5-[(tert-butoxycarbonyl)(pyridin-2-yl)amino]pent-1-yn-1-yl}-N-(2,6-dichlorobenzoyl)-L-phenylalaninate as an oil (208 mg, 28%); ¹H NMR Spectrum: (DMSO-d6) 1.45 (9H, s), 1.77-1.87 (2H, m), 2.43 (2H, t), 2.94 (1H, dd), 3.13 (1H, dd), 3.65 (3H, s), 3.98 (2H, d), 4.74 (1H, ddd), 7.14 (1H, dd), 7.22-7.29 (4H, m), 7.40 (1H, dd), 7.42-7.47 (2H, m), 7.58 (1H, d), 7.76 (1H, ddd), 8.38 (1H, ddd), 9.20 (1H, d); Mass Spectrum: [M+H]⁺=610.

Methyl 4-{5-[(tert-butoxycarbonyl)(pyridin-2-yl)amino]pent-1-yn-1-yl}-N-(2,6-dichlorobenzoyl)-L-phenylalaninate (187 mg, 0.31 mmol) in solution in DCM (0.5 ml) was treated with TFA (1.5 ml). The solution was stirred at room temperature for 3 hours. The solvent was evaporated and ethyl acetate was added. After neutralization to pH 7 with NaHCO₃, extraction and evaporation, methyl N-(2,6-dichlorobenzoyl)-4-[5-(pyridin-2-ylamino)pent-1-yn-1-yl]-L-phenylalaninate (Example 8.1) was obtained as a solid (113 mg, 72%) and used in the next step without further purification; ¹H NMR Spectrum: (DMSO-d6) 1.74-1.84 (2H, m), 2.48 (2H, t), [partially hidden by DMSOd5] 2.94 (1H, dd), 3.14 (1H, dd), 3.30-3.37 (2H, m), [partially hidden by H₂O] 3.65 (3H, s), 4.74 (1H, ddd), 6.42-6.48 (2H, m), 6.54 (1H, t), 7.25 (2H, d), 7.30 (2H, d), 7.34 (1H, ddd), 7.40 (1H, dd), 7.43-7.44 (2H, m), 7.95 (1H, dd), 9.20 (1H, d); Mass Spectrum [M+H]⁺=510.

### Example 10

### O-[2-(1H-benzimidazol-2-ylamino)ethyl]-N-(2,6-dichlorobenzoyl)-L-tyrosine

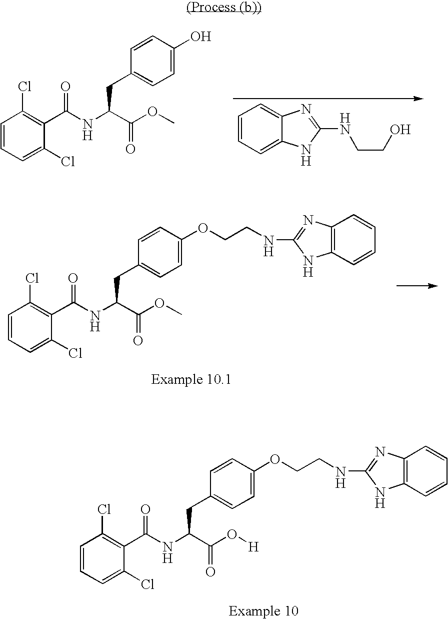

LiOH (0.04 g, 1.06 mmol) was added to a stirred solution of methyl O-[2-(1H-benzimidazol-2-ylamino)ethyl]-N-(2,6-dichlorobenzoyl)-L-tyrosinate (0.13 g, 0.35 mmol) in MeOH/water (3 ml, 2:1). The reaction mixture was stirred for 1 hour at room temperature, concentrated under reduced pressure, then dissolved in DMF (2 ml) and acidified with formic acid before purification by C18 reverse phase chromatography (acidic conditions) to afford the title compound (35 mg, 19%); ¹H NMR Spectrum: (DMSO-d6) 2.92 (1H, dd), 3.06 (1H, dd), 3.61-3.67 (2H, m), 4.09 (2H, t), 4.43 (1H, dd), 6.81-6.89 (5H, m), 7.10-7.15 (2H, m), 7.18 (2H, d), 7.37 (1H, dd), 7.40 (1H, s), 7.41 (1H, dd), 8.48 (1H, bs); Mass Spectrum: [M+H]⁺=513.

The methyl O-[2-(1H-benzimidazol-2-ylamino)ethyl]-N-(2,6-dichlorobenzoyl)-L-tyrosinate used as starting material was prepared as follows:

Triphenylphosphine (0.27 g, 1 mmol) and 2-(2-benzimidazoylamino)-1-ethanol (0.073 g, 0.41 mmol) were added to a solution of methyl N-(2,6-dichlorobenzoyl)-L-tyrosinate (0.1 g, 0.27 mmol) in DCM (5 ml) at 0° C. Then, DTAD was added (0.21 g, 0.94 mmol). The reaction mixture was allowed to stir at 50° C. for 2 hours, then was concentrated and purified by C18 reverse phase chromatography (acidic conditions) to afford as Example 10.1 methyl O-[2-(1H-benzimidazol-2-ylamino)ethyl]-N-(2,6-dichlorobenzoyl)-L-tyrosinate which was used in the next step; Mass Spectrum [M+H]⁺=527.

### Example 11

### N-(2,6-dichlorobenzoyl)-4-[(5,6,7,8-tetrahydro-1,8-naphthyridin-2-ylacetyl)amino]-L-phenylalanine

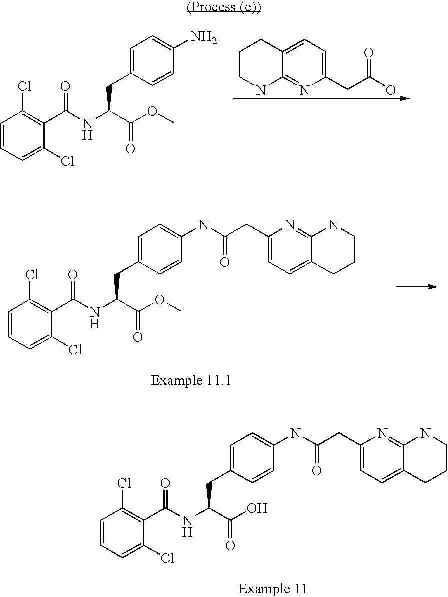

LiOH (17 mg, 0.41 mmol) was added to a solution of methyl N-(2,6-dichlorobenzoyl)-4-[(5,6,7,8-tetrahydro-1,8-naphthyridin-2-ylacetyl)amino]-L-phenylalaninate (110 mg, 0.2 mmol) in DMA (1 ml). After stirring at room temperature overnight, the reaction mixture was purified by C18 reverse phase chromatography (basic conditions) to give the title compound as a white solid (53 mg, 50%); ¹H NMR Spectrum: (DMSO-d6) 1.71-1.81 (2H, m), 2.62 (2H, t), 2.88 (1H, dd), 3.07 (1H, dd), 3.25 (2H, bs), 3.50 (2H, s), 4.61 (1H, ddd), 6.38 (1H, d), 6.45 (1H, bs), 7.08 (1H, d), 7.20 (2H, d), 7.39 (1H, dd), 7.43 (2H, s), 7.44 (1H, dd), 7.51 (2H, dd), 8.99 (1H, d); Mass Spectrum: [M+H]⁺=527.

The methyl N-(2,6-dichlorobenzoyl)-4-[(5,6,7,8-tetrahydro-1,8-naphthyridin-2-ylacetyl)amino]-L-phenylalaninate used as the starting material was prepared as follows:

Methyl 4-amino-N-(2,6-dichlorobenzoyl)-L-phenylalaninate (200 mg, 0.54 mmol), HOPO (60 mg, 0.54 mmol) and EDCI done (115 mg, 0.60 mmol) were added to a suspension of 5,6,7,8-tetrahydro-1,8-naphthyridin-2-ylacetic acid (112 mg, 0.49 mmol) in methylene chloride (2 ml) was added at 0° C. under argon atmosphere. The reaction mixture was stirred at 0° C. for 1 hour and at room temperature for 2 hours. The mixture was partitioned between KHSO₄ (1M) and methylene chloride. The organic phase was washed with brine, dried and evaporated to dryness. The residue was purified by silica gel flash chromatography eluting with 25% ethyl acetate in DCM to give as Example 11.1, methyl N-(2,6-dichlorobenzoyl)-4-[(5,6,7,8-tetrahydro-1,8-naphthyridin-2-ylacetyl)amino]-L-phenylalaninate (121 mg, 41%); ¹H NMR Spectrum: (DMSO-d6) 1.70-1.81 (2H, m), 2.62 (2H, t), 2.90 (1H, dd), 3.06 (1H, dd), 3.21-3.28 (2H, m), 3.51 (2H, s), 3.64 (3H, s), 4.68 (1H, ddd), 6.38 (1H, d), 6.43 (1H, bs), 7.08 (1H, d), 7.19 (2H, d), 7.41 (1H, dd), 7.45 (1H, s), 7.46 (1H, dd), 7.52 (2H, d), 9.20 (1H, d), 10.14 (1H, s); Mass Spectrum [M+H]⁺=541.

### Example 12

### N-(2-chlorobenzoyl)-O-[3-(pyridin-2-ylamino)propyl]-L-tyrosine

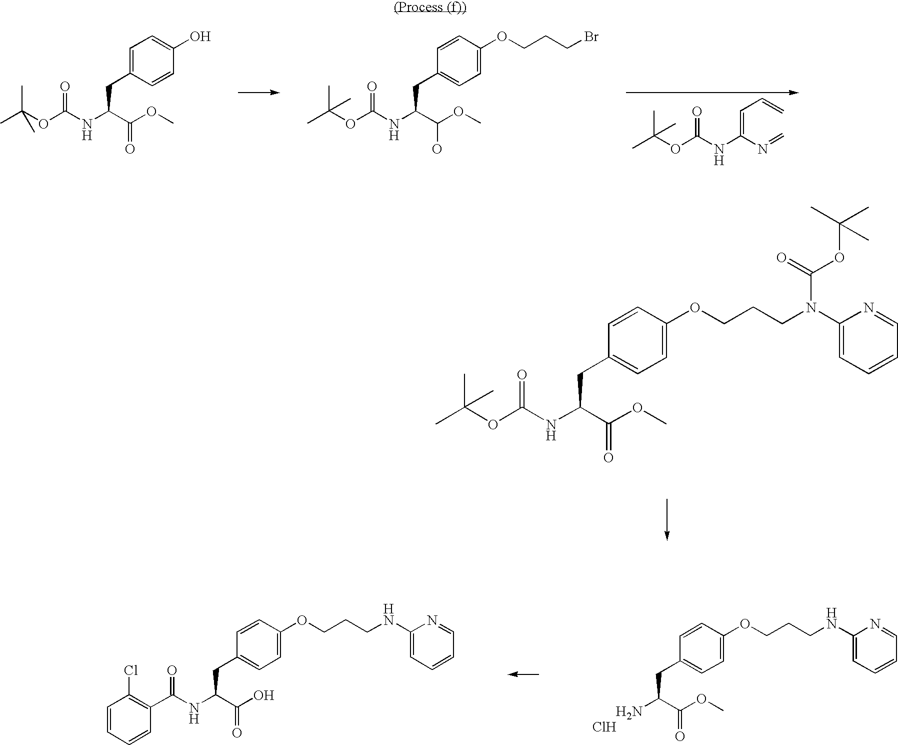

TBTU (141 mg, 0.37 mmol) was added to a solution of methyl O-[3-(pyridin-2-ylamino)propyl]-L-tyrosinate hydrochloride (100 mg, 0.25 mmol), 2-chlorobenzoic acid (58 mg, 0.37 mmol) and N-methylmorpholine (120 μl, 1.09 mmol) in DMF (1 ml). The reaction mixture was stirred at room temperature for 72 hours, then LiOH (31 mg, 0.74 mmol) and water (0.2 ml) were added, and the mixture was stirred for 24 hours. The reaction mixture was directly purified by C18 reverse phase chromatography (basic conditions) to give the title compound as a white solid (53 mg, 47%); ¹H NMR Spectrum: (DMSO-d6) 1.89-2.00 (2H, m), 2.95 (1H, dd), 3.12 (1H dd), 3.36 (2H, dd), 3.99 (2H, t), 4.20 (1H, bs), 6.41-6.47 (2H, m), 6.54 (1H, t), 6.79 (2H, d), 7.12 (2H, d), 7.28-7.46 (5H, m), 7.85 (1H, bs), 7.95 (1H, dd); Mass Spectrum: [M+H]⁺=454.

The procedure described above was repeated using the appropriate commercially available acids. Thus were obtained the compounds described below in Table 3:

TABLE 3

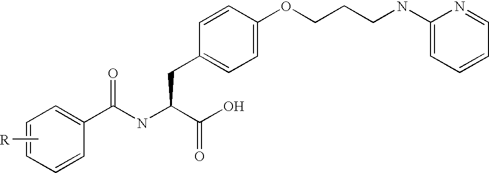

| | | | | Mass | ¹H NMR Data |
|---|---|---|---|---|---|
| Example | R | Name | Yield | Ion | (500 MHz) |
| 12.1 | 2,4- | N-(2,4- | 36 | 486 | (DMSO-d6) 1.80-1.89 |
| | dichloro | dichlorobenzoyl)- | | [M − H]− | (2H, m), 2.76 (1H, |
| | | O-[3(pyridin-2- | | | dd), 2.98 (1H, dd), |
| | | ylamino)propyl]- | | | 3.26 (2H, bs partially |
| | | L-tyrosine | | | hidden by H2O), 3.90 |
| | | | | | (2H, t), 4.40 (1H, |
| | | | | | ddd), 6.33 (1H, bs), |
| | | | | | 6.34 (1H, s), 6.43 (1H, |
| | | | | | t), 6.74 (2H, d), 7.07 |
| | | | | | (2H, d) 7.14 (1H, d), |
| | | | | | 7.22 (1H, ddd), 7.37 |
| | | | | | (1H, dd), 7.53 (1H, d), |
| | | | | | 7.84 (1H, dd), 8.64 |
| | | | | | (1H, d) |
| 12.2 | 2-chloro- | N-(2-chloro-5- | 37 | 468 | (DMSO-d6) 1.92-2.01 |
| | 5-methyl | methylbenzoyl)- | | [M + H]⁺ | (2H, m), 2.29 (3H, s), |
| | | O-[3-(pyridin-2- | | | 2.90 (1H, dd), 3.10 |
| | | ylamino)propyl]- | | | (1H, dd), 3.34-3.40 |
| | | L-tyrosine | | | (2H, m), 4.01 (2H, t), |
| | | | | | 4.45 (1H, ddd), 6.44 |
| | | | | | (1H, ddd), 6.46 (1H, |
| | | | | | s), 6.55 (1H, t), 6.85 |
| | | | | | (2H, d), 7.04 (1H, d), |
| | | | | | 7.18 (2H, d), 7.26 |
| | | | | | (1H, dd), 7.30-7.30 |
| | | | | | (2H, m), 7.95 (1H, |
| | | | | | dd), 8.45 (1H, bs) |
| 12.3 | 2-chloro- | N-(2-chloro-6- | 38 | 472 | (DMSO-d6) 1.92-2.01 |
| | 6-fluoro | fluorobenzoyl)-O- | | [M + H]⁺ | (2H, m), 2.88 (1H, |
| | | [3-(pyridin-2- | | | dd), 3.06 (1H, dd), |
| | | ylamino)propyl]- | | | 3.34-3.39 (2H, m), |
| | | L-tyrosine | | | 4.01 (2H, t), 4.50 (1H, |
| | | | | | ddd), 6.45 (1H, dd), |
| | | | | | 6.46 (1H, s), 6.55 (1H, |
| | | | | | t), 6.83 (2H, d), 7.18 |
| | | | | | (2H, d), 7.23-7.28 |
| | | | | | (1H, m), 7.32 (1H, d), |
| | | | | | 7.34 (1H, ddd), 7.45 |
| | | | | | (1H, ddd), 7.96 (1H, |
| | | | | | dd), 8.86 (1H, bs) |
| 12.4 | 2-chloro- | N-(2-chloro-6- | 44 | 486 | (DMSO-d6) 1.91-1.99 |
| | 6-fluoro- | fluoro-3- | | [M + H]⁺ | (2H, m), 2.29 (3H, s), |
| | 3-methyl- | methylbenzoyl)- | | | 2.94 (1H, dd), 3.08 |
| | | O-[3-(pyridin-2- | | | (1H, dd), 3.34-3.41 |
| | | ylamino)propyl]- | | | (2H, m), 4.00 (2H, t), |
| | | L-tyrosine | | | 4.29 (1H, dd), 6.42- |
| | | | | | 6.47 (2H, m), 6.55 |
| | | | | | (1H, t), 6.78 (2H, d), |
| | | | | | 7.11-7.18 (3H, m), |
| | | | | | 7.34 (1H, ddd), 7.39 |
| | | | | | (1H, dd), 7.96 (1H, |
| | | | | | dd), 8.11 (1H, bs) |
| 12.5 | 6-chloro | N-(6-chloro-2- | 41 | 486 | (DMSO-d6) 1.92-2.00 |
| | 2-fluoro- | fluoro-3- | | [M + H]⁺ | (2H, m), 2.22 (3H, s), |
| | 3-methyl- | methylbenzoyl)- | | | 2.88 (1H, dd), 3.06 |
| | | O-[3-(pyridin-2- | | | (1H, dd), 3.35-3.40 |
| | | ylamino)propyl]- | | | (2H, m), 4.01 (2H, t), |
| | | L-tyrosine | | | 4.53 (1H, ddd), 6.45 |
| | | | | | (1H, dd), 6.46 (1H, s), |
| | | | | | 6.55 (1H, t), 6.84 (2H, |
| | | | | | d), 7.16-7.22 (3H, m), |
| | | | | | 7.30-7.37 (2H, m), |
| | | | | | 7.96 (1H, dd), 8.90 |
| | | | | | (1H, bs) |
| 12.6 | 3-chloro- | N-(3-chloro-2- | 40 | 468 | (DMSO-d6) 1.92-2.01 |
| | 2-methyl | methylbenzoyl)- | | [M + H]⁺ | (2H, m), 2.19 (3H, s), |
| | | O-[3-(pyridin-2- | | | 2.90 (1H, dd), 3.14 |
| | | ylamino)propyl]- | | | (1H, dd), 3.35-3.40 |
| | | L-tyrosine | | | (2H, m), 4.02 (2H, t), |
| | | | | | 4.31 (1H, ddd), 6.43- |
| | | | | | 6.48 (2H, m), 6.56 |
| | | | | | (1H, t), 6.84 (2H, d), |
| | | | | | 7.09 (1H, dd), 7.15 |
| | | | | | (2H, d), 7.24 (1H, dd), |
| | | | | | 7.35 (1H, ddd), 7.46 |
| | | | | | (1H, dd), 7.97 (1H, |
| | | | | | dd), 8.01 (1H, bs) |
| 12.7 | 2-chloro- | N-(2-chloro-3,6- | 39 | 490 | (DMSO-d6) 1.92- |
| | 3,6- | difluorobenzoyl)- | | [M + H]⁺ | 2.01 (2H, m), 2.83 |
| | difluoro | O-[3-(pyridin-2- | | | (1H, dd), 3.08 (1H, |
| | | ylamino)propyl]- | | | dd), 3.34-3.39 (2H, |
| | | L-tyrosine | | | m), 4.01 (2H, t), 4.56 |
| | | | | | (1H, ddd), 6.43-6.47 |
| | | | | | (2H, m), 6.55 (1H, t), |
| | | | | | 6.85 (2H, d), 7.18 |
| | | | | | (2H, d), 6.31-7.38 |
| | | | | | (2H, m), 7.52 (1H, |
| | | | | | ddd), 7.96 (1H, dd), |
| | | | | | 9.07 (1H, d) |
| 12.8 | 2-methyl | N-(2- | 38 | 434 | (DMSO-d6) 1.91-1.99 |
| | | methylbenzoyl)- | | [M + H]⁺ | (2H, m), 2.19 (3H, s), |
| | | O-[3-(pyridin-2- | | | 2.92 (1H, dd), 3.12 |
| | | ylamino)propyl]- | | | (1H, dd), 3.34-3.41 |
| | | L-tyrosine | | | (2H, m), 4.01 (2H, t), |
| | | | | | 4.38 (1H, ddd), 6.44 |
| | | | | | (1H, ddd), 6.46 (1H, |
| | | | | | ddd), 6.55 (1H, t), |
| | | | | | 6.83 (2H, d), 7.14- |
| | | | | | 7.21 (m, 5H), 7.29 |
| | | | | | (1H, ddd), 7.34 (1H, |
| | | | | | ddd), 7.95 (1H, dd), |
| | | | | | 8.00 (1H, bs) |
| 12.9 | 2,4,6- | N- | 35 | 462 | (DMSO-d6) 1.90- |
| | trimethyl | (mesitylcarbonyl)- | | [M + H]⁺ | 2.00 (8H, m), 2.19 |
| | | O-[3-(pyridin-2- | | | (3H, s), 2.83 (1H, dd), |
| | | ylamino)propyl]- | | | 3.09 (1H, dd), 3.32- |
| | | L-tyrosine | | | 3.38 (2H, m), 4.00 |
| | | | | | (2H, t), 4.45 (1H, |
| | | | | | ddd), 6.42-6.47 (2H, |
| | | | | | m), 6.54 (1H, t), 6.75 |
| | | | | | (2H, s), 6.81 (d, 2H), |
| | | | | | 7.16 (2H, d), 7.34 |
| | | | | | (1H, ddd), 7.95 (1H, |
| | | | | | dd), 8.00 (1H, bs) |
| 12.10 | 2,6- | N-(2,6-dichloro-3- | 42% | 502 | (DMSO-d6) 1.98-2.03 |
| | dichloro- | methylbenzoyl)-O- | | [M + H]⁺ | (m, 2H), 2.29 (s, 3H), |
| | 3-methyl | [3-(pyridin-2- | | | 2.88 (dd, 1H), 3.04 |
| | | ylamino)propyl]- | | | (dd, 1H), 3.35-3.41 (m |
| | | L-tyrosine | | | partially hidden by |
| | | | | | H2O, 2H), 4.01 (t, |
| | | | | | 2H), 4.54 (bs, 1H), |
| | | | | | 6.41-6.48 (m, 2H), |
| | | | | | 6.82 (d, 2H), 7.18 (d, |
| | | | | | 2H), 7.30-7.40 (m, |
| | | | | | 3H), 7.95 (d, 1H), |
| | | | | | 8.80 (bs, 1H), 12.65 |
| | | | | | (bs, 1H) |
| 12.11 | 2,6- | N-(2,6- | 38% | 448 | (DMSO-d6) 1.92-1.99 |
| | dimethyl | dimethylbenzoyl)- | | [M + H]⁺ | (m, 2H), 2.01 (s, 6H), |
| | | O-[3-(pyridin-2- | | | 2.84 (dd, 1H), 3.11 |
| | | ylamino)propyl]- | | | (dd, 1H), 3.33-3.40 (m |
| | | L-tyrosine | | | partially hidden by |
| | | | | | H2O, 2H), 4.01 (t, |
| | | | | | 2H), 4.42-4.50 (m, |
| | | | | | 1H), 6.43-6.48 (m, |
| | | | | | 2H), 6.54 (t, 1H), 6.82 |
| | | | | | (d, 2H), 6.94 (d, 2H), |
| | | | | | 7.10 (t, 1H), 7.17 (d, |
| | | | | | 2H), 7.34 (ddd, 1H), |
| | | | | | 7.96 (dd, 1H), 8.04 |
| | | | | | (bs, 1H) |
| 12.12 | 2,3- | N-[(5-chloro-1,3- | 40% | 498 | (DMSO-d6) 1.91-2.00 |
| | (methyl- | benzodioxol-4- | | [M + H]⁺ | (m, 2H), 2.91 (dd, |
| | enedioxy)- | yl)carbonyl]-O-[3- | | | 1H), 3.05 (dd, 1H), |
| | 6-chloro | (pyridin-2- | | | 3.35 (bs partially |
| | | ylamino)propyl]- | | | hidden by H2O, 2H), |
| | | L-tyrosine | | | 4.00 (t, 2H), 4.31-4.40 |
| | | | | | (m, 1H), 6.09 (d, 2H), |
| | | | | | 6.44 (dd, 1H), 6.46 (s, |
| | | | | | 1H), 6.55 (t, 1H), 6.81 |
| | | | | | (d, 2H), 6.90 (d, 1H), |
| | | | | | 6.94 (d, 1H), 7.16 (d, |
| | | | | | 2H), 7.34 (ddd, 1H), |
| | | | | | 7.95 (dd, 1H), 8.39 |
| | | | | | (bs, 1H) |
| 12.13 | 2-chloro- | N-(2-chloro-4- | 49% | 472 | (DMSO-d6 + 5 drops |
| | 4-fluoro | fluorobenzoyl)-O- | | [M + H]⁺ | TFAd) 2.04-2.14 (m, |
| | | [3-(pyridin-2- | | | 2H), 2.92 (dd, 1H), |
| | | ylamino)propyl]- | | | 3.13 (dd, 1H), 351 (t, |
| | | L-tyrosine | | | 2H), 4.08 (t, 2H), 4.58 |
| | | | | | (dd, 1H), 6.86 (dd, |
| | | | | | 1H), 6.89 (d, 2H), |
| | | | | | 7.09 (d, 1H), 7.24 (d, |
| | | | | | 2H), 7.27 (dd, 1H), |
| | | | | | 7.35 (dd, 1H), 7.42 |
| | | | | | (dd, 1H), 7.86-7.94 |
| | | | | | (m, 2H) |
| 12.14 | 2-bromo- | N-(2-bromo-6- | 31% | 531 | (DMSO-d6) 1.89-2.01 |
| | 6-chloro | chlorobenzoyl)-O- | | [M − H]⁻ | (m, 2H), 2.90 (dd, |
| | | [3-(pyridin-2- | | | 1H), 3.04 (dd, 1H), |
| | | ylamino)propyl]- | | | 3.33-3.37 (m partially |
| | | L-tyrosine | | | hidden by H2O, 2H), |
| | | | | | 4.00 (t, 2H), 4.48-4.56 |
| | | | | | (m, 1H), 6.44 (ddd, |
| | | | | | 1H), 6.45 (s, 1H), 6.82 |
| | | | | | (d, 2H), 7.19 (d, 2H), |
| | | | | | 7.31 (dd, 1H), 7.34 |
| | | | | | (ddd, 1H), 7.47 (dd, |
| | | | | | 1H), 7.58 (dd, 1H), |
| | | | | | 7.95 (dd, 1H), 8.77 |
| | | | | | (bs, 1H) |

The methyl O-[3-(pyridin-2-ylamino)propyl]-L-tyrosinate used as the starting material for Example 12 was prepared as follows:

DTAD (46.69 g, 203 mmol) was added to a mixture of methyl N-(tert-butoxycarbonyl)-L-tyrosinate (30 g, 102 mmol), 3-bromopropanol (13.9 ml, 153 mmol) and triphenylphosphine (59.4 g, 224 mmol) in DCM (100 ml). The mixture was stirred at room temperature for 2 hours. One further equivalent of all the reagents was added and the reaction mixture was stirred for 16 hours. After evaporation, the residue was purified by flash chromatography eluting with 20% ethyl acetate in petroleum ether to afford a waxy white solid which was suspended in heptane and collected by filtration. The filtrate was evaporated to dryness to give methyl O-(3-bromopropyl)-N-(tert-butoxycarbonyl)-L-tyrosinate (40 g, 95%); ¹H NMR Spectrum: (DMSO-d6) 1.33 (9H, s), 2.19-2.26 (2H, m), 2.77 (1H, dd), 2.91 (1H, dd), 3.60 (3H, s), 3.65 (2H, t), 4.04 (2H, t), 4.10 (1H, ddd), 6.86 (2H, d), 7.14 (2H, d,) 7.26 (1H, d); Mass Spectrum [M+H]⁺=417.

NaH (1.64 g, 40.9 mmol) and methyl O-(3-bromopropyl)-N-(tert-butoxycarbonyl)-L-tyrosinate (17 g, 40.9 mmol) were added dropwise over 5 minutes to a solution of tert-butyl pyridin-2-ylcarbamate (7.93 g, 40.9 mmol) in DMF (400 ml) at −10° C. The reaction mixture was allowed to warm to room temperature and stir for 2 hours. The reaction mixture was extracted with diethyl ether (11) and washed with saturated citric acid (100 ml) and water (3×100 ml), concentrated and the residue was purified by flash chromatography eluting with 20% diethyl ether in petroleum ether to afford methyl N-(tert-butoxycarbonyl)-O-{3-[(tert-butoxycarbonyl)(pyridin-2-yl)amino]propyl}-L-tyrosinate as a white foam (18.8 g, 87%); ¹H NMR Spectrum: (DMSO-d6) 1.32 (9H, s), 1.42 (9H, s), 1.94-2.01 (2H, m), 2.76 (1H, dd), 2.89 (1H, dd), 3.60 (3H, s), 3.94 (2H, t), 4.00 (2H, t), 4.09 (1H, ddd), 6.74-6.83 (2H, m), 7.08-7.15 (3H, m), 7.23 (1H, d), 7.56 (1H, d), 7.74 (1H, ddd), 8.35 (1H, dd); Mass Spectrum [M+H]⁺=530.

A solution of methyl N-(tert-butoxycarbonyl)-O-{3-[(tert-butoxycarbonyl)(pyridin-2-yl)amino]propyl}-L-tyrosinate (2 g, 3.78 mmol) in MeOH (50 ml) was treated with 4M HCl in dioxane (50 ml) at 0° C. The resulting solution was allowed to warm to room temperature and stirred for 16 hours. After evaporation to dryness, the residue was triturated with diethyl ether to afford methyl O-[3-(pyridin-2-ylamino)propyl]-L-tyrosinate a white solid, which was collected by filtration and dried under vacuum (1.35 g, 89%); ¹H NMR Spectrum: (DMSO-d6+TFAd) 2.04-2.10 (2H, m), 2.99-3.12 (2H, m), 3.51 (2H, t), 3.68 (3H, s), 4.07 (2H, t), 4.27 (1H, t), 6.84 (1H, dd), 6.90 (2H, d), 7.07 (1H, d), 7.13 (2H, d), 7.83-7.95 (2H, m); Mass Spectrum [M+H]⁺=330.

### Example 13

### N-(2,6-dichlorobenzoyl)-4-[4-(Pyridin-2-ylamino)but-1-yn-1-yl]-L-phenylalanine

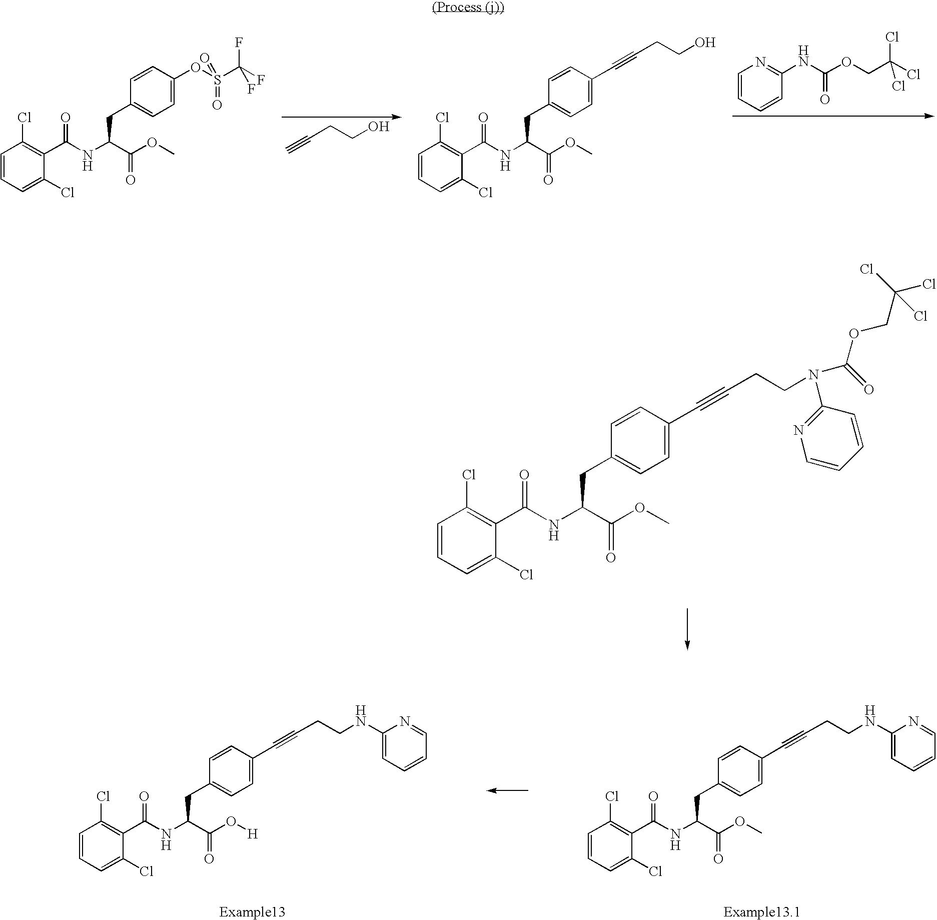

LiOH (110 mg) was added to a solution of methyl N-(2,6-dichlorobenzoyl)-4-[4-(pyridin-2-ylamino)but-1-yn-1-yl]-L-phenylalaninate (130 mg, 0.26 mmol) in DMA (1 ml), MeOH (0.1 ml) and H₂O (0.1 ml) was added. After stirring at room temperature for 2 hours, the reaction mixture was purified by C18 reverse phase chromatography (basic conditions) to give the title product as a white solid (68 mg, 54%); ¹H NMR Spectrum: (DMSO-d6) 2.64 (2H, t), 2.93 (1H, dd), 3.14 (1H, dd), 3.43-3.50 (2H, m), 4.62 (1H, dd), 6.48 (1H, ddd), 6.50 (1H, d), 6.73 (1H, t), 7.25 (2H, d), 7.28 (2H, d), 7.36 (1H, ddd), 7.39 (1H, dd), 7.43 (1H, s), 7.44 (1H, dd), 7.98 (1H, dd), 8.92 (1H, bs); Mass Spectrum [M+H]⁺=481.

The methyl N-(2,6-dichlorobenzoyl)-4-[4-(pyridin-2-ylamino)but-1-yn-1-yl]-L-phenylalaninate used as the starting material was prepared as follows:

3-Butyn-1-ol (1.51 ml, 20 mmol) was added to a solution of methyl N-(2,6-dichlorobenzoyl)-O-[(trifluoromethyl)sulfonyl]-L-tyrosinate (5 g, 10 mmol), CuI (76 mg, 0.4 mmol), triethylamine (5.58 ml, 40 mmol) and Pd(Cl₂)(PPh₃)₂ (421 mg, 0.6 mmol) in DMF (50 ml) under an argon atmosphere. The mixture was heated at 80° C. overnight. After evaporation, the residue was extracted with ethyl acetate and purified by silica gel flash chromatography eluting with 0 to 30% ethyl acetate in DCM to give methyl N-(2,6-dichlorobenzoyl)-4-(4-hydroxybut-1-yn-1-yl)-L-phenylalaninate as a foam (4.2 g, 76%); ¹H NMR Spectrum: (DMSO-d6) 2.54 (2H, t), 2.94 (1H, dd), 3.14 (1H, dd), 3.54-3.61 (2H, m), 3.65 (3H, s), 4.74 (1H, ddd), 4.89 (1H, t), 7.25 (2H, d), 7.30 (2H, d), 7.40 (1H, dd), 7.44 (1H, s), 7.45 (1H, dd), 9.20 (1H, d); Mass Spectrum [M+H]⁺=420.

Triphenylphosphine (2.15 g, 8.2 mmol) and DTAD (1.89 g, 8.2 mmol) were added to a solution of methyl N-(2,6-dichlorobenzoyl)-4-(4-hydroxybut-1-yn-1-yl)-L-phenylalaninate (2.3 g, 5.5 mmol) in THF (50 ml) at 0° C. The mixture was stirred at room temperature for 10 minutes and 2,2,2-trichloroethyl pyridin-2-ylcarbamate (670 mg, 2.5 mmol) was added. The reaction mixture was allowed to stir at room temperature for 24 hours, then was concentrated and purified by silica gel flash chromatography eluting with 0 to 40% ethyl acetate in petroleum ether to give methyl N-(2,6-dichlorobenzoyl)-4-(4-{pyridin-2-yl[(2,2,2-trichloroethoxy)carbonyl]amino}but-1-yn-1-yl)-L-phenylalaninate as a foam (0.61 g, 37%); ¹H NMR Spectrum: (DMSO-d6) 2.92 (2H, t), 2.93 (1H, dd), 3.13 (1H, dd), 3.65 (3H, s), 4.20 (2H, t), 4.73 (1H, ddd), 4.99 (2H, s), 7.16 (2H, d), 7.23 (2H, d), 7.26 (1H, ddd), 7.41 (1H, dd), 7.44 (1H, s), 7.45 (1H, dd), 7.66 (1H, d), 7.65 (1H, ddd), 8.48 (dd, 1H), 9.19 (1H, d).

Methyl N-(2,6-dichlorobenzoyl)-4-(4-{pyridin-2-yl[(2,2,2-trichloroethoxy)carbonyl]amino}but-1-yn-1-yl)-L-phenylalaninate (300 mg, 0.45 mmol) in solution in THF (1 ml), H₂O (0.5 ml) and AcOH (1 ml) was stirred overnight in the presence of zinc dust (88 mg, 1.34 mmol). Another amount of zinc (2 equivalents) and AcOH (1 ml) was added during the course of the reaction. After addition of ethyl acetate, the mixture was filtered. The filtrate was neutralised with NH₃ MeOH and purified by silica gel flash chromatography eluting with 0 to 40% ethyl acetate in DCM to give, as Example 13.1, methyl N-(2,6-dichlorobenzoyl)-4-[4-(pyridin-2-ylamino)but-1-yn-1-yl]-L-phenylalaninate as a foam (130 mg, 59%); ¹H NMR Spectrum: (DMSO-d6) 2.64 (2H, t), 2.94 (1H, dd), 3.14 (1H, dd), 3.45 (1H, d), 3.48 (1H, d), 3.66 (3H, s), 4.74 (1H, ddd), 6.46-6.52 (2H, m), 6.72 (1H, t), 7.25 (2H, d), 7.30 (2H, d), 7.36 (1H, ddd), 7.40 (1H, dd), 7.44 (1H, s), 7.45 (1H, dd), 7.98 (1H, dd), 9.20 (1H, d); Mass Spectrum [M+H]⁺=496.

### Example 14

### N-(2,6-dichlorobenzoyl)-3-{5-[2-(5,6,7,8-tetrahydro-1,8-naphthyridin-2-yl)ethoxy]pyridin-2-yl}-L-alanine

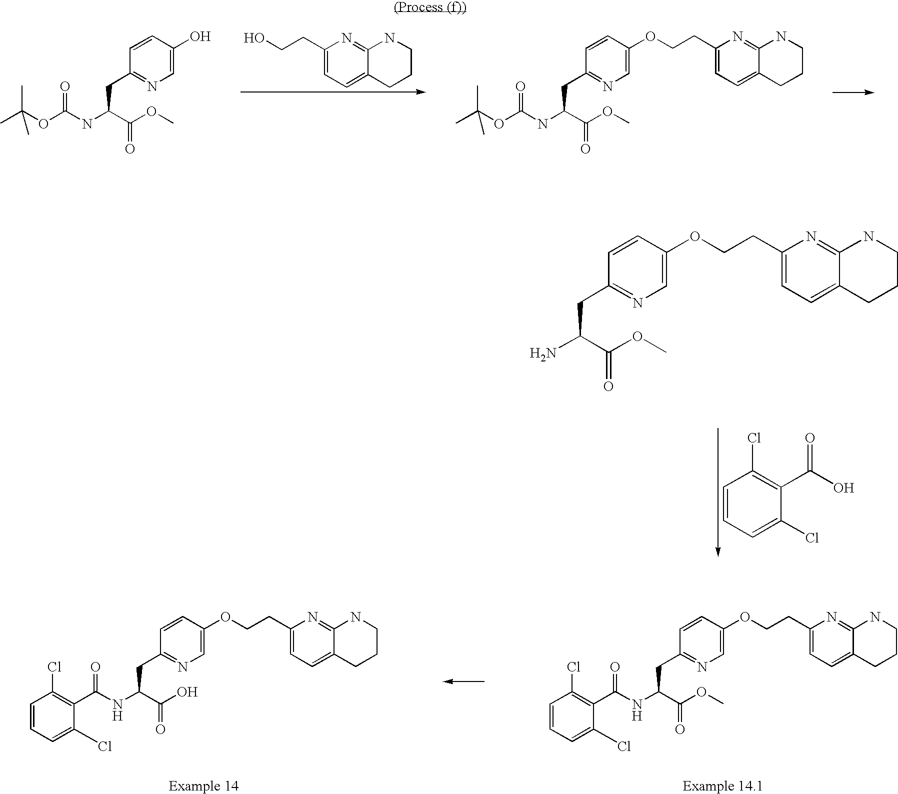

LiOH (21 mg, 0.49 mmol) was added to a solution of methyl N-(2,6-dichlorobenzoyl)-3-{5-[2-(5,6,7,8-tetrahydro-1,8-naphthyridin-2-yl)ethoxy]pyridin-2-yl}-L-alaninate (130 mg, 0.25 mmol) in DMA (1.2 ml). After stirring at room temperature overnight, the reaction mixture was purified by C18 reverse phase chromatography (basic conditions) to give the title compound as a white solid (50 mg, 39%); ¹H NMR Spectrum: (DMSO-d6): 1.71-1.78 (2H, m), 2.61 (2H, t), 2.88 (2H, t), 3.01 (1H, dd), 3.19 (1H, dd), 3.21-3.26 (2H, m), 4.27 (2H, t), 4.73-4.81 (1H, m), 6.35 (1H, s), 6.36 (1H, d), 7.06 (1H, d), 7.20 (1H, d), 7.28 (1H, dd), 7.37 (1H, dd), 7.40-7.44 (2H, m), 8.16 (1H, d), 8.81 (1H, d); Mass Spectrum [M+H]⁺=515.

The methyl N-(2,6-dichlorobenzoyl)-3-{5-[2-(5,6,7,8-tetrahydro-1,8-naphthyridin-2-yl)ethoxy]pyridin-2-yl}-L-alaninate used as the starting material was prepared as follows:

Triphenylphosphine (495 mg, 1.89 mmol), 2-(5,6,7,8-tetrahydro-1,8-naphthyridin-2-yl)ethanol (337 mg, 1.89 mmol) and DTAD (435 mg, 1.89 mmol) were added to a suspension of methyl N-(tert-butoxycarbonyl)-3-(5-hydroxypyridin-2-yl)-L-alaninate (374 mg, 1.26 mmol) in DCM (3 ml) under an argon atmosphere. The mixture was stirred at 5° C. for one hour and at room temperature for 3 hours. After evaporation, the residue was purified by silica gel flash chromatography eluting with 30 to 50% ethyl acetate in DCM to give methyl N-(tert-butoxycarbonyl)-3-{5-[2-(5,6,7,8-tetrahydro-1,8-naphthyridin-2-yl)ethoxy]pyridin-2-yl}-L-alaninate as a foam (0.61 g, 37%); ¹H NMR Spectrum: (DMSO-d6) 1.32 (9H, s), 1.70-1.78 (2H, m), 2.61 (2H, t), 2.88 (2H, t), 2.94 (1H, dd), 3.02 (1H, dd), 3.20-3.26 (2H, m), 3.59 (3H, s), 4.27 (2H, t), 4.36 (1H, ddd), 6.33 (1H, bs), 6.36 (1H, d), 7.05 (1H, d), 7.17 (1H, d), 7.23 (1H, d), 7.30 (1H, dd), 8.17 (1H, d).

TFA (1 ml) was added to a solution of methyl N-(tert-butoxycarbonyl)-3-{5-[2-(5,6,7,8-tetrahydro-1,8-naphthyridin-2-yl)ethoxy]pyridin-2-yl}-L-alaninate (533 mg, 1.17 mmol) in DCM (2 ml). The mixture was stirred at room temperature overnight. After evaporation, the residue was dissolved in NH₃/MeOH 7N and evaporated. Dissolution in DCM was followed by filtration of the salts and a purification by silica gel flash chromatography eluting with 5-20% MeOH in DCM to afford methyl 3-{5-[2-(5,6,7,8-tetrahydro-1,8-naphthyridin-2-yl)ethoxy]pyridin-2-yl}-L-alaninate as a foam (quantitative yield); ¹H NMR Spectrum: (DMSO-d6) 1.70-1.79 (2H, m), 2.61 (2H, t), 2.88 (2H, t), 2.94 (1H, dd), 3.03 (1H, dd), 3.20-3.26 (2H, m), 3.60 (3H, s), 4.16-4.23 (1H, m), 4.28 (2H, t), 4.31 (2H, bs), 6.32 (1H, bs), 6.36 (1H, d), 7.06 (1H, d), 7.17 (1H, d), 7.31 (1H, dd), 8.15 (1H, d).

Methyl 3-{5-[2-(5,6,7,8-tetrahydro-1,8-naphthyridin-2-yl)ethoxy]pyridin-2-yl}-L-alaninate (200 mg, 0.56 mmol), HOPO (62 mg, 0.56 mmol) and EDCI (119 mg, 0.62 mmol) were added to a suspension of 2-6-dichlorobenzoic acid (107 mg, 0.56 mmol) in DCM (3 ml). The mixture was stirred at 0° C. for one hour and at room temperature for 4 hours. After extraction with DCM and evaporation, the residue was purified by silica gel flash chromatography eluting with 5% MeOH in DCM to afford, as Example 14.1, methyl N-(2,6-dichlorobenzoyl)-3-{5-[2-(5,6,7,8-tetrahydro-1,8-naphthyridin-2-yl)ethoxy]pyridin-2-yl}-L-alaninate as a white solid (168 mg, 57%); ¹H NMR Spectrum: (DMSO-d6) 1.71-1.79 (2H, m), 2.61 (2H, t), 2.88 (2H, t), 3.03 (1H, dd), 3.19 (1H, dd), 3.20-3.27 (2H, m), 3.65 (3H, s), 4.28 (2H, t), 4.93 (1H, ddd), 6.34 (1H, bs), 6.36 (1H, d), 7.06 (1H, d), 7.20 (1H, d), 7.30 (1H, dd), 7.39 (1H, d), 7.40-7.44 (2H, m), 8.18 (1H, d), 9.14 (1H, d); Mass Spectrum [M+H]⁺=529.

### Example 15

### N-(2,6-dichlorobenzoyl)-4-[4-(Pyridin-2-ylamino)piperidin-1-yl]-L-phenylalanine

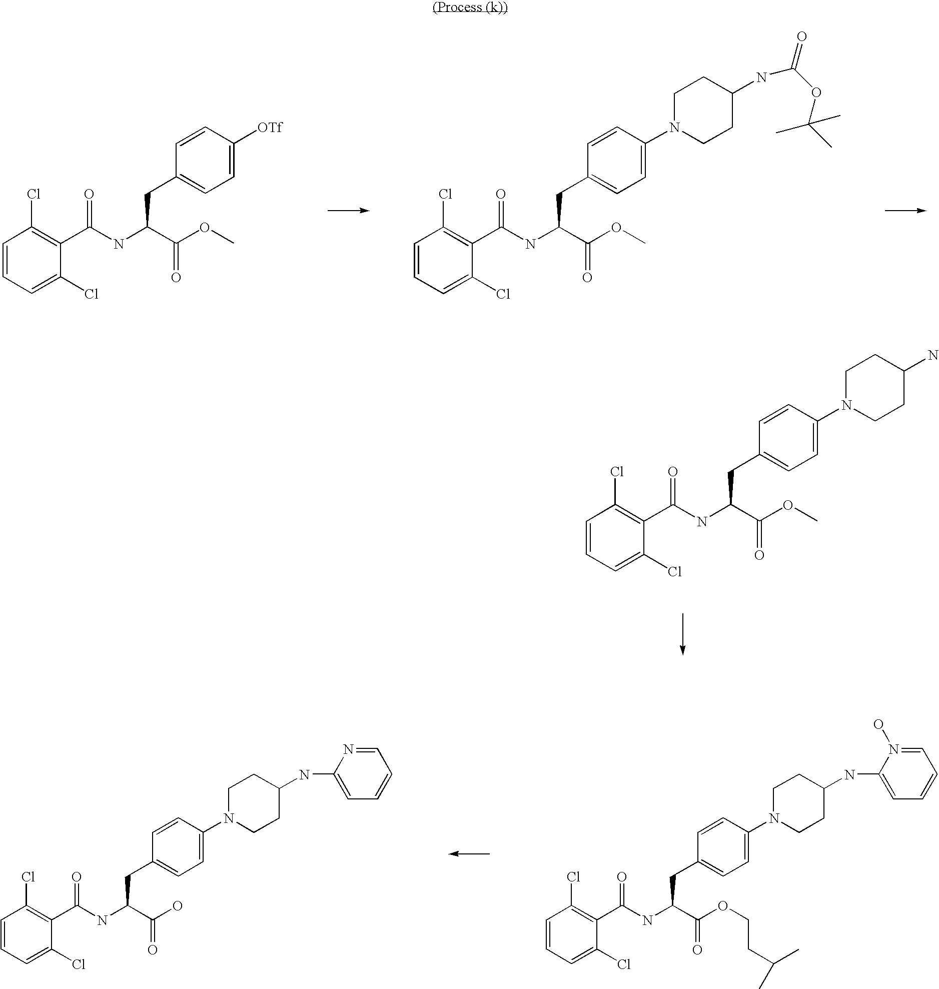

PCl₃ (0.08 ml, 0.92 mmol) was added dropwise to a solution of 3-methylbutyl N-(2,6-dichlorobenzoyl)-4-{4-[(1-oxidopyridin-2-yl)amino]piperidin-1-yl}-L-phenylalaninate (50 mg) in chloroform (0.6 ml) at −20° C. The mixture was heated at 60° C. for 2 hours. After addition of ice and careful neutralization with NaOH (pH 8), the mixture was extracted with methylene chloride. The solvent was removed and the residue was dissolved in DMA (0.5 ml)/H₂O (0.1 ml) and treated with NaOH 5N (0.2 ml) at 600 for 1 hour. The crude reaction was purified by C18 reverse phase chromatography (basic conditions) to give the title product as a white solid (15 mg, 30%); ¹H NMR Spectrum: (DMSO-d6) 1.44-1.56 (2H, m), 1.93-2.01 (2H, m), 2.72-2.81 (2H, m), 2.86 (1H, dd), 3.02 (1H, dd), 3.60 (2H, d), 3.79-3.88 (1H, m), 3.48 (1H, dd), 6.39-6.49 (3H, m), 6.84 (2H, d), 7.12 (2H, d), 7.33 (1H, ddd), 7.38 (1H, dd), 7.43 (1H, s), 7.44 (1H, dd), 7.95 (1H, dd), 8.66 (1H, bs); Mass Spectrum [M+H]⁺=513.

The 3-methylbutyl N-(2,6-dichlorobenzoyl)-4-{4-[(1-oxidopyridin-2-yl)amino]piperidin-1-yl}-L-phenylalaninate used as the starting material was prepared as follows:

tert-butyl piperidin-4-ylcarbamate (835 mg, 4 mmol) was added under argon atmosphere to a degassed solution of methyl N-(2,6-dichlorobenzoyl)-O-[(trifluoromethyl)sulfonyl]-L-tyrosinate (1 g, 2 mmol), bis(dibenzylideneacetone) palladium(0) (230 mg, 0.4 mmol), 9,9-dimethyl-4,5-bis(diphenylphosphino)xanthene (230 mg, 0.4 mmol) and caesium carbonate (1.3 g, 4 mmol) in toluene (10 ml). The reaction mixture was heated at 100° C. for 10 hours then cooled down to room temperature and concentrated in vacuo. The residue was partitioned between water and ethyl acetate to give after concentration an oil that was purified by flash chromatography using 10-30% ethyl acetate in DCM to afford methyl 4-{4-[(tert-butoxycarbonyl)amino]piperidin-1-yl}-N-(2,6-dichlorobenzoyl)-L-phenylalaninate as an solid (345 mg, 31%); ¹H NMR Spectrum: (CDCl₃) 1.45 (9H, s), 1.51 (2H, ddd), 2.03 (2H, d), 2.73-2.84 (2H, m), 3.14-3.24 (2H, m), 3.52-3.59 (2H, m), 3.60 (1H, bs), 3.76 (3H, s), 4.47 (1H, d), 5.14 (1H, ddd), 6.24 (1H, d), 6.83 (2H, d), 7.07 (2H, d), 7.26 (1H, dd partially hidden by CHCl3), 7.30 (1H, s), 7.31 (1H, dd).

Methyl 4-{4-[(tert-butoxycarbonyl)amino]piperidin-1-yl}-N-(2,6-dichlorobenzoyl)-L-phenylalaninate (340 mg, 0.62 mmol) in solution in DCM (3 ml) was treated with TFA (3.523 ml). The solution was stirred at room temperature for 2 hours. The solvent was removed and the residue was dissolved in DCM/NH₃/MeOH 7N. The reaction mixture was purified by silica gel flash chromatography eluting with 5% MeOH in DCM to give methyl 4-(4-aminopiperidin-1-yl)-N-(2,6-dichlorobenzoyl)-L-phenylalaninate as a white foam (0.26 g, 93%).

2-chloropyridine N-oxide (46 mg, 0.28 mmol) was added to a solution of methyl 4-(4-aminopiperidin-1-yl)-N-(2,6-dichlorobenzoyl)-L-phenylalaninate (0.125 g, 0.28 mmol) in 3-methyl-1-butanol (2 ml), sodium carbonate (0.12 g, 1.11 mmol), potassium iodide (5 mg, 0.03 mmol). The mixture was heated at 130° C. under argon for 24 hours and purified by C18 reverse phase chromatography (basic conditions) to give 3-methylbutyl N-(2,6-dichlorobenzoyl)-4-{4-[(1-oxidopyridin-2-yl)amino]piperidin-1-yl}-L-phenylalaninate (50 mg, 29%); ¹H NMR Spectrum: (DMSO-d6) 0.86 (3H, d), 0.87 (3H, d), 1.39-1.46 (2H, m), 1.57-1.71 (3H, m), 1.94-2.00 (2H, m), 2.78-2.85 (2H, m), 2.86 (1H, dd), 3.00 (1H, dd), 3.57-3.66 (3H, m), 4.03-4.11 (2H, m), 4.65 (1H, ddd), 6.59-6.64 (1H, m), 6.88 (2H, d), 6.94 (1H, bs), 6.96 (1H, bs), 7.11 (2H, d), 7.20-7.25 (1H, m), 7.40 (1H, dd), 7.45 (1H, s), 7.46 (1H, dd), 8.12 (1H, dd), 9.16 (1H, d); Mass Spectrum [M+H]⁺=599.

### Example 16

The compound of Example 16.1 described below and the compounds shown in Table 4 were prepared using the following reaction scheme:

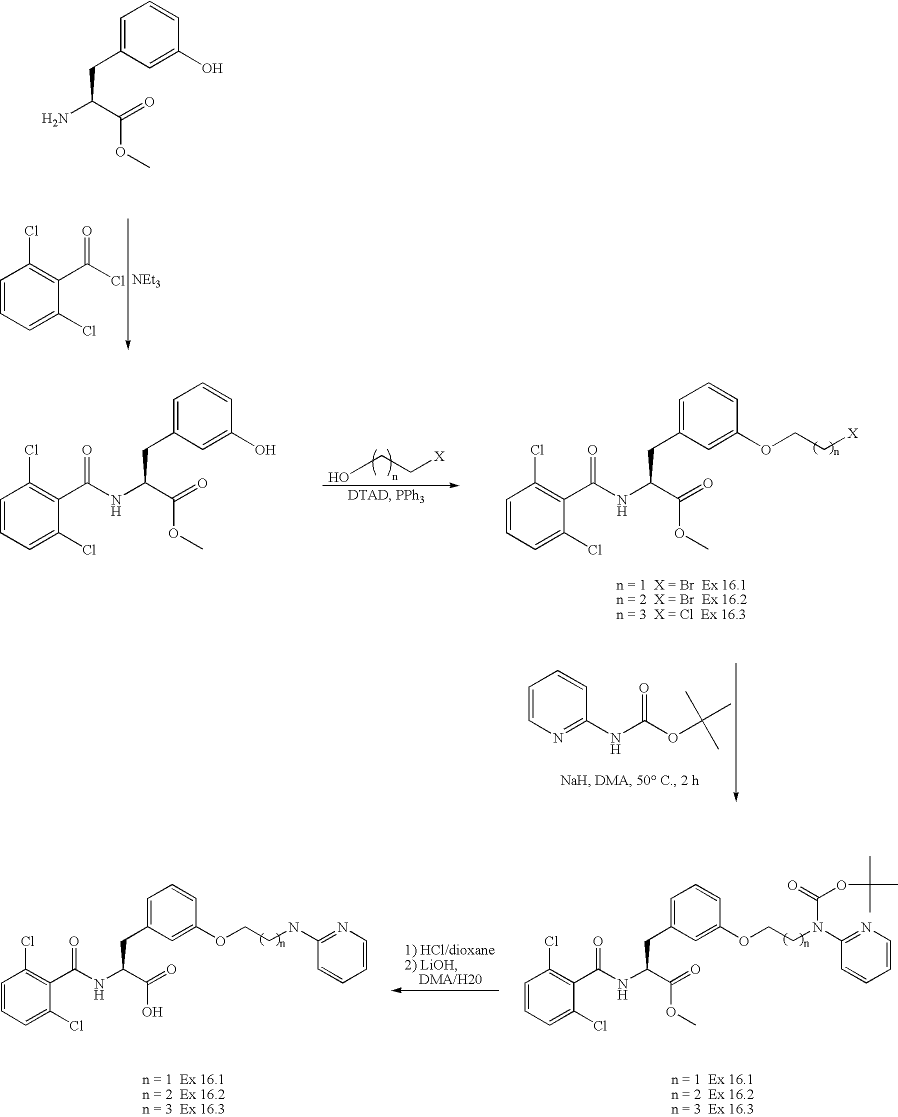

### Example 16.1

### N-(2,6-dichlorobenzoyl)-3-[2-(pyridin-2-ylamino)ethoxy]-L-phenylalanine

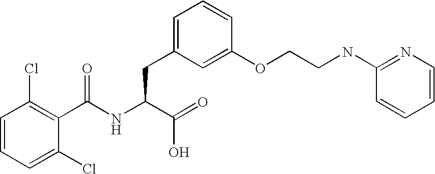

To a solution of methyl 3-{2-[(-tert-butoxycarbonyl)(pyridin-2-yl)amino]ethoxy}-N-(2,6-dichlorobenzoyl)-L-phenylalaninate (42 mg, 0.07 mmol) in MeOH (1 ml) was added a solution of HCl 4M in dioxane (53 μl, 0.21 mmol). The reaction mixture was heated at 50° C. for 3 hours to reach completion. NH3/MeOH 7N (1 ml) was added, then the solution was filtered and solvents were evaporated. The residue was dissolved in DMA/H2O (0.7 ml/0.5 ml) and LiOH (20 mg) was added. Reaction was completed after 3 hours at room temperature and 1 hour at 40° C. The crude reaction was directly purified by C18 reverse phase chromatography (basic conditions) to afford the title compound as a white solid (20 mgs, 59% over 2 steps); Mass Spectrum [M+H]⁺=474; ¹H NMR (500 MHz): (DMSOd6) 2.89 (dd, 1H), 3.09 (dd, 1H), 3.58-3.65 (m, 2H), 4.05 (t, 2H), 4.60-4.70 (m, 1H), 6.48 (ddd, 1H), 6.52 (d, 1H), 6.70 (t, 2H), 6.80 (dd, 1H), 6.86 (d, 1H), 6.90 (bs, 1H), 7.17 (t, 1H), 7.33-7.44 (m, 4H), 7.98 (dd, 1H), 9.04 (bs, 1H).

### Examples 16.2 and 16.3

The procedure described above for example 16.1 was repeated using the appropriate methyl ester N-BOC precursor. Thus were obtained the compounds shown in

TABLE 4

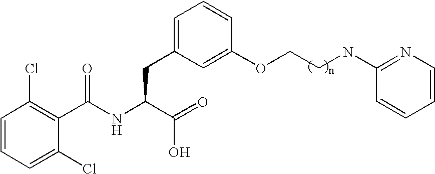

| | | | | Mass | ¹H NMR Data |
|---|---|---|---|---|---|
| Example | n | Name | Yield | Ion | (500 MHz) |
| 16.2 | 2 | N-(2,6- | 52% | 488 | (DMSO-d6) 1.89-2.02 |
| | | dichlorobenzoyl)- | | [M + H]⁺ | (m, 2H), 2.92 (dd, 1H), |
| | | 3-[3-(pyridin-2- | | | 3.10 (dd, 1H), 3.32-3.39 |
| | | ylamino)propoxy]- | | | (m, 2H), 4.02 (t, 2H), |
| | | L-phenylalanine | | | 4.51-4.62 (m, 1H), 6.39- |
| | | | | | 6.50 (m, 2H), 6.56 (bs, |
| | | | | | 1H), 6.75 (dd, 1H), 6.85 |
| | | | | | (d, 1H), 6.88 (s, 1H), |
| | | | | | 7.15 (t, 1H), 7.30-7.46 |
| | | | | | (m, 4H), 7.95 (d, 1H), |
| | | | | | 8.77 (bs, 1H) |
| 16.3 | 3 | N-(2,6- | 37% | 500 | (DMSO-d6) 1.60-1.70 |
| | | dichlorobenzoyl)- | | [M − H]⁻ | (m, 2H), 1.71-1.81 (m, |
| | | 3-[4-(pyridin-2- | | | 2H), 2.89 (dd, 1H), 3.10 |
| | | ylamino)butoxy]- | | | (dd, 1H), 3.23-3.31 (m |
| | | L-phenylalanine | | | partially hidden by H2O, |
| | | | | | 2H), 3.95 (t, 2H), 4.59- |
| | | | | | 4.68 (m, 1H), 6.40-6.47 |
| | | | | | (m, 2H), 6.47 (t, 1H), |
| | | | | | 6.75 (dd, 1H), 6.81-6.90 |
| | | | | | (m, 2H), 7.16 (t, 1H), |
| | | | | | 7.29-7.46 (m, 4H), 7.94 |
| | | | | | (d, 1H), 8.98 (bs, 1H) |

The starting materials used in Examples 16.1-16.3 were prepared as follows:

Methyl L-m-tyrosinate (163 mg, 0.83 mmol) was suspended in dichloromethane (3 ml) at 4° C. and triethylamine (0.17 ml, 1.25 mmol) was added as a single portion. 2,6-dichlorobenzoyl chloride (0.13 ml, 0.92 mmol) was added dropwise (T<10° C.) and the reaction mixture stirred at room temperature for 2 hours. The solution was washed with water and brine. The organic layer was concentrated and purified by silica gel flash chromatography (0 to 20% ethyl acetate in petroleum ether) to give methyl N-(2,6-dichlorobenzoyl)-3-hydroxy-L-phenylalaninate as a white solid (170 mg, 55%); Mass Spectrum [M+H]⁺=368; ¹H NMR (500 MHz): (DMSO-d6) 2.85 (dd, 1H), 3.03 (dd, 1H), 3.65 (s, 3H), 4.68 (ddd, 1H), 6.60 (dd, 1H), 6.66 (dd, 1H), 6.68 (d, 1H), 7.06 (t, 1H), 7.40 (dd, 1H), 7.44 (s, 1H), 7.46 (d, 1H), 9.18 (d, 1H), 9.26 (s, 1H).

To a solution of methyl N-(2,6-dichlorobenzoyl)-3-hydroxy-L-phenylalaninate (200 mg, 0.54 mmol) in dichloromethane (1 ml) at room temperature were added triphenylphosphine (312 mg, 1.19 mmol) and 2-bromoethanol (57 μl, 0.81 mmol). DTAD was then added (251 mg, 1.09 mmol). The reaction mixture was allowed to stir at room temperature overnight, and was then concentrated and purified by silica gel flash chromatography (0 to 40% ethyl acetate in petroleum ether) to give methyl 3-(2-bromoethoxy)-N-(2,6-dichlorobenzoyl)-L-phenylalaninate as a white solid (142 mg, 55%).

The procedure described above for the preparation of methyl 3-(2-bromoethoxy)-N-(2,6-dichlorobenzoyl)-L-phenylalaninate was repeated using the appropriate bromo or chloro commercially available alcohol. Thus were obtained the other compounds described in Table 4.1:

TABLE 4.1

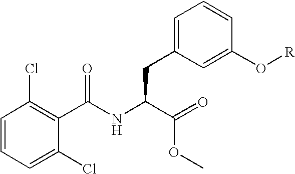

| | | | | Mass | ¹H NMR Data |
|---|---|---|---|---|---|
| Compound | R | Name | yield | Ion | (500 MHz) |
| 4.1a | 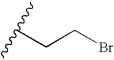 | methyl 3-(2- bromoethoxy)- N-(2,6- dichlorobenzoyl)- L-phenyl- alaninate | 55% | 475 [M + H]⁺ | (DMSO-d6) 2.92 (dd, 1H), 3.12 (dd, 1H), 3.66 (s, 3H), 3.78 (t, 2H), 4.29 (t, 2H), 4.74 (ddd, 1H), 6.80 (dd, 1H), 6.88 (d, 1H), 6.91 (bs, 1H), 7.20 (t, 1H), 7.40 (dd, 1H), |
| # 7.43-7.47 (m, 2H), 9.19 (d, 1H) | | | | | |
| 4.1b | 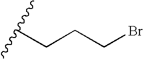 | methyl 3-(3- bromopropoxy)- N-(2,6- dichlorobenzoyl)- L-phenyl- alaninate | 58% | 489 [M + H]⁺ | |
| 4.1c | 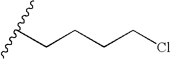 | methyl 3-(4- chlorobutoxy)- N-(2,6- dichlorobenzoyl)- L-phenyl- alaninate | 39% | — | (DMSO-d6) 1.78- 1.93 (m, 4H), 2.91 (dd, 1H), 3.10 (dd, 1H), 3.66 (s, 3H), 3.70 (t, 2H), 3.97 (t, 2H), 4.73 (dd, 1H), 6.77 (dd, 1H), 6.84 (d, 1H), 6.88 (s, 1H), 7.18 (t, 1H), 7.40 (dd, 1H), |
| # 7.43-7.47 (m, 2H), 9.18 (d, 1H) | | | | | |

To a solution of tert-butyl pyridin-2-ylcarbamate (66 mg, 0.34 mmol) in DMA (1 ml) was added sodium hydride (60% in oil, 14 mg, 0.34 mmol) at 0° C. The temperature was raised to room temperature for 15 minutes, then methyl 3-(2-bromoethoxy)-N-(2,6-dichlorobenzoyl)-L-phenylalaninate was added (135 mg, 0.28 mmol) and the reaction mixture was stirred at room temperature for 5 hours. A few drops of water were added, then solvents were evaporated, the residue was dissolved in dichloromethane and was purified by silica gel flash chromatography (0 to 50% Ethyl acetate in petroleum ether) to give methyl 3-{2-[(-tert-butoxycarbonyl)(pyridin-2-yl)amino]ethoxy}-N-(2,6-dichlorobenzoyl)-L-phenylalaninate as a white solid (45 mg, 27%).

The procedure described above for the preparation of methyl 3-{2-[(-tert-butoxycarbonyl)(pyridin-2-yl)amino]ethoxy}-N-(2,6-dichlorobenzoyl)-L-phenylalaninate was repeated using the appropriate bromo or chloro precursor. Thus were obtained the compounds described in Table 4.2:

TABLE 4.2

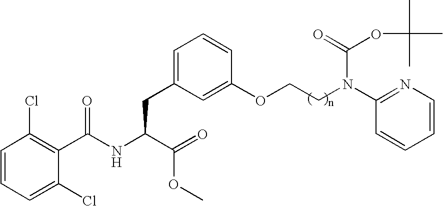

| | | | | Mass | ¹H NMR Data |
|---|---|---|---|---|---|
| Compound | n | Name | yield | Ion | (500 MHz) |
| 4.2a | 1 | methyl 3-{2- [(-tert- butoxycarbonyl) (pyridin-2- yl)amino]ethoxy}- N-(2,6- dichlorobenzoyl)- L- phenylalaninate | 27% | 588 [M + H]⁺ | (DMSO-d6) 1.45 (s, 9H), 2.89 (dd, 1H), 3.08 (dd, 1H), 3.65 (s, 3H), 4.15 (t, 2H), 4.20 (t, 2H), 4.71 (ddd, 1H), 6.71 (dd, 1H), |
| #6.81 (bs, 1H), 6.83 (d, 1H), 7.13-7.18 (m, 2H), 7.38-7.45 (m, 3H), 7.57 (d, 1H), 7.77 (ddd, 1H), 8.39 (ddd, 1H), 9.17 (d, 1H) | | | | | |
| 4.2b | 2 | methyl 3{3- [(-tert butoxycarbonyl) (pyridin-2- yl)amino]propoxy}- N-(2,6- dichlorobenzoyl)- L- phenylalaninate | 49% | 600 [M − H]⁻ | (DMSO-d6) 1.43 (s, 9H), 1.93-2.00 (m, 2H), 2.90 (dd, 1H), 3.08 (dd, 1H), 3.65 (s, 3H), 3.96 (t, 2H), 4.01 (t, 2H), 4.71 (ddd, 1H), |
| # 6.71 (dd, 1H), 6.79- 6.85 (m, 2H), 7.13 (ddd, 1H), 7.16 (t, 1H), 7.40 (dd, 1H), 7.42-7.46 (m, 2H), 7.57 (d, 1H), 7.75 (ddd, 1H), 8.37 (ddd, 1H), 9.19 (d, 1H) | | | | | |
| 4.2c | 3 | methyl 3{4- [(-tert butoxycarbonyl) (pyridin-2- yl)amino]butoxy}- N-(2,6- dichlorobenzoyl)- L- phenylalaninate | 21% | 616 [M + H]⁺ | (DMSO-d6) 1.44 (s, 9H), 1.64-1.73 (m, 4H), 2.90 (dd, 1H), 3.09 (dd, 1H), 3.65 (s, 3H), 3.85-3.97 (m, 4H), 4.71 (ddd, 1H), 6.73 (dd, 1H), |
| #6.80-6.86 (m, 2H), 7.11-7.20 (m, 2H), 7.36-7.46 (m, 3 H), 7.54 (d, 1H), 7.76 (ddd, 1H), 8.38 (d, 1H), 9.18 (d, 1H) | | | | | |

### Example 17

### N-(2,6-dichlorobenzoyl)-O-[2-(1-methyl-1,2,3,4-tetrahydropyrido[2,3-b]pyrazin-6-yl)ethyl]-L-tyrosine

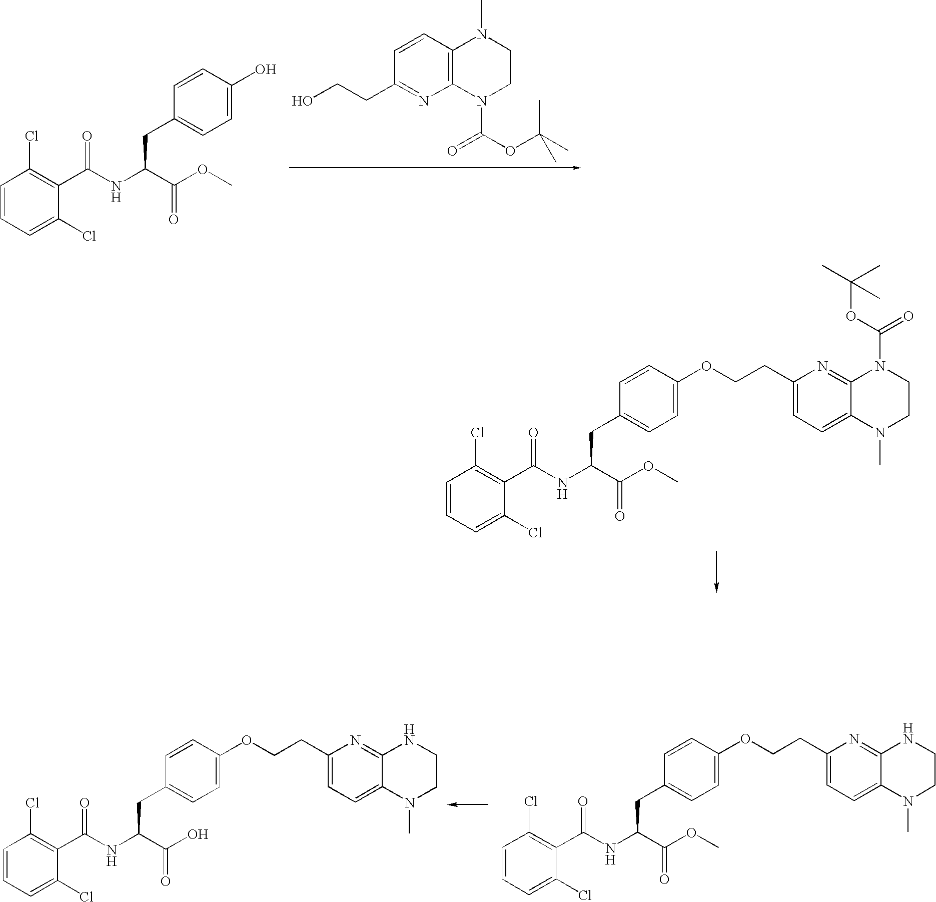

LiOH (47 mg, 1.13 mmol) was added to a solution of methyl N-(2,6-dichlorobenzoyl)-O-[2-(1-methyl-1,2,3,4-tetrahydropyrido[2,3-b]pyrazin-6-yl)ethyl]-L-tyrosinate (see Example 17.1 below) (210 mg, 0.38 mmol) in DMA/H₂O (2 ml/0.3 ml). After 4.5 hours at room temperature, the crude reaction mixture was purified by C18 reverse phase chromatography (basic conditions) to afford the title compound as a white solid (130 mg, 65%); Mass Spectrum [M+H]⁺=529; ¹H NMR Spectrum: (DMSOd6) 2.72 (s, 3H), 2.81 (t, 2H), 2.87 (dd, 1H), 3.01-3.09 (m, 3H), 3.40 (m, 2H), 4.14 (t, 2H), 4.55 (ddd, 1H), 6.35 (d, 1H), 6.43 (bs, 1H), 6.55 (d, 1H), 6.80 (d, 2H), 7.17 (d, 2H), 7.38 (dd, 1H), 7.41-7.46 (m, 2H), 8.87 (d, 1H).

The methyl N-(2,6-dichlorobenzoyl)-O-[2-(1-methyl-1,2,3,4-tetrahydropyrido[2,3-b]pyrazin-6-yl)ethyl]-L-tyrosinate (Example 17.1) used as the starting material was prepared as follows:

To a solution of methyl N-(2,6-dichlorobenzoyl)-L-tyrosinate (300 mg, 0.81 mmol) in dichloromethane (15 ml) were added tert-butyl 6-(2-hydroxyethyl)-1-methyl-2,3-dihydropyrido[2,3-b]pyrazine-4(1H)-carboxylate (358 mg, 1.22 mmol) described in WO 2004/058254 and triphenylphosphine (470 mg, 1.79 mmol) at room temperature. Then DTAD (394 mg, 1.71 mmol) was added portionwise over 5 minutes. The reaction mixture was allowed to stir at room temperature for 8 hours. After concentration, a purification by silica gel flash chromatography (30 to 70% ethyl acetate in petroleum ether) afforded tert-butyl 6-[2-(4-{(2S)-2-[(2,6-dichlorobenzoyl)amino]-3-methoxy-3-oxopropyl}phenoxy)ethyl]-1-methyl-2,3-dihydropyrido[2,3-b]pyrazine-4(1H)-carboxylate as a white foam (391 mg, 75%); Mass Spectrum [M+H]⁺=643; ¹H NMR Spectrum: (CDCl3) 1.49 (s, 9H), 2.92 (s, 3H), 3.09 (t, 2H), 3.18 (dd, 1H), 3.22 (dd, 1H), 3.31 (t, 2H), 3.74 (s, 3H), 3.84 (t, 2H), 4.26 (t, 2H), 5.14 (ddd, 1H), 6.25 (d, 1H), 6.82 (d, 2H), 6.84 (d, 1H), 6.90 (d, 1H), 7.09 (d, 2H), 7.26 (dd, 1H), 7.29-7.33 (m, 2H).

TFA (5 ml) was added to a solution of tert-butyl 6-[2-(4-{(2S)-2-[(2,6-dichlorobenzoyl)amino]-3-methoxy-3-oxopropyl}phenoxy)ethyl]-1-methyl-2,3-dihydropyrido[2,3-b]pyrazine-4(1H)-carboxylate (350 mg, 0.54 mmol) in dichloromethane (1 ml). The reaction mixture was stirred 4 hours at room temperature, then solvent was removed. The residue was dissolved in dichloromethane and NH₃/MeOH 7N was added. Filtration and evaporation were followed by a purification by silica gel flash chromatography (2 to 4% NH₃/MeOH 7N in dichloromethane) to afford methyl N-(2,6-dichlorobenzoyl)-O-[2-(1-methyl-1,2,3,4-tetrahydropyrido[2,3-b]pyrazin-6-yl)ethyl]-L-tyrosinate (Example 17.1) as a beige foam (269 mg, 90%); Mass Spectrum [M+H]⁺=543; ¹H NMR Spectrum: (CDCl3) 2.82 (s, 3H), 2.96 (t, 2H), 3.14-3.26 (m, 4H), 3.56-3.61 (m, 2H), 3.74 (s, 3H), 4.20 (t, 2H), 5.00 (bs, 1H), 5.14 (ddd, 1H), 6.27 (d, 1H), 6.46 (d, 1H), 6.58 (d, 1H), 6.82 (d, 2H), 7.08 (d, 2H), 7.25 (dd, 1H), 7.28-7.34 (m, 2H).

### Example 18

### N-(2,6-dichlorobenzoyl)-4-{3-[6-(methylamino)pyridin-2-yl]propyl}-L-phenylalanine

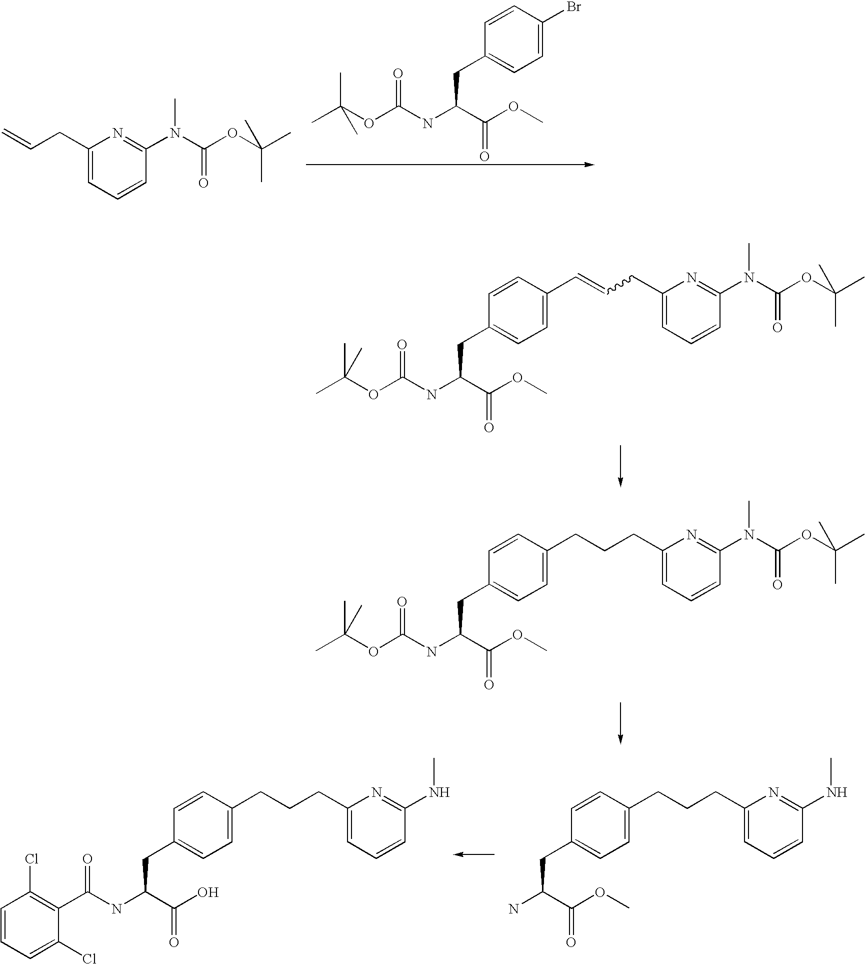

To a solution of methyl 4-{3-[6-(methylamino)pyridin-2-yl]propyl}-L-phenylalaninate dihydrochloride (280 mg, 0.69 mmol) in DMF (1 ml) were added N-Methyl morpholine (0.3 ml, 2.77 mmol) and 2,6-dichlorobenzoic acid (199 mg, 1.04 mmol). Then TBTU (395 mg, 1.04 mmol) was added in one portion and the reaction mixture was allowed to stir at room temperature overnight. LiOH (290 mg, 6.93 mmol) and water (0.5 ml) were added. After 2 hours at room temperature, the crude mixture was filtered and purified by C18 reverse phase chromatography (basic conditions) to afford the title compound as a white solid (56 mg, 17%).

The procedure described above was repeated using the appropriate commercially available acids. Thus were obtained the compounds described below in Table 5:

TABLE 5

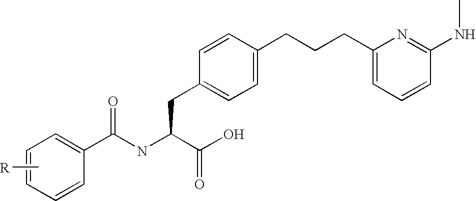

| | | | | Mass | ¹H NMR Data |
|---|---|---|---|---|---|
| Example | R | Name | Yield | Ion | (500 MHz) |
| 18.1 | 2,6- | N-(2,6- | 17% | 486 | (DMSO-d6) 1.84-1.94 |
| | dichloro | dichlorobenzoyl)- | | [M + H]⁺ | (m, 2H), 2.55 (t, 2H), |
| | | 4-{3-[6- | | | 2.74 (d, 3H), 2.92 (dd, |
| | | (methylamino) | | | 1H), 3.33 (m, 1H), 1.86- |
| | | pyridin-2- | | | 1.95 (m partially hidden |
| | | yl]propyl}-L- | | | by H2O, 2H), 4.50 (bs, |
| | | phenylalanine | | | 1H), 6.21 (d, 1H), 6.28 |
| | | | | | (q, 1H), 6.32 (d, 1H), |
| | | | | | 7.08 (d, 2H), 7.18 (d, |
| | | | | | 2H), 7.27 (dd, 1H), 7.37 |
| | | | | | (dd, 1H), 7.40-7.44 (m, |
| | | | | | 2H), 8.65 (bs, 1H) |
| 18.2 | 2-chloro | N-(2- | 54% | 452 | (DMSOd6) 1.85-1.94 |
| | | chlorobenzoyl)- | | [M + H]⁺ | (m, 2H), 2.48-2.53 (m |
| | | 4-{3-[6- | | | partially hidden by |
| | | (methylamino) | | | DMSOd5, 2H), 3.56 (t, |
| | | pyridin-2- | | | 2H), 2.73 (d, 3H), 2.93 |
| | | yl]propyl}-L- | | | (dd, 1H), 3.15 (dd, 1H), |
| | | phenylalanine | | | 4.48 (bs, 1H), 6.21 (d, |
| | | | | | 1H), 6.28 (q, 1H), 6.21 |
| | | | | | (d, 1H), 7.10 (d, 2H), |
| | | | | | 7.18 (d, 2H), 7.22 (d, |
| | | | | | 1H), 7.27 (dd, 1H), 7.33 |
| | | | | | (ddd, 1H), 7.40 (ddd, |
| | | | | | 1H), 7.43 (dd, 1H), 8.44 |
| | | | | | (bs, 1H) |
| 18.3 | 2- | N-(2-chloro-6- | 55% | 470 | (DMSOd6) 1.83-1.95 |
| | chloro- | fluorobenzoyl)- | | [M + H]⁺ | (m, 2H), 2.51 (t partially |
| | 6-fluoro | 4-{3-[6- | | | hidden by DMSOd5, 2H), |
| | | (methylamino) | | | 2.56 (t, 2H), 2.74 (d, 3H), |
| | | pyridin-2- | | | 2.90 (dd, 1H), 3.12 (dd, |
| | | yl]propyl}-L- | | | 1H), 4.57 (ddd, 1H), 6.22 |
| | | phenylalanine | | | (bs, 1H), 6.31 (bs, 1H), |
| | | | | | 6.32 (d, 1H), 7.09 (d, |
| | | | | | 2H), 7.19 (d, 2H), 7.23 |
| | | | | | (dd, 1H), 7.26-7.31 (m, |
| | | | | | 2H), 7.42 (ddd, 1H), 8.87 |
| | | | | | (d, 1H). |
| 18.4 | 2- | N-(2-chloro-4- | 68% | 470 | (DMSOd6) 1.85-1.95 (m, |
| | chloro- | fluorobenzoyl)- | | [M + H]⁺ | 2H), 2.50 (t partially hidden |
| | 4-fluoro | 4-{3-[6- | | | by DMSOd5, 2H), 2.57 (t, |
| | | (methylamino) | | | 2H), 2.73 (d, 3H), 2.91 (dd, |
| | | pyridin-2- | | | 1H), 3.14 (dd, 1H), 4.57 |
| | | yl]propyl}-L- | | | (ddd, 1H), 6.22 (d, 1H), |
| | | phenylalanine | | | 6.29 (q, 1H), 6.31 (d, 1H), |
| | | | | | 7.12 (d, 2H), 7.20 (d, 2H), |
| | | | | | 7.23-7.30 (m, 3H), 7.44 |
| | | | | | (dd, 1H), 8.72 (d, 1H) |
| 18.5 | 2- | N-(2-chloro-6- | 69% | 484 | (DMSOd6) 1.84-1.95 (m, |
| | chloro- | fluoro-3- | | [M + H]⁺ | 2H), 2.26 (s, 3H), 2.51 (t |
| | 6- | methylbenzoyl)- | | | partially hidden by |
| | fluoro | 4-{3-[6- | | | DMSOd5, 2H), 2.56 (t, |
| | 3- | (methylamino) | | | 2H), 2.74 (d, 3H), 2.90 |
| | methyl | pyridin-2- | | | (dd, 1H), 3.10 (dd, 1H), |
| | | yl]propyl}-L- | | | 4.55 (ddd, 1H), 6.21 (d, |
| | | phenylalanine | | | 1H), 6.29 (q, 1H), 6.32 |
| | | | | | (d, 1H), 7.09 (d, 2H), |
| | | | | | 7.14 (dd, 1H), 7.18 (d, |
| | | | | | 2H), 7.27 (dd, 1H), 7.38 |
| | | | | | (dd, 1H), 8.80 (d, 1H) |

The methyl 4-{3-[6-(methylamino)pyridin-2-yl]propyl}-L-phenylalaninate used as the starting material was prepared as follows:

A solution of tert-butyl [6-bromopyridin-2-yl]methylcarbamate (800 mg, 2.79 mmol) described in WO 200411333, 2-allyl-4,4,5,5-tetramethyl-1,3,2-dioxaborolane (0.94 ml, 3.07 mmol), Pd(PPh₃)₄ (312 mg, 0.28 mmol) and cesium fluoride (1.26 g, 8.36 mmol) in THF (3 ml) was degassed, then heated to 80° C. for 1 hour. The reaction mixture was cooled down, diluted in dichloromethane (100 ml), washed with water and dried with MgSO₄. After concentration, a purification by silica gel flash chromatography (50-50 heptane/dichloromethane) afforded tert-butyl (6-allylpyridin-2-yl)methylcarbamate as a yellow oil (540 mg, 79%); ¹H NMR Spectrum (DMSO-d6) 1.45 (s, 9H), 3.27 (s, 3H), 3.47 (d, 2H), 5.09 (ddd, 1H), 5.13 (ddd, 1H), 5.99-6.10 (m, 1H), 6.98 (d, 1H), 7.42 (d, 1H), 7.67 (t, 1H).

A solution of tert-butyl (6-allylpyridin-2-yl)methylcarbamate (540 mg, 2.2 mmol), methyl 4-bromo-N-(tert-butoxycarbonyl)-L-phenylalaninate (795 mg, 2.22 mmol) which is commercially available, tri-O-tolylphosphine (134 mg, 0.44 mmol), Palladium(II) acetate (49 mg, 0.22 mmol) and DIPEA (386 mg, 2.22 mmol) in acetonitrile (2 ml) was degassed, then sealed and heated to 150° C. for 20 minutes in microwave. After concentration, a purification by silica gel flash chromatography (pentane then 0 to 20% ethyl acetate in dichloromethane) afforded methyl N-(tert-butoxycarbonyl)-4-(3-{6-[(tert-butoxycarbonyl)(methyl)amino]pyridin-2-yl}prop-1-en-1-yl)-L-phenylalaninate as a cis-trans mixture (800 mg, 69%); Mass Spectrum [M+H]⁺=526.

A mixture of methyl N-(tert-butoxycarbonyl)-4-(3-{6-[(tert-butoxycarbonyl)(methyl)amino]pyridin-2-yl}prop-1-en-1-yl)-L-phenylalaninate (800 mg, 1.52 mmol) and Pd/C 10% (200 mg) in ethanol (30 ml) was hydrogenated under 1 bar of H₂ overnight, filtered through celite and purified by C18 reverse phase chromatography (acidic conditions) to afford methyl N-(tert-butoxycarbonyl)-4-(3-{6-[(tert-butoxycarbonyl)(methyl)amino]pyridin-2-yl}propyl)-L-phenylalaninate as a colorless gum (540 mg, 67%); Mass Spectrum [M+H]⁺=528; ¹H NMR Spectrum (DMSO-d6) 1.31 (s, 9H), 1.45 (s, 9H), 1.89-1.99 (m, 2H), 2.58 (t, 2H), 2.68 (t, 2H), 2.80 (dd, 1H), 2.94 (dd, 1H), 3.27 (s, 3H), 3.60 (s, 3H), 4.10-4.18 (m, 1H), 6.97 (d, 1H), 7.10-7.16 (m, 4H), 7.27 (d, 1H), 7.39 (d, 1H), 7.64 (dd, 1H). A minor rotamer can also be seen on the spectrum.

To a solution of methyl N-(tert-butoxycarbonyl)-4-(3-{6-[(tert-butoxycarbonyl)(methyl)amino]pyridin-2-yl}propyl)-L-phenylalaninate (540 mg, 1.02 mmol) in MeOH (1 ml) was added a solution of HCl 4M in dioxane (1 ml). The reaction mixture was stirred overnight, then concentrated to dryness to give methyl 4-{3-[6-(methylamino)pyridin-2-yl]propyl}-L-phenylalaninate dihydrochloride (300 mg, 73%), which was used without further purification in the next step; Mass Spectrum [M+H]⁺=328; ¹H NMR Spectrum (DMSO-d6) 1.90-2.01 (m, 2H), 2.59-2.66 (m, 2H), 2.73-2.82 (m, 2H), 2.96 (bs, 3H), 3.00-3.13 (m, 2H), 3.68 (s, 3H), 4.26-4.33 (m, 1H), 6.71 (bs, 1H), 6.88 (bs, 1H), 7.14 (d, 2H), 7.20 (d, 2H), 7.84 (bs, 1H), 8.44 (bs, 3H).

### Example 19

### N-(2,6-dichlorobenzoyl)-3-fluoro-O-{2-[6-(methylamino)pyridin-2-yl]ethyl}-L-tyrosine

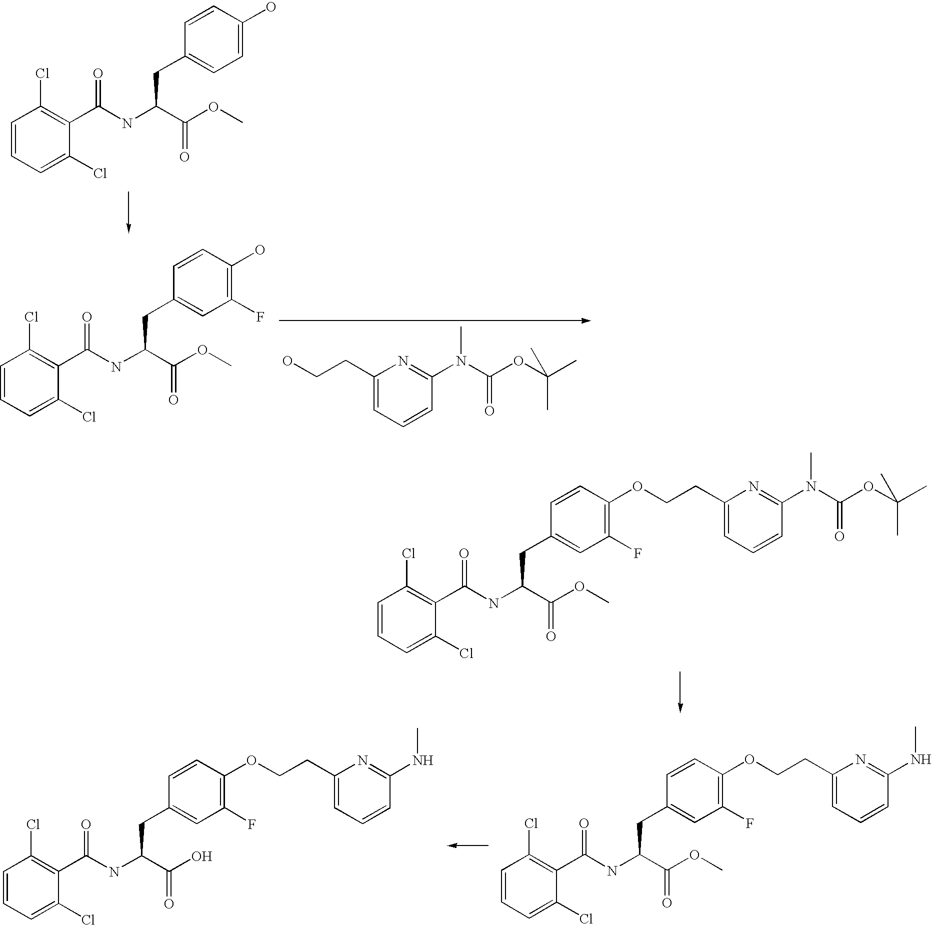

To a solution of methyl N-(2,6-dichlorobenzoyl)-3-fluoro-O-{2-[6-(methylamino)pyridin-2-yl]ethyl}-L-tyrosinate hydrochloride (200 mg, 0.36 mmol) in acetonitrile (3 ml) was added an aqueous solution of NaOH 2N (1 ml, 2 mmol). The reaction was stirred at room temperature 2 hours, filtered, acidified with TFA and purified by C18 reverse phase chromatography (acidic conditions) to afford the title compound as a pale yellow solid (92 mg, 51%); Mass Spectrum [M+H]⁺=506; ¹H NMR Spectrum (DMSO-d6) 2.84 (dd, 1H) 2.91 (bs, 3H), 3.08 (dd, 1H), 3.16 (bs, 2H), 4.33 (t, 2H), 4.63 (ddd, 1H), 7.00 (s, 1H), 7.05 (dd, 1H), 7.09-7.16 (m, 3H), 7.20 (s, 1H), 7.40 (dd, 1H), 7.42-7.46 (m, 2H), 6.71 (bs, 2H), 7.71 (bs, 1H), 9.05 (d, 1H).

The methyl N-(2,6-dichlorobenzoyl)-3-fluoro-O-{2-[6-(methylamino)pyridin-2-yl]ethyl}-L-tyrosinate hydrochloride used as the starting material was prepared as follows:

A solution of methyl N-(2,6-dichlorobenzoyl)-L-tyrosinate (600 mg, 1.63 mmol) and 1-chloromethyl-4-fluoro-1,4-diazoniabicyclo[2.2.2]octane (1.15 g, 3.26 mmol) in acetonitrile (3 ml) was heated at 70° C. for 3 hours. The reaction mixture was cooled, filtered and purified by C18 reverse phase chromatography (acidic conditions) to afford methyl N-(2,6-dichlorobenzoyl)-3-fluoro-L-tyrosinate (250 mg, 40%); Mass Spectrum [M+H]⁺=386; ¹H NMR Spectrum (DMSO-d6) 2.83 (dd, 1H), 3.03 (dd, 1H), 3.65 (s, 3H), 4.66 (ddd, 1H), 6.63 (dd, 1H), 6.88 (dd, 1H), 7.06 (dd, 1H), 7.41 (dd, 1H), 7.44-7.48 (m, 2H), 9.16 (d, 1H), 9.63 (bs, 1H).

To a solution of methyl N-(2,6-dichlorobenzoyl)-3-fluoro-L-tyrosinate (215 mg, 0.58 mmol) in dichloromethane (5 ml) at room temperature were added triphenylphosphine (307 mg, 1.17 mmol) and tert-Butyl [6-(2-hydroxyethyl)pyridin-2-yl]methylcarbamate (176 mg, 0.70 mmol) (Journal of Medicinal Chemistry (2000), 43(1), 22-26). DTAD was then added (269 mg, 1.17 mmol). The reaction mixture was allowed to stir at room temperature overnight, and was then concentrated and purified by C18 reverse phase chromatography (basic conditions) to afford methyl O-(2-{6-[(tert-butoxycarbonyl)(methyl)amino]pyridin-2-yl}ethyl)-N-(2,6-dichlorobenzoyl)-3-fluoro-L-tyrosinate as a white solid (250 mg, 69%); Mass Spectrum [M+H]⁺=620; ¹H NMR Spectrum (DMSO-d6) 1.45 (s, 9H), 2.86 (dd, 1H), 3.08 (dd, 1H), 3.14 (t, 2H), 3.27 (s, 3H), 3.66 (s, 3H), 4.38 (s, 2H), 4.70 (ddd, 1H), 7.02 (dd, 1H), 7.08 (d, 1H), 7.11-7.15 (m, 2H), 7.40 (dd, 1H), 7.43-7.48 (m, 3H), 7.69 (dd, 1H), 9.16 (d, 1H).

To a solution of methyl O-(2-{6-[(tert-butoxycarbonyl)(methyl)amino]pyridin-2-yl}ethyl)-N-(2,6-dichlorobenzoyl)-3-fluoro-L-tyrosinate (200 mg, 0.32 mmol) in MeOH (0.5 ml) was added a solution of HCl 4M in dioxane (2 ml). The reaction mixture was stirred at room temperature overnight, then concentrated to dryness to give methyl N-(2,6-dichlorobenzoyl)-3-fluoro-O-{2-[6-(methylamino)pyridin-2-yl]ethyl}-L-tyrosinate hydrochloride (166 mg, 93%), which was used without further purification in the next step; Mass Spectrum [M+H]⁺=520.

### Example 20.1

### N-(2,6-dichlorobenzoyl)-3-methyl-O-{2-[6-(methylamino)pyridin-2-yl]ethyl}-L-tyrosine and

### Example 20.2

### N-(2,6-dichlorobenzoyl)-3,5-dimethyl-O-{2-[6-(methylamino)pyridin-2-yl]ethyl}-L-tyrosine

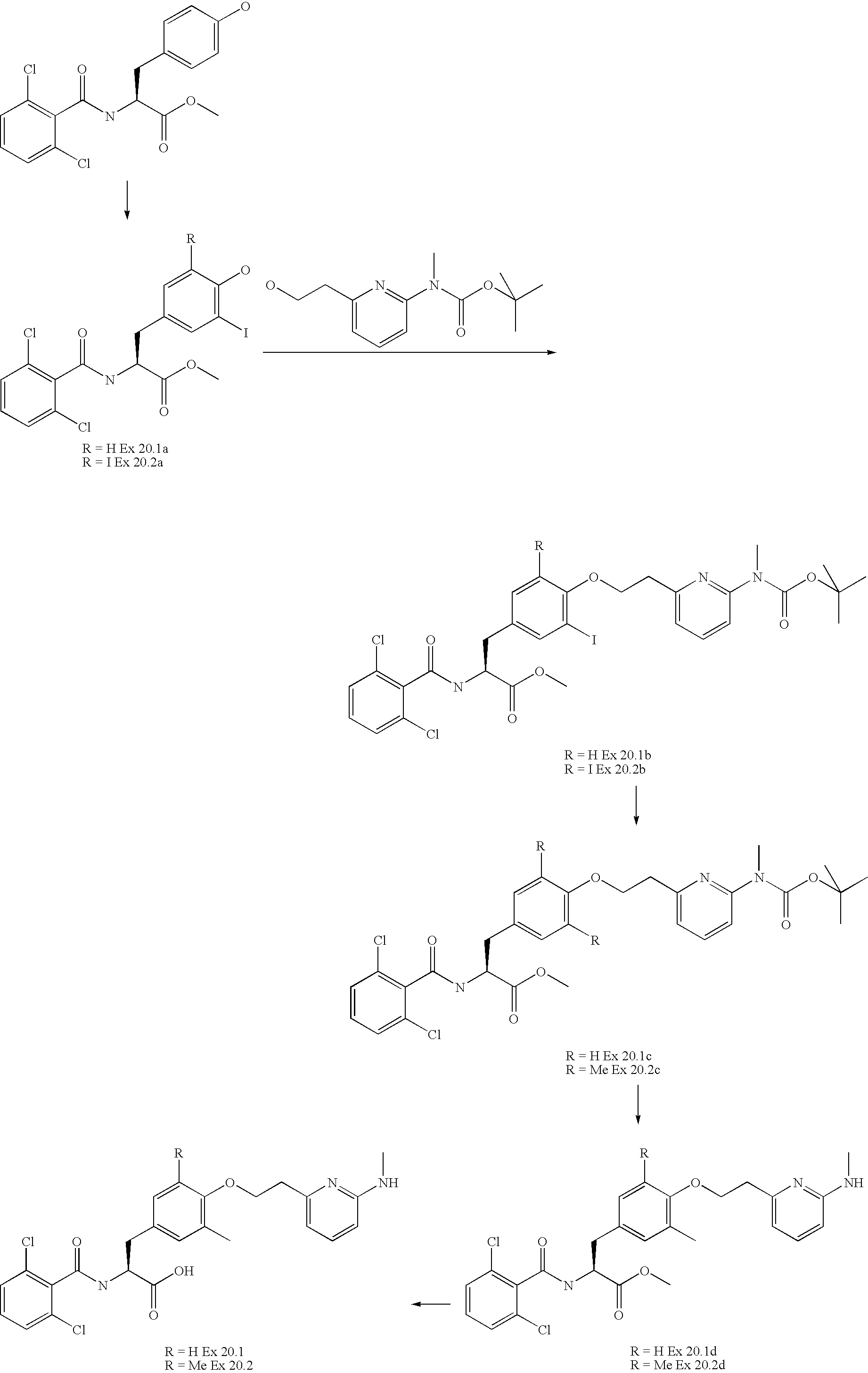

To a solution of methyl N-(2,6-dichlorobenzoyl)-3-methyl-O-{2-[6-(methylamino)pyridin-2-yl]ethyl}-L-tyrosinate hydrochloride (220 mg, 0.40 mmol) in acetonitrile (1 ml) was added an aqueous solution of NaOH 2N (1 ml, 2 mmol). The reaction was stirred at room temperature 1 hour, filtered, acidified with TFA and purified by C18 reverse phase chromatography (acidic conditions) to afford Example 20.1 (84 mg, 1042%); Mass Spectrum [M+H]⁺=502; ¹H NMR Spectrum (DMSO-d6) 2.02 (s, 3H), 2.81 (dd, 1H) 2.93 (s, 3H), 3.01 (dd, 1H), 3.20 (bs, 2H), 4.25 (t, 2H), 4.58 (ddd, 1H), 6.77 (bs, 2H), 6.86 (d, 1H), 7.03-7.09 (m, 3H), 7.40 (dd, 1H), 7.42-7.46 (m, 2H), 7.81 (bs, 1H), 9.04 (d, 1H).

The same procedure was used for Example 20.2 with 33% yield; Mass Spectrum [M+H]⁺=516; ¹H NMR Spectrum (DMSO-d6) 2.07 (s, 6H), 2.74 (d, 3H), 2.84 (dd, 1H), 2.94 (t, 2H), 3.01 (dd, 1H), 4.01, (t, 2H), 4.38 (bs, 1H), 6.26 (d, 1H), 6.33 (q, 1H), 6.45 (q, 1H), 6.88 (s, 2H), 7.30 (dd, 1H), 7.38 (dd, 1H), 7.41-7.46 (m, 2H), 8.46 (bs, 1H).

The methyl N-(2,6-dichlorobenzoyl)-3-methyl-O-{2-[6-(methylamino)pyridin-2-yl]ethyl}-L-tyrosinate hydrochloride and methyl N-(2,6-dichlorobenzoyl)-3,5-dimethyl-O-{2-[6-(methylamino)pyridin-2-yl]ethyl}-L-tyrosinate used as the starting materials were prepared as follows:

To a solution of methyl N-(2,6-dichlorobenzoyl)-L-tyrosinate (1.0 g, 2.72 mmol) in DMF (10 ml) was added at 0° C. benzyltrimethylammonium dichloroiodate (1.04 g, 2.99 mmol) over 10 minutes. The reaction mixture was then stirred at room temperature for 3 hours, filtered and purified by C18 reverse phase chromatography (acidic conditions) to afford methyl N-(2,6-dichlorobenzoyl)-3-iodo-L-tyrosinate (Example 20.1a) (400 mg, 30%) and methyl N-(2,6-dichlorobenzoyl)-3,5-diiodo-L-tyrosinate (Example 20.2a)(300 mg, 17%) as solids;

### Example 20.1a

Mass Spectrum [M+H]⁺=494; ¹H NMR Spectrum (DMSO-d6) 2.81 (dd, 1H), 3.01 (dd, 1H), 3.66 (s, 3H), 4.63 (ddd, 1H), 6.78 (d, 1H), 7.10 (dd, 1H), 7.41 (dd, 1H), 7.44-7.49 (m, 2H), 7.56 (d, 1H), 9.16 (d, 1H) 10.12 (bs, 1H).

### Example 20.2a

Mass Spectrum [M+H]⁺=620; ¹H NMR Spectrum (DMSO-d6) 2.80 (dd, 1H), 3.03 (dd, 1H), 3.67 (s, 3H), 4.65 (ddd, 1H), 7.41 (dd, 1H), 7.45-7.50 (m, 2H), 7.64 (s, 2H), 9.16 (s, 1H), 9.36 (bs, 1H).

To a solution of methyl N-(2,6-dichlorobenzoyl)-3-iodo-L-tyrosinate (Example 20.1a) (440 mg, 0.89 mmol) in dichloromethane (3 ml) at room temperature were added triphenylphosphine (466 mg, 1.78 mmol) and tert-Butyl [6-(2-hydroxyethyl)pyridin-2-yl]methylcarbamate (338 mg, 1.34 mmol) (Journal of Medicinal Chemistry (2000), 43(1), 22-26). DTAD was then added (409 mg, 1.78 mmol). The reaction mixture was allowed to stir at room temperature 48 hours, and was then concentrated and purified by C18 reverse phase chromatography (basic conditions) to afford methyl O-(2-{6-[(tert-butoxycarbonyl)(methyl)amino]pyridin-2-yl}ethyl)-N-(2,6-dichlorobenzoyl)-3-iodo-L-tyrosinate (Example 20.1b) (400 mg, 62%); Mass Spectrum [M+H]⁺=728; ¹H NMR Spectrum (DMSO-d6) 1.45 (s, 9H), 2.85 (dd, 1H), 3.06 (dd, 1H), 3.14 (t, 2H), 3.27 (s, 3H), 3.66 (s, 3H), 4.35 (t, 2H), 4.66 (ddd, 1H), 6.97 (d, 1H), 7.13 (d, 1H), 7.26 (dd, 1H), 7.40 (dd, 1H), 7.43-7.48 (m, 3H), 7.65 (d, 1H), 7.68 (t, 1H), 9.17 (d, 1H).

The same procedure was used for the preparation of methyl O-(2-{6-[(tert-butoxycarbonyl)(methyl)amino]pyridin-2-yl}ethyl)-N-(2,6-dichlorobenzoyl)-3,5-diiodo-L-tyrosinate (Example 20.2b) with 66% yield; Mass Spectrum [M+H]⁺=854; ¹H NMR Spectrum (DMSO-d₆) 1.45 (s, 9H), 2.82 (dd, 1H) 3.12 (dd, 1H), 3.25 (t, 2H), 3.30 (s, 3H), 3.68 (s, 3H), 4.22 (t, 2H), 4.74 (ddd, 1H), 7.15 (d, 1H), 7.41 (dd, 1H), 7.43-7.48 (m, 3H), 7.62 (dd, 1H), 7.70 (dd, 1H), 7.76 (s, 2H), 9.14 (d, 1H).

A mixture of methyl O-(2-{6-[(tert-butoxycarbonyl)(methyl)amino]pyridin-2-yl}ethyl)-N-(2,6-dichlorobenzoyl)-3-iodo-L-tyrosinate (Example 20.1b) (344 mg, 0.47 mmol), trimethylboroxine (0.2 ml, 1.42 mmol), PdCl₂(dppf). CH₂Cl₂ (38 mg, 0.047 mmol) and Cesium carbonate (463 mg, 1.42 mmol) in 1,4 dioxane (2 ml) was degassed and heated at 85° C. for 2 hours. The reaction mixture was cooled, filtered through celite, concentrated and purified by C18 reverse phase chromatography (basic conditions) to afford methyl O-(2-{6-[(tert-butoxycarbonyl)(methyl)amino]pyridin-2-yl}ethyl)-N-(2,6-dichlorobenzoyl)-3-methyl-L-tyrosinate (Example 20.1c) as a white solid (230 mg, 79%); Mass Spectrum [M+H]⁺=616; ¹H NMR Spectrum (DMSOd6) 1.45 (s, 9H), 1.98 (s, 3H), 2.84 (dd, 1H), 3.00 (dd, 1H), 3.13 (t, 2H), 3.26 (s, 3H), 3.64 (s, 3H), 4.29 (t, 2H), 4.62 (ddd, 1H), 6.87 (d, 1H), 6.99-7.06 (m, 2H), 7.08 (d, 1H), 7.37-7.48 (m, 4H), 7.68 (t, 1H), 9.17 (d, 1H).

The same procedure was used for the preparation of methyl O-(2-{6-[(tert-butoxycarbonyl)(methyl)amino]pyridin-2-yl}ethyl)-N-(2,6-dichlorobenzoyl)-3,5-dimethyl-L-tyrosinate (Example 20.2c) as a brown gum with 48% yield; Mass Spectrum [M−H]-=628; ¹H NMR Spectrum (DMSOd6) 1.45 (s, 9H), 2.04 (s, 6H), 2.80 (dd, 1H), 2.99 (dd, 1H), 3.12 (t, 2H), 3.27 (s, 3H), 4.06 (t, 2H), 4.64 (ddd, 1H), 6.89 (s, 2H) 7.13 (d, 1H), 7.40 (dd, 1H), 7.42-7.47 (m, 3H), 7.69 (t, 1H), 9.15 (s, 1H).

To a solution of methyl O-(2-{6-[(tert-butoxycarbonyl)(methyl)amino]pyridin-2-yl}ethyl)-N-(2,6-dichlorobenzoyl)-3-methyl-L-tyrosinate (Example 20.1c) (250 mg, 0.40 mmol) in MeOH (0.5 ml) was added a solution of HCl 4M in dioxane (2 ml). The reaction mixture was stirred at room temperature overnight, then concentrated to dryness to give methyl N-(2,6-dichlorobenzoyl)-3-methyl-O-{2-[6-(methylamino)pyridin-2-yl]ethyl}-L-tyrosinate hydrochloride (Example 20. Id) (166 mg, 74%), which was used without further purification in the next step; Mass Spectrum [M+H]⁺=516.

The same procedure was used for the preparation of methyl N-(2,6-dichlorobenzoyl)-3,5-dimethyl-O-{2-[6-(methylamino)pyridin-2-yl]ethyl}-L-tyrosinate hydrochloride (Example 20.2d) with 93% yield; Mass Spectrum [M+H]⁺=530.

### Example 21

The compound of Example 21.1 described below and the compounds shown in Table 6 were prepared using the following reaction scheme:

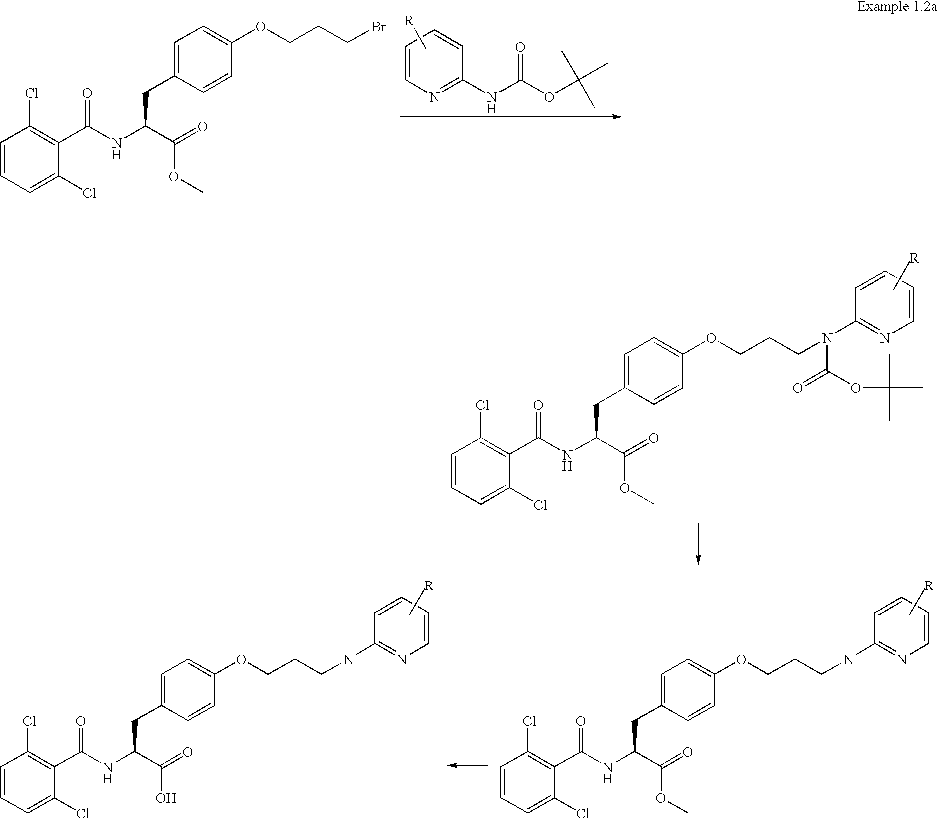

### Example 21.1

### N-(2,6-dichlorobenzoyl)-O-{3-[(5-methylpyridin-2-yl)amino]propyl}-L-tyrosine

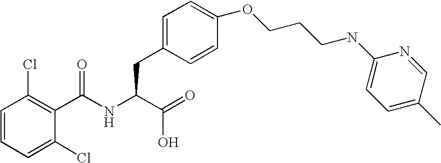

LiOH (50 mg) was added to a solution of methyl N-(2,6-dichlorobenzoyl)-O-{3-[(5-methylpyridin-2-yl)amino]propyl}-L-tyrosinate (150 mg, 0.29 mmol) in a mixture DMA/H₂O (1.5 ml/1.0 ml). After 3 hours at room temperature and 1 hour at 40° C., the crude reaction mixture was purified by C18 reverse phase chromatography (basic conditions) to afford the title compound as a solid (70 mg, 48%); Mass Spectrum [M+H]⁺=502; ¹H NMR Spectrum (DMSO-d₆) 1.88-1.99 (m, 2H), 2.08 (s, 3H), 2.89 (dde 1H), 3.05 (dd, 1H), 3.29-3.36 (m, 2H), 3.99 (t, 2H), 4.45-4.54 (m, 1H), 6.32 (t, 1H), 6.39 (d, 1H), 6.81 (d, 2H), 7.14-7.22 (3H), 7.38 (dd, 1H), 7.40-7.45 (m, 2H), 7.79 (bs, 1H), 8.67 (d, 1H).

### Examples 21.2 to 21.6

The procedure described above for Example 21.1 was repeated using the appropriate methyl ester precursor. Thus were obtained the compounds shown in Table 6:

TABLE 6

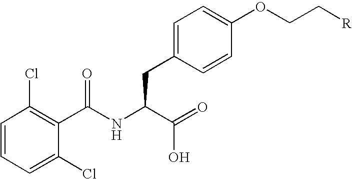

| | | | | Mass | ¹H NMR Data |
|---|---|---|---|---|---|
| Example | R | Name | Yield | Ion | (500 MHz) |
| 21.2 | 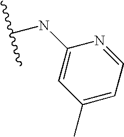 | N-(2,6- dichlorobenzoyl)- O-{3-[(4- methylpyridin- 2- yl)amino]propyl]- L-tyrosine | 51% | 502 [M + H]⁺ | (DMSOd6) 1.88- 1.98 (m, 2H), 2.12 (s, 3H), 2.91 (dd, 1H), 3.05 (dd, 1H), 3.31-3.37 (m, 2H), 3.99 (t, 2H), 4.39- 4.48 (m, 1H), 6.26 (bs, 1H), 6.30 (d, |
| # 1H), 6.42 (t, 1H), 6.80 (d, 2H), 7.17 (d, 2H), 7.38 (dd, 1H), 7.41-7.45 (m, 2H), 7.81 (d, 1H), 8.57 (bs, 1H) | | | | | |
| 21.3 | 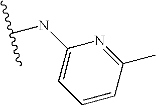 | N-(2,6- dichlorobenzoyl)- O-{3-[(6- methylpyridin- 2- yl)amino]propyl}- L-tyrosine | 43% | 502 [M + H]⁺ | (DMSOd6) 1.91- 1.99 (m, 2H), 2.23 (s, 3H), 2.90 (dd, 1H), 3.04 (dd, 1H), 3.31-3.35 (m, 2H), 4.00 (t, 2H), 4.40- 4.52 (m, 1H), 6.24 (d, 1H), 6.31 (d, |
| # 1H), 6.41 (t, 2H), 6.80 (d, 2H), 7.17 (d, 2H), 7.23 (dd, 1H), 7.38 (dd, 1H), 7.41-7.46 (m, 2H), 8.63 (m, 1H) | | | | | |
| 21.4 | 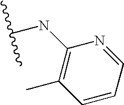 | N-(2,6- dichlorobenzoyl)- O-{3-[(3- methylpyridin- 2- yl)amino]propyl}- L-tyrosine | 41% | 502 [M + H]⁺ | (DMSOd6) 1.96- 2.02 (m, 2H), 2.03 (s, 3H), 2.90 (dd, 1H), 3.05 (dd, 1H), 3.43-3.51 (m, 2H), 4.00 (t, 2H), 4.42- 4.51 (m, 1H), 5.91 (t, 1H), 6.43 (dd, |
| # 1H), 6.80 (d, 2H), 7.18 (d, 2H) 7.19 (d, 1H), 7.38 (dd, 1H), 7.41-7.45 (m, 2H), 7.86 (dd, 1H), 8.60 (bs, 1H) | | | | | |
| 21.5 | 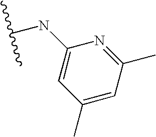 | N-(2,6- dichlorobenzoyl)- O-{3- [(4,6- dimethylpyridin- 2- yl)amino]propyl}- L-tyrosine | 63% | 516 [M + H]⁺ | (DMSOd6) 1.89- 1.99 (m, 2H), 2.08 (s, 3H), 2.19 (s, 3H), 2.87 (dd, 1H), 3.05 (dd, 1H), 3.29-3.35 (m, 2H), 4.00 (t, 2H), 4.58 (ddd, 1H), |
| #6.06 (s, 1H), 6.17 (s, 1H), 6.31 (t, 1H), 6.83 (d, 2H), 7.19 (d, 2H), 7.39 (dd, 1H), 7.41-7.45 (m, 2H), 8.96 (d, 1H) | | | | | |
| 21.6 | 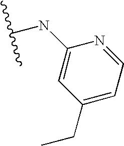 | N-(2,6- dichlorobenzoyl)- O-{3-[(4- ethylpyridin- 2- yl)amino]propyl}- L-tyrosine | 63% | 516 [M + H]⁺ | (DMSOd6) 1.12 (t, 3H), 1.89-1.98 (m, 2H), 2.42 (q, 2H), 2.87 (dd, 1H), 3.05 (dd, 1H), 3.32-3.38 (m, 2H), 4.00 (t, 2H), 4.52-4.61 (m, 1H), 6.28 (s, 1H), |
| #6.34 (d, 1H), 6.43 (t, 1H), 6.83 (d, 2H), 7.18 (d, 2H), 7.38 (dd, 1H), 7.41-7.46 (m, 2H), 7.84 (d, 1H), 8.89 (bs, 1H) | | | | | |

The starting materials used in Examples 21.1 to 21.6 were prepared as follows:

Sodium hydride (60% in oil, 39 mg, 0.98 mmol) was added to a solution of tert-butyl-5-methylpyridin-2-ylcarbamate (204 mg, 0.98 mmol) which is commercially available in DMA (3 ml) at 0° C. The temperature was raised to room temperature for 15 minutes, then methyl O-(3-bromopropyl)-N-(2,6-dichlorobenzoyl)-L-tyrosinate (Example 1.2a) was added (400 mg, 0.82 mmol) and the reaction mixture stirred at room temperature overnight. Dichloromethane was added and the organic layer was washed with a saturated solution of ammonium chloride and evaporated. The residue was dissolved in dichloromethane and purified by silica gel flash chromatography (0 to 40% Ethyl acetate in petroleum ether) to give methyl O-{3-[(tert-butoxycarbonyl)(5-methylpyridin-2-yl)amino]propyl}-N-(2,6-dichlorobenzoyl)-L-tyrosinate example 21.1a as a colorless oil (411 mg, 82%).

The procedure described above for the preparation of methyl O-{3-[(tert-butoxycarbonyl)(5-methylpyridin-2-yl)amino]propyl}-N-(2,6-dichlorobenzoyl)-L-tyrosinate example 21.1a was repeated using the appropriate BOC-aminopyridine precursor. Thus were obtained the compounds described in Table 6.1:

TABLE 6.1

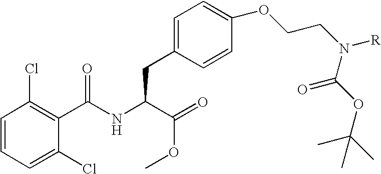

| | | | | Mass | ¹H NMR Data |
|---|---|---|---|---|---|
| Example | R | Name | Yield | Ion | (500 MHz) |
| 21.1a | 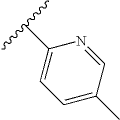 | methyl O-{3- [(tert- butoxycarbonyl) (5-methylpyridin- 2- yl)amino]propyl}- N-(2,6- dichlorobenzoyl)- L-tyrosinate | 82% | 616 [M + H]⁺ | (DMSOd6) 1.41 (s, 9H), 1.90-1.98 (m, 2H), 2.24 (s, 3H), 2.87 (dd, 1H), 3.03 (dd, 1H), 3.64 (s, 3H), 3.93 (t, 2H), |
| #3.96 (t, 2H), 4.65 (ddd, 1H), 6.67 (d, 2H), 7.15 (d, 2H), 7.38-7.48 (m, 4H), 7.56 (dd, 1H), 8.19 (bs, 1H), 9.18 (d, 1H) | | | | | |
| 21.2a | 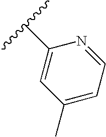 | methyl O-{3- [(tert- butoxycarbonyl) (4-methylpyridin 2- yl)amino]propyl}- N-(2,6- dichlorobenzoyl)- L-tyrosinate | 63% | 616 [M + H]⁺ | (DMSOd6) 1.43 (s, 9H), 1.92-2.00 (m, 2H), 2.29 (s, 3H), 2.88 (dd, 1H), 3.03 (dd, 1H), 3.64 (s, 3H), 3.94 (t, 2H), |
| #3.99 (t, 2H), 4.65 (ddd, 1H), 6.77 (d, 2H), 6.97 (d, 1H), 7.16 (d, 2H), 7.38 (s, 1H), 7.41 (dd, 1H), 7.43-7.48 (m, 2H), 8.22 (d, 1H), 9.18 (d, 1H) | | | | | |
| 21.3a | 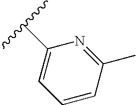 | methyl O-{3- [(tert- butoxycarbonyl) 6-methylpyridin- 2- yl)amino]propyl}- N-(2,6- dichlorobenzoyl)- L-tyrosinate | 42% | 616 [M + H]⁺ | (DMSOd6) 1.42 (s, 9H), 1.93-2.02 (m, 2H), 2.39 (s, 3H), 2.88 (dd, 1H), 3.03 (dd, 1H), 3.65 (s, 3H), 3.95 (t, 2H), |
| #3.98 (t, 2H), 4.65 (ddd, 1H), 6.77 (d, 2H), 6.98 (d, 1H), 7.16 (d, 2H), 7.32 (d, 1H), 7.41 (dd, 1H), 7.43-7.47 (m, 2H), 7.62 (dd, 1H), 9.18 (d, 1H) | | | | | |
| 21.4a | 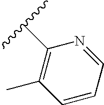 | methyl O-{3- [(tert- butoxycarbonyl) 3-methylpyridin- 2- yl)amino]propyl}- N-(2,6- dichlorobenzoyl)- L-tyrosinate | 84% | 616 [M + H]⁺ | (DMSOd6) 1.34 (bs, 9H), 1.90 (bs, 3H), 2.19 (bs, 3H), 2.87 (dd, 1H), 3.04 (dd, 1H), 3.64 (s, 3H), 3.75 (bs, 2H), 3.94 |
| #(bs, 2H), 4.65 (ddd, 1H), 6.77 (d, 2H), 7.16 (d, 2H), 7.24 (dd, 1H), 7.40 (dd, 1H), 7.43-7.47 (m, 2H), 7.71 (d, 1H), 8.29 (dd, 1H), 9.19 (d, 1H) | | | | | |
| 21.5a | 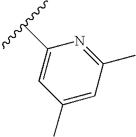 | methyl O-{3- [(tert- butoxycarbonyl) (4,6- dimethylpyridin- 2- yl)amino]propyl}- N-(2,6- dichlorobenzoyl)- L-tyrosinate | 53% | 630 [M + H]⁺ | (DMSOd6) 1.44 (s, 9H), 1.94-2.03 (m, 2H), 2.25 (s, 3H), 2.36 (s, 3H), 2.90 (dd, 1H), 3.05 (dd, 1H), 3.66 (s, 3H), |
| # 3.93-4.01 (m, 4H), 4.67 (ddd, 1H), 6.78 (d, 2H), 6.84 (s, 1H), 7.17 (s, 1H), 7.18 (d, 2H), 7.43 (dd, 1H), 7.45-7.49 (m, 2H), 9.19 (d, 1H) | | | | | |
| 21.6a | 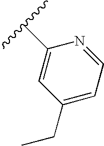 | methyl O-{3- [(tert- butoxycarbonyl) (4-ethylpyridin-2- yl)amino]propyl}- N-(2,6- dichlorobenzoyl)- L-tyrosinate | 64% | 630 [M + H]⁺ | (DMSOd6) 1.16 (t, 3H), 1.43 (s, 9H), 1.91-2.02 (m, 2H), 2.59 (q, 2H), 2.88 (dd, 1H), 3.03 (dd, 1H), 3.64 (s, 3H), 3.94 (t, 2H), |
| #3.99 (t, 2H), 4.65 (ddd, 1H), 6.77 (d, 2H), 7.00 (dd, 1H), 7.15 (d, 2H), 7.39 (s, 1H), 7.41 (dd, 1H), 7.43- 7.48 (m, 2H), 8.24 (d, 1H), 9.18 (d, 1H) | | | | | |

TFA (4 ml) was added to a solution of methyl O-{3-[(tert-butoxycarbonyl)(5-methylpyridin-2-yl)amino]propyl}-N-(2,6-dichlorobenzoyl)-L-tyrosinate example 21.1a (400 mg, 0.65 mmol) in dichloromethane (4 ml). The reaction mixture was stirred for 2 hours at room temperature, then solvent was removed by evaporation. The residue was dissolved in water and NaOH 2N was added dropwise until a white suspension appeared. Filtration of the salts, followed by evaporation of solvents afforded methyl N-(2,6-dichlorobenzoyl)-O-{3-[(5-methylpyridin-2-yl)amino]propyl}-L-tyrosinate example 21.1b as a yellow solid (310 mg, 93%), used without further purification in next step.

The procedure described above for the preparation of methyl N-(2,6-dichlorobenzoyl)-O-{3-[(5-methylpyridin-2-yl)amino]propyl}-L-tyrosinate example 21.1b was repeated using the appropriate N-BOC precursor. Thus were obtained the compounds described in Table 6.2:

TABLE 6.2

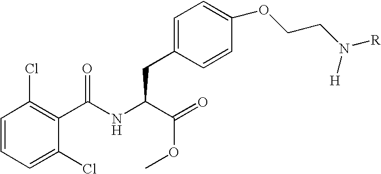

| | | | | Mass | ¹H NMR Data |
|---|---|---|---|---|---|
| Example | R | Name | Yield | Ion | (500 MHz) |
| 21.1b | 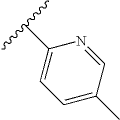 | methyl N- (2,6- dichlorobenzoyl)- O-{3-[(5- methylpyridin- 2-yl)amino]propyl}-L- tyrosinate | 93% | 516 [M + H]⁺ | (DMSOd6) 1.99-2.08 (m, 2H), 2.17 (s, 3H), 2.88 (dd, 1H), 3.05 (dd, 1H), 3.40-3.50 (m, 2H), 3.65 (s, 3H), 4.04 (t, 2H), 4.66 (ddd, 1H), 6.85 (d, 2H), |
| #7.01 (d, 1H), 7.19 (d, 2H), 7.38-7.49 (m, 3H), 7.33 (s, 1H), 7.78 (dd, 1H), 8.67 (bs, 1H), 9.19 (d, 1H) | | | | | |
| 21.2b | 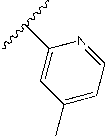 | methyl N- (2,6- dichlorobenzoyl)- O-{3-[(4- methylpyridin- 2-yl)amino]propyl}-L- tyrosinate | 52% | 516 [M + H]⁺ | (DMSOd6) 1.92-1.99 (m, 2H), 2.15 (s, 3H), 2.88 (dd, 1H), 3.04 (dd, 1H), 3.34-3.38 (m, 2H), 3.64 (s, 3H), 4.00 (t, 2H), 4.66 (ddd, 1H), 6.36 (bs, 2H), |
| #6.78 (bs, 1H), 6.84 (d, 2H), 7.18 (d, 2H), 7.40 (dd, 1H), 7.43-7.47 (m, 2H), 7.81 (d, 1H), 9.18 (d, 1H) | | | | | |
| 21.3b | 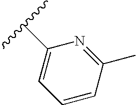 | methyl N- (2,6- dichloro- benzoyl)-O-{3-[(6- methylpyridin-2- 2-yl)amino]propyl}-L- tyrosinate | 85% | 516 [M + H]⁺ | (DMSOd6) 1.98-2.07 (m, 2H), 2.40 (s, 3H), 2.88 (dd, 1H), 3.05 (dd, 1H), 3.44-3.55 (m, 2H), 3.64 (s, 3H), 4.04 (t, 2H), 4.66 (ddd, 1H), 6.62 (bs, 1H), |
| #6.79 (bs, 1H), 6.85 (d, 2H), 7.19 (d, 2H), 7.41 (dd, 1H), 7.48-7.48 (m, 2H), 7.70 (bs, 1H), 9.19 (d, 1H) | | | | | |
| 21.4b | 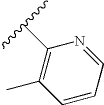 | methyl N- (2,6- dichloro- benzoyl)-O-{3-[(3- methylpyridin-2- yl)amino]propyl}-L- tyrosinate | 80% | 516 [M + H]⁺ | (DMSOd6) 1.98-2.05 (m, 2H), 2.07 (s, 3H), 2.88 (dd, 1H), 3.04 (dd, 1H), 3.45-3.54 (m, 2H), 3.64 (s, 3H), 4.02 (t, 2H), 4.66 (ddd, 1H), 3.60 (bs, 1H), |
| #6.51 (t, 1H), 6.84 (d, 2H), 7.18 (d, 2H), 7.31 (bs, 1H), 7.41 (dd, 1H), 7.43- 7.48 (m, 2H), 7.85 (dd, 1H), 9.19 (d, 1H) | | | | | |
| 21.5b | 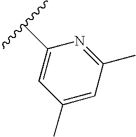 | methyl N- (2,6- dichloro- benzoyl)-O-{3-[(4,6- dimethyl- pyridin-2- yl)amino]propyl}-L- tyrosinate | 100% | 530 [M + H]⁺ | (DMSOd6) 1.96-2.07 (m, 2H), 2.26 (s, 3H), 2.38 (s, 3H), 2.88 (dd, 1H), 3.05 (dd, 1H), 3.46-3.54 (m, 2H), 3.64 (s, 3H), 4.03 (t, 2H), 4.66 |
| #(ddd, 1H), 6.55 (s, 1H), 6.70 (bs, 1H), 6.86 (d, 2H), 7.20 (d, 2H), 7.41 (dd, 1H), 7.43-7.48 (m, 2H), 8.12 (bs, 1H), 9.19 (d, 1H) | | | | | |
| 21.6b | 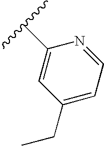 | methyl N- (2,6- dichloro- benzoyl)-O-{3-[(4- ethylpyridin- 2-yl)amino]propyl}-L- tyrosinate | 72% | 530 [M + H]⁺ | (DMSOd6) 1.15 (t, 3H), 1.99-2.08(m, 2H), 2.59 (q, 2H), 2.88 (dd, 1H), 3.03 (dd, 1H), 3.34-3.50 (m, 2H), 3.64 (s, 3H), 4.04 (t, 2H), 4.66 (ddd, |
| #1H), 6.70 (d, 1H), 6.76 (s, 1H), 6.85 (d, 2H), 7.19 (d, 2H), 7.41 (dd, 1H), 7.43- 7.48 (m, 2H), 7.82 (d, 1H), 8.30 (bs, 1H), 9.19 (d, 1H) | | | | | |

### Example 22

### N-(2,6-dichlorobenzoyl)-4-(4-{[6-(methylamino)pyridin-2-yl]methyl}piperidin-1-yl)-L-phenylalanine

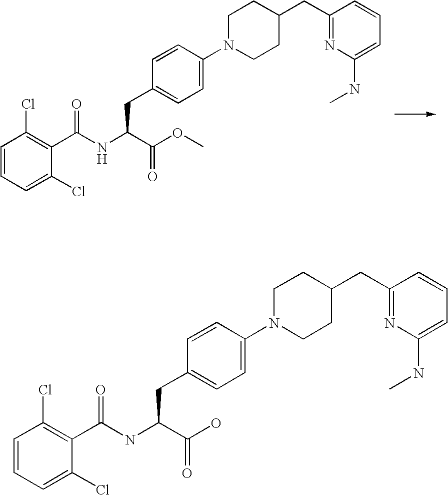

LiOH (5 mg, 0.12 mmol) was added to a solution of methyl N-(2,6-dichlorobenzoyl)-4-(4-{[6-(methylamino)pyridin-2-yl]methyl}piperidin-1-yl)-L-phenylalaninate (130 mg, 0.25 mmol) in DMA/H₂O (1 ml/0.2 ml). After stirring at room temperature for 3 hours, the reaction mixture was purified by C18 reverse phase chromatography (basic conditions) to give the title compound as a white solid (14 mg, 67%); Mass Spectrum [M+H]⁺=541; ¹H NMR Spectrum: (DMSOd6) 1.21-1.35 (m, 2H), 1.61-1.69 (m, 2H), 1.78-1.89 (m, 1H), 2.45 (d, 2H), 2.52-2.61 (m, 2H), 2.73 (d, 3H), 2.82 (dd, 1H), 3.00 (dd, 1H), 3.55-3.63 (m, 2H), 4.52-4.60 (m, 1H), 6.22 (d, 1H), 6.29 (q, 1H), 6.32 (d, 1H), 6.81 (d, 2H), 7.09 (d, 2H), 7.28 (dd, 1H), 7.38 (dd, 1H), 7.41-7.46 (m, 2H), 8.92 (bs, 1H).

The methyl N-(2,6-dichlorobenzoyl)-4-(4-{[6-(methylamino)pyridin-2-yl]methyl}piperidin-1-yl)-L-phenylalaninate used as the starting material was prepared as follows:

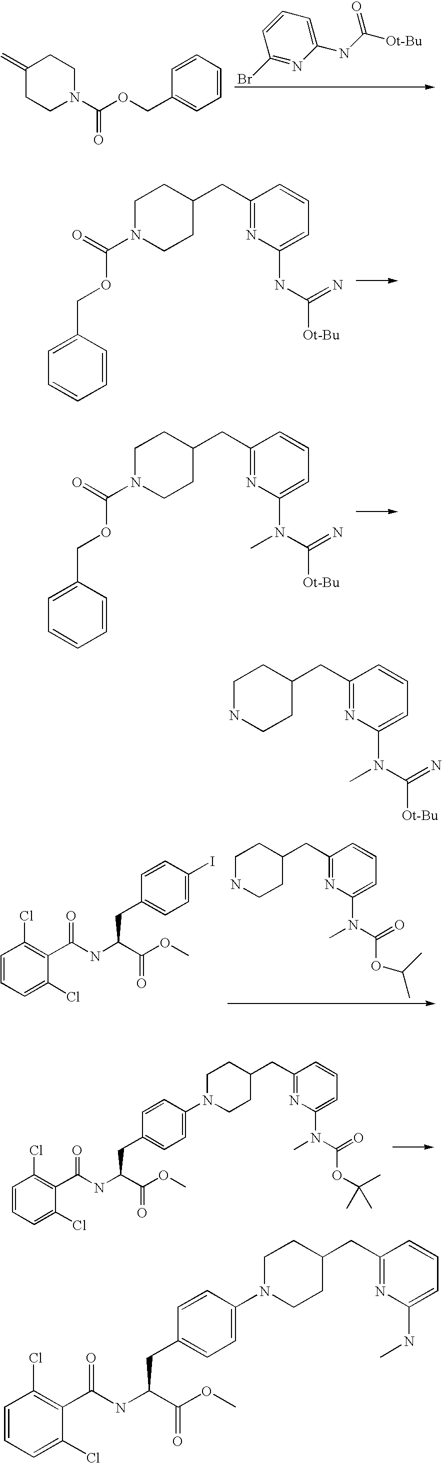

9-BBN (11.6 ml, 5.58 mmol) was added under argon atmosphere to a solution of benzyl 4-methylenepiperidine-1-carboxylate (1.0 g, 5.07 mmol) in THF (15 ml). The mixture was heated at 75° C. for 2 hours. Then tert-butyl (6-bromopyridin-2-yl)carbamate (1.25 g, 4.56 mmol), PdCl₂dppf (122 mg, 0.15 mmol), K₂CO 3 (0.91 g, 6.59 mmol), DMF (10 ml) and H₂O (1 ml) were added. The mixture was heated at 60° C. for 4 hours, then cooled, extracted with ethyl acetate, evaporated and purified by silica gel flash chromatography (10 to 30% ethyl acetate in petroleum ether) to give benzyl 4-({6-[(tert-butoxycarbonyl)amino]pyridin-2-yl}methyl)piperidine-1-carboxylate (1.36 g, 63%); ¹H NMR Spectrum: (CDCl3) 1.19 (bs, 2H), 1.52 (s, 9H), 1.56-1.61 (bs, 2H), 1.83-1.95 (m, 1H), 2.56 (d, 2H), 2.74 (bs, 2H), 4.14 (bs, 2H), 5.11 (s, 2H), 6.75 (d, 1H), 7.15 (bs, 1H), 7.28-7.38 (m, 5H), 7.55 (dd, 1H), 7.73 (d, 1H).

NaH (124 mg, 3.10 mmol) was added at 0° C. to a solution of benzyl 4-({6-[(tert-butoxycarbonyl)amino]pyridin-2-yl}methyl)piperidine-1-carboxylate (1.2 g, 2.82 mmol). After stirring for 15 minutes, methyl iodide (0.35 ml, 5.64 mmol) was added and the mixture was stirred at room temperature for 16 hours. Further amounts of NaH (30 mg) and methyl iodide (0.17 ml) were added to reach completion. After stirring for 4 hours and evaporation to dryness, the residue was extracted with ethyl acetate and purified by silica gel flash chromatography (20 to 30% ethyl acetate in petroleum ether) to give benzyl 4-({6-[(tert-butoxycarbonyl)(methyl)amino]pyridin-2-yl}methyl)piperidine-1-carboxylate (1.1 g, 89%); ¹H NMR Spectrum: (CDCl3) 1.22 (bs, 2H), 1.51 (s, 9H), 1.60-1.71 (m, 2H), 1.90-2.02 (m, 1H), 2.64 (d, 2H), 2.76 (bs, 2H), 3.37 (s, 3H), 4.15 (m, 2H), 5.12 (s, 2H), 6.79 (d, 1H), 7.28-7.38 (m, 5H), 7.46 (d, 1H), 7.51 (dd, 1H).

A solution of benzyl 4-({6-[(tert-butoxycarbonyl)(methyl)amino]pyridin-2-yl}methyl)piperidine-1-carboxylate (1.1 g, 2.5 mmol) in ethanol (100 ml) was hydrogenated under 40 psi for 1.5 hours in the presence of Pd/C 10% (120 mg). The catalyst was filtered off and the resulting mixture evaporated to afford tert-butyl methyl[6-(piperidin-4-ylmethyl)pyridin-2-yl]carbamate (745 mg, 98%) as an oil; ¹H NMR Spectrum: (CDCl3) 1.23-1.37 (m, 2H), 1.61-1.74 (m, 2H), 1.86-1.99 (m, 1H), 2.57-2.71 (m, 4H), 3.08-3.18 (m, 2H), 3.38 (s, 3H), 3.42 (bs, 1H), 6.80 (d, 1H), 7.44 (d, 1H), 7.51 (dd, 1H).

To tert-butyl methyl[6-(piperidin-4-ylmethyl)pyridin-2-yl]carbamate (700 mg, 1.14 mmol) in DMSO (3 ml) were added under argon atmosphere methyl N-(2,6-dichlorobenzoyl)-4-iodo-L-phenylalaninate (300 mg, 0.98 mmol), Cs₂CO₃ (640 mg, 1.96 mmol), CuI (38 mg, 0.20 mmol) and L-proline (45 mg, 0.39 mmol). The mixture was heated at 90° C. for 18 hours, extracted with ethyl acetate and purified by silica gel flash chromatography (20 to 40% ethyl acetate in petroleum ether) to give methyl 4-[4-({6-[(tert-butoxycarbonyl)(methyl)amino]pyridin-2-yl}methyl)piperidin-1-yl]-N-(2,6-dichlorobenzoyl)-L-phenylalaninate (141 mg, 22%); ¹H NMR Spectrum: (CDCl3) 1.36-1.49 (m, 2H), 1.52 (s, 9H), 1.70-1.79 (m, 2H), 1.87-1.98 (m, 1H), 2.58-2.71 (m, 4H), 3.15 (dd, 1H) 3.21 (dd, 1H), 3.38 (s, 3H), 3.57-3.65 (m, 2H), 3.75 (s, 3H), 5.15 (ddd, 1H), 6.24 (d, 1H), 6.79-6.87 (m, 3H), 7.06 (d, 2H), 7.25 (dd, 1H), 7.28-7.33 (m, 2H), 7.45 (d, 1H), 7.52 (dd, 1H).

A solution of methyl 4-[4-({6-[(tert-butoxycarbonyl)(methyl)amino]pyridin-2-yl}methyl)piperidin-1-yl]-N-(2,6-dichlorobenzoyl)-L-phenylalaninate (0.17 g, 0.26 mmol) in TFA (3 ml) was stirred at room temperature for 2 hours. After evaporation, the residue was redissolved in DCM and NH₃/MeOH 7N. After evaporation, the residue was purified by silica gel flash chromatography (3% NH₃/MeOH in dichloromethane) to give methyl N-(2,6-dichlorobenzoyl)-4-(4-{[6-(methylamino)pyridin-2-yl]methyl}piperidin-1-yl)-L-phenylalaninate (118 mg; 84%); ¹H NMR Spectrum: (CDCl3) 1.34-1.46 (m, 2H), 1.70-1.81 (m, 2H), 1.83-1.95 (m, 1H), 2.54 (d, 2H), 2.59-2.67 (m, 2H), 2.87 (d, 3H), 3.5 (dd, 1H), 3.20 (dd, 1H), 3.57-3.63 (m, 1H), 3.75 (s, 3H), 4.64 (bs, 1H), 5.14 (ddd, 1H), 6.23 (d, 1H), 6.25 (d, 1H), 6.42 (d, 1H), 6.83 (d, 2H), 7.05 (d, 2H), 7.24 (dd, 1H), 7.28-7.33 (m, 2H), 7.39 (dd, 1H).

### Example 23

### N-(2,6-dichlorobenzoyl)-4-(3-{[6-(methylamino)pyridin-2-yl]methyl}azetidin-1-yl)-L-phenylalanine

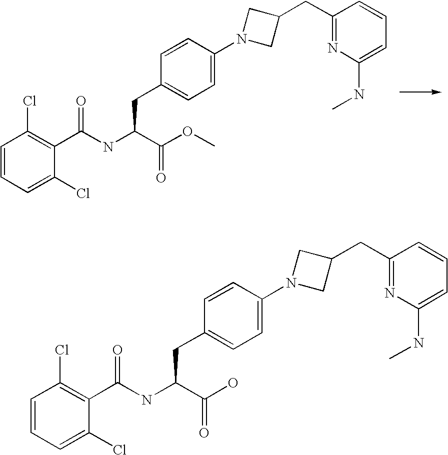

6N NaOH (0.2 ml) was added to a solution of methyl N-(2,6-dichlorobenzoyl)-4-(3-{[6-(methylamino)pyridin-2-yl]methyl}azetidin-1-yl)-L-phenylalaninate (180 mg, 0.34 mmol) in DMF (2 ml). After stirring at room temperature for 2 hours, the reaction mixture was purified by C18 reverse phase chromatography (basic conditions) to give the title compound (120 mg, 67%); ¹H NMR Spectrum: (DMSOd6) 2.74 (d, 3H), 2.83 (d, 2H) 2.84 (dd, 1H), 2.99 (dd, 1H), 3.03 (ddd, 1H), 3.48 (t, 2H), 3.84-3.91 (m, 2H), 4.45 (ddd, 1H), 6.24 (d, 1H), 6.29 (d, 2H), 6.32 (bs, 1H), 6.34 (d, 1H), 7.06 (d, 2H), 7.28 (dd, 1H), 7.38 (dd, 1H), 7.41-7.46 (m, 2H), 8.63 (bs, 1H).

The methyl N-(2,6-dichlorobenzoyl)-4-(3-{[6-(methylamino)pyridin-2-yl]methyl}azetidin-1-yl)-L-phenylalaninate used as the starting material was prepared as follows:

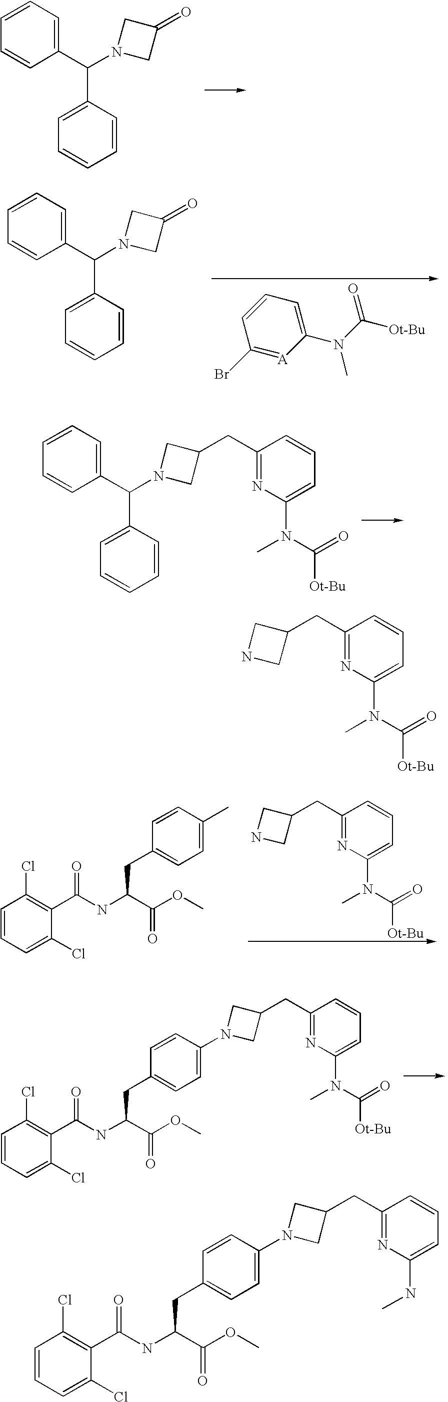

To a solution of methyl triphenylphosphoniumiodide (14.52 g, 35.2 mmol) in DMSO (30 ml) was added potassium tert-butylate (3.83 g, 34.1 mmol). The mixture was stirred at 25° C. for 75 minutes and 1-(diphenylmethyl)azetidin-3-one (2.65 g, 11.63 mmol) was added. The reaction mixture was heated at 60° C. for 18 hours and extracted with ether. The residue was purified by silica gel flash chromatography (10 to 20% diethyl ether in petroleum ether) to give 1-(diphenylmethyl)-3-methyleneazetidine (1.93 g, 72%); ¹H NMR Spectrum: (CDCl3) 3.77 (t, 4H), 4.47 (s, 1H), 4.83-4.86 (m, 2H), 7.18 (t, 2H), 7.27 (t, 4H), 7.43 (d, 4H).

9-BBN (17.8 ml, 8.88 mmol) was added under argon atmosphere to a solution of 1-(diphenylmethyl)-3-methyleneazetidine (1.9 g, 8.07 mmol) in THF (1 ml). The mixture was heated at 75° C. for 2 hours. Then tert-butyl (6-bromopyridin-2-yl)carbamate (2.088 g, 7.27 mmol), PdCl₂dppf (196 mg, 0.24 mmol), Cs₂CO₃ (3.42 g, 10.5 mmol), DMF (15 ml) and H₂O (2 ml) were added. The mixture was heated at 60° C. for 4 hours, then cooled, extracted with ethyl acetate, concentrated and purified by silica gel flash chromatography (10 to 30% ethyl acetate) in petroleum ether to give tert-butyl (6-{[1-(diphenylmethyl)azetidin-3-yl]methyl}pyridin-2-yl)methylcarbamate (2.0 g, 56%); ¹H NMR Spectrum: (CDCl3) 1.49 (s, 9H), 2.86 (t, 2H), 2.89-3.00 (m, 3H), 3.32 (s, 3H), 3.37 (t, 2H), 4.34 (s, 1H), 6.76 (d, 1H), 7.16 (t, 2H), 7.25 (t, 4H), 7.40 (d, 4H), 7.43 (d, 1H), 7.47 (dd, 1H).

A solution of tert-butyl (6-{[1-(diphenylmethyl)azetidin-3-yl]methyl}pyridin-2-yl)methylcarbamate (1.0 g, 2.25 mmol) in ethanol (100 ml) was hydrogenated for 5 hours in the presence of 20% Palladium hydroxide on carbon (100 mg). The catalyst was filtered off and the resulting mixture was purified by silica gel flash chromatography (3 to 10% NH₃/MeOH 7N in dichloromethane) to give tert-butyl [6-(azetidin-3-ylmethyl)pyridin-2-yl]methylcarbamate (320 mg, 51%); ¹H NMR Spectrum: (CDCl3) 1.51 (s, 9H), 3.01 (d, 2H), 3.14-3.25 (m, 1H), 3.36 (s, 3H), 3.52 (t, 2H), 3.74 (t, 2H), 6.79 (d, 1H), 6.47 (d, 1H), 6.51 (dd, 1H).

To tert-butyl [6-(azetidin-3-ylmethyl)pyridin-2-yl]methylcarbamate (320 mg, 1.15 mmol) in DMSO (3 ml) were added under argon atmosphere methyl N-(2,6-dichlorobenzoyl)-4-iodo-L-phenylalaninate (830 mg, 1.73 mmol), Cs₂CO₃ (750 mg, 2.31 mmol), CuI (44 mg, 0.23 mmol) and L-proline (53 mg, 0.46 mmol). The mixture was heated at 90° C. for 18 hours, extracted with ethyl acetate and purified by silica gel flash chromatography (20 to 40% ethyl acetate in petroleum ether) to give methyl 4-[3-({6-[(tert-butoxycarbonyl)(methyl)amino]pyridin-2-yl}methyl)azetidin-1-yl]-N-(2,6-dichlorobenzoyl)-L-phenylalaninate (310 g, 43%); Mass Spectrum [M+H]⁺=627; ¹H NMR Spectrum: (CDCl3) 1.52 (s, 9H), 3.06 (d, 2H), 3.11-3.23 (m, 3H), 3.38 (s, 3H), 3.58-3.63 (m, 2H), 3.75 (s, 3H), 3.98 (dt, 2H), 5.13 (ddd, 1H), 6.25 (d, 1H), 6.36 (d, 2H), 6.81 (dd, 1H), 7.03 (d, 2H), 7.25 (dd, 1H), 7.29-7.33 (m, 2H), 7.50-7.54 (m, 2H).

A solution of methyl 4-[3-({6-[(tert-butoxycarbonyl)(methyl)amino]pyridin-2-yl}methyl)azetidin-1-yl]-N-(2,6-dichlorobenzoyl)-L-phenylalaninate (290 mg, 0.46 mmol) in TFA (10 ml) was stirred at room temperature for 1 hour, then solvent was removed. The residue was dissolved in DCM and 7N NH₃/MeOH was added. After evaporation, the residue was purified by silica gel flash chromatography (3% NH₃/MeOH 7N in dichloromethane) to give methyl N-(2,6-dichlorobenzoyl)-4-(3-{[6-(methylamino)pyridin-2-yl]methyl}azetidin-1-yl)-L-phenylalaninate (200 mg, 83%); ¹H NMR Spectrum (CDCl3) 2.90 (d, 3H), 2.93 (d, 2H), 3.08-3.23 (m, 3H), 3.55-3.62 (m, 2H), 3.75 (s, 3H), 3.92-3.99 (m, 2H), 4.51 (bs, 1H), 5.13 (ddd, 1H), 6.23 (d, 1H), 6.25 (d, 1H), 6.36 (d, 2H), 6.41 (d, 1H), 7.02 (d, 2H), 7.25 (dd, 1H), 7.28-7.33 (m, 2H), 7.37 (dd, 1H).

### Example 24

### N-(2,6-dichlorobenzoyl)-4-[4-(pyridin-2-ylamino)butyl]-L-phenylalanine

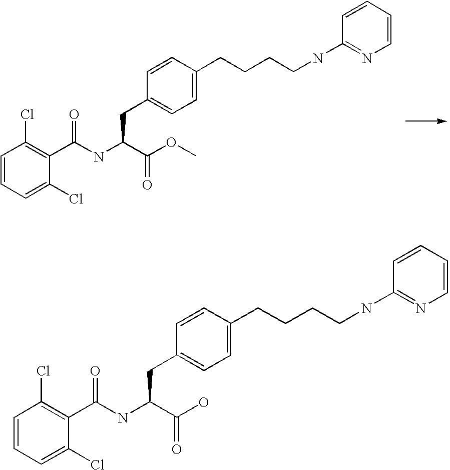

LiOH (90 mg, 3.7 mmol) was added to a solution of methyl N-(2,6-dichlorobenzoyl)-4-[4-(pyridin-2-ylamino)butyl]-L-phenylalaninate (115 mg, 0.23 mmol) in DMA/MeOH/H₂O (0.8 ml/0.1 ml/0.1 ml). After stirring at room temperature for 2 hours, the reaction mixture was purified by C18 reverse phase chromatography (acidic conditions) to give the title compound as a white solid (30 mg, 27%); ¹H NMR Spectrum: (DMSO-d6) 1.47-1.55 (m, 2H), 1.55-1.64 (m, 2H), 2.54 (t, 2H), 2.93 (dd, 1H), 3.09 (dd, 1H), 3.18-3.26 (m, 2H), 4.40-4.49 (m, 1H), 6.38-6.46 (m, 3H), 7.05 (d, 2H), 7.17 (d, 2H), 7.31 (ddd, 1H), 7.36 (dd, 1H), 7.39-7.46 (m, 2H), 7.93 (dd, 1H), 8.47 (bs, 1H).

The methyl N-(2,6-dichlorobenzoyl)-4-[4-(pyridin-2-ylamino)butyl]-L-phenylalaninate used as the starting material was prepared as follows:

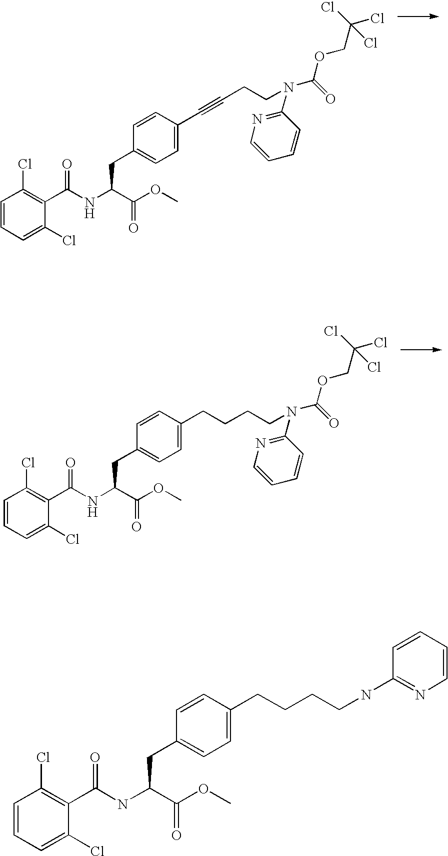

A solution of methyl methyl N-(2,6-dichlorobenzoyl)-4-(4-{pyridin-2-yl [(2,2,2-trichloroethoxy)carbonyl]amino}but-1-yn-1-yl)-L-phenylalaninate (200 mg, 0.30 mmol) described in Example 13 in methanol (6 ml) was hydrogenated under 40 psi for 5 hours in the presence of 10% Pt on carbon (40 mg). The catalyst was filtered off and the solvent was evaporated to give methyl N-(2,6-dichlorobenzoyl)-4-(4-{pyridin-2-yl [(2,2,2-trichloroethoxy)carbonyl]amino}butyl)-L-phenylalaninate which was used without further purification in next step.

To a solution of methyl N-(2,6-dichlorobenzoyl)-4-(4-{pyridin-2-yl [(2,2,2-trichloroethoxy)carbonyl]amino}butyl)-L-phenylalaninate (200 mg, 0.30 mmol) in THF (1.5 ml) were added H₂O (0.5 ml), acetic acid (1.5 ml) and Zinc (117 mg, 1.79 mmol). The mixture was stirred at room temperature overnight. After addition of ethyl acetate (20 ml), the solid was filtered and the resulting solution was purified by silica gel flash chromatography (20 to 40% ethyl acetate in DMC) to give methyl N-(2,6-dichlorobenzoyl)-4-[4-(pyridin-2-ylamino)butyl]-L-phenylalaninate (115 mg, 77%); ¹H NMR Spectrum: (DMSO-d6) 1.46-1.55 (m, 2H), 1.55-1.66 (m, 2H), 2.56 (t, 2H), 2.90 (dd, 1H), 3.08 (dd, 1H), 3.19-3.25 (m, 2H), 3.64 (s, 3H), 4.70 (ddd, 1H), 6.38-6.47 (m, 3H), 7.10 (d, 2H), 7.18 (d, 2H), 7.31 (ddd, 1H), 7.39 (dd, 1H), 7.42-7.47 (m, 2H), 7.94 (dd, 1H), 9.18 (d, 1H).

### Example 25

### N-(2,6-dichlorobenzoyl)-4-{[(5,6,7,8-tetrahydro-1,8-naphthyridin-3-ylmethyl)amino]carbonyl}-L-phenylalanine

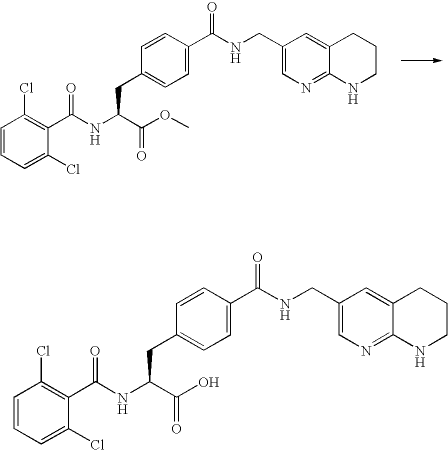

LiOH (70 mg, 2.8 mmol) was added to a solution of methyl N-(2,6-dichlorobenzoyl)-4-{[(5,6,7,8-tetrahydro-1,8-naphthyridin-3-ylmethyl)amino]carbonyl}-L-phenylalaninate (118 mg, 0.22 mmol) in DMA/MeOH/H₂O (0.7 ml/0.1 ml/0.05 ml). After stirring at room temperature for 4 hours, the reaction mixture was purified by C18 reverse phase chromatography (basic conditions) to give the title compound as a white solid (77 mg, 67%); ¹H NMR Spectrum: (DMSO-d6) 1.69-1.79 (m, 2H), 2.62 (t, 2H), 2.99 (dd, 1H), 3.20 (dd, 1H), 3.25 (bs, 2H), 4.29 (d, 2H), 4.71 (ddd, 1H), 6.32 (d, 1H), 6.39 (bs, 1H), 7.08 (d, 1H), 7.35-7.47 (m, 5H), 7.83 (d, 2H), 8.83 (t, 1H), 9.06 (d, 1H).

The methyl N-(2,6-dichlorobenzoyl)-4-{[(5,6,7,8-tetrahydro-1,8-naphthyridin-3-ylmethyl)amino]carbonyl}-L-phenylalaninate used as the starting material was prepared as follows:

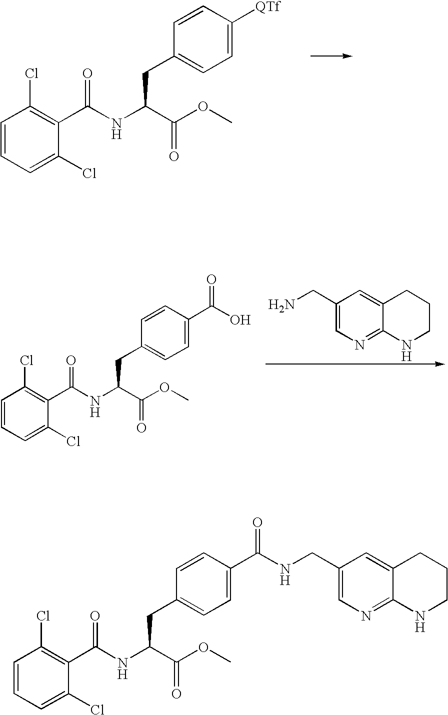

To a solution of N-(2,6-dichlorobenzoyl)-O-[(trifluoromethyl)sulfonyl]-L-tyrosinate (400 mg, 0.8 mmol) in DMF (2 ml) were added sodium formate (171 mg, 2.51 mmol), dppp (35 mg, 0.084 mmol), Palladium(II) acetate (19 mg, 0.084 mmol), DIPEA (291 μl, 1.67 mmol), acetic anhydride (177 μl, 1.67 mmol) and LiCl (105 mg, 2.51 mmol). Nitrogen was bubbled through before the reaction mixture was heated at 80° C. for 16 hours. After filtration, the residue was purified by C18 reverse phase chromatography (basic conditions) to give 4-{(2S)-2-[(2,6-dichlorobenzoyl)amino]-3-methoxy-3-oxopropyl}benzoic acid as a white solid (180 mg, 54%); ¹H NMR Spectrum: (DMSO-d6) 3.02 (dd, 1H) 3.22 (dd, 1H), 3.67 (s, 3H), 4.80 (ddd, 1H), 7.38-7.48 (m, 5H), 7.85 (m, 2H), 9.22 (d, 1H).

A solution of 4-{(2S)-2-[(2,6-dichlorobenzoyl)amino]-3-methoxy-3-oxopropyl}benzoic acid (200 mg, 0.50 mmol), 1-(5,6,7,8-tetrahydro-1,8-naphthyridin-3-yl)methanamine (82 mg, 0.50 mmol), NMP (111 μl, 1.01 mmol) and HATU (232 mg, 0.61 mmol) in DMF (2 ml) was stirred at room temperature for 2 hours. After addition of water (10 ml), the resulting precipitate was filtered, the solution was concentrated and the residue was purified by silica gel flash chromatography (0 to 5% MeOH in dichloromethane) to give methyl N-(2,6-dichlorobenzoyl)-4-{[(5,6,7,8-tetrahydro-1,8-naphthyridin-3-ylmethyl)amino]carbonyl}-L-phenylalaninate (122 mg, 45%); Mass Spectrum [M+H]⁺=541; ¹H NMR Spectrum: (DMSO-d6) 1.71-1.79 (m, 2H), 2.62 (t, 2H), 3.01 (dd, 1H), 3.21 (dd, 1H), 3.22-3.28 (m, 2H), 3.67 (s, 3H), 4.28 (d, 2H), 4.79 (ddd, 1H), 6.32 (d, 1H) 6.37 (bs, 1H), 7.08 (d, 1H), 7.38 (d, 2H) 7.39-7.47 (m, 3H), 7.83 (d, 2H), 8.84 (t, 1H), 8.23 (d, 1H).

### Example 26

### N-(2,6-dichlorobenzoyl)-O-[2-(5,6,7,8-tetrahydro-1,8-naphthyridin-2-yl)propyl]-L-tyrosine

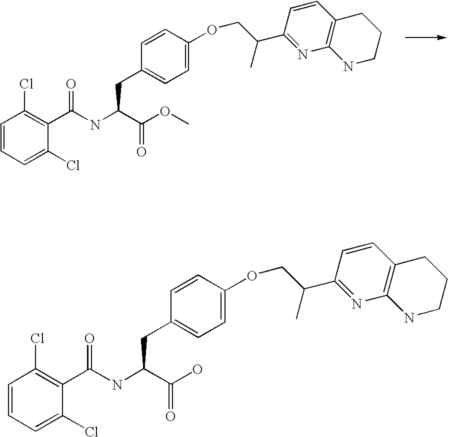

LiOH (47 mg, 1.11 mmol) was added to a solution of methyl N-(2,6-dichlorobenzoyl)-O-[2-(5,6,7,8-tetrahydro-1,8-naphthyridin-2-yl)propyl]-L-tyrosinate (200 mg, 0.37 mmol) in DMA/H₂O (2 ml/0.3 ml). After stirring at room temperature for 6 hours, the reaction mixture was purified by C18 reverse phase chromatography (basic conditions) to give the title compound as a white solid (130 mg, 67%); ¹H NMR Spectrum: (DMSOd6) 1.22 (d, 3H), 1.70-1.80 (m, 2H), 2.62 (t, 2H), 2.86 (dd, 1H), 2.96-3.07 (m, 2H), 3.23 (bs, 2H), 3.92 (ddd, 1H), 4.15 (ddd, 1H), 4.57 (ddd, 1H), 6.31-6.36 (m, 2H), 6.79 (d, 2H), 7.06 (d, 1H), 7.17 (d, 2H), 7.38 (dd, 1H), 7.41-7.45 (m, 2H), 8.92 (d, 1H).

The methyl N-(2,6-dichlorobenzoyl)-O-[2-(5,6,7,8-tetrahydro-1,8-naphthyridin-2-yl)propyl]-L-tyrosinate used as the starting material was prepared as follows:

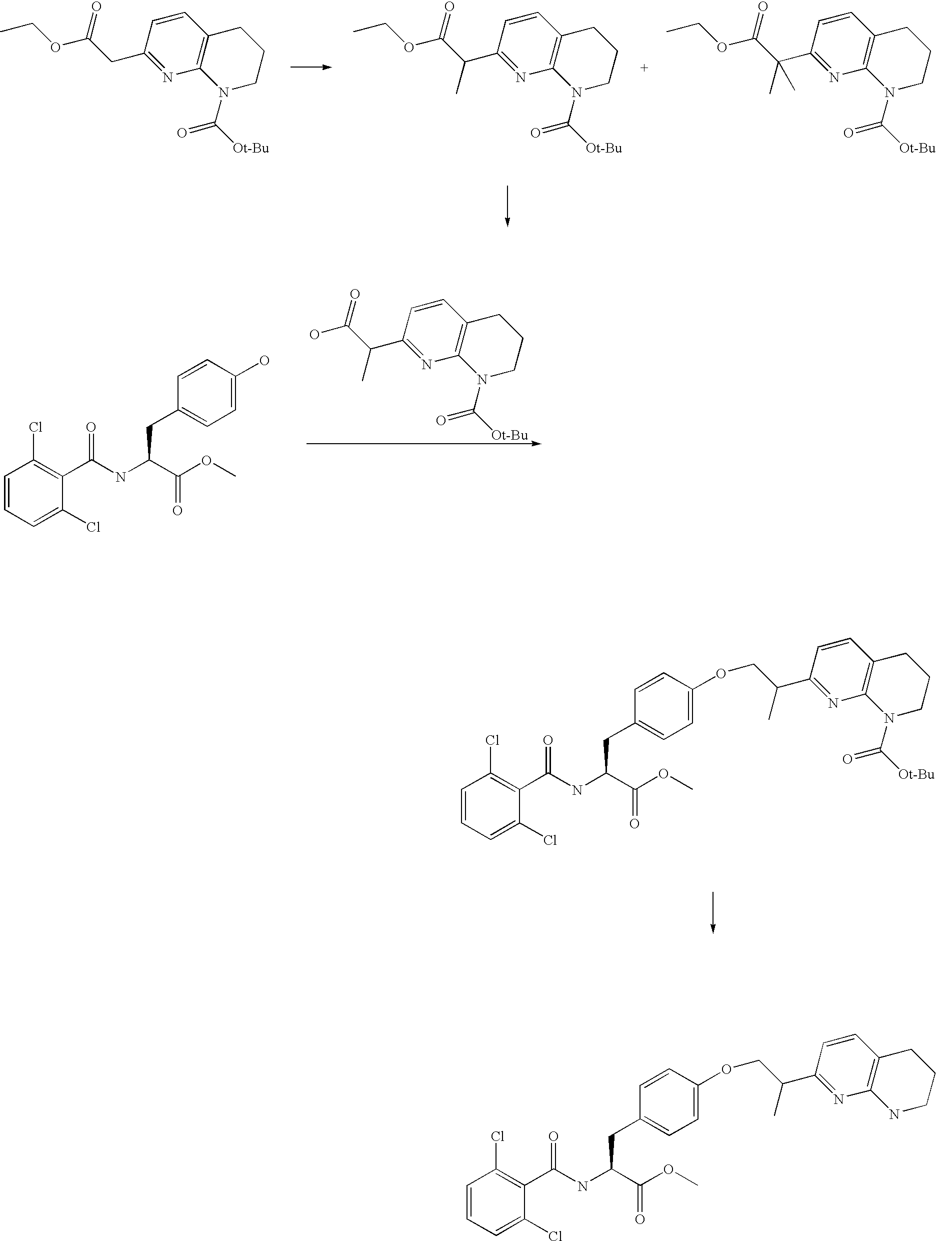

NaH (524 mg, 13.1 mmol) was added under argon atmosphere at 0° C. to a solution of tert-butyl 7-(2-ethoxy-2-oxoethyl)-3,4-dihydro-1,8-naphthyridine-1(2H)-carboxylate (3.0 g, 9.36 mmol) in THF (70 ml). The mixture was stirred for 30 minutes and methyl iodide (0.82 ml, 13.1 mmol) was added. The reaction mixture was stirred at 25° C. for 16 hours. After evaporation of the solvent, the residue was extracted with ethyl acetate. The organic layer was concentrated and purified by silica gel flash chromatography (20 to 30% ethyl acetate in petroleum ether) to give 2 separated compounds, tert-butyl 7-(2-ethoxy-1-methyl-2-oxoethyl)-3,4-dihydro-1,8-naphthyridine-1(2H)-carboxylate (1.93 g, 62%); ¹H NMR Spectrum: (CDCl3) 1.22 (t, 3H), 1.52 (s, 9H), 1.53 (d, 3H), 1.89-1.96 (m, 2H), 2.73 (t, 2H), 3.70-3.80 (m, 2H), 3.85 (q, 1H), 4.07-4.18 (m, 2H), 6.95 (d, 1H), 7.34 (d, 1H) and tert-butyl 7-(2-ethoxy-1,1-dimethyl-2-oxoethyl)-3,4-dihydro-1,8-naphthyridine-1(2H)-carboxylate (0.5 g, 15%); ¹H NMR Spectrum (DMSO-d6) 1.19 (t, 3H), 1.52 (s, 9H), 1.56 (s, 6H), 1.88-1.97 (m, 2H), 2.73 (t, 2H), 3.76 (t, 2H), 4.14 (q, 2H), 6.91 (d, 1H), 7.33 (d, 1H).

2M LiBH₄ in THF (2.25 ml, 4.5 mmol) was added at 0° C. under argon to a solution of tert-butyl 7-(2-ethoxy-1-methyl-2-oxoethyl)-3,4-dihydro-1,8-naphthyridine-1(2H)-carboxylate (1.0 g, 3.0 mmol) in THF (30 ml). The reaction mixture was stirred at 0° C. for 30 minutes, at 25° C. for 2 hours and at 70° C. for 1 hour. After hydrolysis with H₂O (0.11 ml, 6.0 mmol) at 0° C., the mixture was extracted with ethyl acetate. The organic layer was concentrated and purified by silica gel flash chromatography (30 to 50% ethyl acetate in petroleum ether) to give tert-butyl 7-(2-hydroxy-1-methylethyl)-3,4-dihydro-1,8-naphthyridine-1(2H)-carboxylate as an oil (484 mg, 56%); ¹H NMR Spectrum: (CDCl3) 1.30 (d, 3H), 1.55 (s, 9H), 1.88-1.99 (m, 2H), 2.74 (t, 2H), 3.00 (bs, 1H), 3.74-3.83 (m, 3H), 3.94 (dd, 1H), 5.47 (bs, 1H), 6.85 (d, 1H) 7.37 (bs, 1H).

To a solution of methyl N-(2,6-dichlorobenzoyl)-L-tyrosinate (210 mg, 0.57 mmol) in DMC (15 ml) were added tert-butyl 7-(2-hydroxy-1-methylethyl)-3,4-dihydro-1,8-naphthyridine-1(2H)-carboxylate (193 mg, 0.63 mmol) and triphenylphosphine (310 mg, 1.2 mmol). ADDP (303 mg, 1.2 mmol) was then added dropwise over 5 minutes and the reaction mixture was stirred at 25° C. for 18 hours. After evaporation, the residue was purified by silica gel flash chromatography (20 to 40% ethyl acetate in petroleum ether) to give tert-butyl 7-[2-(4-{(2S)-2-[(2,6-dichlorobenzoyl)amino]-3-methoxy-3-oxopropyl}phenoxy)-1-methylethyl]-3,4-dihydro-1,8-naphthyridine-1(2H)-carboxylate (0.32 g, 86%); ¹H NMR Spectrum: (CDCl3) 1.41 (d, 3H), 1.51 (s, 9H), 1.89-1.98 (m, 2H), 2.75 (t, 2H), 3.14-3.25 (m, 2H), 3.34 (bs, 1H), 3.69-3.89 (m, 2H), 3.75 (s, 3H), 4.04 (dd, 1H), 4.22 (dd, 1H), 5.11-5.17 (m, 1H), 6.26 (d, 1H), 6.81 (d, 2H), 6.94 (d, 1H), 7.08 (d, 2H), 7.24-7.33 (m, 3H), 7.38 (d, 1H).

A solution of tert-butyl 7-[2-(4-{(2S)-2-[(2,6-dichlorobenzoyl)amino]-3-methoxy-3-oxopropyl}phenoxy)-1-methylethyl]-3,4-dihydro-1,8-naphthyridine-1(2H)-carboxylate (300 mg, 0.47 mmol) in TFA (5 ml) and DCM (1 ml) was stirred at 25° C. for 5 hours. After neutralisation with 7N NH₃/MeOH and evaporation, the residue was purified by silica gel flash chromatography (3% NH₃/MeOH 7N in dichloromethane) to give methyl N-(2,6-dichlorobenzoyl)-O-[2-(5,6,7,8-tetrahydro-1,8-naphthyridin-2-yl)propyl]-L-tyrosinate (238 mg, 95%); Mass Spectrum: [M+H]⁺=542; ¹H NMR Spectrum: (CDCl3) 1.34 (d, 3H), 1.87-1.94 (m, 2H), 2.70 (t, 2H), 3.12 (q, 1H), 3.17 (dd, 1H), 3.22 (dd, 1H), 3.37-3.42 (m, 2H), 3.74 (s, 3H), 3.96 (dd, 1H) 4.18 (dd, 1H), 5.00 (bs, 1H) 5.14 (ddd, 1H), 6.26 (d, 1H) 6.42 (d, 1H), 6.81 (d, 2H), 7.07 (d, 2H), 7.10 (d, 1H), 7.26 (dd, 1H), 7.28-7.33 (m, 2H).

### Example 27

### N-(2,6-dichlorobenzoyl)-O-[2-methyl-2-(5,6,7,8-tetrahydro-1,8-naphthyridin-2-yl)propyl]-L-tyrosine

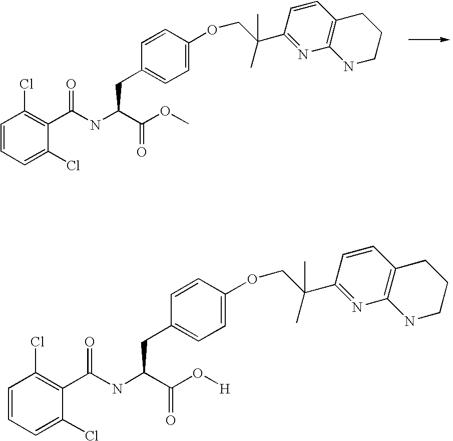

LiOH (45 mg, 1.08 mmol) was added to a solution of methyl N-(2,6-dichlorobenzoyl)-O-[2-methyl-2-(5,6,7,8-tetrahydro-1,8-naphthyridin-2-yl)propyl]-L-tyrosinate (200 mg, 0.36 mmol) in DMA/H₂O (2 ml/0.2 ml). After stirring at room temperature for 5 hours, the reaction mixture was purified by C18 reverse phase chromatography (basic conditions) to give the title compound as a white solid (150 mg, 77%); ¹H NMR Spectrum: (DMSOd6) 1.29 (s, 6H), 1.71-1.80 (m, 2H), 2.61 (t, 2H), 2.87 (dd, 1H), 3.03 (dd, 1H), 3.23 (bs, 2H), 3.98 (d, 1H), 4.01 (d, 1H), 4.52-4.59 (m, 1H), 6.17 (bs, 1H), 6.46 (d, 1H), 6.77 (d, 2H), 7.07 (d, 1H), 7.15 (d, 2H), 7.38 (dd, 1H), 7.41-7.46 (m, 2H), 8.90 (d, 1H).

The methyl N-(2,6-dichlorobenzoyl)-O-[2-methyl-2-(5,6,7,8-tetrahydro-1,8-naphthyridin-2-yl)propyl]-L-tyrosinate used as the starting material was prepared as follows:

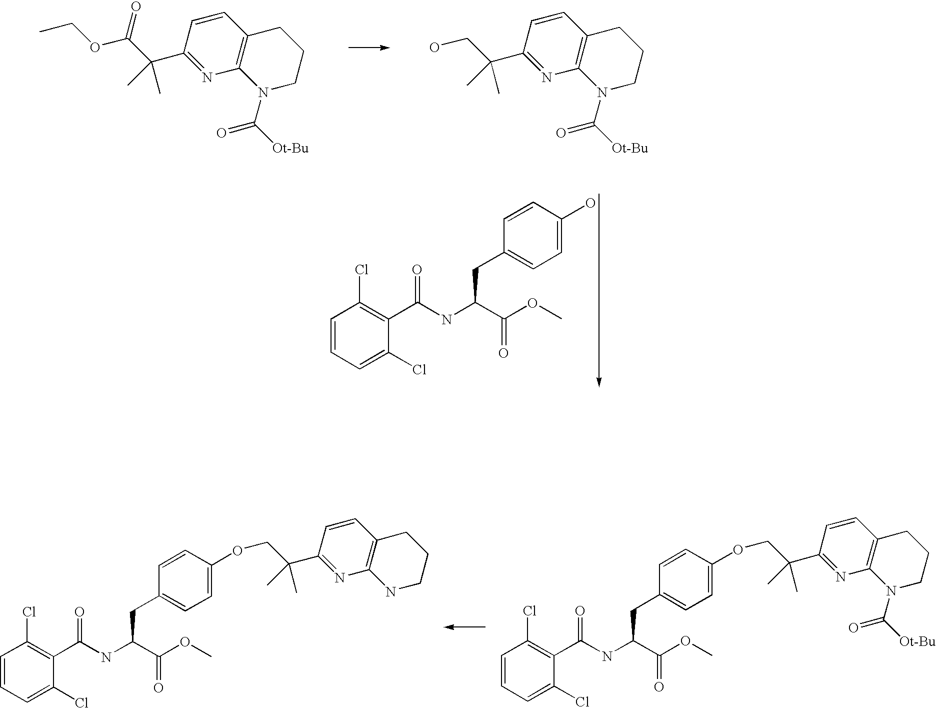

2M LiBH₄ in THF (2.0 ml, 4.0 mmol) was added at 0° C. under argon atmosphere to a solution of tert-butyl 7-(2-ethoxy-1,1-dimethyl-2-oxoethyl)-3,4-dihydro-1,8-naphthyridine-1(2H)-carboxylate (0.93 g, 2.7 mmol). The reaction mixture was stirred at 0° C. for 30 minutes, at 25° C. for 2 hours and at 70° C. for 1 hour. After hydrolysis with H₂O at 0° C., the mixture was extracted with ethyl acetate. The organic layer was concentrated and the residue purified by silica gel flash chromatography (20 to 30% ethyl acetate in petroleum ether) to give tert-butyl 7-(2-hydroxy-1,1-dimethylethyl)-3,4-dihydro-1,8-naphthyridine-1(2H)-carboxylate (597 mg, 73%).

To a solution of methyl N-(2,6-dichlorobenzoyl)-L-tyrosinate (280 mg, 0.76 mmol) in DMC (15 ml) were added tert-butyl 7-(2-hydroxy-1,1-dimethylethyl)-3,4-dihydro-1,8-naphthyridine-1(2H)-carboxylate (303 mg, 0.99 mmol), triphenylphosphine (420 mg, 1.6 mmol). ADDP (404 mg, 1.6 mmol) was then added dropwise over 5 minutes and the reaction mixture was stirred at 25° C. for 18 hours. After evaporation, the residue was purified by silica gel flash chromatography (20 to 40% ethyl acetate in petroleum ether) to give tert-butyl 7-[2-(4-{(2S)-2-[(2,6-dichlorobenzoyl)amino]-3-methoxy-3-oxopropyl}phenoxy)-1,1-dimethylethyl]-3,4-dihydro-1,8-naphthyridine-1(2H)-carboxylate (320 mg, 64%); ¹H NMR Spectrum: (CDCl3) 1.44 (s, 6H), 1.50 (s, 9H), 1.88-1.96 (m, 2H), 2.72 (t, 2H), 3.17 (dd, 1H), 3.23 (dd, 1H), 3.72-3.78 (m, 5H), 4.06 (s, 2H), 5.11-5.17 (m, 1H), 6.26 (d, 1H), 6.80 (d, 2H), 7.03 (d, 1H), 7.06 (d, 2H), 7.25 (dd, 1H), 7.29-7.34 (m, 3H).

A solution of tert-butyl 7-[2-(4-{(2S)-2-[(2,6-dichlorobenzoyl)amino]-3-methoxy-3-oxopropyl}phenoxy)-1,1-dimethylethyl]-3,4-dihydro-1,8-naphthyridine-1(2H)-carboxylate (300 mg, 0.45 mmol) in TFA (5 ml) and DCM (1 ml) was stirred at 25° C. for 5 hours. After neutralisation with 7N NH₃/MeOH and evaporation, the residue was purified by silica gel flash chromatography (3% NH₃/MeOH 7N in dichloromethane) to give methyl N-(2,6-dichlorobenzoyl)-O-[2-methyl-2-(5,6,7,8-tetrahydro-1,8-naphthyridin-2-yl)propyl]-L-tyrosinate (207 mg, 80%); Mass Spectrum [M+H]⁺=556; ¹H NMR Spectrum: (CDCl3) 1.36 (s, 6H), 1.86-1.95 (m, 2H), 2.69 (t, 2H), 3.17 (dd, 1H), 3.23 (dd, 1H), 3.39 (bs, 2H), 3.75 (s, 3H), 4.00 (bs, 2H), 4.66 (bs, 1H), 5.14 (ddd, 1H), 6.25 (d, 1H), 6.55 (d, 1H), 6.80 (d, 2H), 7.06 (d, 2H), 7.09 (d, 1H), 7.23-7.34 (m, 3H).

### Example 28.1

### N-(2-chloro-6-fluorobenzoyl)-O-[2-(3,4-dihydro-2H-pyrido[3,2-b][1,4]oxazin-6-yl)ethyl]-L-tyrosine

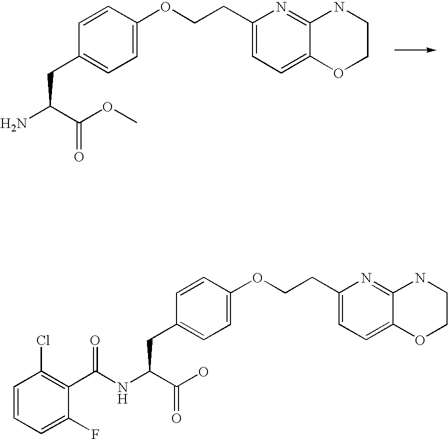

To a solution of 2-chloro-5-fluorobenzoic acid (65 mg, 0.37 mmol) in DMF (1 ml) were added TBTU (141 mg, 0.37 mmol) and methyl O-[2-(3,4-dihydro-2H-pyrido[3,2-b][1,4]oxazin-6-yl)ethyl]-L-tyrosinate (120 mg, 0.34 mmol). The mixture was stirred at room temperature for 30 hours. LiOH (62 mg, 1.34 mol) and H₂O (0.2 ml) were then added. After stirring for 12 hours, the resulting mixture was purified by C18 reverse phase chromatography (basic conditions) to give the title compound (125 mg, 74%); Mass Spectrum [M+H]⁺=500; ¹H NMR Spectrum: (DMSO-d6) 2.87 (t, 2H), 2.89 (dd, 1H), 3.06 (dd, 1H), 3.34-3.38 (m, 2H), 4.07 (t, 2H), 4.17 (t, 2H), 4.41 (dd, 1H), 6.41 (d, 1H), 6.65 (bs, 1H), 6.78 (d, 2H), 6.84 (d, 1H), 7.15 (d, 2H), 7.24 (t, 1H), 7.30 (d, 1H), 7.43 (ddd, 1H), 8.57 (bs, 1H).

The procedure described above for example 28.1 was repeated using the appropriate benzoic acid. Thus were obtained the compounds shown in Table 7:

TABLE 7

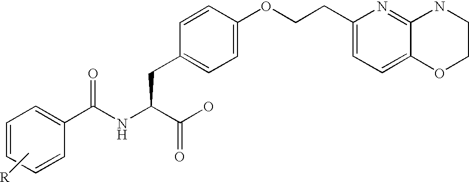

| | | | | Mass | ¹H NMR Data |
|---|---|---|---|---|---|
| Compound | R | Name | Yield | Ion | (500 MHz) |
| 28.2 | 2-chloro | N-(2- | 71 | 482 | (DMSO-d6 + TFAd) |
| | | chlorobenzoyl)- | | [M + H]⁺ | 2.93 (dd, 1H), 3.10- |
| | | O-[2-(3,4- | | | 3.20 (m, 3H), 3.62 (t, |
| | | dihydro-2H- | | | 2H), 4.21-4.32 (m, |
| | | pyrido[3,2- | | | 4H), 4.60 (dd, 1H), |
| | | b][1,4]oxazin- | | | 6.77 (d, 1H), 6.90 (d, |
| | | 6-yl)ethyl]-L- | | | 2H), 7.25 (d, 2H), 7.29 |
| | | tyrosine | | | (d, 1H), 7.38 (ddd, |
| | | | | | 1H), 7.40-7.49 (m, 3H) |
| 28.3 | 2-chloro | N-(2-chloro- | 76 | 500 | (DMSO-d6 + TFAd) |
| | 4-fluoro | 4- | | [M + H]⁺ | 2.92 (dd, 1H), 3.10- |
| | | fluorobenzoyl)- | | | 3.18 (m, 3H), 3.61 (t, |
| | | O-[2-(3,4- | | | 2H), 4.22-4.30 (m, |
| | | dihydro-2H- | | | 4H), 4.58 (dd, 1H), |
| | | pyrido[3,2- | | | 6.77 (d, 1H), 6.89 (d, |
| | | b][1,4]oxazin- | | | 2H), 7.21-7.39 (m, |
| | | 6-yl)ethyl]-L- | | | 3H), 7.35 (dd, 1H), |
| | | tyrosine | | | 7.42 (d, 2H) |
| 28.4 | 2-chloro- | N-(2-chloro- | 85 | 514 | (DMSO-d6 + TFAd) |
| | 6-fluoro- | 6-fluoro-3 - | | [M + H]⁺ | 2.30 (s, 3H), 2.89 (dd, |
| | 3-methyl | methylbenzoyl)- | | | 1H), 3.09 (dd, 1H), |
| | | O-[2-(3,4- | | | 3.14 (t, 2H), 3.61 (t, |
| | | dihydro-2H- | | | 2H), 4.20-4.32 (m, |
| | | pyrido[3,2- | | | 4H), 4.62 (dd, 1H), |
| | | b][1,4]oxazin- | | | 6.78 (d, 1H), 6.88 (d, |
| | | 6-yl)ethyl]-L- | | | 2H), 7.14 (t, 1H), 7.23 |
| | | tyrosine | | | (d, 2H), 7.40 (t, 1H), |
| | | | | | 7.43 (d, 1H) |
| 28.5 | 2,6- | N-(2-chloro- | | 516 | (DMSO-d6 + TFAd) |
| | dichoro | 6- | | [M + H]⁺ | 2.87 (t, 2H), 2.94 (dd, |
| | | fluorobenzoyl)- | | | 1H), 3.05 (dd, 1H), 3.34- |
| | | O-[2-(3,4- | | | 3.38 (m, 2H), 4.06 (t, |
| | | dihydro-2H- | | | 2H), 4.16 (t, 2H), 4.41 |
| | | pyrido[3,2- | | | (dd, 1H), 6.40 (d, 1H), |
| | | b][1,4]oxazin- | | | 6.66 (s, 1H), 6.76 (d, |
| | | 6-yl)ethyl]-L- | | | 2H), 6.84 (d, 1H), 7.15 |
| | | tyrosine | | | (d, 2H), 7.37 (dd, 1H), |
| | | | | | 7.40-7.46 (m, 2H), 8.17 |
| | | | | | (bs, 1H) |

The starting material methyl O-[2-(3,4-dihydro-2H-pyrido[3,2-b][1,4]oxazin-6-yl)ethyl]-L-tyrosinate was prepared as follows:

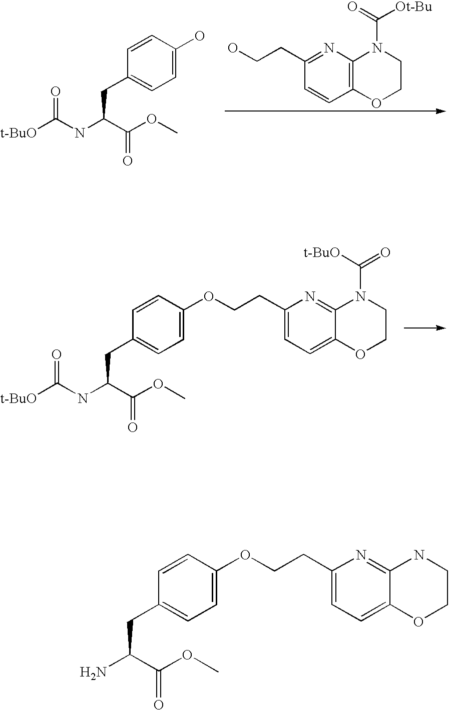

To a solution of methyl N-(tert-butoxycarbonyl)-L-tyrosinate (250 mg, 0.85 mmol) in DMC (4 ml) were added at −20° C. methyl O-[2-(3,4-dihydro-2H-pyrido[3,2-b][1,4]oxazin-6-yl)ethyl]-L-tyrosinate (261 mg, 0.93 mmol) and triphenylphosphine (267 mg, 1.02 mmol). ADDP (320 mg, 1.27 mmol) was then added dropwise over 5 minutes and the reaction mixture was stirred at room temperature overnight. After evaporation, the residue was purified by silica gel flash chromatography (10 to 50% ethyl acetate in petroleum ether) to give tert-butyl 6-[2-(4-{(2S)-2-[(tert-butoxycarbonyl)amino]-3-methoxy-3-oxopropyl}phenoxy)ethyl]-2,3-dihydro-4H-pyrido[3,2-b][1,4]oxazine-4-carboxylate (338 mg, 72%); ¹H NMR Spectrum: (CDCl3) 1.41 (s, 9H), 1.53 (s, 9H), 2.94-3.09 (m, 2H), 3.15 (t, 2H), 3.70 (s, 3H), 3.91 (t, 2H), 4.23 (t, 2H), 4.30 (t, 2H), 4.53 (dd, 1H), 4.94 (d, 1H), 6.83 (d, 2H), 6.93 (d, 1H), 7.01 (d, 2H), 7.10 (d, 1H).

A solution of tert-butyl 6-[2-(4-{(2S)-2-[(tert-butoxycarbonyl)amino]-3-methoxy-3-oxopropyl}phenoxy)ethyl]-2,3-dihydro-4H-pyrido[3,2-b][1,4]oxazine-4-carboxylate (335 mg, 0.60 mmol) in TFA (1 ml) and DCM (2 ml) was stirred at 25° C. for 12 hours. After evaporation and neutralisation with 0.5 N NaOH the residue was extracted with ethyl acetate. Evaporation to dryness gave tert-butyl 6-[2-(4-{(2S)-2-[(tert-butoxycarbonyl)amino]-3-methoxy-3-oxopropyl}phenoxy)ethyl]-2,3-dihydro-4H-pyrido[3,2-b][1,4]oxazine-4-carboxylate (172 mg, 80%); ¹H NMR Spectrum: (CDCl3) 2.80 (dd, 1H), 2.98-306 (m, 3H), 3.52-3.57 (m, 2H), 3.68 (dd, 1H), 3.71 (s, 3H), 4.20 (t, 2H), 4.23 (t, 2H), 4.84 (bs, 1H), 6.50 (d, 1H), 6.84 (d, 2H), 6.90 (d, 1H), 7.07 (d, 1H).

### Example 29

### N-(2,6-dichlorobenzoyl)-3-(5-{3-[6-(methylamino)pyridin-2-yl]propyl}-2-thienyl)-L-alanine

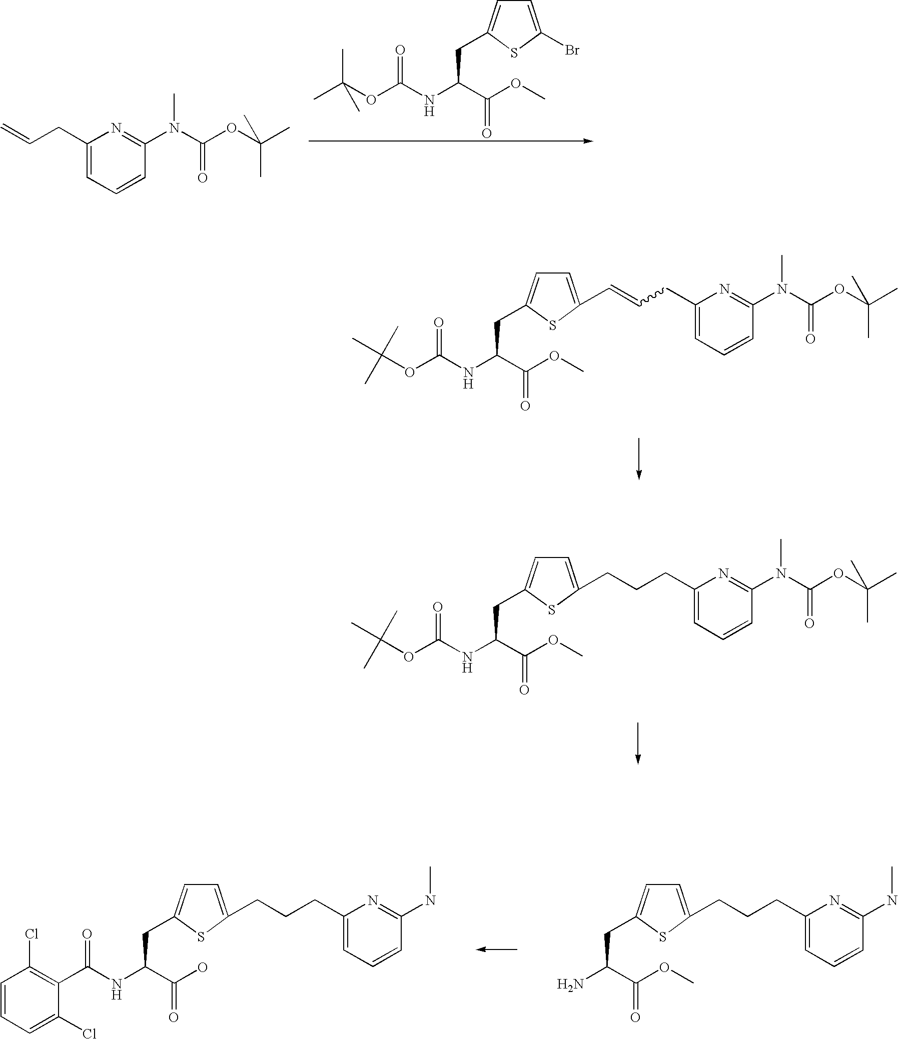

To a solution of methyl N-(2,6-dichlorobenzoyl)-3-(5-{3-[6-(methylamino)pyridin-2-yl]propyl}-2-thienyl)-L-alaninate (350 mg, 0.69 mmol) in DMF (10 ml) was added NaOH 6M (0.2 ml). After 3 hours at room temperature, the crude mixture was filtered and purified by C18 reverse phase chromatography (basic conditions) to afford the title compound as a pale yellow solid (222 mg, 65%); Mass Spectrum [M+H]⁺=492; ¹H NMR Spectrum (DMSO-d6) 1.87-1.96 (m, 2H), 2.54 (t, 2H), 2.71 (t, 2H), 2.74 (d, 3H), 3.14 (dd, 1H), 3.25 (dd, 1H), 4.42 (bs, 1H), 6.22 (d, 1H), 6.30 (q, 1H), 6.33 (d, 1H), 6.61 (d, 1H), 6.70 (d, 1H), 7.27 (dd, 1H), 7.39 (dd, 1H), 7.42-7.46 (m, 2H), 8.57 (bs, 1H).

The methyl N-(2,6-dichlorobenzoyl)-3-(5-{3-[6-(methylamino)pyridin-2-yl]propyl}-2-thienyl)-L-alaninate used as the starting material was prepared as follows:

A solution of methyl 3-(5-bromo-2-thienyl)-N-(tert-butoxycarbonyl)-L-alaninate (1.2 g, 3.3 mmol) described in USP 2005171148, tert-butyl (6-allylpyridin-2-yl)methylcarbamate (Example 18 above) (817 mg, 3.3 mmol), Palladium(II) acetate (222 mg, 1.0 mmol), tri-O-tolylphosphine (401 mg, 1.3 mmol) and DIPEA (0.57 ml, 3.3 mmol) in acetonitrile (4 ml) was degassed, then sealed and heated to 110° C. for 3 hours in microwave. The reaction mixture was cooled down, filtered to remove the catalyst and purified by silica gel flash chromatography (0 to 20% ethyl acetate in petroleum ether) to afford methyl N-(tert-butoxycarbonyl)-3-[5-(3-{6-[(tert-butoxycarbonyl)(methyl)amino]pyridin-2-yl}prop-1-en-1-yl)-2-thienyl]-L-alaninate as a cis-trans mixture (640 mg, 37%); Mass Spectrum [M+H]⁺=532.

A mixture of methyl N-(tert-butoxycarbonyl)-3-[5-(3-{6-[(tert-butoxycarbonyl)(methyl)amino]pyridin-2-yl}prop-1-en-1-yl)-2-thienyl]-L-alaninate (640 mg, 1.20 mmol) and Pd/C 10% (120 mg) in ethanol (30 ml) was hydrogenated under 3 bars of H₂ for 3 hours, filtered through celite and concentrated to afford methyl N-(tert-butoxycarbonyl)-3-[5-(3-{6-[(tert-butoxycarbonyl)(methyl)amino]pyridin-2-yl}propyl)-2-thienyl]-L-alaninate as a colorless oil (640 mg, 99%); ¹H NMR Spectrum (DMSO-d6) 1.35 (s, 9H), 1.45 (s, 9H), 1.92-2.02 (m, 2H), 2.72 (t, 2H), 2.75 (t, 2H), 3.02 (dd, 1H), 3.15 (dd, 1H), 3.28 (s, 3H), 3.62 (s, 3H), 4.13 (ddd, 1H), 6.65 (d, 1H), 6.69 (d, 1H), 6.97 (d, 1H), 7.32 (d, 1H), 7.41 (d, 1H), 7.65 (dd, 1H).

TFA (5 ml) was added to a solution of methyl N-(tert-butoxycarbonyl)-3-[5-(3-{6-[(tert-butoxycarbonyl)(methyl)amino]pyridin-2-yl}propyl)-2-thienyl]-L-alaninate (640 mg, 1.22 mmol) in dichloromethane (4 ml). The reaction mixture was stirred 1 hour at room temperature, then solvent was removed. The residue was dissolved in dichloromethane and NH₃/MeOH 7N was added. Filtration and evaporation were followed by a purification by silica gel flash chromatography (0 to 5% MeOH in dichloromethane) to afford methyl 3-(5-{3-[6-(methylamino)pyridin-2-yl]propyl}-2-thienyl)-L-alaninate as a gum (350 mg, 88%); Mass Spectrum [M+H]⁺=334; ¹H NMR Spectrum (DMSO-d6) 1.88-1.96 (m, 2H), 1.97 (bs, 2H), 2.54 (t, 2H), 2.73 (d, 3H), 2.93 (dd, 1H), 3.01 (dd, 1H), 3.53 (t, 1H), 3.60 (s, 3H), 6.22 (d, 1H), 6.29 (q, 1H), 6.32 (d, 1H), 6.62-6.65 (m, 2H), 7.27 (dd, 1H).

To a solution of methyl 3-(5-{3-[6-(methylamino)pyridin-2-yl]propyl}-2-thienyl)-L-alaninate (350 mg, 1.05 mmol) in DMF (9 ml) were added N-Methyl morpholine (0.15 ml, 1.36 mmol) and 2,6-dichlorobenzoic acid (201 mg, 1.05 mmol) and TBTU (405 mg, 1.26 mmol). The reaction mixture was allowed to stir at room temperature overnight. The crude mixture was filtered and purified by C18 reverse phase chromatography (basic conditions) to afford methyl N-(2,6-dichlorobenzoyl)-3-(5-{3-[6-(methylamino)pyridin-2-yl]propyl}-2-thienyl)-L-alaninate (350 mg, 66%); Mass Spectrum [M+H]⁺=506; ¹H NMR Spectrum (DMSO-d6) 1.88-1.96 (m, 2H), 2.54 (t, 2H), 2.74 (d, 3H), 2.75 (t, 2H), 3.13 (dd, 1H), 3.25 (dd, 1H), 3.66 (s, 3H), 4.68 (ddd, 1H), 6.22 (d, 1H), 6.29 (q, 1H), 6.32 (d, 1H), 6.65 (d, 1H), 6.74 (d, 1H), 7.24 (dd, 1H), 7.41 (dd, 1H), 7.44-7.48 (m, 2H), 9.25 (d, 1H).

### Example 3

### N-[(2,6-dichlorophenyl)carbonothioyl]-4-{3-[6-(methylamino)pyridin-2-yl]propyl}-L-phenylalanine

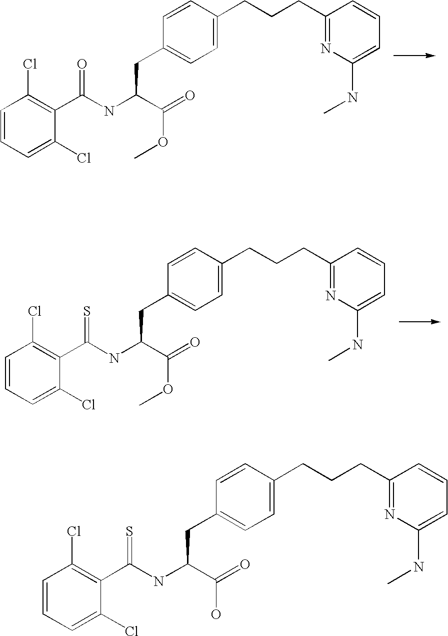

To a solution of methyl N-[(2,6-dichlorophenyl)carbonothioyl]-4-{3-[6-(methylamino)pyridin-2-yl]propyl}-L-phenylalaninate (147 mg, 0.28 mmol) in DMF (4 ml) was added NaOH 6N (0.3 ml). The reaction mixture was allowed to stir at 40° C. for 2 hours, then water (1 ml) was added, and the mixture was directly purified by C18 reverse phase chromatography (basic conditions) to afford the title compound (103 mg, 70%); Mass Spectrum [M+H]⁺=502; ¹H NMR Spectrum (DMSOd6) 1.84-1.94 (m, 2H), 2.50 (t partially hidden by DMSOd5, 2H), 2.56 (t, 2H), 2.74 (d, 3H), 3.07 (dd, 1H), 3.21 (dd, 1H), 5.26 (bs, 1H), 6.22 (d, 1H), 6.29 (q, 1H), 6.32 (d, 1H), 7.11 (d, 2H), 7.21 (d, 2H), 7.28 (dd, 1H), 7.32 (d, 1H), 7.38 (dd, 1H), 7.44 (dd, 1H), 10.99 (bs, 1H).

The starting methyl N-[(2,6-dichlorophenyl)carbonothioyl]-4-{3-[6-(methylamino)pyridin-2-yl]propyl}-L-phenylalaninate was made as follows:

To a solution of methyl N-(2,6-dichlorobenzoyl)-4-{3-[6-(methylamino)pyridin-2-yl]propyl}-L-phenylalaninate (820 mg, 1.64 mmol), prepared in example 18.1, in dry toluene (20 ml) was added Lawesson reagent (1.33 g, 3.28 mmol). The reaction mixture was degassed, sealed and allowed to stir at reflux for 48 hours. After evaporation, the residue was purified by C18 reverse phase chromatography (basic conditions) to afford methyl N-[(2,6-dichlorophenyl)carbonothioyl]-4-{3-[6-(methylamino)pyridin-2-yl]propyl}-L-phenylalaninate (147 mg, 17%); Mass Spectrum [M+H]⁺=516; ¹H NMR Spectrum (CDCl3) 1.92-2.05 (m, 2H), 2.57-2.69 (m, 4H), 2.88 (d, 3H), 3.33 (dd, 1H), 3.41 (dd, 1H), 3.77 (s, 3H), 4.77 (ms, 1H), 5.59-5.66 (m, 1H), 6.21 (d, 1H), 6.43 (d, 1H), 7.11 (bs, 4H), 7.20 (dd, 1H), 7.28-7.33 (m, 2H), 7.38 (dd, 1H), 7.85 (d, 1H).

### Example 31

### N-[(2,6-dichlorophenyl)carbonyl]-4-({[2-(pyridin-2-ylamino)ethyl]amino}methyl)-L-phenylalanine

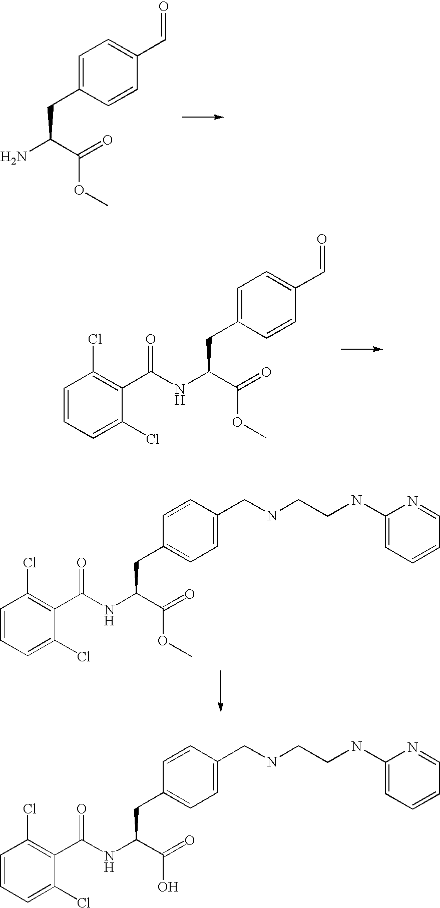

To a solution of methyl N-[(2,6-dichlorophenyl)carbonyl]-4-({[2-(pyridin-2-ylamino)ethyl]amino}methyl)-L-phenylalaninate (60 mg, 0.12 mmol) in DMA/H₂O (0.8 ml/0.2 ml) was added LiOH (30 mg). The reaction mixture was stirred at room temperature for 3 hours, then directly purified by C18 reverse phase chromatography (basic conditions) to afford the title compound as a white solid (15 mg, 26%); Mass Spectrum [M+H]⁺=487; ¹H NMR Spectrum (DMSO-d6)) 2.78-2.84 (m, 2H), 3.04 (dd, 1H), 3.14 (dd, 1H), 3.38-3.44 (m partially hidden by H2O, 2H), 3.85 (s, 2H), 4.48 (dd, 1H), 6.43-6.48 (m, 2H), 6.54-6.60 (m, 1H), 7.26 (d, 2H), 7.29 (d, 2H), 7.32-7.40 (m, 2H), 7.41-7.44 (m, 2H), 7.92 (dd, 1H), 8.43 (bs, 1H)

The starting material methyl N-[(2,6-dichlorophenyl)carbonyl]-4-({[2-(pyridin-2-ylamino)ethyl]amino}methyl)-L-phenylalaninate was prepared as follows:

To a solution of methyl N-(tert-butoxycarbonyl) 4-formyl-L-phenylalaninate (500 mg, 1.63 mmol) described in Synthetic Comm. 1998, 28(22), 4279 in DCM (2 ml) was added TFA (2 ml). The reaction mixture was stirred at room temperature for 1.5 hours. Evaporation to dryness gave crude methyl 4-formyl-L-phenylalaninate as a TFA salt, which was used in next step without further purification.

To a solution of methyl 4-formyl-L-phenylalaninate (400 mg, 1.93 mmol) triethylamine (0.54 ml, 3.86 mmol) in DCM (8 ml) was added dropwise 2,6-dichlorobenzoic acid (0.19 ml, 1.35 mmol) at 0° C. After 2 hours at 0° C., a few drops of water were added and the reaction mixture was allowed to warm up to room temperature. The mixture was purified through a plug of silica gel, eluting with DCM, to give methyl N-[(2,6-dichlorophenyl)carbonyl]-4-formyl-L-phenylalaninate (270 mg, 37%); Mass Spectrum [M+H]⁺=380; ¹H NMR Spectrum (DMSO-d6) 3.05 (dd, 1H), 3.27 (dd, 1H), 3.68 (s, 3H), 4.83 (ddd, 1H), 7.38-7.48 (m, 3H), 7.53 (d, 2H), 7.84 (d, 2H), 9.24 (d, 1H), 9.98 (s, 1H)

To a solution of methyl N-[(2,6-dichlorophenyl)carbonyl]-4-formyl-L-phenylalaninate (150 mg, 0.39 mmol) in DCM (2 ml) were successively added at room temperature AcOH (801), 2-(2-pyridylamino)ethylamine (Eur. J. Med. Chem. Chim. Ther. 1987, 22, 91)(81 mg, 0.59 mmol) and 5 seeds of 5 Å molecular sieves. After 15 minutes, NaBH(OAc)₃ (125 mg, 0.59 mmol) was added. The reaction was stirred at ambient temperature for 3 hours. A few drops of water were added, and solvents were removed under vacuum. The residue was dissolved in DMA and purified by C18 reverse phase chromatography (acidic conditions) to afford methyl N-[(2,6-dichlorophenyl)carbonyl]-4-({[2-(pyridin-2-ylamino)ethyl]amino}methyl)-L-phenylalaninate (60 mg, 30%); Mass Spectrum [M+H]⁺=501; ¹H NMR Spectrum (DMSO-d6) 2.90 (bs, 2H), 2.95 (dd, 1H), 3.13 (dd, 1H), 3.42-3.46 (m partially hidden by H2O, 2H), 3.65 (s, 3H), 3.97 (bs, 2H), 4.72 (ddd, 1H), 7.47-7.54 (m, 2H), 6.63 (bs, 1H), 7.30 (d, 2H), 7.34 (d, 2H), 7.36-7.47 (m, 4H), 7.94 (dd, 1H), 9.23 (d, 1H).

### Example 32

### N-[(2,6-dichlorophenyl)carbonyl]-O-{3-[(5-methoxypyridin-2-yl)amino]propyl}-L-tyrosine

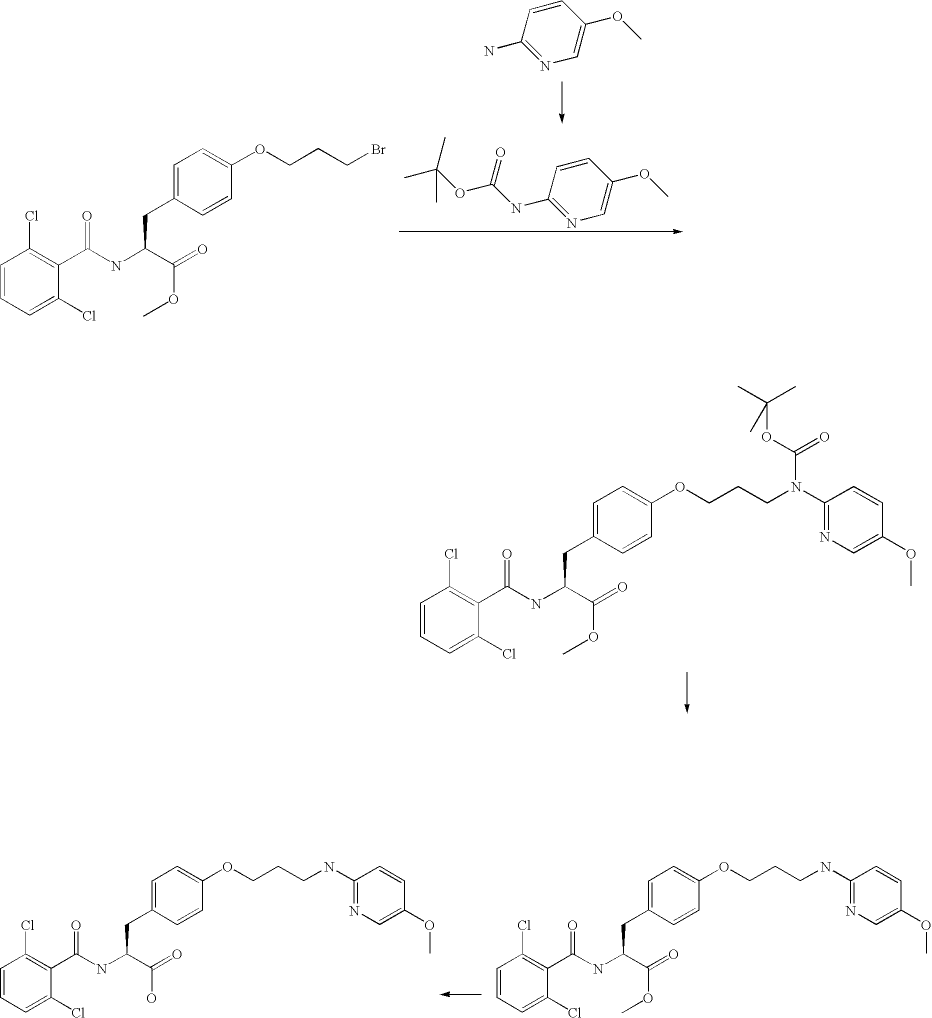

NaOH 6N (0.17 ml, 1.0 mmol) was added to a solution of methyl N-[(2,6-dichlorophenyl)carbonyl]-O-{3-[(5-methoxypyridin-2-yl)amino]propyl}-L-tyrosinate (180 mg, 0.34 mmol) in DMF (1.0 ml). After 1.5 hours at room temperature, 0.3 ml of water were added and the crude reaction mixture was purified by C18 reverse phase chromatography (basic conditions) to afford the title compound as a white solid (116 mg, 64%); Mass Spectrum [M+H]⁺=518; ¹H NMR Spectrum (DMSO-d6) 1.90-1.99 (m, 2H), 2.92 (dd, 1H), 3.07 (dd, 1H), 3.28-3.36 (m, 2H), 3.68 (s, 3H), 4.00 (t, 2H), 4.44 (ddd, 1H), 6.18 (bs, 1H), 6.42 (d, 1H), 6.80 (d, 2H), 7.12 (dd, 1H), 7.18 (d, 2H), 7.38 (dd, 1H), 7.41-7.46 (m, 2H), 7.72 (d, 1H), 8.44 (d, 1H)

The starting material methyl N-[(2,6-dichlorophenyl)carbonyl]-O-{3-[(5-methoxypyridin-2-yl)amino]propyl}-L-tyrosinate was prepared as follows: To a solution of 1.0M LiHMDS (35.4 ml, 35.4 mmol) in THF (15 ml) at −30° C. was slowly added dropwise a solution of 2-amino-5-methoxypyridine (2.0 g, 16.1 mmol) in THF (15 ml). After 1 hour at −30° C., a solution of BOC₂O (3.69 g, 16.9 mmol) in THF (20 ml) was slowly added dropwise. The reaction mixture was then stirred at 0° C. for 2 hours until completion. The reaction mixture was quenched with HCl 1N (25 ml) and partially concentrated, then the aqueous layer was extracted twice with ethyl acetate. The organic layer was dried, concentrated and purified by silica gel flash chromatography (10 to 20% ethyl acetate in petroleum ether) to give tert-butyl (5-methoxypyridin-2-yl)carbamate as a white solid (2.52 g, 70%); ¹H NMR Spectrum (CDCl3) 1.53 (s, 9H), 3.83 (s, 3H), 7.25 (dd, 1H), 7.56 (bs, 1H), 7.88 (d, 1H), 7.93 (d, 1H).

To a solution of tert-butyl (5-methoxypyridin-2-yl)carbamate (200 mg, 0.89 mmol) in DMF (6 ml) was added sodium hydride (60% in oil, 38 mg, 0.94 mmol) at 0° C. Temperature was raised to room temperature for 60 minutes, then methyl O-(3-bromopropyl)-N-(2,6-dichlorobenzoyl)-L-tyrosinate (example 1.2a) was added (460 mg, 0.94 mmol) at 0° C., and the reaction mixture was stirred at room temperature for 1.5 hours. The reaction mixture was concentrated, then diluted in ethyl acetate, washed with water and dried with MgSO₄. After concentration, a purification by silica gel flash chromatography (20 to 45% ethyl acetate in petroleum ether) afforded methyl O-{3-[(tert-butoxycarbonyl)(5-methoxypyridin-2-yl)amino]propyl}-N-[(2,6-dichlorophenyl)carbonyl]-L-tyrosinate as a white foam (290 mg, 52%); Mass Spectrum [M+H]⁺=632; ¹H NMR Spectrum (CDCl3)) 1.45 (s, 9H), 2.03-2.11 (m, 2H), 3.18 (dd, 1H), 3.22 (dd, 1H), 3.75 (s, 3H), 3.82 (s, 3H), 3.95 (t, 2H), 4.02 (t, 2H), 5.14 (ddd, 1H), 6.29 (d, 1H), 6.74 (d, 2H), 7.07 (d, 2H), 7.14 (dd, 1H), 7.26 (dd, 1H), 7.29-7.34 (m, 2H), 7.38 (d, 1H), 8.02 (d, 1H).

Methyl O-{3-[(tert-butoxycarbonyl)(5-methoxypyridin-2-yl)amino]propyl}-N-[(2,6-dichlorophenyl)carbonyl]-L-tyrosinate (250 mg, 0.40 mmol) in DCM (1 ml) was stirred with TFA (5 ml) for 1.5 hours at room temperature. Evaporation was followed by dissolution in DCM and slow addition of a solution of NH₃ 7N in MeOH at 0° C. Removal of salts by filtration was followed by a purification by silica gel flash chromatography (2% NH₃/MeOH 7N in DCM) to give methyl N-[(2,6-dichlorophenyl)carbonyl]-O-{3-[(5-methoxypyridin-2-yl)amino]propyl}-L-tyrosinate as a beige foam (210 mg, 100%); Mass Spectrum [M+H]⁺=532; ¹H NMR Spectrum (CDCl3) 2.04-2.12 (m, 2H), 3.17-3.25 (m, 2H), 3.23 (dd, 1H), 3.75 (s, 3H), 3.77 (s, 3H), 4.04 (t, 2H), 4.46 (bs, 1H), 5.15 (ddd, 1H), 6.32 (d, 1H), 6.38 (d, 1H), 6.81 (d, 2H), 7.09 (dd, 1H), 7.11 (d, 2H), 7.25 (dd, 1H), 7.29-7.33 (m, 2H), 7.79 (d, 1H).

### Example 33

### N-[(2,6-dichlorophenyl)carbonyl]-O-{3-[(4-methoxypyridin-2-yl)amino]propyl}-L-tyrosine

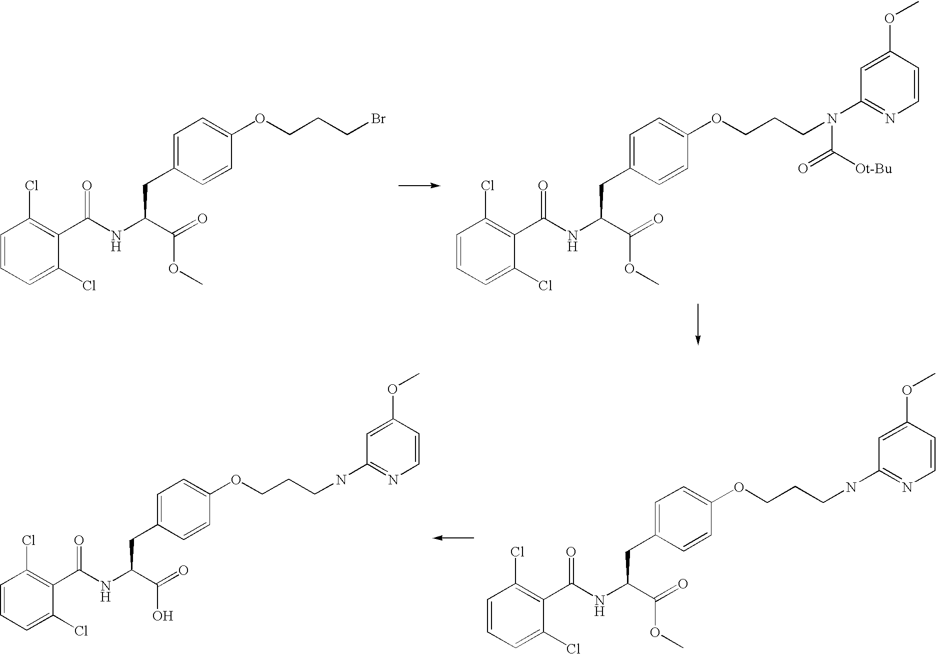

To a solution of methyl O-{3-[(tert-butoxycarbonyl)(4-methoxypyridin-2-yl)amino]propyl}-N-[(2,6-dichlorophenyl)carbonyl]-L-tyrosinate (95 mg 0.19 mm) in DCM (1 ml) was added TFA (1 ml). The reaction mixture was stirred at room temperature for 2 hours. Addition of NH₃ 7M in MeOH until neutral pH was followed by filtration of the salts and evaporation to dryness. The residue was dissolved in DMF/H₂O (1 ml/0.3 ml) and LiOH (30 mg) was added. After 3 hours at room temperature, the reaction mixture was filtered and purified by C18 reverse phase chromatography (basic conditions) to afford the title compound as a white solid (39 mg, 42%); Mass Spectrum [M+H]⁺=518; ¹H NMR Spectrum (DMSO-d6) 1.89-1.99 (m, 2H), 2.88 (dd, 1H), 3.05 (dd, 1H), 3.32-3.38 (m partially hidden by H2O, 2H), 3.70 (s, 3H), 4.00 (t, 2H), 4.55 (ddd, 1H), 5.95 (d, 1H), 6.11 (dd, 1H), 6.48 (t, 1H), 6.82 (d, 2H), 7.18 (d, 2H), 7.38 (dd, 1H), 7.41-7.46 (m, 2H), 7.78 (d, 1H), 8.86 (d, 1H).

The starting material methyl O-{3-[(tert-butoxycarbonyl)(4-methoxypyridin-2-yl)amino]propyl}-N-[(2,6-dichlorophenyl)carbonyl]-L-tyrosinate was prepared as follows:

To a solution of 2-Boc-amino-4-methoxypyridine (110 mg, 0.49 mmol) in DMF (1 ml) was slowly added at 0° C. sodium hydride (60%, 20 mg, 0.49 mmol) suspended in DMF (1 ml). After 30 minutes, methyl O-(3-bromopropyl)-N-(2,6-dichlorobenzoyl)-L-tyrosinate (200 mg, 0.41 mmol) was added. The reaction mixture was allowed to warm up to room temperature and to stir for 4 hours. A few drops of aqueous saturated ammonium chloride were added, then filtration of salts was followed by a purification by C18 reverse phase chromatography (basic conditions) to afford methyl O-{3-[(tert-butoxycarbonyl)(4-methoxypyridin-2-yl)amino]propyl}-N-[(2,6-dichlorophenyl)carbonyl]-L-tyrosinate as a colorless oil (95 mg, 37%); Mass Spectrum [M+H]⁺=632; ¹H NMR Spectrum (DMSO-d6) 1.44 (s, 9H), 1.94-2.01 (m, 2H), 2.88 (dd, 1H), 3.03 (dd, 1H), 3.64 (s, 3H), 3.80 (s, 3H), 3.94 (t, 2H), 4.00 (s, 2H), 4.65 (ddd, 1H), 6.75 (dd, 1H), 6.78 (d, 2H), 7.14 (d, 1H), 7.16 (d, 2H), 7.41 (dd, 1H), 7.43-7.47 (m, 2H), 8.18 (d, 1H), 9.18 (d, 1H).

### Example 34

### N-[(2,6-dichlorophenyl)carbonyl]-O-{2-[5-methoxy-6-(methylamino)pyridin-2-yl]ethyl}-L-tyrosine

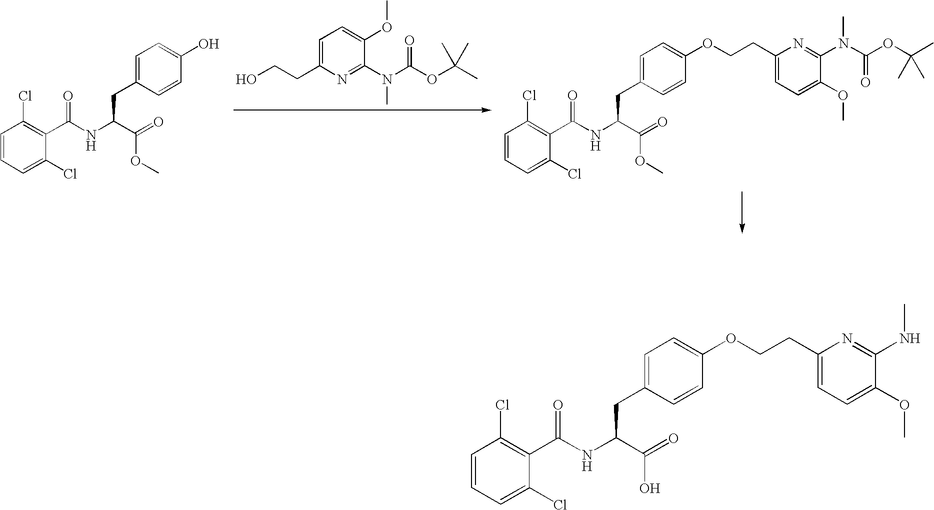

To a solution of methyl O-(2-{6-[(tert-butoxycarbonyl)(methyl)amino]-5-methoxypyridin-2-yl}ethyl)-N-[(2,6-dichlorophenyl)carbonyl]-L-tyrosinate (45 mg, 0.07 mmol) in DCM (1 ml) was added TFA (1 ml). The reaction mixture was stirred at room temperature for 2 hours. Addition of NH₃ 7M in MeOH until neutral pH was followed by filtration of the salts and evaporation to dryness. The residue was dissolved in DMF/H₂O (1 ml/0.3 ml) and LiOH (30 mg) was added. After 3 hours at room temperature, the reaction mixture was filtered and purified by C18 reverse phase chromatography (basic conditions) to afford the title compound as a white solid (20 mg, 54%); Mass Spectrum [M+H]⁺=518; ¹H NMR Spectrum (DMSO-d6) 2.81 (d, 3H), 2.86-2.94 (m, 3H), 3.05 (dd, 1H), 3.74 (s, 3H), 4.21 (t, 2H), 4.44 (dd, 1H), 5.94 (q, 1H), 6.38 (d, 1H), 6.80 (d, 2H), 6.87 (d, 1H), 7.16 (d, 2H), 7.37 (dd, 1H), 7.40-7.45 (m, 2H), 8.51 (bs, 1H).

The starting material methyl O-(2-{6-[(tert-butoxycarbonyl)(methyl)amino]-5-methoxypyridin-2-yl}ethyl)-N-[(2,6-dichlorophenyl)carbonyl]-L-tyrosinate was prepared as follows:

tert-butyl [6-(2-hydroxyethyl)-3-methoxypyridin-2-yl]methylcarbamate (68 mg, 0.24 mmol) and triphenylphosphine (121 mg, 0.46 mmol) were added to a solution of methyl N-(2,6-dichlorobenzoyl)-L-tyrosinate (80 mg, 0.22 mmol) in DCM (2 ml). ADDP (116 mg, 0.46 mmol) was then added in one portion. After 18 hours, solvent was evaporated and the residue was purified by silica gel flash chromatography (0 to 30% ethyl acetate in petroleum ether) to afford methyl O-(2-{6-[(tert-butoxycarbonyl)(methyl)amino]-5-methoxypyridin-2-yl}ethyl)-N-[(2,6-dichlorophenyl)carbonyl]-L-tyrosinate as a white solid (80 mg, 58%); Mass Spectrum [M+H]⁺=632; ¹H NMR Spectrum (DMSO-d6) 1.30 (s, 9H), 2.87 (dd, 1H), 3.04 (dd, 1H), 3.05 (t, 2H), 3.07 (s, 3H), 3.64 (s, 3H), 3.80 (s, 3H), 4.24 (t, 2H), 4.66 (ddd, 1H), 6.83 (d, 2H), 7.17 (d, 2H), 7.23 (d, 1H), 7.40 (dd, 1H), 7.43 (d, 1H), 7.43-7.47 (m, 2H), 9.17 (d, 1H)

The starting material tert-butyl [6-(2-hydroxyethyl)-3-methoxypyridin-2-yl]methylcarbamate was prepared according to the following scheme:

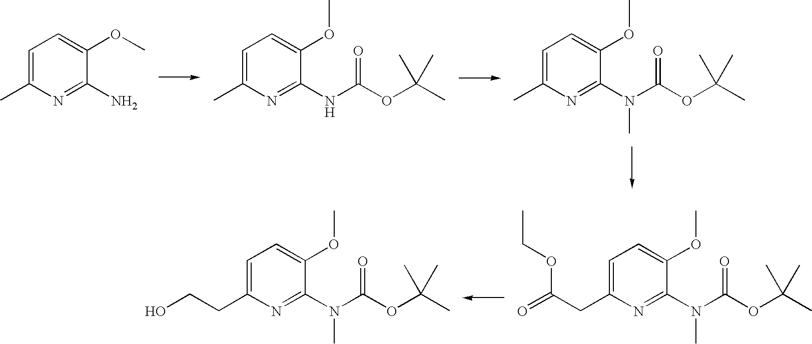

To a solution of 11.0M LiHMDS (13.2 ml, 13.2 mmol) in THF (25 ml) at −5° C. was slowly added dropwise a solution of 2-amino-3-methoxy-5-methylpyridine (1.83 g, 13.2 mmol) in THF (5 ml). After 15 minutes at −5° C., a solution of BOC₂O (3.03 g, 13.9 mmol) in THF (20 ml) was slowly added dropwise. The reaction mixture was then stirred at 0° C. for 2 hours until completion. The reaction mixture was quenched with HCl 1N (25 ml) and partially concentrated, then the aqueous layer was extracted 3 times with ethyl acetate. The organic layer was dried, concentrated and purified by silica gel flash chromatography (20% ethyl acetate in petroleum ether) to give tert-butyl (3-methoxy-6-methylpyridin-2-yl)carbamate as a pale yellow solid (0.56 g, 18%); ¹H NMR Spectrum (DMSO-d6) 1.41 (s, 9H), 2.32 (s, 3H), 3.75 (s, 3H), 7.00 (d, 1H), 7.30 (d, 1H), 8.60 (s, 1H)

To a suspension of sodium hydride (60%, 188 mg, 4.7 mmol) in acetonitrile (15 ml) was slowly added dropwise over 20 minutes tert-butyl (3-methoxy-6-methylpyridin-2-yl)carbamate (800 mg, 3.36 mmol) in acetonitrile (25 ml) at room temperature. After 1 hour, methyl iodide (0.29 ml, 4.7 mmol) in acetonitrile (25 ml) was added. After 1 hour, water was added, then acetonitrile was removed under vacuum and the aqueous layer was extracted twice with diethyl ether. The organic layer was dried, concentrated and purified by silica gel flash chromatography (0 to 30% ethyl acetate in petroleum ether) to give tert-butyl (3-methoxy-6-methylpyridin-2-yl)methylcarbamate as a pale yellow oil (650 mg, 76%); ¹H NMR Spectrum (DMSO-d6) 1.31 (s, 9H), 2.35 (s, 3H), 3.04 (s, 3H), 3.78 (s, 3H), 7.12 (d, 1H), 7.38 (d, 1H).

A solution of LDA was prepared by adding BuLi 1.6M in hexane (1.93 ml, 3.09 mmol) to diisopropylamine (0.43 ml, 3.09 mmol) in THF (40 ml) at 0° C. and stirring 45 minutes at 0° C. Then the mixture was cooled to −78° C. and slowly added dropwise to a solution of tert-butyl (3-methoxy-6-methylpyridin-2-yl)methylcarbamate (650 mg, 2.58 mmol) in THF (20 ml). After 1.5 hours at −78° C., diethylcarbonate (0.31 ml, 2.58 mmol) in THF (1 ml) was quickly added. After 1 hour, the reaction mixture was quenched at −78° C. with aqueous saturated ammonium chloride (5 ml). After addition of ethyl acetate and water, the aqueous layer was extracted twice with ethyl acetate. The organic layer was dried, concentrated and purified by silica gel flash chromatography (0 to 30% ethyl acetate in petroleum ether) to give ethyl {6-[(tert-butoxycarbonyl)(methyl)amino]-5-methoxypyridin-2-yl}acetate as a colourless oil (350 mg, 36%); ¹H NMR Spectrum (DMSO-d6) 1.17 (t, 3H), 1.31 (s, 9H), 3.04 (s, 3H), 3.72 (s, 2H), 3.81 (s, 3H), 4.08 (q, 2H), 7.23 (d, 1H), 7.46 (d, 1H).

A solution of 2N LiBH₄ in THF (0.27 ml, 0.54 mmol) was added to a stirred solution of ethyl {6-[(tert-butoxycarbonyl)(methyl)amino]-5-methoxypyridin-2-yl}acetate (250 mg, 0.77 mmol) in THF (3 ml) under nitrogen. The reaction was then heated to 60° C. overnight, then cooled down to 0° C. and carefully quenched with water. The mixture was extracted twice with ethyl acetate. The organic layer was dried, concentrated and purified by C18 reverse phase chromatography (acidic conditions) to afford tert-butyl [6-(2-hydroxyethyl)-3-methoxypyridin-2-yl]methylcarbamate (80 mg, 37%); ¹H NMR Spectrum (DMSO-d6) 1.32 (s, 9H), 2.77 (t, 2H), 3.05 (s, 3H), 3.67 (t, 2H), 3.78 (s, 3H), 4.60 (bs, 1H), 7.15 (d, 1H), 7.39 (d, 1H).

### Example 35

### N-[(2,6-dichlorophenyl)carbonyl]-O-[2-(4-methyl-5,6,7,8-tetrahydro-1,8-naphthyridin-2-yl)ethyl]-L-tyrosine

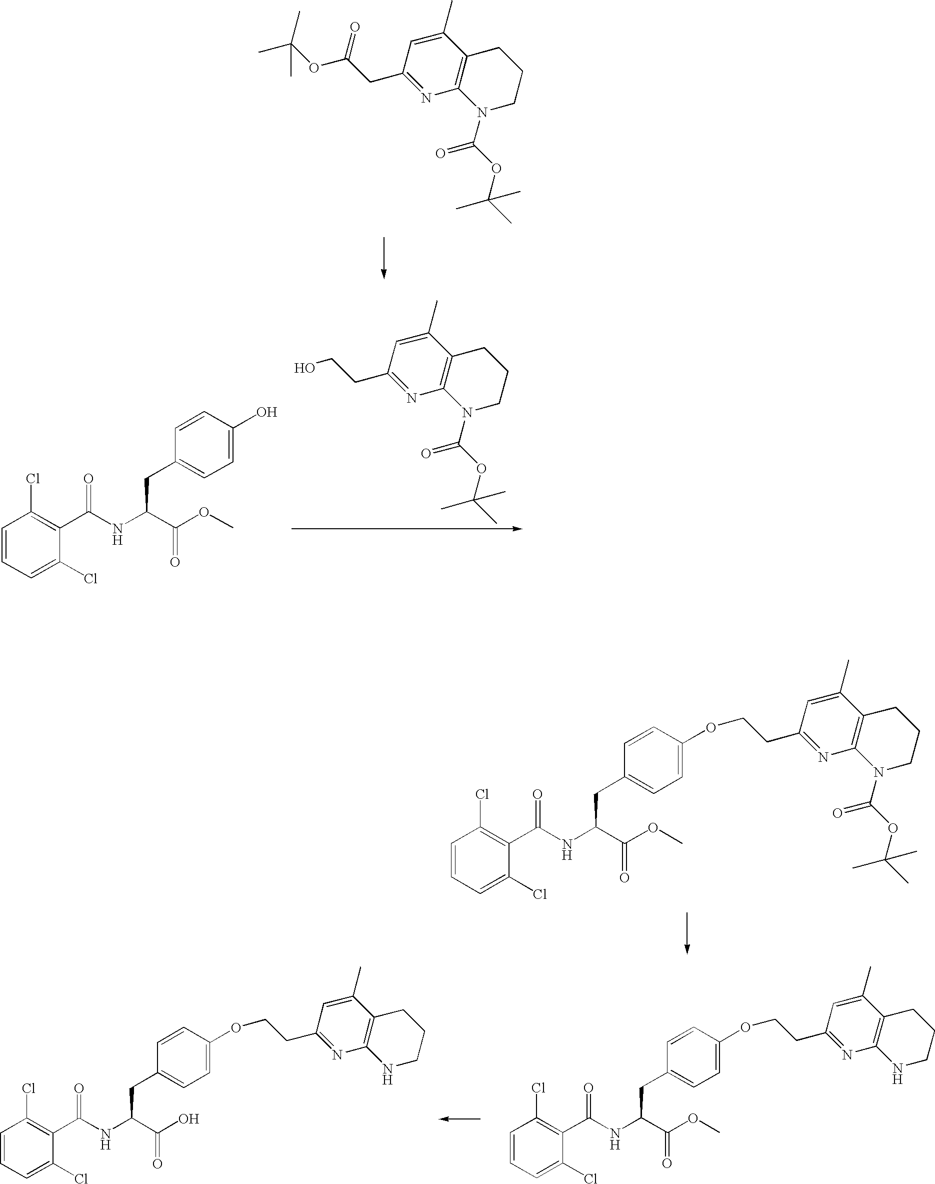

To a solution of methyl N-[(2,6-dichlorophenyl)carbonyl]-O-[2-(4-methyl-5,6,7,8-tetrahydro-1,8-naphthyridin-2-yl)ethyl]-L-tyrosinate (90 mg, 0.17 mmol) in DMA/MeOH (0.6 ml/0.1 ml) were added LiOH (50 mg) and water (0.1 ml). After 4 hours at room temperature, the reaction mixture was filtered and purified by C18 reverse phase chromatography (basic conditions) to afford the title compound as a white solid (45 mg, 51%); Mass Spectrum [M+H]⁺=528; ¹H NMR Spectrum (DMSO-d6) 1.74-1.82 (m, 2H), 2.15 (s, 3H), 2.63 (t, 2H), 2.82 (s, 2H), 2.87 (dd, 1H), 3.05 (dd, 1H), 3.20 (bs, 2H), 4.08 (t, 2H), 4.54 (dd, 1H), 6.24-6.29 (m, 2H), 6.82 (d, 2H), 7.18 (d, 2H), 7.38 (dd, 1H), 7.41-7.45 (m, 2H), 8.84 (bs, 1H)

The starting material methyl N-[(2,6-dichlorophenyl)carbonyl]-O-[2-(4-methyl-5,6,7,8-tetrahydro-1,8-naphthyridin-2-yl)ethyl]-L-tyrosinate was prepared as follows:

A solution of 2N LiBH4 in THF (1.45 ml, 2.9 mmol) was added to a stirred solution of tert-butyl 7-(2-tert-butoxy-2-oxoethyl)-5-methyl-3,4-dihydro-1,8-naphthyridine-1(2H)-carboxylate (350 mg, 0.97 mmol) described in WO 2004058761 page 89 in THF (3 ml) under nitrogen. The reaction was then heated to 65° C. overnight, then cooled down to 0° C. and carefully quenched with water. The mixture was concentrated and purified by silica gel flash chromatography (40 to 100% ethyl acetate in petroleum ether) to afford tert-butyl 7-(2-hydroxyethyl)-5-methyl-3,4-dihydro-1,8-naphthyridine-1(2H)-carboxylate as a white solid (120 mg, 43%); Mass Spectrum [M+H]⁺=293; ¹H NMR Spectrum (CDCl3) 1.51 (s, 9H), 1.64 (bs, 1H), 1.89-1.96 (m, 2H), 2.46 (s, 3H), 2.71 (t, 2H), 2.81 (t, 2H), 3.73 (t, 2H), 3.83-3.91 (m, 2H), 6.76 (s, 1H)

tert-butyl 7-(2-hydroxyethyl)-5-methyl-3,4-dihydro-1,8-naphthyridine-1(2H)-carboxylate (117 mg, 0.40 mmol) and triphenylphosphine (210 mg, 0.80 mmol) were added to a solution of methyl N-(2,6-dichlorobenzoyl)-L-tyrosinate (140 mg, 0.38 mmol) in DCM (3 ml). ADDP (202 mg, 0.80 mmol) was then added portionwise over 5 minutes. After 2 hours, solvent was evaporated and the residue was purified by silica gel flash chromatography (20 to 100% ethyl acetate in petroleum ether) to afford tert-butyl 7-(2-{4-[(2S)-2-{[(2,6-dichlorophenyl)carbonyl]amino}-3-methoxy-3-oxopropyl]phenoxy}ethyl)-5-methyl-3,4-dihydro-1,8-naphthyridine-1(2H)-carboxylate as a white foam (150 mg, 61%); Mass Spectrum [M+H]⁺=642; ¹H NMR Spectrum (DMSO-d6) 1.42 (s, 9H), 1.79-1.88 (m, 2H), 2.33 (s, 3H), 2.71 (t, 2H), 2.88 (dd, 1H), 2.96 (t, 2H), 3.04 (dd, 1H), 3.60 (t, 2H), 3.64 (s, 3H), 4.18 (t, 2H), 4.66 (ddd, 1H), 6.86 (d, 2H), 6.89 (s, 1H), 7.18 (d, 2H), 7.40 (dd, 1H), 7.43-7.48 (m, 2H), 9.18 (d, 1H)

To a solution of tert-butyl 7-(2-{4-[(2S)-2-{[(2,6-dichlorophenyl)carbonyl]amino}-3-methoxy-3-oxopropyl]phenoxy}ethyl)-5-methyl-3,4-dihydro-1,8-naphthyridine-1(2H)-carboxylate (150 mg, 0.23 mmol) in DCM (1 ml) was added TFA (3 ml). The reaction mixture was stirred at room temperature for 3 hours. Addition of NH₃ 7M in MeOH until neutral pH was followed by filtration of the salts and evaporation to dryness. The residue was purified by silica gel flash chromatography (80 to 100% ethyl acetate in DCM) to afford methyl N-[(2,6-dichlorophenyl)carbonyl]-O-[2-(4-methyl-5,6,7,8-tetrahydro-1,8-naphthyridin-2-yl)ethyl]-L-tyrosinate as a white foam (93 mg, 73%); Mass Spectrum [M+H]⁺=542; ¹H NMR Spectrum (DMSO-d6)) 1.73-1.82 (m, 2H), 2.15 (s, 3H), 2.63 (t, 2H), 2.83 (t, 2H), 2.88 (dd, 1H), 3.05 (dd, 1H), 3.18-3.23 (m, 2H), 3.64 (s, 3H), 4.09 (t, 2H), 4.66 (ddd, 1H), 6.21 (bs, 1H), 6.27 (s, 1H), 6.84 (d, 2H), 7.18 (d, 2H), 7.40 (dd, 1H), 7.43-7.47 (m, 2H), 9.18 (d, 1H).

### Example 36

### N-[(2,6-dichlorophenyl)carbonyl]-O-[2-(7-methyl-5,6,7,8-tetrahydro-1,8-naphthyridin-2-yl)ethyl]-L-tyrosine

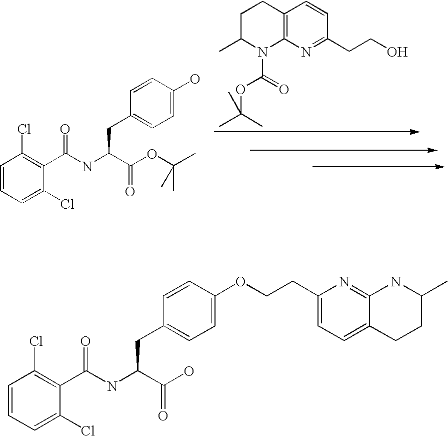

To a solution of tert-butyl 7-(2-hydroxyethyl)-2-methyl-3,4-dihydro-1,8-naphthyridine-1(2H)-carboxylate (219 mg, 0.75 mmol), triphenylphosphine (267 mg, 1.02 mmol) and tert-butyl N-(2,6-dichlorobenzoyl)-L-tyrosinate (280 mg, 0.68 mmol) described in WO 2002/018320 page 40 in DCM (2 ml) at −20° C. was slowly added dropwise a solution of ADDP (257 mg, 1.02 mmol) in DCM (2 ml). The reaction mixture was allowed to warm up to room temperature and to stir overnight. Then salts were filtered off and washed with DCM, and the solution was purified by silica gel flash chromatography (20 to 50% ethyl acetate in petroleum ether) to afford a white foam (334 mg). This foam was dissolved in DCM/TFA (2 ml/2 ml) and stirred overnight. The mixture was concentrated, then dissolved in DCM. The organic layer was washed with 1M aqueous NaOH, then brine. After evaporation, the residue was purified by C18 reverse phase chromatography (basic conditions) to afford the title compound as a white solid (50 mg, 14%); Mass Spectrum [M+H]⁺=528; ¹H NMR Spectrum (DMSO-d6) 1.14 (d, 3H), 1.34-1.44 (m, 1H), 1.78-1.87 (m, 1H), 2.55-2.69 (m, 2H), 2.82-2.91 (m, 3H), 3.04 (dd, 1H), 3.40-3.46 (m, 1H), 4.19 (t, 2H), 4.56 (ddd, 1H), 6.32 (s, 1H), 6.37 (d, 1H), 6.80 (d, 2H), 7.08 (d, 1H), 7.18 (d, 2H), 7.38 (dd, 1H), 7.41-7.46 (m, 2H), 8.89 (bs, 1H).

The starting material tert-butyl 7-(2-hydroxyethyl)-2-methyl-3,4-dihydro-1, 8-naphthyridine-1(2H)-carboxylate was prepared as follows:

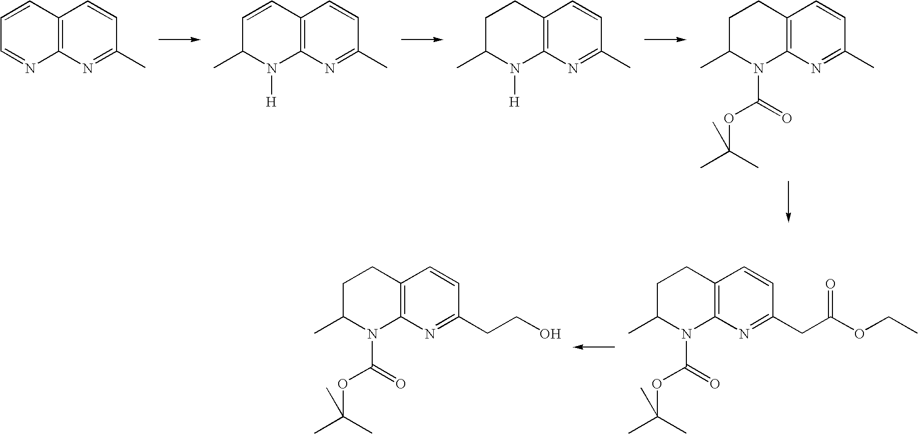

To a stirred solution of 2-methylnaphthyridine (2.0 g, 13.9 mmol) in THF (5 ml) was added at −78° C. MeLi 1.6M (7.2 ml, 41.7 mmol) over 5 minutes. The reaction mixture was left to stir at −78° C. for 2 hours, then at room temperature for 2 hours. Then a careful hydrolysis at 0° C. with water was followed by extraction of the aqueous layer with diethyl ether. The organic layer was dried and concentrated to give 2,7-dimethyl-1,2-dihydro-1,8-naphthyridine as a red oil (1.98 g, 90%); Mass Spectrum [M+H]⁺=161; ¹H NMR Spectrum (DMSO-d6) 1.20 (d, 3H), 2.14 (s, 3H), 4.41-4.49 (m, 1H), 5.46 (ddd, 1H), 6.17 (dd, 1H), 6.20 (d, 1H), 6.44 (s, 1H), 6.90 (d, 1H).

A solution of 2,7-dimethyl-1,2-dihydro-1,8-naphthyridine (1.98 g, 12.5 mmol) in ethanol (50 ml) was hydrogenated under 1.3 bars of hydrogen in the presence of Pd/C (400 mg) overnight at room temperature. The catalyst was then filtered off and the resulting mixture evaporated. The residue was purified by silica gel flash chromatography (3% NH₃/MeOH 7N in dichloromethane) to afford 2,7-dimethyl-1,2,3,4-tetrahydro-1,8-naphthyridine as a white solid (1.89 g, 93%); Mass Spectrum [M+H]⁺=163; ¹H NMR Spectrum (DMSO-d6) 1.13 (d, 3H), 1.33-1.43 (m, 1H), 1.78-1.86 (m, 1H), 2.17 (s, 3H), 2.53-2.68 (m, 2H), 3.36-3.44 (m, 1H), 6.22 (s, 1H), 6.26 (d, 1H), 7.02 (d, 1H).

2,7-dimethyl-1,2,3,4-tetrahydro-1,8-naphthyridine (1.8 g, 11.1 mmol) and an excess of BOC₂O (10 g, 45.8 mmol) were heated at 60° C. for 16 hours. Then the reaction mixture was purified by silica gel flash chromatography (0 to 50% dichloromethane in petroleum ether) to afford tert-butyl 2,7-dimethyl-3,4-dihydro-1,8-naphthyridine-1(2H)-carboxylate as a colourless oil (2.27 g, 78%); ¹H NMR Spectrum (DMSO-d6) 1.08 (d, 3H), 1.42 (s, 9H), 1.48-1.58 (m, 1H), 1.98-2.10 (m, 1H), 2.35 (s, 3H), 2.53-2.70 (m, 2H), 4.36-4.46 (m, 1H), 6.88 (d, 1H), 7.39 (d, 1H).

A solution of LDA was prepared by adding BuLi 1.6M in hexane (1.5 ml, 3.74 mmol) to diisopropylamine (0.52 ml, 3.74 mmol) in THF (5 ml) at −20° C. and stirring 60 minutes at −20° C. Then the mixture was cooled to −60° C., and slowly added dropwise to a solution of tert-butyl 2,7-dimethyl-3,4-dihydro-1,8-naphthyridine-1(2H)-carboxylate (350 mg, 1.33 mmol) and diethylcarbonate (0.60 ml, 4.94 mmol) in THF (4 ml). After 1.5 hours at −60° C., the reaction mixture was quenched at −78° C. with aqueous saturated ammonium chloride (5 ml). After addition of ethyl acetate and water, the aqueous layer was extracted twice with ethyl acetate. The organic layer was dried, concentrated and purified by silica gel flash chromatography (15 to 25% ethyl acetate in petroleum ether) to give tert-butyl 7-(2-ethoxy-2-oxoethyl)-2-methyl-3,4-dihydro-1,8-naphthyridine-1(2H)-carboxylate as a pale yellow oil (378 mg, 85%); ¹H NMR Spectrum (CDCl3) 1.19 (d, 3H), 1.25 (t, 3H), 1.49 (s, 9H), 1.55-1.64 (m partially hidden by H₂O, 1H), 2.11-2.20 (m, 1H), 2.61-2.75 (m, 2H), 3.77 (s, 2H), 4.15 (dd, 1H), 4.18 (dd, 1H), 4.57-4.66 (m, 1H), 6.99 (d, 1H), 7.36 (d, 1H).

A solution of 2N LiBH4 in THF (0.66 ml, 1.33 mmol) was added to a stirred solution of tert-butyl 7-(2-ethoxy-2-oxoethyl)-2-methyl-3,4-dihydro-1,8-naphthyridine-1(2H)-carboxylate (370 mg, 1.11 mmol) in THF (8 ml) under nitrogen. The reaction mixture was stirred at room temperature for 6 hours, then quenched with water, followed by addition of solid MgSO₄. The mixture was filtered through celite, washed with DCM, concentrated and purified by silica gel flash chromatography (30 to 50% ethyl acetate in petroleum ether) to afford tert-butyl 7-(2-hydroxyethyl)-2-methyl-3,4-dihydro-1,8-naphthyridine-1(2H)-carboxylate as a colourless oil (270 mg, 84%); ¹H NMR Spectrum (CDCl3) 1.20 (d, 3H), 1.53 (s, 9H), 1.62-1.71 (m partially hidden by H₂O, 1H), 2.61-2.79 (m, 2H), 2.85-2.98 (m, 2H), 3.88-3.97 (m, 1H), 3.98-4.07 (m, 1H), 4.59-4.67 (m, 1H), 5.50 (bs, 1H), 6.77 (d, 1H), 7.32 (d, 1H).

### Example 37.1

### N-[(2-chlorophenyl)carbonyl]-4-[3-(3,4-dihydro-2H-pyrido[3,2-b][1,4]oxazin-6-yl)propyl]-L-phenylalanine

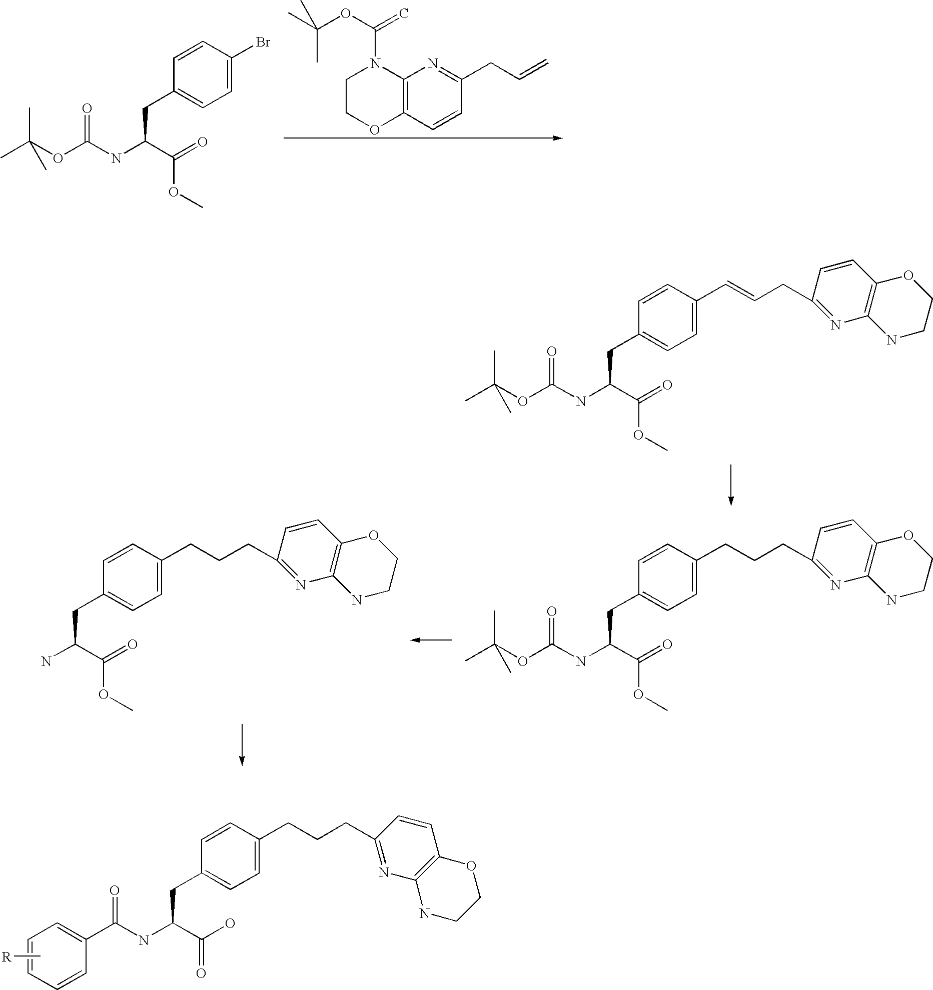

To 2-chlorobenzoic acid (37 mg, 0.24 mmol) and TBTU (87 mg, 0.23 mmol) was added a solution of methyl 4-[3-(3,4-dihydro-2H-pyrido[3,2-b][1,4]oxazin-6-yl)propyl]-L-phenylalaninate (70 mg, 0.21 mmol) in DMF (1 ml). The reaction mixture was stirred at room temperature for 72 hours. Then NaOH 6N (110 A10.64 mmol) was added, and the mixture was stirred for 1.5 hours. A filtration was directly followed by a purification by C18 reverse phase chromatography (basic conditions) to afford the title compound N-[(2-chlorophenyl)carbonyl]-4-[3-(3,4-dihydro-2H-pyrido[3,2-b][1,4]oxazin-6-yl)propyl]-L-phenylalanine as a solid.

The procedure described above for example 37.1 was repeated using the appropriate benzoic acid. Thus were obtained the compounds shown in Table 8:

TABLE 8

| | | | | | ¹H NMR Data (500 MHz) |
|---|---|---|---|---|---|
| Eg | R | Name | Yield | Mass Ion | (DMSOd6) |
| 37.1 | 2- | N-[(2- | 69% | 480 [M + H]⁺ | 1.79-1.91 (m, 2H), |
| | chloro | chlorophenyl)carbonyl]- | | | 2.45 (t, 2H), 2.54 (t, 2H), 2.95 (dd, |
| | | 4-[3- | | | 1H), 3.18 (dd, 1H), |
| | | (3,4-dihydro- | | | 3.33-3.40 (m, 2H), 4.03-4.10 (m, 2H), |
| | | 2H-pyrido[3,2- | | | 4.50 (ddd, 1H), 6.29 (d, 1H), |
| | | b][1,4]oxazin-6- | | | 6.62 (bs, 1H), 6.82 (d, 1H), |
| | | yl)propyl]-L- | | | 7.09 (d, 2H), 7.19 (d, 2H), |
| | | phenylalanine | | | 7.26 (dd, 1H), 7.34 (ddd, 1H), |
| | | | | | 7.41 (ddd, 1H), 7.44 (d, 1H), |
| | | | | | 8.36 (d, 1H) |
| 37.2 | 2- | N-[(2-chloro-6- | 93% | 498 [M + H]⁺ | 1.79-1.91 (m, 2H), 2.44 (t, |
| | chloro- | fluorophenyl)carbonyl]- | | | 2H), 2.54 (t, 2H), 2.92 (dd, |
| | 6- | 4-[3- | | | 1H), 2.12 (dd, 1H), |
| | fluoro | (3,4-dihydro- | | | 3.33-3.39 (m, 2H), 4.06 (t, 2H), |
| | | 2H-pyrido[3,2- | | | 4.48 (ddd, 1H), 6.30 (d, 1H), |
| | | b][1,4]oxazin-6- | | | 6.60 (s, 1H), 6.82 (d, 1H), 7.06 (d, |
| | | yl)propyl]-L- | | | 2H), 7.18 (d, 2H), 7.24 (dd, |
| | | phenylalanine | | | 1H), 7.30 (d, 1H), 7.43 (dd, |
| | | | | | 1H), 8.60 (d, 1H) |
| 37.3 | 2- | N-[(2-chloro-4- | 76% | 498 [M + H]⁺ | 1.79-1.90 (m, 2H), 2.43 (t, |
| | chloro- | fluorophenyl)carbonyl]- | | | 2H), 2.53 (t, 2H), 2.92 (dd, |
| | 4- | 4-[3- | | | 1H), 3.16 (dd, 1H), |
| | fluoro | (3,4-dihydro- | | | 3.32-3.39 (m, 2H), 4.03-4.09 (m, 2H), |
| | | 2H-pyrido[3,2- | | | 4.47 (ddd, 1H), 6.28 (d, 1H), |
| | | b][1,4]oxazin-6- | | | 6.59 (bs, 1H), 6.81 (d, 1H), |
| | | yl)propyl]-L- | | | 7.08 (d, 2H), 7.17 (d, 2H), |
| | | phenylalanine | | | 7.22 (dd, 1H), 7.30 (dd, 1H), |
| | | | | | 7.43 (dd, 1H), 8.39 (d, 1H) |
| 37.4 | 2,6- | N-[(2,6- | 84% | 514 [M + H]⁺ | 1.77-1.89 (m, 2H), 2.44 (t, |
| | dichloro | dichlorophenyl) | | | 2H), 2.52 (t partially hidden by |
| | | carbonyl]-4-[3- | | | DMSOd5, 2H), 2.91 (dd, 1H), |
| | | (3,4-dihydro- | | | 3.11 (dd, 1H), 3.36 (bs, 2H), |
| | | 2H-pyrido[3,2- | | | 4.06 (t, 2H), 4.54 (ddd, 1H), |
| | | b][1,4]oxazin-6- | | | 6.29 (d, 1H), 6.59 (bs, 1H), |
| | | yl)propyl]-L- | | | 6.81 (d, 1H), 7.05 (d, 2H), |
| | | phenylalanine | | | 7.19 (d, 2H), 7.36 (dd, 1H), |
| | | | | | 7.39-7.44 (m, 2H), 8.64 (d, 1H) |

The starting methyl 4-[3-(3,4-dihydro-2H-pyrido[3,2-b][1,4]oxazin-6-yl)propyl]-L-phenylalaninate used in Example 37 was prepared as follows.

A solution of tert-butyl 6-prop-2-en-1-yl-2,3-dihydro-4H-pyrido[3,2-b][1,4]oxazine-4-carboxylate (1.05 g, 3.8 mmol), methyl 4-bromo-N-(tert-butoxycarbonyl)-L-phenylalaninate (1.36 g, 3.8 mmol), tri-O-tolylphosphine (231 mg, 0.76 mmol), Palladium(II) acetate (85 mg, 0.38 mmol) and DIPEA (0.66 ml, 3.8 mmol) in acetonitrile (4 ml) was degassed, then sealed and heated to 140° C. for 25 minutes in microwave. After concentration, a purification by silica gel flash chromatography (10 to 60% ethyl acetate in petroleum ether) afforded methyl N-(tert-butoxycarbonyl)-4-[3-(3,4-dihydro-2H-pyrido[3,2-b][1,4]oxazin-6-yl)prop-1-en-1-yl]-L-phenylalaninate as a cis-trans mixture (1.5 g, 71%); Mass Spectrum [M+H]⁺=454.

A mixture of methyl N-(tert-butoxycarbonyl)-4-[3-(3,4-dihydro-2H-pyrido[3,2-b][1,4]oxazin-6-yl)prop-1-en-1-yl]-L-phenylalaninate (1.3 g, 2.9 mmol) and Pd/C 10% (300 mg) in ethanol (60 ml) was hydrogenated under 4 bar of H₂ for 4 hours, filtered through celite and purified by silica gel flash chromatography (60 to 70% ethyl acetate in petroleum ether) to afford methyl N-(tert-butoxycarbonyl)-4-[3-(3,4-dihydro-2H-pyrido[3,2-b][1,4]oxazin-6-yl)propyl]-L-phenylalaninate as a beige foam (866 mg, 66%); Mass Spectrum [M+H]⁺=456; ¹H NMR Spectrum (CDCl3) 1.41 (s, 9H), 1.90-2.00 (m, 2H), 2.58 (t, 2H), 2.62 (t, 2H), 2.96-3.10 (m, 2H), 3.51-3.57 (m, 2H), 3.71 (s, 3H), 4.18-4.23 (m, 2H), 4.52-4.60 (m, 1H), 4.91-5.01 (m, 2H), 6.39 (d, 1H), 6.89 (d, 1H), 7.02 (d, 2H), 7.11 (d, 2H).

Methyl N-(tert-butoxycarbonyl)-4-[3-(3,4-dihydro-2H-pyrido[3,2-b][1,4]oxazin-6-yl)propyl]-L-phenylalaninate (866 mg, 1.9 mmol) in DCM (1 ml) was stirred with TFA (5 ml) 1.5 hour at room temperature. Evaporation was followed by dissolution in DCM and slow addition of a solution of NH₃ 7N in MeOH at 0° C. Removal of salts by filtration was followed by a purification by silica gel flash chromatography (1 to 5% NH₃/MeOH 7N in DCM) to give methyl 4-[3-(3,4-dihydro-2H-pyrido[3,2-b][1,4]oxazin-6-yl)propyl]-L-phenylalaninate as a pale yellow solid (477 mg, 70%); Mass Spectrum [M+H]⁺=356; ¹H NMR Spectrum (CDCl3) 1.78 (bs, 2H), 1.93-2.00 (m, 2H), 2.59 (t, 2H), 2.63 (t, 2H), 2.82 (dd, 1H), 3.06 (dd, 1H), 3.52-3.57 (m, 2H), 3.71 (t, 1H), 3.72 (s, 3H), 4.18-4.22 (m, 2H), 5.06 (bs, 1H), 6.39 (d, 1H), 6.90 (d, 1H), 7.09 (d, 2H), 7.13 (d, 2H).

The tert-butyl 6-prop-2-en-1-yl-2,3-dihydro-4H-pyrido[3,2-b][1,4]oxazine-4-carboxylate starting material was prepared according to the following scheme:

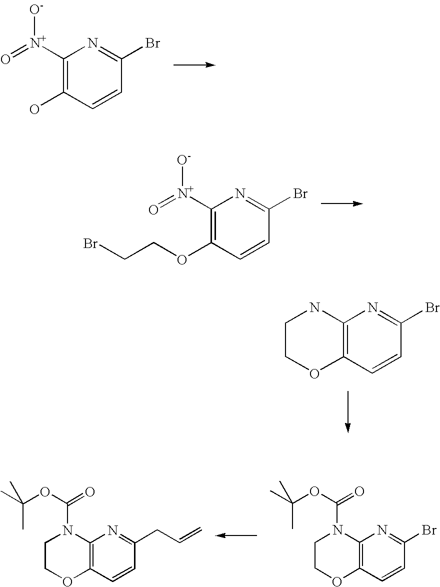

To 6-bromo-3-hydroxy-2-nitropyridine (14.6 g, 66.7 mmol) in DMF (60 ml) were added potassium carbonate (27.6 g, 200 mmol) and dibromoethane (57.5 ml, 667 mmol). The reaction mixture was heated at 80° C. for 6 hours. After filtration of salts and evaporation of DMF, the residue was purified by silica gel flash chromatography (10 to 30% ethyl acetate in petroleum ether) to give 6-bromo-3-(2-bromoethoxy)-2-nitropyridine as a yellow oil (4.5 g, 21%); ¹H NMR Spectrum (CDCl3) 3.66 (t, 2H), 4.44 (t, 2H), 7.44 (d, 1H), 7.70 (d, 1H).

To a solution of 6-bromo-3-(2-bromoethoxy)-2-nitropyridine (4.5 g, 13.8 mmol) in glacial acetic acid (23 ml) was added iron (powder, 3.08 g, 55.2 mmol) in one portion. The reaction mixture was heated to 90° C. during 5 hours, then cooled down, diluted in ethyl acetate and filtered through a silica gel plug using ethyl acetate as eluent. After evaporation, the residue was dissolved in DMF (50 ml). Potassium carbonate (5.72 g, 41.4 mmol) was added and the mixture was heated to 90° C. for 2 hours, then to 70° C. overnight. After evaporation of solvent, the residue was dissolved in DCM, salts were removed by filtration, and the crude compound was purified by silica gel flash chromatography (30 to 50% ethyl acetate in petroleum ether) to give 6-bromo-3,4-dihydro-2H-pyrido[3,2-b][1,4]oxazine as a pale yellow oil (1.64 g, 55%); Mass Spectrum [M+H]⁺=215; ¹H NMR Spectrum (CDCl3) 3.54-3.60 (m, 2H), 4.19 (t, 2H), 5.35 (bs, 1H), 6.66 (d, 1H), 6.82 (d, 1H).

To a solution of 11.0M LiHMDS (14.5 ml, 14.5 mmol) in THF (6 ml) at −30° C. was slowly added dropwise a solution of 6-bromo-3,4-dihydro-2H-pyrido[3,2-b][1,4]oxazine (1.45 g, 6.6 mmol) in THF (6 ml). After 1 hour at −30° C., a solution of BOC₂O (1.51 g, 6.9 mmol) in THF (6 ml) was slowly added dropwise. The reaction mixture was then stirred at 0° C. for 2 hours until completion. The reaction mixture was quenched with HCl 1N (25 ml) and partially concentrated, then the aqueous layer was extracted twice with ethyl acetate. The organic layer was dried, concentrated and purified by silica gel flash chromatography (10 to 20% ethyl acetate in petroleum ether) to give tert-butyl 6-bromo-2,3-dihydro-4H-pyrido[3,2-b][1,4]oxazine-4-carboxylate as a white solid (1.84 g, 88%); ¹H NMR Spectrum (CDCl3) 1.56 (s, 9H), 3.89 (t, 2H), 4.24 (t, 2H), 7.03 (d, 1H), 7.09 (d, 1H).

A solution of tert-butyl 6-bromo-2,3-dihydro-4H-pyrido[3,2-b][1,4]oxazine-4-carboxylate (1.84 g, 5.8 mmol), 2-allyl-4,4,5,5-tetramethyl-1,3,2-dioxaborolane (2.10 ml, 11.7 mmol), Pd(PPh₃)₄ (670 mg, 0.58 mmol) and cesium fluoride (2.67 g, 17.5 mmol) in acetonitrile (45 ml) was degassed, then heated to 80° C. for 3 hours. The reaction mixture was cooled down, concentrated, then diluted in ether (50 ml), washed with water and dried with MgSO₄. After concentration, a purification by silica gel flash chromatography (10 to 15% ethyl acetate in petroleum ether) afforded tert-butyl 6-prop-2-en-1-yl-2,3-dihydro-4H-pyrido[3,2-b][1,4]oxazine-4-carboxylate as a colorless oil (985 mg, 61%); ¹H NMR Spectrum (CDCl3) 1.54 (s, 9H), 3.48 (d, 2H), 3.89 (t, 2H), 4.23 (t, 2H), 5.07-5.16 (m, 2H), 6.01-6.10 (m, 1H), 6.84 (d, 1H), 7.09 (d, 1H).

### Example 38

### N-[(2,6-dichlorophenyl)carbonyl]-4-({[6-(methylamino)pyridin-2-yl]methoxy}methyl)-L-phenylalanine

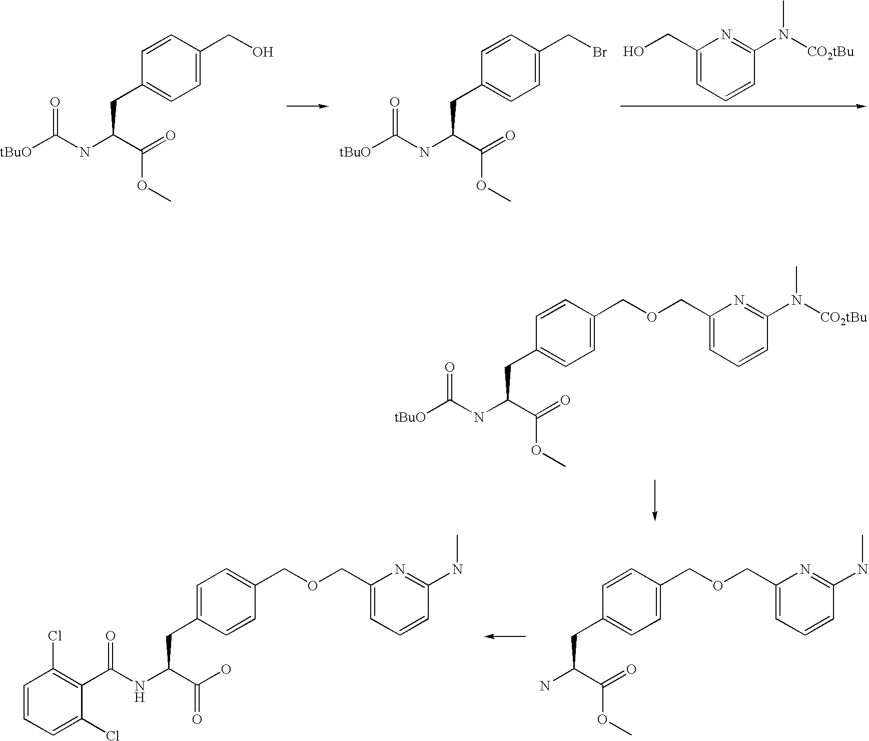

To a solution of methyl 4-({[6-(methylamino)pyridin-2-yl]methoxy}methyl)-L-phenylalaninate (17 mg, 0.05 mmol) in DCM (1 ml) were successively added at 0° C. triethylamine (11 μl, 0.08 mmol) then dichlorobenzoyl chloride (7 μl, 0.05 mmol). The reaction mixture was stirred for 2 hours at room temperature, then LiOH (22 mg, 0.52 mmol) in water (0.2 ml) was added and the mixture stirred for 2 hours. The reaction mixture was filtered and purified by C18 reverse phase chromatography (basic conditions) to afford the title compound as a white solid (10 mg, 40%); Mass Spectrum [M+H]⁺=488; ¹H NMR Spectrum (DMSO-d6) 2.73 (d, 3H), 2.94 (dd, 1H), 3.13 (dd, 1H), 4.36 (s, 2H), 4.53 (s, 2H), 4.58-4.65 (m, 1H), 6.31 (d, 1H), 6.41 (q, 1H), 6.56 (d, 1H), 7.23-7.32 (m, 4H), 7.34-7.46 (m, 4H), 8.91 (bs, 1H).

The starting material methyl 4-({[6-(methylamino)pyridin-2-yl]methoxy}methyl)-L-phenylalaninate was prepared as follows:

To a solution of methyl 4-(hydroxymethyl)-N-(tert-butoxycarbonyl)-L-phenylalaninate (Synthetic communications 1998, 28, 4279)(325 mg, 1.05 mmol) in THF (6 ml) were added triphenylphosphine (304 mg, 1.16 mmol) and CBr₄ (385 mg, 1.16 mmol). The reaction mixture was stirred at room temperature for 3 hours, then filtered, concentrated and purified by silica gel flash chromatography (0 to 50% ethyl acetate in petroleum ether) to give methyl 4-(bromomethyl)-N-(tert-butoxycarbonyl)-L-phenylalaninate as a white solid (265 mg, 68%); ¹H NMR Spectrum (DMSO-d6) 1.32 (s, 9H), 2.84 (dd, 1H), 2.99 (dd, 1H), 3.61 (s, 3H), 4.17 (ddd, 1H), 4.67 (s, 2H), 7.22 (d, 2H), 7.30 (d, 1H), 7.35 (d, 2H).

To a solution of NaH (60% in oil, 28 mg, 0.70 mmol) in DMA (2 ml) was added tert-butyl [6-(hydroxymethyl)pyridin-2-yl]methylcarbamate (167 mg, 0.70 mmol) in DMA (3 ml) at 0° C. The mixture was allowed to stir at room temperature for 15 minutes, then methyl 4-(bromomethyl)-N-(tert-butoxycarbonyl)-L-phenylalaninate (238 mg, 0.64 mmol) was added and the reaction mixture was stirred overnight. Addition of water (1 ml) was followed by evaporation of solvents under vacuum. The residue was purified by silica gel flash chromatography (0 to 20% ethyl acetate in petroleum ether) to give methyl N-(tert-butoxycarbonyl)-4-[({6-[(tert-butoxycarbonyl)(methyl)amino]pyridin-2-yl}methoxy)methyl]-L-phenylalaninate (80 mg, 24%); Mass Spectrum [M+H]⁺=530; ¹H NMR Spectrum (DMSO-d6) 1.31 (s, 9H), 1.45 (s, 9H), 2.85 (dd, 1H), 2.99 (dd, 1H), 3.26 (s, 3H), 3.61 (s, 3H), 4.16 (ddd, 1H), 4.52 (s, 2H), 4.57 (s, 2H), 7.17 (d, 1H), 7.22 (d, 2H), 7.30 (m, 3H), 7.50 (d, 1H), 7.76 (dd, 1H).

To a solution of methyl N-(tert-butoxycarbonyl)-4-[({6-[(tert-butoxycarbonyl)(methyl)amino]pyridin-2-yl}methoxy)methyl]-L-phenylalaninate (80 mg, 0.15 mmol) in DCM (1 ml) was added TFA (1 ml). The reaction mixture was stirred at room temperature overnight. Addition of NH₃ 7M in MeOH until neutral pH was followed by filtration of the salts and evaporation to dryness. The residue was purified by C18 reverse phase chromatography (basic conditions) to afford methyl 4-({[6-(methylamino)pyridin-2-yl]methoxy}methyl)-L-phenylalaninate as a white solid (20 mg, 40%); Mass Spectrum [M+H]⁺=330; ¹H NMR Spectrum (DMSO-d6+TFA) 2.99 (s, 3H), 3.06-3.21 (m, 2H), 3.71 (s, 3H), 4.30-4.40 (m, 1H), 4.62 (s, 2H), 4.65 (s, 2H), 6.89 (d, 1H), 7.01 (d, 1H), 7.26 (d, 2H), 7.39 (d, 2H), 7.92 (bs, 1H).

The starting material tert-butyl [6-(hydroxymethyl)pyridin-2-yl]methylcarbamate was prepared as follows:

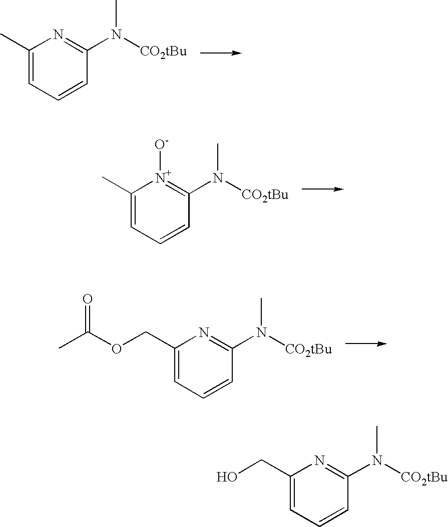

To a solution of tert-butyl methyl(6-methylpyridin-2-yl)carbamate (J. Med. Chem. 2000, 43, 22)(7.0 g, 31.5 mmol) in acetonitrile (80 ml) was added portionwise m-CPBA (6.5 g, 37.8 mmol) at room temperature. The reaction mixture was stirred overnight, then washed with an aqueous solution containing 5% of Na₂S₂O₃, then with water. The organic layer was dried, concentrated and purified by silica gel flash chromatography (50 to 100% ethyl acetate in petroleum ether) to give tert-butyl methyl(6-methyl-1-oxidopyridin-2-yl)carbamate as a white solid (3.1 g, 41%); Mass Spectrum [M+H]⁺=239; ¹H NMR Spectrum (DMSO-d6) 1.32 (s, 9H), 2.37 (s, 3H), 3.01 (s, 3H), 7.22 (dd, 1H), 7.37 (dd, 1H), 7.41 (d, 1H).

To acetic anhydride (6 ml) at 135° C. was slowly added portionwise tert-butyl methyl(6-methyl-1-oxidopyridin-2-yl)carbamate (3.06 g, 12.8 mmol). The reaction mixture was stirred 0.5 hour, then poured into an ice/water bath. The aqueous layer was extracted with a mixture of EtOAc and Et₂O. The organic layer was dried, concentrated and purified by silica gel flash chromatography (0 to 20% diethyl ether in petroleum ether) to give {6-[(tert-butoxycarbonyl)(methyl)amino]pyridin-2-yl}methyl acetate as a colorless oil (1.8 g, 50%); ¹H NMR Spectrum (DMSO-d6) 1.46 (s, 9H), 2.17 (s, 3H), 3.27 (s, 3H), 5.08 (s, 2H), 7.12 (d, 1H), 7.54 (d, 1H), 7.77 (dd, 1H).

{6-[(tert-butoxycarbonyl)(methyl)amino]pyridin-2-yl}methyl acetate (1.8 g, 6.4 mmol) in methanol (5 ml) was canulated onto a solution of potassium hydroxide (432 mg, 7.7 mmol) in methanol (25 ml) and the mixture was refluxed for 1 hour. Then solvent was removed under vacuum and water was added to the residue. The aqueous layer was extracted with Et₂O. The organic layer was dried and concentrated to give tert-butyl [6-(hydroxymethyl)pyridin-2-yl]methylcarbamate as a colorless oil (1.33 g, 87%); Mass Spectrum [M−H]⁻=237; ¹H NMR Spectrum (DMSO-d6) 1.46 (s, 9H), 3.26 (s, 3H), 4.49 (d, 2H), 5.38 (t, 1H), 7.20 (d, 1H), 7.42 (d, 1H), 7.74 (dd, 1H).

### Example 39

### N-[(2,6-dichlorophenyl)carbonyl]-4-{[2-(methylamino)quinolin-7-yl]methyl}-L-phenylalanine

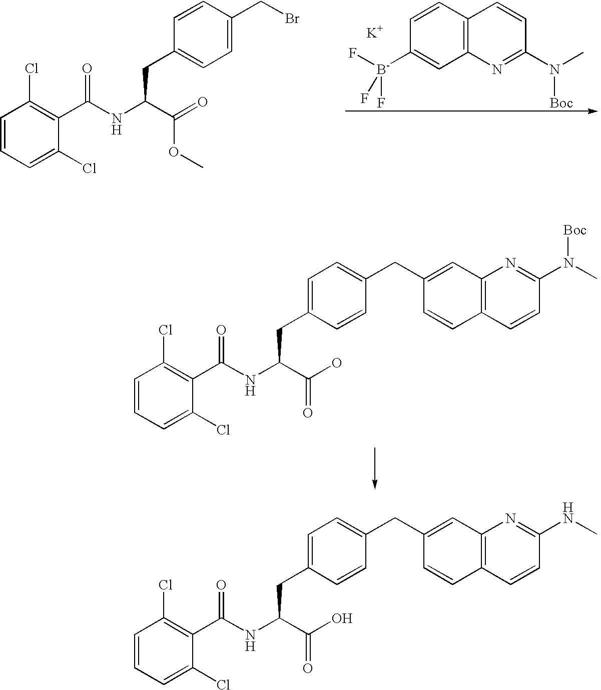

To a solution of methyl 4-({2-[(tert-butoxycarbonyl)(methyl)amino]quinolin-7-yl}methyl)-N-[(2,6-dichlorophenyl)carbonyl]-L-phenylalaninate (95 mg, 0.15 mmol) in DCM (0.6 ml) was added TFA (0.3 ml). The reaction mixture was stirred overnight. Addition of NH₃ 7M in MeOH until neutral pH was followed by filtration of the salts and evaporation to dryness. The residue was dissolved in DMF (0.5 ml) and LiOH 2M in water (0.23 ml, 0.46 mmol) was added. After 4 hours at room temperature, the crude solution was directly purified by C18 reverse phase chromatography (basic conditions) to give the title compound as a white solid (24 mg, 31%); Mass Spectrum [M+H]⁺=508 ; ¹H NMR Spectrum (DMSO-d6) 2.86 (d, 3H), 2.90 (dd, 1H) 3.09 (dd, 1H), 3.98 (s, 2H), 4.56-4.64 (m, 1H), 6.66 (d, 1H), 6.92 (q, 1H), 6.98 (dd, 1H), 7.16 (d, 2H), 7.21 (d, 2H), 7.32 (s, 1H), 7.37 (dd, 1H), 7.39-7.42 (m, 2H), 7.49 (d, 1H), 7.75 (d, 1H), 8.94 (bs, 1H)

The methyl 4-({2-[(tert-butoxycarbonyl)(methyl)amino]quinolin-7-yl}methyl)-N-[(2,6-dichlorophenyl)carbonyl]-L-phenylalaninate starting material was prepared as follows:

To a solution of methyl 4-(bromomethyl)-N-[(2,6-dichlorophenyl)carbonyl]-L-phenylalaninate (190 mg, 0.43 mmol) in THF/water (2 ml/0.2 ml) were successively added potassium {2-[(tert-butoxycarbonyl)(methyl)amino]quinolin-7-yl}(trifluoro)borate(1-) (171 mg, 0.47 mmol), cesium carbonate (417 mg, 1.28 mmol) and PdCl₂dppf (35 mg, 0.043 mmol). The resulting solution was degassed under argon, then heated at 85° C. for 3 hours in a sealed tube. Solvents were removed under vacuum, and the residue was purified by silica gel flash chromatography (30% ethyl acetate in petroleum ether) to afford methyl 4-({2-[(tert-butoxycarbonyl)(methyl)amino]quinolin-7-yl}methyl)-N-[(2,6-dichlorophenyl)carbonyl]-L-phenylalaninate as a white foam (100 mg, 38%); Mass Spectrum [M+H]⁺=622; ¹H NMR Spectrum (DMSOd6) 1.48 (s, 9H), 2.91 (dd, 1H), 3.09 (dd, 1H), 3.39 (s, 3H), 3.64 (s, 3H), 4.10 (s, 2H), 4.69 (ddd, 1H), 7.18-7.25 (m, 4H), 7.35-7.43 (m, 4H), 7.65 (s, 1H), 7.73 (d, 1H), 7.82 (d, 1H), 8.20 (d, 1H), 9.18 (d, 1H).

The {2-[(tert-butoxycarbonyl)(methyl)amino]quinolin-7-yl}(trifluoro)borate(1-) starting material was prepared according to the following scheme:

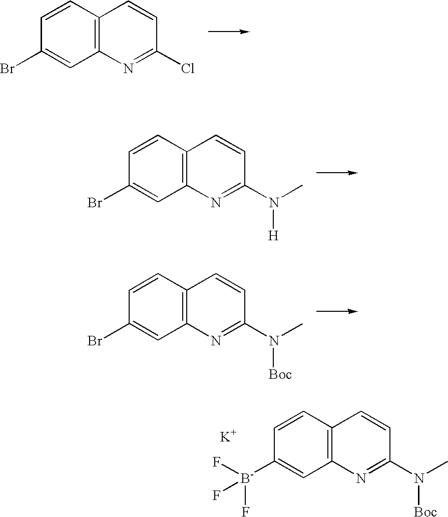

To 7-bromo-2-chloroquinoline (4.23 g, 17.4 mmol) (J; Med. Chem. 1988, 31, 2048) was added a solution of methylamine 33% in ethanol (33 ml, 86.7 mmol). The resulting suspension was heated at 130° C. for 0.5 hour under microwave. The reaction mixture was evaporated to dryness, then dissolved in DCM/MeOH and adsorbed on silica. Purification by silica gel flash chromatography (10 to 35% ethyl acetate in petroleum ether) afforded 7-bromo-N-methylquinolin-2-amine as a white powder (2.91 g, 70%); Mass Spectrum [M+H]⁺=238; ¹H NMR Spectrum (CDCl3) 3.08 (d, 3H), 4.78 (bs, 1H), 6.62 (d, 1H), 7.28 (dd, 1H), 7.42 (d, 1H), 7.75 (d, 1H), 7.88 (d, 1H)

To a solution of LiHMDS 1.0M in THF (14.7 ml, 14.7 mmol) in THF (30 ml) at 0° C. was added a solution of 7-bromo-N-methylquinolin-2-amine (2.9 g, 12.2 mmol) in THF (5 ml). The resulting dark green solution was stirred at 0° C. for 1 hour, then BOC₂O (2.79 g, 12.8 mmol) in THF (1 ml) was added and the mixture was allowed to stir 1 hour at room temperature. The reaction mixture was diluted in ethyl acetate and washed with water slightly acidified with HCl 2N, then water, aqueous saturated NaHCO₃. The organic layer was dried, concentrated and purified by silica gel flash chromatography (0 to 4% diethyl ether in petroleum ether) to give tert-butyl (7-bromoquinolin-2-yl)methylcarbamate as a beige powder (3.81 g, 92%); ¹H NMR Spectrum (DMSO-d6) 1.50 (s, 9H), 3.43 (s, 3H), 7.66 (dd, 1H), 7.88 (1, 2H), 7.90 (d, 1H), 8.04 (d, 1H), 8.30 (d, 1H)

To a solution of tert-butyl (7-bromoquinolin-2-yl)methylcarbamate (337 mg, 1.0 mmol) in DMSO (1 ml) were successively added bis(pinacolato)diboron (508 mg, 2.0 mmol), potassium acetate (294 mg, 3.0 mmol) and PdCl₂dppf (49 mg, 0.06 mmol). The resulting solution was degassed under argon, then heated at 80° C. for 1.5 hours. The reaction mixture was dissolved in DCM and washed with water. The organic layer was dried and evaporated to give a brown gum which was dissolved in acetone (1 ml). An aqueous saturated solution of KHF₂ (˜4.5M, 1.1 ml, 5 mmol) was added, resulting in the formation of a precipitate. The mixture was stirred at room temperature for 1.5 hours, then diluted with water. The precipitate was filtered, washed with water, then diethyl ether and dried to give potassium {2-[(tert-butoxycarbonyl)(methyl)amino]quinolin-7-yl}(trifluoro)borate(1-) as a brown solid (176 mg, 48%); ¹H NMR Spectrum (DMSO-d6+TFAd) 1.52 (s, 9H), 3.51 (s, 3H), 7.84 (d, 1H), 7.88 (d, 1H), 8.00 (d, 1H), 8.13 (s, 1H), 8.90 (d, 1H).

The methyl 4-(bromomethyl)-N-[(2,6-dichlorophenyl)carbonyl]-L-phenylalaninate starting material was prepared as follows:

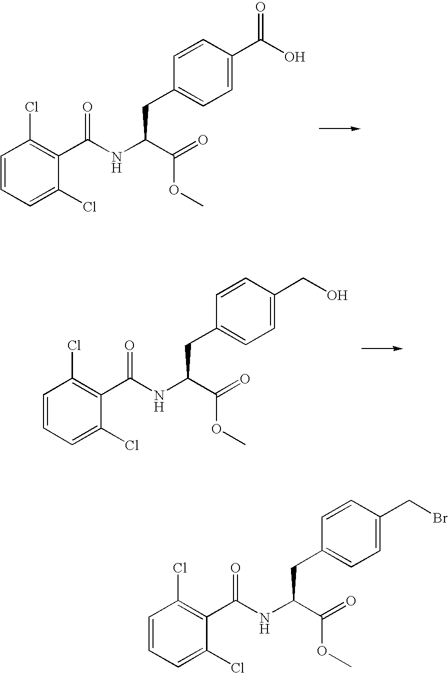

To a solution of 4-{(2S)-2-[(2,6-dichlorobenzoyl)amino]-3-methoxy-3-oxopropyl}benzoic acid (528 mg, 1.3 mmol) (example 25) in THF (15 ml) was added dropwise at 0° C. BH₃.Me₂S 2.0M in THF (3.0 ml, 6.0 mmol). After 4 hours at room temperature, the reaction had barely started, therefore BH₃ 1.0M in THF (1.0 ml, 1.0 mmol) was added. The reaction was completed in 8 hours. After careful addition of MeOH, the reaction mixture was concentrated. The residue was dissolved in DCM, washed with aqueous HCl 2N, then brine. The organic layer was dried, evaporated and purified by silica gel flash chromatography (0 to 25% ethyl acetate in DCM) to give methyl N-[(2,6-dichlorophenyl)carbonyl]-4-(hydroxymethyl)-L-phenylalaninate as a sticky white foam (400 mg, 97%); Mass Spectrum [M+H]⁺=382; ¹H NMR Spectrum (DMSO-d6) (DMSOd6+TFAd) 1.52 (s, 9H), 3.51 (s, 3H), 7.84 (d, 1H), 7.88 (d, 1H), 8.00 (d, 1H), 8.13 (s, 1H), 8.90 (d, 1H).

To a solution of methyl N-[(2,6-dichlorophenyl)carbonyl]-4-(hydroxymethyl)-L-phenylalaninate (440 mg, 1.15 mmol) in THF (8 ml) were added triphenylphosphine (333 mg, 1.27 mmol) and CBr₄ (421 mg, 1.27 mmol). After 18 hours at room temperature, a white precipitate is removed by filtration and the remaining solution is concentrated, then purified by silica gel flash chromatography (100% DCM). The residue is precipitated in a mixture of DCM and petroleum ether to provide after filtration methyl N-[(2,6-dichlorophenyl)carbonyl]-4-(bromomethyl)-L-phenylalaninate as a white solid (200 mg, 39%); Mass Spectrum [M+H]⁺=445 ; ¹H NMR Spectrum (DMSOd6) 2.93 (dd, 1H), 3.15 (dd, 1H), 3.66 (s, 3H), 4.68 (d, 2H), 4.75 (ddd, 1H), 7.27 (d, 2H), 7.36 (d, 2H), 7.39 (dd, 1H), 7.42-7.47 (m, 2H), 9.20 (d, 1H).

### Example 40

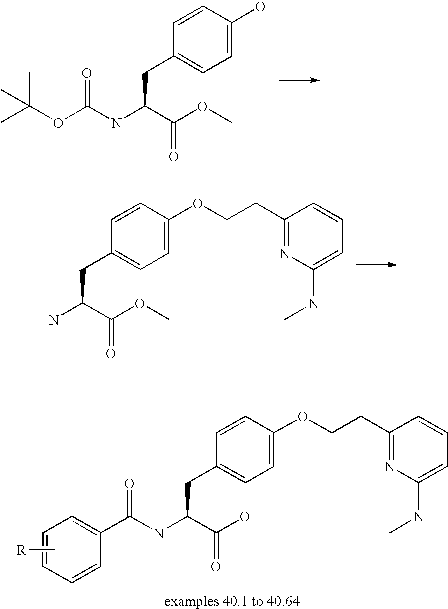

To 2,4-dichlorobenzoic acid (76 mg, 0.40 mmol) and TBTU (152 mg, 0.40 mmol) was added a solution of methyl O-{2-[6-(methylamino)pyridin-2-yl]ethyl}-L-tyrosinate (120 mg, 0.36 mmol) in DMF (1 ml). The reaction mixture was stirred at room temperature for 72 hours. Then LiOH (67 mg, 1.46 mmol) and water (0.2 ml) were added, and the mixture was stirred 24 hours. A filtration was directly followed by a purification by C18 reverse phase chromatography (basic conditions) to afford example 40.1 (N-[(2,4-dichlorophenyl)carbonyl]-O-{2-[6-(methylamino)pyridin-2-yl]ethyl}-L-tyrosine) as a solid.

The procedure described above for example 40.1 was repeated by coupling methyl O-{2-[6-(methylamino)pyridin-2-yl]ethyl}-L-tyrosinate with the appropriate benzoic acid. Thus were obtained the compounds shown in Table 9:

TABLE 9

| | | | | | ¹H NMR Data (500 MHz - |
|---|---|---|---|---|---|
| Example | R | Name | Yield | Mass Ion | DMSO d6) |
| 40.1 | 2,4- | N-[(2,4- | 58% | 488 [M + H]⁺ | 2.74 (d, 3H), 2.90 (dd, |
| | dichloro | dichlorophenyl)carbonyl]- | | | 1H), 2.93 (t, 2H), |
| | | O-{2-[6- | | | 3.13 (dd, 1H), 4.24 (t, 2H), |
| | | (methylamino)pyridin- | | | 4.33 (ddd, 1H), 6.26 (d, |
| | | 2-yl]ethyl}-L- | | | 1H), 6.36 (q, 1H), |
| | | tyrosine | | | 6.42 (d, 1H), 6.80 (d, 2H), |
| | | | | | 7.13 (d, 2H), |
| | | | | | 7.26-7.33 (m, 2H), 7.45 (dd, 1H), |
| | | | | | 7.61 (d, 1H), 8.15 (d, 1H) |
| 40.2 | 2-chloro- | N-[(2-chloro-3,6- | 52% | 490 [M + H]⁺ | 2.74 (d, 3H), 2.86 (dd, |
| | 3,6- | difluorophenyl)carbonyl]- | | | 1H), 2.94 (t, 2H), |
| | difluoro | O-{2-[6- | | | 3.07 (dd, 1H), 4.26 (t, 2H), |
| | | (methylamino)pyridin- | | | 4.56 (ddd, 1H), 6.26 (d, |
| | | 2-yl]ethyl}-L- | | | 1H), 6.36 (q, 1H), 6.43 (d, |
| | | tyrosine | | | 1H), 6.83 (d, 2H), 7.17 (d, |
| | | | | | 2H), 7.30 (dd, 1H), |
| | | | | | 7.33 (ddd, 1H), 7.51 (ddd, 1H), |
| | | | | | 9.05 (d, 1H) |
| 40.3 | 2-chloro-5- | N-[(2-chloro-5- | 61% | 468 [M + H]⁺ | 2.27 (s, 3H), 2.73 (d, |
| | methyl | methylphenyl)carbonyl]- | | | 3H), 2.89 (dd, 1H), |
| | | O-{2-[6- | | | 2.93 (t, 2H), 3.10 (dd, 1H), |
| | | (methylamino)pyridin- | | | 4.25 (t, 2H), 4.44 (ddd, |
| | | 2-yl]ethyl}-L- | | | 1H), 6.26 (d, 1H), |
| | | tyrosine | | | 6.35 (q, 1H), 6.42 (d, 1H), |
| | | | | | 6.84 (d, 2H), 7.03 (d, |
| | | | | | 1H), 7.17 (d, 2H), |
| | | | | | 7.22 (dd, 1H), 7.29 (dd, 1H), |
| | | | | | 7.31 (d, 1H), 8.39 (d, 1H) |
| 40.4 | 2,4,6- | O-{2-[6- | 34% | 462 [M + H]⁺ | 1.95 (s, 6H), 2.18 (s, 3H), |
| | trimethyl | (methylamino)pyridin- | | | 2.75 (s, 3H), 2.80 (dd, |
| | | 2-yl]ethyl}-N- | | | 1H), 2.93 (t, 2H), |
| | | [2,4,6- | | | 3.11 (dd, 1H), 4.25 (t, 2H), |
| | | trimethylphenyl)carbonyl]- | | | 4.49 (dd, 1H), 6.27 (d, |
| | | L-tyrosine | | | 1H), 6.37 (q, 1H), |
| | | | | | 6.42 (d, 1H), 6.75 (s, 2H), |
| | | | | | 6.84 (d, 2H), 7.16 (d, |
| | | | | | 2H), 7.30 (dd, 1H), |
| | | | | | 8.03 (d, 1H) |
| 40.5 | 2-chloro-6 | N-[(2-chloro-6- | 48% | 468 [M + H]⁺ | 2.08 (s, 3H), 2.75 (d, |
| | methyl | methylphenyl)carbonyl]- | | | 3H), 2.85 (dd, 1H), |
| | | O-{2-[6- | | | 2.93 (t, 2H), 3.09 (dd, 1H), |
| | | (methylamino)pyridin- | | | 4.25 (t, 2H), 4.47 (ddd, |
| | | 2-yl]ethyl}-L- | | | 1H), 6.27 (d, 1H), |
| | | tyrosine | | | 6.36 (q, 1H), 6.43 (d, 1H), |
| | | | | | 6.81 (d, 2H), 7.13 (d, |
| | | | | | 1H), 7.18 (d, 2H), |
| | | | | | 7.19-7.25 (m, 2H), 7.30 (dd, |
| | | | | | 1H), 8.36 (d, 1H) |
| 40.6 | 2-chloro-4- | N-[(2-chloro-4- | 61% | 472 [M + H]⁺ | 2.74 (d, 3H), 2.88 (dd, |
| | fluoro | fluorophenyl)carbonyl]- | | | 1H), 2.93 (t, 2H), |
| | | O-{2-[6- | | | 3.10 (dd, 1H), 4.25 (t, 2H), |
| | | (methylamino)pyridin- | | | 4.43 (ddd, 1H), 6.26 (d, |
| | | 2-yl]ethyl}-L- | | | 1H), 6.35 (q, 1H), |
| | | tyrosine | | | 6.42 (d, 1H), 6.83 (d, 2H), |
| | | | | | 7.16 (d, 2H), 7.25 (ddd, |
| | | | | | 1H), 7.28-7.34 (m, 2H), |
| | | | | | 7.45 (dd, 1H), 8.48 (d, |
| | | | | | 1H) |
| 40.7 | 2-chloro-6- | N-[2-chloro-6- | 59% | 472 [M + H]⁺ | 2.74 (d, 3H), 2.88 (dd, |
| | fluoro | fluorophenyl)carbonyl]- | | | 1H), 2.93 (t, 2H), |
| | | O-{2-[6- | | | 3.07 (dd, 1H), 4.24 (t, 2H), |
| | | (methylamino)pyridin- | | | 4.43 (ddd, 1H), 6.26 (d, |
| | | 2-yl]ethyl}-L- | | | 1H), 6.36 (q, 1H), |
| | | tyrosine | | | 6.43 (d, 1H), 6.80 (d, 2H), |
| | | | | | 7.15 (d, 2H), 7.23 (dd, |
| | | | | | 1H), 7.27-7.33 (m, 2H), |
| | | | | | 7.43 (ddd, 1H), 8.59 (d, |
| | | | | | 1H) |
| 40.8 | 2,6- | N-[(2,6- | 41% | 448 [M + H]⁺ | 1.98 (s, 6H), 2.75 (d, 3H), |
| | dimethyl | dimethylphenyl)carbonyl]- | | | 2.80 (dd, 1H), 2.93 (t, |
| | | O-{2,[6- | | | 2H), 3.11 (dd, 1H), |
| | | (methylamino)pyridin- | | | 4.25 (t, 2H), 4.53 (ddd, 1H), |
| | | 2-yl]ethyl}-L- | | | 6.27 (d, 1H), 6.36 (q, 1H), |
| | | tyrosine | | | 6.42 (d, 1H), 6.82 (d, 2H), |
| | | | | | 6.94 (d, 2H), 7.09 (dd, |
| | | | | | 1H), 7.18 (d, 2H), |
| | | | | | 7.30 (dd, 1H), 8.20 (d, 1H) |
| 40.9 | 6-chloro-2- | N-[(6-chloro-2 | 61% | 486 [M + H]⁺ | 2.20 (s, 3H), 2.74 (d, 3H), |
| | fluoro-3- | fluoro-3- | | | 2.87 (dd, 1H), 2.93 (t, |
| | methyl | methylphenyl)carbonyl]- | | | 2H), 3.05 (dd, 1H), |
| | | O-{2-[6- | | | 4.25 (t, 2H), 4.48 (ddd, 1H), |
| | | (methylamino)pyridin- | | | 6.26 (d, 1H), 6.35 (q, 1H), |
| | | 2-yl]ethyl}-L- | | | 6.43 (d, 1H), 6.82 (d, 2H), |
| | | tyrosine | | | 7.16 (d, 2H), 7.18 (d, 1H), |
| | | | | | 7.26-7.34 (m, 2H), |
| | | | | | 8.73 (bs, 1H) |
| 40.10 | 2-methyl | O-{2-[6- | 58% | 434 [M + H]⁺ | 2.17 (s, 3H), 2.74 (d, 3H), |
| | | (methylamino)pyridin- | | | 2.89 (dd, 1H), 2.93 (t, |
| | | 2-yl]ethyl}-N- | | | 2H), 3.12 (dd, 1H), |
| | | [(2- | | | 4.25 (t, 2H), 4.45 (ddd, 1H), |
| | | methylphenyl)carbonyl]- | | | 6.26 (d, 1H), 6.36 (q, 1H), |
| | | L-tyrosine | | | 6.42 (d, 1H), 6.83 (d, 2H), |
| | | | | | 7.13-7.20 (m, 5H), |
| | | | | | 7.25-7.33 (m, 2H), 8.15 (d, 1H) |
| 40.11 | 2-chloro | N-[(2- | 56% | 454 [M + H]⁺ | 2.74 (d, 3H), 2.90 (dd, |
| | | chlorophenyl)carbonyl]- | | | 1H), 2.93 (t, 2H), |
| | | O-{2-[6- | | | 3.11 (dd, 1H), 4.25 (t, 2H, |
| | | (methylamino)pyridin- | | | 4.41 (ddd, 1H), 6.26 (d, |
| | | 2-yl]ethyl}-L- | | | 1H), 6.35 (q, 1H), |
| | | tyrosine | | | 6.43(d, 1H), 6.83 (d, 2H), |
| | | | | | 7.16 (d, 2H), 7.27 (dd, |
| | | | | | 1H), 7.30 (ddd, 1H), |
| | | | | | 7.35 (ddd, 1H), 7.41 (ddd, |
| | | | | | 1H), 7.45 (dd, 1H), |
| | | | | | 8.31 (d, 1H) |
| 40.12 | 5-chloro- | N-[(5-chloro-1,3- | 70% | 498 [M + H]⁺ | 2.74 (d, 3H), |
| | 1,3- | benzodioxol-4- | | | 2.89-2.98 (m, 3H), 3.08 (dd, 1H), |
| | benzodioxol- | yl)carbonyl]-O-{2- | | | 4.18 (dd, 1H), 4.23 (t, |
| | 4-yl | [6- | | | 2H), 6.08 (d, 2H), 6.26 (d, |
| | | (methylamino)pyridin- | | | 1H), 6.35 (q, 1H), 6.43 (d, |
| | | 2-yl]ethyl}-L- | | | 1H), 6.75 (d, 2H), 6.88 (d, |
| | | tyrosine | | | 1H), 6.92 (d, 1H), 7.11 (d, |
| | | | | | 2H), 7.29 (dd, 1H), |
| | | | | | 7.73 (d, 1H) |
| 40.13 | 2-fluoro-6- | N-[(2-fluoro-6- | 63% | 452 [M + H]⁺ | 2.34 (s, 3H), 2.73 (d, |
| | methyl | methylphenyl)carbonyl]- | | | 3H), 2.93 (t, 2H), |
| | | O-{2-[6- | | | 2.97 (dd, 1H), 3.10 (dd, 1H), |
| | | (methylamino)pyridin- | | | 4.24 (t, 2H), 4.47 (ddd, |
| | | 2-yl]ethyl}-L- | | | 1H), 6.26 (d, 1H), |
| | | tyrosine | | | 6.35 (q, 1H), 6.42 (d, 1H), |
| | | | | | 6.81 (d, 2H), |
| | | | | | 7.06-7.12 (m, 2H), 7.13 (d, 2H), |
| | | | | | 7.29 (dd, 1H), 7.51 (dd, |
| | | | | | 1H), 8.09-8.15 (m, 1H) |
| 40.16 | 2,3- | N-[(2,3- | 37% | 489 [M + H]⁺ | 2.75 (d, 3H), 2.89 (dd, |
| | dichloro | dichlorophenyl)carbonyl]- | | | 1H), 2.94 (t, 2H), |
| | | O-{2-[6- | | | 3.12 (dd, 1H), 4.26 (t, 2H), |
| | | (methylamino)pyridin- | | | 4.35-4.42 (m, 1H), |
| | | 2-yl]ethyl}-L- | | | 6.27 (d, 1H), 6.36 (q, 1H), |
| | | tyrosine | | | 6.44 (d, 1H), 6.84 (d, |
| | | | | | 2H), 7.16 (d, 2H), |
| | | | | | 7.20 (dd, 1H), 7.30 (dd, 1H), |
| | | | | | 7.39 (dd, 1H), 7.68 (dd, |
| | | | | | 1H), 8.40 (bs, 1H) |
| 40.17 | 5-fluoro-2- | N-{[5-fluoro-2- | 54% | 506 [M + H]⁺ | 2.74 (d, 3H), 2.87 (dd, |
| | (trifluoromethyl) | (trifluoromethyl)phenyl] | | | 1H), 2.95 (t, 2H), |
| | | carbonyl}-O- | | | 3.10 (dd, 1H), 4.27 (t, 2H), |
| | | {2-[6- | | | 4.53 (ddd, 1H), 6.27 (d, |
| | | (methylamino)pyridin- | | | 1H), 6.36 (q, 1H), |
| | | 2-yl]ethyl}-L- | | | 6.43 (d, 1H), 6.87 (d, 2H), |
| | | tyrosine | | | 7.03 (dd, 1H), 7.18 (d, |
| | | | | | 2H), 7.30 (dd, 1H), |
| | | | | | 7.50 (dd, 1H), 7.85 (dd, 1H), |
| | | | | | 8.86 (bs, 1H) |
| 40.18 | 2- | O-{2-[6- | 62% | 488 [M + H]⁺ | 2.75 (d, 3H), 2.90 (dd, |
| | (trifluoromethyl) | (methylamino)pyridin- | | | 1H), 2.95 (t, 2H), |
| | | 2-yl]ethyl}-N- | | | 3.09 (dd, 1H), 4.27 (t, 2H), |
| | | {[2- | | | 4.45 (bs, 1H), 6.27 (d, |
| | | (trifluoromethyl)phenyl] | | | 1H), 6.36 (q, 1H), |
| | | carbonyl}-L- | | | 6.44 (d, 1H), 6.86 (d, 2H), |
| | | tyrosine | | | 7.17 (d, 2H), |
| | | | | | 7.28-7.34 (m, 2H), 7.63 (dd, 1H), |
| | | | | | 7.70 (dd, 1H), 7.75 (d, |
| | | | | | 1H), 8.58 (bs, 1H) |
| 40.19 | 2,5- | N-[(2,5- | 27% | 448 [M + H]⁺ | 2.12 (s, 3H), 2.25 (s, 3H), |
| | dimethyl | dimethylphenyl)carbonyl]- | | | 2.74 (d, 3H), 2.90 (dd, |
| | | O-{2-[6- | | | 1H), 2.94 (t, 2H), |
| | | (methylamino)pyridin- | | | 3.11 (dd, 1H), 4.26 (t, 2H), |
| | | 2-yl]ethyl}-L- | | | 4.36-4.44 (m, 1H), |
| | | tyrosine | | | 7.27 (d, 1H), 7.35 (q, 1H), |
| | | | | | 7.43 (d, 1H), 6.84 (d, |
| | | | | | 2H), 6.96 (s, 1H), |
| | | | | | 7.06 (d, 1H), 7.10 (d, 1H), |
| | | | | | 7.16 (d, 2H), 7.30 (dd, |
| | | | | | 1H), 8.07 (bs, 1H) |
| 40.20 | 2-ethyl | N-[(2- | 41% | 448 [M + H]⁺ | 1.00 (t, 3H), |
| | | ethylphenyl)carbonyl]- | | | 2.52-2.56 (m, partially hidden by |
| | | O-{2-[6- | | | DMSOd5, 2H), 2.75 (d, |
| | | (methylamino)pyridin- | | | 3H), 2.89 (dd, 1H), |
| | | 2-yl]ethyl}-L- | | | 2.94 (t, 2H), 3.11 (dd, 1H), |
| | | tyrosine | | | 4.26 (t, 2H), 4.46 (ddd, |
| | | | | | 1H), 7.27 (d, 1H), |
| | | | | | 7.36 (q, 1H), 7.43 (d, 1H), |
| | | | | | 6.85 (d, 2H), 7.13 (dd, |
| | | | | | 1H), 7.15-7.20 (m, 3H), |
| | | | | | 7.22 (d, 1H), |
| | | | | | 7.27-7.35 (m, 2H), 8.22 (d, 1H) |
| 40.21 | 3-chrolo-2- | N-[(3-chloro-2- | 36% | 468 [M + H]⁺ | 2.15 (s, 3H), 2.75 (d, 3H), |
| | methyl | methylphenyl)carbonyl]- | | | 2.86 (dd, 1H), 2.94 (t, |
| | | O-{2-[6- | | | 2H), 3.12 (dd, 1H), |
| | | (methylamino)pyridin- | | | 4.27 (t, 2H), 4.44 (ddd, 1H), |
| | | 2-yl]ethyl}-L- | | | 7.27 (d, 1H), 7.36 (q, 1H), |
| | | tyrosine | | | 7.44 (d, 1H), 6.85 (d, 2H), |
| | | | | | 7.08 (d, 1H), 7.17 (d, 2H), |
| | | | | | 7.24 (dd, 1H), 7.31 (dd, |
| | | | | | 1H), 7.47 (d, 1H), |
| | | | | | 8.37 (bs, 1H) |
| 40.22 | 4- | N-{[4- | 55% | 491 [M + H]⁺ | 2.04 (s, 3H), 2.16 (s, 3H), |
| | (acetylamino)- | (acetylamino)-2- | | | 2.75 (d, 3H), 2.89 (dd, |
| | 2- | methylphenyl]carbonyl}- | | | 1H), 2.94 (dd, 1H), |
| | methyl | O-{2-[6- | | | 3.09 (t, 2H), 4.26 (t, 2H), |
| | | (methylamino)pyridin- | | | 4.47 (ddd, 1H), 7.27 (d, 1H), |
| | | 2-yl]ethyl}-L- | | | 7.36 (q, 1H), 7.43 (d, |
| | | tyrosine | | | 1H), 6.86 (d, 2H), |
| | | | | | 7.15 (d, 1H), 7.18 (d, 2H), |
| | | | | | 7.31 (dd, 1H), 7.38 (s, |
| | | | | | 1H), 7.42 (d, 1H), |
| | | | | | 8.27 (d, 1H), 9.98 (s, 1H) |
| 40.23 | 2-fluoro-6- | N-{[2-fluoro-6- | 72% | 506 [M + H]⁺ | 2.75 (d, 3H), 2.87 (dd, |
| | (trifluoromethyl) | (trifluoromethyl)phenyl] | | | 1H), 2.95 (t, 2H), |
| | | carbonyl}-O- | | | 3.02 (dd, 1H), 4.27 (t, 2H), |
| | | {2-[6- | | | 4.56 (dd, 1H), 7.27 (d, |
| | | (methylamino)pyridin- | | | 1H), 7.36 (q, 1H), |
| | | 2-yl]ehtyl}-L- | | | 7.44 (d, 1H), 6.84 (d, 2H), |
| | | tyrosine | | | 7.16 (d, 2H), 7.31 (dd, |
| | | | | | 1H), 7.57-7.63 (m, 2H), |
| | | | | | 7.63-7.70 (m, 1H), |
| | | | | | 9.06 (bs, 1H) |
| 40.24 | 2-chloro-6- | N-[(2-chloro-6- | 32% | 486 [M + H]⁺ | 2.29 (s, 3H), 2.75 (d, |
| | fluoro-3- | fluoro-3- | | | 3H), 2.88 (dd, 1H), |
| | methyl | methylphenyl)carbonyl]- | | | 2.94 (t, 2H), 3.06 (dd, 1H), |
| | | O-{2-[6- | | | 4.26 (t, 2H), |
| | | (methylamino)pyridin- | | | 4.45-4.53 (m, 1H), 7.27 (d, 1H), |
| | | 2-yl]ethyl}-L- | | | 7.36 (q, 1H), 7.44 (d, |
| | | tyrosine | | | 1H), 6.83 (d, 2H), |
| | | | | | 7.13-7.20 (m, 3H), 7.31 (dd, |
| | | | | | 1H), 7.40 (dd, 1H), |
| | | | | | 8.76 (bs, 1H) |
| 40.25 | 4-chloro-2- | N-[(4-chloro-2- | 15% | 468 [M + H]⁺ | 2.18 (s, 3H), 2.74 (d, |
| | methyl | methylphenyl)carbonyl]- | | | 3H), 2.90 (dd, 1H), |
| | | O-{2-[6- | | | 2.94 (t, 2H), 3.12 (dd, 1H), |
| | | (methylamino)pyridin- | | | 4.25 (t, 2H), |
| | | 2-yl]ethyl}-L- | | | 4.28-4.35 (m, 1H), 7.27 (d, 1H), |
| | | tyrosine | | | 7.36 (q, 1H), 7.43 (d, |
| | | | | | 1H), 6.81 (d, 2H), |
| | | | | | 7.13 (d, 2H), 7.17 (d, 1H), |
| | | | | | 7.26 (dd, 1H), 7.29 (s, |
| | | | | | 1H), 7.31 (d, 1H), |
| | | | | | 7.97 (bs, 1H) |
| 40.26 | 2- | N-{[2- | 18% | 511 [M + H]⁺ | 1.85 (s, 3H), 2.75 (d, |
| | (acetylamino)- | (acetylamino)-6- | | | 3H), 2.91-2.99 (m, 3H), |
| | 6- | chlorophenyl]carbonyl}- | | | 3.09 (dd, 1H), 4.26 (t, |
| | chloro | O-{2-[6- | | | 2H), 4.40 (ddd, 1H), |
| | | (methylamino)pyridin- | | | 6.28 (d, 1H), 6.40 (bs, 1H), |
| | | 2-yl]ethyl}-L- | | | 6.45 (d, 1H), 6.83 (d, |
| | | tyrosine | | | 2H), 7.16-7.23 (m, 3H), |
| | | | | | 7.32 (dd, 1H), 8.06 (d, |
| | | | | | 1H), 8.39 (d, 1H), |
| | | | | | 11.24 (bs, 1H) |
| 40.27 | 2,3- | N-[(2,3- | 61% | 448 [M + H]⁺ | 2.06 (s, 3H), 2.22 (s, 3H), |
| | dimethyl | dimethylphenyl)carbonyl]- | | | 2.75 (d, 3H), 2.91 (dd, |
| | | O-{2-[6- | | | 1H), 2.94 (t, 2H), |
| | | (methylamino)pyridin- | | | 3.11 (dd, 1H), 4.26 (t, 2H), |
| | | 2-yl]ethyl}-L- | | | 4.31-4.38 (m, 1H), |
| | | tyrosine | | | 6.27 (d, 1H), 6.36 (q, 1H), |
| | | | | | 6.44 (d, 1H), 6.83 (d, |
| | | | | | 2H), 6.94 (d, 1H), |
| | | | | | 7.07 (dd, 1H), 7.14 (d, 2H), |
| | | | | | 7.17 (d, 1H), 7.31 (dd, |
| | | | | | 1H), 7.91 (bs, 1H) |
| 40.28 | 2,4- | N-[(2,4- | 65% | 448 [M + H]⁺ | 2.16 (s, 3H), 2.26 (s, 3H), |
| | dimethyl | dimethylphenyl)carbonyl]- | | | 2.74 (d, 3H), |
| | | O-{2-[6- | | | 2.88-2.96 (m, 3H), 3.11 (dd, 1H), |
| | | (methylamino)pyridin- | | | 4.25 (t, 2H), |
| | | 2-yl]ethyl}-L- | | | 4.32-4.38 (m, 1H), 6.27 (d, 1H), |
| | | tyrosine | | | 6.36 (q, 1H), 6.43 (d, |
| | | | | | 1H), 6.82 (d, 2H), |
| | | | | | 6.99 (d, 1H), 7.00 (s, 1H), |
| | | | | | 7.08 (d, 1H), 7.14 (d, |
| | | | | | 2H), 7.30 (dd, 1H), |
| | | | | | 7.92 (bs, 1H) |
| 40.29 | 4-methoxy- | N-[(4-methoxy-2- | 65% | 464 [M + H]⁺ | 2.22 (s, 3H), 2.74 (d, |
| | 2-methyl | methylphenyl)carbonyl]- | | | 3H), 2.89-2.97 (m, 3H), |
| | | O-{2-[6- | | | 3.11 (dd, 1H), 3.74 (s, |
| | | (methylamino)pyridin- | | | 3H), 4.25 (t, 2H), |
| | | 2-yl]ethyl}-L- | | | 4.29 (ddd, 1H), 6.27 (d, 1H), |
| | | tyrosine | | | 6.36 (q, 1H), 6.43 (d, |
| | | | | | 1H), 7.72-7.77 (m, 2H), |
| | | | | | 6.80 (d, 2H), 7.12 (d, |
| | | | | | 2H), 7.16 (d, 1H), |
| | | | | | 7.30 (dd, 1H), 7.71 (d, 1H) |
| 40.30 | 4-fluoro-2- | N-{[4-fluoro-2- | 69% | 506 [M + H]⁺ | 2.74 (d, 3H), 2.87 (dd, |
| | (trifluoromethyl) | (trifluoromethyl)phenyl] | | | 1H), 2.95 (t, 2H), |
| | | carbonyl}-O- | | | 3.09 (dd, 1H), 4.27 (t, 2H), |
| | | {2-[6- | | | 4.52 (ddd, 1H), 6.27 (d, |
| | | (methylamino)pyridin- | | | 1H), 6.36 (q, 1H), |
| | | 2-yl]ethyl}-L- | | | 6.44 (d, 1H), 6.87 (d, 2H), |
| | | tyrosine | | | 7.18 (d, 2H), 7.31 (dd, |
| | | | | | 1H), 7.38 (dd, 1H), |
| | | | | | 7.61 (ddd, 1H), 7.69 (dd, 1H), |
| | | | | | 8.82 (d, 1H) |
| 40.31 | 5-fluoro-2- | N-[(5-fluoro-2- | 69% | 452 [M + H]⁺ | 2.12 (s, 3H), 2.74 (d, |
| | methyl | methylphenyl)carbonyl]- | | | 3H), 2.87 (dd, 1H), |
| | | O-{2-[6- | | | 2.94 (t, 2H), 3.11 (dd, 1H), |
| | | (methylamino)pyridin- | | | 4.27 (t, 2H), 4.52 (ddd, |
| | | 2-yl]ethyl}-L- | | | 1H), 6.27 (d, 1H), |
| | | tyrosine | | | 6.35 (q, 1H), 6.43 (d, 1H), |
| | | | | | 6.86 (d, 2H), 6.90 (dd, |
| | | | | | 1H), 7.16 (ddd, 1H), |
| | | | | | 7.19 (d, 2H), 7.24 (dd, 1H), |
| | | | | | 7.30 (dd, 1H), 8.54 (d, |
| | | | | | 1H) |
| 40.32 | 3,6- | N-[(3,6-dichloro-2- | 55% | 518 [M + H]⁺ | 2.75 (d, 3H), 2.87 (dd, |
| | dichloro-2- | methoxyphenyl)carbonyl]- | | | 1H), 2.95 (t, 2H), |
| | methoxy | O-{2-[6- | | | 3.07 (dd, 1H), 3.65 (s, 3H), |
| | | (methylamino)pyridin- | | | 4.27 (t, 2H), 4.60 (ddd, |
| | | 2-yl]ethyl}-L- | | | 1H), 6.27 (d, 1H), |
| | | tyrosine | | | 6.36 (q, 1H), 6.44 (d, 1H), |
| | | | | | 6.85 (d, 2H), 7.19 (d, |
| | | | | | 2H), 7.25 (d, 1H), |
| | | | | | 7.31 (dd, 1H), 7.52 (d, 1H), |
| | | | | | 8.90 (d, 1H) |
| 40.33 | 2-chloro-5- | N-{[2-chloro-5- | 70% | 522 [M + H]⁺ | 2.75 (d, 3H), 2.87 (dd, |
| | (trifluoromethyl) | (trifluoromethyl)phenyl] | | | 1H), 2.95 (t, 2H), |
| | | carbonyl}-O- | | | 3.13 (dd, 1H), 4.26 (t, 2H), |
| | | {2-[6- | | | 4.58 (ddd, 1H), 6.27 (d, |
| | | (methylamino)pyridin- | | | 1H), 6.35 (q, 1H), |
| | | 2-yl]ethyl}-L- | | | 6.43 (d, 1H), 6.86 (d, 2H), |
| | | tyrosine | | | 7.20 (d, 2H), 7.30 (dd, |
| | | | | | 1H), 7.44 (d, 1H), |
| | | | | | 7.73 (d, 1H), 7.82 (dd, 1H), |
| | | | | | 8.95 (d, 1H) |
| 40.34 | 3-fluoro-2- | N-[(3-fluoro-2- | 71% | 452 [M + H]⁺ | 2.05 (s, 3H), 2.74 (d, |
| | methyl | methylphenyl)carbonyl]- | | | 3H), 2.88 (dd, 1H), |
| | | O-{2-[6- | | | 2.94 (t, 2H), 3.11 (dd, 1H), |
| | | (methylamino)pyridin- | | | 4.26 (t, 2H), |
| | | 2-yl]ethyl}-L- | | | 4.44-4.52 (m, 1H), 6.27 (d, 1H), |
| | | tyrosine | | | 6.35 (q, 1H), 6.43 (d, |
| | | | | | 1H), 6.86 (d, 2H), |
| | | | | | 6.99 (d, 1H), 7.18 (d, 2H), |
| | | | | | 7.21 (d, 1H), |
| | | | | | 7.22-7.28 (m, 1H), 7.30 (dd, 1H), |
| | | | | | 8.43 (bs, 1H) |
| 40.35 | 2,4- | N-[(2,4-dichloro-5- | 67% | 506 [M + H]⁺ | 2.74 (d, 3H), 2.89 (dd, |
| | dichloro-5- | fluorophenyl)carbonyl]- | | | 1H), 2.94 (t, 2H), |
| | fluoro | O-{2-[6- | | | 3.11 (dd, 1H), 4.26 (t, 2H), |
| | | (methylamino)pyridin- | | | 4.39-4.46 (m, 1H), |
| | | 2-yl]ethyl}-L- | | | 6.26 (d, 1H), 6.35 (q, 1H), |
| | | tyrosine | | | 6.43 (d, 1H), 6.84 (d, |
| | | | | | 2H), 7.16 (d, 2H), |
| | | | | | 7.25 (d, 1H), 7.30 (dd, 1H), |
| | | | | | 7.85 (d, 1H), 8.56 (bs, |
| | | | | | 1H) |
| 40.36 | 2,4,5- | O-{2-[6- | 71% | 462 [M + H]⁺ | 2.11 (s, 3H), 2.16 (s, 3H), |
| | trimethyl | (methylamino)pyridin- | | | 2.18 (s, 3H), 2.75 (d, |
| | | 2-yl]ethyl}-N- | | | 3H), 2.90 (dd, 1H), |
| | | [(2,4,5- | | | 2.94 (t, 2H), 3.10 (dd, 1H), |
| | | trimethylphenyl)carbonyl]- | | | 4.26 (t, 2H), |
| | | L-tyrosine | | | 4.39-4.46 (m, 1H), 6.27 (d, 1H), |
| | | | | | 6.35 (q, 1H), 6.43 (d, |
| | | | | | 1H), 6.84 (d, 2H), |
| | | | | | 6.95 (s, 2H), 7.16 (d, 2H), |
| | | | | | 7.30 (dd, 1H), 8.05 (bs, |
| | | | | | 1H) |
| 40.37 | 2-chloro- | N-[(2-chloro-4,5- | 67% | 490 [M + H]⁺ | 2.74 (d, 3H), 2.90 (dd, |
| | 4,5- | difluorophenyl)carbonyl]- | | | 1H), 2.94 (t, 2H), |
| | difluoro | O-{2-[6- | | | 3.12 (dd, 1H), 4.25 (t, 2H), |
| | | (methylamino)pyridin- | | | 4.28 (dd, 1H), 6.27 (d, |
| | | 2-yl]ethyl}-L- | | | 1H), 6.35 (q, 1H), |
| | | tyrosine | | | 6.43 (d, 1H), 6.81 (d, 2H), |
| | | | | | 7.13 (d, 2H), |
| | | | | | 7.27-7.36 (m, 2H), 7.74 (dd, 1H), |
| | | | | | 8.18 (bs, 1H) |
| 40.38 | 2-chloro- | N-[(2-chloro-3,4- | 68% | 514 [M + H]⁺ | 2.74 (d, 3H), 2.92 (dd, |
| | 3,4- | dimethoxyphenyl)carbonyl]- | | | 1H), 2.94 (t, 2H), |
| | dimethoxy | O-{2-[6- | | | 3.09 (dd, 1H), 3.73 (s, 3H), |
| | | (methylamino)pyridin- | | | 3.84 (s, 3H), 4.26 (t, 2H), |
| | | 2-yl]ethyl}-L- | | | 4.31-4.39 (m, 1H), |
| | | tyrosine | | | 6.27 (d, 1H), 6.36 (q, 1H), |
| | | | | | 6.43 (d, 1H), 6.83 (d, |
| | | | | | 2H), 7.03 (d, 1H), |
| | | | | | 7.07 (d, 1H), 7.15 (d, 2H), |
| | | | | | 7.30 (dd, 1H), 8.18 (bs, |
| | | | | | 1H) |
| 40.39 | 2-chloro-4- | N-{[2-chloro-4- | 63% | 532 [M + H]⁺ | 2.75 (d, 3H), 2.90 (dd, |
| | (methylsulfonyl) | (methylsulfonyl)phenyl] | | | 1H), 2.94 (t, 2H), |
| | | carbonyl}-O- | | | 3.13 (dd, 1H), 3.29 (s, 3H), |
| | | {2-[6- | | | 4.26 (t, 2H), |
| | | (methylamino)pyridin- | | | 4.36-4.43 (m, 1H), 6.27 (d, 1H), |
| | | 2-yl]ethyl}-L- | | | 6.35 (q, 1H), 6.44 (d, |
| | | tyrosine | | | 1H), 6.84 (d, 2H), |
| | | | | | 7.16 (d, 2H), 7.31 (dd, 1H), |
| | | | | | 7.50 (d, 1H), 7.92 (dd, |
| | | | | | 1H), 7.99 (d, 1H) |
| | | | | | 8.50 (bs, 1H) |
| 40.40 | 3-hydroxy- | N-[(3-hydroxy-2- | 82% | 522 [M + H]⁺ | 1.98 (s, 3H), 2.75 (d, |
| | 2-methyl | methylphenyl)carbonyl]- | | | 3H), 2.91 (dd, 1H), |
| | | O-{2-[6- | | | 2.94 (t, 2H), 3.11 (dd, 1H), |
| | | (methylamino)pyridin- | | | 4.26 (t, 2H), 4.31 (ddd, |
| | | 2-yl]ethyl}-L- | | | 1H), 6.27 (d, 1H), |
| | | tyrosine | | | 6.35 (q, 1H), 6.43 (d, 1H), |
| | | | | | 6.59 (dd, 1H), |
| | | | | | 6.79-6.84 (m, 3H), 6.97 (dd, 1H), |
| | | | | | 7.13 (d, 2H), 7.30 (dd, |
| | | | | | 1H), 7.77 (bs, 1H), |
| | | | | | 9.50 (bs, 1H) |
| 40.41 | 2-chloro-3- | N-{[2-chloro-3- | 69% | 522 [M + H]⁺ | 2.74 (d, 3H), 2.88 (dd, |
| | (trifluoromethyl) | (trifluoromethyl)phenyl] | | | 1H), 2.95 (t, 2H), |
| | | carbonyl}-O- | | | 3.12 (dd, 1H), 4.27 (t, 2H), |
| | | {2-[6- | | | 4.48 (ddd, 1H), 6.27 (d, |
| | | (methylamino)pyridin- | | | 1H), 6.36 (q, 1H), |
| | | 2-yl]ethyl}-L- | | | 6.43 (d, 1H), 6.86 (d, 2H), |
| | | tyrosine | | | 7.18 (d, 2H), 7.30 (dd, |
| | | | | | 1H), 7.49 (dd, 1H), |
| | | | | | 7.57 (dd, 1H), 7.90 (dd, 1H), |
| | | | | | 8.68 (bs, 1H) |
| 40.42 | 3-chloro-2- | N-{[3-chloro-2- | 69% | 540 [M + H]⁺ | 2.75 (d, 3H), 2.87 (dd, |
| | fluoro-6- | fluoro-6- | | | 1H), 2.95 (t, 2H), |
| | (trifluoromethyl) | (trifluoromethyl)phenyl] | | | 3.03 (dd, 1H), 4.27 (t, 2H), |
| | | carbonyl}-O- | | | 4.50-4.58 (m, 1H), |
| | | {2-[6- | | | 6.27 (d, 1H), 6.36 (q, 1H), |
| | | (methylamino)pyridin- | | | 6.43 (d, 1H), 6.84 (d, |
| | | 2-yl]ethyl}-L- | | | 2H), 7.15 (d, 2H), |
| | | tyrosine | | | 7.31 (dd, 1H), 7.63 (d, 1H), |
| | | | | | 7.87 (dd, 1H), 9.05 (s, |
| | | | | | 1H) |
| 40.43 | 5-chloro-2- | N-{[5-chloro-2- | 70% | 522 [M + H]⁺ | 2.74 (d, 3H), 2.87 (dd, |
| | (trifluoromethyl) | (trifluoromethyl)phenyl] | | | 1H), 2.95 (t, 2H), |
| | | carbonyl}-O- | | | 3.11 (dd, 1H), 4.28 (t, 2H), |
| | | {2-[6- | | | 4.53 (ddd, 1H), 6.27 (d, |
| | | (methylamino)pyridin- | | | 1H), 6.35 (q, 1H), |
| | | 2-yl]ethyl}-L- | | | 6.43 (d, 1H), 6.87 (d, 2H), |
| | | tyrosine | | | 7.18 (d, 2H), 7.22 (d, |
| | | | | | 1H), 7.30 (dd, 1H), |
| | | | | | 7.72 (dd, 1H), 7.80 (d, 1H), |
| | | | | | 8.87 (d, 1H) |
| 40.44 | 3-methoxy- | N-[(3-methoxy-2- | 81% | 464 [M + H]⁺ | 1.99 (s, 3H), 2.75 (d, |
| | 2-methyl | methylphenyl)carbonyl]- | | | 3H), 2.90 (dd, 1H), |
| | | O-{2-[6- | | | 2.94 (t, 2H), 3.11 (dd, 1H), |
| | | (methylamino)pyridin- | | | 3.78 (s, 3H), 4.26 (t, 2H), |
| | | 2-yl]ethyl}-L- | | | 4.34 (ddd, 1H), 6.27 (d, |
| | | tyrosine | | | 1H), 6.36 (q, 1H), |
| | | | | | 6.44 (d, 1H), 6.72 (d, 1H), |
| | | | | | 6.82 (d, 2H), 6.97 (d, |
| | | | | | 1H), 7.14 (d, 2H), |
| | | | | | 7.16 (dd, 1H), 7.31 (dd, 1H), |
| | | | | | 7.91 (bs, 1H) |
| 40.45 | 4-fluoro-2- | N-[(4-fluoro-2- | 70% | 452 [M + H]⁺ | 2.21 (s, 3H), 2.74 (d, |
| | methyl | methylphenyl)carbonyl]- | | | 3H), 2.91 (dd, 1H), |
| | | O-{2-[6- | | | 2.94 (t, 2H), 3.12 (dd, 1H), |
| | | (methylamino)pyridin- | | | 4.25 (t, 2H), 4.28 (dd, |
| | | 2-yl]ethyl}-L- | | | 1H), 6.27 (d, 1H), |
| | | tyrosine | | | 6.35 (q, 1H), 6.43 (d, 1H), |
| | | | | | 6.81 (d, 2H), 7.02 (ddd, |
| | | | | | 1H), 7.05 (dd, 1H), |
| | | | | | 7.12 (d, 2H), 7.21 (dd, 1H), |
| | | | | | 7.30 (dd, 1H), 7.85 (bs, |
| | | | | | 1H) |
| 40.46 | 2-chloro-4- | N-[(2-chloro-4- | 37% | 551 [M + H]⁺ | 2.75 (d, 3H), 2.90 (dd, |
| | fluoro-5- | fluoro-5- | | | 1H), 2.94 (t, 2H), |
| | sulfamoyl | sulfamoylphenyl)carbonyl]- | | | 3.12 (dd, 1H), 4.26 (t, 2H), |
| | | O-{2-[6- | | | 4.31-4.38 (m, 1H), |
| | | (methylamino)pyridin- | | | 6.27 (d, 1H), 6.35 (q, 1H), |
| | | 2-yl]ethyl}-L- | | | 6.44 (d, 1H), 6.82 (d, |
| | | tyrosine | | | 2H), 7.14 (d, 2H), |
| | | | | | 7.31 (dd, 1H), 7.56 (bs, 2H), |
| | | | | | 7.73 (d, 1H), 7.75 (d, |
| | | | | | 1H), 8.47 (s, 1H) |
| 40.47 | 2-chloro-3- | N-[(2-chloro-3- | 66% | 468 [M + H]⁺ | 2.33 (s, 3H), 2.75 (d, |
| | methyl | methylphenyl)carbonyl]- | | | 3H), 2.89-2.97 (m, 3H), |
| | | O-{2-[6- | | | 3.11 (dd, 1H), 4.26 (t, |
| | | (methylamino)pyridin- | | | 2H), 4.32-4.38 (m, 1H), |
| | | 2-yl]ethyl}-L- | | | 6.27 (d, 1H), 6.36 (q, |
| | | tyrosine | | | 1H), 6.44 (d, 1H), |
| | | | | | 6.82 (d, 2H), 7.08 (dd, 1H), |
| | | | | | 7.15 (d, 2H), 7.25 (dd, |
| | | | | | 1H), 7.30 (dd, 1H), |
| | | | | | 7.38 (dd, 1H), 8.14 (bs, 1H) |
| 40.48 | 2-chloro-4- | N-[(2-chloro-4- | 64% | 484 [M + H]⁺ | 2.74 (d, 3H), |
| | methoxy | methoxyphenyl)carbonyl]- | | | 2.89-2.96 (m, 3H), 3.10 (dd, 1H), |
| | | O-{2-[6- | | | 3.79 (s, 3H), 4.25 (t, 2H), |
| | | (methylamino)pyridin- | | | 4.35-4.41 (m, 1H), |
| | | 2-yl]ethyl}-L- | | | 6.27 (d, 1H), 6.35 (q, 1H), |
| | | tyrosine | | | 6.43 (d, 1H), 6.83 (d, |
| | | | | | 2H), 6.94 (dd, 1H), |
| | | | | | 7.02 (d, 1H), 7.16 (d, 2H), |
| | | | | | 7.27 (d, 1H), 7.30 (dd, |
| | | | | | 1H), 8.23 (bs, 1H) |
| 40.49 | 2-chloro-4- | N-[(2-chloro-4- | 38% | 523 [M + H]⁺ | 1.91-1.99 (m, 4H), |
| | pyrrolidin- | pyrrolidin-1- | | | 2.74 (d, 3H), 2.91-2.98 (m, |
| | 1-yl | ylphenyl)carbonyl]- | | | 3H), 3.08 (dd, 1H), |
| | | O-{2-[6- | | | 3.21-3.27 (m, 4H), 4.25 (t, |
| | | (methylamino)pyridin- | | | 2H), 4.35-4.41 (m, 1H), |
| | | 2-yl]ethyl}-L- | | | 6.27 (d, 1H), 6.35 (q, |
| | | tyrosine | | | 1H), 6.43 (d, 1H), |
| | | | | | 6.45-6.50 (m, 2H), 6.81 (d, |
| | | | | | 2H), 7.14 (d, 2H), |
| | | | | | 7.28 (d, 1H), 7.30 (dd, 1H), |
| | | | | | 7.95 (d, 1H |
| 40.50 | 2-chloro-4- | N-[(2-chloro-4- | 62% | 539 [M + H]⁺ | 2.74 (d, 3H), |
| | morpholin- | morpholin-4- | | | 2.89-2.93 (m, 3H), 3.08 (dd, 1H), |
| | 4-yl | ylphenyl)carbonyl]- | | | 3.16-3.20 (m, 4H), |
| | | O-{2-[6- | | | 3.69-3.74 (m, 4H), 4.25 (t, |
| | | (methylamino)pyridin- | | | 2H), 4.36-4.43 (m, 1H), |
| | | 2-yl]ethyl}-L- | | | 6.27 (d, 1H), 6.35 (q, |
| | | tyrosine | | | 1H), 6.43 (d, 1H), |
| | | | | | 6.83 (d, 2H), 6.88-6.94 (m, |
| | | | | | 2H), 7.15 (d, 2H), |
| | | | | | 7.24 (d, 1H), 7.30 (dd, 1H), |
| | | | | | 8.15 (d, 1H |
| 40.51 | 2-chloro-4- | N-{[2-chloro-4- | 59% | 520 [M + H]⁺ | 2.74 (d, 3H), 2.92 (dd, |
| | (1H- | (1H-pyrazol-1- | | | 1H), 2.95 (t, 2H), |
| | pyrazol-1- | yl)phenyl]carbonyl}- | | | 3.12 (dd, 1H), 4.27 (t, 2H), |
| | yl) | O-{2-[6- | | | 4.43-4.50 (m, 1H), |
| | | (methylamino)pyridin- | | | 6.27 (d, 1H), 6.35 (q, 1H), |
| | | 2-yl]ethyl}-L- | | | 6.44 (d, 1H), 6.60 (dd, |
| | | tyrosine | | | 1H), 6.86 (d, 2H), |
| | | | | | 7.19 (d, 2H), 7.31 (dd, 1H), |
| | | | | | 7.40 (d, 1H), 7.81 (d, |
| | | | | | 1H), 7.88 (dd, 1H), |
| | | | | | 7.97 (d, 1H), 8.57 (bs, 1H), |
| | | | | | 8.62 (d, 1H) |
| 40.52 | 2-chloro-5- | N-[(2-chloro-5- | 66% | 484 [M + H]⁺ | 2.74 (d, 3H), 2.89 (dd, |
| | methoxy | methoxyphenyl)carbonyl]- | | | 1H), 2.94 (t, 2H), |
| | | O-{2-[6- | | | 3.11 (dd, 1H), 3.74 (s, 3H), |
| | | (methylamino)pyridin- | | | 4.26 (t, 2H), 4.48 (ddd, |
| | | 2-yl]ethyl}-L- | | | 1H), 6.27 (d, 1H), |
| | | tyrosine | | | 6.35 (q, 1H), 6.43 (d, 1H), |
| | | | | | 6.70 (d, 1H), 6.85 (d, |
| | | | | | 2H), 7.00 (dd, 1H), |
| | | | | | 7.19 (d, 2H), 7.30 (dd, 1H), |
| | | | | | 7.35 (d, 1H), 8.53 (d, 1H) |
| 40.53 | 2-chloro- | N-[(2-chloro-4,5- | 57% | 514 [M + H]⁺ | 2.74 (d, 3H), |
| | 4,5- | dimethoxyphenyl)carbonyl]- | | | 2.89-2.97 (m, 3H), 3.11 (dd, 1H), |
| | dimethoxy | O-{2-[6- | | | 3.72 (s, 3H), 3.79 (s, 3H), |
| | | (methylamino)pyridin- | | | 4.25 (t, 2H), |
| | | 2-yl]ethyl}-L- | | | 4.39-4.47 (m, 1H), 6.27 (d, 1H), |
| | | tyrosine | | | 6.35 (q, 1H), 6.43 (d, |
| | | | | | 1H), 6.76 (s, 1H), |
| | | | | | 6.84 (d, 2H), 7.00 (s, 1H), |
| | | | | | 7.18 (d, 2H), 7.30 (dd, |
| | | | | | 1H), 8.27 (bs, 1H) |
| 40.54 | 2-methyl- | O-{2-[6- | 72% | 502 [M + H]⁺ | 2.23 (s, 3H), 2.75 (d, |
| | 3- | (methylamino)pyridin- | | | 3H), 2.86 (dd, 1H), |
| | (trifluoromethyl) | 2-yl]ethyl}-N- | | | 2.94 (t, 2H), 3.14 (dd, 1H), |
| | | {[2-methyl-3- | | | 4.27 (t, 2H), 4.44 (ddd, |
| | | (trifluoromethyl)phenyl] | | | 1H), 6.27 (d, 1H), |
| | | carbonyl}-L- | | | 6.35 (q, 1H), 6.43 (d, 1H), |
| | | tyrosine | | | 6.85 (d, 2H), 7.17 (d, |
| | | | | | 2H), 7.30 (dd, 1H), |
| | | | | | 7.34 (d, 1H), 7.42 (dd, 1H), |
| | | | | | 7.71 (d, 1H), 8.40 (bs, |
| | | | | | 1H) |
| 40.55 | 5-chloro-2- | N-[(5-chloro-2- | 66% | 468 [M + H]⁺ | 2.12 (s, 3H), 2.74 (d, |
| | methyl | methylphenyl)carbonyl]- | | | 3H), 2.87 (dd, 1H), |
| | | O-{2-[6- | | | 2.94 (t, 2H), 3.12 (dd, 1H), |
| | | (methylamino)pyridin- | | | 4.27 (t, 2H), 4.50 (ddd, |
| | | 2-yl]ethyl}-L- | | | 1H), 6.27 (d, 1H), |
| | | tyrosine | | | 6.35 (q, 1H), 6.43 (d, 1H), |
| | | | | | 6.85 (d, 2H), 7.11 (d, |
| | | | | | 1H), 7.18 (d, 2H), |
| | | | | | 7.23 (d, 1H), 7.30 (dd, 1H), |
| | | | | | 7.37 (dd, 1H), 8.50 (d, |
| | | | | | 1H) |
| 40.56 | 2-methyl- | O-{2-[6- | 65% | 502 [M + H]⁺ | 2.22 (s, 3H), 2.74 (d, |
| | 5- | (methylamino)pyridin- | | | 3H), 2.88 (dd, 1H), |
| | (trifluoromethyl) | 2-yl]ethyl}-N- | | | 2.94 (t, 2H), 3.14 (dd, 1H), |
| | | {[2-methyl-5- | | | 4.26 (t, 2H), 4.48 (bs, |
| | | (trifluoromethyl)phenyl] | | | 1H), 6.27 (d, 1H), |
| | | carbonyl}-L- | | | 6.35 (q, 1H), 6.43 (d, 1H), |
| | | tyrosine | | | 6.84 (d, 2H), 7.17 (d, |
| | | | | | 2H), 7.30 (dd, 1H), |
| | | | | | 7.38 (s, 1H), 7.44 (d, 1H), |
| | | | | | 7.66 (dd, 1H), 8.50 (bs, |
| | | | | | 1H) |
| 40.57 | 3-fluoro-2- | N-{[3-fluoro-2- | 70% | 506 [M + H]⁺ | 2.75 (d, 3H), 2.86 (dd, |
| | (trifluoromethyl) | (trifluoromethyl)phenyl] | | | 1H), 2.95 (t, 2H), |
| | | carbonyl}-O- | | | 3.09 (dd, 1H), 4.27 (t, 2H), |
| | | {2-[6- | | | 4.44 (ddd, 1H), 6.27 (d, |
| | | (methylamino)pyridin- | | | 1H), 6.36 (q, 1H), |
| | | 2-yl]ethyl}-L- | | | 6.44 (d, 1H), 6.86 (d, 2H), |
| | | tyrosine | | | 7.07 (d, 1H), 7.16 (d, |
| | | | | | 2H), 7.31 (dd, 1H), |
| | | | | | 7.53 (dd, 1H), 7.75 (ddd, 1H), |
| | | | | | 8.68 (bs, 1H) |
| 40.58 | 4-methoxy- | N-{[4-methoxy-2- | 69% | 518 [M + H]⁺ | 2.74 (d, 3H), 2.88 (dd, |
| | 2- | (trifluoromethyl)phenyl] | | | 1H), 2.95 (t, 2H), |
| | (trifluoromethyl) | carbonyl}-O- | | | 3.08 (dd, 1H), 3.85 (s, 3H), |
| | | {2-[6- | | | 4.27 (t, 2H), |
| | | (methylamino)pyridin- | | | 4.44-4.51 (m, 1H), 6.27 (d, 1H), |
| | | 2-yl]ethyl}-L- | | | 6.35 (q, 1H), 6.43 (d, |
| | | tyrosine | | | 1H), 6.86 (d, 2H), |
| | | | | | 7.18 (d, 2H), 7.22 (d, 1H), |
| | | | | | 7.23-7.29 (m, 2H), |
| | | | | | 7.31 (d, 1H), 8.62 (bs, 1H) |
| 40.59 | 2-chloro-3- | N-[(2-chloro-3- | 30% | 472 [M + H]⁺ | 2.75 (d, 3H), 2.88 (dd, |
| | fluoro | fluorophenyl)carbonyl]- | | | 1H), 2.95 (t, 2H), |
| | | O-{2-[6- | | | 3.10 (dd, 1H), 4.27 (t, 2H), |
| | | (methylamino)pyridin- | | | 4.51 (ddd, 1H), 6.27 (d, |
| | | 2-yl]ethyl}-L- | | | 1H), 6.35 (q, 1H), |
| | | tyrosine | | | 6.43 (d, 1H), 6.86 (d, 2H), |
| | | | | | 7.10 (d, 1H), 7.19 (d, |
| | | | | | 2H), 7.31 (dd, 1H), |
| | | | | | 7.43 (ddd, 1H), 7.47 (ddd, |
| | | | | | 1H), 8.75 (d, 1H) |
| 40.60 | 2-chloro-5- | N-[(2-chloro-5- | 42% | 472 [M + H]⁺ | 2.75 (d, 3H), 2.88 (dd, |
| | fluoro | fluorophenyl)carbonyl]- | | | 1H), 2.95 (t, 2H), |
| | | O-{2-[6- | | | 3.10 (dd, 1H), 4.27 (t, 2H), |
| | | (methylamino)pyridin- | | | 4.51 (ddd, 1H), 6.27 (d, |
| | | 2-yl]ethyl}-L- | | | 1H), 6.35 (q, 1H), |
| | | tyrosine | | | 6.43 (d, 1H), 6.86 (d, 2H), |
| | | | | | 7.02 (dd, 1H), 7.19 (d, |
| | | | | | 2H), 7.30 (dd, 1H), |
| | | | | | 7.33 (ddd, 1H), 7.52 (dd, 1H), |
| | | | | | 8.75 (d, 1H) |
| 40.61 | 2-ethyl-4- | N-[(2-ethyl-4- | 42% | 466 [M + H]⁺ | 1.01 (t, 3H), 2.55 (q, 2H), |
| | fluoro | fluorophenyl)carbonyl]- | | | 2.74 (d, 3H), 2.89 (dd, |
| | | O-{2-[6- | | | 1H), 2.93 (t, 2H), |
| | | (methylamino)pyridin- | | | 3.13 (dd, 1H), 4.24 (t, 2H), |
| | | 2-yl]ethyl}-L- | | | 4.29 (ddd, 1H), 6.26 (d, |
| | | tyrosine | | | 1H), 6.36 (q, 1H), |
| | | | | | 6.42 (d, 1H), 6.80 (d, 2H), |
| | | | | | 7.00 (ddd, 1H), 7.05 (dd, |
| | | | | | 1H), 7.12 (d, 2H), |
| | | | | | 7.17 (dd, 1H), 7.29 (dd, 1H), |
| | | | | | 7.77 (d, 1H) |
| 40.62 | 4,5- | N-[(4,5-difluoro-2- | 43% | 470 [M + H]⁺ | 2.15 (s, 3H), 2.74 (d, |
| | difluoro-2- | methylphenyl)carbonyl]- | | | 3H), 2.87 (dd, 1H), |
| | methyl | O-{2-[6- | | | 2.93 (t, 2H), 3.13 (dd, 1H), |
| | | (methylamino)pyridin- | | | 4.24 (t, 2H), 4.27 (ddd, |
| | | 2-yl]ethyl}-L- | | | 1H), 6.26 (d, 1H), |
| | | tyrosine | | | 6.36 (q, 1H), 6.42 (d, 1H), |
| | | | | | 6.80 (d, 2H), 7.11 (d, |
| | | | | | 2H), 7.13 (d, 1H), |
| | | | | | 7.24-7.33 (m, 2H), 7.87 (d, |
| | | | | | 1H) |
| 40.63 | 2-ethyl-5- | N-[(2-ethyl-5- | 43% | 466 [M + H]⁺ | 0.98 (t, 3H), 2.52 (q |
| | fluoro | fluorophenyl)carbonyl]- | | | partially hidden by |
| | | O-{2-[6- | | | DMSOd6, 2H), 2.74 (d, |
| | | (methylamino)pyridin- | | | 3H), 2.87 (dd, 1H), |
| | | 2-yl]ethyl}-L- | | | 2.93 (t, 2H), 3.14 (dd, 1H), |
| | | tyrosine | | | 4.25 (t, 2H), 4.36 (ddd, |
| | | | | | 1H), 6.26 (d, 1H), |
| | | | | | 6.35 (q, 1H), 6.42 (d, 1H), |
| | | | | | 6.82 (d, 2H), 6.86 (dd, |
| | | | | | 1H), 7.12-7.18 (m, 3H), |
| | | | | | 7.24 (dd, 1H), 7.29 (dd, |
| | | | | | 1H), 8.06 (d, 1H) |
| 40.64 | 2- | N-[(2-cyclopropyl- | 57% | 478 [M + H]⁺ | 0.56-0.65 (m, 2H), |
| | cyclopropyl- | 4- | | | 0.70-0.81 (m, 2H), |
| | 4-fluoro | fluorophenyl)carbonyl]- | | | 1.89-1.98 (m, 1H), 2.74 (d, 3H), |
| | | O-{2-[6- | | | 2.89 (dd, 1H), 2.94 (t, |
| | | (methylamino)pyridin- | | | 2H), 3.12 (dd, 1H), |
| | | 2-yl]ethyl}-L- | | | 4.25 (t, 2H), 4.52 (dd, 1H), |
| | | tyrosine | | | 6.26 (d, 1H), 6.36 (q, |
| | | | | | 1H), 6.43 (d, 1H), |
| | | | | | 6.65 (dd, 1H), 6.83 (d, 2H), |
| | | | | | 6.96 (ddd, 1H), 7.15 (dd, |
| | | | | | 1H), 7.17 (d, 2H), |
| | | | | | 7.30 (dd, 1H), 8.37 (d, 1H) |
| 40.65 | 2-bromo-4- | N-[(2-bromo-4- | 60 | 517 | 2.74 (d, 3H), 2.91 (dd, |
| | fluorobenzoic | fluorophenyl)carbonyl]- | | | 1H) 2.94 (t, 2H), 3.11 (dd, |
| | acid | O-{2-[6- | | | 1H), 4.25 (t, 2H), |
| | | (methylamino)pyridin- | | | 4.36 (ddd, 1H), 6.27 (d, 1H), |
| | | 2-yl]ethyl}-L- | | | 6.35 (q, 1H), 6.43 (d, 1H), |
| | | tyrosine | | | 6.83 (d, 2H), 7.16 (d, 2H), |
| | | | | | 7.26-7.34 (m, 3H), |
| | | | | | 7.59 (dd, 1H), 8.26 (bs, 1H) |
| 40.66 | 2-bromo- | N-[(2-bromo-4,5- | 66 | 535 | 2.74 (d, 3H), 2.89 (dd, |
| | 4,5- | difluorophenyl)carbonyl]- | | | 1H), 2.94 (t, 2H), 3.10 (dd, |
| | difluorobenzoic | O-{2-[6- | | | 1H), 4.26 (t, 2H), |
| | acid | (methylamino)pyridin- | | | 4.37-4.44 (m, 1H), 6.27 (d, 1H), |
| | | 2-yl]ethyl}-L- | | | 6.35 (q, 1H), 6.43 (d, 1H), |
| | | tyrosine | | | 6.84 (d, 2H), 7.16 (d, 2H), |
| | | | | | 7.24 (dd, 1H), 7.30 (dd, |
| | | | | | 1H), 7.88 (dd, 1H), |
| | | | | | 8.49 (bs, 1H) |
| 40.67 | 2-bromo-5- | N-[(2-bromo-5- | 65 | 517 | 2.74 (d, 3H), 2.90 (dd, |
| | fluorobenzoic | fluorophenyl)carbonyl]- | | | 1H), 2.94 (t, 2H), 3.11 (dd, |
| | acid | O-{2-[6- | | | 1H), 4.26 (t, 2H), |
| | | (methylamino)pyridin- | | | 4.39-4.47 (m, 1H), 6.27 (d, 1H), |
| | | 2-yl]ethyl}-L- | | | 6.35 (q, 1H), 6.43 (d, 1H), |
| | | tyrosine | | | 6.85 (d, 2H), 6.99 (dd, |
| | | | | | 1H), 7.17 (d, 2H), |
| | | | | | 7.25 (dd, 1H), 7.30 (dd, 1H), |
| | | | | | 7.66 (dd, 1H), 8.54 (bs, |
| | | | | | 1H) |

The starting material methyl O-{2-[6-(methylamino)pyridin-2-yl]ethyl}-L-tyrosinate used in Example 40 was prepared as follows:

To a solution of methyl N-(tert-butoxycarbonyl)-L-tyrosinate (6.5 g, 22.0 mmol) in DCM (100 mL) at −30° C. were added triphenylphosphine (12.12 g, 46.2 mmol) and tert-Butyl [6-(2-hydroxyethyl)pyridin-2-yl]methylcarbamate (6.11 g, 24.2 mmol). ADDP (11.66 g, 46.2 mmol) in DCM (50 ml) was then added dropwise over 10 minutes. The reaction mixture was allowed to stir at room temperature 18 hours, and was then concentrated and partially purified by silica gel flash chromatography (20 to 40% ethyl acetate in petroleum ether) to give a colorless oil contaminated by triphenylphosphine oxide. This oil was stirred in HCl 4N in 1,4-dioxane (100 ml) for 1 hour at room temperature. Evaporation was followed by dissolution in DCM and slow addition of a solution of NH₃ 7N in MeOH at 0° C. removal of salts by filtration was followed by a purification by silica gel flash chromatography (1 to 3% NH₃/MeOH 7N in DCM) to give methyl O-{2-[6-(methylamino)pyridin-2-yl]ethyl}-L-tyrosinate as a gum (4.09 g, 56%); ¹H NMR Spectrum (CDCl3) 1.67 (bs, 2H), 2.80 (dd, 1H), 2.90 (d, 3H), 3.01 (dd, 1H), 3.07 (t, 2H), 3.68 (dd, 1H), 3.71 (s, 3H), 4.30 (t, 2H), 4.71 (bs, 1H), 6.26 (d, 1H), 6.55 (d, 1H), 6.85 (d, 2H), 7.08 (d, 2H), 7.40 (dd, 1H).

### Example 41

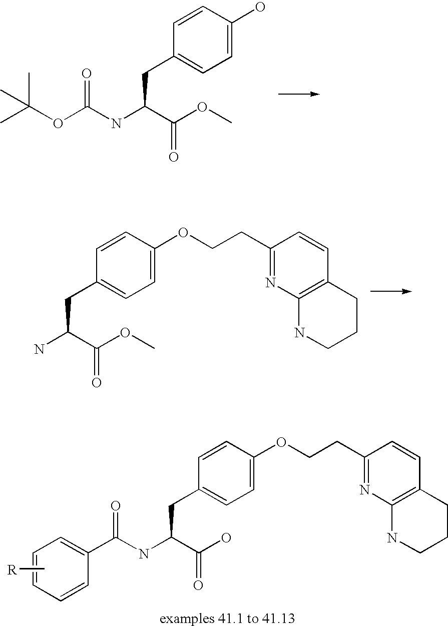

To 2,4-dichlorobenzoic acid (71 mg, 0.37 mmol) and TBTU (141 mg, 0.37 mmol) was added a solution of methyl O-[2-(5,6,7,8-tetrahydro-1,8-naphthyridin-2-yl)ethyl]-L-tyrosinate (120 mg, 0.34 mmol) in DMF (1 ml). The reaction mixture was stirred at room temperature for 72 hours. Then LiOH (62 mg, 1.35 mmol) and water (0.2 ml) were added, and the mixture was stirred 24 hours. A filtration was directly followed by a purification by C18 reverse phase chromatography (basic conditions) to afford example 41.1 (N-[(2,4-dichlorophenyl)carbonyl]-O-[2-(5,6,7,8-tetrahydro-1,8-naphthyridin-2-yl)ethyl]-L-tyrosine) (Example 41.1) as a solid.

The procedure described above for example 41.1 was repeated by coupling methyl O-[2-(5,6,7,8-tetrahydro-1,8-naphthyridin-2-yl)ethyl]-L-tyrosinate with the appropriate benzoic acid. Thus were obtained the compounds shown in Table 10:

TABLE 10

| | | | | Mass | ¹H NMR Data (500 MHz - |
|---|---|---|---|---|---|
| Example | R | Name | Yield | Ion | DMSO d6) |
| 41.1 | 2,4- | N-[(2,4- | 42% | 514 [M + H]⁺ | 1.71-1.78 (m, 2H), |
| | dichloro | dichlorophenyl) | | | 2.61 (t, 2H), |
| | | carbonyl]- | | | 2.83-2.91 (m, 3H), 3.09 (dd, |
| | | O-[2- | | | 1H), 3.21-3.26 (m, |
| | | (5,6,7,8- | | | 2H), 4.19 (t, 2H), |
| | | tetrahydro- | | | 4.49 (ddd, 1H), 6.36 (d, |
| | | 1,8- | | | 1H), 6.40 (bs, 1H), |
| | | naphthyridin- | | | 6.82 (d, 2H), 7.06 (d, |
| | | 2-yl)ethyl]-L- | | | 1H), 7.17 (d, 2H), |
| | | tyrosine | | | 7.26 (d, 1H), 7.47 (dd, 1H), |
| | | | | | 7.63 (d, 1H), 8.67 (d, |
| | | | | | 1H) |
| 41.2 | 2-chloro- | N-[(2-chloro- | 71% | 516 [M + H]⁺ | 1.71-1.78 (m, 2H), |
| | 3,6- | 3,6- | | | 2.61 (t, 2H), |
| | difluoro | difluorophenyl) | | | 2.82-2.90 (m, 3H), 3.07 (dd, |
| | | carbonyl]- | | | 1H), 3.21-3.26 (m, |
| | | O-[2- | | | 2H), 4.19 (t, 2H), |
| | | (5,6,7,8- | | | 4.50 (ddd, 1H), 6.36 (d, |
| | | tetrahydro- | | | 1H), 6.38 (bs, 1H), |
| | | 1,8- | | | 6.80 (d, 2H), 7.06 (d, |
| | | naphthyridin- | | | 1H), 7.15 (d, 2H), |
| | | 2-yl)ethyl]-L- | | | 7.32 (ddd, 1H), 7.50 (ddd, |
| | | tyrosine | | | 1H), 8.89 (d, 1H) |
| 41.3 | 2-chloro- | N-[(2-chloro- | 68% | 494 [M + H]⁺ | 1.71-1.78 (m, 2H), |
| | 5-methyl | 5- | | | 2.28 (s, 3H), 2.61 (t, |
| | | methylphenyl) | | | 2H), 2.83-2.92 (m, |
| | | carbonyl]-O- | | | 3H), 3.08 (dd, 1H), |
| | | [2-(5,6,7,8- | | | 3.20-3.26 (m, 2H), |
| | | tetrahydro- | | | 4.19 (t, 2H), |
| | | 1,8- | | | 4.50 (ddd, 1H), 6.35 (d, |
| | | naphthyridin- | | | 1H), 6.36 (bs, 1H), |
| | | 2-yl)ethyl]-L- | | | 6.83 (d, 2H), 7.02 (d, |
| | | tyrosine | | | 1H), 7.05 (d, 1H), |
| | | | | | 7.18 (d, 2H), 7.23 (dd, 1H), |
| | | | | | 7.32 (d, 1H), 8.57 (d, |
| | | | | | 1H) |
| 41.4 | 2,4,6- | O-[2- | 52% | 488 [M + H]⁺ | 1.70-1.79 (m, 2H), |
| | trimethyl | (5,6,7,8- | | | 1.94 (s, 3H), 2.19 (s, |
| | | tetrahydro- | | | 6H), 2.62 (t, 2H), |
| | | 1,8- | | | 2.79 (dd, 1H), 2.86 (t, 2H), |
| | | naphthyridin- | | | 3.10 (dd, 1H), |
| | | 2-yl)ethyl]- | | | 3.21-3.29 (m, 2H), 4.19 (t, |
| | | N-[(2,4,6- | | | 2H), 4.57 (ddd, 1H), |
| | | trimethylphenyl) | | | 6.35 (d, 1H), 6.38 (bs, |
| | | carbonyl]- | | | 1H), 6.76 (s, 2H), |
| | | L-tyrosine | | | 6.81 (d, 2H), 7.06 (d, 1H), |
| | | | | | 7.17 (d, 2H), 8.30 (d, |
| | | | | | 1H) |
| 41.5 | 2-chloro- | N-[(2-chloro- | 65% | 494 [M + H]⁺ | 1.70-1.79 (m, 2H), |
| | 6-methyl | 6- | | | 2.07 (s, 3H), 2.61 (t, |
| | | methylphenyl) | | | 2H), 2.84 (dd, 1H), |
| | | carbonyl]-O- | | | 2.86 (t, 2H), 3.08 (dd, |
| | | [2-(5,6,7,8- | | | 1H), 3.21-3.26 (m, |
| | | tetrahydro- | | | 2H), 4.19 (t, 2H), |
| | | 1,8- | | | 4.55 (ddd, 1H), 6.36 (d, |
| | | naphthyridin- | | | 1H), 6.38 (bs, 1H), |
| | | 2-yl)ethyl]-L- | | | 6.81 (d, 2H), 7.06 (d, |
| | | tyrosine | | | 1H), 7.14 (dd, 1H), |
| | | | | | 7.18 (d, 2H), |
| | | | | | 7.20-7.27 (m, 2H), 8.63 (d, |
| | | | | | 1H) |
| 41.6 | 2-chloro- | N-[(2-chloro- | 76% | 498 [M + H]⁺ | 1.71-1.79 (m, 2H), |
| | 4-fluoro | 4- | | | 2.61 (t, 2H), |
| | | fluorophenyl) | | | 2.83-2.92 (m, 3H), 3.10 (dd, |
| | | carbonyl]-O- | | | 1H), 3.20-3.26 (m, |
| | | [2-(5,6,7,8- | | | 2H), 4.18 (t, 2H), |
| | | tetrahydro- | | | 4.44 (ddd, 1H), 6.35 (d, |
| | | 1,8- | | | 1H), 6.38 (bs, 1H), |
| | | naphthyridin- | | | 6.81 (d, 2H), 7.05 (d, |
| | | 2-yl)ethyl]-L- | | | 1H), 7.16 (d, 2H), |
| | | tyrosine | | | 7.25 (ddd, 1H), 7.32 (dd, |
| | | | | | 1H), 7.45 (dd, 1H), |
| | | | | | 8.45 (bs, 1H) |
| 41.7 | 2-chloro- | N-[(2-chloro- | 74% | 498 [M + H]⁺ | 1.70-1.79 (m, 2H), |
| | 6-fluoro | 6- | | | 2.61 (t, 2H), |
| | | fluorophenyl) | | | 2.82-2.92 (m, 3H), 3.06 (dd, |
| | | carbonyl]-O- | | | 1H), 3.20-3.26 (m, |
| | | [2-(5,6,7,8- | | | 2H), 4.18 (t, 2H), |
| | | tetrahydro- | | | 4.46 (ddd, 1H), 6.35 (d, |
| | | 1,8- | | | 1H), 6.38 (bs, 1H), |
| | | naphthyridin- | | | 6.79 (d, 2H), 7.05 (d, |
| | | 2-yl)ethyl]-L- | | | 1H), 7.15 (d, 2H), |
| | | tyrosine | | | 7.24 (dd, 1H), 7.30 (d, 1H), |
| | | | | | 7.43 (ddd, 1H), |
| | | | | | 8.70 (bs, 1H) |
| 41.8 | 2,6- | N-[(2,6- | 64% | 474 [M + H]⁺ | 1.71-1.79 (m, 2H), |
| | dimethyl | dimethylphenyl) | | | 1.98 (s, 6H), 2.61 (t, |
| | | carbonyl]- | | | 2H), 2.79 (dd, 1H), |
| | | O-[2- | | | 2.86 (t, 2H), 3.11 (dd, |
| | | (5,6,7,8- | | | 1H), 3.21-3.27 (m, |
| | | tetrahydro- | | | 2H), 4.19 (t, 2H), |
| | | 1,8- | | | 4.59 (ddd, 1H), 6.35 (d, |
| | | naphthyridin- | | | 1H), 6.39 (bs, 1H), |
| | | 2-yl)ethyl]-L- | | | 6.82 (d, 2H), 7.94 (d, |
| | | tyrosine | | | 2H), 7.06 (d, 1H), |
| | | | | | 7.10 (t, 1H), 7.19 (d, 2H), |
| | | | | | 8.40 (d, 1H) |
| 41.9 | 6-chloro- | N-[(6-chloro- | 61% | 512 [M + H]⁺ | 1.71-1.79 (m, 2H), |
| | 2-fluoro- | 2-fluoro-3- | | | 2.20 (s, 3H), 2.61 (t, |
| | 3-methyl | methylphenyl) | | | 2H), 2.86 (t, 2H), |
| | | carbonyl]-O- | | | 2.89 (dd, 1H), 3.06 (dd, |
| | | [2-(5,6,7,8- | | | 1H), 3.21-3.26 (m, |
| | | tetrahydro- | | | 2H), 4.18 (t, 2H), |
| | | 1,8- | | | 4.41 (ddd, 1H), 6.35 (d, |
| | | naphthyridin- | | | 1H), 6.38 (bs, 1H), |
| | | 2-yl)ethyl]-L- | | | 7.78 (d, 2H), 7.05 (d, |
| | | tyrosine | | | 1H), 7.15 (d, 2H), |
| | | | | | 7.18 (d, 1H), 7.30 (dd, 1H), |
| | | | | | 8.46 (bs, 1H) |
| 41.10 | 2-chloro | N-[(2- | 72% | 481 [M + H]⁺ | 1.71-1.78 (m, 2H), |
| | | chlorophenyl) | | | 2.61 (t, 2H), 2.86 (t, |
| | | carbonyl]-O- | | | 2H), 2.89 (dd, 1H), |
| | | [2-(5,6,7,8- | | | 3.09 (dd, 1H), |
| | | tetrahydro- | | | 3.20-3.26 (m, 2H), 4.19 (t, |
| | | 1,8- | | | 2H), 4.48 (ddd, 1H), |
| | | naphthyridin- | | | 6.35 (d, 1H), 6.36 (bs, |
| | | 2-yl)ethyl]-L- | | | 1H), 6.83 (d, 2H), |
| | | tyrosine | | | 7.05 (d, 1H), 7.18 (d, 2H), |
| | | | | | 7.25 (dd, 1H), |
| | | | | | 7.36 (ddd, 1H), 7.42 (ddd, |
| | | | | | 1H), 7.45 (dd, 1H), |
| | | | | | 8.57 (d, 1H) |
| 41.11 | 5-chloro- | N-[(5-chloro- | 74% | 524 [M + H]⁺ | 1.70-1.79 (m, 2H), |
| | 1,3- | 1,3- | | | 2.60 (t, 2H), 2.85 (t, |
| | benzodioxol- | benzodioxol- | | | 2H), 2.95 (dd, 1H), |
| | 4-yl | 4- | | | 3.07 (dd, 1H), |
| | | yl)carbonyl]- | | | 3.19-3.26 (m, 2H), |
| | | O-[2- | | | 4.11-4.20 (m, 3H), 6.08 (d, |
| | | (5,6,7,8- | | | 2H), 6.33 (bs, 1H), |
| | | tetrahydro- | | | 6.35 (d, 1H), 6.72 (d, |
| | | 1,8- | | | 2H), 6.89 (d, 1H), |
| | | naphthyridin- | | | 6.92 (d, 1H), 7.04 (d, 1H), |
| | | 2-yl)ethyl]-L- | | | 7.10 (d, 2H), 7.62 (bs, |
| | | tyrosine | | | 1H) |
| 41.12 | 2-methyl | N-[(2- | 62% | 460 [M + H]⁺ | 1.70-1.79 (m, 2H), |
| | | methylphenyl) | | | 2.17 (s, 3H), 2.61 (t, |
| | | carbonyl]-O- | | | 2H), 2.86 (t, 2H), |
| | | [2-(5,6,7,8- | | | 2.88 (dd, 1H), 3.11 (dd, |
| | | tetrahydro- | | | 1H), 3.20-3.26 (m, |
| | | 1,8- | | | 2H), 4.19 (t, 2H), |
| | | naphthyridin- | | | 4.47 (ddd, 1H), 6.35 (d, |
| | | 2-yl)ethyl]-L- | | | 1H), 6.39 (bs, 1H), |
| | | tyrosine | | | 6.82 (d, 2H), 7.05 (d, |
| | | | | | 1H), 7.14-7.21 (m, |
| | | | | | 5H), 7.28 (ddd, 1H), |
| | | | | | 8.21 (d, 1H) |
| 41.13 | 2-fluoro- | N-[(2-fluoro- | 62% | 478 [M + H]⁺ | 1.70-1.78 (m, 2H), |
| | 6-methyl | 6- | | | 2.34 (s, 3H), 2.60 (t, |
| | | methylphenyl) | | | 2H), 2.85 (t, 2H), |
| | | carbonyl]-O- | | | 2.97 (dd, 1H), 3.10 (dd, |
| | | [2-(5,6,7,8- | | | 1H), 3.20-3.25 (m, |
| | | tetrahydro- | | | 2H), 4.17 (t, 2H), |
| | | 1,8- | | | 4.39-4.46 (m, 1H), |
| | | naphthyridin- | | | 6.35 (d, 1H), 6.37 (bs, |
| | | 2-yl)ethyl]-L- | | | 1H), 6.78 (d, 2H), |
| | | tyrosine | | | 7.05 (d, 1H), 7.06-7.13 (m, |
| | | | | | 4H), 7.53 (dd, 1H), |
| | | | | | 8.05-8.13 (m, 1H) |

The starting material methyl O-[2-(5,6,7,8-tetrahydro-1,8-naphthyridin-2-yl)ethyl]-L-tyrosinate used in Example 41 was prepared as follows:

To a solution of methyl N-(tert-butoxycarbonyl)-L-tyrosinate (6.5 g, 22.0 mmol) in DCM (100 mL) at −30° C. were added triphenylphosphine (12.12 g, 46.2 mmol) and 2-(5,6,7,8-tetrahydro-1,8-naphthyridin-2-yl)ethanol (WO 2004/058761 page 34) (4.31 g, 24.2 mmol). ADDP (11.66 g, 46.2 mmol) in DCM (50 ml) was then added dropwise over 10 minutes. The reaction mixture was allowed to stir at room temperature 18 hours, and was then concentrated and partially purified by silica gel flash chromatography (20 to 40% ethyl acetate in petroleum ether) to give a yellow solid contaminated by triphenylphosphine oxide. This solid was stirred in TFA (100 ml) for 2 hours at room temperature. Evaporation was followed by dissolution in DCM and slow addition of a solution of NH₃ 7N in MeOH at 0° C. Removal of salts by filtration was followed by a purification by silica gel flash chromatography (1 to 4% NH₃/MeOH 7N in DCM) to give methyl O-[2-(5,6,7,8-tetrahydro-1,8-naphthyridin-2-yl)ethyl]-L-tyrosinate as a yellow solid (6.17 g, 79%); ¹H NMR Spectrum (CDCl3) 1.86-1.94 (m, 2H), 2.70 (t, 2H), 2.97-3.04 (m, 3H), 3.37-3.43 (m, 2H), 3.68 (dd, 1H), 3.71 (s, 3H), 4.24 (t, 2H), 4.84 (bs, 1H), 6.45 (d, 1H), 6.84 (d, 2H), 7.07 (d, 2H), 7.08 (d, 1H).

### Example 42.1

### N-[(2-chlorophenyl)carbonyl]-3-(5-{3-[6-(methylamino)pyridin-2-yl]propyl}pyridin-2-yl)-L-alanine

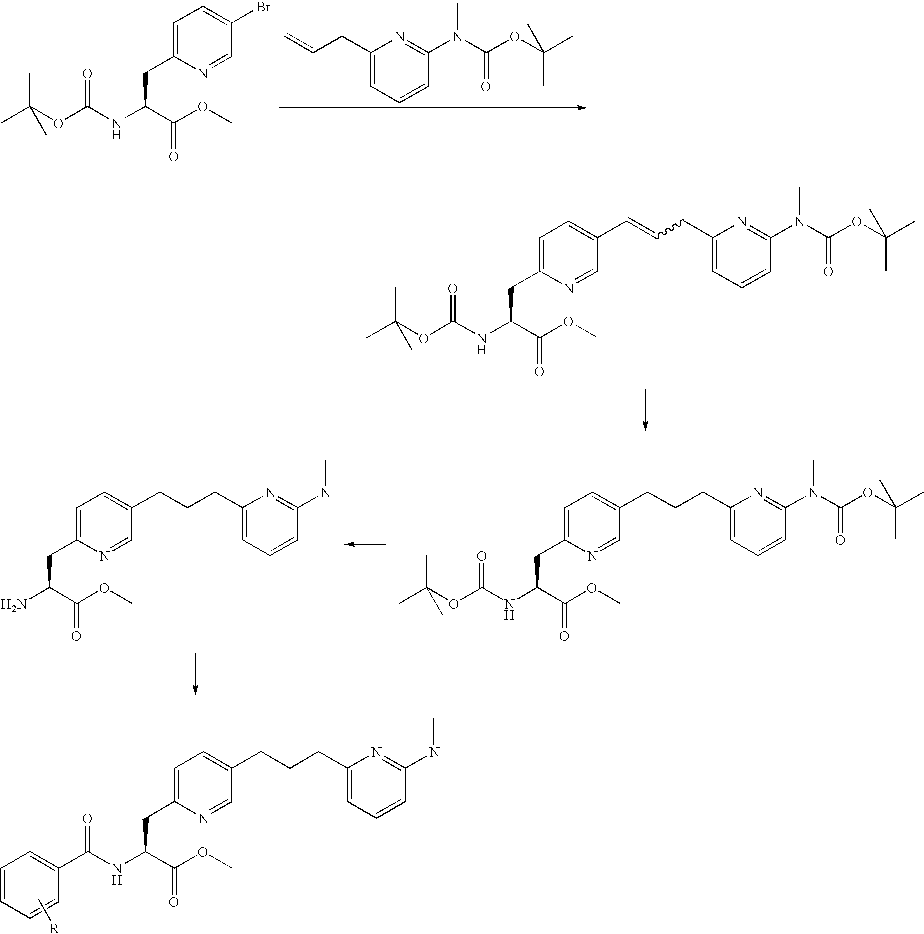

To a solution of methyl 3-(5-{3-[6-(methylamino)pyridin-2-yl]propyl}pyridin-2-yl)-L-alaninate (200 mg, 0.46 mmol), 2-chlorobenzoic acid (108 mg, 0.69 mmol) and N-methylmorpholine (0.2 ml, 2.00 mmol) in DMF (1.5 ml) was added TBTU (262 mg, 0.69 mmol). The reaction mixture was stirred at room temperature for 16 hours. Then LiOH (84 mg, 1.83 mmol) was added, and the mixture was stirred for 4 hours. A filtration was directly followed by two purifications by C18 reverse phase chromatography (basic conditions then acidic conditions) to afford the title compound as a solid (60 mg, 26%).

The procedure described above for example 42.1 was repeated by coupling methyl 3-(5-{3-[6-(methylamino)pyridin-2-yl]propyl}pyridin-2-yl)-L-alaninate with the appropriate benzoic acid. Thus were obtained the compounds shown in Table 11:

TABLE 11

| | | | | | ¹H NMR |
|---|---|---|---|---|---|
| | | | | Mass | Data (500 |
| Example | R | Name | Yield | Ion | MHz) |
| 42.1 | 2- | N-[(2- | 26% | 453 | 1.86-1.97(m, |
| | chloro | chlorophenyl)carbonyl]- | | [M + H]⁺ | 2H), 2.52(t |
| | | 3-(5-{3-[6- | | | partially |
| | | (methylamino)pyridin-2- | | | hidden by |
| | | yl]propyl}pyridin-2-yl)- | | | DMSOd5, |
| | | L-alanine | | | 2H), 2.59(t, |
| | | | | | 2H), 2.74(d, |
| | | | | | 3H), 3.08(dd, |
| | | | | | 1H), 3.26(dd |
| | | | | | partially |
| | | | | | hidden by |
| | | | | | H2O, 1H), |
| | | | | | 4.70(bs, 1H) |
| | | | | | 6.22(d, 1H), |
| | | | | | 6.29(q, 1H), |
| | | | | | 6.32(d, 1H), |
| | | | | | 7.22(d, 1H), |
| | | | | | 7.25-7.30(m, |
| | | | | | 2H), 7.34 |
| | | | | | (ddd, 1H), |
| | | | | | 7.38-7.44(m, |
| | | | | | 2H), 7.54(dd, |
| | | | | | 1H), 8.34(s, |
| | | | | | 1H), 8.46(bs, |
| | | | | | 1H) |
| 42.2 | 2,6- | N-[(2,6- | 36% | 487 | 1.86-1.95(m, |
| | dichloro | dichlorophenyl)carbonyl]- | | [M + H]⁺ | 2H), 2.52(t |
| | | 3-(5-{3-[6- | | | partially |
| | | (methylamino)pyridin-2- | | | hidden by |
| | | yl]propyl}pyridin-2-yl)- | | | DMSOd5, |
| | | L-alanine | | | 2H), 2.58(t, |
| | | | | | 2H), 2.74(d, |
| | | | | | 3H), 3.04(dd, |
| | | | | | 1H), 3.24(dd |
| | | | | | partially |
| | | | | | hidden by |
| | | | | | H2O, 1H), |
| | | | | | 4.77(bs, 1H), |
| | | | | | 6.22(d, 1H), |
| | | | | | 6.30(q, 1H), |
| | | | | | 6.33(d, 1H), |
| | | | | | 7.22(d, 1H), |
| | | | | | 7.28(dd, 1H), |
| | | | | | 7.36(dd, 1H), |
| | | | | | 7.38-7.42(m, |
| | | | | | 2H), 7.52(dd, |
| | | | | | 1H), 8.33(s, |
| | | | | | 1H), 8.71(bs, |
| | | | | | 1H) |

The methyl 3-(5-{3-[6-(methylamino)pyridin-2-yl]propyl}pyridin-2-yl)-L-alaninate used as the starting material was prepared as follows:

A solution of tert-butyl (6-allylpyridin-2-yl)methylcarbamate (630 mg, 2.54 mmol) (described in example 18), methyl (2S)-3-(5-bromopyridin-2-yl)-2-[(2-methylpropan-2-yl)oxycarbonylamino]propanoate (912 mg, 2.54 mmol) (Organic and Biomolecular Chemistry 2003, 1 (23), 4254), tri-O-tolylphosphine (307 mg, 1.00 mmol), Palladium(II) acetate (114 mg, 0.51 mmol) and DIPEA (0.57 ml, 3.30 mmol) in acetonitrile (4 ml) was degassed, then sealed and heated to 150° C. for 20 minutes in microwave. After concentration, a purification by silica gel flash chromatography (10 to 50% ethyl acetate in petroleum ether) afforded methyl N-(tert-butoxycarbonyl)-3-{5-[3-{6-[(tert-butoxycarbonyl)(methyl)amino]pyridin-2-yl}prop-1-en-1-yl]pyridin-2-yl}-L-alaninate as a cis-trans mixture (968 mg, 72%); Mass Spectrum [M+H]⁺=527.

A mixture of methyl N-(tert-butoxycarbonyl)-3-{5-[3-{6-[(tert-butoxycarbonyl)(methyl)amino]pyridin-2-yl}prop-1-en-1-yl]pyridin-2-yl}-L-alaninate (965 mg, 1.83 mmol) and Pd/C 10% (400 mg) in ethanol (100 ml) was hydrogenated under 1.5 bar of H₂ for 16 hours, filtered through celite, evaporated and purified by C18 reverse phase chromatography (acidic conditions) to afford methyl N-(tert-butoxycarbonyl)-3-[5-(3-{6-[(tert-butoxycarbonyl)(methyl)amino]pyridin-2-yl}propyl)pyridin-2-yl]-L-alaninate as a clear colourless oil (600 mg, 62%); Mass Spectrum [M+H]⁺=529; ¹H NMR Spectrum (DMSO-d6) 1.31 (s, 9H), 1.44 (s, 9H), 1.92-2.01 (m, 2H), 2.61 (t, 2H), 2.69 (t, 2H), 2.97 (dd, 1H), 3.08 (dd, 1H), 3.27 (s, 3H), 3.59 (s, 3H), 4.42 (ddd, 1H), 6.98 (d, 1H), 7.17 (d, 1H), 7.24 (d, 1H), 7.40 (d, 1H), 7.56 (d, 1H), 7.65 (dd, 1H), 8.35 (s, 1H).

Methyl N-(tert-butoxycarbonyl)-3-[5-(3-{6-[(tert-butoxycarbonyl)(methyl)amino]pyridin-2-yl}propyl)pyridin-2-yl]-L-alaninate (600 mg, 1.14 mmol) in methanol (10 ml) was stirred with a solution of HCl 4M in dioxane (5 ml) at room temperature overnight. Evaporation was followed by trituration in DCM/Et₂O to obtain a precipitate, which was collected by filtration and dried to a constant weight to afford methyl 3-(5-{3-[6-(methylamino)pyridin-2-yl]propyl}pyridin-2-yl)-L-alaninate tri hydrochloride as a beige foam (408 mg, 82%); Mass Spectrum [M+H]⁺=329.

### Example 43

### N-[(2-chlorophenyl)carbonyl]-4-{4-[6-(methylamino)pyridin-2-yl]butyl}-L-phenylalanine

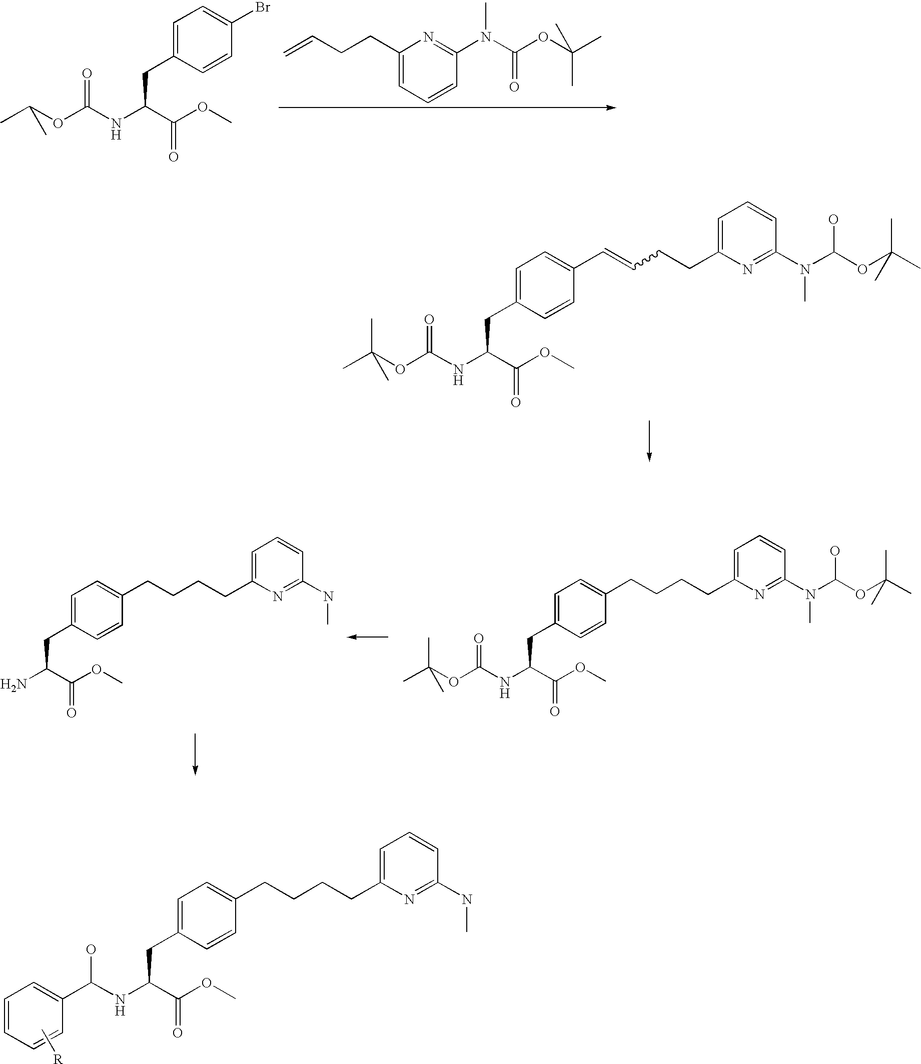

To a solution of methyl 4-{4-[6-(methylamino)pyridin-2-yl]butyl}-L-phenylalaninate (300 mg, 0.69 mmol), 2-chlorobenzoic acid (161 mg, 1.03 mmol) and N-methylmorpholine (0.33 ml, 3.02 mmol) in DMF (1.8 ml) was added TBTU (392 mg, 1.03 mmol). The reaction mixture was stirred at room temperature for 16 hours. Then LiOH (127 mg, 2.75 mmol) was added, and the mixture was stirred for 4 hours. A filtration was directly followed by two purifications by C18 reverse phase chromatography (basic conditions then acidic conditions) to afford the title compound as a solid (1187 mg, 58%).

The procedure described above for example 43.1 was repeated by coupling methyl 4-{4-[6-(methylamino)pyridin-2-yl]butyl}-L-phenylalaninate with the appropriate benzoic acid. Thus were obtained the compounds shown in Table 12:

TABLE 12

| | | | | Mass | ¹H NMR Data (500 MHz - |
|---|---|---|---|---|---|
| Example | R | Name | Yield | Ion | DMSA d6) |
| 43.1 | 2- | N-[(2- | 58% | 466 [M + H]⁺ | 1.52-1.66 (m, 4H), |
| | chloro | chlorophenyl)carbonyl]- | | | 2.51 (t partially hidden |
| | | 4-{4-[6- | | | by DMSOd5, 2H), |
| | | (methylamino)pyridin- | | | 2.56 (t, 2H), 2.72 (d, 3H), |
| | | 2-yl]butyl}-L- | | | 2.91 (dd, 1H), 3.13 (dd, |
| | | phenylalanine | | | 1H), 4.52 (bs, 1H), |
| | | | | | 6.20 (d, 1H), 6.25 (q, |
| | | | | | 1H), 6.30 (d, 1H), |
| | | | | | 7.09 (d, 2H), 7.13-7.22 (m, |
| | | | | | 3H), 7.25 (dd, 1H), |
| | | | | | 7.32 (ddd, 1H), |
| | | | | | 7.38-7.46 (m, 2H), 8.61 (bs, |
| | | | | | 1H) |
| 43.2 | 2,6- | N-[(2,6- | 33% | 500 [M + H]⁺ | 1.51-1.66 (m, 4H), |
| | dichloro | dichlorophenyl)carbonyl]- | | | 2.52 (t partially hidden |
| | | 4-{4-[6- | | | by DMSOd5, 2H), |
| | | (methylamino)pyridin- | | | 2.55 (t, 2H), 2.72 (d, 3H), |
| | | 2-yl]butyl}-L- | | | 2.88 (dd, 1H), 3.09 (dd, |
| | | phenylalanine | | | 1H), 4.61 (ddd, 1H), |
| | | | | | 6.20 (d, 1H), 6.26 (q, |
| | | | | | 1H), 6.31 (d, 1H), |
| | | | | | 7.07 (d, 2H), 7.18 (d, 2H), |
| | | | | | 7.26 (dd, 1H), 7.38 (dd, |
| | | | | | 1H), 7.40-7.40 (m, |
| | | | | | 2H), 8.95 (bs, 1H) |

The methyl 4-{4-[6-(methylamino)pyridin-2-yl]butyl}-L-phenylalaninate used as the starting material was prepared as follows:

A solution of tert-butyl (6-but-3-en-1-ylpyridin-2-yl)methylcarbamate (800 mg, 3.20 mmol) (described in example 5), methyl 4-bromo-N-(tert-butoxycarbonyl)-L-phenylalaninate (1148 mg, 3.20 mmol) (as described in example 18), tri-O-tolylphosphine (390 mg, 1.28 mmol), Palladium(II) acetate (142 mg, 0.64 mmol) and DIPEA (0.73 ml, 4.17 mmol) in acetonitrile (6 ml) was degassed, then sealed and heated to 150° C. for 20 minutes in microwave. After concentration, a purification by silica gel flash chromatography (20 ethyl acetate in petroleum ether) afforded methyl N-(tert-butoxycarbonyl)-4-[4-{6-[(tert-butoxycarbonyl)(methyl)amino]pyridin-2-yl}but-1-en-1-yl]-L-phenylalaninate as a cis-trans mixture (1.40 g, 81%); Mass Spectrum [M+H]⁺=540.

A mixture of methyl N-(tert-butoxycarbonyl)-4-[4-{6-[(tert-butoxycarbonyl)(methyl)amino]pyridin-2-yl}but-1-en-1-yl]-L-phenylalaninate (1.40 g, 2.66 mmol) and Pd/C 10% (1.00 g) in ethanol (100 ml) was hydrogenated under 1.5 bar of H₂ for 16 hours, filtered through celite, evaporated and purified by C18 reverse phase chromatography (acidic conditions) to afford methyl N-(tert-butoxycarbonyl)-4-(4-{6-[(tert-butoxycarbonyl)(methyl)amino]pyridin-2-yl}butyl)-L-phenylalaninate as a clear colourless oil (1.02 g, 71%); Mass Spectrum [M+H]⁺=542; ¹H NMR Spectrum (DMSO-d6) 1.30 (s, 9H), 1.45 (s, 9H), 1.52-1.61 (m, 2H), 1.61-1.70 (m, 2H), 2.56 (t, 2H), 2.68 (t, 2H), 2.79 (dd, 1H), 2.93 (dd, 1H), 3.25 (s, 3H), 3.59 (s, 3H), 4.14 (ddd, 1H), 6.95 (d, 1H), 7.05-7.17 (m, 4H), 7.26 (d, 1H), 7.38 (d, 1H), 7.63 (dd, 1H).

Methyl N-(tert-butoxycarbonyl)-4-(4-{6-[(tert-butoxycarbonyl)(methyl)amino]pyridin-2-yl}butyl)-L-phenylalaninate (900 mg, 1.70 mmol) in methanol (5 ml) was stirred with a solution of HCl 4M in dioxane (10 ml) at room temperature overnight. Evaporation was followed by trituration in DCM/Et₂O to obtain a precipitate, which was collected by filtration and dried to a constant weight to afford methyl 4-{4-[6-(methylamino)pyridin-2-yl]butyl}-L-phenylalaninate tri hydrochloride as a brown foam (614 mg, 82%); Mass Spectrum [M+H]⁺=342.

### Example 44

### N-[(2,6-dichlorophenyl)carbonyl]-O-[2-(5-methyl-5,6,7,8-tetrahydro-1,8-naphthyridin-2-yl)ethyl]-L-tyrosine

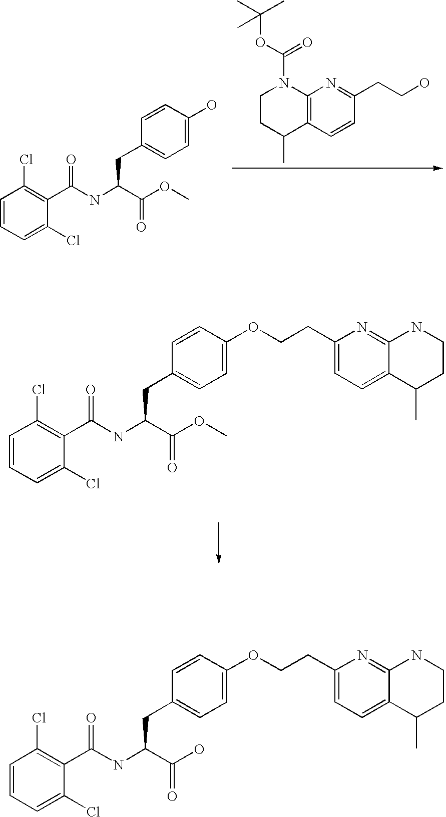

To a solution of methyl N-[(2,6-dichlorophenyl)carbonyl]-O-[2-(5-methyl-5,6,7,8-tetrahydro-1,8-naphthyridin-2-yl)ethyl]-L-tyrosinate (250 mg, 0.46 mmol) in DMF (1.5 ml) were added water (0.3 ml) and LiOH (85 mg, 1.84 mmol). After 18 hours at room temperature, the reaction mixture was filtered and purified by C18 reverse phase chromatography (acidic conditions) to afford the title compound as a white solid (100 mg, 41%); Mass Spectrum [M+H]⁺=528; ¹H NMR Spectrum (DMSO-d6) 1.18 (d, 3H), 1.46-1.54 (m, 1H), 1.75-1.83 (m, 1H), 2.74-2.82 (m, 1H), 2.83-2.91 (m, 3H), 3.04 (dd, 1H), 3.21-3.28 (m, 2H), 4.19 (t, 2H), 4.54 (bs, 1H), 6.38 (bs, 1H), 6.39 (d, 1H), 6.81 (d, 2H), 7.14-7.20 (m, 3H), 7.39 (dd, 1H), 7.42-7.46 (m, 2H), 8.90 (bs, 1H)

The starting material methyl N-[(2,6-dichlorophenyl)carbonyl]-O-[2-(5-methyl-5,6,7,8-tetrahydro-1,8-naphthyridin-2-yl)ethyl]-L-tyrosinate was prepared as follows:

To a solution of tert-butyl 7-(2-hydroxyethyl)-4-methyl-3,4-dihydro-1,8-naphthyridine-1(2H)-carboxylate (272 mg, 0.93 mmol), methyl N-(2,6-dichlorobenzoyl)-L-tyrosinate (310 mg, 0.84 mmol) and triphenylphosphine (330 mg, 1.26 mmol) in DCM (2 ml) was slowly added a solution of ADDP (318 mg, 1.26 mmol) in DCM (2 ml). The reaction mixture was stirred overnight, then solvent was evaporated and the residue was purified by silica gel flash chromatography (20 to 60% ethyl acetate in petroleum ether) to afford a white foam, which was immediately diluted in DCM (2 ml). TFA (4 ml) was added and the mixture stirred overnight. Solvent were evaporated to dryness, then the residue was diluted in DCM and extracted with aqueous NaOH 2N. The organic layer was dried and evaporated to afford methyl N-[(2,6-dichlorophenyl)carbonyl]-O-[2-(5-methyl-5,6,7,8-tetrahydro-1,8-naphthyridin-2-yl)ethyl]-L-tyrosinate as a white foam (250 mg, 55%); ¹H NMR Spectrum (CDCl3) 1.25 (d, 3H), 1.60-1.69 (m, 1H), 1.88-1.97 (m, 1H), 2.80-2.89 (m, 1H), 3.01 (t, 1H), 3.14-3.25 (m, 2H), 3.35-3.48 (m, 2H), 3.74 (s, 3H), 4.33 (t, 2H), 5.14 (dd, 1H), 5.41 (bs, 1H), 6.30 (d, 1H), 6.47 (d, 1H), 6.82 (d, 2H), 7.09 (d, 2H), 7.20 (d, 1H), 7.25 (dd, 1H), 7.28-7.33 (m, 2H).

The starting material, tert-butyl 7-(2-hydroxyethyl)-4-methyl-3,4-dihydro-1,8-naphthyridine-1(2H)-carboxylate was prepared according to the following scheme:

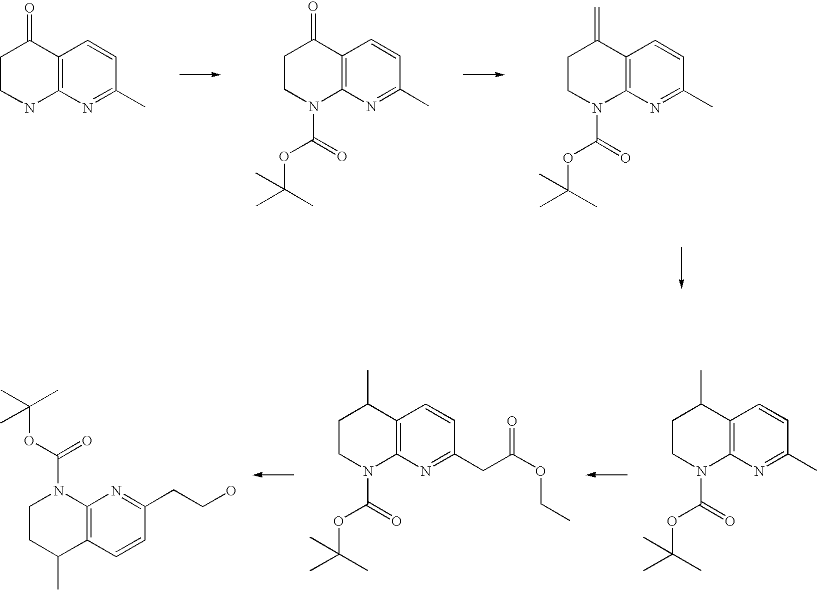

To a solution of 7-methyl-2,3-dihydro-1H-1,8-naphthyridin-4-one (590 mg, 3.64 mmol) (Journal of Heterocyclic Chemistry 1996, 33(4), 1185-1189) in THF (8 ml) were added catalytic DMAP (44 mg, 0.36 mmol) and BOC₂O (1.03 g, 4.73 mmol). The reaction mixture was stirred overnight at 50° C. After removal of solvent, the residue was purified by silica gel flash chromatography (20 to 35% ethyl acetate in petroleum ether) to give tert-butyl 7-methyl-4-oxo-3,4-dihydro-1,8-naphthyridine-1(2H)-carboxylate as a solid (736 mg, 77%); ¹H NMR Spectrum (CDCl3) 1.56 (s, 9H), 2.56 (s, 3H), 2.76 (t, 2H), 4.14 (t, 2H), 6.99 (d, 1H), 8.13 (d, 1H).

To a solution of methyltriphenylphosphonium iodide (2.45 g, 6.05 mmol) in THF (30 ml) was slowly added at −20° C. a solution of BuLi 2.5M in hexane (2.42 ml, 6.05 mmol). After 0.5 hour at −10° C., tert-butyl 7-methyl-4-oxo-3,4-dihydro-1,8-naphthyridine-1(2H)-carboxylate (735 mg, 2.80 mmol) in THF (10 ml) was added dropwise. The reaction mixture was allowed to warm up to room temperature and was left to stir overnight. Dilution in ethyl acetate was followed by a wash with water, then brine. The organic layer was dried, evaporated and purified by silica gel flash chromatography (15 to 30% ethyl acetate in petroleum ether) to give tert-butyl 7-methyl-4-methylidene-3,4-dihydro-1,8-naphthyridine-1(2H)-carboxylate as a yellow solid (196 mg, 28%); ¹H NMR Spectrum (CDCl3) 1.53 (s, 9H), 2.50 (s, 3H), 2.70 (t, 2H), 3.82 (t, 2H), 5.01 (s, 1H), 5.57 (s, 1H), 6.80 (d, 1H), 7.84 (d, 1H).

A mixture of tert-butyl 7-methyl-4-methylidene-3,4-dihydro-1,8-naphthyridine-1(2H)-carboxylate (357 mg, 1.37 mmol) and Pd/C 10% (50 mg) in methanol (20 ml) was hydrogenated under 1 bar of H₂ for 2 hours, filtered through celite and purified by silica gel flash chromatography (15 to 25% ethyl acetate in petroleum ether) to afford tert-butyl 4,7-dimethyl-3,4-dihydro-1,8-naphthyridine-1(2H)-carboxylate as an oil (312 mg, 87%); ¹H NMR Spectrum (CDCl3) 1.28 (d, 3H), 1.52 (s, 9H), 1.53-1.60 (m, 1H), 2.00-2.07 (m, 1H), 2.48 (s, 3H), 2.80-2.88 (m, 1H), 3.71-3.80 (m, 2H), 6.84 (d, 1H), 7.36 (d, 1H).

A solution of LDA was prepared by adding BuLi 2.5M in hexane (1.92 ml, 4.81 mmol) to diisopropylamine (0.67 ml, 4.81 mmol) in THF (7 ml) at −20° C. and stirring for 1 hour at −20° C. The mixture was cooled to −60° C., and slowly added dropwise to a solution of tert-butyl 4,7-dimethyl-3,4-dihydro-1,8-naphthyridine-1(2H)-carboxylate (451 mg, 1.72 mmol) and diethylcarbonate (0.77 ml, 6.36 mmol) in THF (5 ml). After 1.5 hours, the reaction mixture was quenched at −60° C. with aqueous saturated ammonium chloride (5 ml). After addition of ethyl acetate and water, the aqueous layer was extracted twice with ethyl acetate. The organic layer was dried, concentrated and purified by silica gel flash chromatography (10 to 25% ethyl acetate in petroleum ether) to give tert-butyl 7-(2-ethoxy-2-oxoethyl)-4-methyl-3,4-dihydro-1,8-naphthyridine-1(2H)-carboxylate as an oil (459 mg, 80%); ¹H NMR Spectrum (CDCl3) 1.26 (t, 3H), 1.29 (d, 3H), 1.51 (s, 9H), 1.53-1.62 (m, 1H), 2.01-2.09 (m, 1H), 2.82-2.90 (m, 1H), 3.71-3.80 (m, 4H), 4.17 (q, 2H), 7.01 (d, 1H), 7.45 (d, 1H).

A solution of 2N LiBH4 in THF (0.76 ml, 1.51 mmol) was added to a stirred solution of tert-butyl 7-(2-ethoxy-2-oxoethyl)-4-methyl-3,4-dihydro-1,8-naphthyridine-1(2H)-carboxylate (421 mg, 1.26 mmol) in THF (10 ml) under nitrogen. The reaction was stirred at room temperature overnight, then carefully quenched with water, and dried with an excess of MgSO₄ during 15 minutes. The mixture was filtered, concentrated and purified by silica gel flash chromatography (30 to 50% ethyl acetate in petroleum ether) to afford tert-butyl 7-(2-hydroxyethyl)-4-methyl-3,4-dihydro-1,8-naphthyridine-1(2H)-carboxylate as a colourless oil (279 mg, 76%); ¹H NMR Spectrum (CDCl3) 1.29 (d, 3H), 1.54 (s, 9H), 1.56-1.64 (m, 1H), 1.98-2.07 (m, 1H), 2.81-2.89 (m, 1H), 1.92 (t, 2H), 3.73-3.85 (m, 2H), 3.94-4.03 (m, 2H), 5.57 (bs, 1H), 6.80 (d, 1H), 7.41 (d, 1H).

### Example 45

### N-[(2-chlorophenyl)carbonyl]-4-[3-(5,6,7,8-tetrahydro-1,8-naphthyridin-2-yl)propyl]-L-phenylalanine

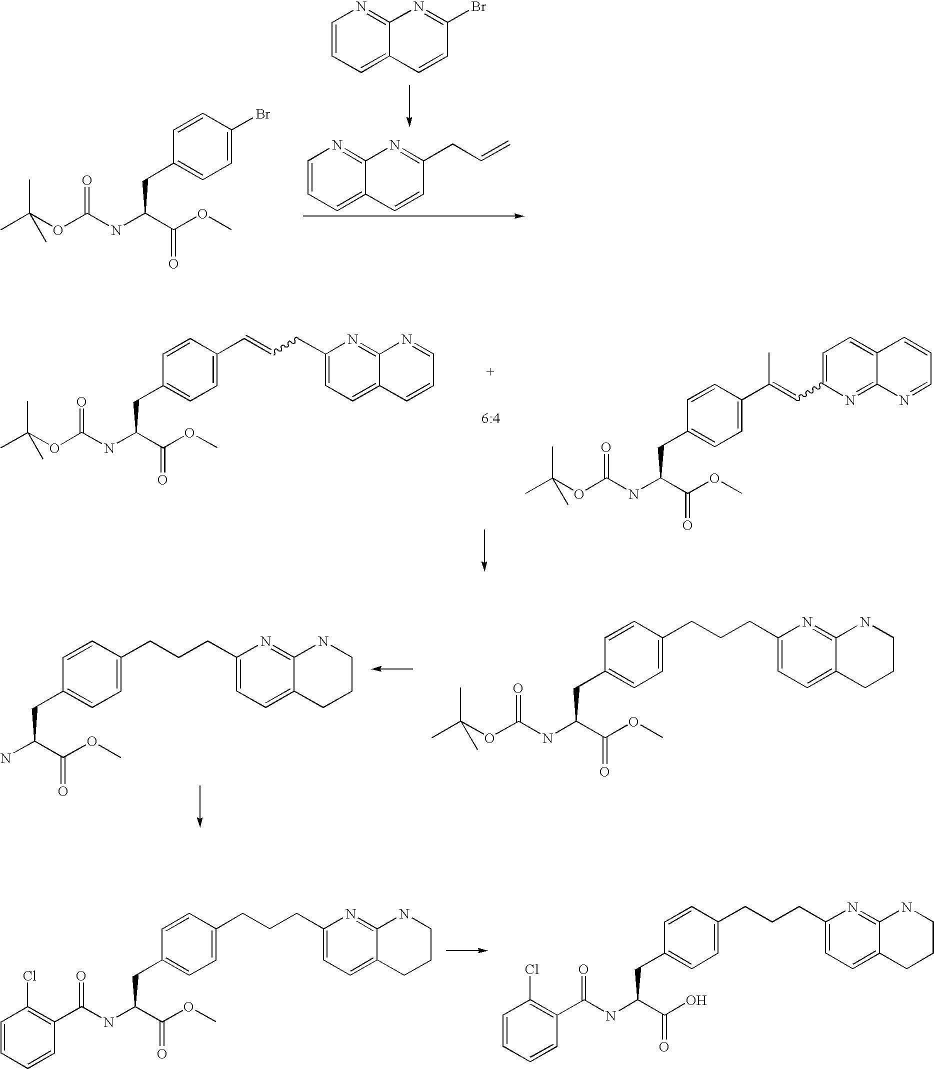

To a solution of methyl N-[(2-chlorophenyl)carbonyl]-4-[3-(5,6,7,8-tetrahydro-1,8-naphthyridin-2-yl)propyl]-L-phenylalaninate (42 mg, 0.09 mmol) in DMF (1 ml) was added LiOH (18 mg). The reaction mixture was stirred at room temperature for 4 hours, then directly purified by C18 reverse phase chromatography (basic conditions) to afford the title compound as a white solid (18 mg, 44%); Mass Spectrum [M+H]⁺=478; ¹H NMR Spectrum (DMSO-d6) 1.70-1.78 (m, 2H), 1.79-1.89 (m, 2H), 2.42 (t, 2H), 2.54 (t, 2H), 2.60 (t, 2H), 2.92 (dd, 1H), 3.14 (dd, 1H), 3.23 (bs, 2H), 4.53 (ddd, 1H), 6.24 (d, 1H), 6.38 (bs, 1H), 7.03 (d, 1H), 7.09 (d, 2H), 7.19 (d, 2H), 7.22 (dd, 1H), 7.34 (ddd, 1H), 7.41 (ddd, 1H), 7.44 (dd, 1H), 8.57 (d, 1H).

The methyl N-[(2-chlorophenyl)carbonyl]-4-[3-(5,6,7,8-tetrahydro-1,8-naphthyridin-2-yl)propyl]-L-phenylalaninate was prepared as follows:

A solution of 2-bromo-1,8-naphthyridine (800 mg, 3.83 mmol), 2-allyl-4,4,5,5-tetramethyl-1,3,2-dioxaborolane (0.96 ml, 5.36 mmol), Pd(Cl)₂dppf (310 mg, 0.38 mmol) and cesium fluoride (1.74 g, 11.50 mmol) in acetonitrile (20 ml) was degassed, then heated to 80° C. for 2 hours. The reaction mixture was cooled down, filtered, concentrated and purified by silica gel flash chromatography (50 to 70% ethyl acetate in petroleum ether) to afford 2-prop-2-en-1-yl-1,8-naphthyridine as a yellow oil (475 mg, 73%); Mass Spectrum [M+H]⁺=171; ¹H NMR Spectrum (CDCl3) 3.86 (ddd, 2H), 5.19 (ddd, 1H), 5.26 (ddd, 1H), 6.14-6.24 (m, 1H), 7.43 (d, 1H), 7.46 (dd, 1H), 8.12 (d, 1H), 8.16 (dd, 1H), 9.09 (dd, 1H).

A solution of 2-prop-2-en-1-yl-1,8-naphthyridine (475 mg, 2.79 mmol), methyl 4-bromo-N-(tert-butoxycarbonyl)-L-phenylalaninate (1000 mg, 2.79 mmol), tri-O-tolylphosphine (170 mg, 0.56 mmol), Palladium(II) acetate (63 mg, 0.28 mmol) and DIPEA (0.49 ml, 2.79 mmol) in acetonitrile (12 ml) was degassed, then sealed and heated to 150° C. for 20 minutes in microwave. After concentration, a purification by C18 reverse phase chromatography (basic conditions) followed by a purification by silica gel (10% MeOH in DCM) afforded methyl N-(tert-butoxycarbonyl)-4-[3-(1,8-naphthyridin-2-yl)prop-1-en-1-yl]-L-phenylalaninate as a impure beige solid (280 mg, 22%); Mass Spectrum [M+H]⁺=448. (NMR analyses after hydrogenation of this complex mixture of alkenes revealed a 6:4 mixture of the expected compound together with a isomer of position; methyl N-(tert-butoxycarbonyl)-4-[1-methyl-2-(1,8-naphthyridin-2-yl)ethenyl]-L-phenylalaninate).

A mixture of methyl N-(tert-butoxycarbonyl)-4-[3-(1,8-naphthyridin-2-yl)prop-1-en-1-yl]-L-phenylalaninate (280 mg, 0.63 mmol) and Pd/C 10% (80 mg) in ethanol (20 ml) was hydrogenated under 4 bar of H₂ for 4 hours, filtered through celite, evaporated and purified by silica gel flash chromatography (2 to 5% methanol in DCM) to afford methyl N-(tert-butoxycarbonyl)-4-[3-(5,6,7,8-tetrahydro-1,8-naphthyridin-2-yl)propyl]-L-phenylalaninate (190 mg, 67%); Mass Spectrum [M+H]⁺=454.

Methyl N-(tert-butoxycarbonyl)-4-[3-(5,6,7,8-tetrahydro-1,8-naphthyridin-2-yl)propyl]-L-phenylalaninate (190 mg, 0.42 mmol) in DCM (1 ml) was stirred with TFA (1 ml) for 4 hours at room temperature. Evaporation was followed by dissolution in DCM and slow addition of a solution of NH₃ 7N in MeOH at 0° C. Removal of salts by filtration was followed by a purification by silica gel flash chromatography (2 to 5% methanol in DCM) to give methyl 4-[3-(5,6,7,8-tetrahydro-1,8-naphthyridin-2-yl)propyl]-L-phenylalaninate (80 mg, 54%); Mass Spectrum [M+H]⁺=354.

To a solution of methyl 4-[3-(5,6,7,8-tetrahydro-1,8-naphthyridin-2-yl)propyl]-L-phenylalaninate (150 mg, 0.31 mmol), 2-chlorobenzoic acid (53 mg, 0.34 mmol) and N-methylmorpholine (13 μl, 0.12 mmol) in DMF (1 ml) was added TBTU (128 mg, 0.40 mmol). The reaction mixture was stirred at room temperature for 16 hours. A purification by C18 reverse phase chromatography (basic conditions) afforded the separation of:

(i) methyl N-[(2-chlorophenyl)carbonyl]-4-[1-methyl-2-(5,6,7,8-tetrahydro-1,8-naphthyridin-2-yl)ethyl]-L-phenylalaninate (37 mg, 25%); ¹H NMR Spectrum (DMSO-d6) 1.10 (d, 3H), 1.69-1.77 (m, 2H), 2.54-2.62 (m, 3H), 2.71 (dd, 1H), 2.93 (dd, 1H), 3.06-3.17 (m, 2H), 3.20-3.26 (m, 2H), 3.65 (s, 3H), 4.63 (ddd, 1H), 6.17 (dd, 1H), 6.27 (bs, 1H), 6.95 (dd, 1H), 7.14-7.27 (m, 5H), 7.23-7.38 (m, 1H), 7.43 (ddd, 1H), 7.46 (dd, 1H), 8.88 (d, 1H); and

(ii) methyl N-[(2-chlorophenyl)carbonyl]-4-[3-(5,6,7,8-tetrahydro-1,8-naphthyridin-2-yl)propyl]-L-phenylalaninate (42 mg, 28%); ¹H NMR Spectrum (DMSO-d6) 1.70-1.78 (m, 2H), 1.80-1.90 (m, 2H), 2.42 (t, 2H), 2.55 (t, 2H), 2.59 (t, 2H), 2.94 (dd, 1H), 3.11 (dd, 1H), 3.22 (bs, 1H), 3.66 (s, 3H), 4.63 (ddd, 1H), 6.21-6.27 (m, 2H), 7.02 (d, 1H), 7.12 (d, 2H), 7.16-7.23 (m, 3H), 7.35 (ddd, 1H), 7.42 (ddd, 1H), 7.46 (dd, 1H), 8.90 (d, 1H).

### Example 46

### N-[(2,6-dichlorophenyl)carbonyl]-O-{2-[6-(dimethylamino)pyridin-2-yl]ethyl}-L-tyrosine

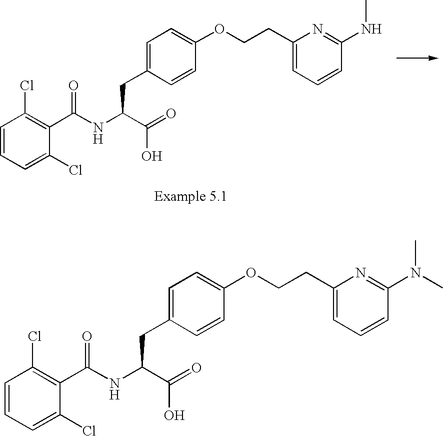

To a solution of N-(2,6-dichlorobenzoyl)-O-{2-[6-(methylamino)pyridin-2-yl]ethyl}-L-tyrosine (Example 5.1) (77 mg, 0.16 mmol) in DCM (1 ml) were successively added at room temperature acetic acid (68 μl), then 37% aqueous formaldehyde (68 μl) and 5 seeds of 4 A molecular sieves. After 0.5 hour, NaBH(OAc)₃ (68 mg, 0.32 mmol) was added. The reaction reached completion after 0.5 hour. A filtration was followed by evaporation, and two consecutive purifications by C18 reverse phase chromatography (acidic conditions then basic conditions) afforded the title compound as a white solid (39 mg, 51%); Mass Spectrum [M+H]⁺=502; ¹H NMR Spectrum (DMSOd6+TFAd) 2.90 (dd, 1H), 2.97 (t, 2H), 3.00 (s, 6H), 3.05 (dd, 1H), 4.27 (t, 2H), 4.38-4.45 (m, 1H), 6.46 (d, 1H), 6.51 (d, 1H), 6.79 (d, 2H), 7.16 (d, 2H), 7.37 (dd, 1H), 7.40-7.45 (m, 2H), 8.45 (bs, 1H).

### Example 47

### O-{2-[6-(cyclopentylamino)pyridin-2-yl]ethyl}-N-[(2,6-dichlorophenyl)carbonyl]-L-tyrosine

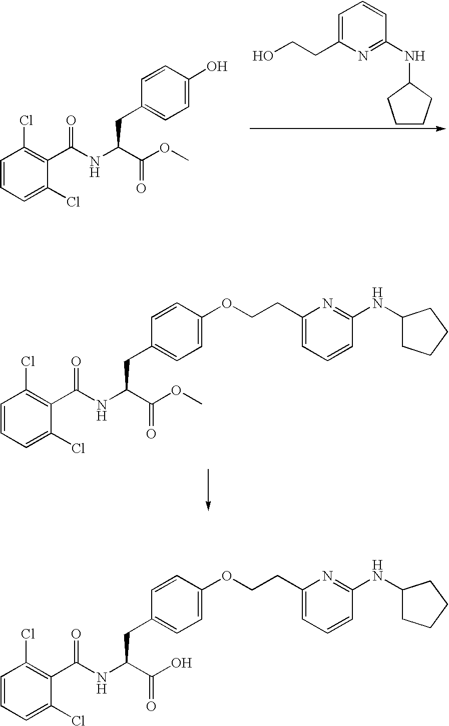

To a solution of methyl O-{2-[6-(cyclopentylamino)pyridin-2-yl]ethyl}-N-[(2,6-dichlorophenyl)carbonyl]-L-tyrosinate (158 mg, 0.28 mmol) in MeOH (2 ml) was added LiOH 2M in water (0.57 ml). The reaction mixture was stirred at room temperature overnight, then filtered, concentrated and the residue purified by C18 reverse phase chromatography (basic conditions) to afford the title compound as a white solid (123 mg, 80%); Mass Spectrum [M+H]⁺=542; ¹H NMR Spectrum (DMSO-d6) 1.36-1.46 (m, 2H), 1.47-1.57 (m, 2H), 1.60-1.70 (m, 2H), 1.85-1.97 (m, 2H), 2.86 (dd, 1H), 2.92 (t 2H), 3.05 (dd, 1H), 3.99-4.09 (m, 1H), 4.25 (t, 2H), 4.55-4.63 (m, 1H), 6.28 (d, 1H), 6.36 (d, 1H), 6.39 (d, 1H), 6.83 (d, 2H), 7.13 (d, 2H), 7.27 (dd, 1H), 7.39 (dd, 1H), 7.41-7.45 (m, 2H), 8.99 (bs, 1H)

The methyl O-{2-[6-(cyclopentylamino)pyridin-2-yl]ethyl}-N-[(2,6-dichlorophenyl)carbonyl]-L-tyrosinate used as starting material was prepared as follows.

To a solution of 2-[6-(cyclopentylamino)pyridin-2-yl]ethanol (169 mg, 0.82 mmol), methyl N-(2,6-dichlorobenzoyl)-L-tyrosinate (151 mg, 0.41 mmol) and triphenylphosphine (239 mg, 0.90 mmol) in DCM (2 ml) was slowly added at 0° C. a solution of DTAD (189 mg, 0.82 mmol) in DCM (1 ml). The reaction mixture was stirred overnight, then solvent was evaporated and the residue was purified by silica gel flash chromatography (0 to 15% ethyl acetate in petroleum ether) to afford methyl O-{2-[6-(cyclopentylamino)pyridin-2-yl]ethyl}-N-[(2,6-dichlorophenyl)carbonyl]-L-tyrosinate as a pale yellow foam (188 mg, 82%); Mass Spectrum [M+H]⁺=556; ¹H NMR Spectrum (DMSO-d6) 1.37-1.46 (m, 2H), 1.47-1.57 (m, 2H), 1.60-1.70 (m, 2H), 1.85-1.96 (m, 2H), 2.88 (dd, 1H), 2.92 (t, 2H), 3.04 (dd, 1H), 3.64 (s, 3H), 3.98-4.09 (m, 1H), 4.26 (t, 2H), 4.66 (ddd, 1H), 6.28 (d, 1H), 6.36 (d, 1H), 6.39 (d, 1H), 6.84 (d, 2H), 7.17 (d, 2H), 7.27 (dd, 1H), 7.40 (dd, 1H), 7.43-7.48 (m, 2H), 9.18 (d, 1H).

The 2-[6-(cyclopentylamino)pyridin-2-yl]ethanol was obtained according to the following scheme:

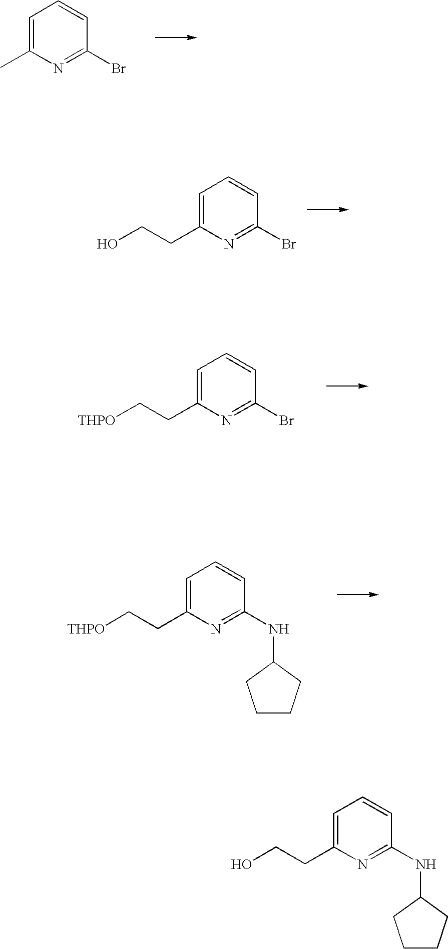

A solution of LDA was prepared by adding BuLi 2.5M in hexane (24.00 ml, 59.9 mmol) to diisopropylamine (8.14 ml, 58.1 mmol) in THF (50 ml) at 0° C. and stirring 1 hour at 0° C. Then the mixture was cooled to −78° C., and 2-bromo-6-methyl-pyridine (5.00 g, 29.1 mmol) in THF (10 ml) was slowly added dropwise over 15 minutes, and the reaction mixture was stirred for 30 minutes. Then DMF (2.34 ml, 30.2 mmol) in THF (5 ml) was added dropwise. After 30 minutes at −78° C., MeOH (30 ml), AcOH (1.78 ml, 31.1 mmol) and NaBH4 (1.04 g, 27.6 mmol) were added successively, and the temperature raised to 10° C. for 1.5 hours. The reaction mixture was quenched by careful addition of saturated aqueous bicarbonate under strong stirring. After addition of ethyl acetate and water, the aqueous layer was extracted twice with ethyl acetate. The organic layer was washed with brine, dried, concentrated and purified by silica gel flash chromatography (10 to 50% diethyl ether in petroleum ether) to give 2-(6-bromopyridin-2-yl)ethanol as a yellow oil (2.67 g, 45%); Mass Spectrum [M+H]⁺=203; ¹H NMR Spectrum (DMSO-d6) 2.84 (t, 2H), 3.64-3.75 (m, 2H), 4.67 (t, 1H), 7.32 (d, 1H), 7.46 (d, 1H), 7.65 (dd, 1H).

To a solution of 2-(6-bromopyridin-2-yl)ethanol (808 mg, 4.00 mmol) and 3,4-dihydro-2H-pyran (0.73 ml, 8.00 mmol) in DCM (10 ml) was added catalytic PTSA (38 mg, 0.2 mmol) and the reaction mixture was stirred at room temperature for 5 hours. The organic layer was washed with water, saturated bicarbonate, brine, then dried over MgSO₄ and concentrated. The residue was purified by silica gel flash chromatography (0 to 15% ethyl acetate in petroleum ether) to give 2-bromo-6-[2-(tetrahydro-2H-pyran-2-yloxy)ethyl]pyridine as a pale yellow oil (980 mg, 86%); ¹H NMR Spectrum (DMSO-d6) 1.33-1.68 (m, 6H), 2.97 (t, 2H), 3.36-3.42 (m, 1H), 3.61 (ddd, 1H), 3.65-3.71 (m, 1H), 3.90-3.97 (m, 1H), 4.56-4.60 (m, 1H), 7.36 (d, 1H), 7.47 (d, 1H), 7.67 (dd, 1H).

A solution of 2-bromo-6-[2-(tetrahydro-2H-pyran-2-yloxy)ethyl]pyridine (286 mg, 1.00 mmol), cyclopentylamine (0.12 ml, 1.20 mmol), sodium tert-butoxide (115 mg, 1.20 mmol), Pd(II) acetate (7 mg, 0.03 mmol) and Josiphos (17 mg, 0.03 mmol) in 1,2-dimethoxyethane (2.5 ml) was degassed, then heated to 80° C. for 1.5 hours. The reaction mixture was cooled down, diluted in DCM, filtered, concentrated and purified by silica gel flash chromatography (5 to 20% ethyl acetate in petroleum ether) to afford N-cyclopentyl-6-[2-(tetrahydro-2H-pyran-2-yloxy)ethyl]pyridin-2-amine as a yellow foam (135 mg, 66%); Mass Spectrum [M+H]⁺=291; ¹H NMR Spectrum (DMSO-d6) 1.32-1.72 (m, 12H), 1.84-1.96 (m, 2H), 2.73 (t, 2H), 3.36-3.42 (m, 1H), 3.62-3.71 (m, 2H), 3.87-3.94 (m, 1H), 3.48-4.07 (m, 1H), 4.54-4.58 (m, 1H), 6.24 (d, 1H), 6.29 (d, 1H), 6.34 (d, 1H), 7.24 (dd, 1H).

A solution of N-cyclopentyl-6-[2-(tetrahydro-2H-pyran-2-yloxy)ethyl]pyridin-2-amine (130 mg, 0.45 mmol) in MeOH (5 ml) was stirred with HCl 2N (0.45 ml, 0.90 mmol) for 18 hours at room temperature. After evaporation to dryness, the residue was diluted in DCM, dried over MgSO₄ filtered and evaporated to give 2-[6-(cyclopentylamino)pyridin-2-yl]ethanol as a pale yellow gum, which was used in net step without further purification (85 mg, 92%); Mass Spectrum [M+H]⁺=207; ¹H NMR Spectrum (DMSO-d6) 1.36-1.46 (m, 2H), 1.47-1.57 (m, 2H), 1.61-1.71 (m, 2H), 1.85-1.95 (m, 2H), 2.64 (t, 2H), 3.64-3.72 (m, 2H), 3.95-4.05 (m, 1H), 4.68 (t, 1H), 6.24 (d, 1H), 6.28-6.35 (m, 2H), 7.23 (dd, 1H).

### Example 48

### O-{2-[6-(cyclopropylamino)pyridin-2-yl]ethyl}-N-[(2,6-dichlorophenyl)carbonyl]-L-tyrosine

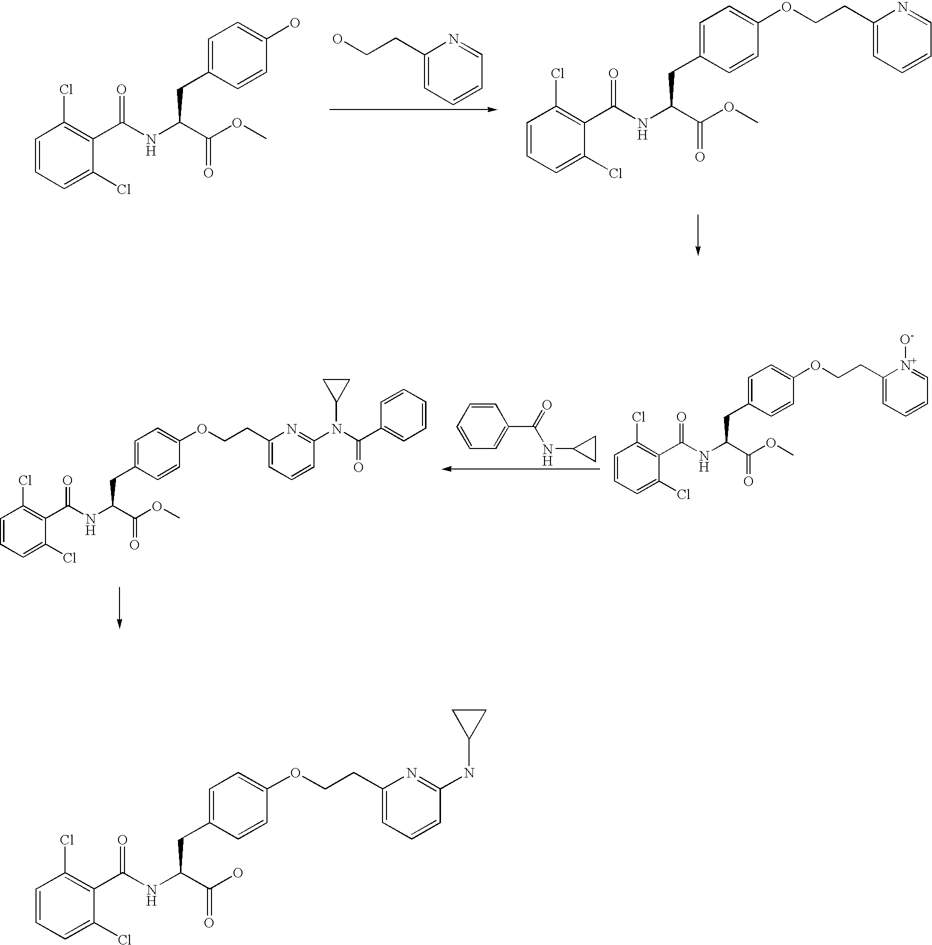

To a solution of methyl -(2-{6-[cyclopropyl(phenylcarbonyl)amino]pyridin-2-yl}ethyl)-N-[(2,6-dichlorophenyl)carbonyl]-L-tyrosinate (50 mg, 0.08 mmol) in THF (4 ml) was added HCl 2N (4 ml). The reaction mixture was heated at 90° C. for 4 hours, then cooled down, concentrated and purified by C18 reverse phase chromatography (acidic conditions then basic conditions) to afford the title compound as a white solid (7 mg, 14%); Mass Spectrum [M+H]⁺=514; ¹H NMR Spectrum (DMSO-d6) 0.37-0.42 (m, 2H), 0.64-0.69 (m, 2H), 2.41-2.48 (m, 1H), 2.89-2.99 (m, 3H), 3.04 (dd, 1H), 4.22 Qt 2H), 4.25-4.35 (m, 1H), 6.46 (d, 1H), 6.51 (d, 1H), 6.67 (s, 1H), 6.77 (d, 2H), 7.15 (d, 2H), 7.35-7.45 (m, 3H).

The methyl O-(2-{6-[cyclopropyl(phenylcarbonyl)amino]pyridin-2-yl}ethyl)-N-[(2,6-dichlorophenyl)carbonyl]-L-tyrosinate was made as follows:

To a solution of pyridine-2-ethanol (3.35 g, 27.2 mmol), methyl N-(2,6-dichlorobenzoyl)-L-tyrosinate (5.0 g, 13.6 mmol) and triphenylphosphine (8.17 g, 31.2 mmol) in DCM (60 ml) was slowly added at 0° C. a solution of DTAD (6.56 g, 28.5 mmol) in DCM (40 ml). The reaction mixture was stirred overnight at room temperature, then solvent was evaporated and the residue was purified by silica gel flash chromatography (0 to 80% ethyl acetate in petroleum ether) to afford methyl N-[(2,6-dichlorophenyl)carbonyl]-O-(2-pyridin-2-ylethyl)-L-tyrosinate as a white foam (4.10 g, 42%); Mass Spectrum [M+H]⁺=473; ¹H NMR Spectrum (DMSO-d6) 2.88 (dd, 1H), 3.04 (dd, 1H), 3.16 (t, 2H), 3.64 (s, 3H), 4.31 (t, 2H), 4.66 (ddd, 1H), 6.84 (d, 2H), 7.17 (d, 2H), 7.24 (ddd, 1H), 7.35 (d, 1H), 7.40 (dd, 1H), 7.43-7.47 (m, 2H), 7.73 (ddd, 1H), 8.51 (ddd, 1H), 9.18 (d, 1H).

To a solution of methyl N-[(2,6-dichlorophenyl)carbonyl]-O-(2-pyridin-2-ylethyl)-L-tyrosinate (600 mg, 1.27 mmol) in acetonitrile (4 ml) was added mCPBA (370 mg, 1.65 mmol). The reaction mixture was heated at 60° C. overnight. After cooling, DCM was added and the organic layer was washed with saturated aqueous sodium thiosulfate, dried over MgSO₄, concentrated and purified by silica gel flash chromatography (0 to 10% methanol in DCM) to afford methyl N-[(2,6-dichlorophenyl)carbonyl]-O-[2-(1-oxidopyridin-2-yl)ethyl]-L-tyrosinate as a white foam (560 mg, 90%); ¹H NMR Spectrum (DMSO-d6) 2.88 (dd, 1H), 3.04 (dd, 1H), 3.23 (t, 2H), 3.64 (s, 3H), 4.29 (t, 2H), 4.66 (dd, 1H), 6.87 (d, 2H), 7.17 (d, 2H), 7.28-7.36 (m, 2H), 7.40 (dd, 1H), 7.43-7.47 (m, 2H), 7.49 (dd, 1H), 8.28 (dd, 1H), 9.18 (d, 1H).

To a solution of N-cyclopropyl benzamide (100 mg, 0.62 mmol) in DCM (2 ml) were added at 0° C. 2,6-lutidine (121 μl, 1.04 mmol) then oxalyl chloride (52 μl, 0.62 mmol). After 30 minutes, methyl N-[(2,6-dichlorophenyl)carbonyl]-O-[2-(1-oxidopyridin-2-yl)ethyl]-L-tyrosinate (181 mg, 0.37 mmol) was added and the reaction mixture was stirred at room temperature for 2 hours, then at 50° C. for 3 hours, showing 50% conversion. The reaction was stopped, cooled down, concentrated and the residue was purified by C18 reverse phase chromatography (basic conditions) to afford methyl O-(2-{6-[cyclopropyl(phenylcarbonyl)amino]pyridin-2-yl}ethyl)-N-[(2,6-dichlorophenyl)carbonyl]-L-tyrosinate (50 mg, 13%); Mass Spectrum [M+H]⁺=632.

### Example 49

### O-[2-(6-aminopyridin-2-yl)ethyl]-N-[(2,6-dichlorophenyl)carbonyl]-L-tyrosine

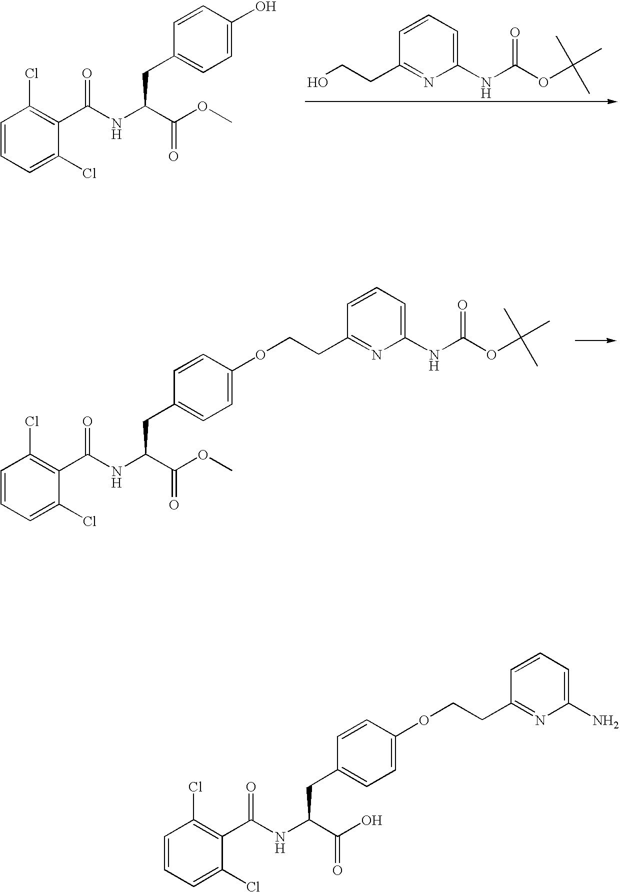

To a solution of methyl O-(2-{6-[(tert-butoxycarbonyl)amino]pyridin-2-yl}ethyl)-N-[(2,6-dichlorophenyl)carbonyl]-L-tyrosinate (50 mg, 0.09 mmol) in DCM (1 ml) was added TFA (1 ml). The reaction mixture was stirred at room temperature for 16 hours. Addition of NH₃ 7M in MeOH until neutral pH was followed by filtration of the salts and evaporation to dryness. The residue was dissolved in DMF/H₂O (1 ml/0.2 ml) and LiOH (18 mg) was added. After 1 hour at room temperature, the reaction mixture was filtered and purified by C18 reverse phase chromatography (basic conditions) to afford the title compound as a white solid (29 mg, 58%); Mass Spectrum [M+H]⁺=474; ¹H NMR Spectrum (DMSO-d6) 2.87-2.95 (m, 3H), 3.04 (dd, 1H), 4.21 (t, 2H), 4.41 (bs, 1H), 5.83 (bs, 2H), 6.28 (d, 1H), 6.43 (d, 1H), 6.78 (d, 2H), 7.16 (d, 2H), 7.28 (dd, 1H), 7.38 (dd, 1H), 7.41-7.46 (m, 2H), 8.50 (bs, 1H).

The methyl O-(2-{6-[(tert-butoxycarbonyl)amino]pyridin-2-yl}ethyl)-N-[(2,6-dichlorophenyl)carbonyl]-L-tyrosinate was made as follows:

To a solution of tert-butyl [6-(2-hydroxyethyl)pyridin-2-yl]carbamate-(1.42 g, 5.97 mmol) (described in WO2002/018377 page 47), methyl N-(2,6-dichlorobenzoyl)-L-tyrosinate (2.00 g, 5.43 mmol) and triphenylphosphine (2.99 g, 11.4 mmol) in DCM (25 ml) was slowly added at 0° C. a solution of ADDP (2.88 g, 11.4 mmol) in DCM (10 ml). The reaction mixture was stirred overnight at room temperature, then solvent was evaporated and the residue was purified by silica gel flash chromatography (0 to 50% ethyl acetate in petroleum ether) to afford methyl O-(2-{6-[(tert-butoxycarbonyl)amino]pyridin-2-yl}ethyl)-N-[(2,6-dichlorophenyl)carbonyl]-L-tyrosinate as a pale yellow solid (2.53 g, 79%); Mass Spectrum [M+H]⁺=588; ¹H NMR Spectrum (DMSO-d6) 1.45 (s, 9H), 2.88 (dd, 1H), 3.01-3.09 (m, 3H), 3.67 (s, 3H), 4.28 (t, 2H), 4.66 (ddd, 1H), 6.84 (d, 2H), 6.98 (dd, 1H), 7.17 (d, 2H), 7.40 (dd, 1H), 7.43-7.48 (m, 2H), 7.61-7.69 (m, 2H), 9.17 (d, 1H), 9.62 (s, 1H).

### Example 50

### O-(2-{6-[(cyclopropylmethyl)amino]pyridin-2-yl}ethyl)-N-[(2,6-dichlorophenyl)carbonyl]-L-tyrosine

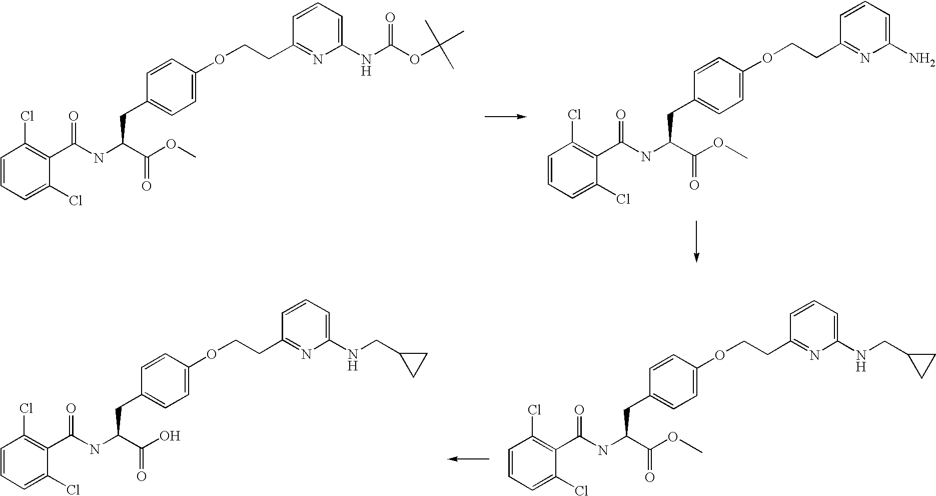

To a solution of methyl O-(2-{6-[(cyclopropylmethyl)amino]pyridin-2-yl}ethyl)-N-[(2,6-dichlorophenyl)carbonyl]-L-tyrosinate (150 mg, 0.28 mmol) in DMA/H₂O (1.0 ml/0.3 ml) was added LiOH (30 mg). The reaction mixture was stirred at 50° C. for 1.5 hours, then cooled down and directly purified by C18 reverse phase chromatography (basic conditions) to afford the title compound as a white solid (97 mg, 65%); Mass Spectrum [M+H]⁺=528; ¹ H NMR Spectrum (DMSO-d6) 0.16-0.22 (m, 2H), 0.36-0.44 (m, 2H), 0.98-1.07 (m, 1H), 2.86-2.94 (m, 3H), 3.04 (dd, 1H), 3.09 (t 2H), 4.24 (t 2H), 4.46 (bs, 1H), 6.31 (d, 1H), 6.40 (d, 1H), 6.47 (t 1H), 6.80 (d, 2H), 7.17 (d, 2H), 7.27 (dd, 1H), 7.38 (dd, 1H), 7.41-7.45 (m, 2H), 8.64 (bs, 1H).

The methyl O-(2-{6-[(cyclopropylmethyl)amino]pyridin-2-yl}ethyl)-N-[(2,6-dichlorophenyl)carbonyl]-L-tyrosinate used as starting material was prepared as follows:

Methyl O-(2-{6-[(tert-butoxycarbonyl)amino]pyridin-2-yl}ethyl)-N-[(2,6-dichlorophenyl)carbonyl]-L-tyrosinate (1000 mg, 1.70 mmol) (described in example 49) in DCM (5 ml) was stirred with a solution of HCl 4M in dioxane (5 ml) at room temperature overnight. Then more DCM was added and a solution of NH₃ 7N in MeOH was added dropwise to adjust to pH 9. Evaporation was followed by a purification by silica gel flash chromatography (0 to 100% ethyl acetate in petroleum ether) to give methyl O-[2-(6-aminopyridin-2-yl)ethyl]-N-[(2,6-dichlorophenyl)carbonyl]-L-tyrosinate as a gum (512 mg, 62%); Mass Spectrum [M+H]⁺=488; ¹H NMR Spectrum (DMSO-d6) 2.83-2.94 (m, 3H), 3.04 (dd, 1H), 3.64 (s, 3H), 4.22 (t, 2H), 4.66 (ddd, 1H), 5.83 (bs, 2H), 6.28 (d, 1H), 6.43 (d, 1H), 6.83 (d, 2H), 7.17 (d, 2H), 7.27 (dd, 1H), 7.40 (dd, 1H), 7.43-7.49 (m, 2H), 9.18 (d, 1H).

To a solution of methyl O-[2-(6-aminopyridin-2-yl)ethyl]-N-[(2,6-dichlorophenyl)carbonyl]-L-tyrosinate (200 mg, 0.42 mmol) in DCM (2 ml) were successively added at room temperature acetic acid (136 μl), then cyclopropane carboxaldehyde (34 μl, 0.46 mmol) and 5 seeds of 4 A molecular sieves. After 0.5 hour, NaBH(OAc)₃ (98 mg, 0.46 mmol) was added. The reaction reached completion after 0.5 hour. A few drops of water were added, followed by a filtration, evaporation and a purification by C18 reverse phase chromatography (acidic conditions) to afford methyl O-(2-{6-[(cyclopropylmethyl)amino]pyridin-2-yl}ethyl)-N-[(2,6-dichlorophenyl)carbonyl]-L-tyrosinate as a colourless gum (130 mg, 57%); Mass Spectrum [M+H]⁺=542; ¹H NMR Spectrum (DMSO-d6) 0.16-0.22 (m, 2H), 0.36-0.44 (m, 2H), 0.98-1.08 (m, 1H), 2.88 (dd, 1H), 2.92 (t, 2H), 3.04 (dd, 1H), 3.09 (t, 2H), 3.64 (s, 3H), 4.25 (t, 2H), 4.66 (dd, 1H), 6.32 (d, 1H), 6.40 (d, 1H), 6.47 (t, 1H), 6.84 (d, 2H), 7.17 (d, 2H), 7.27 (dd, 1H), 7.40 (dd, 1H), 7.43-7.48 (m, 2H), 9.18 (d, 1H).

### Example 51

### N-[(2,6-dichlorophenyl)carbonyl]-O-(2-{6-[(2-methoxyethyl)amino]pyridin-2-yl}ethyl)-L-tyrosine

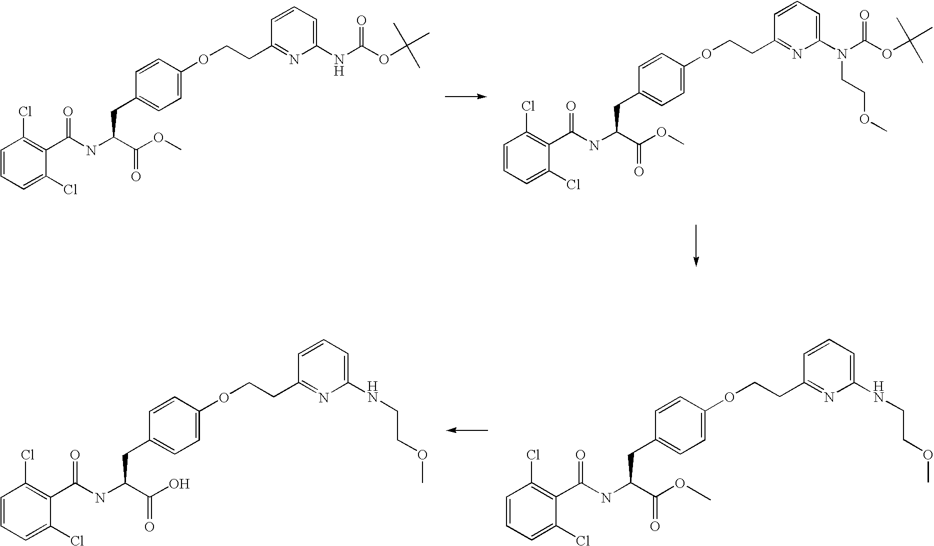

To a solution of methyl N-[(2,6-dichlorophenyl)carbonyl]-O-(2-{6-[(2-methoxyethyl)amino]pyridin-2-yl}ethyl)-L-tyrosinate (55 mg, 0.10 mmol) in DMA/H₂O (0.5 ml/0.1 ml) was added LiOH (30 mg). The reaction mixture was stirred at 50° C. for 1 hour, then cooled down and directly purified by C18 reverse phase chromatography (basic conditions) to afford the title compound as a white solid (30 mg, 56%); Mass Spectrum [M+H]⁺=532; ¹H NMR Spectrum (DMSO-d6) 2.86 (dd, 1H), 2.93 (t, 2H), 3.05 (dd, 1H), 3.24 (s, 3H), 3.38-3.47 (m, 2H), 3.42-3.47 (m, 2H), 4.25 (t, 2H), 4.57 (ddd, 1H), 6.33 (d, 1H), 6.40-6.45 (m, 2H), 6.83 (d, 2H), 7.18 (d, 2H), 7.28 (dd, 1H), 7.38 (dd, 1H), 7.41-7.46 (m, 2H), 8.92 (bs, 1H)

The methyl N-[(2,6-dichlorophenyl)carbonyl]-O-(2-{6-[(2-methoxyethyl)amino]pyridin-2-yl}ethyl)-L-tyrosinate was prepared as follows:

To a solution of methyl O-(2-{6-[(tert-butoxycarbonyl)amino]pyridin-2-yl}ethyl)-N-[(2,6-dichlorophenyl)carbonyl]-L-tyrosinate (300 mg, 0.51 mmol) (described in example 49) in DMF (3 ml) was added at 0° C. NaH (60% in oil, 23 mg, 0.56 mmol). After 0.5 hour, bromoethyl methylether (59 μl, 0.62 mmol) was added and the reaction mixture was stirred at room temperature for 2 hours. A few drops of water were added and the solution was directly purified by C18 reverse phase chromatography (acidic conditions) to afford methyl O-(2-{6-[(tert-butoxycarbonyl)(2-methoxyethyl)amino]pyridin-2-yl}ethyl)-N-[(2,6-dichlorophenyl)carbonyl]-L-tyrosinate (200 mg, 61%); Mass Spectrum [M+H]⁺=646.

To a solution of methyl O-(2-{6-[(tert-butoxycarbonyl)(2-methoxyethyl)amino]pyridin-2-yl}ethyl)-N-[(2,6-dichlorophenyl)carbonyl]-L-tyrosinate (200 mg, 0.31 mmol) in DCM (1 ml) was added TFA (0.5 ml). The reaction mixture was stirred at room temperature for 2 hours. Addition of NH₃ 7M in MeOH until neutral pH was followed by filtration of the salts and evaporation to dryness. A purification by C18 reverse phase chromatography (acidic conditions) afforded methyl N-[(2,6-dichlorophenyl)carbonyl]-O-(2-{6-[(2-methoxyethyl)amino]pyridin-2-yl}ethyl)-L-tyrosinate (58 mg, 34%); Mass Spectrum [M+H]⁺=546.

### Example 52

### N-[(2,6-dichlorophenyl)carbonyl]-O-[2-(6-{[2-(dimethylamino)ethyl]amino}pyridin-2-yl)ethyl]-L-tyrosine

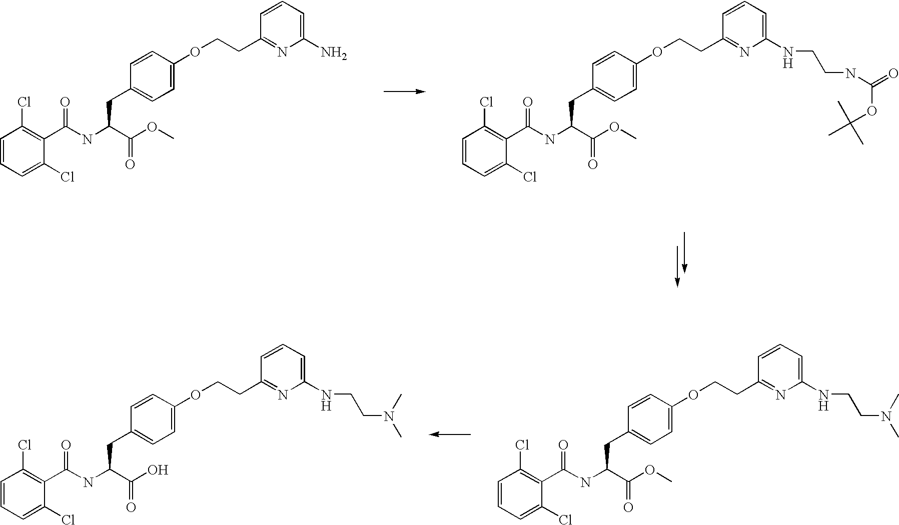

To a solution of methyl N-[(2,6-dichlorophenyl)carbonyl]-O-[2-(6-{[2-(dimethylamino)ethyl]amino}pyridin-2-yl)ethyl]-L-tyrosinate (50 mg, 0.09 mmol) in DMA/H₂O (0.5 ml/0.2 ml) was added LiOH (20 mg). The reaction mixture was stirred at room temperature for 4 hours, then directly purified by C18 reverse phase chromatography (basic conditions) to afford the title compound as a white solid (3 mg, 6%); Mass Spectrum [M+H]⁺=545; ¹H NMR Spectrum (DMSO-d6) 2.26 (s, 6H), 2.52-2.55 (m partially hidden by DMSOd5, 2H), 2.90 (dd, 1H), 2.93 (t, 2H), 3.02 (dd, 1H), 3.29-3.39 (m, 2H), 4.28 (t, 2H), 4.47-4.54 (m, 1H), 6.28-6.33 (m, 2H), 6.42 (d, 1H), 6.79 (d, 2H), 7.16 (d, 2H), 7.28 (dd, 1H), 7.38 (dd, 1H), 7.41-7.47 (m, 2H), 8.65 (d, 1H).

The methyl N-[(2,6-dichlorophenyl)carbonyl]-O-[2-(6-{[2-(dimethylamino)ethyl]amino}pyridin-2-yl)ethyl]-L-tyrosinate was prepared as follows:

To a solution of methyl O-[2-(6-aminopyridin-2-yl)ethyl]-N-[(2,6-dichlorophenyl)carbonyl]-L-tyrosinate (300 mg, 0.61 mmol) (described in example 49) in DCM (4 ml) were successively added at room temperature acetic acid (300 μl), then tert-butyl N-(2-oxoethyl)carbamate (196 mg, 1.23 mmol) and 15 seeds of 4 A molecular sieves. After 0.5 hour, NaBH(OAc)₃ (235 mg, 1.11 mmol) was added. The reaction reached completion after 1 hour. A filtration was followed by evaporation, and a purification by C18 reverse phase chromatography (acidic conditions) afforded methyl O-{2-[6-({2-[(tert-butoxycarbonyl)amino]ethyl}amino)pyridin-2-yl]ethyl}-N-[(2,6-dichlorophenyl)carbonyl]-L-tyrosinate as a white solid (120 mg, 31%); Mass Spectrum [M+H]⁺=631.

Methyl O-{2-[6-({2-[(tert-butoxycarbonyl)amino]ethyl}amino)pyridin-2-yl]ethyl}-N-[(2,6-dichlorophenyl)carbonyl]-L-tyrosinate (60 mg, 0.10 mmol) in DCM (1 ml) was stirred with HCl 4N in dioxane (1 ml) at room temperature overnight. Then solvents were evaporated and the residue was dissolved in acetonitrile (1 ml). 37% aqueous formaldehyde (20 μl), NaBH(OAc)₃ (53 mg, 0.25 mmol) and acetic acid (60 μl) were successively added over 0.5 hour and the reaction mixture was stirred at room temperature overnight. A filtration was followed by a purification by C18 reverse phase chromatography (acidic conditions) to afford methyl N-[(2,6-dichlorophenyl)carbonyl]-O-[2-(6-{[2-(dimethylamino)ethyl]amino}pyridin-2-yl)ethyl]-L-tyrosinate as a colourless gum (50 mg, 70%); Mass Spectrum [M+H]⁺=559; ¹H NMR Spectrum (DMSO-d6) 2.16 (s, 6H), 2.39 (t, 2H), 2.89 (dd, 1H), 2.92 (t, 2H), 3.04 (dd, 1H), 3.29-3.32 (m partially hidden by DMSOd5, 2H), 3.64 (s, 3H), 4.26 (t, 2H), 4.66 (ddd, 1H), 6.23 (t, 1H), 6.32 (d, 1H), 6.41 (d, 1H), 6.84 (d, 2H), 7.17 (d, 2H), 7.28 (dd, 1H), 7.40 (dd, 1H), 7.43-7.47 (m, 2H), 9.17 (d, 1H).

### Example 53

Esters were prepared using different conditions as summarised on the following scheme:

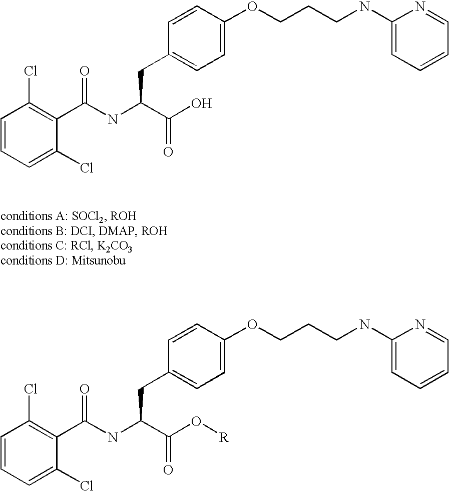

### Example 53.1

### Conditions A

### ethyl N-[(2,6-dichlorophenyl)carbonyl]-O-[3-(pyridin-2-ylamino)propyl]-L-tyrosinate

To a solution of N-(2,6-dichlorobenzoyl)-O-[3-(pyridin-2-ylamino)propyl]-L-tyrosine (example 1.2) (120 mg, 0.25 mmol) in ethanol (0.3 ml) was added SOCl₂ (92 μl, 1.26 mmol) and the mixture was stirred at room temperature for 3 hours. Then DCM was added and the organic layer was washed with saturated aqueous sodium bicarbonate, dried and concentrated. The residue was purified by silica gel flash chromatography (0 to 4% methanol in DCM) to give, after lyophilisation, the title compound as a white powder (123 mg, 97%).

Conditions A were also applied to the synthesis of example 53.2, using isopropanol as the solvent.

### Example 53.3

### Conditions B

### 2-morpholin-4-ylethyl N-[(2,6-dichlorophenyl)carbonyl]-O-[3-(Pyridin-2-ylamino)propyl]-L-tyrosinate

To a solution of N-(2,6-dichlorobenzoyl)-O-[3-(pyridin-2-ylamino)propyl]-L-tyrosine (example 1.2) (100 mg, 0.20 mmol) in THF (2 ml) were successively added DMAP (12 mg, 0.10 mmol), DCC (64 mg, 0.31 mmol) and 2-morpholin-4-yl ethanol (50 μl, 0.41 mmol). The mixture was stirred under nitrogen at room temperature for 6 hours, then a filtration was followed by evaporation of solvents and a purification by C18 reverse phase chromatography (acidic conditions) to afford after lyophilisation the title compound as a white powder (57 mg, 46%).

Conditions B were also applied to the synthesis of example 53.4 to 53.8, using the appropriate alcohol.

### Example 53.9

### Conditions C

### 2-(dimethylamino)-2-oxoethyl N-[(2,6-dichlorophenyl)carbonyl]-O-[3-(pyridin-2-ylamino)propyl]-L-tyrosinate

To a solution of N-(2,6-dichlorobenzoyl)-O-[3-(pyridin-2-ylamino)propyl]-L-tyrosine (example 1.2) (120 mg, 0.25 mmol) in DMF (2 ml) were successively added potassium carbonate (102 mg, 0.74 mmol) and 2-chloro-N,N-dimethylacetamide (28 μl, 0.27 mmol). The mixture was stirred under nitrogen at room temperature for 4 hours, then a filtration was directly followed by a purification by C18 reverse phase chromatography (acidic conditions) to afford after lyophilisation the title compound as a white powder (70 mg, 50%).

### Example 53.10

### Conditions D

### 2-(diethylamino)ethyl N-[(2,6-dichlorophenyl)carbonyl]-O-[3-(pyridin-2-ylamino)propyl]-L-tyrosinate

To a suspension of N-(2,6-dichlorobenzoyl)-O-[3-(pyridin-2-ylamino)propyl]-L-tyrosine (example 1.2) 122 mg, 0.25 mmol) in DMC (1.3 ml) were added under argon atmosphere 2-diethylamino-ethanol (66 μl, 0.5 mmol), PPh₃ (146 mg, 0.55 mmol) and DTAD (115 mg, 0.5 mmol) After evaporation to dryness, the residue was purified by C18 reverse phase chromatography (basic conditions) to afford the title compound as a gum (49 mg, 33%).

Using Conditions A, B, C or D the compounds shown it Table 13 were prepared by reacting the appropriate carboxylic acid with the appropriate alcohol or halide. In Table 13 “OR⁴” refers to the group attached to the carbonyl carbon of the ester.

TABLE 13

| | | | Conditions, | Mass | ¹H NMR Data |
|---|---|---|---|---|---|
| Example | OR⁴ | Name | Yield | Ion | (500 MHz) |
| 53.1 | 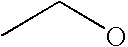 | ethyl N-[(2,6- dichlorophenyl) carbonyl]-O- [3-(pyridin-2- ylamino)propyl]- L-tyrosinate | A, 97% | 516 [M + H]⁺ | 1.16 (t, 3H), 1.91-1.99 (m, 2H), 2.89 (dd, 1H), 3.03 (dd, 1H), 3.33-3.39 (m, 2H), 4.01 (t, 2H), 4.06-4.14 (m, 2H), 4.64 (ddd, 1H), 6.42-6.47 (m, 2H), 6.54 (t, 1H), 6.84 (d, |
| # 2H), 7.18 (d, 2H), 7.34 (ddd, 1H), 7.40 (dd, 1H), 7.43-7.47 (m, 2H), 7.95 (dd, 1H), 9.17 (d, 1H) | | | | | |
| 53.2 | 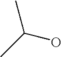 | 1-methylethyl N-[(2,6- dichlorophenyl) carbonyl]-O- [3-(pyridin-2- ylamino)propyl]- L-tyrosinate | A, 70% | 531 [M + H]⁺ | 1.13 (d, 3H), 1.19 (d, 3H), 1.91-1.99 (m, 2H), 2.88 (dd, 1H), 3.01 (dd, 1H), 3.33-3.39 (m partially hidden by H2O, 2H), 4.01 (t, 2H), 4.61 (ddd, 1H), 4.86- |
| #4.94 (m, 1H), 6.42-6.48 (m, 2H), 6.54 (t, 1H), 6.84 (d, 2H), 7.18 (d, 2H), 7.34 (ddd, 1H), 7.40 (dd, 1H), 7.43- 7.48 (m, 2H), 7.95 (dd, 1H), 9.15 (d, 1H) | | | | | |
| 53.3 | 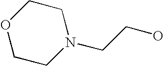 | 2-morpholin-4- ylethyl N- [(2,6- dichlorophenyl) carbonyl]-O- [3-(pyridin-2- ylamino)propyl]- L-tyrosinate | B, 46% | 599 [M − H]⁻ | 1.91-1.99 (m, 2H), 2.34- 2.41 (m, 4H), 2.48 (t partially hidden by DMSOd5, 2H), 2.90 (dd, 1H), 3.04 (dd, 1H), 3.36 (t partially hidden by |
| #H2O, 2H), 3.54 (t, 4H), 4.01 (t, 2H), 4.11-4.21 (m, 2H), 4.66 (ddd, 1H), 6.42-6.47 (m, 2H), 6.52 (t, 2H), 6.85 (d, 2H), 7.18 (d, 2H), 7.34 (ddd, 1H), 7.41 (dd, 1H), 7.43-7.48 (m, 2H), 7.95 (dd, 1H), 9.19 (d, 1H) | | | | | |
| 53.4 | 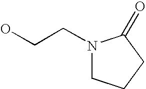 | 2-(2- oxopyrrolidin- 1-yl)ethyl N- [(2,6- dichlorophenyl) carbonyl]-O- [3-(pyridin-2- ylamino)propyl]- L-tyrosinate | B, 24% | 599 [M + H]⁺ | 1.84-1.92 (m, 2H), 1.92- 2.00 (m, 2H), 2.12-2.25 (m, 2H), 2.88 (dd, 1H), 3.04 (dd, 1H), 3.31-3.39 (m, 4H), 3.41 (t, 2H), |
| #4.02 (t, 2H), 4.13-4.21 (m, 2H), 4.65 (ddd, 1H), 6.42-6.48 (m, 2H), 6.54 (t, 1H), 6.85 (d, 2H), 7.18 (d, 2H), 7.34 (ddd, 1H), 7.41 (dd, 1H), 7.43-7.48 (m, 2H), 7.96 (d, 1H), 9.19 (d, 1H) | | | | | |
| 53.5 | 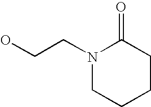 | 2-(2- oxopiperidin- 1-yl)ethyl N- [(2,6- dichlorophenyl) carbonyl]-O- [3-(pyridin-2- ylamino)propyl]- L-tyrosinate | B, 48% | 613 [M + H]⁺ | 1.62-1.73 (m, 4H), 1.90- 2.02 (m, 2H), 2.13-2.24 (m, 2H), 2.88 (dd, 1H), 3.05 (dd, 1H), 3.29 (bs, 2H), 3.37 (t, 2H), 3.44- |
| #3.50 (m, 2H), 3.97-4.05 (m, 2H), 4.12-4.24 (m, 2H), 4.66 (dd, 1H), 6.40- 6.48 (m, 2H), 6.54 (bs, 1H), 6.85 (d, 2H), 7.18 (d, 2H), 7.34 (dd, 1H), 7.37-7.49 (m, 3H), 7.95 (d, 1H), 9.18 (d, 1H) | | | | | |
| 53.6 | 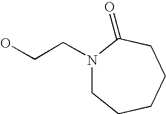 | 2-(2- oxoazepan- 1-yl)ethyl N- [(2,6- dichlorophenyl) carbonyl]-O- [3-(pyridin-2- ylamino)propyl]- L-tyrosinate | B, 40% | 627 [M + H]⁺ | 1.47-1.59 (m, 4H), 1.59- 1.67 (m, 2H), 1.91-2.01 (m, 2H), 2.38-2.44 (m, 2H), 2.89 (dd, 1H), 3.06 (dd, 1H), 3.33-3.40 (m partially |
| # hidden by H2O, 4H), 3.50 (t, 2H), 4.02 (t, 2H), 4.13 (t, 2H), 4.65 (ddd, 1H), 6.42-6.49 (m, 2H), 6.54 (t, 1H), 6.86 (d, 2H), 7.19 (d, 2H), 7.35 (ddd, 1H), 7.41 (dd, 1H), 7.44-7.49 (m, 2H), 7.96 (dd, 1H), 9.20 (d, 1H) | | | | | |
| 53.7 | 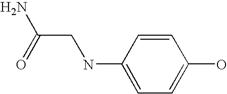 | 4- (glycylamino) phenyl N-[(2,6- dichlorophenyl) carbonyl]-O- [3-(pyridin-2- ylamino)propyl]- L-tyrosinate | B, 44%* | 636 [M + H]⁺ | 1.92-2.00 (m, 2H), 3.07 (dd, 1H), 3.20 (dd, 1H), 3.34-3.40 (m partially hidden by H2O, 2H), 3.75 (s, 2H), 4.03 (t, 2H), 4.81-4.87 (m, 1H), 6.42- |
| # 6.48 (m, 2H), 6.55 (t, 1H), 6.89 (d, 2H), 7.04 (d, 2H), 7.28 (d, 2H), 7.34 (ddd, 1H), 7.43 (dd, 1H), 7.46-7.50 (m, 2H), 7.62 (d, 1H), 7.95 (dd, 1H), 8.05 (bs, 2H), 9.41 (d, 1H) | | | | | |
| 53.8 | 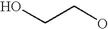 | 2-hydroxyethyl N-[(2,6- dichlorophenyl) carbonyl]-O- [3-(pyridin-2- ylamino)propyl]- L-tyrosinate | B, 45%** | 533 [M + H]⁺ | 1.91-2.00 (m, 2H), 2.90 (dd, 1H), 3.08 (dd, 1H), 3.34-3.39 (m partially hidden by H2O, 2H), 3.53-3.62 (m, 2H), 4.01 (t, 2H), 4.08 (t, 2H), 4.70 (ddd, |
| #1H), 4.81 (t, 1H), 6.42-6.47 (m, 2H), 6.54 (t, 1H), 6.84 (d, 2H), 7.19 (d, 2H), 7.34 (ddd, 1H), 7.40 (dd, 1H), 7.43-7.48 (m, 2H), 7.95 (dd, 1H), 9.15 (d, 1H) | | | | | |
| 53.9 | 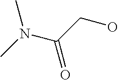 | 2- (dimethylamino)- 2-oxoethyl N-[(2,6- dichlorophenyl) carbonyl]-O- [3-(pyridin-2- ylamino)propyl]- L-tyrosinate | C, 50% | 573 [M + H]⁺ | 1.92-1.99 (m, 2H), 2.84 (s, 3H), 2.90 (dd, 1H), 2.94 (s, 3H), 3.24 (dd, 1H), 3.34-3.40 (m partially hidden by H2O, 2H), 4.01 |
| #(t, 2H), 4.73 (ddd, 1H), 4.80 (d, 1H), 4.93 (d, 1H), 6.43-6.47 (m, 2H), 6.54 (t, 1H), 6.85 (d, 2H), 7.22 (d, 2H), 7.34 (ddd, 1H), 7.39 (dd, 1H), 7.42-7.46 (m, 2H), 7.95 (dd, 1H), 9.20 (d, 1H) | | | | | |
| 53.10 | 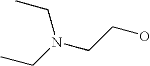 | 2- (diethylamino) ethyl N-[(2,6- dichlorophenyl) carbonyl]-O- [3-(pyridin-2- ylamino)propyl]- L-tyrosinate | D, 61% | 588 [M + H]⁺ | 0.93 (t, 6H), 1.91-1.99 (m, 2H), 2.47 (q, 4H), 2.55 (t, 2H), 2.89 (dd, 1H), 3.04 (dd, 1H), 3.34- 3.39 (m, partially hidden by H2O, 2H), 4.01 (t, |
| # 2H), 4.03-4.13 (m, 2H), 4.64 (ddd, 1H), 6.42-6.47 (m, 2H), 6.54 (t, 1H), 6.84 (d, 2H), 7.18 (d, 2H), 7.34 (ddd, 1H), 7.41 (dd, 1H), 7.43-7.49 (m, 2H), 7.95 (dd, 1H), 9.17 (d, 1H) | | | | | |

*the reaction was performed on the BOC protected chain, which was subsequently removed using TFA.
**the reaction was performed on the THP protected chain, which was subsequently removed using HCl 2N.

The tert-butyl N-[2-[(4-hydroxyphenyl)amino]acetyl]carbamate used in example 53.7 was prepared according to the following scheme:

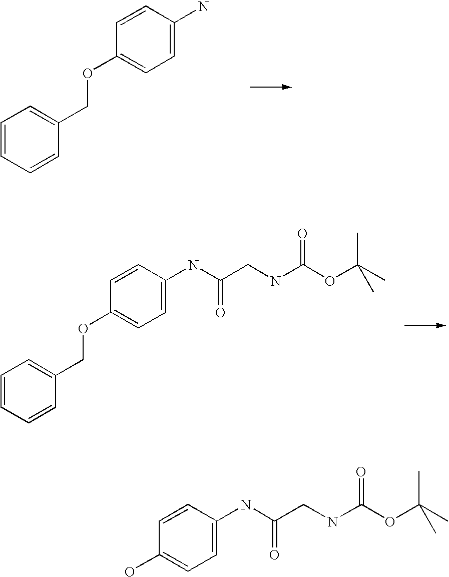

To a suspension of N-(tert-butoxycarbonyl) glycine (1.00 g, 5.71 mmol) in THF (20 ml) were successively added DMAP (348 mg, 2.85 mmol), DCC (1.77 g, 8.56 mmol) and 4-benzyloxyaniline hydrochloride (2.02 g, 8.56 mmol). The mixture was stirred under nitrogen at room temperature for 16 hours. Removal of solvent was followed by a purification by silica gel flash chromatography (20% ethyl acetate in petroleum ether) to give tert-butyl (2-{[4-(benzyloxy)phenyl]amino}-2-oxoethyl)carbamate (1.20 g, 59%); Mass Spectrum [M+H]⁺=357; ¹H NMR Spectrum (DMSO-d6) 1.40 (s, 9H), 3.69 (d, 2H), 5.07 (s, 2H), 6.97 (d, 2H), 7.02 (t, 1H), 7.33 (t, 1H), 7.40 (t, 2H), 7.45 (d, 2H), 7.49 (d, 2H), 8.78 (s, 1H)

A mixture of tert-butyl (2-{[4-(benzyloxy)phenyl]amino}-2-oxoethyl)carbamate (500 mg, 1.4 mmol), Pd/C 10% (50 mg) and ammonium formate (1.33 g, 21.0 mmol) in ethyl acetate (20 ml) was stirred under Argon at room temperature for 16 hours. The reaction mixture was filtered through celite, evaporated to dryness, triturated in a mixture of pentane and diethyl ether and the white precipitate was filtered and dried to afford tert-butyl {2-[(4-hydroxyphenyl)amino]-2-oxoethyl}carbamate (334 mg, 89%); Mass Spectrum [M+H]⁺=267; ¹H NMR Spectrum (DMSO-d6) 1.39 (s, 9H), 3.66 (d, 2H), 6.68 (d, 2H), 7.98 (t, 1H), 7.34 (d, 2H), 9.18 (s, 1H), 9.62 (s, 1H).

### Example 54

### N-[(2,6-dichlorophenyl)carbonyl]-3-(6-{2-[6-(methylamino)pyridin-2-yl]ethoxy}pyridin-3-yl)-L-alanine

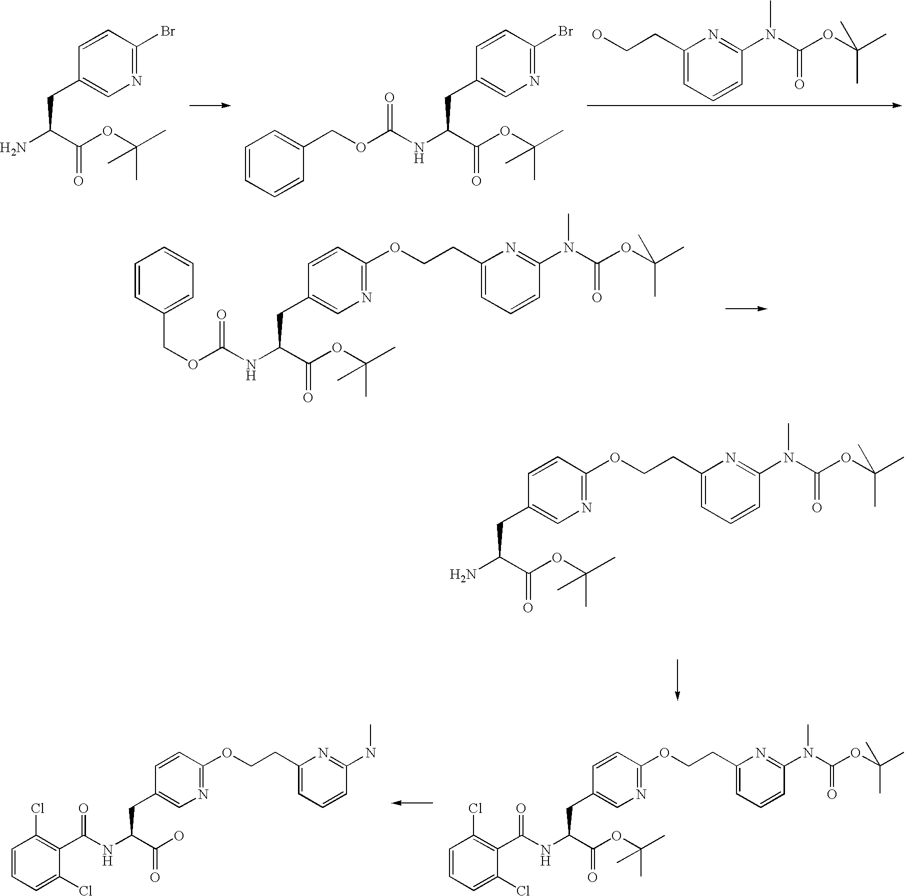

A solution of tert-butyl 3-[6-(2-{6-[(tert-butoxycarbonyl)(methyl)amino]pyridin-2-yl}ethoxy)pyridin-3-yl]-N-[(2,6-dichlorophenyl)carbonyl]-L-alaninate (200 mg, 0.31 mmol) in TFA (5 ml) was stirred at ambient temperature for 1 hour. After evaporation to dryness, the residue was redissolved in DMSO (1.6 ml) and 30% NH₄OH (0.4 ml) and purified by C18 reverse phase chromatography (basic conditions) to afford the title compound (113 mg, 74%); Mass spectrum [M+H]⁺=489; ¹H NMR Spectrum (DMSO-d6) 2.74 (d, 3H), 2.89 (dd, 1H), 2.93 (t 2H), 3.08 (dd, 1H), 4.46-4.55 (m, 3H), 6.26 (d, 1H), 6.35 (q, 1H), 6.40 (d, 1H), 6.65 (d, 1H), 7.29 (dd, 1H), 7.38 (dd, 1H), 7.40-7.45 (m, 2H), 7.60 (dd, 1H), 8.01 (d, 1H), 8.58 (bs, 1H).

The starting material was prepared as follows:

To a solution of tert-butyl 3-(6-bromopyridin-3-yl)-L-alaninate (1 g. 3.32 mmol) (WO2002/074761 page 38) in DMF (5 ml) was added TEA (0.93 ml, 6.64 mmol) and benzylchloroformate (0.5 ml, 3.49 mmol); The mixture was stirred for 3 hours, washed with NaHCO₃ and extracted with DCM. After evaporation, the residue was purified by flash chromatography eluting with 25% AcOEt in petroleum ether to give tert-butyl N-[(benzyloxy)carbonyl]-3-(6-bromopyridin-3-yl)-L-alaninate as an oil. (0.853 g, 59%) (containing about 10% of the D-enantiomer); ¹H NMR Spectrum (DMSOd6) 1.35 (s, 9H), 2.85 (dd, 1H), 3.01 (dd, 1H), 4.18 (ddd, 1H), 4.99 (s, 2H), 7.25-7.37 (m, 5H), 7.56 (d, 1H), 7.66 (dd, 1H), 7.77 (d, 1H), 8.26 (d, 1H).

A solution of tert-butyl N-[(benzyloxy)carbonyl]-3-(6-bromopyridin-3-yl)-L-alaninate (4.3 g, 9.88 mmol), tert-Butyl [6-(2-hydroxyethyl)pyridin-2-yl]methylcarbamate (described in Example 19) (5 g, 19.8 mmol), cesium carbonate (9.62 g, 29.6 mmol), palladium acetate (177 mg, 0.79 mmol) and 2-(di-tert-butylphosphino)-1,1′-binaphthyl (395 mg, 0.99 mmol) in toluene (100 ml) was degassed and heated at reflux for 17 hours under argon atmosphere. After concentration, a purification by silica gel flash chromatography (20 to 35% ethyl acetate in petroleum ether) afforded tert-butyl N-[(benzyloxy)carbonyl]-3-[6-(2-{6-[(tert-butoxycarbonyl)(methyl)amino]pyridin-2-yl}ethoxy)pyridin-3-yl]-L-alaninate (1.92 g, 32%); ¹H NMR Spectrum (CDCl3) 1.41 (s, 9H), 1.51 (s, 9H), 2.94 (dd, 1H), 3.04 (dd, 1H), 3.18 (t, 2H), 3.37 (s, 3H), 4.48 (dd, 1H), 4.63 (t, 2H), 5.09 (s, 2H), 5.29 (d, 1H), 6.61 (d, 1H), 6.93 (d, 1H), 7.28-7.40 (m, 5H), 7.49 (d, 1H), 7.53 (dd, 1H), 7.91 (d, 1H).

A mixture of tert-butyl N-[(benzyloxy)carbonyl]-3-[6-(2-{6-[(tert-butoxycarbonyl)(methyl)amino]pyridin-2-yl}ethoxy)pyridin-3-yl]-L-alaninate (1.92 g, 3.16 mmol) and Pd/C 10% (400 mg) in ethanol (150 ml) was hydrogenated under 3 bar of H₂ for 2 hours, filtered through celite, evaporated and purified by silica gel flash chromatography (40% AcOEt in petroleum ether) to afford tert-butyl 3-[6-(2-{6-[(tert-butoxycarbonyl)(methyl)amino]pyridin-2-yl}ethoxy)pyridin-3-yl]-L-alaninate (1.37 g, 92%); ¹H NMR Spectrum (CDCl3) 1.42 (s, 9H), 1.51 (s, 9H), 2.01 (bs, 2H), 2.88 (dd, 1H), 2.97 (dd, 1H), 3.19 (t, 2H), 3.37 (s, 3H), 3.62 (bs, 1H), 4.64 (t, 2H), 6.66 (d, 1H), 6.93 (d, 1H), 7.43-7.56 (m, 3H), 7.99 (s, 1H).

To a solution of tert-butyl 3-[6-(2-{6-[(tert-butoxycarbonyl)(methyl)amino]pyridin-2-yl}ethoxy)pyridin-3-yl]-L-alaninate (200 mg, 0.42 mmol) in DCM (5 ml) at 0° C. were added TEA (0.12 ml, 0.85 mmol) and 2,6-dichlorobenzoyl chloride (0.06 ml, 0.44 mmol); The mixture was stirred at 0° C. for 2 hours and purified by flash chromatography eluting with 40% ethyl acetate in petroleum ether to give tert-butyl 3-[6-(2-{6-[(tert-butoxycarbonyl)(methyl)amino]pyridin-2-yl}ethoxy)pyridin-3-yl]-N-[(2,6-dichlorophenyl)carbonyl]-L-alaninate as a foam (250 mg, 92%); Mass spectrum [M+H]⁺=646; ¹H NMR Spectrum (CDCl3) 1.42 (s, 9H), 1.51 (s, 9H), 3.11-3.22 (m, 4H), 3.37 (s, 3H), 4.63 (t, 2H), 5.00 (ddd, 1H), 6.34 (d, 1H), 6.64 (d, 1H), 6.92 (d, 1H), 7.26 (dd partially hidden br CHCl3, 1H), 7.29-7.34 (m, 2H), 7.46-7.56 (m, 3H), 8.00 (d, 1H).

### Example 55.1

### N-[(2-chloro-4-fluorophenyl)carbonyl]-3-(6-{2-[6-(methylamino)pyridin-2-yl]ethoxy}pyridin-3-yl)-L-alanine

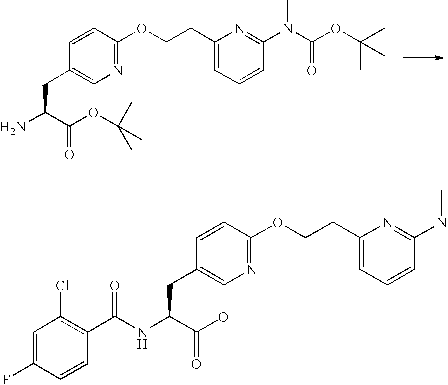

To a solution of tert-butyl 3-[6-(2-{6-[(tert-butoxycarbonyl)(methyl)amino]pyridin-2-yl}ethoxy)pyridin-3-yl]-L-alaninate (250 mg, 0.53 mmol) in DCM (1.5 ml) were added HOPO (77 mg, 0.69 mmol), EDCI (132 mg, 0.69 mmol) and 2-chloro-4-fluorobenzoic acid (92 mg, 0.53 mmol). The mixture was stirred at ambient temperature for 18 hours. Water (3 ml) was added The organic phase was separated and purified by flash chromatography eluting with 40% ethyl acetate in petroleum ether to give an intermediate which was redissolved in TFA (4 ml) and stirred at ambient temperature for 90 minutes. After evaporation to dryness, the residue was dissolved in DMSO (1.5 ml) and 30% NH₄OH (0.5 ml) and purified by C18 reverse phase chromatography (basic conditions) to afford the title compound (132 mg, 53%).

The procedure described above for Example 55.1 was repeated by coupling tert-butyl 3-[6-(2-{6-[(tert-butoxycarbonyl)(methyl)amino]pyridin-2-yl}ethoxy)pyridin-3-yl]-L-alaninate with the appropriate acid shown in Table 14 followed by removal of the protecting groups. Thus were obtained the compounds shown in Table 14:

TABLE 14

| | | | | Mass | |
|---|---|---|---|---|---|
| | | | | Ion | ¹H NMR Data (500 MHz |
| Example | acid | Name | Yield % | [M + H]⁺ | DMSOd6) |
| 55.1 | 2-chloro- | N-[(2-chloro- | 53 | 473 | 2.73 (d, 3H), |
| | 4- | 4- | | | 2.89 (dd, 1H), 2.93 (t, |
| | fluorobenzoic | fluorophenyl)carbonyl]- | | | 2H), 3.13 (dd, 1H), |
| | acid | 3-(6- | | | 4.43 (ddd, 1H), |
| | | {2-[6- | | | 4.52 (t, 2H), 6.25 (d, 1H), |
| | | (methylamino) | | | 6.35 (q, 1H), |
| | | pyridin-2- | | | 6.39 (d, 1H), 6.66 (d, |
| | | yl]ethoxy}pyridin- | | | 1H), 7.26 (ddd, 1H), |
| | | 3-yl)-L- | | | 7.29 (dd, 1H), |
| | | alanine | | | 7.36 (dd, 1H), 7.45 (dd, |
| | | | | | 1H), 7.58 (dd, 1H), |
| | | | | | 8.00 (d, 1H), |
| | | | | | 8.39 (d, 1H) |
| 55.2 | 2-chloro- | N-[(2-chloro- | 54 | 485 | 2.73 (d, 3H), |
| | 4- | 4- | | | 2.91 (dd, 1H), 2.93 (t, |
| | methoxy | methoxyphenyl) | | | 2H), 3.11 (dd, 1H), |
| | benzoic | carbonyl]-3- | | | 3.78 (s, 3H), |
| | acid | (6-{2-[6- | | | 4.42 (ddd, 1H), 4.52 (t, |
| | | (methylamino) | | | 2H), 6.25 (d, 1H), |
| | | pyridin-2- | | | 6.35 (q, 1H), |
| | | yl]ethoxy}pyridin- | | | 6.39 (d, 1H), 6.66 (d, |
| | | 3-yl)-L- | | | 1H), 6.94 (dd, 1H), |
| | | alanine | | | 7.01 (d, 1H), |
| | | | | | 7.26-7.32 (m, 2H), |
| | | | | | 7.57 (dd, 1H), 7.99 (d, |
| | | | | | 1H), 8.28 (d, 1H) |
| 55.3 | 2-ethyl- | N-[(2-ethyl-4- | 49 | 467 | 0.97 (t, 3H), 2.50 (q |
| | 4- | fluorophenyl)carbonyl]- | | | partially hidden by |
| | fluorobenzoic | 3-(6- | | | DMSOd5, 2H), |
| | acid | {2-[6- | | | 2.73 (d, 3H), 2.86 (dd, |
| | | (methylamino) | | | 1H), 2.93 (t, 2H), |
| | | pyridin-2- | | | 3.13 (dd, 1H), |
| | | yl]ethoxy}pyridin- | | | 4.49-4.56 (m, 3H), |
| | | 3-yl)-L- | | | 6.25 (d, 1H), 6.35 (q, |
| | | alanine | | | 1H), 6.39 (d, 1H), |
| | | | | | 6.69 (d, 1H), |
| | | | | | 7.04 (ddd, 1H), 7.07 (dd, |
| | | | | | 1H), 7.18 (dd, 1H), |
| | | | | | 7.29 (dd, 1H), |
| | | | | | 7.61 (dd, 1H), 8.02 (d, |
| | | | | | 1H), 8.44 (d, 1H) |

### Example 56

### N-[(2,6-dichlorophenyl)carbonyl]-3-(6-{3-[6-(methylamino)pyridin-2-yl]propyl}pyridin-3-yl)-L-alanine

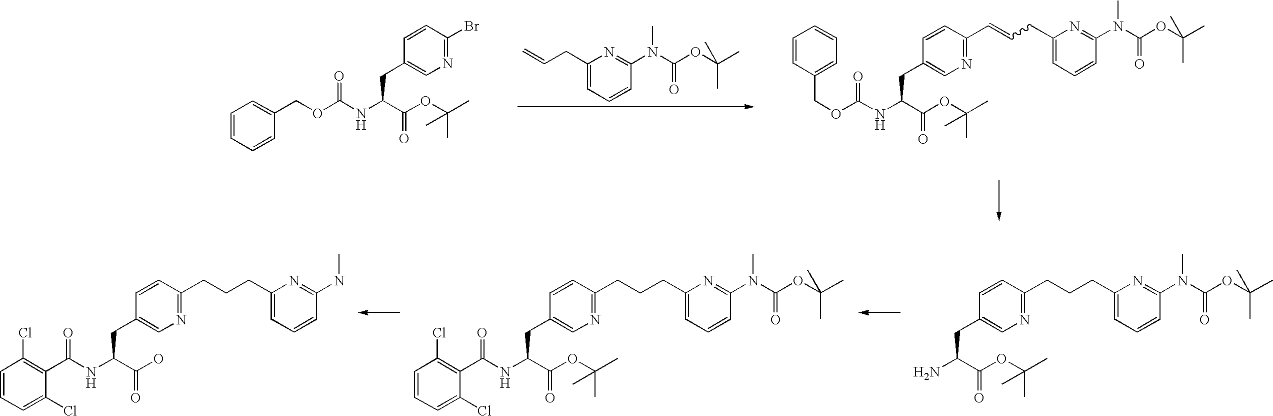

A solution of tert-butyl 3-[6-(3-{6-[(tert-butoxycarbonyl)(methyl)amino]pyridin-2-yl}propyl)pyridin-3-yl]-N-[(2,6-dichlorophenyl)carbonyl]-L-alaninate (40 mg, 0.06 mmol) in TFA (2 ml) was stirred at ambient temperature for 1 hour. After evaporation to dryness, the residue was redissolved in DMSO (1.6 ml) and 30% NH₄OH (0.4 ml) and purified by 10C18 reverse phase chromatography (basic conditions) to afford the title compound (23 mg, 74%); Mass spectrum [M+H]⁺=488; ¹H NMR Spectrum (DMSO-d6) 2.52 (t partially hidden by DMSOd5, 2H), 2.69 (t, 2H), 2.74 (d, 3H), 2.91 (dd, 1H), 3.14 (dd, 1H), 4.62 (ddd, 1H), 6.22 (d, 1H), 6.30 (q, 1H), 6.33 (d, 1H), 7.15 (d, 1H), 7.28 (dd, 1H), 7.37 (dd, 1H), 7.40-7.44 (m, 2H), 7.61 (dd, 1H), 8.36 (d, 1H), 8.84 (bs, 1H).

The starting material was prepared as follows:

A solution of tert-butyl N-[(benzyloxy)carbonyl]-3-(6-bromopyridin-3-yl)-L-alaninate (1.8 mg, 4.13 mmol) (described in Example 54), tri-O-tolylphosphine (125 mg, 0.41 mmol), allylpalladium chloride (77 mg, 0.21 mmol), sodium acetate (1.02 g, 12.4 mmol) and tert-butyl (6-allylpyridin-2-yl)methylcarbamate (described in Example 18) (3.01 g, 8.27 mmol) in dioxan (10 ml) was degassed, then sealed and heated to 150° C. overnight. After evaporation to dryness, the residue was purified by flash chromatography eluting with 10% ether in petroleum ether to give tert-butyl N-[(benzyloxy)carbonyl]-3-{6-[(IE)-3-{6-[(tert-butoxycarbonyl)(methyl)amino]pyridin-2-yl}prop-1-en-1-yl]pyridin-3-yl}-L-alaninate as a gum (87 mg, 3%).

A mixture of tert-butyl N-[(benzyloxy)carbonyl]-3-{6-[(IE)-3-{6-[(tert-butoxycarbonyl)(methyl)amino]pyridin-2-yl}prop-1-en-1-yl]pyridin-3-yl}-L-alaninate (117 mg, 0.19 mmol) and Pd/C 10% (80 mg) in ethanol (15 ml) was hydrogenated under 4 bar of H₂ for 4 hours, filtered through celite, evaporated and purified by silica gel flash chromatography (1 to 4% methanol in DCM) to afford tert-butyl 3-[6-(3-{6-[(tert-butoxycarbonyl)(methyl)amino]pyridin-2-yl}propyl)pyridin-3-yl]-L-alaninate (48 mg, 53%); Mass spectrum [M+H]⁺=471; ¹H NMR Spectrum (CDCl3) 1.42 (s, 9H), 1.50 (s, 9H), 1.84 (bs, 2H), 2.11-2.21 (m, 2H), 2.79 (t, 2H), 2.86 (t, 2H), 2.89 (dd, 1H), 3.01 (dd, 1H), 3.38 (s, 3H), 3.63 (dd, 1H), 6.95 (d, 1H), 7.15 (d, 1H), 7.42 (d, 1H), 7.50 (d, 1H), 7.54 (d, 1H), 8.42 (s, 1H).

To a solution of tert-butyl 3-[6-(3-{6-[(tert-butoxycarbonyl)(methyl)amino]pyridin-2-yl}propyl)pyridin-3-yl]-L-alaninate (48 mg, 0.10 mmol) in DCM (5 ml) were added at 0° C. TEA (0.03 ml, 0.5 mmol) and 2,6-dichlorobenzoyl chloride (0.02 ml, 0.11 mmol). The mixture was stirred at 0° C. for 1 hour. 10% citric acid was added and the organic phase was separated and purified by flash chromatography eluting with 1-4% MeOH in DCM to give tert-butyl 3-[6-(3-{6-[(tert-butoxycarbonyl)(methyl)amino]pyridin-2-yl}propyl)pyridin-3-yl]-N-[(2,6-dichlorophenyl)carbonyl]-L-alaninate as a foam (40 mg, 61%); Mass spectrum [M+H]⁺=643; ¹H NMR Spectrum (CDCl3) 1.42 (s, 9H), 1.51 (s, 9H), 2.12-2.21 (m, 2H), 2.78 (t, 2H), 2.81-2.95 (m, 2H), 4.24 (d, 2H), 3.39 (s, 3H), 5.03 (dd, 1H), 6.40 (d, 1H), 6.84 (d, 1H), 7.18 (bs, 1H), 7.27 dd partially hidden by CHCl3, 1H), 7.29-7.34 (m, 2H), 7.42 (d, 1H), 7.50 (dd, 1H), 7.68 (bs, 1H), 8.42 (s, 1H).

### Example 57

### N-[(2,6-dichlorophenyl)carbonyl]-3-(4-{3-[6-(methylamino)pyridin-2-yl]propyl}thiophen-2-yl)alanine (racemic mixture)

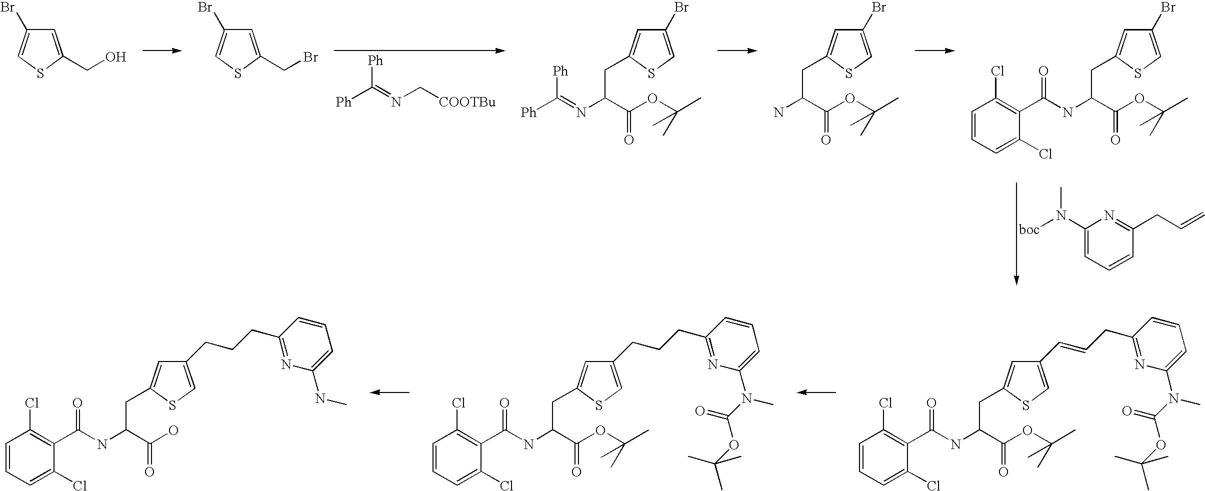

To a solution of tert-butyl 3-[4-(3-{6-[(tert-butoxycarbonyl)(methyl)amino]pyridin-2-yl}propyl)thiophen-2-yl]-N-[(2,6-dichlorophenyl)carbonyl]alaninate (330 mg, 0.51 mmol) in DCM (4 ml) was added TFA (5 ml). The reaction mixture was stirred at room temperature overnight. After addition of saturated bicarbonate, the mixture was extracted in DCM. The organic phase was evaporated and purified by C18 reverse phase chromatography (basic conditions) to afford the title compound as a white solid (150 mg, 60%); Mass Spectrum [M+H]⁺=493; ¹H NMR Spectrum (DMSOD6) 1.84-1.93 (m, 2H), 2.94 (t, partially hidden by DMSOd5, 2H), 2.52 (t partially hidden by DMSOd5, 2H), 2.74 (d, 3H), 3.14 (dd, 1H), 3.26 (dd, 1H), 4.57 (ddd, 1H), 6.22 (d, 1H), 6.28-6.35 (m, 2H), 6.81 (s, 1H), 6.93 (s, 1H), 7.28 (dd, 1H), 7.39 (dd, 1H), 7.42-7.46 (m, 2H), 8.93 (bs, 1H).

The starting material was prepared as follows:

To a solution of 4-bromo-2-hydroxymethylthiophene (12.25 g, 63.45 mol) in DCM (220 ml) was added at 0° C. PBr₃ (18.89 g, 69.8 mmol). The reaction mixture was stirred at 0° C. for 3 hours neutralised with sodium bicarbonate and extracted with DCM. The organic phase was evaporated and purified by silica gel flash chromatography (5% ethyl acetate in petroleum ether) to afford 4-bromo-2-bromomethylthiophene (11.35 g, 70%); Mass Spectrum [M+H]⁺=257; ¹H NMR Spectrum (CDCl3) 4.79 (s, 2H), 6.93 (d, 1H), 7.18 (d, 1H)

To a solution of tert-butyl N-(diphenylmethylidene)glycinate (1.62 g, 5.48 mmol) and 4-bromo-2-bromomethylthiophene (168 g, 6.58 mol) in DCM (17 ml) was added tetrabutylammonium sulfate (2.23 g, 6.58 mmol) and 10% NaOH (7.8 ml, 19.2 mmol). The reaction mixture was stirred at room temperature for 3 hours and extracted with ethyl acetate. The organic phase was evaporated and purified by silica gel flash chromatography (0 to 10% ethyl acetate in petroleum ether) to afford tert-butyl 3-(4-bromothiophen-2-yl)-N-(diphenylmethylidene)alaninate (2.46 g, 95%); Mass spectrum [M+H]⁺=471; ¹H NMR Spectrum (CDCl3) 1.46 (s, 9H), 3.29-3.42 (m, 2H), 4.11 (dd, 1H), 6.64 (s, 1H), 6.88 (d, 1H), 7.30-7.42 (m, 6H), 7.64 (d, 2H).

To a solution of tert-butyl 3-(4-bromothiophen-2-yl)-N-(diphenylmethylidene) alaninate (2.65 g, 5.63 mmol) in ether (20 ml) was added 2N HCl (8 ml). The reaction mixture was stirred at room temperature overnight. The solution was neutralised with 0.2N NaOH and extracted with ethyl acetate. The organic phase was evaporated and purified by silica gel flash chromatography (50 to 10% ethyl acetate in petroleum ether) to afford tert-butyl 3-(4-bromothiophen-2-yl)alaninate (1.49 g, 87%); ¹H NMR Spectrum (CDCl3) 1.46 (s, 9H), 1.70 (bs, 2H), 3.07 (dd, 1H), 3.19 (dd, 1H), 3.59 (dd, 1H), 6.81 (bs, 1H), 7.07 (d, 1H).

To a solution of tert-butyl 3-(4-bromothiophen-2-yl)alaninate (500 mg, 1.63 mmol) in DCM (15 ml) was added at 0° C. TEA (0.455 ml, 3.27 mmol) and 2,6 dichlorobenzoic acid (341 mg, 1.63 mmol). The reaction mixture was stirred at room temperature for 5 hours and extracted with ethyl acetate. The organic phase was evaporated and purified by silica gel flash chromatography (10 to 25% ethyl acetate in petroleum ether) to afford tert-butyl 3-(4-bromothiophen-2-yl)-N-[(2,6-dichlorophenyl)carbonyl]alaninate (653 mg, 84%); Mass spectrum [M+H]⁺=479; ¹H NMR Spectrum (CDCl₃) 1.46 (s, 9H), 3.46 (d, 2H), 5.00 (ddd, 1H), 6.46 (d, 1H), 6.85 (bs, 1H), 7.06 (d, 1H), 7.27 (dd, 1H), 7.31-7.35 (m, 2H).

A solution of tert-butyl 3-(4-bromothiophen-2-yl)-N-[(2,6-dichlorophenyl)carbonyl]alaninate (590 mg, 1.23 mmol), tri-O-tolylphosphine (149 mg, 0.49 mmol), palladium acetate (83 mg, 0.37 mmol) and tert-butyl (6-allylpyridin-2-yl)methylcarbamate (305 mg, 1.23 mmol) described in example 18 in acetonitrile (5 ml) was degassed and heated at 80° C. for 4 hours. After evaporation to dryness, the residue was purified by flash chromatography eluting with 10 to 25% ethyl acetate in petroleum ether to give tert-butyl 3-{4-[(1E)-3-{6-[(tert-butoxycarbonyl)(methyl)amino]pyridin-2-yl}prop-1-en-1-yl]thiophen-2-yl}-N-[(2,6-dichlorophenyl)carbonyl]alaninate as a foam (462 mg, 58%); Mass spectrum [M+H]⁺=647.

A mixture of tert-butyl 3-{4-[(1E)-3-{6-[(tert-butoxycarbonyl)(methyl)amino]pyridin-2-yl}prop-1-en-1-yl]thiophen-2-yl}-N-[(2,6-dichlorophenyl)carbonyl]alaninate (265 mg, 0.41 mmol) and Pd/C 10% (60 mg) in ethanol (14 ml) and ethyl acetate (4 ml) was hydrogenated under 4 bar of H₂ for 4 hours, filtered through celite, evaporated and purified by silica gel flash chromatography (20 to 30% AcOEt in petroleum ether) to afford tert-butyl 3-[4-(3-{6-[(tert-butoxycarbonyl)(methyl)amino]pyridin-2-yl}propyl)thiophen-2-yl]-N-[(2,6-dichlorophenyl)carbonyl]alaninate (207 mg, 78%); ¹H NMR Spectrum (CDCl3) 1.44 (s, 9H), 1.51 (s, 9H), 1.95-2.04 (d, 2H), 2.58 (t, 2H), 2.72 (t, 2H), 3.38 (s, 3H), 3.44 (t, 2H), 5.02 (ddd, 1H), 6.43 (d, 1H), 6.76 (d, 2H), 6.81 (d, 1H), 7.24 (dd, 1H), 7.28-7.32 (m, 2H), 7.43 (d, 1H), 7.51 (dd, 1H).

### Example 58

### N-[(2,6-dichlorophenyl)carbonyl]-3-(4-{2-[6-(methylamino)pyridin-2-yl]ethoxy}thiophen-2-yl)alanine (racemic mixture)

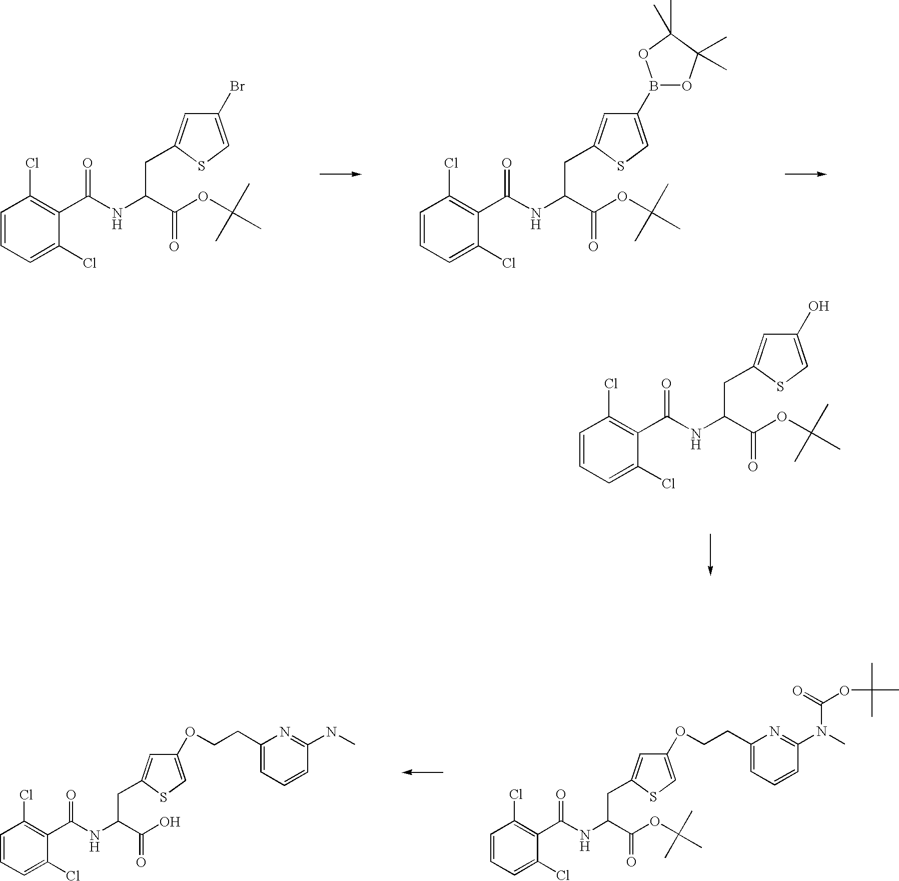

To a solution of tert-butyl 3-[4-(2-{6-[(tert-butoxycarbonyl)(methyl)amino]pyridin-2-yl}ethoxy)thiophen-2-yl]-N-[(2,6-dichlorophenyl)carbonyl]alaninate (100 mg, 0.15 mmol, described in Example 57) in DCM (0.3 ml) was added 4N HCl in dioxan (1 ml). The mixture was stirred at ambient temperature overnight. After evaporation to dryness, the residue was redissolved in DCM (1.6 ml) and 30% NH₄OH (0.4 ml) and purified by C18 reverse phase chromatography (acidic conditions) to afford the title compound (28 mg, 37%); Mass spectrum [M+H]⁺=494; ¹H NMR Spectrum (DMSO-d6) 2.74 (d, 3H), 2.91 (t, 2H), 3.07 (dd, 1H), 3.19 (dd, 1H), 4.19 (t, 2H), 4.36 (bs, 1H), 6.26 (d, 1H), 6.35 (q, 1H), 6.38 (s, 1H), 6.41 (d, 1H), 6.59 (s, 1H), 7.30 (dd, 1H), 7.40 (dd, 1H), 7.43-7.47 (m, 2H), 9.10 (s, 1H).

The starting material was prepared as follows:

To a solution of tert-butyl N-[(2,6-dichlorophenyl)carbonyl]-3-(4-bromothiophen-2-yl)alaninate (800 mg, 1.67 mmol) in dioxan (10 ml) was added bis(pinacolato)diboron (551 mg, 2.17 mmol), PdCl₂dppf (82 mg, 0.1 mmol) and potassium acetate (492 mg, 5.01 mmol). The mixture was heated at 85° C. under argon atmosphere for 8 hours. After extraction with DCM and evaporation, the residue was purified by silica gel flash chromatography (0 to 20% AcOEt in petroleum ether) to afford tert-butyl N-[(2,6-dichlorophenyl)carbonyl]-3-[4-(4,4,5,5-tetramethyl-1,3,2-dioxaborolan-2-yl)thiophen-2-yl]alaninate as an impure material which was used in the next step (800 mg).

To a solution of tert-butyl N-[(2,6-dichlorophenyl)carbonyl]-3-[4-(4,4,5,5-tetramethyl-1,3,2-dioxaborolan-2-yl)thiophen-2-yl]alaninate (740 mg, 1.41 mmol) in ethanol (6 ml) were added at 0° C. 30% H₂O₂ (319 μl, 2.81 mmol). The mixture was stirred at ambient temperature for 3 hours. A solution of sodium thiosulfate (697 mg, 2.81 mmol) in water (10 ml) was added at 0° C. The reaction mixture was extracted with ethyl acetate, evaporated and purified by silica gel flash chromatography (20 to 50% AcOEt in petroleum ether) to afford tert-butyl N-[(2,6-dichlorophenyl)carbonyl]-3-(4-hydroxythiophen-2-yl)alaninate as a gum (402 mg, 69%); Mass spectrum [M+H]⁺=416; ¹H NMR Spectrum (DMSOd6) 1.40 (s, 9H), 3.01 (dd, 1H), 3.12 (dd, 1H), 4.55 (ddd, 1H), 6.05 (d, 1H), 6.53 (d, 1H), 7.42 (dd, 1H), 7.45-7.59 (m, 1H), 9.16 (d, 1H), 9.32 (s, 1H).

DTAD (336 mg, 1.46 mmol) was added to a stirred solution of tert-butyl N-[(2,6-dichlorophenyl)carbonyl]-3-(4-hydroxythiophen-2-yl)alaninate (405 mg, 0.97 mmol), tert-Butyl [6-(2-hydroxyethyl)pyridin-2-yl]methylcarbamate (295 mg, 1.17 mmol) and PPh₃ (383 mg, 1.46 mmol) in THF (5 ml). The reaction mixture was stirred at ambient temperature overnight. After evaporation to dryness, the residue was purified by C18 reverse phase chromatography (acidic conditions) to afford tert-butyl 3-[4-(2-{6-[(tert-butoxycarbonyl)(methyl)amino]pyridin-2-yl}ethoxy)thiophen-2-yl]-N-[(2,6-dichlorophenyl)carbonyl]alaninate (202 mg, 32%); ¹H NMR Spectrum (DMSOd6) 1.38 (s, 9H), 1.45 (s, 9H), 3.03 (dd, 1H), 3.09 (t, 2H), 3.14 (dd, 1H), 3.28 (s, 3H), 4.26 (t, 2H), 4.57 (ddd, 1H), 6.43 (d, 1H), 6.60 (s, 1H), 7.06 (d, 1H), 7.41 (dd, 1H), 7.44-7.48 (m, 3H), 7.67 (dd, 1H), 9.15 (d, 1H).

### Example 59.1

### N-[(2,6-dimethylphenyl)carbonyl]-3-(5-{3-[6-(methylamino)pyridin-2-yl]propyl}thiophen-2-yl)-L-alanine

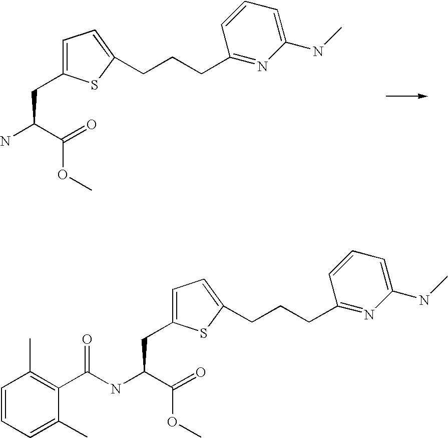

To a solution of 2,6-dimethylbenzoic acid (86 mg, 0.57 mmol) in DMF (1.5 ml) were added TBTU (259 mg, 0.68 mmol) and methyl 3-(5-{3-[6-(methylamino)pyridin-2-yl]propyl}-2-thienyl)-L-alaninate (190 mg, 0.57 mmol, described in Example 29). The mixture was stirred at ambient temperature for 48 hours. NaOH 6N (15 drops) was added. After stirring for 3 hours, the mixture was purified by C18 reverse phase chromatography (basic conditions) to afford the title compound (154 mg, 59%); Mass spectrum [M+H]⁺=452; ¹H NMR Spectrum (DMSOD6) 1.87-1.95 (m, 2H), 2.09 (s, 6H), 2.54 (t, 2H), 2.72 (t, 2H), 2.74 (d, 3H), 3.09 (dd, 1H), 3.30 (dd, 1H), 4.34-4.41 (m, 1H), 6.22 (d, 1H), 7.30 (q, 1H), 7.31 (d, 1H), 6.59 (d, 1H), 6.66 (d, 1H), 6.95 (d, 1H), 7.05 (d, 1H), 7.10 (dd, 1H), 7.27 (dd, 1H), 7.82 (bs, 1H).

### Example 59.2

### N-[(2-chloro-6-fluorophenyl)carbonyl]-3-(5-{3-[6-(methylamino)pyridin-2-yl]propyl}thiophen-2-yl)-L-alanine

The title compound was obtained using the same procedure as for example 59.1 but using 2-chloro-6-fluorobenzoic acid (Yield 70%); Mass spectrum [M+H]⁺=477; ¹H NMR Spectrum (DMSOD6) 1.87-1.96 (m, 2H), 2.54 (t, 2H), 2.73 (t, 2H), 2.74 (d, 3H), 3.11 (dd, 1H), 3.28 (dd, 1H), 4.49 (ddd, 1H), 6.22 (d, 1H), 6.26-6.35 (m, 2H), 6.62 (d, 1H), 6.70 (d, 1H), 7.21-7.33 (m, 3H), 7.41-7.47 (m, 1H), 8.80 (d, 1H).

### Example 60.1

### O-[2-(3,4-dihydro-2H-pyrido[3,2-b][1,4]oxazin-6-yl)ethyl]-N-[(2,6-dimethylphenyl)carbonyl]-L-tyrosine

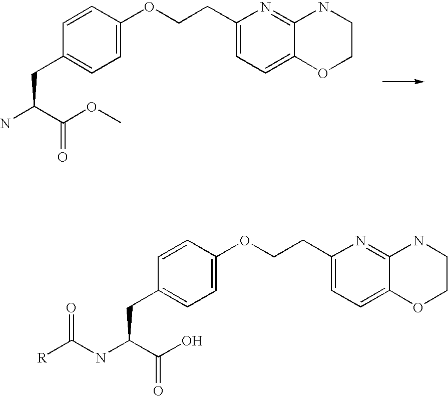

To a solution of 2,6-dimethylbenzoic acid (47 mg, 0.31 mmol) in DMF (1 ml) were added TBTU (116 mg, 0.36 mmol), N-methylmorpholine (34 μl, 0.31 mmol) and methyl O-[2-(3,4-dihydro-2H-pyrido[3,2-b][1,4]oxazin-6-yl)ethyl]-L-tyrosinate (100 mg, 0.28 mmol, described in example 28.1). The mixture was stirred at ambient temperature for 48 hours. NaOH 6N (200 μl) was added. After stirring for 4 hours at 40° C., the mixture was purified by C18 reverse phase chromatography (basic conditions) to afford the title compound (74 mg, 56%).

The procedure described above for example 60.1 was repeated by coupling methyl O-[2-(3,4-dihydro-2H-pyrido[3,2-b][1,4]oxazin-6-yl)ethyl]-L-tyrosinate using the appropriate acids shown in Table 15. Thus were obtained the compounds shown in Table 15:

TABLE 15

| | | | | Mass | |
|---|---|---|---|---|---|
| | | | | Ion | ¹H NMR Data (500 MHz |
| Example | acid | Name | Yield % | [M + H]⁺ | DMSOd6) |
| 60.1 | 2,6- | O-[2-(3,4- | 56 | 476 | 1.98 (s, 6H), |
| | dimethylbenzoic | dihydro-2H- | | | 2.80 (dd, 1H), 2.88 (t, |
| | acid | pyrido[3,2- | | | 2H), 3.11 (dd, 1H), |
| | | b][1,4]oxazin- | | | 3.38 (m partially |
| | | 6-yl)ethyl]-N- | | | hidden by H2O, 2H), |
| | | [(2,6- | | | 4.07 (t, 2H), 4.18 (t, |
| | | dimethylphenyl) | | | 2H), 4.60 (ddd, 1H), |
| | | carbonyl]-L- | | | 6.41 (d, 1H), |
| | | tyrosine | | | 6.65 (bs, 1H), 6.84 (d, |
| | | | | | 2H), 6.86 (d, 1H), |
| | | | | | 6.96 (d, 2H), 7.11 (t, |
| | | | | | 1H), 7.19 (d, 2H), |
| | | | | | 8.49 (bs, 1H) |
| 60.2 | 2-chloro- | N-[(2-chloro- | 65 | 497 | 2.07 (s, 3H), |
| | 6- | 6- | | | 2.84 (dd, 1H), 2.88 (t, |
| | methylbenzoic | methylphenyl) | | | 2H), 3.08 (dd, 1H), |
| | acid | carbonyl]-O- | | | 3.39 (m partially |
| | | [2-(3,4- | | | hidden by H2O, 2H), |
| | | dihydro-2H- | | | 4.08 (t, 2H), 4.19 (t, |
| | | pyrido[3,2- | | | 2H), 4.57 (ddd, 1H), |
| | | b][1,4]oxazin- | | | 6.41 (d, 1H), |
| | | 6-yl)ethyl]-L- | | | 6.65 (bs, 1H), 6.83 (d, |
| | | tyrosine | | | 2H), 6.86 (d, 1H), |
| | | | | | 7.15 (dd, 1H), |
| | | | | | 7.19 (d, 2H), |
| | | | | | 7.21-7.28 (m, 2H), 8.72 (ms, |
| | | | | | 1H) |
| 60.3 | 2-ethyl-4- | O-[2-(3,4- | 77 | 494 | 1.04 (t, 3H), 2.59 (q, |
| | fluorobenzoic | dihydro-2H- | | | 2H), 3.89 (t, 2H), |
| | acid | pyrido[3,2- | | | 3.94 (dd, 1H), |
| | | b][1,4]oxazin- | | | 3.14 (dd, 1H), 3.39 (bs, |
| | | 6-yl)ethyl]-N- | | | 2H), 4.08 (t, 2H), |
| | | [(2-ethyl-4- | | | 4.20 (t, 2H), |
| | | fluorophenyl)carbonyl]- | | | 4.37 (bs, 1H), 6.41 (d, |
| | | L- | | | 1H), 6.49 (bs, 1H), |
| | | tyrosine | | | 6.81 (d, 2H), 6.84 (d, |
| | | | | | 1H), 7.01 (ddd, 1H), |
| | | | | | 7.05 (dd, 1H), |
| | | | | | 7.15 (d, 2H), 7.19 (dd, |
| | | | | | 1H), 7.82 (d, 1H) |
| 60.4 | 2,4,6- | O-[2-(3,4- | 44 | 490 | 1.97 (s, 6H), 2.20 (s, |
| | trimethylbenzoic | dihydro-2H- | | | 3H), 2.84 (dd, 1H), |
| | acid | pyrido[3,2- | | | 2.88 (t, 2H), |
| | | b][1,4]oxazin- | | | 3.10 (dd, 1H), 3.37 (bs, |
| | | 6-yl)ethyl]-N- | | | 2H), 4.08 (t, 2H), |
| | | [(2,4,6- | | | 4.17 (t, 2H), |
| | | trimethylphenyl) | | | 4.42 (ddd, 1H), 6.41 (d, |
| | | carbonyl]-L- | | | 1H), 6.66 (bs, 1H), |
| | | tyrosine | | | 6.76 (s, 2H), 6.79 (d, |
| | | | | | 2H), 6.85 (d, 1H), |
| | | | | | 7.15 (d, 2H), 7.88 (d, |
| | | | | | 1H) |
| 60.5 | 2-chloro- | N-[(2-chloro- | 76 | 513 | 2.87 (t, 2H), |
| | 4- | 4- | | | 2.95 (dd, 1H), 3.11 (dd, |
| | methoxybenzoic | methoxyphenyl) | | | 1H), 3.36 (m |
| | acid | carbonyl]-O- | | | partially hidden by |
| | | [2-(3,4- | | | H2O, 2H), 3.79 (s, |
| | | dihydro-2H- | | | 3H), 4.07 (dd, 2H), |
| | | pyrido[3,2- | | | 4.17 (t, 2H), |
| | | b][1,4]oxazin- | | | 4.21 (dd, 1H), 6.41 (d, |
| | | 6-yl)ethyl]-L- | | | 1H), 6.66 (bs, 1H), |
| | | tyrosine | | | 6.77 (d, 2H), 6.85 (d, |
| | | | | | 1H), 6.94 (dd, 1H), |
| | | | | | 7.02 (d, 1H), 7.10 (d, |
| | | | | | 2H), 7.32 (d, 1H), |
| | | | | | 7.83 (bs, 1H) |
| 60.6 | 2-chloro- | N-[(2-chloro- | 82 | 500 | 2.88 (t, 2H), |
| | 5- | 5- | | | 2.90 (dd, 1H), 3.11 (dd, |
| | fluorobenzoic | fluorophenyl)carbonyl]- | | | 1H), 3.37 (m |
| | acid | O-[2- | | | partially hidden by |
| | | (3,4-dihydro- | | | H2O, 2H), 4.07 (dd, |
| | | 2H-pyrido[3,2- | | | 2H), 4.18 (t, 2H), |
| | | b][1,4]oxazin- | | | 4.41 (dd, 1H), |
| | | 6-yl)ethyl]-L- | | | 6.41 (d, 1H), 6.65 (bs, |
| | | tyrosine | | | 1H), 6.82 (d, 2H), |
| | | | | | 6.85 (d, 1H), |
| | | | | | 7.05 (dd, 1H), 7.16 (d, |
| | | | | | 2H), 7.32 (ddd, 1H), |
| | | | | | 7.51 (dd, 1H), |
| | | | | | 8.48 (bs, 1H) |
| 60.7 | 2-ethyl-5- | O-[2-(3,4- | 77 | 494 | 1.92-2.03 (m, 2H), |
| | fluorobenzoic | dihydro-2H- | | | 0.98 (t, 3H), 2.49 (q |
| | acid | pyrido[3,2- | | | partially hidden by |
| | | b][1,4]oxazin- | | | DMSOd5, 2H), |
| | | 6-yl)ethyl]-N- | | | 2.83-2.91 (m, 3H), |
| | | [(2-ethyl-5- | | | 3.12 (dd, 1H), 3.38 (m |
| | | fluorophenyl)carbonyl]- | | | partially hidden by |
| | | L- | | | H2O, 2H), 4.07 (t, |
| | | tyrosine | | | 2H), 4.19 (t, 2H), |
| | | | | | 4.50 (ddd, 1H), |
| | | | | | 6.41 (d, 1H), 6.64 (bs, |
| | | | | | 1H), 6.81-6.89 (m, |
| | | | | | 4H), 7.15-7.21 (m, |
| | | | | | 3H), 7.27 (dd, 1H), |
| | | | | | 8.53 (bs, 1H) |

### Example 61.1

### N-[(2-chloro-6-fluorophenyl)carbonyl]-3-(5-{3-[6-(methylamino)pyridin-2-yl]propyl}pyridin-2-yl)-L-alanine

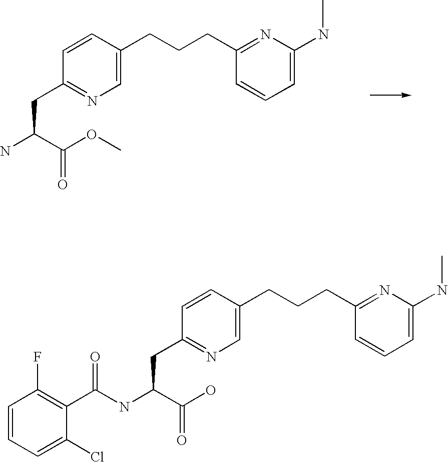

TBTU (190 mg, 0.5 mmol) was added last to a stirred solution of the methyl 3-(5-{3-[6-(methylamino)pyridin-2-yl]propyl}pyridin-2-yl)-L-alaninate. 3HCl (0.2 g, 0.46 mmol, described in Example 42.1), 2-chloro-6-fluorobenzoic acid (87 mg, 0.5 mmol) and N-methylmorpholine (22 μl, 2.02 mmol). The resulting solution was stirred at ambient temperature for 16 hours. The reaction mixture was treated with NaOH 6N (300 μl), stirred for 2 hours and purified by C18 reverse phase chromatography (basic conditions) to afford the title compound (11 mg, 50%).

The procedure described above for example 61.1 was repeated by coupling methyl 3-(5-{3-[6-(methylamino)pyridin-2-yl]propyl}pyridin-2-yl)-L-alaninate. 3HCl using the appropriate acids shown in Table 16. Thus were obtained the compounds shown in Table 16:

TABLE 16

| | | | | Mass | |
|---|---|---|---|---|---|
| | | | | Ion | ¹H NMR Data (500 MHz |
| Example | acid | Name | Yield % | [M + H]⁺ | DMSOd6) |
| 61.1 | 2-chloro- | N-[(2-chloro-6- | 50 | 471 | 1.86-1.96 (m, 2H), |
| | 6- | fluorophenyl)carbonyl]- | | | 2.52 (t partially |
| | fluorobenzoic | 3-(5-{3-[6- | | | hidden by DMSOd5, |
| | acid | (methylamino)pyridin- | | | 2H), 2.59 (t, 2H), |
| | | 2- | | | 2.74 (d, 3H), |
| | | yl]propyl}pyridin- | | | 3.03 (dd, 1H), 3.24 (dd, |
| | | 2-yl)-L-alanine | | | 1H), 4.80 (bs, 1H), |
| | | | | | 6.22 (d, 1H), 6.29 (q, |
| | | | | | 1H), 6.32 (d, 1H), |
| | | | | | 7.19-7.24 (m, 2H), |
| | | | | | 7.25-7.31 (m, 2H), |
| | | | | | 7.42 (ddd, 1H), |
| | | | | | 7.53 (dd, 1H), 8.34 (d, |
| | | | | | 1H), 8.88 (bs, 1H) |
| 61.2 | 2-chloro- | N-[(2-chloro-4- | 47 | 471 | 1.86-1.96 (m, 2H), |
| | 4- | fluorophenyl)carbonyl]- | | | 2.52 (t partially |
| | fluorobenzoic | 3-(5-{3-[6- | | | hidden by DMSOd5, |
| | acid | (methylamino)pyridin- | | | 2H), 2.59 (t, 2H), |
| | | 2- | | | 2.73 (d, 3H), |
| | | yl]propyl}pyridin- | | | 3.07 (dd, 1H), 3.26 (dd, |
| | | 2-yl)-L-alanine | | | 1H), 4.77 (bs, 1H), |
| | | | | | 6.22 (d, 1H), 6.29 (q, |
| | | | | | 1H), 6.31 (d, 1H), |
| | | | | | 7.22 (d, 1H), |
| | | | | | 7.24 (dd, 1H), 7.27 (dd, |
| | | | | | 1H), 7.32 (dd, 1H), |
| | | | | | 7.43 (dd, 1H), |
| | | | | | 7.56 (dd, 1H), 8.35 (d, |
| | | | | | 1H), 8.65 (bs, 1H) |
| 61.3 | 2-ethyl- | N-[(2-ethyl-4- | 54 | 465 | 0.95 (t, 3H), |
| | 4- | fluorophenyl)carbonyl]- | | | 1.87-1.95 (m, 2H), 2.48 (q |
| | fluorobenzoic | 3-(5-{3-[6- | | | partially hidden by |
| | acid | (methylamino)pyridin- | | | DMSOd5, 2H), |
| | | 2- | | | 2.52 (t partially hidden by |
| | | yl]propyl}pyridin- | | | DMSOd5, 2H), |
| | | 2-yl)-L-alanine | | | 2.59 (t, 2H), 2.73 (d, 3H), |
| | | | | | 3.07 (dd, 1H), |
| | | | | | 3.25 (dd, 1H), 4.77 (bs, |
| | | | | | 1H), 6.22 (d, 1H), |
| | | | | | 6.29 (q, 1H), 6.31 (d, |
| | | | | | 1H), 7.01 (ddd, 1H), |
| | | | | | 7.05 (dd, 1H), |
| | | | | | 7.17 (dd, 1H), 7.22 (d, |
| | | | | | 1H), 7.27 (dd, 1H), |
| | | | | | 7.56 (dd, 1H), |
| | | | | | 8.35 (d, 1H), 8.44 (bs, 1H) |

### Example 62.1

### N-[(2,6-dichlorophenyl)carbonyl]-3-{6-[2-(5,6,7,8-tetrahydro-1,8-naphthyridin-2-yl)ethoxy]pyridin-3-yl}-L-alanine

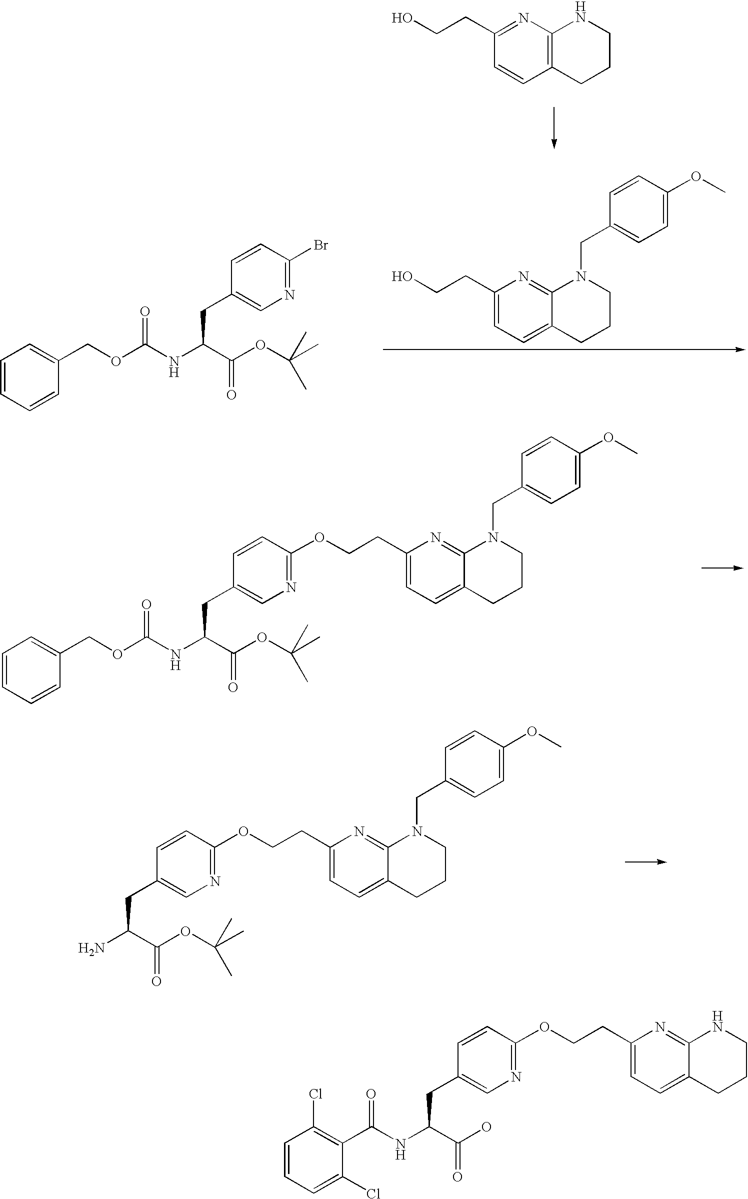

To a solution of tert-butyl 3-(6-{2-[8-(4-methoxybenzyl)-5,7,8-tetrahydro-1,8-naphthyridin-2-yl]ethoxy}pyridin-3-yl)-L-alaninate (250 mg, 0.48 mmol) in DCM (2 ml) were added HOPO (70 mg, 0.63 mmol), EDCI (121 mg, 0.63 mmol) and 2,6-dichlorobenzoic acid (92 mg, 0.48 mmol). The mixture was stirred at ambient temperature for 20 hours. Anisole (63 μl, 0.58 mmol) and TFA (2 ml) were added. The mixture was stirred at ambient temperature overnight. After evaporation to dryness, the residue was treated with DMF (1 ml) and NH₃ 7N in methanol (0.5 ml). The resulting solid was filtered, taken up in acetonitrile (3 ml) and water (6 ml) and treated with HCl 2N (230 μl). The solution was lyophilized to give the title compound as a solid (128 mg, 48%).

The procedure described above for example 60.1 was repeated by coupling tert-butyl 3-(6-{2-[8-(4-methoxybenzyl)-5,6,7,8-tetrahydro-1,8-naphthyridin-2-yl]ethoxy}pyridin-3-yl)-L-alaninate with the appropriate acids shown in Table 17. Thus were obtained the compounds shown in Table 17:

TABLE 17

| | | | | Mass | ¹H NMR Data (500 MHz |
|---|---|---|---|---|---|
| Example | acid | Name | Yield % | Ion[M + H]⁺ | DMSOd6) |
| 62.1 | 2,6- | N-[(2,6- | 48 | 515 | (DMSOd6 + TFAd) |
| | dichloro | dichlorophenyl) | | | 1.79-1.91 (m, 2H), |
| | benzoic | carbonyl]- | | | 2.71-2.82 (m, 2H), |
| | acid | 3-{6-[2- | | | 2.91 (dd, 1H), 3.11-3.21 (m, |
| | | (5,6,7,8- | | | 3H), 3.39-3.50 (m, |
| | | tetrahydro- | | | 2H), 4.59 (bs, 1H), |
| | | 1,8- | | | 4.72 (dd, 2H), 6.70 (d, |
| | | naphthyridin- | | | 1H), 6.90 (d, 1H), |
| | | 2- | | | 7.38-7.48 (m, 3H), 7.61 (d, |
| | | yl)ethoxy]pyridin- | | | 1H), 7.82 (d, 1H), |
| | | 3-yl}-L- | | | 8.11 (s, 1H) |
| | | alanine | | | |
| 62.2 | 2- | N-[(2- | 56 | 481 | 1.78-1.86 (m, 2H), |
| | chlorobenzoic | chlorophenyl) | | | 2.52 (t, partially hidden by |
| | acid | carbonyl]-3- | | | DMSOd5, 2H), 2.73 (t, |
| | | {6-[2- | | | 2H), 2.87 (dd, 1H), |
| | | (5,6,7,8- | | | 3.08-3.15 (m, 3H), |
| | | tetrahydro- | | | 3.43 (m partially hidden by |
| | | 1,8- | | | H2O, 2H), 4.53 (ddd, |
| | | naphthyridin- | | | 2H), 4.57 (ddd, 1H), |
| | | 2- | | | 6.67 (d, 1H), 6.74 (d, |
| | | yl)ethoxy]pyridin- | | | 1H), 7.23-7.26 (m, |
| | | 3-yl}-L- | | | 1H), 7.38 9ddd, 1H), |
| | | alanine | | | 7.41-7.47 (m, 2H), |
| | | | | | 7.60 (d, 1H), 7.66 (dd, 1H), |
| | | | | | 7.88 (bs, 1H), 8.05 (dd, |
| | | | | | 1H), 8.78 (d, 1H) |
| 62.3 | 2-chloro- | N-[(2-chloro- | 58 | 498 | 1.77-1.86 (m, 2H), |
| | 4- | 4- | | | 2.73 (t, 2H), 2.87 (dd, 1H), |
| | fluorobenzoic | fluorophenyl) | | | 3.11 (dd, 1H), 3.13 (t, |
| | acid | carbonyl]-3- | | | 2H), 3.42 (bs, 2H), |
| | | {6-[2- | | | 4.53 (dd, 1H), |
| | | (5,6,7,8- | | | 4.57 (ddd, 1H), 6.66 (d, 1H), |
| | | tetrahydro- | | | 6.75 (d, 1H), |
| | | 1,8- | | | 7.26-7.35 (m, 2H), 7.47 (dd, 1H), |
| | | naphthyridin- | | | 7.60 (d, 1H), 7.66 (dd, |
| | | 2- | | | 1H), 8.01 (s, 1H), |
| | | yl)ethoxy]pyridin- | | | 8.04 (d, 1H), 8.82 (d, 1H) |
| | | 3-yl}-L- | | | |
| | | alanine | | | |
| 62.4 | 2-chloro- | N-[(2-chloro- | 9 | 498 | 1.78-1.85 (m, 2H), |
| | 6- | 6- | | | 2.73 (t, 2H), 2.85 (dd, 1H), |
| | fluorobenzoic | fluorophenyl) | | | 3.10 (dd, 1H), 3.13 (t, |
| | acid | carbonyl]-3- | | | 2H), 3.43 (m, 2H), |
| | | {6-[2- | | | 4.54 (ddd, 2H), 4.62 (ddd, |
| | | (5,6,7,8- | | | 1H), 6.66 (d, 1H), |
| | | tetrahydro- | | | 6.74 (d, 1H), 7.26 (dd, 1H), |
| | | 1,8- | | | 7.31 (d, 1H), 7.45 (ddd, |
| | | naphthyridin- | | | 1H), 7.60 (d, 1H), |
| | | 2- | | | 7.66 (dd, 1H), 8.03 (d, 1H), |
| | | yl)ethoxy]pyridin- | | | 8.07 (bs, 1H), 9.11 (d, |
| | | 3-yl}-L- | | | 1H) |
| | | alanine | | | |

The starting material was prepared as follows:

A solution of tert-butyl N-[(benzyloxy)carbonyl]-3-(6-bromopyridin-3-yl)-L-alaninate (3.9 g, 8.96 mmol, described in Example 54), 2-[8-(4-methoxybenzyl)-5,6,7,8-tetrahydro-1,8-naphthyridin-2-yl]ethanol (2.01 g, 6.74 mmol), cesium carbonate (6.57 g, 20.2 mmol) palladium acetate (121 mg, 0.54 mmol) and 2-(di-ter-butylphosphino)-1,1′-binaphthyl (267 mg, 0.67 mmol) in toluene (50 ml) was degassed and heated at 80° C. for 26 hours under argon atmosphere After concentration, a purification by silica gel flash chromatography (20 to 35% ethyl acetate in petroleum ether) afforded tert-butyl N-[(benzyloxy)carbonyl]-3-(6-{2-[8-(4-methoxybenzyl)-5,6,7,8-tetrahydro-1,8-naphthyridin-2-yl]ethoxy}pyridin-3-yl)-L-alaninate (0.76 g, 17%); Mass spectrum [M+H]⁺=653; ¹H NMR Spectrum (DMSOd6) 1.33 (s, 9H), 1.75-1.83 (m, 2H), 2.64 (t, 2H), 2.78 (dd, 1H), 2.91 (dd, 1H), 2.93 (t, 2H), 3.23-3.29 (m, 2H), 3.68 (s, 3H), 4.09 (ddd, 1H), 4.54 (t, 2H), 4.71 (s, 2H), 4.95-5.02 (m, 2H), 6.37 (d, 1H), 6.66 (d, 1H), 6.78 (d, 2H), 7.07 (d, 1H), 7.21 (d, 2H), 7.25-7.36 (m, 5H), 7.52 (dd, 1H), 7.71 (d, 1H), 7.98 (d, 1H).

A mixture of tert-butyl N-[(benzyloxy)carbonyl]-3-(6-{2-[8-(4-methoxybenzyl)-5,6,7,8-tetrahydro-1,8-naphthyridin-2-yl]ethoxy}pyridin-3-yl)-L-alaninate (735 mg, 1.13 mmol) and Pd/C 10% (32 mg) in methanol (15 ml) and DCM (5 ml) was hydrogenated under 1 bar of H₂ for 2 hours, filtered through celite, evaporated and purified by silica gel flash chromatography (0 to 10% THF in DCM) to afford tert-butyl 3-(6-{2-[8-(4-methoxybenzyl)-5,6,7,8-tetrahydro-1,8-naphthyridin-2-yl]ethoxy}pyridin-3-yl)-L-alaninate (536 mg, 92%); Mass spectrum [M+H]⁺=519; ¹H NMR Spectrum (DMSOd6) 1.33 (s, 9H), 1.75-1.83 (m, 2H), 2.64 (t, 2H), 2.78 (dd, 1H), 2.91 (dd, 1H), 2.93 (t, 2H), 3.23-3.29 (m, 2H), 3.68 (s, 3H), 4.09 (ddd, 1H), 4.54 (t, 2H), 4.71 (s, 2H), 4.95-5.02 (m, 2H), 6.37 (d, 1H), 6.66 (d, 1H), 6.78 (d, 2H), 7.07 (d, 1H), 7.21 (d, 2H), 7.25-7.36 (m, 5H), 7.52 (dd, 1H), 7.71 (d, 1H), 7.98 (d, 1H).

The 2-[8-(4-methoxybenzyl)-5,6,7,8-tetrahydro-1,8-naphthyridin-2-yl]ethanol starting material was prepared as follows:

To a solution of 2-[5,6,7,8-tetrahydro-1,8-naphthyridin-2-yl]ethanol (1.877 g, 10 mmol) (WO 2004/058761 page 34) in DMF (2 ml) were added K₂CO₃ (2.76 g, 20 mmol) and 4-methoxybenzyl chloride (1.53 ml, 10.5 mmol). The mixture was heated at 65° C. for 8 hours. After evaporation and extraction with DCM, the residue was purified by silica gel flash chromatography (0 to 15% ethyl acetate in DCM) to afford 2-[8-(4-methoxybenzyl)-5,6,7,8-tetrahydro-1,8-naphthyridin-2-yl]ethanol (2.25 g, 75%); Mass spectrum [M+H]⁺=299; ¹H NMR Spectrum (DMSOd6) 1.75-1.83 (m, 2H), 2.59-2.69 (m, 4H), 3.22-3.29 (m, 2H), 3.67 (ddd, 1H), 3.71 (s, 3H), 4.54 (t, 1H), 4.71 (s 2H), 6.32 (d, 1H), 6.85 (d, 2H), 7.06 (d, 1H), 7.21 (d, 2H).

### Example 63.1

### N-[(2-chlorophenyl)carbonyl]-3-{5-[2-(5,6,7,8-tetrahydro-1,8-naphthyridin-2-yl)ethoxy]pyridin-2-yl}-L-alanine

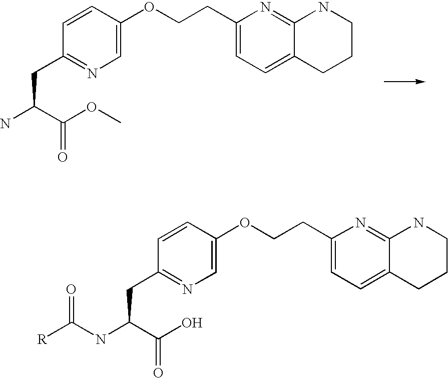

To a solution of methyl 3-{5-[2-(5,6,7,8-tetrahydro-1,8-naphthyridin-2-yl)ethoxy]pyridin-2-yl}-L-alaninate (300 mg, 0.84 mmol, described in Example 14.1) in DMF (1.5 ml) were added HOPO (121 mg, 1.09 mmol), EDCI (209 mg, 1.09 mmol) and 2-chlorobenzoic acid (132 mg, 0.84 mmol); The mixture was stirred at ambient temperature for 18 hours. Water (3 ml) was added. The organic phase was separated and purified by flash chromatography eluting with 40% ethyl acetate in petroleum ether to give methyl N-[(2-chlorophenyl)carbonyl]-3-{5-[2-(5,6,7,8-tetrahydro-1,8-naphthyridin-2-yl)ethoxy]pyridin-2-yl}-L-alaninate as an intermediate which was redissolved in DMF (2 ml). NaOH 6N (0.422 ml, 2.52 mmol) was added and the mixture was stirred at ambient temperature for 3 hours. Purification by C18 reverse phase chromatography (basic conditions) afforded the title compound (238 mg, 60%).

The procedure described above for example 63.1 was repeated by coupling methyl 3-{5-[2-(5,6,7,8-tetrahydro-1,8-naphthyridin-2-yl)ethoxy]pyridin-2-yl}-L-alaninate with the appropriate acid shown in Table 17. Thus were obtained the compounds shown in Table 17:

TABLE 17

| | | | | Mass | |
|---|---|---|---|---|---|
| | | | | Ion | ¹H NMR Data (500 MHz |
| Example | Acid | Name | Yield % | [M + H]⁺ | DMSOd6) |
| 63.1 | 2- | N-[(2- | 60 | 481 | 1.70-1.78 (m, 2H), |
| | chlorobenzoic | chlorophenyl)carbonyl]- | | | 2.61 (t, 2H), 2.88 (t, |
| | acid | 3-{5-[2- | | | 2H), 3.03 (dd, 1H), |
| | | (5,6,7,8- | | | 3.18-3.28 (m, 3H), |
| | | tetrahydro-1,8- | | | 4.27 (t, 2H), |
| | | naphthyridin-2- | | | 4.67 (ddd, 1H), 6.36 (d, |
| | | yl)ethoxy]pyridin- | | | 1H), 6.40 (bs, 1H), |
| | | 2-yl}-L- | | | 7.06 (d, 1H), 7.21 (d, |
| | | alanine | | | 1H), 7.27-7.33 (m, |
| | | | | | 2H), 7.36 (ddd, 1H), |
| | | | | | 7.38-7.45 (m, 2H), |
| | | | | | 8.17 (d, 1H), 8.45 (d, |
| | | | | | 1H) |
| 63.2 | 2-chloro- | N-[(2-chloro-4- | 55 | 499 | 1.71-1.97 (m, 2H), |
| | 4- | fluorophenyl)carbonyl]- | | | 2.62 (t, 2H), 2.89 (t, |
| | fluorobenzoic | 3-{5-[2- | | | 2H), 3.03 (dd, 1H), |
| | acid | (5,6,7,8- | | | 3.20-3.28 (m, 3H), |
| | | tetrahydro-1,8- | | | 4.28 (t, 2H), |
| | | naphthyridin-2- | | | 4.66 (ddd, 1H), 6.37 (d, |
| | | yl)ethoxy]pyridin- | | | 1H), 6.46 (bs, 1H), |
| | | 2-yl}-L- | | | 7.07 (d, 1H), 7.22 (d, |
| | | alanine | | | 1H), 7.25 (ddd, 1H), |
| | | | | | 7.29 (dd, 1H), |
| | | | | | 7.38 (dd, 1H), 7.44 (dd, |
| | | | | | 1H), 8.17 (d, 1H), |
| | | | | | 8.48 (d, 1H) |
| 63.3 | 2-chloro- | N-[(2-chloro-6- | 62 | 499 | 1.70-1.81 (m, 2H), |
| | 6- | fluorophenyl)carbonyl]- | | | 2.62 (t, 2H), 2.90 (t, |
| | fluorobenzoic | 3-{5-[2- | | | 2H), 2.99 (dd, 1H), |
| | acid | (5,6,7,8- | | | 3.21 (dd, 1H), |
| | | tetrahydro-1,8- | | | 3.23-3.27 (m, 2H), 4.28 (t, |
| | | naphthyridin-2- | | | 2H), 4.72 (ddd, 1H), |
| | | yl)ethoxy]pyridin- | | | 6.37 (d, 1H), 6.46 (bs, |
| | | 2-yl}-L- | | | 1H), 7.07 (d, 1H), |
| | | alanine | | | 7.18-7.24 (m, 2H), |
| | | | | | 7.25-7.31 (m, 2H), |
| | | | | | 7.42 (ddd, 1H), |
| | | | | | 8.17 (d, 1H), 8.77 (d, 1H) |
| 63.4 | 2-chloro- | N-[(2-chloro-4- | 66 | 511 | ) 1.69-1.80 (m, 2H), |
| | 4- | methoxyphenyl) | | | 2.61 (t, 2H), 2.88 (t, |
| | methoxybenzoic | carbonyl]-3-{5- | | | 2H), 3.05 (dd, 1H), |
| | acid | [2-(5,6,7,8- | | | 3.20 (dd, 1H), |
| | | tetrahydro-1,8- | | | 3.21-3.26 (m, 2H), 3.78 (s, |
| | | naphthyridin-2- | | | 3H), 4.27 (t, 2H), |
| | | yl)ethoxy]pyridin- | | | 4.66 (ddd, 1H), |
| | | 2-yl}-L- | | | 6.36 (d, 1H), 6.37 (s, 1H), |
| | | alanine | | | 6.93 (dd, 1H), |
| | | | | | 7.00 (d, 1H), 7.05 (d, 1H), |
| | | | | | 7.21 (d, 1H), |
| | | | | | 7.26-7.33 (m, 2H), 8.16 (d, |
| | | | | | 1H), 8.37 (d, 1H) |
| 63.5 | 2-fluoro- | N-[(2-fluoro-4- | 30 | 479 | 1.70-1.78 (m, 2H), |
| | 4- | methylphenyl)carbonyl]- | | | 2.34 (s, 3H), 2.60 (t, |
| | methylbenzoic | 3-{5-[2- | | | 2H), 2.87 (t, 2H), |
| | acid | (5,6,7,8- | | | 3.11 (dd, 1H) |
| | | tetrahydro-1,8- | | | 3.21 (dd, 1H), 3.22 (bs, |
| | | naphthyridin-2- | | | 2H), 4.26 (t, 2H), |
| | | yl)ethoxy]pyridin- | | | 4.50-4.58 (m, 1H), |
| | | 2-yl}-L- | | | 6.33-6.38 (m, 2H), |
| | | alanine | | | 7.05 (d, 1H), |
| | | | | | 7.06-7.12 (m, 2H), 7.17 (d, |
| | | | | | 1H), 7.25 (dd, 1H), |
| | | | | | 7.57 (dd, 1H), |
| | | | | | 8.11 (d, 1H), 8.14-8.20 (m, |
| | | | | | 1H) |
| 63.6 | 2-ethyl-4- | N-[(2-ethyl-4- | 54 | 493 | 0.97 (t, 3H), |
| | fluorobenzoic | fluorophenyl)carbonyl]- | | | 1.69-1.80 (m, 2H), 2.53 (q, |
| | acid | 3-{5-[2- | | | partially hidden by |
| | | (5,6,7,8- | | | DMSOd5, 2H), |
| | | tetrahydro-1,8- | | | 2.61 (t, 2H), 2.88 (t, 2H), |
| | | naphthyridin-2- | | | 3.02 (dd, 1H), |
| | | yl)ethoxy]pyridin- | | | 3.22 (dd, 1H), 3.23 (bs, |
| | | 2-yl}-L- | | | 2H), 4.27 (t, 2H), |
| | | alanine | | | 4.68 (ddd, 1H), |
| | | | | | 6.36 (d, 1H), 6.39 (bs, 1H), |
| | | | | | 6.99-7.08 (m, 3H), |
| | | | | | 7.18-7.23 (m, 2H), |
| | | | | | 7.29 (dd, 1H), |
| | | | | | 8.16 (d, 1H), 8.31 (d, 1H) |

### Example 64.1

### N-[(2-chlorophenyl)carbonyl]-3-(5-{2-[6-(methylamino)pyridin-2-yl]ethoxy}pyridin-2-yl)-L-alanine

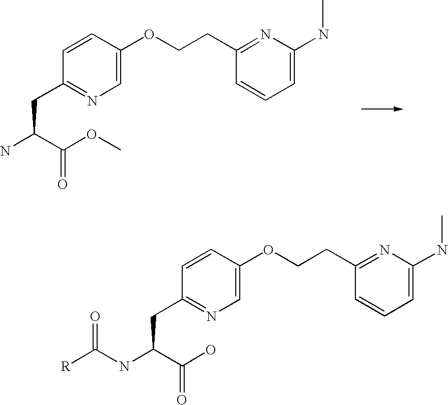

To a solution of methyl 3-(5-{2-[6-(methylamino)pyridin-2-yl]ethoxy}pyridin-2-yl)-L-alaninate (270 mg, 0.82 mmol) in DCM (3 ml) were added HOPO (118 mg, 1.06 mmol), EDCI (203 mg, 1.06 mmol) and 2-chlorobenzoic acid (128 mg, 0.82 mmol). The mixture was stirred at ambient temperature for 24 hours. Saturated NaHCO₃ (3 ml ) was added. The organic phase was separated and purified by flash chromatography eluting with 2% MeOH in DMC to give methyl N-[(2-chlorophenyl)carbonyl]-3-(5-{2-[6-(methylamino)pyridin-2-yl]ethoxy}pyridin-2-yl)-L-alaninate as an intermediate which was redissolved in DMF (2 ml). NaOH 2N (0.55 ml, 3.27 mmol) was added and the mixture was stirred at ambient temperature for 1 hour. Purification by C18 reverse phase chromatography (basic conditions) afforded the title compound (230 mg, 62%).

The procedure described above for example 64.1 was repeated by coupling methyl 3-(5-{2-[6-(methylamino)pyridin-2-yl]ethoxy}pyridin-2-yl)-L-alaninate with the acid shown in Table 18. Thus were obtained the compounds shown in Table 18:

TABLE 18

| | | | | Mass | ¹H NMR Data (500 MHz |
|---|---|---|---|---|---|
| Example | Acid | Name | Yield % | Ion | DMSOd6) |
| 64.1 | 2- | N-[(2- | 62 | 455 | 2.74 (d, 3H), 2.96 (t, 2H), |
| | chlorobenzoic | chlorophenyl) | | | 3.04 (dd, 1H), 3.21 (dd, |
| | acid | carbonyl]- | | | 1H), 4.34 (t, 2H), |
| | | 3-(5-{2-[6- | | | 4.61-4.70 (m, 1H), 6.27 (d, |
| | | (methylamino) | | | 1H), 6.36 (q, 1H), 6.43 (d, |
| | | pyridin- | | | 1H), 7.21 (d, 1H), |
| | | 2- | | | 7.26-7.34 (m, 3H), 7.36 (ddd, |
| | | yl]ethoxy}pyridin- | | | 1H), 7.38-7.46 (m, 2H), |
| | | 2-yl)- | | | 8.18 (d, 1H), 8.45 (bs, 1H) |
| | | L-alanine | | | |
| 64.2 | 2-chloro- | N-[(2- | 51 | 473 | 2.73 (d, 3H), 2.93 (t, 2H), |
| | 4- | chloro-4- | | | 3.03 (dd, 1H), 3.21 (dd, |
| | fluorobenzoic | fluorophenyl) | | | 1H), 4.34 (t, 2H), |
| | acid | carbonyl]- | | | 4.69 (ddd, 1H), 6.27 (d, 1H), |
| | | 3-(5-{2-[6- | | | 6.36 (q, 1H), 6.43 (d, 1H), |
| | | (methylamino) | | | 7.21 (d, 1H), 7.26 (ddd, |
| | | pyridin- | | | 1H), 6.28-6.38 (m, 3H), |
| | | 2- | | | 7.45 (dd, 1H), 8.19 (d, |
| | | yl]ethoxy}pyridin- | | | 1H), 8.60 (d, 1H) |
| | | 2-yl)- | | | |
| | | L-alanine | | | |
| 64.3 | 2-chloro- | N-[(2- | 55 | 473 | 2.74 (d, 3H), 2.96 (t, 2H), |
| | 6- | chloro-6- | | | 2.99 (dd, 1H), 3.20 (dd, |
| | fluorobenzoic | fluorophenyl) | | | 1H), 4.34 (t, 2H), |
| | acid | carbonyl]- | | | 4.61-4.69 (m, 1H), 6.27 (d, |
| | | 3-(5-{2-[6- | | | 1H), 6.36 (q, 1H), 6.43 (d, |
| | | (methylamino) | | | 1H), 7.17-7.25 (m, 2H), |
| | | pyridin- | | | 7.26-7.33 (3H), 7.41 (ddd, |
| | | 2- | | | 1H), 8.16 (d, 1H), |
| | | yl]ethoxy}pyridin- | | | 8.67 (bs, 1H) |
| | | 2-yl)- | | | |
| | | L-alanine | | | |
| 64.4 | 2-chloro- | N-[(2- | 55 | 485 | 2.74 (d, 3H), 2.96 (t, 2H), |
| | 4- | chloro-4- | | | 3.06 (dd, 1H), 3.19 (dd, |
| | methoxybenzoic | methoxyphenyl) | | | 1H), 3.78 (s, 3H), 4.34 (t, |
| | acid | carbonyl]- | | | 2H), 4.70 (ddd, 1H), |
| | | 3-(5-{2- | | | 6.27 (d, 1H), 6.36 (q, 1H), |
| | | [6- | | | 6.43 (d, 1H), 6.94 (dd, 1H), |
| | | (methylamino) | | | 7.01 (d, 1H), 7.22 (d, 1H), |
| | | pyridin- | | | 7.25-7.35 (m, 3H), |
| | | 2- | | | 8.19 (d, 1H), 8.48 (d, 1H) |
| | | yl]ethoxy}pyridin- | | | |
| | | 2-yl)- | | | |
| | | L-alanine | | | |
| 64.5 | 2-ethyl-4- | N-[(2-ethyl- | 46 | 467 | 0.96 (t, 3H), 2.52 (q |
| | fluorobenzoic | 4- | | | partially hidden by |
| | acid | fluorophenyl) | | | DMSOd5, 2H), 2.74 (d, |
| | | carbonyl]- | | | 3H), 2.96 (t, 2H), |
| | | 3-(5-{2-[6- | | | 3.03 (dd, 1H), 3.21 (dd, 1H), |
| | | (methylamino) | | | 4.35 (t, 2H), 4.70-4.78 (m, |
| | | pyridin- | | | 1H), 6.27 (d, 1H), 6.36 (q, |
| | | 2- | | | 1H), 6.43 (d, 1H), |
| | | yl]ethoxy}pyridin- | | | 6.99-7.09 (m, 2H), 7.18 (ddd, |
| | | 2-yl)- | | | 1H), 7.22 (d, 1H), |
| | | L-alanine | | | 7.27-7.36 (m, 2H), 8.19 (d, |
| | | | | | 1H), 8.47 (d, 1H) |
| 64.6 | 2,6- | N-[(2,6- | 35 | 489 | 2.74 (d, 3H), 2.96 (t, 2H), |
| | dichlorobenzoic | dichlorophenyl) | | | 3.01 (dd, 1H), 3.19 (dd, |
| | acid | carbonyl]- | | | 1H), 4.34 (t, 2H), |
| | | 3-(5-{2- | | | 4.70-4.79 (m, 1H), 6.27 (d, |
| | | [6- | | | 1H), 6.37 (q, 1H), 6.43 (d, |
| | | (methylamino) | | | 1H), 7.21 (d, 1H), |
| | | pyridin- | | | 7.24-7.33 (m, 2H), 7.37 (dd, |
| | | 2- | | | 1H), 7.39-7.44 (m, 2H), |
| | | yl]ethoxy}pyridin- | | | 8.17 (d, 1H), 8.75 (bs, 1H) |
| | | 2-yl)- | | | |
| | | L-alanine | | | |

The starting material was prepared as follows:

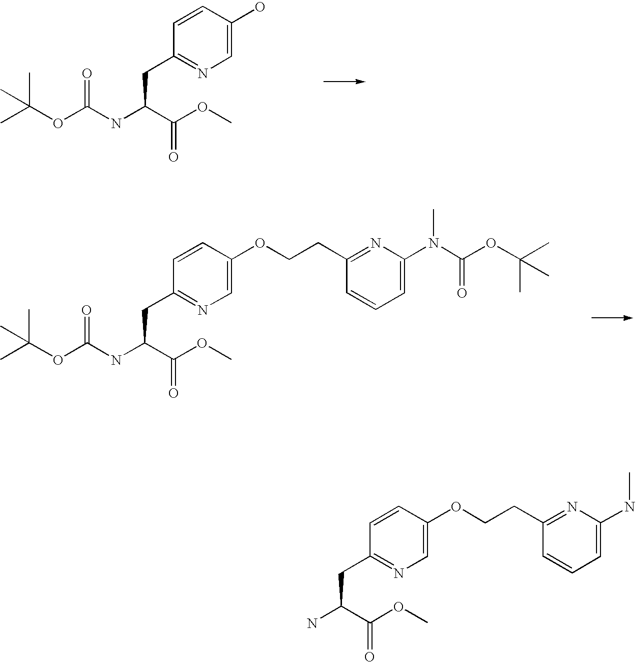

Triphenylphosphine (3.59 g, 13.7 mmol), tert-Butyl [6-(2-hydroxyethyl)pyridin-2-yl]methylcarbamate (Journal of Medicinal Chemistry 2000, 43(1), 22-26), (3.81 g, 13.7 mmol) and DTAD (3.15 g, 13.7 mmol) were added to a suspension of methyl N-(tert-butoxycarbonyl)-3-(5-hydroxypyridin-2-yl)-L-alaninate (2.7 g, 9.11 mmol) (WO98/58902, page 181) in DCM (60 ml) at −5° C., under an argon atmosphere. The mixture was stirred at −5° C. for one hour and at room temperature for 3 hours. After evaporation, the residue was purified by silica gel flash chromatography eluting with 20 to 30% ethyl acetate in petroleum ether to give methyl N-(tert-butoxycarbonyl)-3-[5-(2-{6-[(tert-butoxycarbonyl)(methyl)amino]pyridin-2-yl}ethoxy)pyridin-2-yl]-L-alaninate as an oil (4.54 g, 94%); ¹H NMR Spectrum: (DMSO-d6) 1.32 (s, 9H), 1.45 (s, 9H), 2.95 (dd, 1H), 3.02 (dd, 1H), 3.14 (t, 2H), 3.26 (s, 3H), 3.32 (s, 3H), 4.36 (dd, 1H), 4.39 (t, 2H), 7.09 (d, 1H), 7.17 (d, 1H), 7.22 (d, 1H), 7.31 (dd, 1H), 7.45 (d, 1H), 7.68 (dd, 1H), 8.18 (d, 1H).

TFA (10 ml) was added to a solution of methyl N-(tert-butoxycarbonyl)-3-[5-(2-{6-[(tert-butoxycarbonyl)(methyl)amino]pyridin-2-yl}ethoxy)pyridin-2-yl]-L-alaninate (4.54 g, 8.57 mmol) in DCM (10 ml). The mixture was stirred at room temperature overnight. After evaporation, the residue was dissolved in NH₃/MeOH 7N and evaporated. The residue was purified by silica gel flash chromatography eluting with 2% MeOH in DCM to afford methyl 3-(5-{2-[6-(methylamino)pyridin-2-yl]ethoxy}pyridin-2-yl)-L-alaninate as an oil (2.1 g, 74%); Mass spectrum [M+H]⁺=331; ¹H NMR Spectrum 1.84 (bs, 2H), 2.74 (d, 3H), 2.83 (dd, 1H), 1.93-2.97 (m, 3H), 3.57 (s, 3H), 3.69 (dd, 1H), 4.34 (t, 2H), 6.26 (d, 1H), 6.36 (q, 1H), 6.43 (d, 1H), 7.15 (d, 1H), 7.28-7.32 (m, 2H), 8.15 (d, 1H).

### Example 65

### N-[(2,6-dichlorophenyl)carbonyl]-3-{5-[3-(5,6,7,8-tetrahydro-1,8-naphthyridin-2-yl)propyl]pyridin-2-yl}-L-alanine

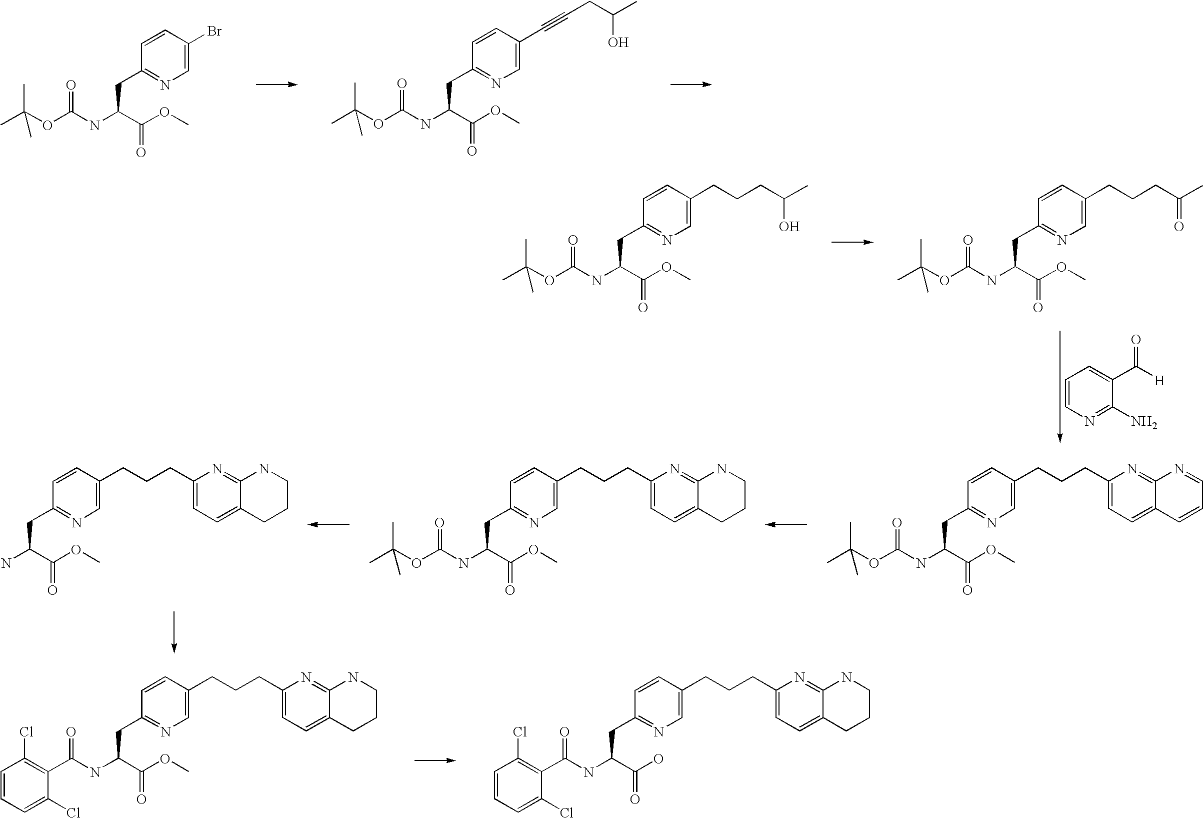

Sodium hydroxide 6N (0.092 μl, 0.55 μmol) was added to a stirred solution of methyl N-[(2,6-dichlorophenyl)carbonyl]-3-{5-[3-(5,6,7,8-tetrahydro-1,8-naphthyridin-2-yl)propyl]pyridin-2-yl}-L-alaninate (97 mg, 0.18 mmol), in THF (2 ml) at 25° C. The resulting solution was stirred at 25° C. for 3 hours. The mixture is concentrated and dissolved in DMF-Water. The reaction mixture was purified by C18 reverse phase chromatography (basic conditions) to afford the title compound (75 mg, 80%) as a white solid; ¹H NMR Spectrum (DMSOd6+TFAd) 1.80-1.91 (m, 2H), 1.93-2.07 (m, 2H), 2.68-2.79 (m, 4H), 2.83 (t, 2H), 3.37 (dd, 1H), 3.41-3.47 (m, 2H), 3.67 (dd, 1H), 5.14 (dd, 1H), 6.64 (d, 1H), 7.38-7.50 (m, 3H), 7.63 (d, 1H), 8.02 (d, 1H), 8.50 (dd, 1H), 8.85 (d, 1H).

The starting material was prepared as follows:

Bis(triphenylphosphine) palladium (II) chloride (1.172 g, 1.67 mmol), CuI (212 mg, 1.11 mmol), 4-pentyn-2-ol (5.23 ml, 55.63 mmol) were added under nitrogen to a solution of methyl 3-(5-bromopyridin-2-yl)-N-(tert-butoxycarbonyl)-L-alaninate (10 g, 27.84 mmol, described in example 42.1), and TEA (15.52 ml, 111.35 mmol) in DMF (50 ml). The reaction was stirred at 80° C. under nitrogen for 2 hours. The mixture was extracted with ethyl acetate and purified by silica gel flash chromatography (40 to 70% ethyl acetate in petroleum ether) to afford methyl N-(tert-butoxycarbonyl)-3-[5-(4-hydroxypent-1-yn-1-yl)pyridin-2-yl]-L-alaninate (8.6 g, 85%); Mass spectrum [M+H]⁺=363; ¹H NMR Spectrum (CDCl3) 1.33 (d, 3H), 1.42 (s, 9H), 2.02 (bs, 1H), 2.58 (dd, 1H), 2.64 (dd, 1H), 3.25 (dd, 1H), 3.31 (dd, 1H), 3.69 (s, 3H), 4.02-4.10 (m, 1H), 4.64-4.71 (m, 1H), 5.75 (d, 1H), 7.08 (d, 1H), 7.61 (dd, 1H), 8.51 (d, 1H).

A mixture of methyl N-(tert-butoxycarbonyl)-3-[5-(4-hydroxypent-1-yn-1-yl)pyridin-2-yl]-L-alaninate (7.82 g, 21.58 mmol) and Pd/C 10% (800 mg) in methanol (270 ml) was hydrogenated under 4 bar of H₂ for 15 hours, filtered through celite, evaporated and purified by silica gel flash chromatography (80 to 100% ethyl acetate in petroleum ether) to afford methyl N-(tert-butoxycarbonyl)-3-[5-(4-hydroxypentyl)pyridin-2-yl]-L-alaninate (7.21 g, 91%); Mass spectrum [M+H]⁺=367; ¹H NMR Spectrum (CDCl3) 1.19 (d, 3H), 1.42 (s, 9H), 1.43-1.52 (m, 2H), 1.59-1.69 (m, 1H), 1.69-1.80 (m, 1H), 1.91 (bs, 1H), 2.55-2.66 (m, 2H), 3.23 (dd, 1H), 3.29 (dd, 1H), 3.69 (s, 3H), 3.78-3.87 (m, 1H), 4.62-4.71 (m, 1H), 5.89 (d, 1H), 7.07 (d, 1H), 7.44 (dd, 1H), 8.34 (d, 1H).

A solution of methyl N-(tert-butoxycarbonyl)-3-[5-(4-hydroxypentyl)pyridin-2-yl]-L-alaninate (7.21 g, 19.67 mmol) in DMC (150 ml) and pyridinium dichromate (7.41 g, 19.7 mmol) was stirred at ambient temperature for 5 hours. 3 further equivalents of pyridinium dichromate (22.23 g) were added and the mixture was stirred overnight. After filtration of the insoluble and extraction with DMC, the residue was purified by silica gel flash chromatography (60 to 75% ethyl acetate in petroleum ether) to afford methyl N-(tert-butoxycarbonyl)-3-[5-(4-oxopentyl)pyridin-2-yl]-L-alaninate (6.1 g, 85%); Mass spectrum [M+H]⁺=365; ¹H NMR Spectrum (CDCl3) 1.42 (s, 9H), 1.84-1.93 (m, 2H), 2.13 (s, 3H), 2.46 (t, 2H), 2.58 (t, 2H), 3.22 (dd, 1H), 3.29 (dd, 1H), 3.70 (s, 3H), 4.62-4.72 (m, 1H), 5.88 (d, 1H), 7.07 (d, 1H), 7.44 (dd, 1H), 8.33 (d, 1H).

To a solution of methyl N-(tert-butoxycarbonyl)-3-[5-(4-oxopentyl)pyridin-2-yl]-L-alaninate (5.72 g, 15.7 mmol) in ethanol (135 ml), were added 2-aminopyridine-3-carboxaldehyde (2.11 g, 17.3 mmol) and L-proline (0.994 g, 8.63 mmol). The mixture was stirred overnight under argon atmosphere. After evaporation, the residue was taken up in ethyl acetate, washed with sat NaHCO₃ and purified by silica gel flash chromatography (1 to 8% methanol in ethyl acetate) to afford methyl N-(tert-butoxycarbonyl)-3-{5-[3-(1,8-naphthyridin-2-yl)propyl]pyridin-2-yl}-L-alaninate (3.23 g, 46%); Mass spectrum [M+H]⁺=451; ¹H NMR Spectrum (CDCl3) 1.42 (s, 9H), 2.20-2.30 (m, 2H), 2.72 (t, 2H), 3.09 (t, 2H), 3.22 (dd, 1H), 3.29 (dd, 1H), 3.69 (s, 3H), 4.62-4.71 (m, 1H), 5.89 (d, 1H), 7.06 (d, 1H), 7.37 (d, 1H), 7.46 (dd, 1H), 7.49 (dd, 1H), 8.11 (d, 1H), 8.17 (dd, 1H), 8.36 (d, 1H), 9.09 (dd, 1H).

A mixture of methyl N-(tert-butoxycarbonyl)-3-{5-[3-(1,8-naphthyridin-2-yl)propyl]pyridin-2-yl}-L-alaninate (330 mg, 0.73 mmol) and Pd/C 10% (50 mg) in ethanol (20 ml) was hydrogenated under 4 bar of H₂ for 4 hours, filtered through celite, evaporated and purified by silica gel flash chromatography (5 to 8% methanol in DMC) to afford methyl N-(tert-butoxycarbonyl)-3-{5-[3-(5,6,7,8-tetrahydro-1,8-naphthyridin-2-yl)propyl]pyridin-2-yl}-L-alaninate (251 mg, 75%); Mass spectrum [M+H]⁺=455; ¹H NMR Spectrum (CDCl3) 1.42 (s, 9H), 1.87-2.02 (m, 4H), 2.55-2.65 (m, 4H), 2.69 (t, 2H), 3.20 (dd, 1H), 3.27 (dd, 1H), 3.37-3.44 (m, 2H), 3.68 (s, 3H), 4.62-4.61 (m, 1H), 4.95 (bs, 1H), 5.90 (d, 1H), 6.33 (d, 1H), 7.03 (d, 1H), 7.07 (d, 1H), 7.43 (dd, 1H), 8.34 (s, 1H).

TFA (1 ml) was added to a solution of methyl N-(tert-butoxycarbonyl)-3-{5-[3-(5,6,7,8-tetrahydro-1,8-naphthyridin-2-yl)propyl]pyridin-2-yl}-L-alaninate (250 mg, 0.55 mmol) in DMC (1 ml ). The reaction was stirred for 2 hours. The reaction mixture was then concentrated to dryness, taken-up in DMC. An excess of a solution of NH₃/MeOH 7N was added and the mixture was concentrated to dryness. The residue was purified by flash chromatography on silica gel eluting with 5 to 20% methanol in DMC to afford methyl 3-{5-[3-(5,6,7,8-tetrahydro-1,8-naphthyridin-2-yl)propyl]pyridin-2-yl}-L-alaninate (186 mg, 95%) as a clear white foam; Mass spectrum [M+H]⁺=355; ¹H NMR Spectrum (CDCl3) 1.88-1.96 (m, 2H), 1.96-2.06 (m, 2H), 2.66 (t, 2H), 2.70-2.77 (m, 4H), 3.04 (dd, 1H), 3.25 (dd, 1H), 3.45-3.52 (m, 2H), 3.73 (s, 3H), 3.98 (dd, 1H), 6.31 (d, 1H), 7.11 (d, 1H), 7.28 (d, 1H), 7.51 (dd, 1H), 8.36 (s, 1H), 8.76 (bs, 1H).

Dichlorobenzoyl chloride (108 mg, 0.51 mmol) in solution in DMC (1.5 ml) was added portionwise under nitrogen to a stirred solution of (methyl 3-{5-[3-(5,6,7,8-tetrahydro-1,8-naphthyridin-2-yl)propyl]pyridin-2-yl}-L-alaninate (182 mg, 0.51 mmol) and triethylamine (0.143 ml, 1.03 mmol) in DMC (3.5 ml ) over a period of 15 minutes at 0° C. The resulting solution was stirred at 25° C. for 2 hours. The reaction mixture was extracted with DMC (40 ml) and purified by C18 reverse phase chromatography (basic conditions) to afford methyl N-[(2,6-dichlorophenyl)carbonyl]-3-{5-[3-(5,6,7,8-tetrahydro-1,8-naphthyridin-2-yl)propyl]pyridin-2-yl}-L-alaninate (99 mg, 37%) as a clear white gum; Mass spectrum [M+H]⁺=527; ¹H NMR Spectrum (CDCl3) 1.87-2.0 (m, 4H), 2.53-2.64 (m, 4H), 2.69 (t, 2H), 3.46-3.49 (m, 4H), 3.73 (s, 3H), 4.99 (bs, 1H), 5.20 (ddd, 1H), 6.32 (d, 1H), 7.07 (d, 1H), 7.11 (d, 1H), 7.23 (dd, 1H), 7.27-7.30 (m, 2H), 7.41 (d, 1H), 7.44 (dd, 1H), 8.26 (d, 1H).

### Example 66.1

### N-[(2-chlorophenyl)carbonyl]-3-{5-[3-(5,6,7,8-tetrahydro-1,8-naphthyridin-2-yl)propyl]thiophen-2-yl}-L-alanine

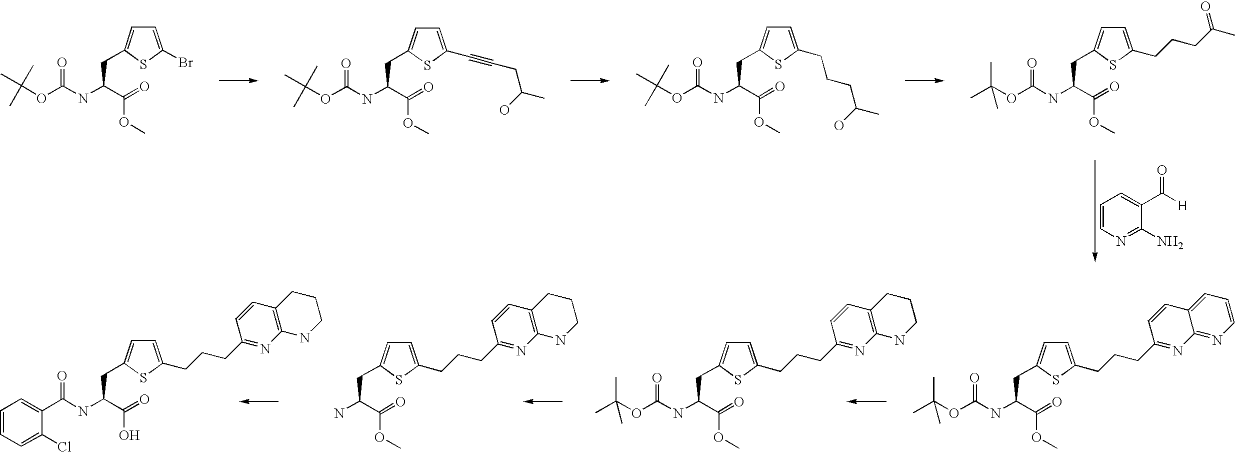

o-Benzotriazol-1-yl-N,N,N′,N′-tetramethyluronium tetrafluoroborate (268 mg, 0.83 mmol), 4-methylmorpholine (0.084 ml, 0.76 mmol) and 2-chlorobenzoic acid (109 mg, 0.70 mmol), were added at 25° C. under argon atmosphere to a stirred solution of methyl 3-{5-[3-(5,6,7,8-tetrahydro-1,8-naphthyridin-2-yl)propyl]thiophen-2-yl}-L-alaninate (250 mg, 0.70 mmol) in DMF (2.5 ml). The resulting solution was stirred at 25° C. for 2 hours. Sodium hydroxide (0.464 ml, 2.78 mmol) was added. The resulting solution was stirred at 25° C. for 1 hour. After evaporation, the residue was purified by C18 reverse phase chromatography (basic conditions) to afford the title compound (187 mg, 55.6%) as a beige solid.

The procedure described above for example 66.1 was repeated by coupling methyl 3-{5-[3-(5,6,7,8-tetrahydro-1,8-naphthyridin-2-yl)propyl]thiophen-2-yl}-L-alaninate with the acid shown in Table 19. Thus were obtained the compounds shown in Table 19:

TABLE 19

| | | | | Mass | ¹H NMR Data (500 MHz |
|---|---|---|---|---|---|
| Example | Acid | Name | Yield % | Ion | DMSOd6) |
| 66.1 | 2- | N-[(2- | 55 | 484 | 1.70-1.78 (m, 2H), |
| | chlorobenzoic | chlorophenyl)carbonyl]- | | | 1.82-1.92 (m, 2H), 2.46 (t, |
| | acid | 3-{5- | | | 2H), 2.60 (t, 2H), 2.70 (t, |
| | | [3-(5,6,7,8- | | | 2H), 3.15 (dd, 1H), |
| | | tetrahydro-1,8- | | | 3.20-3.26 (m, 2H), 3.33 (dd, |
| | | naphthyridin- | | | 1H), 4.36 (bs, 1H), |
| | | 2- | | | 6.24 (d, 1H), 6.40 (bs, 1H), |
| | | yl)propyl]thiophen- | | | 6.61 (d, 1H), 6.68 (d, |
| | | 2-yl}-L- | | | 1H), 7.03 (d, 1H), |
| | | alanine | | | 7.39-7.39 (m, 2H), 7.43 (ddd, |
| | | | | | 1H), 7.46 (d, 1H), |
| | | | | | 8.29 (bs, 1H) |
| 66.2 | 2-chloro- | N-[(2-chloro- | 52 | 502 | 1.71-1.78 (m, 2H), |
| | 4- | 4- | | | 1.82-1.91 (m, 2H), 2.46 (t, |
| | fluorobenzoic | fluorophenyl)carbonyl]- | | | 2H), 2.60 (t, 2H), 2.69 (t, |
| | acid | 3-{5- | | | 2H), 3.14 (dd, 1H), |
| | | [3-(5,6,7,8- | | | 3.20-3.26 (m, 2H), 3.33 (dd, |
| | | tetrahydro-1,8- | | | 1H), 4.35 (bs, 1H), |
| | | naphthyridin- | | | 6.24 (d, 1H), 6.38 (bs, 1H), |
| | | 2- | | | 6.61 (d, 1H), 6.68 (s, |
| | | yl)propyl]thiophen- | | | 1H), 7.02 (d, 1H), |
| | | 2-yl}-L- | | | 7.25 (dd, 1H), 7.42 (dd, 1H), |
| | | alanine | | | 7.46 (dd, 1H), 8.29 (bs, |
| | | | | | 1H) |
| 66.3 | 2-chloro- | N-[(2-chloro- | 56 | 502 | 1.70-1.79 (m, 2H), |
| | 6- | 6- | | | 1.82-1.92 (m, 2H), 2.47 (t, |
| | fluorobenzoic | fluorophenyl)carbonyl]- | | | 2H), 2.60 (t, 2H), 2.70 (t, |
| | acid | 3-{5- | | | 2H), 3.11 (dd, 1H), |
| | | [3-(5,6,7,8- | | | 3.19-3.30 (m, 3H), 4.52 (ddd, |
| | | tetrahydro-1,8- | | | 1H), 6.26 (d, 1H), |
| | | naphthyridin- | | | 6.49 (bs, 1H), 6.62 (d, 1H), |
| | | 2- | | | 6.68 (s, 1H), 7.07 (d, |
| | | yl)propyl]thiophen- | | | 1H), 7.25 (dd, 1H), |
| | | 2-yl}-L- | | | 7.32 (d, 1H), 7.44 (ddd, 1H), |
| | | alanine | | | 8.91 (d, 1H) |
| 66.4 | 2,6- | N-[(2,6- | 58 | 518 | 1.71-1.78 (m, 2H), |
| | dichlorobenzoic | dichlorophenyl) | | | 1.81-1.91 (m, 2H), 2.46 (t, |
| | acid | carbonyl]-3- | | | 2H), 2.60 (t, 2H), 2.70 (t, |
| | | {5-[3-(5,6,7,8- | | | 2H), 3.12 (dd, 1H), |
| | | tetrahydro-1,8- | | | 3.20-3.28 (m, 3H), |
| | | naphthyridin- | | | 4.48-4.58 (m, 1H), 6.25 (d, 1H), |
| | | 2- | | | 6.45 (bs, 1H), 6.61 (d, |
| | | yl)propyl]thiophen- | | | 1H), 6.72 (s, 1H), |
| | | 2-yl}-L- | | | 7.04 (d, 1H), 7.39 (dd, 1H), |
| | | alanine | | | 7.42-7.46 (m, 2H), |
| | | | | | 8.82 (bs, 1H) |

The starting material was prepared as follows:

A suspension of methyl 3-(5-bromo-2-thienyl)-N-(tert-butoxycarbonyl)-L-alaninate (7.25 g, 19.9 mmol, described in Example 29), bis(triphenylphosphine) palladium (II) chloride (835 mg, 1.19 mmol), CuI (152 mg, 0.8 mmol), 4-pentyn-2-ol (3.74 ml, 39.8 mmol) and TEA (11.09 ml, 79.6 mmol) in DMF (60 ml) was stirred at 80° C. under nitrogen for 1 h30. The mixture was extracted with ethyl acetate and purified by silica gel flash chromatography (20 to 80% ethyl acetate in petroleum ether) to afford methyl N-(tert-butoxycarbonyl)-3-[5-(4-hydroxypent-1-yn-1-yl)thiophen-2-yl]-L-alaninate (7 g, 96%); Mass spectrum [M+H]⁺=368; ¹H NMR Spectrum (DMSOd6) 1.15 (d, 3H), 1.36 (s, 9H), 2.44 (dd, 1H), 2.53 (dd, partially hidden by DMSOd5, 1H), 3.04 (dd, 1H), 3.20 (dd, 1H), 3.63 (s, 3H), 3.77-3.85 (m, 1H), 4.15 (ddd, 1H), 4.85 (d, 1H), 6.81 (d, 1H), 7.02 (d, 1H), 7.39 (d, 1H).

A mixture of methyl N-(tert-butoxycarbonyl)-3-[5-(4-hydroxypent-1-yn-1-yl)thiophen-2-yl]-L-alaninate (11 g, 30.2 mmol) and Pd/C 10% (700 mg) in methanol (100 ml) was hydrogenated under 4 bar of H₂ for 3 hours. Pd/C 10% (700 mg) was added to and the mixture was hydrogenated further for 4 hours then filtered through celite, evaporated and purified by silica gel flash chromatography (20 to 80% ethyl acetate in petroleum ether) to afford methyl N-(tert-butoxycarbonyl)-3-[5-(4-hydroxypentyl)thiophen-2-yl]-L-alaninate (8.9 g, 80%); ¹H NMR Spectrum (DMSO-d6) 1.03 (d, 3H), 1.27-1.43 (m, 11H), 1.50-1.71 (m, 2H), 2.63-2.77 (m, 2H), 3.02 (dd, 1H), 3.15 (dd, 1H), 3.59 (ddd, 1H), 3.63 (s, 3H), 4.09-4.19 (m, 1H), 4.37 (d, 1H), 6.63 (d, 1H), 6.69 (d, 1H), 7.32 (d, 1H).

A solution of methyl N-(tert-butoxycarbonyl)-3-[5-(4-hydroxypentyl)thiophen-2-yl]-L-alaninate (8.37 g, 22.53 mmol) and pyridinium dichromate (25.43 g, 67.6 mmol) in DMC (250 ml) was stirred at ambient temperature for 24 hours. After filtration of the insoluble and extraction with DMC, the residue was purified by silica gel flash chromatography (15 to 30% ethyl acetate in petroleum ether) to afford methyl N-(tert-butoxycarbonyl)-3-[5-(4-oxopentyl)thiophen-2-yl]-L-alaninate (5.65 g, 68%); ¹H NMR Spectrum (DMSOd6) 1.37 (s, 9H), 1.70-1.80 (m, 2H), 2.07 (s, 3H), 2.47 (t, 2H), 2.68 (t, 2H), 3.02 (dd, 1H), 3.15 (dd, 1H), 3.63 (s, 3H), 4.14 (ddd, 1H), 6.63 (d, 1H), 6.70 (d, 1H), 7.33 (d, 1H).

To a solution of methyl N-(tert-butoxycarbonyl)-3-[5-(4-oxopentyl)thiophen-2-yl]-L-alaninate (2.05 g, 16.79 mmol) in ethanol (130 ml) were added 2-aminopyridine-3-carboxaldehyde (5.58 g, 15.1 mmol) and L-proline (0.966 g, 8.39 mmol). The mixture was stirred overnight under argon atmosphere. After evaporation, the residue was taken up in DMC, washed with sat NaHCO₃ and purified by silica gel flash chromatography (2 to 5% methanol in DMC) to afford methyl N-(tert-butoxycarbonyl)-3-{5-[3-(1,8-naphthyridin-2-yl)propyl]thiophen-2-yl}-L-alaninate (4.32 g, 56%); Mass spectrum [M+H]⁺=456; ¹H NMR Spectrum (DMSOd6) 1.34 (s, 9H), 2.07-2.17 (m, 2H), 2.83 (t, 2H), 2.98-3.08 (m, 3H), 3.16 (dd, 1H), 3.63 (s, 3H), 4.14 (ddd, 1H), 6.67-6.74 (m, 2H), 7.33 (d, 1H), 7.55 (d, 1H), 7.58 (dd, 1H), 8.37 (d, 1H), 8.42 (dd, 1H), 9.03 (dd, 1H).

A suspension of N-(tert-butoxycarbonyl)-3-{5-[3-(1,8-naphthyridin-2-yl)propyl]thiophen-2-yl}-L-alaninate (3.8 g, 8.34 mmol) and Pd/C 10% (0.335 g) in EtOH (200 ml) was hydrogenated under 4 atmosphere for 3 hours. The resulting solution was filtered and the filtrate was concentrated to dryness to afford the crude methyl N-(tert-butoxycarbonyl)-3-{5-[3-(5,6,7,8-tetrahydro-1,8-naphthyridin-2-yl)propyl]thiophen-2-yl}-L-alaninate (3.00 g, 78%) as a pale colorless liquid. Mass spectrum [M+H]⁺=460; ¹H NMR Spectrum (DMSOd6) 1.36 (s, 9H), 1.72-1.80 (m, 2H), 1.83-1.93 (m, 2H), 2.46 (t, 2H), 2.60 (t, 2H), 2.70 (t, 2H), 3.01 (dd, 1H), 3.14 (dd, 1H), 3.19-3.27 (m, 2H), 3.62 (s, 3H), 4.13 (ddd, 1H), 6.25 (d, 1H), 6.29 (bs, 1H), 6.63 (d, 1H), 6.68 (d, 1H), 7.03 (d, 1H), 7.32 (d, 1H).

Trifluoroacetic acid (10.06 ml, 130.55 mmol) was added under argon atmosphere to a stirred solution of N-(tert-butoxycarbonyl)-3-{5-[3-(5,6,7,8-tetrahydro-1,8-naphthyridin-2-yl)propyl]thiophen-2-yl}-L-alaninate (3 g, 6.53 mmol) in DMC (20 ml). The resulting solution was stirred at 25° C. for 3 hours. The reaction mixture was then concentrated to dryness, taken-up in DMC. An excess of a solution of NH₃/MeOH 7N was added. After evaporation to dryness, the residue was purified by flash chromatography on silica gel eluting with 5 to 10% methanol in DMC to afford methyl 3-{5-[3-(5,6,7,8-tetrahydro-1,8-naphthyridin-2-yl)propyl]thiophen-2-yl}-L-alaninate (2.400 g,) as a pale yellow gum; Mass spectrum [M+H]⁺=360; ¹H NMR Spectrum (DMSOd6) 1.70-1.79 (m, 2H), 1.83-1.92 (m, 2H), 1.97 (bs, 2H), 2.46 (t, 2H), 2.60 (t, 2H), 2.70 (t, 2H), 3.20-3.26 (m, 2H), 3.53 (t, 1H), 3.60 (s, 3H), 6.24 (d, 1H), 6.27 (bs, 1H), 6.63 (s, 2H), 7.02 (d, 1H).

### Example 67.1

### N-[(2-chloro-6-fluorophenyl)carbonyl]-4-[3-(5,6,7,8-tetrahydro-1,8-naphthyridin-2-yl)propyl]-L-phenylalanine

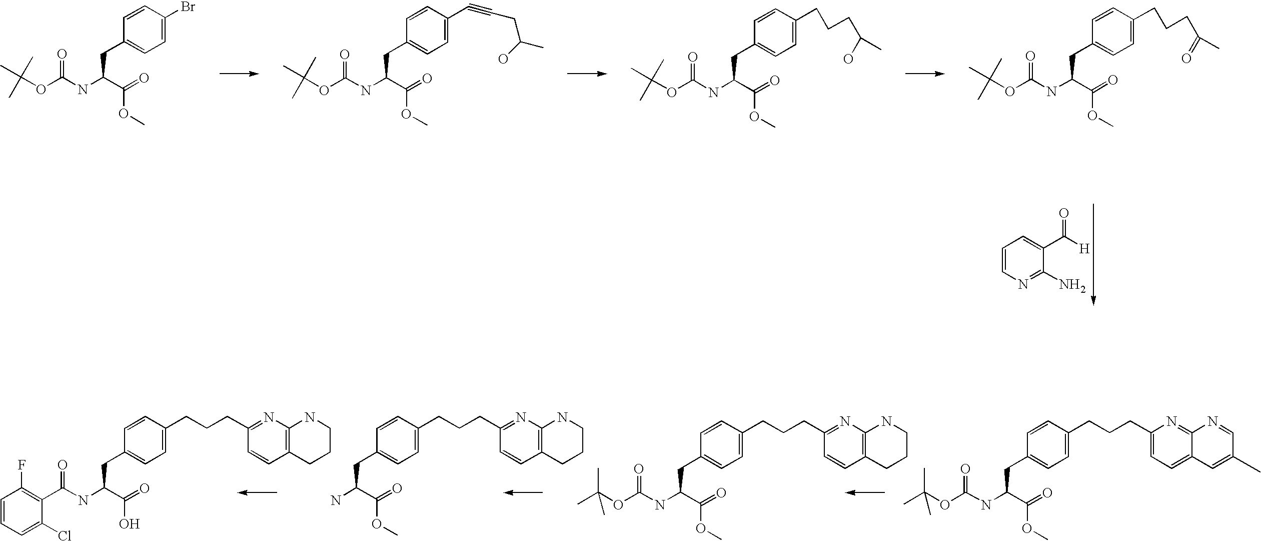

To a solution of methyl 4-[3-(5,6,7,8-tetrahydro-1,8-naphthyridin-2-yl)propyl]-L-phenylalaninate (300 mg, 0.85 mmol) in DMF (1.5 ml) were added HOPO (122 mg, 1.1 mmol), EDCI (211 mg, 1.1 mmol) and 2-chloro-6-fluorobenzoic acid (148 mg, 0.85 mmol). The mixture was stirred at ambient temperature for 12 hours. Water (3 ml) was added and the mixture was extracted with ethyl acetate and purified by flash chromatography eluting with 0 to 100% ethyl acetate in petroleum ether to give the methyl ester intermediate (230 mg, 0.45 mmol, 53%) which was redissolved in DMF (2.5 ml). Sodium hydroxide 6N (0.301 ml, 1.80 mmol) was added. The mixture was stirred at 25° C. for 2 hours and purified by C18 reverse phase chromatography (basic conditions) to afford the title compound as a white solid. (158 mg, 70%); Mass spectrum [M+H]⁺=496; ¹H NMR Spectrum (DMSOd6) 1.70-1.78 (m, 2H), 1.79-1.89 (m, 2H), 2.43 (t, 2H) 2.54 (t partially hidden by DMSOd5, 2H), 2.60 (t, 2H), 2.89 (dd, 1H), 3.11 (dd, 1H), 3.19-3.26 (m, 2H), 4.57 (ddd, 1H), 6.26 (d, 1H), 6.46 (s, 1H), 7.04 (d, 1H), 7.08 (d, 2H), 7.18 (d, 2H), 7.23 (dd, 1H), 7.29 (d, 1H), 7.43 (ddd, 1H), 8.93 (d, 1H).

The procedure described above for example 67.1 was repeated by coupling methyl 4-[3-(5,6,7,8-tetrahydro-1,8-naphthyridin-2-yl)propyl]-L-phenylalaninate with the acid shown in Table 20. Thus were obtained the compounds shown in Table 20:

TABLE 20

| | | | | Mass | |
|---|---|---|---|---|---|
| | | | | Ion | ¹H NMR Data (500 MHz |
| Example | Acid | Name | Yield % | [M + H]⁺ | DMSOd6) |
| 67.1 | 2-chloro-6- | N-[(2-chloro-6- | 70 | 496 | 1.70-1.78 (m, 2H), |
| | fluorobenzoic | fluorophenyl)carbonyl]- | | | 1.79-1.89 (m, 2H), |
| | acid | 4-[3- | | | 2.43 (t, 2H) 2.54 (t |
| | | (5,6,7,8- | | | partially hidden by |
| | | tetrahydro-1,8- | | | DMSOd5, 2H), |
| | | naphthyridin-2- | | | 2.60 (t, 2H), 2.89 (dd, |
| | | yl)propyl]-L- | | | 1H), 3.11 (dd, 1H), |
| | | phenylalanine | | | 3.19-3.26 (m, 2H), |
| | | | | | 4.57 (ddd, 1H), |
| | | | | | 6.26 (d, 1H), 6.46 (s, |
| | | | | | 1H), 7.04 (d, 1H), |
| | | | | | 7.08 (d, 2H), |
| | | | | | 7.18 (d, 2H), 7.23 (dd, |
| | | | | | 1H), 7.29 (d, 1H), |
| | | | | | 7.43 (ddd, 1H), |
| | | | | | 8.93 (d, 1H) |
| 67.2 | 2,6- | N-[2,6- | 42 | 512 | 1.70-1.78 (m, 2H), |
| | dichlorobenzoic | dichlorophenyl) | | | 1.79-1.89 (m, 2H), |
| | acid | carbonyl]-4-[3- | | | 2.43 (t, 2H), 2.53 (t |
| | | (5,6,7,8- | | | partially hidden by |
| | | tetrahydro-1,8- | | | DMSOd5, 2H), |
| | | naphthyridin-2- | | | 2.60 (t, 2H), 2.89 (dd, |
| | | yl)propyl]-L- | | | 1H), 3.10 (dd, 1H), |
| | | phenylalanine | | | 3.20-3.27 (m, 2H), |
| | | | | | 4.62 (ddd, 1H), |
| | | | | | 6.25 (d, 1H), 6.44 (bs, |
| | | | | | 1H), 7.04 (d, 1H), |
| | | | | | 7.07 (d, 2H), |
| | | | | | 7.19 (d, 2H), 7.37 (dd, |
| | | | | | 1H), 7.39-7.44 (m, |
| | | | | | 2H), 8.91 (d, 1H) |
| 67.3 | 2-chloro-4- | N-[(2-chloro-4- | 42 | 496 | 1.71-1.79 (m, 2H), |
| | fluorobenzoic | fluorophenyl)carbonyl]- | | | 1.80-1.92 (m, 2H), |
| | acid | 4-[3- | | | 2.43 (t, 2H), 2.55 (t, |
| | | (5,6,7,8- | | | 2H), 2.61 (t, 2H), |
| | | tetrahydro-1,8- | | | 2.92 (dd, 1H), |
| | | naphthyridin-2- | | | 3.15 (dd, 1H), |
| | | yl)propyl]-L- | | | 3.20-3.28 (m, 2H), 4.56 (ddd, |
| | | phenylalanine | | | 1H), 6.25 (d, 1H), |
| | | | | | 6.47 (bs, 1H), |
| | | | | | 7.04 (d, 1H), 7.11 (d, |
| | | | | | 2H), 7.20 (d, 2H), |
| | | | | | 7.22-7.31 (m, 2H), |
| | | | | | 7.45 (dd, 1H), |
| | | | | | 8.69 (d, 1H) |

The starting material was prepared as follows:

A suspension of methyl 4-bromo-N-(tert-butoxycarbonyl)-L-phenylalaninate (13 g, 36.29 mmol), bis(triphenylphosphine) palladium (II) chloride (1.53 g, 2.18 mmol), CuI (276 mg, 1.45 mmol), 4-pentyn-2-ol (5.11 ml, 54.4 mmol) and TEA (20.21 ml, 145 mmol) in DMF (140 ml) was stirred at 80° C. under nitrogen for 2 hours. The mixture was extracted with DCM and purified by silica gel flash chromatography (10 to 100% ethyl acetate in petroleum ether) to afford methyl N-(tert-butoxycarbonyl)-4-(4-hydroxypent-1-yn-1-yl)-L-phenylalaninate (10.3 g, 79%); ¹H NMR Spectrum (DMSOd6) 1.18 (d, 3H), 1.32 (s, 9H), 2.41 (dd, 1H), 2.52 (dd partially hidden by DMSOd5, 1H), 2.84 (dd, 1H), 2.98 (dd, 1H), 3.60 (s, 3H), 3.78-3.86 (m, 1H), 4.16 (ddd, 1H), 4.83 (d, 1H), 7.20 (d, 2H), 7.27-7.33 (m, 3H).

A mixture of methyl N-(tert-butoxycarbonyl)-4-(4-hydroxypent-1-yn-1-yl)-L-phenylalaninate (3 g, 8.3 mmol) and Pd/C 10% (300 mg) in methanol (100 ml) was hydrogenated under 4 bar of H₂ for 3 hours. Pd/C 10% (300 mg) was added and the mixture was hydrogenated further for 12 hours then filtered through celite, evaporated and purified by silica gel flash chromatography (50 to 100% ethyl acetate in petroleum ether) to afford methyl N-(tert-butoxycarbonyl)-4-(4-hydroxypentyl)-L-phenylalaninate (1.48 g, 49%); ¹H NMR Spectrum (DMSOd6) 1.01 (d, 3H), 1.25-1.39 (m, 2H), 1.32 (s, 9H), 1.47-1.56 (m, 1H), 1.57-1.66 (m, 1H), 2.45-2.56 (m partially hidden by DMSOd5, 2H), 2.80 (dd, 1H), 2.94 (dd, 1H), 3.53-3.61 (m, 1H), 3.60 (s, 3H), 4.13 (ddd, 1H), 4.33 (d, 1H), 7.08 (d, 2H), 7.12 (d, 2H), 7.27 (d, 1H).

A solution of methyl N-(tert-butoxycarbonyl)-4-(4-hydroxypentyl)-L-phenylalaninate (3.7 g, 10.12 mmol) and pyridinium dichromate (26.65 g, 71.1 mmol) in DCM (75 ml) was stirred at ambient temperature for 24 hours. After filtration of the insoluble and extraction with DCM, the residue was purified by silica gel flash chromatography (60 to 75% ethyl acetate in petroleum ether) to give methyl N-(tert-butoxycarbonyl)-4-(4-oxopentyl)-L-phenylalaninate (3.33 g, 90%); ¹H NMR Spectrum (DMSOd6) 1.32 (s, 9H), 1.67-1.77 (m, 2H), 2.06 (s, 3H), 2.40 (t, 2H), 2.49 (t partially hidden by DMSOd6, 2H), 2.80 (dd, 1H), 2.94 (dd, 1H), 3.60 (s, 3H), 4.14 (ddd, 1H), 7.08 (d, 2H), 7.13 (d, 2H), 7.27 (d, 1H).

To a solution of methyl N-(tert-butoxycarbonyl)-4-(4-oxopentyl)-L-phenylalaninate (1.25 g, 110.24 mmol) in ethanol (80 ml) were added 2-aminopyridine-3-carboxaldehyde (3.35 g, 9.21 mmol) and L-proline (0.589 g, 5.12 mmol). The mixture was stirred overnight under argon atmosphere. After evaporation, the residue was taken up in DCM, washed with saturated NaHCO₃ and purified by silica gel flash chromatography (80 to 100% ethyl acetate in petroleum ether) to afford methyl N-(tert-butoxycarbonyl)-4-[3-(1,8-naphthyridin-2-yl)propyl]-L-phenylalaninate (2.95 g, 64%); Mass spectrum [M+H]⁺=450; ¹H NMR Spectrum (DMSOd6) 1.31 (s, 9H), 2.03-2.14 (m, 2H), 2.65 (t, 2H), 2.81 (dd, 1H), 2.91-3.02 (m, 3H), 3.60 (ms, 3H), 4.14 (ddd, 1H), 7.15 (s, 4H), 7.28 (d, 1H), 7.54 (d, 1H), 7.57 (dd, 1H), 8.37 (d, 1H), 8.42 (dd, 1H), 9.03 (dd, 1H).

A suspension methyl N-(tert-butoxycarbonyl)-4-[3-(1,8-naphthyridin-2-yl)propyl]-L-phenylalaninate (2.95 g, 6.56 mmol) and Pd/C 10% (0.450 g) in EtOH (150 ml) was hydrogenated under 4 atmosphere for 4 hours. The resulting solution was filtered and the filtrate was concentrated to dryness and purified by silica gel flash chromatography (2% methanol in DCM ) to afford methyl N-(tert-butoxycarbonyl)-4-[3-(5,6,7,8-tetrahydro-1,8-naphthyridin-2-yl)propyl]-L-phenylalaninate (2.1 g, 71%); Mass spectrum [M+H]⁺=454; ¹H NMR Spectrum (DMSOd6) 1.31 (s, 9H), 1.70-1.78 (m, 2H), 1.79-1.89 (m, 2H), 2.42 (t, 2H) 2.52 (t partially hidden by DMSOd5, 2H), 2.60 (t, 2H), 2.80 (dd, 1H), 2.94 (dd, 1H), 3.19-3.26 (m, 2H), 3.60 (s, 3H), 4.13 (ddd, 1H), 6.24 (d, 1H), 6.25 (s, 1H), 7.01 (d, 1H), 7.09 (d, 2H), 7.13 (d, 2H), 7.27 (d, 1H).

TFA (13 ml) was added to a solution of methyl N-(tert-butoxycarbonyl)-4-[3-(5,6,7,8-tetrahydro-1,8-naphthyridin-2-yl)propyl]-L-phenylalaninate (1.5 g, 3.31 mmol) in dichloromethane (13 ml ). The solution was stirred at 25° C. for 2 h. The reaction mixture was concentrated to dryness, taken-up in DCM. An excess of a solution of NH3/MeOH 7N was added and the mixture was concentrated to dryness and purified by silica gel flash chromatography (5 to 20% methanol in DCM to afford methyl 4-bromo-N-(tert-butoxycarbonyl)-L-phenylalaninate (452 mg 97%) as a clear white foam. ¹H NMR Spectrum (DMSOd6) 1.74-1.84 (m, 2H), 1.84-1.95 (m, 2H), 2.54-2.62 (m, 4H), 2.68 (t, 2H), 3.03 (dd, 1H), 3.08 (dd, 1H), 3.31-3.37 (m partially hidden by H2O, 2H), 3.67 (s, 3H), 4.28 (t, 1H), 6.47 (d, 1H), 7.12 (d, 2H), 7.18 (d, 2H), 7.38 (d, 1H), 7.74 (ms, 1H), 8.98 (m, 2H).

### Example 68.1

### N-[(2-chlorophenyl)carbonyl]-3-{5-[3-(5,6,7,8-tetrahydro-1,8-naphthyridin-2-yl)propyl]pyridin-2-yl}-L-alanine

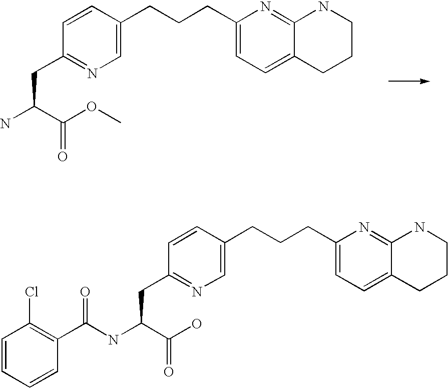

To a solution of methyl 3-{5-[3-(5,6,7,8-tetrahydro-1,8-naphthyridin-2-yl)propyl]pyridin-2-yl}-L-alaninate (300 mg, 0.85 mmol, described in Example 65) in DCM (5 ml) were added HOPO (121 mg, 1.09 mmol), EDCI (211 mg, 1.1 mmol) and 2-chlorobenzoic acid (133 mg, 0.85 mmol); The mixture was stirred at ambient temperature for 12 hours. Water (3 ml) was added. The organic phase was separated and purified by flash chromatography eluting with 40% ethyl acetate in petroleum ether to give an intermediate which was redissolved in DMF (2.5 ml). NaOH 6N (0.303 ml, 1.82 mmol) was added and the mixture was stirred at ambient temperature for 2 hours. Purification by C18 reverse phase chromatography (basic conditions) afforded the title compound (182 mg, 62%).

The procedure described above for example 68.1 was by coupling methyl 3-{5-[3-(5,6,7,8-tetrahydro-1,8-naphthyridin-2-yl)propyl]pyridin-2-yl}-L-alaninate with the acid shown in Table 21. Thus were obtained the compounds shown in Table 21

TABLE 21

| | | | | Mass | |
|---|---|---|---|---|---|
| | | | | Ion | ¹H NMR Data (500 MHz |
| Example | Acid | Name | Yield % | [M + H]⁺ | DMSOd6) |
| 68.1 | 2- | N-[(2- | 62 | 479 | 1.68-1.79 (m, 2H), |
| | chlorobenzoic | chlorophenyl)carbonyl]- | | | 1.81-1.92 (m, 2H), |
| | acid | 3-{5- | | | 2.43 (t, 2H), 2.57 (t, |
| | | [3-(5,6,7,8- | | | 2H), 2.60 (t, 2H), |
| | | tetrahydro-1,8- | | | 3.08 (dd, 1H), |
| | | naphthyridin- | | | 3.18-3.30 (m, 3H), |
| | | 2- | | | 4.80 (ddd, 1H), 6.25 (d, |
| | | yl)propyl]pyridin- | | | 1H), 6.44 (bs, 1H), |
| | | 2-yl}-L- | | | 7.04 (d, 1H), 7.22 (d, |
| | | alanine | | | 1H), 7.26 (d, 1H), |
| | | | | | 7.34 (ddd, 1H), |
| | | | | | 7.38-7.45 (m, 2H), |
| | | | | | 7.54 (dd, 1H), 8.34 (d, |
| | | | | | 1H), 8.64 (d, 1H) |
| 68.2 | 2-fluoro- | N-[(2-fluoro-6- | 51 | 477 | 1.71-1.79 (m, 2H), |
| | 6- | methylophenyl) | | | 1.83-1.92 (m, 2H), |
| | methylbenzoic | carbonyl]-3- | | | 2.05 (s, 3H), 2.45 (t, 2H), |
| | acid | {5-[3-(5,6,7,8- | | | 2.57 (t, 2H), 2.61 (t, |
| | | tetrahydro-1,8- | | | 2H), 3.03 (dd, 1H), |
| | | naphthyridin- | | | 3.21-3.30 (m, 3H), |
| | | 2- | | | 4.77 (ddd, 1H), 6.27 (d, 1H), |
| | | yl)propyl]pyridin- | | | 6.51 (s, 1H), |
| | | 2-yl}-L- | | | 6.94-7.01 (m, 2H), 7.05 (d, 1H), |
| | | alanine | | | 7.21 (d, 1H), 7.26 (ddd, |
| | | | | | 1H), 7.51 (dd, 1H), |
| | | | | | 8.33 (s, 1H), 8.54 (d, |
| | | | | | 1H) |
| 68.3 | 2-chloro- | N-[(2-chloro- | 53 | 497 | 1.71-1.78 (m, 2H), |
| | 4- | 4- | | | 1.82-1.91 (m, 2H), |
| | fluorobenzoic | fluorophenyl)carbonyl]- | | | 2.44 (t, 2H), 2.56 (t, |
| | acid | 3-{5- | | | 2H), 2.60 (t, 2H), |
| | | [3-(5,6,7,8- | | | 3.07 (dd, 1H), 3.24 (t, 2H), |
| | | tetrahydro-1,8- | | | 3.27 (dd, 1H), |
| | | naphthyridin- | | | 4.75 (ddd, 1H), 6.25 (d, |
| | | 2- | | | 1H), 6.57 (s, 1H), |
| | | yl)propyl]pyridin- | | | 7.04 (d, 1H), 7.19-7.27 (m, |
| | | 2-yl}-L- | | | 2H), 7.34 (dd, 1H), |
| | | alanine | | | 7.42 (dd, 1H), |
| | | | | | 7.52 (dd, 1H), 8.33 (d, 1H), |
| | | | | | 8.58 (d, 1H) |
| 68.4 | 2-chloro- | N-[(2-chloro- | 62 | 479 | 1.71-1.79 (m, 2H), |
| | 6- | 6- | | | 1.81-1.91 (m, 2H), |
| | fluorobenzoic | fluorophenyl)carbonyl]- | | | 2.44 (t, 2H), 2.56 (t, |
| | acid | 3-{5- | | | 2H), 2.60 (t, 2H), |
| | | [3-(5,6,7,8- | | | 3.03 (dd, 1H), |
| | | tetrahydro-1,8- | | | 3.20-3.28 (m, 3H), 4.82 (ddd, |
| | | naphthyridin- | | | 1H), 6.26 (d, 1H), |
| | | 2- | | | 6.45 (s, 1H), 7.04 (d, 1H), |
| | | yl)propyl]pyridin- | | | 7.18-7.24 (m, 2H), |
| | | 2-yl}-L- | | | 7.27 (d, 1H), |
| | | alanine | | | 7.41 (ddd, 1H), 7.51 (dd, |
| | | | | | 1H), 8.33 (d, 1H), |
| | | | | | 8.91 (d, 1H) |

### Example 69.1

### N-[(2,6-dichlorophenyl)carbonyl]-3-{6-[3-(5,6,7,8-tetrahydro-1,8-naphthyridin-2-yl)propyl]pyridin-3-yl}-L-alanine

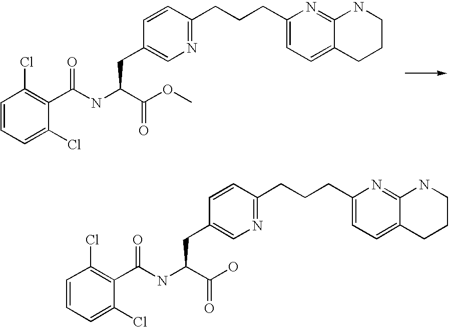

Methyl N-[(2,6-dichlorophenyl)carbonyl]-3-{6-[3-(5,6,7,8-tetrahydro-1,8-naphthyridin-2-yl)propyl]pyridin-3-yl}-L-alaninate was prepared using similar a procedure similar to that described in example 65, but replacing methyl 3-(5-bromopyridin-2-yl)-N-(tert-butoxycarbonyl)-L-alaninate by methyl 3-(6-bromopyridin-3-yl)-N-(tert-butoxycarbonyl)-L-alaninate.

Sodium hydroxide 6N (0.175 ml, 1.05 mmol) was added to a stirred solution of methyl N-[(2,6-dichlorophenyl)carbonyl]-3-{6-[3-(5,6,7,8-tetrahydro-1,8-naphthyridin-2-yl)propyl]pyridin-3-yl}-L-alaninate (185 mg, 0.35 mmol) in THF (2 ml) at 25° C. The resulting solution was stirred at 25° C. for 2 hours. Sodium hydroxide 6N (0.175 ml, 1.05 mmol) was further added. The mixture was stirred at 25° C. for 4 hours, concentrated, redissolved in DMF and purified by C18 reverse phase chromatography (basic conditions) to afford the title compound (140 mg, 78%).

The procedure described above for example 69.1 was repeated by coupling methyl 3-{6-[3-(5,6,7,8-tetrahydro-1,8-naphthyridin-2-yl)propyl]pyridin-3-yl}-L-alaninate with the appropriate acid shown in Table 22. Thus were obtained the compounds shown in Table 22:

TABLE 22

| | | | | Mass | |
|---|---|---|---|---|---|
| | | | | Ion | ¹H NMR Data (500 MHz |
| Example | acid | Name | Yield % | [M + H]⁺ | DMSOd6) |
| 69.1 | 2,6- | N-[(2,6- | 78 | 513 | 1.70-1.79 (m, 2H), |
| | dichlorobenzoic | dichlorophenyl)carbonyl]- | | | 1.88-1.99 (m, 2H), |
| | acid | 3-{6-[3- | | | 2.44 (t, 2H), 2.60 (t, |
| | | (5,6,7,8- | | | 2H), 2.66 (t, 2H), |
| | | tetrahydro-1,8- | | | 2.91 (dd, 1H), 3.13 (dd, |
| | | naphthyridin-2- | | | 1H), 3.23 (bs, 2H), |
| | | yl)propyl]pyridin- | | | 4.59-4.67 (m, 1H), |
| | | 3-yl}-L-alanine | | | 6.26 (d, 1H), 6.39 (s, |
| | | | | | 1H), 7.04 (d, 1H), |
| | | | | | 7.13 (d, 1H), 7.37 (dd, 1H), |
| | | | | | 7.40-7.44 (m, 2H), |
| | | | | | 7.60 (dd, 1H), 8.35 (d, |
| | | | | | 1H), 8.89 (d, 1H) |
| 69.2 | 2- | N-[(2- | 53 | 479 | 1.70-1.79 (m, 2H), |
| | chlorobenzoic | chlorophenyl)carbonyl]- | | | 1.89-1.99 (m, 2H), |
| | acid | 3-{6-[3- | | | 2.44 (t, 2H), 2.60 (t, |
| | | (5,6,7,8- | | | 2H), 2.67 (t, 2H), |
| | | tetrahydro-1,8- | | | 2.93 (dd, 1H), 3.17 (dd, |
| | | naphthyridin-2- | | | 1H), 3.21-3.26 (m, |
| | | yl)propyl]pyridin- | | | 2H), 4.58 (ddd, 1H), |
| | | 3-yl}-L-alanine | | | 6.25 (d, 1H), 6.59 (s, |
| | | | | | 1H), 7.04 (d, 1H), |
| | | | | | 7.15 (d, 1H) 7.22 (dd, 1H), |
| | | | | | 7.35 (ddd, 1H), |
| | | | | | 7.38-7.47 (m, 2H), 7.60 (dd, |
| | | | | | 1H), 8.36 (d, 1H), |
| | | | | | 8.65 (d, 1H) |
| 69.3 | 2- | N-[(2-chloro-4- | 42 | 497 | 1.70-1.79 (m, 2H), |
| | chloro- | fluorophenyl)carbonyl]- | | | 1.89-1.99 (m, 2H), |
| | 4- | 3-{6-[3- | | | 2.44 (t, 2H), 2.61 (t, |
| | fluorobenzoic | (5,6,7,8- | | | 2H), 2.68 (t, 2H), |
| | acid | tetrahydro-1,8- | | | 2.92 (dd, 1H), 3.17 (dd, |
| | | naphthyridin-2- | | | 1H), 3.21-3.27 (m, |
| | | yl)propyl]pyridin- | | | 2H), 4.59 (ddd, 1H), |
| | | 3-yl}-L-alanine | | | 6.25 (d, 1H), 6.57 (s, |
| | | | | | 1H), 7.05 (d, 1H), |
| | | | | | 7.15 (d, 1H), 7.24 (ddd, |
| | | | | | 1H), 7.28 (dd, 1H), |
| | | | | | 7.44 (dd, 1H), 7.60 (dd, |
| | | | | | 1H), 8.36 (d, 1H), |
| | | | | | 8.71 (d, 1H) |
| 69.4 | 2- | N-[(2-chloro-6- | 50 | 497 | 1.71-1.79 (m, 2H), |
| | chloro- | fluorophenyl)carbonyl]- | | | 1.89-1.98 (m, 2H), |
| | 6- | 3-{6-[3- | | | 2.45 (t, 2H), 2.61 (t, |
| | fluorobenzoic | (5,6,7,8- | | | 2H), 2.67 (t, 2H), |
| | acid | tetrahydro-1,8- | | | 2.91 (dd, 1H), 3.14 (dd, |
| | | naphthyridin-2- | | | 1H), 3.20-3.27 (m, |
| | | yl)propyl]pyridin- | | | 2H), 4.59-4.66 (m, |
| | | 3-yl}-L-alanine | | | 1H), 6.26 (d, 1H), |
| | | | | | 6.59 (bs, 1H), 7.05 (d, 1H), |
| | | | | | 7.14 (d, 1H), 7.23 (dd, |
| | | | | | 1H), 7.29 (dd, 1H), |
| | | | | | 7.43 (ddd, 1H), |
| | | | | | 7.59 (dd, 1H), 8.34 (d, 1H), |
| | | | | | 8.95 (d, 1H) |

The methyl 3-(6-bromopyridin-3-yl)-N-(tert-butoxycarbonyl)-L-alaninate used as the starting material was prepared as described below:

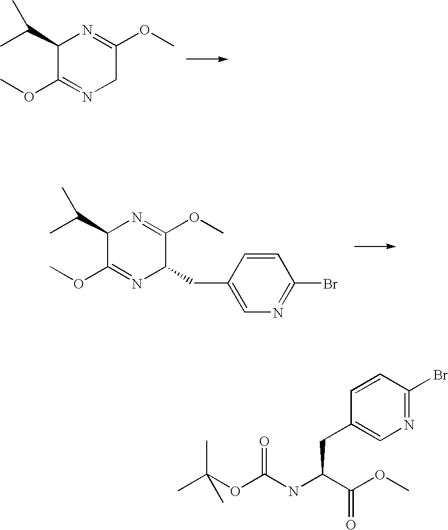

To a solution of (R)-2,5-dihydro-3,6-dimethoxy-2-isopropylpyrazine (5 ml, 27.9 mmol) (commercially available) in THF (130 ml) was added at −78° C. nBuLi 1.6M (17.44 ml, 27.9 mmol). The solution was stirred at −78° C. for 10 minutes and treated with a solution of 2-bromo-5-bromomethylpyridine (7 g, 27.9 mmol) in THF (2 ml). After stirring for 2 hours, the reaction mixture was allowed to warm up at room temperature, extracted with ether and purified by silica gel flash chromatography (10 to 30% ethyl acetate in petroleum ether) to give (2S,5R)-2-[(6-bromopyridin-3-yl)methyl]-3,6-dimethoxy-5-isopropyl-2,5-dihydropyrazine (8.22 g, 83%); ¹H NMR Spectrum (DMSOd6): 0.58 (d, 3H), 0.93 (d, 3H), 2.06-2.15 (m, 1H), 2.97 (dd, 1H), 3.04 (dd, 1H), 3.05 (t, 1H), 3.61 (s, 3H), 3.66 (s, 3H), 4.34-4.39 (m, 1H), 7.47 (dd, 1H), 7.54 (d, 1H), 8.11 (d, 1H).

A solution of HCl 0.25N (113 ml, 28.23 mmol) was added to a solution of (2S,5R)-2-[(6-bromopyridin-3-yl)methyl]-3,6-dimethoxy-5-isopropyl-2,5-dihydropyrazine (5 g, 14.11 mmol) in THF (20 ml). The solution was stirred at ambient temperature for 3 hours. The reaction mixture was washed with ether (50 ml) and the aqueous phase was concentrated to afford the crude intermediate as an oil which was redissolved in dioxan (100 ml)/water (3 ml). Triethylamine (5.65 ml, 40.52 mmol) and di-tert-butyldicarbonate (5.9 g, 27.02 mmol) were added. The resulting solution was stirred for 3 hours. The reaction mixture was extracted with DCM. After evaporation the residue was purified by silica gel flash chromatography (0 to 30% ethyl acetate in petroleum ether) to give methyl 3-(6-bromopyridin-3-yl)-N-(tert-butoxycarbonyl)-L-alaninate (4.28 g, 88%); ¹H NMR Spectrum (DMSOd6): 1.30 (s, 9H), 2.83 (dd, 1H), 3.04 (dd, 1H), 3.64 (s, 3H), 4.23 (ddd, 1H), 7.37 (d, 1H), 7.57 (d, 1H), 7.65 (dd, 1H), 8.25 (d, 1H).

1. A compound of formula I: 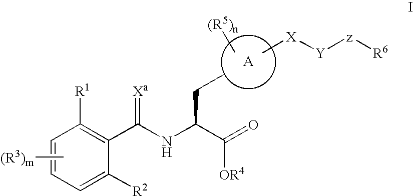
wherein:
X^{a} is selected from oxygen or sulphur;
R¹ is selected from bromo, chloro, (1-3C)alkyl, cyclopropyl, cyclobutyl, cyclopentyl, cyclopropylmethyl, cyclobutylmethyl, cyclopentylmethyl and halo-(1-3C)alkyl;
R² and each R³, which may be the same or different, is selected from hydrogen, halo, trifluoromethyl, cyano, isocyano, nitro, hydroxy, mercapto, amino, formyl, carboxy, carbamoyl, sulfamoyl, (1-6C)alkyl, (2-8C)alkenyl, (2-8C)alkynyl, (1-6C)alkoxy, (2-6C)alkenyloxy, (2-6C)alkynyloxy, (1-6C)alkylthio, (1-6C)alkylsulfinyl, (1-6C)alkylsulfonyl, (1-6C)alkylamino, di-[(1-6C)alkyl]amino, (1-6C)alkoxycarbonyl, N-(1-6C)alkylcarbamoyl, N,N-di-[(1-6C)alkyl]carbamoyl, (2-6C)alkanoyl, (2-6C)alkanoyloxy, (2-6C)alkanoylamino, N-(1-6C)alkyl-(2-6C)alkanoylamino, (3-6C)alkenoylamino, N-(1-6C)alkyl-(3-6C)alkenoylamino, (3-6C)alkynoylamino, N-(1-6C)alkyl-(3-6C)alkynoylamino, N-(1-6C)alkylsulfamoyl, N,N-di-[(1-6C)alkyl]sulfamoyl, (1-6C)alkanesulfonylamino and N-(1-6C)alkyl-(1-6C)alkanesulfonylamino,
or from a group of the formula:
-Q¹-X¹
wherein X¹ is a direct bond or is selected from O, S, SO, SO₂, N(R⁷), C(O), CH(OR⁷), C(O)N(R⁷), N(R⁷)C(O), SO₂N(R⁷), N(R⁷)SO₂, OC(R⁷)₂, SC(R⁷)₂ and N(R⁷)C(R⁷)₂, wherein R⁷ is hydrogen or (1-6C)alkyl, and Q¹ is aryl, aryl-(1-6C)alkyl, (3-7C)cycloalkyl, (3-7C)cycloalkyl-(1-6C)alkyl, (3-7C)cycloalkenyl, (3-7C)cycloalkenyl-(1-6C)alkyl, heteroaryl, heteroaryl-(1-6C)alkyl, heterocyclyl or heterocyclyl-(1-6C)alkyl,
and wherein R² and any R³ independently of each other optionally bears on carbon one or more R⁸ groups,
and wherein if any heteroaryl or heterocyclyl group within R² and any R³ contains an —NH— moiety, the nitrogen of said moiety optionally bears a group selected from R⁹,
and wherein any heterocyclyl group within R² and any R³ optionally bears 1 or 2 oxo or thioxo substituents;
or R² and an substituent optionally form a (1-3C)alkylenedioxy group;
or two R³ substituents optionally form a (1-3C)alkylenedioxy group;
m is 0, 1, 2 or 3;
R⁴ is selected from hydrogen, (1-6C)alkyl, heterocyclyl, heterocyclyl-(1-6C)alkyl, aryl, aryl-(1-6C)alkyl, heteroaryl and heteroaryl-(1-6C)alkyl, which optionally bears on carbon one or more R²¹ substituents, which may be the same or different,
and wherein if any heteroaryl or heterocyclyl group within R⁴ contains an —NH-moiety, the nitrogen of said moiety optionally bears a group selected from R²²,
and wherein and wherein any heterocyclyl group within R⁴ optionally bears 1 or 2 oxo or thioxo substituents; 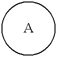
is selected from phenyl, pyridinyl and thiophenyl;
n is 0, 1, 2, 3 or 4, provided that when ring A is pyridinyl, n is 0, 1, 2 or 3 and that when ring A is thiophenyl, n is 0, 1 or 2;
each R⁵, which may be the same or different, is selected from halo, trifluoromethyl, cyano, isocyano, nitro, hydroxy, mercapto, amino, formyl, carboxy, carbamoyl, sulfamoyl, (1-6C)alkyl, (2-8C)alkenyl, (2-8C)alkynyl, (1-6C)alkoxy, (2-6C)alkenyloxy, (2-6C)alkynyloxy, (1-6C)alkylthio, (1-6C)alkylsulfinyl, (1-6C)alkylsulfonyl, (1-6C)alkylamino, di-[(1-6C)alkyl]amino, (1-6C)alkoxycarbonyl, N-(1-6C)alkylcarbamoyl, N,N-di-[(1-6C)alkyl]carbamoyl, (2-6C)alkanoyl, (2-6C)alkanoyloxy, (2-6C)alkanoylamino, N-(1-6C)alkyl-(2-6C)alkanoylamino, (3-6C)alkenoylamino, N-(1-6C)alkyl-(3-6C)alkenoylamino, (3-6C)alkynoylamino, N-(1-6C)alkyl-(3-6C)alkynoylamino, N-(1-6C)alkylsulfamoyl, N,N-di-[(1-6C)alkyl]sulfamoyl, (1-6C)alkanesulfonylamino and N-(1-6C)alkyl-(1-6C)alkanesulfonylamino,
or from a group of the formula:
-Q⁵-X⁷
wherein X⁷ is a direct bond or is selected from O, S, SO, SO₂, N(R²³), C(O), CH(OR²³), C(O)N(R²³), N(R²³)C(O), SO₂N(R²³), N(R²³)SO₂, OC(R²³)₂, SC(R²³)₂ and N(R²³)C(R²³)₂, wherein R²³ is hydrogen or (1-6C)alkyl, and Q⁵ is aryl, aryl-(1-6C)alkyl, (3-7C)cycloalkyl, (3-7C)cycloalkyl-(1-6C)alkyl, (3-7C)cycloalkenyl, (3-7C)cycloalkenyl-(1-6C)alkyl, heteroaryl, heteroaryl-(1-6C)alkyl, heterocyclyl or heterocyclyl-(1-6C)alkyl,
and wherein R⁵ optionally bears on carbon one or more R²⁴ groups,
and wherein any if any heteroaryl or heterocyclyl group within R⁵ contains an —NH— moiety, the nitrogen of said moiety optionally bears a group selected from R²⁵,
and wherein any heterocyclyl group within R⁵ optionally bears 1 or 2 oxo or thioxo substituents;
or two R⁵ substituents optionally form a (1-3C)alkylenedioxy group;
X is selected from a direct bond, N(R²⁶), O, S, SO, SO₂, C(O), CH(OR²⁶), C(O)N(R²⁶), N(R²⁶)C(O), SO₂N(R²⁶), N(R²⁶)SO₂, (1-6C)alkylene, CH═CH and C≡C, wherein R²⁶ is hydrogen, (1-6C)alkyl or (3-7C)cycloalkyl;
Y is selected from (1-6C)alkylene, (3-7C)cycloalkylene, (3-7C)cycloalkenylene and heterocyclyl,
Z is selected from a direct bond, N(R²⁶), O, S, SO, SO₂, C(O), CH(OR²⁶), SO₂N(R²⁶), N(R²⁶)SO₂, (1-6C)alkylene, CH═CH and C≡C, wherein R²⁶ is hydrogen, (1-6C)alkyl or (3-7C)cycloalkyl;
and wherein adjacent carbon atoms in any (2-6C)alkylene chain within an X, Y or Z substituent are optionally separated by the insertion into the chain of a group selected from O, S, SO, SO₂, N(R²⁷), C(O), CH(OR²⁷), C(O)N(R²⁷), N(R²⁷)C(O), SO₂N(R²⁷), N(R²⁷)SO₂, CH═CH and C≡C wherein R²⁷ is hydrogen, (1-6C)alkyl or (3-7C)cycloalkyl;
and wherein any X, Y or Z optionally bears on carbon one or more R²⁸ substituents,
and wherein if any heterocyclyl group within Y contains an —NH— moiety, the nitrogen of said moiety optionally bears a group selected from R²⁹;
provided that when X is a direct bond or O and Y is a heterocyclyl containing a nitrogen heteroatom, then the group R⁶-Z- is attached to a carbon atom in the heterocyclyl containing the nitrogen heteroatom;
R⁶ is heteroaryl, which heteroaryl contains at least one nitrogen atom, and wherein R⁶ optionally bears on carbon one or more R³¹ substituents,
and wherein if R⁶ contains an —NH— moiety, the nitrogen of said moiety optionally bears a group selected from R³⁵;
R⁸, R²¹, R²⁴ and R²⁸ is selected from halo, trifluoromethyl, cyano, nitro, hydroxy, amino, carboxy, carbamoyl, sulfamoyl, (1-6C)alkyl, (2-8C)alkenyl, (2-8C)alkynyl, (1-6C)alkoxy, (2-6C)alkenyloxy, (2-6C)alkynyloxy, (1-6C)alkylthio, (1-6C)alkylsulfinyl, (1-6C)alkylsulfonyl, (1-6C)alkylamino, di-[(1-6C)alkyl]amino, (1-6C)alkoxycarbonyl, N-(1-6C)alkylcarbamoyl, N,N-di-[(1-6C)alkyl]carbamoyl, (2-6C)alkanoyl, (2-6C)alkanoyloxy, (2-6C)alkanoylamino, N-(1-6C)alkyl-(2-6C)alkanoylamino, N-(1-6C)alkylsulfamoyl, N,N-di-[(1-6C)alkyl]sulfamoyl, (1-6C)alkanesulfonylamino and N-(1-6C)alkyl-(1-6C)alkanesulfonylamino,
or from a group of the formula:
—X²—R¹⁰
wherein X² is a direct bond or is selected from O, C(O) and N(R¹¹), wherein R¹¹ is hydrogen or (1-6C)alkyl, and R¹⁰ is halo-(1-6C)alkyl, hydroxy-(1-6C)alkyl, (1-6C)alkoxy-(1-6C)alkyl, cyano-(1-6C)alkyl, amino-(1-6C)alkyl, (1-6C)alkylamino-(1-6C)alkyl, di-[(1-6C)alkyl]amino-(1-6C)alkyl, (2-6C)alkanoylamino-(1-6C)alkyl and (1-6C)alkoxycarbonylamino-(1-6C)alkyl,
or from a group of the formula:
—X³-Q²
wherein X³ is a direct bond or is selected from O, S, SO, SO₂, N(R¹²), C(O), CH(OR¹²), C(O)N(R¹²), N(R¹²)C(O), SO₂N(R¹²), N(R¹²)SO₂, OC(R¹²)₂, SC(R¹²)₂ and N(R¹²)C(R¹²)₂, wherein R¹² is hydrogen or (1-6C)alkyl, and Q² is aryl, aryl-(1-6C)alkyl, (3-7C)cycloalkyl, (3-7C)cycloalkyl-(1-6C)alkyl, (3-7C)cycloalkenyl, (3-7C)cycloalkenyl-(1-6C)alkyl, heteroaryl, heteroaryl-(1-6C)alkyl, heterocyclyl or heterocyclyl-(1-6C)alkyl,
and wherein R⁸, R²¹, R²⁴ and R²⁸ independently of each other optionally bears on carbon one or more R¹³,
and wherein any if any heteroaryl or heterocyclyl group within R⁸, R²¹, R²⁴ and R²⁸ contains an —NH— moiety, the nitrogen of said moiety optionally bears a group selected from R¹⁴,
and wherein any heterocyclyl group within a substituent on R⁸, R²¹, R²⁴ and R²³ independently of each other optionally bears 1 or 2 oxo or thioxo substituents;
R⁹, R²², R²⁵ and R²⁹ are each independently selected from cyano, hydroxy, carboxy, carbamoyl, sulfamoyl, (1-6C)alkyl, (2-8C)alkenyl, (2-8C)alkynyl, (1-6C)alkylsulfonyl, (1-6C)alkoxycarbonyl, N-(1-6C)alkylcarbamoyl, N,N-di-[(1-6C)alkyl]carbamoyl, (2-6C)alkanoyl, N-(1-6C)alkylsulfamoyl and N,N-di-[(1-6C)alkyl]sulfamoyl,
or from a group of the formula:
—X⁴—R¹⁵
wherein X⁴ is a direct bond or is selected from C(O), SO₂, C(O)N(R¹⁶) and SO₂N(R¹⁶), wherein R¹⁶ is hydrogen or (1-6C)alkyl, and R¹⁵ is halo-(1-6C)alkyl, hydroxy-(1-6C)alkyl, (1-6C)alkoxy-(1-6C)alkyl, cyano-(1-6C)alkyl, amino-(1-6C)alkyl, (1-6C)alkylamino-(1-6C)alkyl, di-[(1-6C)alkyl]amino-(1-6C)alkyl, (2-6C)alkanoylamino-(1-6C)alkyl and (1-6C)alkoxycarbonylamino-(1-6C)alkyl,
or from a group of the formula:
—X⁵-Q³
wherein X⁵ is a direct bond or is selected from C(O), SO₂, C(O)N(R¹⁷) and SO₂N(R¹⁷), wherein R¹⁷ is hydrogen or (1-6C)alkyl, and Q³ is aryl, aryl-(1-6C)alkyl, (3-7C)cycloalkyl, (3-7C)cycloalkyl-(1-6C)alkyl, (3-7C)cycloalkenyl, (3-7C)cycloalkenyl-(1-6C)alkyl, heteroaryl, heteroaryl-(1-6C)alkyl, heterocyclyl or heterocyclyl-(1-6C)alkyl,
and wherein R⁹, R²², R²⁵ and R²⁹ independently of each other optionally bears on carbon one or more R¹⁸,
and wherein any if any heteroaryl or heterocyclyl group within R⁹, R²², R²⁵ and R²⁹ contains an —NH— moiety, the nitrogen of said moiety optionally bears a group selected from R¹⁹,
and wherein any heterocyclyl group within a substituent on R⁹, R²², R²⁵ and R²⁹ optionally bears 1 or 2 oxo or thioxo substituents;
R¹³ and R¹⁸ are each independently selected from halo, cyano, hydroxy, carboxy, amino, (3-6C)cycloalkyl, (2-6C)alkenyl, (2-6C)alkynyl, (1-6C)alkoxy, (1-6C)alkylamino and di-[(1-6C)alkyl]amino;
R¹⁴ and R¹⁹ are each independently selected from carbamoyl, sulfamoyl, (1-6C)alkyl, (2-8C)alkenyl, (2-8C)alkynyl, (1-6C)alkylsulfonyl, N-(1-6C)alkylcarbamoyl, N,N-di-[(1-6C)alkyl]carbamoyl, (2-6C)alkanoyl, N-(1-6C)alkylsulfamoyl and N,N-di-[(1-6C)alkyl]sulfamoyl,
or from a group of the formula:
—X⁶-Q⁴
wherein X⁶ is a direct bond or is selected from C(O), SO₂, C(O)N(R²⁰) and SO₂N(R²⁰), wherein R²⁰ is hydrogen or (1-6C)alkyl, and Q⁴ is (3-7C)cycloalkyl or (3-7C)cycloalkyl-(1-6C)alkyl;
R³¹ is selected from halo, cyano, hydroxy, nitro, amino, carbamoyl, sulfamoyl, (1-6C)alkyl, (2-6C)alkenyl, (2-6C)alkynyl, (1-6C)alkoxy, (2-6C)alkenyloxy, (2-6C)alkynyloxy, (1-6C)alkylthio, (1-6C)alkylsulfinyl, (1-6C)alkylsulfonyl, (1-6C)alkylamino, (2-8C)alkenylamino, (2-8C)alkynylamino, di-[(1-6C)alkyl]amino, (2-6C)alkanoyl, N-(1-6C)alkylcarbamoyl, N,N-di-[(1-6C)alkyl]carbamoyl, (1-6C)alkoxycarbonyl, (2-6C)alkanoyloxy, (2-6C)alkanoylamino, N-(1-6C)alkyl-(2-6C)alkanoylamino, N-(1-6C)alkylsulfamoyl, N,N-di-[(1-6C)alkyl]sulfamoyl, (1-6C)alkanesulfonylamino and N-(1-6C)alkyl-(1-6C)alkanesulfonylamino
or from a group of the formula:
—X⁸—R³²
wherein X⁸ is a direct bond or is selected from O, C(O) and N(R³³), wherein R³³ is hydrogen or (1-6C)alkyl, and R³² is halo-(1-6C)alkyl, hydroxy-(1-6C)alkyl, (1-6C)alkoxy-(1-6C)alkyl, amino-(1-6C)alkyl, (1-6C)alkylamino-(1-6C)alkyl, di-[(1-6C)alkyl]amino-(1-6C)alkyl, (2-6C)alkanoylamino-(1-6C)alkyl and (1-6C)alkoxycarbonylamino-(1-6C)alkyl,
or from a group of the formula:
—X⁹-Q⁶
wherein X⁹ is a direct bond or is selected from O, S, SO, SO₂, C(O), N(R³⁴), C(O)N(R³⁴), N(R³⁴)C(O), SO₂N(R³⁴), N(R³⁴)SO₂ wherein R³⁴ is hydrogen or (1-6C)alkyl, and Q⁶ is aryl, aryl-(1-6C)alkyl, (3-7C)cycloalkyl, (3-7C)cycloalkyl-(1-6C)alkyl, heteroaryl, heteroaryl-(1-6C)alkyl, heterocyclyl or heterocyclyl-(1-6C)alkyl,
and wherein R³¹ optionally bears on carbon one or more R³⁶,
and wherein any if any heteroaryl or heterocyclyl group within R³¹ contains an —NH— moiety, the nitrogen of said moiety optionally bears a group selected from R³⁷,
and wherein any heterocyclyl group within a substituent on R³¹ optionally bears 1 or 2 oxo or thioxo substituents;
R³⁵ and R³⁷ are each independently selected from (1-6C)alkyl, (2-8C)alkenyl, (2-8C)alkynyl, (1-6C)alkylsulfonyl and (2-6C)alkanoyl,
or from a group of the formula:
—X¹⁰-Q⁷
wherein X¹⁰ is a direct bond or is selected from C(O) and SO₂, wherein Q⁷ is (3-7C)cycloalkyl or (3-7C)cycloalkyl-(1-6C)alkyl;
R³⁶ is selected from halo, cyano, hydroxy, amino, (1-6C)alkyl, (2-6C)alkenyl, (2-6C)alkynyl, (3-6)cycloalkyl, (1-6C)alkoxy, (1-6C)alkylamino and di-[(1-6C)alkyl]amino,
provided that the compound of formula I is not 4-{[(2R)-2-amino-3-(3-pyridyl)-1-oxopropyl]amino}-N-[(2-ethyl-6-methylphenyl)thioxomethyl]-L-phenylalanine methyl ester;
or a pharmaceutically acceptable salt or prodrug thereof.

2. A compound according to claim 1 or a pharmaceutically acceptable salt thereof, wherein:
R¹ is selected from chloro, (1-3C)alkyl and halo-(1-3C)alkyl;
R⁴ is selected from hydrogen, (1-6C)alkyl, aryl, aryl-(1-6C)alkyl, heteroaryl and heteroaryl-(1-6C)alkyl, which optionally bears on carbon one or more R²¹ substituents, which may be the same or different,
and wherein if any heteroaryl group within R⁴ contains an —NH— moiety, the nitrogen of said moiety optionally bears a group selected from R²²,
X^{a} is oxygen;
R⁶ is heteroaryl, which heteroaryl contains at least one nitrogen atom, and wherein R⁶ optionally bears on carbon one or more R³¹ substituents,
and wherein if R⁶ contains an —NH— moiety, the nitrogen of said moiety optionally bears a group selected from R³⁵;
R³¹ is selected from halo, cyano, hydroxy, amino, (1-6C)alkyl, (2-6C)alkenyl, (2-6C)alkynyl, (1-6C)alkoxy, (1-6C)alkylamino and di-[(1-6C)alkyl]amino,
or from a group of the formula:
—X⁸—R³²
wherein X⁸ is a direct bond or is selected from O and N(R³³), wherein R³³ is hydrogen or (1-6C)alkyl, and R³² is halo-(1-6C)alkyl, hydroxy-(1-6C)alkyl, (1-6C)alkoxy-(1-6C)alkyl, amino-(1-6C)alkyl, (1-6C)alkylamino-(1-6C)alkyl and di-[(1-6C)alkyl]amino-(1-6C)alkyl,
or from a group of the formula:
—X⁹-Q⁶
wherein X⁹ is a direct bond or is selected from O and N(R³⁴), wherein R³⁴ is hydrogen or (1-6C)alkyl, and Q⁶ (3-7C)cycloalkyl or (3-7C)cycloalkyl-(1-6C)alkyl;
R³⁵ is selected from (1-6C)alkyl, (2-8C)alkenyl, (2-8C)alkynyl, (1-6C)alkylsulfonyl and (2-6C)alkanoyl,
or from a group of the formula:
—X¹⁰-Q⁷
wherein X¹⁰ is a direct bond or is selected from C(O) and SO₂, wherein Q⁷ is (3-7C)cycloalkyl or (3-7C)cycloalkyl-(1-6C)alkyl;
and
R², R³, R⁵, R²¹, R²², ring A, X, Y, Z, m and n are as defined in claim 1.

3. A compound according to claim 1 or a pharmaceutically acceptable salt thereof, wherein:
R¹ is selected from chloro, (1-3C)alkyl and halo-(1-3C)alkyl;
R⁴ is selected from hydrogen, (1-6C)alkyl, aryl, aryl-(1-6C)alkyl, heteroaryl and heteroaryl-(1-6C)alkyl, which optionally bears on carbon one or more R²¹ substituents, which may be the same or different,
and wherein if any heteroaryl group within R⁴ contains an —NH— moiety, the nitrogen of said moiety optionally bears a group selected from R²²;
R⁶ is heteroaryl, which heteroaryl contains at least one nitrogen atom, and wherein R⁶ optionally bears on carbon one or more R³¹ substituents,
and wherein if R⁶ contains an —NH— moiety, the nitrogen of said moiety optionally bears a group selected from R³⁵;
R³¹ is selected from halo, cyano, hydroxy, amino, (1-6C)alkyl, (2-6C)alkenyl, (2-6C)alkynyl, (1-6C)alkoxy, (1-6C)alkylamino and di-[(1-6C)alkyl]amino,
or from a group of the formula:
—X⁸—R³²
wherein X⁸ is a direct bond or is selected from O and N(R³³), wherein R³³ is hydrogen or (1-6C)alkyl, and R³² is halo-(1-6C)alkyl, hydroxy-(1-6C)alkyl, (1-6C)alkoxy-(1-6C)alkyl, amino-(1-6C)alkyl, (1-6C)alkylamino-(1-6C)alkyl and di-[(1-6C)alkyl]amino-(1-6C)alkyl,
or from a group of the formula:
—X⁹-Q⁶
wherein X⁹ is a direct bond or is selected from O and N(R³⁴), wherein R³⁴ is hydrogen or (1-6C)alkyl, and Q⁶ (3-7C)cycloalkyl or (3-7C)cycloalkyl-(1-6C)alkyl;
R³⁵ is selected from (1-6C)alkyl, (2-8C)alkenyl, (2-8C)alkynyl, (1-6C)alkylsulfonyl and (2-6C)alkanoyl,
or from a group of the formula:
—X¹⁰-Q⁷
wherein X¹⁰ is a direct bond or is selected from C(O) and SO₂, wherein Q⁷ is (3-7C)cycloalkyl or (3-7C)cycloalkyl-(1-6C)alkyl; and
R², R³, R⁵, R²¹, R²², ring A, X, X^{a}, Y, Z, m and n are as defined in claim 1.

4. A compound according to claim 1 or a pharmaceutically acceptable salt thereof, wherein R⁶ is linked to Z by a carbon atom in an aromatic ring in the heteroaryl group represented by R⁶ and wherein R⁶ is selected from any one of (a) to (f):
(a) imidazole which is substituted in an ortho position to the —N═ of the imidazole ring by —NHR^{31a};
(b) imidazole fused to:
(bi) a monocyclic 6-membered aromatic,
(bii) a monocyclic 5- or 6-membered heteroaromatic,
(biii) a 3 to 7-membered heterocyclic or
(biv) a (3-6C)cycloalkane ring,
and wherein R⁶ is substituted in the ortho position to the —N═ of the imidazole ring by —NHR^{31a};
(c) imidazole fused to a 5-, 6 or 7-membered heterocyclic, or to a monocyclic 5- or 6-membered heteroaromatic which heterocyclic or heteroaromatic contains at least one unsubstituted —NH— ring member and optionally contains 1 or 2 additional hetero atoms selected from O, S and N, and wherein the unsubstituted —NH— of the heterocyclic or heteroaromatic ring and the ═N— of the imidazole in R⁶ are attached to the same bridgehead ring atom at a junction of the two fused rings;
(d) pyridine which is substituted in an ortho position to the —N═ of the pyridine ring by —NHR^{31a};
(e) pyridine fused to:
(bi) a monocyclic 6-membered aromatic,
(bii) a monocyclic 5- or 6-membered heteroaromatic,
(biii) a 3 to 7-membered heterocyclic or
(biv) a (3-6C)cycloalkane ring,
and wherein R⁶ is substituted in an ortho position to the —N═ of the pyridine ring by —NHR^{31a}; and
(f) pyridine fused to a 5-, 6 or 7-membered heterocyclic, or to a monocyclic 5- or 6-membered heteroaromatic which heterocyclic or heteroaromatic contains at least one unsubstituted —NH— ring member and optionally contains 1 or 2 additional hetero atoms selected from O, S and N, and wherein the unsubstituted —NH— of the heterocyclic ring and the ═N— of the pyridine ring in R⁶ are attached to the same bridgehead ring atom at a junction of the two fused rings;
or the group Z is NH, R⁶ is attached to Z by a carbon atom in an aromatic ring in R⁶ that is ortho to a —N═ ring atom in R⁶ and wherein R⁶ is selected from any one of (i) to (iiii):
(i) imidazole;
(ii) imidazole fused to a 6-membered monocyclic aromatic, a 3- to 7-membered monocyclic heteroaromatic or heterocyclic ring or to a (3-6C)cycloalkane ring; and
(iii) pyridine fused 5- or 6-membered monocyclic aromatic, a 3- to 7-membered monocyclic heteroaromatic or heterocyclic ring or to a (3-6C)cycloalkane ring;
and wherein R⁶ optionally bears on carbon one or more R³¹ substituents,
and wherein if R⁶ contains an —NH— ring member, the nitrogen of said —NH— group optionally bears a group selected from R³⁵ (provided said —NH— group is not specified to be an unsubstituted —NH— above);
R^{31a} is selected from hydrogen, (1-6C)alkyl, (2-8C)alkenyl, (2-8C)alkynyl, halo-(1-6C)alkyl, hydroxy-(2-6C)alkyl, (1-6C)alkoxy-(2-6C)alkyl, amino-(2-6C)alkyl, (1-6C)alkylamino-(2-6C)alkyl, di-[(1-6C)alkyl]amino-(2-6C)alkyl, (3-7C)cycloalkyl and (3-7C)cycloalkyl-(1-6C)alkyl,
and wherein any (3-7C)cycloalkyl in R^{31a} optionally bears 1 or more (1-6C)alkyl substituents; and
wherein R³¹ and R³⁵ are as defined in claim 1.

5. A compound according to claim 1 or a pharmaceutically acceptable salt thereof, wherein R⁶ is: 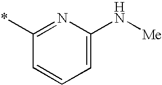
wherein * indicates the point of attachment of R⁶ to the group Z in formula I.

6. A compound according to claim 1 or a pharmaceutically acceptable salt thereof, wherein R⁶ is: 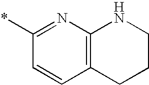
wherein * indicates the point of attachment of R⁶ to the group Z in formula I.

7. A compound according to claim 1 or a pharmaceutically acceptable salt thereof, wherein m is 0, 1 or 2 and each R³, which may be the same or different, is selected from halo, trifluoromethyl, hydroxy, amino, (1-6C)alkyl, (2-8C)alkenyl, (2-8C)alkynyl, (1-6C)alkoxy, (1-6C)alkylsulfonyl, (1-6C)alkylamino, di-[(1-6C)alkyl]amino, (1-6C)alkoxycarbonyl, (2-6C)alkanoyl, (2-6C)alkanoyloxy, amino-(1-6C)alkyl, (1-6C)alkylamino-(1-6C)alkyl and di-[(1-6C)alkyl]amino-(1-6C)alkyl,
or two R³ substituents optionally form a (1-3C)alkylenedioxy group.

8. A compound according to claim 1 or a pharmaceutically acceptable salt thereof, wherein ring A is pyridinyl.

9. A compound according to claim 8 or a pharmaceutically acceptable salt thereof, wherein ring A is: 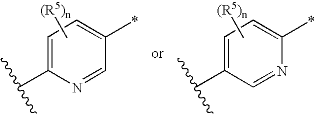
wherein * shows the point of attachment to X.

10. A compound according to claim 1 or a pharmaceutically acceptable salt thereof, wherein ring A is thiophenyl.

11. A compound according to claim 10 or a pharmaceutically acceptable salt thereof, wherein ring A is:

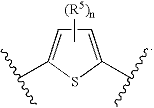

12. A compound according to claim 1 or a pharmaceutically acceptable salt thereof, wherein ring A is: 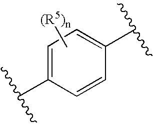
wherein n and R⁵ are as defined in claim 1.

13. A compound according to claim 1 wherein n is 0, 1 or 2 and each R⁵, which may be the same or different, is selected from halo, hydroxy, amino, (1-4C)alkyl, (1-4C)alkoxy, (1-4C)alkylamino and di-[(1-4C)alkyl]amino, and wherein R⁵ optionally bears on carbon one or more R²⁴ substituents selected from halo, hydroxy, amino, (1-4C)alkoxy, (1-4C)alkylamino and di-[(1-4C)alkyl]amino.

14. A compound according to claim 1 or a pharmaceutically acceptable salt thereof, wherein n is 0, 1 or 2 and each R⁵, which may be the same or different, is selected from halo, amino, (1-4C)alkyl, (1-4C)alkoxy, (1-4C)alkylamino and di-[(1-4C)alkyl]amino, and wherein R⁵ optionally bears on carbon one or more R²⁴ substituents selected from halo, hydroxy, amino, (1-4C)alkoxy, (1-4C)alkylamino and di-[(1-4C)alkyl]amino.

15. A compound according to claim 1 or a pharmaceutically acceptable salt thereof, wherein n is 0.

16. A compound according to claim 1 or a pharmaceutically acceptable salt thereof, wherein X and Z, which may be the same or different, are selected from a direct bond, N(R²⁶) , O, (1-6)alkylene, CH═CH and C≡C, wherein R²⁶ is hydrogen, (1-6C)alkyl or (3-7C)cycloalkyl.

17. A compound according to claim 1 or a pharmaceutically acceptable salt thereof, wherein X is selected from a direct bond and O.

18. A compound according to claim 1 or a pharmaceutically acceptable salt thereof, wherein X is a direct bond.

19. A compound according to claim 1 or a pharmaceutically acceptable salt thereof, wherein Y is (1-4C)alkylene, and wherein Y optionally bears on carbon one or more R²⁸ substituents selected from (1-3C)alkyl.

20. A compound according to claim 1 or a pharmaceutically acceptable salt thereof, wherein:
X is selected from a direct bond and O;
Y is selected from (1-4C)alkylene, and wherein Y optionally bears on carbon one or more R²⁸ substituents selected from (1-3C)alkyl; and
Z is selected from a direct bond, NR²⁶, wherein R²⁶ is hydrogen or (1-3C)alkyl.

21. A compound according to claims 1 or a pharmaceutically acceptable salt thereof, wherein the chain length between ring A and a ring —N═ in R⁶ is 4 or 5 atoms long,
or when Z is NH and R⁶ is attached to Z by a ring carbon atom in an ortho position to a —N═ atom in R⁶, the chain length of the group —X—Y-Z- is 4 or 5 atoms long.

22. A compound according to claim 1, or a pharmaceutically acceptable salt thereof, wherein the group —X—Y-Z- is selected from —(CH₂)₃—, *—O—(CH₂)₂— and *—O—(CH₂)₃—NH—;
wherein * represents the point of attachment to ring A in formula I;
and wherein the group X-Z-Y optionally bears on carbon one or more (1-3C)alkyl substituent.

23. A compound according to claim 1, or a pharmaceutically acceptable salt thereof, wherein the group —X—Y-Z- is —(CH₂)₃—,
and wherein the group X-Z-Y optionally bears on carbon one or more (1-3C)alkyl substituent.

24. A compound according to claim 1, or a pharmaceutically acceptable salt thereof, wherein the group —X—Y-Z- is *—O—(CH₂)₂—;
wherein * represents the point of attachment to ring A in formula I;
and wherein the group X-Z-Y optionally bears on carbon one or more (1-3C)alkyl substituent.

25. A compound according to claim 1 wherein R⁶ is linked to Z by a carbon atom in an aromatic ring in the heteroaryl group represented by R⁶.

26. A compound according to claim 1 wherein R⁶ is attached to the group Z in a position ortho to a nitrogen atom in R⁶.

27. A compound according to claim 1 or a pharmaceutically acceptable salt thereof, wherein R² is selected from hydrogen, fluoro, chloro, bromo and methyl.

28. A compound according to claim 1 or a pharmaceutically acceptable salt thereof, wherein R¹ is chloro or methyl and R² is selected from fluoro, chloro and methyl.

29. A compound according to claim 1 or a pharmaceutically acceptable salt thereof, wherein R¹ is chloro and R² is selected from hydrogen, fluoro, chloro and methyl.

30. A compound according to claim 1 or a pharmaceutically acceptable salt thereof, wherein R¹ is methyl and R² is selected from hydrogen, fluoro and chloro.

31. A compound according to claim 1, or a pharmaceutically acceptable salt thereof, wherein the group: 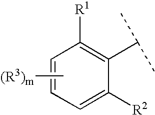
in formula I is selected from 2-acetylamino-6-chlorophenyl, 4-acetylamino-2-methylphenyl, 2-bromo-6-chlorophenyl, 2-bromo-4,5-difluorophenyl, 2-bromo-4-fluorophenyl, 2-bromo-5-fluorophenyl, 2-chlorophenyl, 2-chloro-3,6-difluorophenyl, 2-chloro-3,4-dimethoxyphenyl, 2-chloro-4,5-dimethoxyphenyl, 2-chloro-6-fluorophenyl, 2-chloro-4-fluorophenyl, 2-chloro-3-fluorophenyl, 2-chloro-5-fluorophenyl, 2-chloro-4-fluoro-5-sulfamoylphenyl, 3-chloro-2-fluoro-6-trifluoromethylphenyl, 6-chloro-2-fluoro-3-methylphenyl, 2-chloro-6-fluoro-3-methylphenyl, 6-chloro-2-fluoro-3-methylphenyl, 2-chloro-4-methoxyphenyl, 2-chloro-5-methoxyphenyl, 2-chloro-4-methylsulfonylphenyl, 2-chloro-5-methylphenyl, 2-chloro-3-methylphenyl, 2-chloro-6-methylphenyl, 4-chloro-2-methylphenyl, 5-chloro-2-methylphenyl, 3-chloro-2-methylphenyl, 2-chloro-4-morpholin-4-ylphenyl, 2-chloro-4-pyrrolidin-1-ylphenyl, 2-chloro-4-(1H-pyrazol-1-yl)phenyl, 2-chloro-3,6-difluorophenyl, 2-chloro-4,5-difluorophenyl, 2-chloro-5-trifluoromethylphenyl, 5-chloro-2-trifluoromethylphenyl, 2-chloro-3-trifluoromethylphenyl, 2-cyclopropyl-4-fluorophenyl, 3,6-dichloro-2-methoxyphenyl, 2,4-dichlorophenyl, 2,3-dichlorophenyl, 2,6-dichlorophenyl, 4,5-difluoro-2-methylphenyl, 2,3-dimethylphenyl, 2,4-dimethylphenyl, 2,6-dimethylphenyl, 2,5-dimethylphenyl, 2,4-dichloro-5-fluorophenyl, 2,6-dichloro-3-methylphenyl, 2-ethylphenyl, 2-ethyl-4-fluorophenyl, 2-ethyl-5-fluorophenyl, 3-fluoro-2-methylphenyl, 5-fluoro-2-methylphenyl, 4-fluoro-2-methylphenyl, 2-fluoro-6-methylphenyl, 2-fluoro-6-trifluoromethylphenyl, 3-fluoro-2-trifluoromethylphenyl, 5-fluoro-2-trifluoromethylphenyl, 4-fluoro-2-trifluoromethylphenyl, 3-hydroxy-2-methylphenyl, 2-methylphenyl, 2-methyl-3-trifluoromethylphenyl, 2-methyl-5-trifluoromethylphenyl, 4-methoxy-2-methylphenyl, 3-methoxy-2-methylphenyl, 4-methoxy-2-trifluoromethylphenyl, 5-chloro-1,3-benzodioxol-4-yl 2-trifluoromethylphenyl, 2,4,6-trimethylphenyl and 2,4,5-trimethylphenyl.

32. A compound according to claim 1 or a pharmaceutically acceptable salt thereof, wherein the group: 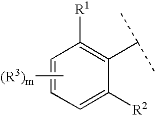
in formula I is selected from 2,6-dichlorophenyl, 2-chlorophenyl, 2-chloro-4-fluorophenyl, 2-chloro-6-fluorophenyl, 2-chloro-6-methylphenyl, 2-fluoro-6-methylphenyl, 2-chloro-4-methoxyphenyl, 2-ethyl-4-fluorophenyl and 2,6-dimethylphenyl.

33. A compound according to claim 1 or a pharmaceutically acceptable salt thereof, wherein X^{a} is oxygen.

34. A compound according to claim 1 or a pharmaceutically acceptable salt thereof, wherein R⁴ is hydrogen.

35. A compound according to claim 1 or a pharmaceutically acceptable salt thereof wherein:
Ring A is: 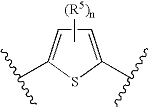
n is 0, 1 or 2;
each R⁵, which may be the same or different, is selected from halo, hydroxy, amino, (1-4C)alkyl, (1-4C)alkoxy (1-4C)alkylamino and di-[(1-4C)alkyl]amino, and wherein R⁵ optionally bears on carbon one or more R²⁴ substituents selected from halo, hydroxy, amino, (1-4C)alkoxy, (1-4C)alkylamino and di-[(1-4C)alkyl]amino;
the group —X—Y-Z- is —(CH₂)₃—,
and wherein the group X-Z-Y optionally bears on carbon one or more substituent selected from (1-3C)alkyl; and
R⁶ is selected from 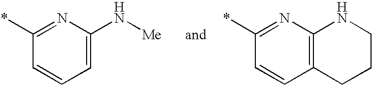
or a pharmaceutically acceptable salt thereof.

36. A compound according to claim 35 or a pharmaceutically acceptable salt thereof, wherein X^{a} is O.

37. A compound according to claim 35 or a pharmaceutically acceptable salt thereof, wherein R⁴ is hydrogen.

38. A compound according to claim 35 or a pharmaceutically acceptable salt thereof, wherein n is 0.

39. A compound according to claim 35 or a pharmaceutically acceptable salt thereof, wherein R⁴ is hydrogen, X^{a} is O and the group: 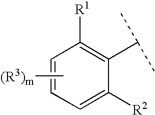
in formula I is selected from 2,6-dichlorophenyl, 2-chlorophenyl, 2-chloro-4-fluorophenyl, 2-chloro-6-fluorophenyl, 2-chloro-6-methylphenyl, 2-fluoro-6-methylphenyl, 2-chloro-4-methoxyphenyl, 2-ethyl-4-fluorophenyl and 2,6-dimethylphenyl.

40. A compound according to claim 1 or a pharmaceutically acceptable salt thereof wherein:
Ring A is: 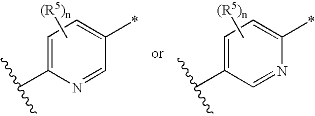
wherein * shows the point of attachment to X;
n is 0, 1, 2 or 3;
each R⁵, which may be the same or different, is selected from halo, hydroxy, amino, (1-4C)alkyl, (1-4C)alkoxy (1-4C)alkylamino and di-[(1-4C)alkyl]amino, and wherein R⁵ optionally bears on carbon one or more R²⁴ substituents selected from halo, hydroxy, amino, (1-4C)alkoxy, (1-4C)alkylamino and di-[(1-4C)alkyl]amino;
the group —X—Y-Z- is selected from —(CH₂)₃— and **—O—(CH₂)₂—;
wherein ** represents the point of attachment to the pyrindinyl ring in formula I,
and wherein the group X-Z-Y optionally bears on carbon one or more (1-3C)alkyl substituent; and
R⁶ is selected from: 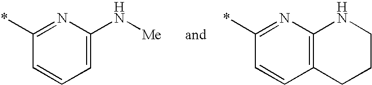
or a pharmaceutically acceptable salt thereof.

41. A compound according to claim 40 or a pharmaceutically acceptable salt thereof, wherein X^{a} is O.

42. A compound according to claim 40 or a pharmaceutically acceptable salt thereof, wherein R⁴ is hydrogen.

42. A compound according to claim 40 or a pharmaceutically acceptable salt thereof, wherein n is 0.

43. A compound according to claim 40 or a pharmaceutically acceptable salt thereof, wherein R⁴ is hydrogen, X^{a} is O and the group: 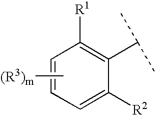
in formula I is selected from 2,6-dichlorophenyl, 2-chlorophenyl, 2-chloro-4-fluorophenyl, 2-chloro-6-fluorophenyl, 2-chloro-6-methylphenyl, 2-fluoro-6-methylphenyl, 2-chloro-4-methoxyphenyl, 2-ethyl-4-fluorophenyl and 2,6-dimethylphenyl.

44. A compound according to claim 1 or a pharmaceutically acceptable salt thereof wherein:
Ring A is: 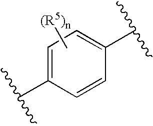
n is 0, 1, 2, 3 or 4;
each R⁵, which may be the same or different, is selected from halo, hydroxy, amino, (1-4C)alkyl, (1-4C)alkoxy (1-4C)alkylamino and di-[(1-4C)alkyl]amino, and wherein R⁵ optionally bears on carbon one or more R²⁴ substituents selected from halo, hydroxy, amino, (1-4C)alkoxy, (1-4C)alkylamino and di-[(1-4C)alkyl]amino;
the group —X—Y-Z- is selected from —(CH₂)₃— and *—O—(CH₂)₂—;
wherein * represents the point of attachment to ring A in formula I,
and wherein the group X-Z-Y optionally bears on carbon one or more substituent selected from (1-3C)alkyl; and
R⁶ is selected from: 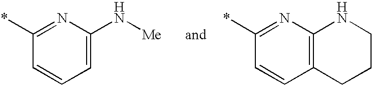
or a pharmaceutically acceptable salt thereof.

45. A compound according to claim 44 or a pharmaceutically acceptable salt thereof, wherein X^{a} is O.

46. A compound according to claim 44 or a pharmaceutically acceptable salt thereof, wherein R⁴ is hydrogen.

47. A compound according to claim 44 or a pharmaceutically acceptable salt thereof, wherein n is 0.

48. A compound according to claim 44 or a pharmaceutically acceptable salt thereof, wherein R⁴ is hydrogen, X^{a} is O and the group: 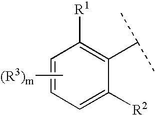
in formula I is selected from 2,6-dichlorophenyl, 2-chlorophenyl, 2-chloro-4-fluorophenyl, 2-chloro-6-fluorophenyl, 2-chloro-6-methylphenyl, 2-fluoro-6-methylphenyl, 2-chloro-4-methoxyphenyl, 2-ethyl-4-fluorophenyl and 2,6-dimethylphenyl.

49. A compound of formula I as defined in claim 1 wherein
R¹ is selected from chloro, methyl and ethyl;
R² is selected from hydrogen, fluoro, chloro, bromo and methyl;
m is 0 or 1 and R³ is selected from halo, (1-3C)alkyl and (1-3C)alkoxy,
or R² and an adjacent R³ substituent optionally form a methylenedioxy group;
Ring A is selected from: 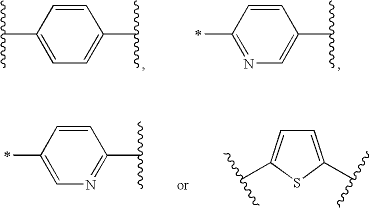
wherein * shows the point of attachment to X in formula I and wherein ring A is optionally substituted by n R⁵ groups;
X^{a} is oxygen or sulfur;
R⁴ is hydrogen;
n is 0, 1 or 2 and each R⁵, which may be the same or different, is selected from halo, amino, (1-4C)alkyl, (1-4C)alkoxy (1-4C)alkylamino and di-[(1-4C)alkyl]amino; the group —X—Y-Z- is selected from —(CH₂)₃— and *—O—(CH₂)₂—,
wherein * represents the point of attachment to ring A in formula I, provided that when ring A is: 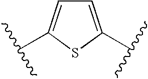
then —X—Y-Z- is —(CH₂)₃—;
and wherein the group X-Z-Y optionally bears on carbon one or more substituent selected from (1-3C)alkyl; and
R⁶ is selected from: 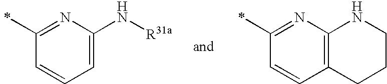
wherein R^{31a} is as defined in claim 4;
* indicates the point of attachment of R⁶ to the group Z in formula I;
and wherein R⁶ is optionally substituted on carbon by R³¹ as defined in claim 1;
or a pharmaceutically acceptable salt thereof.

50. A compound according to claim 49 or a pharmaceutically acceptable salt thereof, wherein:
R^{31a} is selected from hydrogen (1-3C)alkyl, (3-6C)cycloalkyl and (3-6C)cycloalkyl-(1-3C)alkyl; and
and wherein R⁶ is optionally substituted on carbon by R³¹ selected from (1-3C)alkyl, (3-6C)cycloalkyl and (3-6C)cycloalkyl-(1-3C)alkyl.

51. A compound according to claim 49 or a pharmaceutically acceptable salt thereof, wherein R⁶ is selected from: 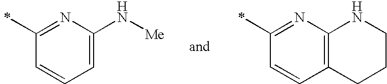
wherein * indicates the point of attachment of R⁶ to the group Z in formula I.

52. A compound according to claim 1 or a pharmaceutically acceptable salt thereof, wherein:
X^{a} is oxygen;
R⁴ is hydrogen;
Ring A is 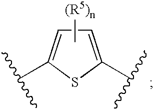
n is 0, 1 or 2;
—X—Y-Z- is —(CH₂)₃—;
and wherein the group X-Z-Y optionally bears on carbon one or more substituent selected from (1-3C)alkyl;
R⁶ is 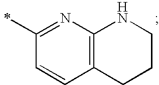
and
R¹R², R³, R⁵ m and n are as defined in claim 1.

53. A compound according to claim 52 or a pharmaceutically acceptable salt thereof, wherein R¹ is chloro or methyl and R² is selected from fluoro, chloro and methyl.

54. A compound according to claim 52 or a pharmaceutically acceptable salt thereof, wherein R¹ is chloro and R² is selected from hydrogen, fluoro, chloro and methyl.

55. A compound according to claim 52 or a pharmaceutically acceptable salt thereof, wherein R¹ is methyl and R² is selected from hydrogen, fluoro and chloro.

56. A compound according to claim 52, or a pharmaceutically acceptable salt thereof, wherein the group: 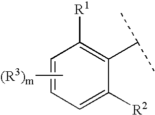
in formula I is selected from 2-acetylamino-6-chlorophenyl, 4-acetylamino-2-methylphenyl, 2-bromo-6-chlorophenyl, 2-bromo-4,5-difluorophenyl, 2-bromo-4-fluorophenyl, 2-bromo-5-fluorophenyl, 2-chlorophenyl, 2-chloro-3,6-difluorophenyl, 2-chloro-3,4-dimethoxyphenyl, 2-chloro-4,5-dimethoxyphenyl, 2-chloro-6-fluorophenyl, 2-chloro-4-fluorophenyl, 2-chloro-3-fluorophenyl, 2-chloro-5-fluorophenyl, 2-chloro-4-fluoro-5-sulfamoylphenyl, 3-chloro-2-fluoro-6-trifluoromethylphenyl, 6-chloro-2-fluoro-3-methylphenyl, 2-chloro-6-fluoro-3-methylphenyl, 6-chloro-2-fluoro-3-methylphenyl, 2-chloro-4-methoxyphenyl, 2-chloro-5-methoxyphenyl, 2-chloro-4-methylsulfonylphenyl, 2-chloro-5-methylphenyl, 2-chloro-3-methylphenyl, 2-chloro-6-methylphenyl, 4-chloro-2-methylphenyl, 5-chloro-2-methylphenyl, 3-chloro-2-methylphenyl, 2-chloro-4-morpholin-4-ylphenyl, 2-chloro-4-pyrrolidin-1-ylphenyl, 2-chloro-4-(1H-pyrazol-1-yl)phenyl, 2-chloro-3,6-difluorophenyl, 2-chloro-4,5-difluorophenyl, 2-chloro-5-trifluoromethylphenyl, 5-chloro-2-trifluoromethylphenyl, 2-chloro-3-trifluoromethylphenyl, 2-cyclopropyl-4-fluorophenyl, 3,6-dichloro-2-methoxyphenyl, 2,4-dichlorophenyl, 2,3-dichlorophenyl, 2,6-dichlorophenyl, 4,5-difluoro-2-methylphenyl, 2,3-dimethylphenyl, 2,4-dimethylphenyl, 2,6-dimethylphenyl, 2,5-dimethylphenyl, 2,4-dichloro-5-fluorophenyl, 2,6-dichloro-3-methylphenyl, 2-ethylphenyl, 2-ethyl-4-fluorophenyl, 2-ethyl-5-fluorophenyl, 3-fluoro-2-methylphenyl, 5-fluoro-2-methylphenyl, 4-fluoro-2-methylphenyl, 2-fluoro-6-methylphenyl, 2-fluoro-6-trifluoromethylphenyl, 3-fluoro-2-trifluoromethylphenyl, 5-fluoro-2-trifluoromethylphenyl, 4-fluoro-2-trifluoromethylphenyl, 3-hydroxy-2-methylphenyl, 2-methylphenyl, 2-methyl-3-trifluoromethylphenyl, 2-methyl-5-trifluoromethylphenyl, 4-methoxy-2-methylphenyl, 3-methoxy-2-methylphenyl, 4-methoxy-2-trifluoromethylphenyl, 5-chloro-1,3-benzodioxol-4-yl 2-trifluoromethylphenyl, 2,4,6-trimethylphenyl and 2,4,5-trimethylphenyl.

57. A compound according to claim 52 or a pharmaceutically acceptable salt thereof, wherein the group: 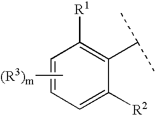
in formula I is selected from 2,6-dichlorophenyl, 2-chlorophenyl, 2-chloro-4-fluorophenyl, 2-chloro-6-fluorophenyl, 2-chloro-6-methylphenyl, 2-fluoro-6-methylphenyl, 2-chloro-4-methoxyphenyl, 2-ethyl-4-fluorophenyl and 2,6-dimethylphenyl.

58. A compound selected from:
N-(2,6-dichlorobenzoyl)-O-[2-(pyridin-2-ylamino)ethyl]-L-tyrosine;
N-(2,6-dichlorobenzoyl)-O-[3-(pyridin-2-ylamino)propyl]-L-tyrosine;
N-(2,6-dichlorobenzoyl)-O-[4-(pyridin-2-ylamino)butyl]-L-tyrosine;
N-(2,6-dichlorobenzoyl)-O-(5,6,7,8-tetrahydro-1,8-naphthyridin-2-ylmethyl)-L-tyrosine;
N-(2,6-dichlorobenzoyl)-O-[2-(5,6,7,8-tetrahydro-1,8-naphthyridin-2-yl)ethyl]-L-tyrosine;
N-(2,6-dichlorobenzoyl)-O-{[6-(methylamino)pyridin-2-yl]methyl}-L-tyrosine;
N-(2,6-dichlorobenzoyl)-O-{2-[6-(methylamino)pyridin-2-yl]ethyl}-L-tyrosine;
N-(2,6-dichlorobenzoyl)-O-{2-[6-(methylamino)pyridin-2-yl]butyl}-L-tyrosine;
N-(2,6-dichlorobenzoyl)-O-{3-[6-(methylamino)pyridin-2-yl]propyl}-L-tyrosine;
N-(2,6-dichlorobenzoyl)-4-[3-(pyridin-2-ylamino)prop-1-yn-1-yl]-L-phenylalanine;
N-(2,6-dichlorobenzoyl)-4-[3-(pyridin-2-ylamino)propyl]-L-phenylalanine;
N-(2,6-dichlorobenzoyl)-4-[5-(pyridin-2-ylamino)pent-1-yn-1-yl]-L-phenylalanine;
O-[2-(1H-benzimidazol-2-ylamino)ethyl]-N-(2,6-dichlorobenzoyl)-L-tyrosine;
N-(2-chlorobenzoyl)-O-[3-(pyridin-2-ylamino)propyl]-L-tyrosine;
N-(2,4-dichlorobenzoyl)-O-[3-(pyridin-2-ylamino)propyl]-L-tyrosine;
N-(2-chloro-5-methylbenzoyl)-O-[3-(pyridin-2-ylamino)propyl]-L-tyrosine;
N-(2-chloro-6-fluorobenzoyl)-O-[3-(pyridin-2-ylamino)propyl]-L-tyrosine;
N-(2-chloro-6-fluoro-3-methylbenzoyl)-O-[3-(pyridin-2-ylamino)propyl]-L-tyrosine;
N-(6-chloro-2-fluoro-3-methylbenzoyl)-O-[3-(pyridin-2-ylamino)propyl]-L-tyrosine;
N-(3-chloro-2-methylbenzoyl)-O-[3-(pyridin-2-ylamino)propyl]-L-tyrosine;
N-(2-chloro-3,6-difluorobenzoyl)-O-[3-(pyridin-2-ylamino)propyl]-L-tyrosine;
N-(2-methylbenzoyl)-O-[3-(pyridin-2-ylamino)propyl]-L-tyrosine;
N-(mesitylcarbonyl)-O-[3-(pyridin-2-ylamino)propyl]-L-tyrosine;
N-(2,6-dichlorobenzoyl)-4-[4-(pyridin-2-ylamino)but-1-yn-1-yl]-L-phenylalanine; and
N-(2,6-dichlorobenzoyl)-3-{5-[2-(5,6,7,8-tetrahydro-1,8-naphthyridin-2-yl)ethoxy]pyridin-2-yl}-L-alanine;
or a pharmaceutically acceptable salt thereof.

59. A compound selected from:
N-(2,6-dichloro-3-methylbenzoyl)-O-[3-(pyridin-2-ylamino)propyl]-L-tyrosine
N-(2,6-dimethylbenzoyl)-O-[3-(pyridin-2-ylamino)propyl]-L-tyrosine;
N-[(5-chloro-1,3-benzodioxol-4-yl)carbonyl]-O-[3-(pyridin-2-ylamino)propyl]-L-tyrosine;
N-(2-chloro-4-fluorobenzoyl)-O-[3-(pyridin-2-ylamino)propyl]-L-tyrosine;
N-(2-bromo-6-chlorobenzoyl)-O-[3-(pyridin-2-ylamino)propyl]-L-tyrosine;
N-(2,6-dichlorobenzoyl)-3-[3-(pyridin-2-ylamino)propoxy]-L-phenylalanine;
N-(2,6-dichlorobenzoyl)-O-[2-(1-methyl-1,2,3,4-tetrahydropyrido[2,3-b]pyrazin-6-yl)ethyl]-L-tyrosine;
N-(2,6-dichlorobenzoyl)-4-{3-[6-(methylamino)pyridin-2-yl]propyl}-L-phenylalanine;
N-(2,6-dichlorobenzoyl)-4-{3-[6-(methylamino)pyridin-2-yl]propyl}-L-phenylalanine;
N-(2-chlorobenzoyl)-4-{3-[6-(methylamino)pyridin-2-yl]propyl}-L-phenylalanine;
N-(2-chloro-6-fluorobenzoyl)-4-{3-[6-(methylamino)pyridin-2-yl]propyl}-L-phenylalanine;
N-(2-chloro-4-fluorobenzoyl)-4-{3-[6-(methylamino)pyridin-2-yl]propyl}-L-phenylalanine;
N-(2-chloro-6-fluoro-3-methylbenzoyl)-4-{3-[6-(methylamino)pyridin-2-yl]propyl}-L-phenylalanine;
N-(2,6-dichlorobenzoyl)-3-fluoro-O-{2-[6-(methylamino)pyridin-2-yl]ethyl}-L-tyrosine;
N-(2,6-dichlorobenzoyl)-3-methyl-O-{2-[6-(methylamino)pyridin-2-yl]ethyl}-L-tyrosine;
N-(2,6-dichlorobenzoyl)-3,5-dimethyl-O-{2-[6-(methylamino)pyridin-2-yl]ethyl}-L-tyrosine;
N-(2,6-dichlorobenzoyl)-O-{3-[(5-methylpyridin-2-yl)amino]propyl}-L-tyrosine;
N-(2,6-dichlorobenzoyl)-O-{3-[(4-methylpyridin-2-yl)amino]propyl}-L-tyrosine;
N-(2,6-dichlorobenzoyl)-O-{3-[(6-methylpyridin-2-yl)amino]propyl}-L-tyrosine;
N-(2,6-dichlorobenzoyl)-O-{3-[(3-methylpyridin-2-yl)amino]propyl}-L-tyrosine;
N-(2,6-dichlorobenzoyl)-O-{3-[(4,6-dimethylpyridin-2-yl)amino]propyl}-L-tyrosine;
N-(2,6-dichlorobenzoyl)-O-{3-[(4-ethylpyridin-2-yl)amino]propyl}-L-tyrosine;
N-(2,6-dichlorobenzoyl)-4-[4-(pyridin-2-ylamino)butyl]-L-phenylalanine;
N-(2,6-dichlorobenzoyl)-O-[2-(5,6,7,8-tetrahydro-1,8-naphthyridin-2-yl)propyl]-L-tyrosine;
N-(2,6-dichlorobenzoyl)-O-[2-methyl-2-(5,6,7,8-tetrahydro-1,8-naphthyridin-2-yl)propyl]-L-tyrosine;
N-(2-chloro-6-fluorobenzoyl)-O-[2-(3,4-dihydro-2H-pyrido[3,2-b][1,4]oxazin-6-yl)ethyl]-L-tyrosine;
N-(2-chlorobenzoyl)-O-[2-(3,4-dihydro-2H-pyrido[3,2-b][1,4]oxazin-6-yl)ethyl]-L-tyrosine;
N-(2-chloro-4-fluorobenzoyl)-O-[2-(3,4-dihydro-2H-pyrido[3,2-b][1,4]oxazin-6-yl)ethyl]-L-tyrosine;
N-(2-chloro-6-fluoro-3-methylbenzoyl)-O-[2-(3,4-dihydro-2H-pyrido[3,2-b][1,4]oxazin-6-yl)ethyl]-L-tyrosine;
N-(2-chloro-6-fluorobenzoyl)-O-[2-(3,4-dihydro-2H-pyrido[3,2-b][1,4]oxazin-6-yl)ethyl]-L-tyrosine; and
N-(2,6-dichlorobenzoyl)-3-(5-{3-[6-(methylamino)pyridin-2-yl]propyl}-2-thienyl)-L-alanine;
or a pharmaceutically acceptable salt thereof.

60. A compound selected from:
N-[(2,6-dichlorophenyl)carbonyl]-4-({[2-(pyridin-2-ylamino)ethyl]amino}methyl)-L-phenylalanine;
N-[(2,6-dichlorophenyl)carbonyl]-O-{3-[(5-methoxypyridin-2-yl)amino]propyl}-L-tyrosine;
N-[(2,6-dichlorophenyl)carbonyl]-O-{3-[(4-methoxypyridin-2-yl)amino]propyl}-L-tyrosine;
N-[(2,6-dichlorophenyl)carbonyl]-O-{2-[5-methoxy-6-(methylamino)pyridin-2-yl]ethyl}-L-tyrosine;
N-[(2,6-dichlorophenyl)carbonyl]-O-[2-(4-methyl-5,6,7,8-tetrahydro-1,8-naphthyridin-2-yl)ethyl]-L-tyrosine;
N-[(2,6-dichlorophenyl)carbonyl]-O-[2-(7-methyl-5,6,7,8-tetrahydro-1,8-naphthyridin-2-yl)ethyl]-L-tyrosine;
N-[(2-chlorophenyl)carbonyl]-4-[3-(3,4-dihydro-2H-pyrido[3,2-b][1,4]oxazin-6-yl)propyl]-L-phenylalanine;
N-[(2-chlorophenyl)carbonyl]-4-[3-(3,4-dihydro-2H-pyrido[3,2-b][1,4]oxazin-6-yl)propyl]-L-phenylalanine;
N-[(2-chloro-6-fluorophenyl)carbonyl]-4-[3-(3,4-dihydro-2H-pyrido[3,2-b][1,4]oxazin-6-yl)propyl]-L-phenylalanine;
N-[(2-chloro-4-fluorophenyl)carbonyl]-4-[3-(3,4-dihydro-2H-pyrido[3,2-b][1,4]oxazin-6-yl)propyl]-L-phenylalanine;
N-[(2,6-dichlorophenyl)carbonyl]-4-[3-(3,4-dihydro-2H-pyrido[3,2-b][1,4]oxazin-6-yl)propyl]-L-phenylalanine;
N-[(2,6-dichlorophenyl)carbonyl]-4-({[6-(methylamino)pyridin-2-yl]methoxy}methyl)-L-phenylalanine;
N-[(2,6-dichlorophenyl)carbonyl]-4-{[2-(methylamino)quinolin-7-yl]methyl}-L-phenylalanine;
N-[(2,4-dichlorophenyl)carbonyl]-O-{2-[6-(methylamino)pyridin-2-yl]ethyl}-L-tyrosine;
N-[(2-chloro-3,6-difluorophenyl)carbonyl]-O-{2-[6-(methylamino)pyridin-2-yl]ethyl}-L-tyrosine;
N-[(2-chloro-5-methylphenyl)carbonyl]-O-{2-[6-(methylamino)pyridin-2-yl]ethyl}-L-tyrosine;
O-{2-[6-(methylamino)pyridin-2-yl]ethyl}-N-[(2,4,6-trimethylphenyl)carbonyl]-L-tyrosine;
N-[(2-chloro-6-methylphenyl)carbonyl]-O-{2-[6-(methylamino)pyridin-2-yl]ethyl}-L-tyrosine;
N-[(2-chloro-4-fluorophenyl)carbonyl]-O-{2-[6-(methylamino)pyridin-2-yl]ethyl}-L-tyrosine;
N-[(2-chloro-6-fluorophenyl)carbonyl]-O-{2-[6-(methylamino)pyridin-2-yl]ethyl}-L-tyrosine;
N-[(2,6-dimethylphenyl)carbonyl]-O-{2-[6-(methylamino)pyridin-2-yl]ethyl}-L-tyrosine;
N-[(6-chloro-2-fluoro-3-methylphenyl)carbonyl]-O-{2-[6-(methylamino)pyridin-2-yl]ethyl}-L-tyrosine;
O-{2-[6-(methylamino)pyridin-2-yl]ethyl}-N-[(2-methylphenyl)carbonyl]-L-tyrosine;
N-[(2-chlorophenyl)carbonyl]-O-{2-[6-(methylamino)pyridin-2-yl]ethyl}-L-tyrosine;
N-[(5-chloro-1,3-benzodioxol-4-yl)carbonyl]-O-{2-[6-(methylamino)pyridin-2-yl]ethyl}-L-tyrosine;
N-[(2-fluoro-6-methylphenyl)carbonyl]-O-{2-[6-(methylamino)pyridin-2-yl]ethyl}-L-tyrosine;
N-[(2,3-dichlorophenyl)carbonyl]-O-{2-[6-(methylamino)pyridin-2-yl]ethyl}-L-tyrosine;
N-{[5-fluoro-2-(trifluoromethyl)phenyl]carbonyl}-O-{2-[6-(methylamino)pyridin-2-yl]ethyl}-L-tyrosine;
O-{2-[6-(methylamino)pyridin-2-yl]ethyl}-N-{[2-(trifluoromethyl)phenyl]carbonyl}-L-tyrosine;
N-[(2,5-dimethylphenyl)carbonyl]-O-{2-[6-(methylamino)pyridin-2-yl]ethyl}-L-tyrosine;
N-[(2-ethylphenyl)carbonyl]-O-{2-[6-(methylamino)pyridin-2-yl]ethyl}-L-tyrosine;
N-[(3-chloro-2-methylphenyl)carbonyl]-O-{2-[6-(methylamino)pyridin-2-yl]ethyl}-L-tyrosine;
N-{[4-(acetylamino)-2-methylphenyl]carbonyl}-O-{2-[6-(methylamino)pyridin-2-yl]ethyl}-L-tyrosine;
N-{[2-fluoro-6-(trifluoromethyl)phenyl]carbonyl}-O-{2-[6-(methylamino)pyridin-2-yl]ethyl}-L-tyrosine;
N-[(2-chloro-6-fluoro-3-methylphenyl)carbonyl]-O-{2-[6-(methylamino)pyridin-2-yl]ethyl}-L-tyrosine;
N-[(4-chloro-2-methylphenyl)carbonyl]-O-{2-[6-(methylamino)pyridin-2-yl]ethyl}-L-tyrosine;
N-{[2-(acetylamino)-6-chlorophenyl]carbonyl}-O-{2-[6-(methylamino)pyridin-2-yl]ethyl}-L-tyrosine;
N-[(2,3-dimethylphenyl)carbonyl]-O-{2-[6-(methylamino)pyridin-2-yl]ethyl}-L-tyrosine;
N-[(2,4-dimethylphenyl)carbonyl]-O-{2-[6-(methylamino)pyridin-2-yl]ethyl}-L-tyrosine;
N-[(4-methoxy-2-methylphenyl)carbonyl]-O-{2-[6-(methylamino)pyridin-2-yl]ethyl}-L-tyrosine;
N-{[4-fluoro-2-(trifluoromethyl)phenyl]carbonyl}-O-{2-[6-(methylamino)pyridin-2-yl]ethyl}-L-tyrosine;
N-[(5-fluoro-2-methylphenyl)carbonyl]-O-{2-[6-(methylamino)pyridin-2-yl]ethyl}-L-tyrosine;
N-[(3,6-dichloro-2-methoxyphenyl)carbonyl]-O-{2-[6-(methylamino)pyridin-2-yl]ethyl}-L-tyrosine;
N-{[2-chloro-5-(trifluoromethyl)phenyl]carbonyl}-O-{2-[6-(methylamino)pyridin-2-yl]ethyl}-L-tyrosine;
N-[(3-fluoro-2-methylphenyl)carbonyl]-O-{2-[6-(methylamino)pyridin-2-yl]ethyl}-L-tyrosine;
N-[(2,4-dichloro-5-fluorophenyl)carbonyl]-O-{2-[6-(methylamino)pyridin-2-yl]ethyl}-L-tyrosine;
O-{2-[6-(methylamino)pyridin-2-yl]ethyl}-N-[(2,4,5-trimethylphenyl)carbonyl]-L-tyrosine;
N-[(2-chloro-4,5-difluorophenyl)carbonyl]-O-{2-[6-(methylamino)pyridin-2-yl]ethyl}-L-tyrosine;
N-[(2-chloro-3,4-dimethoxyphenyl)carbonyl]-O-{2-[6-(methylamino)pyridin-2-yl]ethyl}-L-tyrosine;
N-{[2-chloro-4-(methylsulfonyl)phenyl]carbonyl}-O-{2-[6-(methylamino)pyridin-2-yl]ethyl}-L-tyrosine;
N-[(3-hydroxy-2-methylphenyl)carbonyl]-O-{2-[6-(methylamino)pyridin-2-yl]ethyl}-L-tyrosine;
N-{[2-chloro-3-(trifluoromethyl)phenyl]carbonyl}-O-{2-[6-(methylamino)pyridin-2-yl]ethyl}-L-tyrosine;
N-{[3-chloro-2-fluoro-6-(trifluoromethyl)phenyl]carbonyl}-O-{2-[6-(methylamino)pyridin-2-yl]ethyl}-L-tyrosine;
N-{[5-chloro-2-(trifluoromethyl)phenyl]carbonyl}-O-{2-[6-(methylamino)pyridin-2-yl]ethyl}-L-tyrosine;
N-[(3-methoxy-2-methylphenyl)carbonyl]-O-{2-[6-(methylamino)pyridin-2-yl]ethyl}-L-tyrosine;
N-[(4-fluoro-2-methylphenyl)carbonyl]-O-{2-[6-(methylamino)pyridin-2-yl]ethyl}-L-tyrosine;
N-[(2-chloro-4-fluoro-5-sulfamoylphenyl)carbonyl]-O-{2-[6-(methylamino)pyridin-2-yl]ethyl}-L-tyrosine;
N-[(2-chloro-3-methylphenyl)carbonyl]-O-{2-[6-(methylamino)pyridin-2-yl]ethyl}-L-tyrosine;
N-[(2-chloro-4-methoxyphenyl)carbonyl]-O-{2-[6-(methylamino)pyridin-2-yl]ethyl}-L-tyrosine;
N-[(2-chloro-4-pyrrolidin-1-ylphenyl)carbonyl]-O-{2-[6-(methylamino)pyridin-2-yl]ethyl}-L-tyrosine;
N-[(2-chloro-4-morpholin-4-ylphenyl)carbonyl]-O-{2-[6-(methylamino)pyridin-2-yl]ethyl}-L-tyrosine;
N-{[2-chloro-4-(1H-pyrazol-1-yl)phenyl]carbonyl}-O-{2-[6-(methylamino)pyridin-2-yl]ethyl}-L-tyrosine;
N-[(2-chloro-5-methoxyphenyl)carbonyl]-O-{2-[6-(methylamino)pyridin-2-yl]ethyl}-L-tyrosine;
N-[(2-chloro-4,5-dimethoxyphenyl)carbonyl]-O-{2-[6-(methylamino)pyridin-2-yl]ethyl}-L-tyrosine;
O-{2-[6-(methylamino)pyridin-2-yl]ethyl}-N-{[2-methyl-3-(trifluoromethyl)phenyl]carbonyl}-L-tyrosine;
N-[(5-chloro-2-methylphenyl)carbonyl]-O-{2-[6-(methylamino)pyridin-2-yl]ethyl}-L-tyrosine;
O-{2-[6-(methylamino)pyridin-2-yl]ethyl}-N-{[2-methyl-5-(trifluoromethyl)phenyl]carbonyl}-L-tyrosine;
N-{[3-fluoro-2-(trifluoromethyl)phenyl]carbonyl}-O-{2-[6-(methylamino)pyridin-2-yl]ethyl}-L-tyrosine;
N-{[4-methoxy-2-(trifluoromethyl)phenyl]carbonyl}-O-{2-[6-(methylamino)pyridin-2-yl]ethyl}-L-tyrosine;
N-[(2-chloro-3-fluorophenyl)carbonyl]-O-{2-[6-(methylamino)pyridin-2-yl]ethyl}-L-tyrosine;
N-[(2-chloro-5-fluorophenyl)carbonyl]-O-{2-[6-(methylamino)pyridin-2-yl]ethyl}-L-tyrosine;
N-[(2-ethyl-4-fluorophenyl)carbonyl]-O-{2-[6-(methylamino)pyridin-2-yl]ethyl}-L-tyrosine;
N-[(4,5-difluoro-2-methylphenyl)carbonyl]-O-{2-[6-(methylamino)pyridin-2-yl]ethyl}-L-tyrosine;
N-[(2-ethyl-5-fluorophenyl)carbonyl]-O-{2-[6-(methylamino)pyridin-2-yl]ethyl}-L-tyrosine;
N-[(2-cyclopropyl-4-fluorophenyl)carbonyl]-O-{2-[6-(methylamino)pyridin-2-yl]ethyl}-L-tyrosine;
N-[(2,4-dichlorophenyl)carbonyl]-O-[2-(5,6,7,8-tetrahydro-1,8-naphthyridin-2-yl)ethyl]-L-tyrosine;
N-[(2-chloro-3,6-difluorophenyl)carbonyl]-O-[2-(5,6,7,8-tetrahydro-1,8-naphthyridin-2-yl)ethyl]-L-tyrosine;
N-[(2-chloro-5-methylphenyl)carbonyl]-O-[2-(5,6,7,8-tetrahydro-1,8-naphthyridin-2-yl)ethyl]-L-tyrosine;
O-[2-(5,6,7,8-tetrahydro-1,8-naphthyridin-2-yl)ethyl]-N-[(2,4,6-trimethylphenyl)carbonyl]-L-tyrosine;
N-[(2-chloro-6-methylphenyl)carbonyl]-O-[2-(5,6,7,8-tetrahydro-1,8-naphthyridin-2-yl)ethyl]-L-tyrosine;
N-[(2-chloro-4-fluorophenyl)carbonyl]-O-[2-(5,6,7,8-tetrahydro-1,8-naphthyridin-2-yl)ethyl]-L-tyrosine;
N-[(2-chloro-6-fluorophenyl)carbonyl]-O-[2-(5,6,7,8-tetrahydro-1,8-naphthyridin-2-yl)ethyl]-L-tyrosine;
N-[(2,6-dimethylphenyl)carbonyl]-O-[2-(5,6,7,8-tetrahydro-1,8-naphthyridin-2-yl)ethyl]-L-tyrosine;
N-[(6-chloro-2-fluoro-3-methylphenyl)carbonyl]-O-[2-(5,6,7,8-tetrahydro-1,8-naphthyridin-2-yl)ethyl]-L-tyrosine;
N-[(2-chlorophenyl)carbonyl]-O-[2-(5,6,7,8-tetrahydro-1,8-naphthyridin-2-yl)ethyl]-L-tyrosine;
N-[(5-chloro-1,3-benzodioxol-4-yl)carbonyl]-O-[2-(5,6,7,8-tetrahydro-1,8-naphthyridin-2-yl)ethyl]-L-tyrosine;
N-[(2-methylphenyl)carbonyl]-O-[2-(5,6,7,8-tetrahydro-1,8-naphthyridin-2-yl)ethyl]-L-tyrosine;
N-[(2-fluoro-6-methylphenyl)carbonyl]-O-[2-(5,6,7,8-tetrahydro-1,8-naphthyridin-2-yl)ethyl]-L-tyrosine;
N-[(2-chlorophenyl)carbonyl]-3-(5-{3-[6-(methylamino)pyridin-2-yl]propyl}pyridin-2-yl)-L-alanine;
N-[(2,6-dichlorophenyl)carbonyl]-3-(5-{3-[6-(methylamino)pyridin-2-yl]propyl}pyridin-2-yl)-L-alanine;
N-[(2-chlorophenyl)carbonyl]-4-{4-[6-(methylamino)pyridin-2-yl]butyl}-L-phenylalanine;
N-[(2,6-dichlorophenyl)carbonyl]-4-{4-[6-(methylamino)pyridin-2-yl]butyl}-L-phenylalanine;
N-[(2,6-dichlorophenyl)carbonyl]-O-[2-(5-methyl-5,6,7,8-tetrahydro-1,8-naphthyridin-2-yl)ethyl]-L-tyrosine;
N-[(2-chlorophenyl)carbonyl]-4-[3-(5,6,7,8-tetrahydro-1,8-naphthyridin-2-yl)propyl]-L-phenylalanine;
N-[(2,6-dichlorophenyl)carbonyl]-O-{2-[6-(dimethylamino)pyridin-2-yl]ethyl}-L-tyrosine;
O-{2-[6-(cyclopentylamino)pyridin-2-yl]ethyl}-N-[(2,6-dichlorophenyl)carbonyl]-L-tyrosine;
O-{2-[6-(cyclopropylamino)pyridin-2-yl]ethyl}-N-[(2,6-dichlorophenyl)carbonyl]-L-tyrosine;
O-[2-(6-aminopyridin-2-yl)ethyl]-N-[(2,6-dichlorophenyl)carbonyl]-L-tyrosine;
O-(2-{6-[(cyclopropylmethyl)amino]pyridin-2-yl}ethyl)-N-[(2,6-dichlorophenyl)carbonyl]-L-tyrosine;
N-[(2,6-dichlorophenyl)carbonyl]-O-(2-{6-[(2-methoxyethyl)amino]pyridin-2-yl}ethyl)-L-tyrosine; and
N-[(2,6-dichlorophenyl)carbonyl]-O-[2-(6-{[2-(dimethylamino)ethyl]amino}pyridin-2-yl)ethyl]-L-tyrosine;
N-[(2-bromo-4-fluorophenyl)carbonyl]-O-{2-[6-(methylamino)pyridin-2-yl]ethyl}-L-tyrosine;
N-[(2-bromo-4,5-difluorophenyl)carbonyl]-O-{2-[6-(methylamino)pyridin-2-yl]ethyl}-L-tyrosine;
N-[(2-bromo-5-fluorophenyl)carbonyl]-O-{2-[6-(methylamino)pyridin-2-yl]ethyl}-L-tyrosine;
N-[(2,6-dichlorophenyl)carbonyl]-3-(6-{2-[6-(methylamino)pyridin-2-yl]ethoxy}pyridin-3-yl)-L-alanine;
N-[(2-chloro-4-fluorophenyl)carbonyl]-3-(6-{2-[6-(methylamino)pyridin-2-yl]ethoxy}pyridin-3-yl)-L-alanine;
N-[(2-chloro-4-methoxyphenyl)carbonyl]-3-(6-{2-[6-(methylamino)pyridin-2-yl]ethoxy}pyridin-3-yl)-L-alanine;
N-[(2-ethyl-4-fluorophenyl)carbonyl]-3-(6-{2-[6-(methylamino)pyridin-2-yl]ethoxy}pyridin-3-yl)-L-alanine;
N-[(2,6-dichlorophenyl)carbonyl]-3-(6-{3-[6-(methylamino)pyridin-2-yl]propyl}pyridin-3-yl)-L-alanine;
N-[(2,6-dichlorophenyl)carbonyl]-3-(4-{3-[6-(methylamino)pyridin-2-yl]propyl}thiophen-2-yl)-L-alanine;
N-[(2,6-dichlorophenyl)carbonyl]-3-(4-{2-[6-(methylamino)pyridin-2-yl]ethoxy}thiophen-2-yl)-L-alanine;
N-[(2,6-dimethylphenyl)carbonyl]-3-(5-{3-[6-(methylamino)pyridin-2-yl]propyl}thiophen-2-yl)-L-alanine;
N-[(2-chloro-6-fluorophenyl)carbonyl]-3-(5-{3-[6-(methylamino)pyridin-2-yl]propyl}thiophen-2-yl)-L-alanine;
O-[2-(3,4-dihydro-2H-pyrido[3,2-b][1,4]oxazin-6-yl)ethyl]-N-[(2,6-dimethylphenyl)carbonyl]-L-tyro sine;
N-[(2-chloro-6-methylphenyl)carbonyl]-O-[2-(3,4-dihydro-2H-pyrido[3,2-b][1,4]oxazin-6-yl)ethyl]-L-tyrosine;
O-[2-(3,4-dihydro-2H-pyrido[3,2-b][1,4]oxazin-6-yl)ethyl]-N-[(2-ethyl-4-fluorophenyl)carbonyl]-L-tyro sine;
O-[2-(3,4-dihydro-2H-pyrido[3,2-b][1,4]oxazin-6-yl)ethyl]-N-[(2,4,6-trimethylphenyl)carbonyl]-L-tyrosine ;
N-[(2-chloro-4-methoxyphenyl)carbonyl]-O-[2-(3,4-dihydro-2H-pyrido[3,2-b][1,4]oxazin-6-yl)ethyl]-L-tyrosine;
N-[(2-chloro-5-fluorophenyl)carbonyl]-O-[2-(3,4-dihydro-2H-pyrido[3,2-b][1,4]oxazin-6-yl)ethyl]-L-tyrosine;
O-[2-(3,4-dihydro-2H-pyrido[3,2-b][1,4]oxazin-6-yl)ethyl]-N-[(2-ethyl-5-fluorophenyl)carbonyl]-L-tyro sine;
N-[(2-chloro-6-fluorophenyl)carbonyl]-3-(5-{3-[6-(methylamino)pyridin-2-yl]propyl}pyridin-2-yl)-L-alanine;
N-[(2-chloro-4-fluorophenyl)carbonyl]-3-(5-{3-[6-(methylamino)pyridin-2-yl]propyl}pyridin-2-yl)-L-alanine;
N-[(2-ethyl-4-fluorophenyl)carbonyl]-3-(5-{3-[6-(methylamino)pyridin-2-yl]propyl}pyridin-2-yl)-L-alanine;
N-[(2,6-dichlorophenyl)carbonyl]-3-{6-[2-(5,6,7,8-tetrahydro-1,8-naphthyridin-2-yl)ethoxy]pyridin-3-yl}-L-alanine;
N-[(2-chlorophenyl)carbonyl]-3-{6-[2-(5,6,7,8-tetrahydro-1,8-naphthyridin-2-yl)ethoxy]pyridin-3-yl}-L-alanine;
N-[(2-chloro-4-fluorophenyl)carbonyl]-3-{6-[2-(5,6,7,8-tetrahydro-1,8-naphthyridin-2-yl)ethoxy]pyridin-3-yl}-L-alanine;
N-[(2-chloro-6-fluorophenyl)carbonyl]-3-{6-[2-(5,6,7,8-tetrahydro-1,8-naphthyridin-2-yl)ethoxy]pyridin-3-yl}-L-alanine;
N-[(2-chlorophenyl)carbonyl]-3-{5-[2-(5,6,7,8-tetrahydro-1,8-naphthyridin-2-yl)ethoxy]pyridin-2-yl}-L-alanine;
N-[(2-chloro-4-fluorophenyl)carbonyl]-3-{5-[2-(5,6,7,8-tetrahydro-1,8-naphthyridin-2-yl)ethoxy]pyridin-2-yl}-L-alanine;
N-[(2-chloro-6-fluorophenyl)carbonyl]-3-{5-[2-(5,6,7,8-tetrahydro-1,8-naphthyridin-2-yl)ethoxy]pyridin-2-yl}-L-alanine;
N-[(2-chloro-4-methoxyphenyl)carbonyl]-3-{5-[2-(5,6,7,8-tetrahydro-1,8-naphthyridin-2-yl)ethoxy]pyridin-2-yl}-L-alanine;
N-[(2-fluoro-4-methylphenyl)carbonyl]-3-{5-[2-(5,6,7,8-tetrahydro-1,8-naphthyridin-2-yl)ethoxy]pyridin-2-yl}-L-alanine;
N-[(2-ethyl-4-fluorophenyl)carbonyl]-3-{5-[2-(5,6,7,8-tetrahydro-1,8-naphthyridin-2-yl)ethoxy]pyridin-2-yl}-L-alanine;
N-[(2-chlorophenyl)carbonyl]-3-(5-{2-[6-(methylamino)pyridin-2-yl]ethoxy}pyridin-2-yl)-L-alanine;
N-[(2-chloro-4-fluorophenyl)carbonyl]-3-(5-{2-[6-(methylamino)pyridin-2-yl]ethoxy}pyridin-2-yl)-L-alanine;
N-[(2-chloro-6-fluorophenyl)carbonyl]-3-(5-{2-[6-(methylamino)pyridin-2-yl]ethoxy}pyridin-2-yl)-L-alanine;
N-[(2-chloro-4-methoxyphenyl)carbonyl]-3-(5-{2-[6-(methylamino)pyridin-2-yl]ethoxy}pyridin-2-yl)-L-alanine;
N-[(2-ethyl-4-fluorophenyl)carbonyl]-3-(5-{2-[6-(methylamino)pyridin-2-yl]ethoxy}pyridin-2-yl)-L-alanine;
N-[(2,6-dichlorophenyl)carbonyl]-3-(5-{2-[6-(methylamino)pyridin-2-yl]ethoxy}pyridin-2-yl)-L-alanine;
N-[(2,6-dichlorophenyl)carbonyl]-3-{5-[3-(5,6,7,8-tetrahydro-1,8-naphthyridin-2-yl)propyl]pyridin-2-yl}-L-alanine;
N-[(2-chlorophenyl)carbonyl]-3-{5-[3-(5,6,7,8-tetrahydro-1,8-naphthyridin-2-yl)propyl]thiophen-2-yl}-L-alanine;
N-[(2-chloro-4-fluorophenyl)carbonyl]-3-{5-[3-(5,6,7,8-tetrahydro-1,8-naphthyridin-2-yl)propyl]thiophen-2-yl}-L-alanine;
N-[(2-chloro-6-fluorophenyl)carbonyl]-3-{5-[3-(5,6,7,8-tetrahydro-1,8-naphthyridin-2-yl)propyl]thiophen-2-yl}-L-alanine;
N-[(2,6-dichlorophenyl)carbonyl]-3-{5-[3-(5,6,7,8-tetrahydro-1,8-naphthyridin-2-yl)propyl]thiophen-2-yl}-L-alanine;
N-[(2-chloro-6-fluorophenyl)carbonyl]-4-[3-(5,6,7,8-tetrahydro-1,8-naphthyridin-2-yl)propyl]-L-phenylalanine;
N-[2,6-dichlorophenyl)carbonyl]-4-[3-(5,6,7,8-tetrahydro-1,8-naphthyridin-2-yl)propyl]-L-phenylalanine;
N-[(2-chloro-4-fluorophenyl)carbonyl]-4-[3-(5,6,7,8-tetrahydro-1,8-naphthyridin-2-yl)propyl]-L-phenylalanine;
N-[(2-chlorophenyl)carbonyl]-3-{5-[3-(5,6,7,8-tetrahydro-1,8-naphthyridin-2-yl)propyl]pyridin-2-yl}-L-alanine;
N-[(2-fluoro-6-methylophenyl)carbonyl]-3-{5-[3-(5,6,7,8-tetrahydro-1,8-naphthyridin-2-yl)propyl]pyridin-2-yl}-L-alanine; and
N-[(2-chloro-4-fluorophenyl)carbonyl]-3-{5-[3-(5,6,7,8-tetrahydro-1,8-naphthyridin-2-yl)propyl]pyridin-2-yl}-L-alanine;
or a pharmaceutically acceptable salt thereof.

61. A compound selected from:
N-[(2-chloro-6-fluorophenyl)carbonyl]-3-{5-[3-(5,6,7,8-tetrahydro-1,8-naphthyridin-2-yl)propyl]pyridin-2-yl}-L-alanine;
N-[(2,6-dichlorophenyl)carbonyl]-3-{6-[3-(5,6,7,8-tetrahydro-1,8-naphthyridin-2-yl)propyl]pyridin-3-yl}-L-alanine;
N-[(2-chlorophenyl)carbonyl]-3-{6-[3-(5,6,7,8-tetrahydro-1,8-naphthyridin-2-yl)propyl]pyridin-3-yl}-L-alanine;
N-[(2-chloro-4-fluorophenyl)carbonyl]-3-{6-[3-(5,6,7,8-tetrahydro-1,8-naphthyridin-2-yl)propyl]pyridin-3-yl}-L-alanine; and
N-[(2-chloro-6-fluorophenyl)carbonyl]-3-{6-[3-(5,6,7,8-tetrahydro-1,8-naphthyridin-2-yl)propyl]pyridin-3-yl}-L-alanine;
or a pharmaceutically acceptable salt thereof.

62. A pharmaceutical composition which comprises a compound of the formula I, or a pharmaceutically acceptable thereof, as defined in claim 1 in association with a pharmaceutically-acceptable diluent or carrier.

63. A method of inhibiting a5b1 function comprising administering to an animal or human in need of said inhibiting a therapeutically effective amount of a compound of formula I, or a pharmaceutically acceptable salt, as claimed in claim 1.

64. A method of treatment of pathological angiogenesis in a human or animal in need of such treatment comprising administering to said human or animal a therapeutically effective amount of a compound of formula I or a pharmaceutically acceptable salt thereof, as claimed in claim 1.

65. A method of treatment of a cancer comprising administering to a human or animal in need of such treatment a therapeutically effective amount of a compound of formula I, or a pharmaceutically acceptable salt thereof, as claimed in claim 1.

66. A method of treatment of a cancer comprising administering to a human or animal in need of such treatment a therapeutically effective amount of:
(a) a compound of formula I, or a pharmaceutically acceptable salt thereof, as claimed in claim 1; and
(b) an additional chemotherapeutic agent.

67. A process for the preparation of a compound according to claim 1 or a pharmaceutically acceptable salt thereof which comprises:
Process (a) for the preparation of those compounds of formula I wherein Z is N(R²⁶), O or S, by reacting a compound of the formula II: 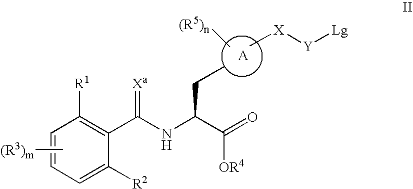
wherein R¹, R², R³, R⁴, R⁵, X^{a}, A, Z, Y, n and m are as defined in claim 1, except any functional group is protected if necessary, and
Lg is a displaceable group,
with a compound of the formula III:
R⁶-ZH III
wherein R⁶ and Z are as defined in claim 1, except any functional group is protected if necessary; or
Process (b) for the preparation of those compounds of the formula I wherein X is O, the coupling of a compound of the formula IV: 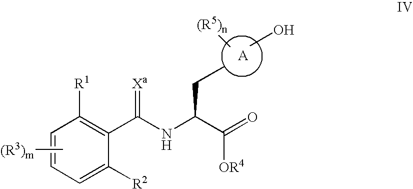
wherein R¹, R², R³, R⁴, R⁵, X^{a}, A, n and m are as defined in claim 1, except any functional group is protected if necessary,
with a compound of the formula V:
R⁶-Z-Y—OH V
wherein R⁶, Y and Z are as defined in claim 1, except any functional group is protected if necessary; or
Process (c) for the preparation of those compounds of formula I wherein X is O, N(R²⁶) or S by reacting a compound of the formula VI: 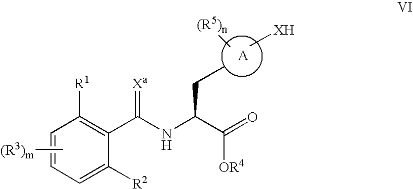
wherein R¹, R², R³, R⁴, R⁵, X^{a}, A, n and m are as defined in claim 1, except any functional group is protected if necessary,
with a compound of the formula VII:
R⁶-Z-Y-Lg¹ VII
wherein R⁶, Y and Z are as defined in claim 1, except any functional group is protected if necessary, and
Lg¹ is a displaceable group; or
Process (d) for the preparation of those compounds of the formula I wherein Z is —C═C—, —C≡C— or the group —Y-Z is alkylene the reaction of a compound of the formula VIII: 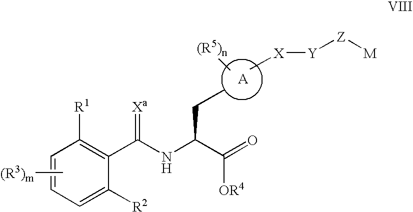
wherein R¹, R², R³, R⁴, R⁵, A, X^{a}, X, Y, Z, n and m are as defined in claim 1, except any functional group is protected if necessary,
and M is a suitable displaceable group,
with a compound of the formula R⁶Lg²,
wherein R⁶ is as defined in claim 1, except any functional group is protected if necessary, and
Lg² is a displaceable group; or
Process (f) for compounds of formula I where X^{a} is oxygen, the coupling of a compound of the formula XI: 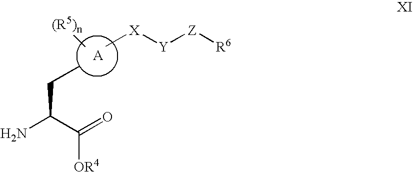
wherein R⁴, R⁵, R⁶, A, X, Y, Z and n are as defined in claim 1, except any functional group is protected if necessary,
with a compound of the formula XII, or a reactive derivative thereof: 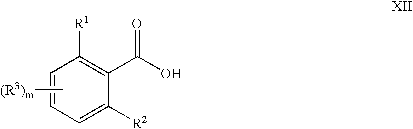
wherein R¹, R², R³ and m are as defined in claim 1, except any functional group is protected if necessary; or
Process (h) for the preparation of those compounds of the formula I wherein X is N(R²⁶), O or S, or where X is a direct bond and Y is a heterocyclic group which is linked to the group A in formula I via a ring nitrogen atom, the coupling of a compound of the formula XV: 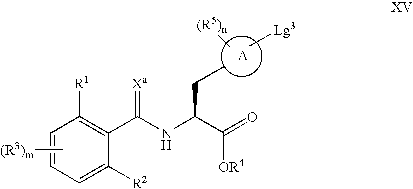
wherein R¹, R², R³, R⁴, R⁵, X^{a}, A, n and m are as defined in claim 1, except any functional group is protected if necessary, and
Lg³ is a suitable displaceable group,
with a compound of the formula XVI:
R⁶-Z-Y—X—H XVI
wherein R⁶, X, Y and Z are as defined in claim 1, except any functional group is protected if necessary; or
Process (i) for the preparation of those compounds of the formula I wherein X is —C═C—, —C≡C— or the group —X—Y is alkylene the coupling of a compound of the formula XV: 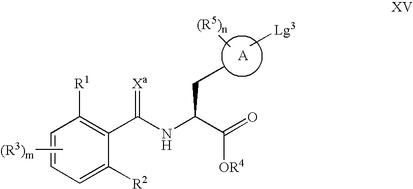
wherein R¹, R², R³, R⁴, R⁵, X^{a}, A, Lg³, n and m are as defined in claim 1, except any functional group is protected if necessary,
with a compound of the formula XVII:
R⁶-Z-Y—X-M XVII
wherein R⁶, Y and Z are as defined in claim 1, except any functional group is protected if necessary,
and M is a suitable displaceable group; or
Process (j) for the preparation of those compounds of the formula I wherein Z is N(R²⁶), the coupling of a compound of the formula XVIII: 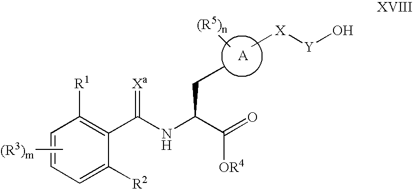
wherein R¹, R², R³, R⁴, R⁵, A, X^{a}, X, Y, n and m are as defined in claim 1, except any functional group is protected if necessary,
with a compound of the formula XIX:
R⁶—N(R²⁶)H XIX
wherein R⁶ and R²⁶ are as defined in claim 1, except any functional group is protected if necessary; or
Process (k) for the preparation of those compounds of formula I wherein Z is N(R²⁶), O or S, by reacting a compound of the formula XX: 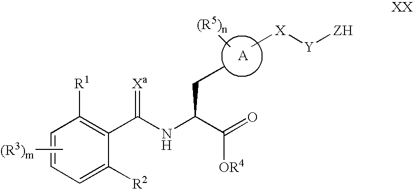
wherein R¹, R², R³, R⁴, R⁵, A, X^{a}, X, Y, Z, n and m are as defined in claim 1, except any functional group is protected if necessary,
with a compound of the formula XXI:
R⁶-Lg XXI
wherein R⁶ is as defined in claim 1, except any functional group is protected if necessary, and
Lg is a displaceable group; or
Process (l) for the preparation of those compounds of the formula I wherein the group —X—Y-Z- contains an alkylene chain of at least 3 carbon atoms in length, the hydrogenation of an unsaturated product of Process (d) or (i) described herein; or
Process (m) for the preparation of those compounds of the formula I where X^{a} is a sulfur, by reacting a compound of formula XXII: 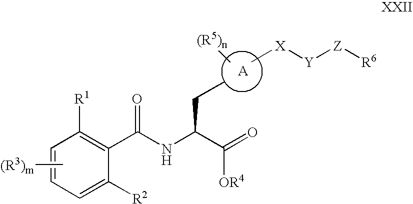
wherein R¹, R², R³, R⁴, R⁵, R⁶, A, X, Y, Z, n and m are as defined in claim 1, except any functional group is protected if necessary,
with a thiation reagent; or
Process (n) for the preparation of those compounds of the formula I wherein —X—Y-Z- contains a (1-6C)alkoxy or substituted (1-6C)alkoxy group or a (1-6C)alkylamino or substituted (1-6C)alkylamino group, the alkylation, conveniently in the presence of a suitable base as defined hereinbefore, of the corresponding alcohol or amine in which X, Y or Z contains a hydroxy group or a primary or secondary amino group as appropriate; or a reductive amination in which X, Y or Z contains a primary or secondary amino group as appropriate; or
Process (o) when ring A is thiophenyl and R⁶ is 5,6,7,8-tetrahydro-1,8-naphthyridin-2-yl; the reduction of a compound of the formula XXIV: 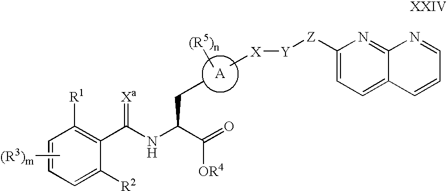
wherein A, R¹, R², R³, R⁴, R⁵, X, Y, Z, n and m are as defined in claim 1, except any functional group is protected if necessary;
and thereafter, if necessary (in any order):
(i) converting a compound of the formula I into another compound of the formula I;
(ii) removing any protecting groups; and
(iii) forming a pharmaceutically acceptable salt of the compound of formula I.
